(19) United States
(12) Patent Application Publication
(10) Pub. No.: US 2014/0243286 A1
(43) Pub. Date: Aug. 28, 2014

(54) BROMODOMAIN LIGANDS CAPABLE OF DIMERIZING IN AN AQUEOUS SOLUTION, AND METHODS OF USING SAME

(71) Applicant: Coferon, Inc., Stony Brook, NY (US)

(72) Inventors: Lee Daniel Arnold, Mount Sinai, NY (US); Kenneth W. Foreman, Syosset, NY (US); Douglas S. Werner, Babylon, NY (US)

(73) Assignee: Coferon, Inc., Stony Brook, NY (US)

(21) Appl. No.: 14/193,522

(22) Filed: Feb. 28, 2014

Related U.S. Application Data
(63) Continuation of application No. PCT/US12/52943, filed on Aug. 29, 2012.
(60) Provisional application No. 61/587,857, filed on Jan. 18, 2012.
(60) Provisional application No. 61/528,474, filed on Aug. 29, 2011.

(51) Int. Cl. A61K 31/695; A61K 31/4706; A61K 31/166; A61K 31/69; A61K 31/551

(52) U.S. Cl. CPC A61K 31/695; A61K 31/69; A61K 31/551; A61K 31/166; A61K 31/4706
USPC 514/63; 540/541; 514/64; 546/13; 540/566; 514/220; 564/179; 514/621; 546/162; 514/313; 546/159; 546/14; 540/543

(57) ABSTRACT

Described herein are monomers capable of forming a biologically useful multimer when in contact with one, two, three or more other monomers in an aqueous media. In one aspect, such monomers may be capable of binding to another monomer in an aqueous media (e.g. in vivo) to form a multimer (e.g. a dimer). Contemplated monomers may include a ligand moiety, a linker element, and a connector element that joins the ligand moiety and the linker element. In an aqueous media, such contemplated monomers may join together via each linker element and may thus be capable of modulating one or more biomolecules substantially simultaneously, e.g., modulate two or more binding domains on a protein or on different proteins.

### CROSS REFERENCE TO RELATED APPLICATIONS

This application is a continuation of International Application No. PCT/US12/52943, filed Aug. 29, 2012, which claims priority to U.S. Provisional Application No. 61/528,474, filed Aug. 29, 2011, and U.S. Provisional Application No. 61/587,857, filed Jan. 18, 2012, each of which is hereby incorporated by reference in its entirety.

### BACKGROUND

Current drug design and drug therapies have not addressed the urgent need for therapies that interact with extended areas or multiple domains of biomolecules such as proteins. For example, few therapies exist that can modulate protein-protein interactions, e.g., by interacting, simultaneously, with two domains on a single protein or with both a domain on one protein and a domain on another protein. There is also an urgent need for such therapies that modulate fusion proteins, such as those that occur in cancer.

Signaling pathways are used by cells to generate biological responses to external or internal stimuli. A few thousand gene products control both ontogeny/development of higher organisms and sophisticated behavior by their many different cell types. These gene products can work in different combinations to achieve their goals and often do so through protein-protein interactions. Such proteins possess modular protein domains that recognize, bind, and/or modify certain motifs. For example, some proteins include tandem or repeating domains.

The BET family of bromodomain containing proteins bind to acetylated lysine residues in histones and other proteins to influence transcription, etc. Proteins in the BET family are typically characterized by having tandem bromodomains. Exemplary protein targets having tandem bromodomains include BRD4, a member of the BET family. BRD4 is also a proto-oncogene that can be mutated via chromosomal translocation in a rare form of squamous cell carcinoma. Further, proteins having tandem bromodomains such as BRD4 may be suitable as a drug target for other indications such as acute myeloid leukemia. Bromodomains are typically small domains having e.g., about 110 amino acids. Bromodomain modulators may be useful for various diseases or conditions, including those relating to systemic or tissue inflammation, inflammatory response to infection, malignant cell activation and proliferation, lipid metabolism, cell differentiation, and prevention and treatment of viral infections.

Current drug design and drug therapy approaches typically focus on modulating one protein domain with limited selectivity and do not address the urgent need to find drugs that are capable of modulating such tandem domains substantially simultaneously in order to further improve on specificity and potency. Although antibodies and other biological therapeutic agents may have sufficient specificity to distinguish among closely related protein surfaces, factors such as their high molecular weight prevent oral administration and cellular uptake of the antibodies. Conversely, orally active pharmaceuticals are generally too small to effectively disrupt protein-protein surface interactions, which can be much larger than the orally active pharmaceuticals. Further, previous attempts to link multiple, e.g., two, pharmacophores that each interact with, e.g., different protein domains, have focused on large covalently linked compounds assembled in organic solvents. These assemblies typically have a molecular weight too large for oral administration or effective cellular and tissue permeation.

### SUMMARY

Described herein, for example, are monomers capable of forming a biologically useful multimer when in contact with one, two, three or more other monomers in an aqueous media. In one aspect, such monomers may be capable of binding to another monomer in an aqueous media (e.g. in vivo) to form a multimer, (e.g., a dimer). Contemplated monomers may include a ligand moiety (e.g., a pharmacophore for the target biomolecule), a linker element, and a connector element that joins the ligand moiety and the linker element. In an aqueous media, such contemplated monomers may join together via each linker element and may thus be capable of modulating one or more biomolecules substantially simultaneously, e.g., modulate two or more binding domains on a protein or on different proteins.

In one aspect, a first monomer capable of forming a biologically useful multimer capable of modulating a protein having a first bromodomain when in contact with a second monomer in an aqueous media is provided. Such a first monomer may be represented by the formula:
X¹—Y¹—Z¹ (Formula I) and pharmaceutically acceptable salts, stereoisomers, metabolites, and hydrates thereof, wherein
   X¹ is a first ligand moiety capable of modulating the first bromodomain on said protein;
   Y¹ is absent or is a connector moiety covalently bound to X¹ and Z¹;
   Z¹ is a first linker capable of binding to the second monomer; and the second monomer is represented by the formula:
X²—Y²—Z² (Formula II) and pharmaceutically acceptable salts, stereoisomers, metabolites, and hydrates thereof, wherein
   X² is a second ligand moiety capable of modulating a second domain on said protein;
   Y² is absent or is a connector moiety covalently bound to X² and Z²; and
   Z² is a second linker capable of binding to the first monomer through Z¹.

In another aspect, a therapeutic multimer compound formed from the multimerization in an aqueous media of a first monomer and a second monomer is provided. Such a first monomer may be represented by:

X¹—Y¹—Z¹ (Formula I)

and the second monomer represented by

X²—Y²—Z² (Formula II),

wherein
X¹ is a first ligand moiety capable of modulating a first bromodomain;
Y¹ is absent or is a connector moiety covalently bound to X¹ and Z¹;
Z¹ is a first linker capable of binding to Z² to form the multimer;
X² is a second ligand moiety capable of modulating a second protein domain;
Y² is absent or is a connector moiety covalently bound to X² and Z²; and
Z² is a boronic acid or oxaborale moiety capable of binding with the Z¹ moiety of Formula I to form the multimer; and
   pharmaceutically acceptable salts, stereoisomers, metabolites and hydrates thereof

In yet another aspect, a first monomer is provided, wherein the first monomer is represented by the formula X³—Y³—Z³ (Formula III) and pharmaceutically acceptable salts, stereoisomers, metabolites and hydrates thereof, wherein

X³ is a first ligand moiety capable of modulating a bromodomain;

Y³ is absent or is a connector moiety covalently bound to X³ and Z³; and

Z³ is a linker capable of forming a therapeutic multimer with another monomer or other monomers of Formula III, wherein Z³ is the same for the first and second monomer.

In still another aspect, a method of treating a disease associated with a protein having tandem bromodomains in a patient in need thereof is provided. Such a disclosed method can include administering to said patient a first monomer represented by:

X¹—Y¹—Z¹ (Formula I) and pharmaceutically acceptable salts, stereoisomers, metabolites and hydrates thereof, wherein X¹ is a first ligand moiety capable of modulating a first bromodomain; and administering to said patient a second monomer represented by: X²—Y²—Z² (Formula II), wherein X² is a second ligand moiety capable of modulating a second bromodomain, wherein upon administration, said first monomer and said second monomer forms a multimer in vivo that binds to the first and the second bromodomain.

### BRIEF DESCRIPTION OF THE DRAWINGS

FIG. 1 shows a screenshot of a protein X-ray crystal structure in which the structures of I-BET762 and an isoxazole pharmacophore are overlaid, according to an embodiment.

FIG. 2 shows a non-limiting set of pharmacophores (i.e., ligands) with preferred attachment points for connecting the pharmacophores to connecting moieties indicated by arrows, according to an embodiment.

### DETAILED DESCRIPTION

Described herein, for example, are monomers capable of forming a biologically useful multimer when in contact with one, two, three or more other monomers in an aqueous media. In one aspect, such monomers may be capable of binding to another monomer in an aqueous media (e.g., in vivo) to form a multimer, (e.g., a dimer). Contemplated monomers may include a ligand moiety (e.g., a pharmacophore moiety), a linker element, and a connector element that joins the ligand moiety and the linker element. In an aqueous media, such contemplated monomers may join together via each linker element and may thus be capable of modulating one or more biomolecules substantially simultaneously, e.g., modulate two or more binding domains on a protein or on different proteins.

For example, contemplated monomers may be separate or separatable in a solid or in an aqueous media under one set of conditions, and when placed in an aqueous media having one or more biomolecules (e.g., under a different set of conditions) can 1) form a multimer with another monomer through the linker on each monomer; and either: 2a) bind to the biomolecule in two or more locations (e.g., protein domains) through each ligand moiety of the respective monomer or 2b) bind to two or more biomolecules through each ligand moiety of the respective monomer. In an exemplary embodiment, disclosed monomers may interact with another appropriate monomer (i.e., a monomeric pair) in an aqueous media (e.g., in vivo) to form a multimer (e.g., a dimer) that can bind to two separate target biomolecule domains (e.g., protein domains). In one embodiment, the two separate target domains can be tandem domains on the same target, for example, tandem BET bromodomains.

The ligand moiety of a contemplated monomer, in some cases, may be a pharmacophore or a ligand moiety that is, e.g., capable of binding to and/or modulating a biomolecule, such as, for example, a protein, e.g, a specific protein domain, a component of a biological cell, such as a ribosome (composed of proteins and nucleic acids) or an enzyme active site (e.g., a protease, such as tryptase). In some embodiments, the linker element comprises a functional group capable of forming a chemical bond with another linker element. In some embodiments, the linker moiety may also serve as a signaling entity or “reporter,” and in some instances the assembly of two or more linkers can produce a fluorescent entity or fluorophore with properties distinct from the individual linker moiety. In another aspect, a plurality of monomers, each comprising a linker element, may react to form a multimer connected by the linker elements. In some embodiments, the multimer may be formed in vivo. In some instances, the multimer may have enhanced properties relative to the monomers that form the multimer. For example, in certain embodiments, the multimer may bind to a target with greater affinity than any of the monomers that form the multimer. Also described are methods of making the compositions and methods of administering the compositions.

In some embodiments, the first ligand moiety may be capable of binding to a bromodomain. For example, in some embodiments, X¹, X², X³ and X⁴ of Formula I, II, III or IV may each be capable of binding to a bromodomain in a protein selected from the group consisting of BRD2 D2, BRD3 D2, BRD4 D2, BRD-t D2, yBdf1 D2, yBdf2 D2, KIAA2026, yBdf1 D1, yBdf2 D1, TAF1L D1, TAF1 D1, TAF1L D2, TAF1 D2, ZMYND8, ZMYND11, ASH1L, PBRM D3, PBRM D1, PBRM D2, PBRM D4, PBRM D5, SMARCA2, SMARCA4 ySnf2, ySth, PBRM D6, yRsc1 D2, yRsc2 D2, yRsc1 D1, yRsc2 D1, yRsc4 D1, BRWD1 D1, BRWD3 D1, PHIP D1, MLL, MLL4, BRWD2, ATAD2, ATAD2B, BRD1, BRPF1, BRPF3, BRD7, BRD9, BAZ1B, BRWD1 D2, PHIP D2, BRWD3, CREBBP, EP300 BRD8 D1, BRD8 D2, yRsc4 D2, ySpt7, BAZ1A, BAZ2A, BAZ2B, SP140, SP140L, TRIM28, TRIM24, TRIM33, TRIM66, BPTF, GCN5L2, PCAF, yGcn5, BRD2 D1, BRD3 D1, BRD4 D1, BRD-t D1 and CECR2. Reference to protein and domain names used herein are derived from Zhang Q, Chakravarty S, Ghersi D, Zeng L, Plotnikov A N, et al. (2010) Biochemical Profiling of Histone Binding Selectivity of the Yeast Bromodomain Family. PLoS ONE 5(1): e8903. doi:10.1371/journal.pone.0008903. In some embodiments, multimers contemplated herein may be capable of binding to a tandem bromodomain. For example, in some cases, a multimer may be capable of binding to a tandem bromodomain in a protein selected from the group consisting of BRD2, BRD3, BRD4 and BRD-t.

In some embodiments, the second ligand moiety may also be capable of binding to a bromodomain. In certain embodiments, the second ligand moiety may be capable of binding to epigenetically associated domains. Non-limiting examples of epigenetically associated domains include HATs (acetyl transferases), bromodomains (acetyl readers), HDACs (deacetylases), Methyltransferases (PRMTs, KMTs, DNMTs), Methyl readers (Chromo, Tudor, MBT, PHD, PWWP, WD40), Methyl erasers (K-specific demethylases, JmJC, MethylCytosine hydroxylase), kinases, phosphate readers (14-3-3, WD40, BRCT), phosphatases, Citruline writers (Protein arginine deiminase), SANT/MYB domain, BAH, E3 ligases, SUMO ligases, RING domain, HECT domain, and lysine biotinases.

In yet other instances, the second ligand moiety may be capable of binding to domains such as methyl transferases, ATPases, ubiquinases, histone acetyl transferases, methyl readers (PWWP, WD40), protein adaptors (extraterminal domains, MYND), and DNA binders (zinc fingers, BBOX).

In some embodiments, a plurality of monomers may assemble to form a multimer. The multimer may be used for a variety of purposes. For example, in some instances, the multimer may be used to perturb a biological system. As described in more detail below, in some embodiments, the multimer may bind to or modulate a target biomolecule, such as a protein, nucleic acid, or polysaccharide. In certain embodiments, a contemplated multimer may be used as a pharmaceutical.

Advantageously, in some embodiments, a multimer may form in vivo upon administration of suitable monomers to a subject. Also advantageously, the multimer may be capable of interacting with a relatively large target site as compared to the individual monomers that form the multimer. For example, a target may comprise, in some embodiments, two protein domains separated by a distance such that a multimer, but not a monomer, may be capable of binding to both domains essentially simultaneously. In some embodiments, contemplated multimers may bind to a target with greater affinity as compared to a monomer binding affinity alone.

In some embodiments, a contemplated multimer may advantageously exhibit enhanced properties relative to the monomers that form the multimer. As discussed above, a multimer may have improved binding properties as compared to the monomers alone. In some embodiments, a multimer may have improved signaling properties. For example, in some cases, the fluorescent properties of a multimer may be different as compared to a monomer. In some embodiments, the fluorescent brightness of a multimer at a particular wavelength may be significantly different (e.g., greater) than the fluorescent brightness at the same wavelength of the monomers that form the multimer. Advantageously, in some embodiments, a difference in signaling properties between the multimer and the monomers that form the multimer may be used to detect formation of the multimer. In some embodiments, detection of the formation of the multimer may be used to screen monomers, as discussed in more detail below. Also as discussed in more detail below, in some embodiments, the multimers may be used for imaging or as diagnostic agents.

It should be understood that a multimer, as used herein, may be a homomultimer (i.e., a multimer formed from two or more essentially identical monomers) or may be a heteromultimer (i.e., a multimer formed from two or more substantially different monomers). In some embodiments, a contemplated multimer may comprise 2 to about 10 monomers, for example, a multimer may be a dimer, a trimer, a tetramer, or a pentamer.

In some embodiments, a monomer may comprise a ligand moiety, a linker element, and a connector element that associates the ligand moiety with the linker element. In some embodiments, the linker element of a first monomer may combine with the linker element of a second monomer. In some cases, the linker element may comprise a functional group that can react with a functional group of another linker element to form a bond linking the monomers. In some embodiments, the linker element of a first monomer may be substantially the same as the linker element of a second monomer. In some embodiments, the linker element of a first monomer may be substantially different than the linker element of a second monomer.

In some cases, the ligand moiety may be a pharmacophore. In some embodiments, the ligand moiety (e.g., a pharmacophore) may bind to a target molecule with a dissociation constant of less than 1 mM, in some embodiments less than 500 microM, in some embodiments less than 300 microM, in some embodiments less than 100 microM, in some embodiments less than 10 microM, in some embodiments less than 1 microM, in some embodiments less than 100 nM, in some embodiments less than 10 nM, and in some embodiments less than 1 nM.

In some embodiments, the IC₅₀ of the first monomer against a first target biomolecule and the IC₅₀ of the second monomer against a second target biomolecule may be greater than the apparent IC₅₀ of a combination of the monomers against the first target biomolecule and the second target biomolecule. The combination of monomers may be any suitable ratio. For example, the ratio of the first monomer to the second monomer may be between 10:1 to 1:10, in some embodiments between 5:1 and 1:5, and in some embodiments between 2:1 and 1:2. In some cases, the ratio of the first monomer to the second monomer may be essentially 1:1. In some instances, the ratio of the smaller of the IC₅₀ of the first monomer and the second monomer to the apparent IC₅₀ of the multimer may be at least 3.0. In other instances, the ratio of the smaller IC₅₀ of the first monomer or the second monomer to the apparent IC₅₀ of the multimer may be at least 10.0. In some embodiments, the ratio of the smaller IC₅₀ of the first monomer or the second monomer to the apparent IC₅₀ of the multimer may be at least 30.0.

For example, for disclosed monomers forming a heteromultimer, the apparent IC₅₀ resulting from an essentially equimolar combination of monomers against the first target biomolecule and the second target biomolecule may be, in some embodiments, at least about 3 to 10 fold lower, at least about 10 to 30 fold lower, at least about 30 fold lower, or at least about 40 to 50 fold lower than the lowest of the IC₅₀ of the second monomer against the second target biomolecule or the IC₅₀ of the first monomer against the first target biomolecule.

It will be appreciated that for monomers forming homodimers (or homo-oligomeric or homomultimeric, as described below), in aqueous solution, there may be an equilibrium between the monomeric and dimeric (or oligomeric) states with higher concentrations favoring greater extent of oligomer (e.g., dimer) formation. As the binding of monomers to the target biomolecule increases their proximity and effectively increases their local concentration on the target, the rate and extent of dimerization (oligomerization) is promoted when geometries are favorable. As a result, the occupancy of the target by favorable monomers may be nearly completely in the homodimeric (or oligomeric) state. In this manner the target, for example, may serve as a template for the dimerization (or oligomerization) of the monomers, significantly enhancing the extent and rate of dimerization.

While the affinity of the multimer for its target biomolecule(s) often cannot be measured directly due to the dynamic reversible equilibrium with its monomers in an aqueous or biological milieu, it may be possible to extract an apparent multimer-target dissociation constant from a series of experimental determinations. Exploring the effects of a matrix of monomer concentrations, monomer ratios, along with changes in concentration(s) in the target biomolecule(s), coupled with determinations of multimer-monomer dissociation constants, and in some cases additional binding competition, kinetic and biophysical methods, one can extract an estimate of the affinity of the multimeric assembly for its target(s). Through such approaches, one can demonstrate that in some embodiments, the affinity of the multimer for the target biomolecule(s) are less than 1 μM, in some embodiments, less than 1 nM, in some embodiments, less than 1 pM, in some embodiments, less than 1 fM, and in some embodiments, less than 1 aM, and in some embodiments, less than 1 zM.

Affinities of heterodimerizing monomers for the target biomolecule can be assessed through the testing of the respective monomers in appropriate assays for the target activity or biology because they do not typically self-associate. In contrast, the testing of homodimerizing monomers may not, in some embodiments, afford an affinity for the monomeric or dimeric state, but rather the observed effect (e.g. IC₅₀) is a result of the monomer-dimer dynamics and equilibrium, with the apparent binding affinity (or IC₅₀) being, e.g., a weighted measure of the monomer and dimeric inhibitory effects upon the target.

In some cases, the pH of the aqueous fluid in which the multimer forms may be between pH 1 and 9, in some embodiments, between pH 1 and 3, in some embodiments, between pH 3 and 5, in some embodiments, between pH 5 and 7, and in some embodiments, between pH 7 and 9. In some embodiments, the multimer may be stable in an aqueous solution having a pH between pH 1 and 9, in some embodiments between pH 1 and 3, in some embodiments between pH 3 and 5, in some embodiments between pH 5 and 7, and in some embodiments between pH 7 and 9. In some embodiments, the aqueous solution may have a physiologically acceptable pH.

In some embodiments, the ligand moiety may be capable of binding to a target and at least partially disrupting a biomolecule-biomolecule interaction (e.g., a protein-protein interaction). In some embodiments, the ligand moiety may be capable of binding to a target and at least partially disrupting a protein-nucleic acid interaction. In some cases, the ligand moiety may be capable of binding to a target and at least partially disrupting a protein-lipid interaction. In some cases, the ligand moiety may be capable of binding to a target and at least partially disrupting a protein-polysaccharide interaction. In some embodiments, the ligand moiety may be capable of at least partially stabilizing a biomolecule-biomolecule interaction. In certain embodiments, the ligand moiety may be capable of at least partially inhibiting a conformational change in a biomolecule target.

In some instances, the linker element may be capable of generating a signal. For example, in some embodiments, the linker element may be capable of fluorescing. In some cases, the linker element may have greater fluorescence when the monomer to which it is attached is part of a multimer as compared to when the monomer to which it is attached is not part of a multimer. In some embodiments, upon multimer formation, the fluorescent brightness of a linker element may increase by at least 2-fold, in some embodiments, by at least 5-fold, in some embodiments, by at least 10-fold, in some embodiments, by at least 50-fold, in some embodiments, by at least 100-fold, in some embodiments, by at least 1000-fold, and in some embodiments, by at least 10000-fold. In some embodiments, a linker element in a multimer may have a peak fluorescence that is red-shifted relative to the peak fluorescence of the linker element in a monomer. In other embodiments, a linker element may have a peak fluorescence that is blue-shifted relative to the peak fluorescence of a linker element in a monomer.

### Monomers

In certain embodiments, a first monomer may be capable of forming a biologically useful multimer capable of modulating a protein having a bromodomain when in contact with a second monomer in an aqueous media. For example, a first monomer may be represented by the formula:

X¹—Y¹—Z¹ (Formula I) and pharmaceutically acceptable salts, stereoisomers, metabolites, and hydrates thereof, wherein
X¹ is a first ligand moiety capable of binding to or modulating a bromodomain on said protein;
Y¹ is absent or is a connector moiety covalently bound to X¹ and Z¹;
Z¹ is a first linker capable of binding to the second monomer; and a second monomer may be represented by the formula:

X²—Y²—Z² (Formula II) and pharmaceutically acceptable salts, stereoisomers, metabolites, and hydrates thereof, wherein
X² is a second ligand moiety capable of binding to a second domain on said protein;
Y² is absent or is a connector moiety covalently bound to X² and Z²; and
Z² is a second linker capable of binding to the first monomer through Z¹.

For example, when a first and second monomer capable of forming a multimer (e.g., dimer) when in contact in an aqueous solution each has a different linker, e.g., Z¹ and Z² are different, the monomers may be referred to as ‘hetero’ monomers.

In one embodiment, X¹ and X² are the same. In another embodiment, X¹ and X² are different.

In certain embodiments, the protein is independently selected from the group consisting of BRD2, BRD3, BRD4 and BRD-t. In another example, the second domain is a second bromodomain. For example, the second domain is a bromodomain within 50 Å of the first bromodomain.

In another embodiment, a monomer may be represented by the formula:

X³—Y³—Z³ (Formula III) and pharmaceutically acceptable salts, stereoisomers, metabolites, and hydrates thereof, wherein
X³ is a ligand moiety capable of binding to a bromodomain;
Y³ is absent or is a connector moiety covalently bound to X³ and Z³;
Z³ is a linker capable of binding to one or more Z³ moieties from other X³—Y³—Z³ monomers to form a biologically useful multimer.

In a certain embodiment, a first monomer is capable of forming a biologically useful multimer when in contact with a second monomer in an aqueous media, wherein the first monomer is represented by the formula:

X¹—Y¹—Z¹ (Formula I) and pharmaceutically acceptable salts, stereoisomers, metabolites, and hydrates thereof, wherein
X¹ is a first ligand moiety capable of binding to a bromodomain;
Y¹ is absent or is a connector moiety covalently bound to X¹ and Z¹;
Z¹ is a first linker capable of binding to the second monomer (e.g., in-vivo); and the second monomer is represented by the formula:

X⁴—Y⁴—Z⁴ (Formula IV) and pharmaceutically acceptable salts, stereoisomers, metabolites, and hydrates thereof, wherein
X⁴ is a second ligand moiety capable of binding to a protein domain, wherein the protein domain is e.g., within about 10, 20, 30, 40, 50, 60, 70, 80 or more A, e.g. within about 50 Å of the bromodomain (e.g the protein domain may be another bromodomain, or may be a different type of domain such as the NUT portion of a BRD-NUT fusion protein);
Y⁴ is absent or is a connector moiety covalently bound to X⁴ and Z⁴; and
Z⁴ is a second linker capable of binding to the first monomer through Z¹.

In another certain embodiment, a first monomer may be capable of forming a biologically useful multimer when in contact with one, two, three or more monomers (e.g. a first silyl monomer and a second silyl monomer). For example, a first and second monomer may be represented by the formula:

X³—Y³—Z³ (Formula III) and pharmaceutically acceptable salts, stereoisomers, metabolites and hydrates thereof, wherein

X³ is a first ligand moiety capable of binding to and modulating a first target biomolecule (e.g., bromodomain);

Y³ is absent or is a connector moiety covalently bound to X³ and Z³;

Z³ is linker capable of forming a therapeutic multimer (e.g., dimer) with another monomer or other monomers of Formula III, wherein Z³ is the same for the first and second monomer, as noted below. For example, when a first and second monomer capable of forming a multimer (e.g., dimer) when in contact in an aqueous solution and each monomer have the same linker, e.g., Z³, the monomers may be referred to as ‘homo’ monomers.

### A) Linkers

The linker moieties Z¹, Z², Z³ and Z⁴ of Formulas I, II, III and IV may, in some embodiments, be the same or different.

In a certain embodiment, the first monomer is represented by the formula

X¹—Y¹—Z¹, wherein Z¹ is a first linker that, for example, may form a dimer with a second monomer, e.g., X²—Y²—Z² or X⁴—Y⁴—Z⁴, wherein, for example, Z¹ may be a diol and Z² or Z⁴ may independently be a boronic acid or oxaborole moiety. In one embodiment, Z¹ is a first linker selected from the group consisting of

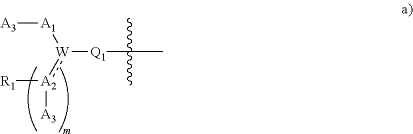

wherein

A₁ is (a) absent; or (b) selected from the group consisting of acyl, substituted or unsubstituted aliphatic, or substituted or unsubstituted heteroaliphatic;

A₂, independently for each occurrence, is (a) absent; or (b) selected from the group consisting of —N—, acyl, substituted or unsubstituted aliphatic, or substituted or unsubstituted heteroaliphatic, provided that at least one of A₁ and A₂ is present; or

A₁ and A₂, together with the atoms to which they are attached, form a substituted or unsubstituted 4-8 membered cycloalkyl or heterocyclic ring;

A₃ is selected from the group consisting of —NHR′, —SH, or —OH;

W is CR′ or N;

R′ is selected from the group consisting of hydrogen, halogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH;

m is 1-6;

[symbol] represents a single or double bond; and

R₁ is (a) absent; or (b) selected from the group consisting of hydrogen, halogen, substituted or unsubstituted aliphatic, or substituted or unsubstituted heteroaliphatic, substituted or unsubstituted phenyl or naphthyl, substituted or unsubstituted heteroaryl, —NH₂, —NO₂, —SH, or —OH;

Q₁ is (a) absent; or (b) selected from the group consisting of substituted or unsubstituted aliphatic or substituted or unsubstituted heteroaliphatic; or

R₁ and Q₁ together with the atoms to which they are attached form a substituted or unsubstituted 4-8 membered cycloalkyl or heterocyclic ring;

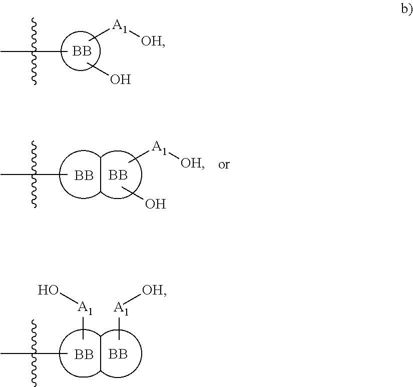

wherein

BB, independently for each occurrence, is a 4-8 membered cycloalkyl, heterocyclic, phenyl, naphthyl, or heteroaryl moiety, wherein the cycloalkyl, heterocyclic, phenyl, naphthyl, or heteroaryl moiety is optionally substituted with one or more groups represented by R₂, wherein the two substituents comprising —OH have a 1,2 or 1,3 configuration;

each R₂ is independently selected from hydrogen, halogen, oxo, sulfonate, —NO₂, —CN, —OH, —NH₂, —SH, —COOH, —CONHR′, substituted or unsubstituted aliphatic, substituted or unsubstituted heteroaliphatic, or two R₂ together with the atoms to which they are attached form a fused substituted or unsubstituted 4-6 membered cycloalkyl or heterocyclic bicyclic ring system;

A₁, independently for each occurrence, is (a) absent; or (b) selected from the group consisting of acyl, substituted or unsubstituted aliphatic, or substituted or unsubstituted heteroaliphatic;

R′ is selected from the group consisting of hydrogen, halogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH;

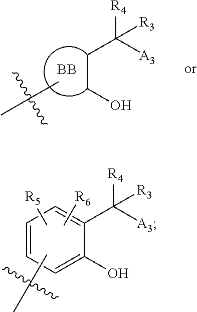

wherein

BB is a substituted or unsubstituted 5- or 6-membered cycloalkyl, heterocyclic, phenyl, naphthyl, or heteroaryl moiety;

A₃, independently for each occurrence, is selected from the group consisting of —NHR′ or —OH;

R₃ and R₄ are independently selected from the group consisting of H, C₁₋₄alkyl, phenyl, or R₃ and R₄ taken together from a 3-6 membered ring;

R₅ and R₆ are independently selected from the group consisting of H, C₁₋₄alkyl optionally substituted by hydroxyl, amino, halogen, or thio; C₁₋₄alkoxy; halogen; —OH; —CN; —COOH; —CONHR′; or R₅ and R₆ taken together form phenyl or a 4-6 membered heterocycle; and

R′ is selected from the group consisting of hydrogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH;

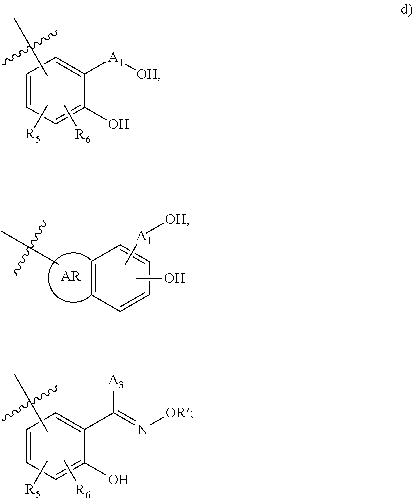

wherein

A₁ is (a) absent; or (b) selected from the group consisting of acyl, substituted or unsubstituted aliphatic, or substituted or unsubstituted heteroaliphatic;

A₃, independently for each occurrence, is selected from the group consisting of —NHR′ or —OH;

AR is a fused phenyl or 4-7 membered aromatic or partially aromatic heterocyclic ring, wherein AR is optionally substituted by oxo, C₁₋₄alkyl optionally substituted by hydroxyl, amino, halo, or thio; C₁₋₄alkoxy; —S—C₁₋₄alkyl; halogen; —OH; —CN; —COOH; —CONHR′; wherein the two substituents comprising —OH are ortho to each other;

R₅ and R₆ are independently selected from the group consisting of H, C₁₋₄alkyl optionally substituted by hydroxyl, amino, halo, or thio; C₁₋₄alkoxy; halogen; —OH; —CN; —COOH; CONHR′; and

R′ is selected from the group consisting of hydrogen, halogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH;

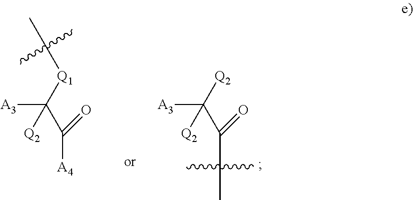

wherein

Q₁ is selected from the group consisting of C₁₋₄alkyl, alkylene, or a bond; C₁₋₆cycloalkyl; a 5-6 membered heterocyclic ring; or phenyl;

Q₂, independently for each occurrence, is selected from the group consisting of H, C₁₋₄alkyl, alkylene, or a bond; C₁₋₆cycloalkyl; a 5-6 membered heterocyclic ring; substituted or unsubstituted aliphatic; substituted or unsubstituted heteroaliphatic; substituted or unsubstituted phenyl or naphthyl; or substituted or unsubstituted heteroaryl;

A₃, independently for each occurrence, is selected from the group consisting of —NH₂ or —OH;

A₄, independently for each occurrence, is selected from the group consisting of —NH—NH₂; —NHOH, —NH—OR″, or —OH;

R″ is selected from the group consisting of H or C₁₋₄alkyl; and

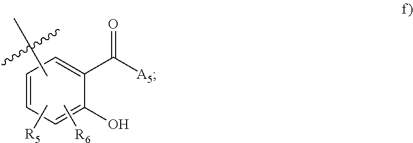

wherein

A₅ is selected from the group consisting of —OH, —NH₂, —SH, —NHR′″;

R′″ is selected from —NH₂; —OH; phenoxy; heteroaryloxy; and C₁₋₄alkoxy;

R₅ and R₆ are independently selected from the group consisting of H, C₁₋₄alkyl optionally substituted by hydroxyl, amino, halo, or thio; C₁₋₄alkoxy; halogen; —OH; —CN; —COOH; —CONHR′; or R₅ and R₆ taken together may form a 5-6 membered ring;

R′ is selected from the group consisting of hydrogen, substituted or unsubstituted aliphatic, substituted or unsubstituted heteroaliphatic, substituted or unsubstituted phenyl or naphthyl, substituted or unsubstituted heteroaryl, —NH₂, —SH, or —OH.

A person of skill in the art appreciates that certain substituents may, in some embodiments, result in compounds that may have some instability and hence would be less preferred.

In some embodiments, A₁ may be selected from the group consisting of C₁-C₃alkylene optionally substituted with one, two, or three halogens, or —C(O)—.

In other embodiments, Z¹ may be

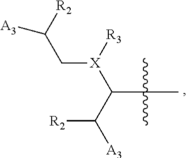

wherein R₂, independently for each occurrence, is selected from H, C₁₋₄ alkyl, or two R₁ moieties taken together form a 5- or 6-membered cycloalkyl or heterocyclic ring, wherein R₃ is H, or

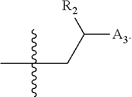

In certain embodiments, Z¹ may be

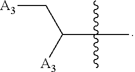

In some cases, Z¹ may be

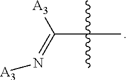

For example, in some instances, Z¹ may be

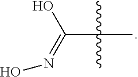 [SMILES: */C(O)=N/O]

In some embodiments, Z¹ may be a monosaccharide or a disaccharide.

In some cases, Z¹ may be selected from the group consisting of

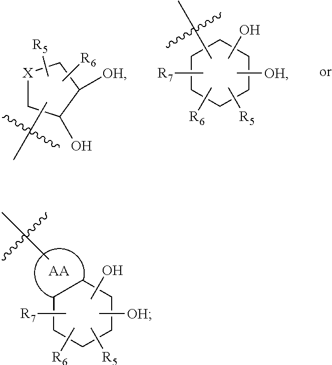

wherein

X is selected from O, S, CH, NR′, or when X is NR′, N may be covalently bonded to Y of Formula I;

R′ is selected from the group consisting of H, C₁₋₄alkyl;

R₅, R₆, and R₇ are independently selected from the group consisting of H, C₁₋₄alkyl optionally substituted by hydroxyl, amino, halo, or thio; C₁₋₄alkoxy; halogen; —OH; —CN; —COOH; —CONHR′; or a mono- or bicyclic heterocyclic optionally substituted with amino, halo, hydroxyl, oxo, or cyano; and

AA is a 5-6 membered heterocyclic ring optionally substituted by C₁₋₄alkyl optionally substituted by hydroxyl, amino, halo, or thio; C₁₋₄alkoxy; halogen; —OH; —CN; —COOH; —CONHR′, or —S—C₁₋₄alkyl. For example, in some embodiments, Z¹ may be

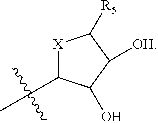

In some instances, Z¹ may be

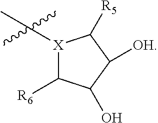

In certain cases, X may be nitrogen.

In some embodiments, Z¹ may be

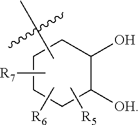

In other embodiments, Z¹ may be

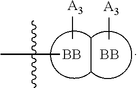

For example, in some cases, the Z¹ may be

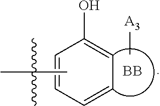

In other instances, Z¹ may be

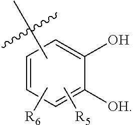

In some embodiments, Z¹ may be

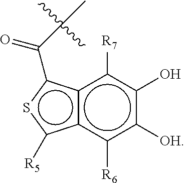

In some cases, Z¹ may be

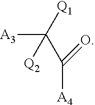

For example, Z¹ may be

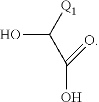

In other embodiments, Z¹ may be

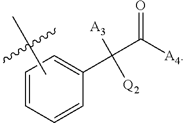

In some cases, Z¹ may be

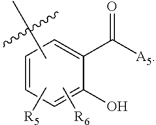

In some embodiments, Z¹ may be

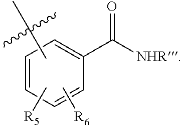

In some embodiments, Z¹ may be

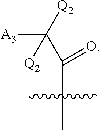

For example, Z¹ may be

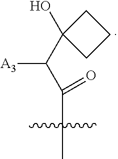

In certain embodiments, Z¹ may be

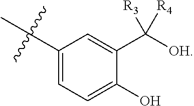

In other embodiments, Z¹ may be

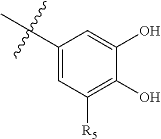

In some embodiments, the second monomer may be X²—Y²—Z² (Formula II), wherein Z² is a boronic acid or oxaborale moiety, and wherein X² is a second ligand capable of binding to a second target biomolecule segment (e.g. a segment of a fusion protein or a bromodomain of tandem bromodomains), and Y² is absent or is a connector moiety covalently bound to X² and Z². In some instances, X¹ and X² may be the same. In other instances, X¹ and X² may be different.

In some embodiments, the second monomer may be X⁴—Y⁴—Z⁴ (Formula IV), wherein Z⁴ is a boronic acid or oxaborale moiety, and wherein X⁴ is a second ligand moiety capable of binding to a protein domain, wherein the protein domain is within e.g., about 50 \acute{Å} of the bromodomain (e.g. a segment of a fusion protein or a second bromodomain of tandem bromodomains), and Y⁴ is absent or is a connector moiety covalently bound to X⁴ and Z⁴. For example, X¹ may be capable of binding to a first bromodomain, and X⁴ may be capable of binding to a second bromodomain, wherein the second bromodomain is within, e.g., about 50 \acute{Å} of the first bromodomain. In some instances, X¹ and X⁴ may be the same. In other instances, X¹ and X⁴ may be different.

In some cases, the first target biomolecule and the second target biomolecule may be different. In other embodiments, the first target biomolecule and the second target biomolecule may be the same.

In some embodiments, the linker of the second monomer, for example, Z² or Z⁴, may be selected from the group consisting of:

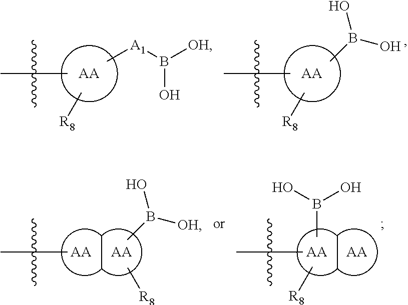

wherein

R₈ is selected from the group consisting of H, halogen, oxo, C₁₋₄alkyl optionally substituted by hydroxyl, amino, halo or thio; C₂₋₄alkenyl, C₁₋₄alkoxy; —S—C₁₋₄alkyl; —CN; —COOH; or —CONHR′;

A₁ is (a) absent; or (b) selected from the group consisting of acyl, substituted or unsubstituted aliphatic, or substituted or unsubstituted heteroaliphatic;

AA, independently for each occurrence, is phenyl, naphthyl, or a 5-7 membered heterocyclic or heteroaryl ring having one, two, or three heteroatoms, wherein AA is optionally substituted by one, two, or three substituents selected from the group consisting of halogen, C₁-4alkyl optionally substituted by hydroxyl, amino, halogen, or thio; C₂₋₄alkenyl, C₁₋₄alkoxy; —S—C₁₋₄alkyl; —CN; —COOH; —CONHR′; or two substituents together with the atoms to which they are attached form a fused 4-6 membered cycloalkyl or heterocyclic bicyclic ring system; and R′ is H or C₁₋₄alkyl.

A person of skill in the art appreciates that certain substituents may, in some embodiments, result in compounds that may have some instability and hence would be less preferred.

In certain embodiments, R₈ and the substituent comprising boronic acid may be ortho to each other, and R₈ may be —CH₂NH₂. In some cases, the linker of the second monomer may be selected from the group consisting of:

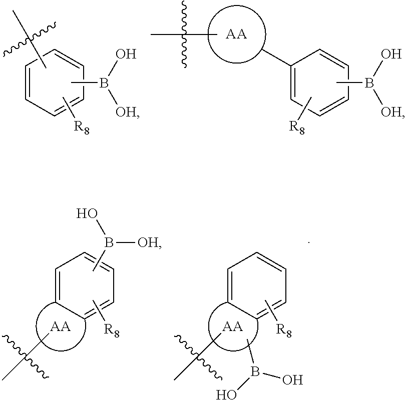

In some embodiments, the linker of the second monomer may be selected from the group consisting of:

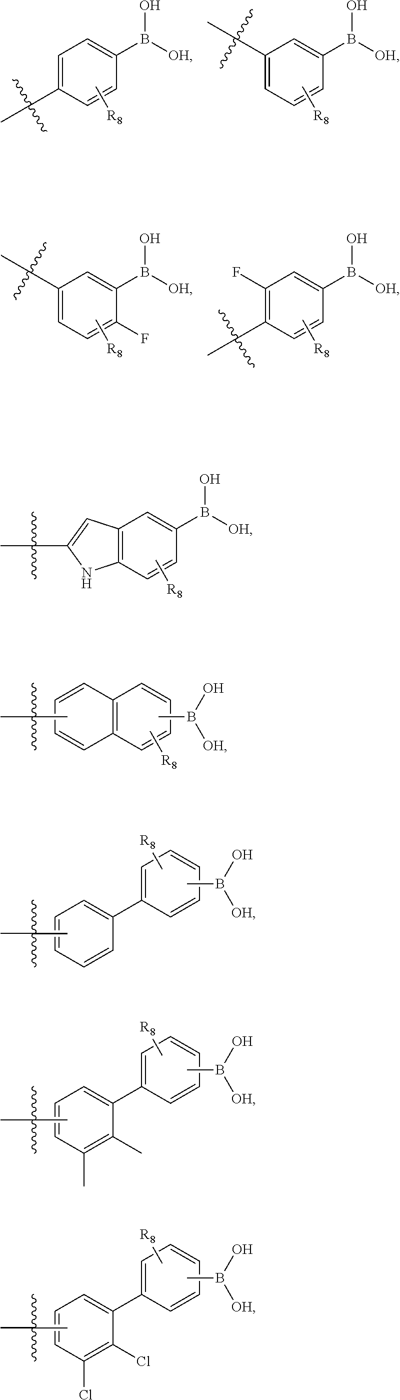

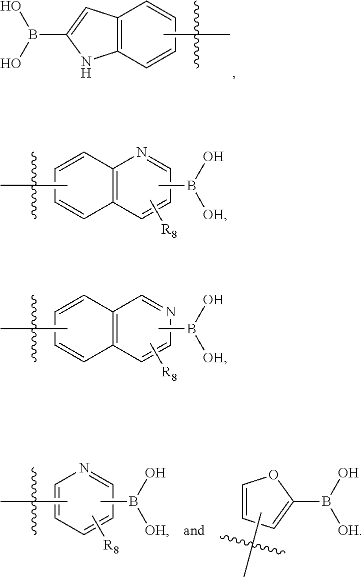

In some cases, the linker of the second monomer may be selected from the group consisting of:

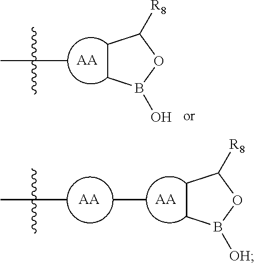

wherein

R₈ is selected from the group consisting of H, halogen, oxo, C₁₋₄alkyl optionally substituted by hydroxyl, amino, halo or thio; C₂₋₄alkenyl, C₁₋₄alkoxy; —S—C₁₋₄alkyl; —CN; —COOH; or —CONHR′;

AA, independently for each occurrence, is a 5-7 membered heterocyclic ring having one, two, or three heteroatoms, or phenyl, wherein AA is optionally substituted by one, two, or three substituents selected from the group consisting of halo, C₁₋₄alkyl optionally substituted by hydroxyl, amino, halo, or thio; C₂₋₄alkenyl, C₁₋₄alkoxy; —S—C₁₋₄alkyl; —CN; —COOH; —CONHR′; or two substituents together with the atoms to which they are attached form a fused 4-6 membered cycloalkyl or heterocyclic bicyclic ring system; and

R′ is H or C₁₋₄alkyl.

In another embodiment, a monomer may be represented by the formula: X³—Y³—Z³ (Formula III), wherein Z³ is independently selected from the group consisting of:

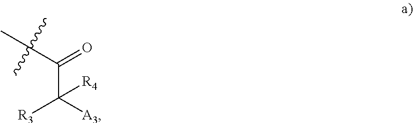

wherein

A₃ is —OH, —SH, or —NHR′;

R₃ is selected from the group consisting of H, halo, C₁₋₄alkyl, C₃₋₆cycloalkyl, and heterocycle, wherein C₁₋₄ alkyl, C₃₋₆cycloalkyl, or heterocycle may be optionally substituted by one, two, or three substituents selected from the group consisting of halo, cyano, amino, or hydroxyl; and

R₄ is selected from the group consisting of H, halo, C₁₋₄alkyl, C₃₋₆cycloalkyl, and heterocycle, wherein C₁₋₄ alkyl, C₃₋₆cycloalkyl, or heterocycle may be optionally substituted by one, two, or three substituents selected from the group consisting of halo, cyano, amino, or hydroxyl; or

R₃ and R₄ can be taken together with the atoms to which they are attached to form a substituted or unsubstituted phenyl, substituted or unsubstituted C₃₋₆cycloalkyl, substituted or unsubstituted heteroaryl or substituted or unsubstituted saturated heterocycle;

R′ is H or C₁₋₄alkyl; and

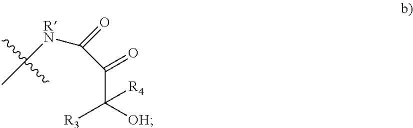

wherein

R′ is C₁₋₄alkyl optionally substituted with hydroxyl; —NH₂; —OH; and C₁₋₄alkoxy;

R₃ is selected from the group consisting of H, halo, C₁₋₄alkyl, C₃₋₆cycloalkyl and heterocycle, wherein C₁₋₄alkyl, C₃₋₆cycloalkyl, or heterocycle may be optionally substituted by one, two, or three substituents selected from the group consisting of halo, cyano, amino, or hydroxyl;

R₄ is selected from the group consisting of H, C₁₋₄alkyl, C₃₋₆cycloalkyl and heterocycle, wherein C₁₋₄alkyl, C₃₋₆cycloalkyl, or heterocycle may be optionally substituted by one, two or three substituents selected from the group consisting of halo, cyano, amino, or hydroxyl; or

R₃ and R₄ can be taken together with the atoms to which they are attached to form a substituted or unsubstituted phenyl, substituted or unsubstituted C₃₋₆cycloalkyl, substituted or unsubstituted heteroaryl or substituted or unsubstituted saturated heterocycle; and

wherein Z³ is a linker moiety capable of binding to one or more X³—Y³—Z³ monomers to form a biologically useful multimer.

A person of skill in the art appreciates that certain substituents may, in some embodiments, result in compounds that may have some instability and hence would be less preferred.

In another embodiment, silyl monomers are contemplated that are capable of forming a biologically useful multimer when in contact with one, two, three or more second silyl monomers in an aqueous media. The silyl monomers can be represented by Formula III above, (e.g., X³—Y³—Z³), but wherein Z³ is independently selected from the group consisting of:

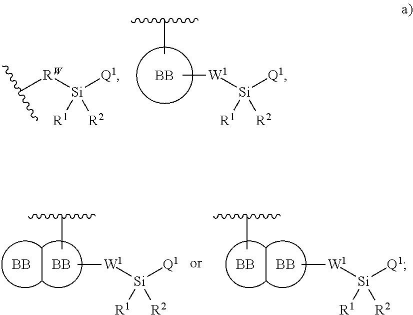

wherein

R^{W} is selected from the group consisting of —C₁₋₄alkyl-, —O—C₁₋₄alkyl-, —N(R^{a})—, —N(R^{a})—C₁₋₄alkyl-, —O—, —C(O)C₁₋₄ alkyl-, —C(O)—O—C₁₋₄ alkyl-, —C₂₋₆ alkenyl-, —C₂₋₆ alkynyl-, —C₃₋₆ cycloalkyl-, -phenyl- and -heterocycle-; wherein C₁₋₄alkyl, R^{a}, R^{b}, C₂₋₆alkenyl, C₂₋₆alkynyl, C₃₋₆cycloalkyl, phenyl and heteroaryl may be optionally substituted by one, two, three or more substituents selected from the group consisting of C₁₋₄alkyl, C₁₋₄alkoxy, —C(O)C₁₋₄alkyl, —C(O)—O—C₁₋₄alkyl, —C(O)—NR^{a}R^{b}, halogen, cyano, hydroxyl, phenyl, R^{a} and R^{b};

W¹, independently for each occurrence, is (a) absent; or (b) selected from the group consisting of —C₁₋₄ alkyl-, —O—C₁₋₄ alkyl-, —C(O)—C₁₋₄ alkyl-, —N(R^{a})—C₁₋₄ alkyl-, —C(O)—O—C₁₋₄ alkyl-, —C₂₋₆alkenyl-, —C₂₋₆alkynyl-, —C₃₋₆cycloalkyl-, -phenyl- or -heteroaryl-; wherein C₁₋₄alkyl, C₂-6alkenyl, C₂₋₆alkynyl, C₃₋₆cycloalkyl, R′, phenyl and heteroaryl are optionally substituted independently, for each occurrence, with one, two, three or more substituents selected from the group consisting of C₁₋₄ alkyl, C₁₋₄alkoxy, —C(O)C₁₋₆ alkyl, —C(O)—O—C₁₋₄ alkyl, halogen, hydroxyl, nitro and cyano;

R′ is independently selected, for each occurrence, from the group consisting of hydrogen, substituted or unsubstituted aliphatic, and substituted or unsubstituted heteroaliphatic;

Q¹ is independently selected, for each occurrence, from the group consisting of —NHR′, —SH, —OH, —O—C₁₋₆ alkyl, —S—C₁₋₆alkyl, phenoxy, —S-phenyl, heteroaryl, —O-heteroaryl, —S-heteroaryl, halogen and —O—C₁₋₆alkyl-NR^{a}R^{b};

R^{a} and R^{b} are independently selected, for each occurrence, from the group consisting of hydrogen and C₁₋₄alkyl; wherein C₁₋₄alkyl may be optionally substituted by one or more substituents selected from the group consisting of halogen, cyano, oxo and hydroxyl; or

R^{a} and R^{b}, together with the nitrogen to which they are attached, may form a 4-7 membered heterocyclic ring, which may have an additional heteroatom selected from O, S, or N; wherein the 4-7 membered heterocyclic ring may be optionally substituted by one or more substituents selected from the group consisting of halogen, cyano, oxo and hydroxyl;

R¹ and R² are selected independently, for each occurrence, from the group consisting of —OH, C₁₋₆alkyl, —O—C₁₋₆alkyl, C₂₋₆alkenyl, C₃₋₆cycloalkyl, —C₁₋₆alkyl-NR^{a}R^{b}, phenyl and heteroaryl; wherein C₁₋₆alkyl, C₂₋₆alkenyl, C₃₋₆cycloalkyl, R^{a}, R^{b}, phenyl and heteroaryl, independently selected, for each occurrence, may be optionally substituted by one or more substituents selected from the group consisting of halogen, cyano, hydroxyl, C₁₋₆alkyl, and phenyl;

BB, independently for each occurrence, is a 4-7-membered cycloalkyl, heterocyclic, phenyl, naphthyl, or heteroaryl moiety, wherein the cycloalkyl, heterocyclic, phenyl, naphthyl, or heteroaryl moiety is optionally substituted with one, two, three or more groups represented by R^{BB}; wherein R¹, independently for each occurrence, may be optionally bonded to BB;

each R^{BB} is independently selected, for each occurrence, from the group consisting of hydrogen, halogen, nitro, cyano, hydroxyl, amino, thio, —COOH, —CONHR′, substituted or unsubstituted aliphatic, and substituted or unsubstituted heteroaliphatic; or two R^{BB} together with the atoms to which they are attached form a fused 5- or 6-membered cycloalkyl or heterocyclic bicyclic ring system; and

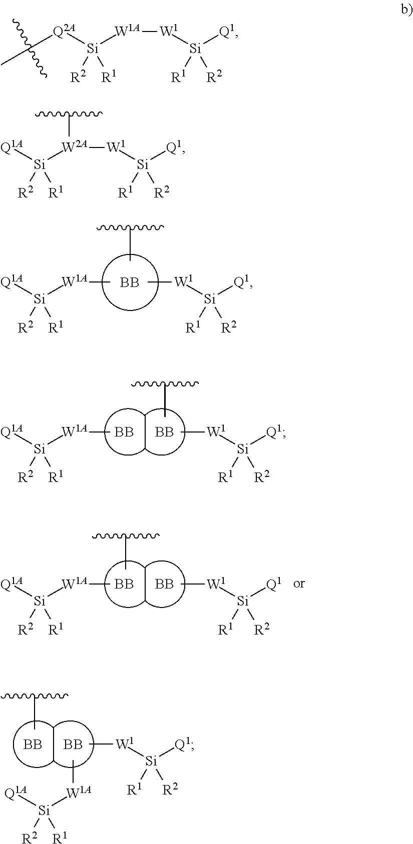

wherein

Q^{2A} is selected from the group consisting of —NH—, —S—, —O—, —O—C₁₋₆ alkyl-, —C₁₋₆ alkyl-O—, —N(R′)—C₁₋₆alkyl-, —C₁₋₆alkyl-N(R′)—, —S—C₁₋₆ alkyl-, —C₁₋₆ alkyl-S— and —O—C₁₋₆ alkyl-NR^{a}—

W¹ and W^{1A}, independently for each occurrence, are (a) absent; or (b) selected from the group consisting of —O—, —C₁₋₄alkyl-, —O—C₁₋₄alkyl-, —N(R^{a})—C₁₋₄alkyl-, —C(O)C₁₋₄alkyl-, —C(O)—O—C₁₋₄alkyl-, —C₂₋₆alkenyl-, —C₂₋₆alkynyl-, —C₃₋₆cycloalkyl-, -phenyl- and -heteroaryl-; wherein C₁₋₄alkyl, C₂₋₆alkenyl, C₂₋₆alkynyl, C₃₋₆cycloalkyl, R′, phenyl and heteroaryl may be optionally substituted independently, for each occurrence, with one, two, three or more substituents selected from the group consisting of C₁₋₄alkyl, C₁₋₄alkoxy, —C(O)C₁₋₆ alkyl, —C(O)—O—C₁₋₄ alkyl, halogen, hydroxyl, nitro and cyano;

R′ is independently selected, for each occurrence, from the group consisting of hydrogen, substituted or unsubstituted aliphatic, and substituted or unsubstituted heteroaliphatic;

Q¹ and Q^{1A} are independently selected, for each occurrence, from the group consisting of —NHR′, —SH, —OH, —O—C₁₋₆alkyl, —S—C₁₋₆alkyl, phenoxy, —S-phenyl, heteroaryl, —O-heteroaryl, —S-heteroaryl, halogen and —O—C₁₋₆alkyl-NR^{a}R^{b};

R^{a} and R^{b} are independently selected, for each occurrence, from the group consisting of hydrogen and C₁₋₄alkyl; wherein C₁₋₄alkyl may be optionally substituted by one or more substituents selected from the group consisting of halogen, cyano, oxo and hydroxyl; or

R^{a} and R^{b}, together with the nitrogen to which they are attached, may form a 4-7 membered heterocyclic ring, which may have an additional heteroatom selected from O, S, or N; wherein the 4-7 membered heterocyclic ring may be optionally substituted by one or more substituents selected from the group consisting of halogen, cyano, oxo and hydroxyl;

R¹ and R² are selected independently, for each occurrence, from the group consisting of —OH, C₁₋₆alkyl, —O—C₁₋₆alkyl, C₂₋₆alkenyl, C₃₋₆cycloalkyl, —C₁₋₆alkyl-NR^{a}R^{b}, phenyl and heteroaryl; wherein C₁₋₆alkyl, C₂₋₆alkenyl, C₃₋₆cycloalkyl, R^{a}, R^{b}, phenyl and heteroaryl, independently selected, for each occurrence, may be optionally substituted by one or more substituents selected from the group consisting of halogen, cyano, hydroxyl, C₁₋₆alkyl, and phenyl;

W^{2A} is selected from the group consisting of N and CR^{W2A}.

R^{W2A} is selected from the group consisting of hydrogen, C₁₋₄alkyl, —O—C₁₋₄alkyl, C₂₋₆alkenyl, C₂₋₆alkynyl, C₃₋₆cycloalkyl, phenyl and heteroaryl; wherein C₁₋₄alkyl, C₂₋₆alkenyl, C₂₋₆alkynyl, C₃₋₆cycloalkyl, phenyl and heteroaryl may be optionally substituted independently, for each occurrence, with one, two, three or more substituents selected from the group consisting of halogen, hydroxyl and cyano;

BB, independently for each occurrence, is a 4-7-membered cycloalkyl, heterocyclic, phenyl, naphthyl, or heteroaryl moiety; wherein the cycloalkyl, heterocyclic, phenyl, naphthyl, or heteroaryl moiety may be optionally substituted with one, two, three or more groups represented by R^{BB}; wherein R¹, independently for each occurrence, may be optionally bonded to BB;

each R^{BB} is independently selected, for each occurrence, from the group consisting of hydrogen, halogen, nitro, cyano, hydroxyl, amino, thio, —COOH, —CONHR′, substituted or unsubstituted aliphatic, substituted or unsubstituted heteroaliphatic; or two R^{BB} together with the atoms to which they are attached may form a fused 5- or 6-membered cycloalkyl or heterocyclic bicyclic ring system.

A person of skill in the art appreciates that certain substituents may, in some embodiments, result in compounds that may have some instability and hence would be less preferred.

As discussed above, a monomer may be capable of reacting with one or more other monomers to form a multimer. In some embodiments, a first monomer may react with a second monomer to form a dimer. In other embodiments, a first monomer may react with a second monomer and a third monomer to form a trimer. In still other embodiments, a first monomer may react with a second monomer, a third monomer, and a fourth monomer to form a tetramer. In some embodiments, each of the monomers that form a multimer may be essentially the same. In some embodiments, each of the monomers that form a multimer may be substantially different. In certain embodiments, at least some of the monomers that form a multimer may be essentially the same or may be substantially different.

In some embodiments, the linker element of a first monomer and the linker element of a second monomer may be substantially different. In other embodiments, a connector element of a first monomer and a connector element of a second monomer may be substantially different. In still other embodiments, the ligand moiety (e.g., a pharmacophore) of a first monomer and the ligand moiety (e.g., a pharmacophore) of the second monomer may be substantially different.

In some cases, formation of a multimer from a plurality of monomers may be irreversible. In some embodiments, formation of a multimer from a plurality of monomers may be reversible. For example, in some embodiments, the multimer may have an oligomer or dimer dissociation constant between 10 mM and 1 nM, in some embodiments between 1 mM and 100 nM, in some embodiments between 1 mM and 1 and in some embodiments between 500 and 1 In certain embodiments, the multimer may have a dissociation constant of less than 10 mM, in some embodiments less than 1 mM, in some embodiments less than 500 in some embodiments less than 100 in some embodiments less than 50 in some embodiments less than 1 in some embodiments less than 100 nM, and in some embodiments less than 1 nM.

### B) Ligands

The ligand moieties X¹, X², X³ and X⁴ of Formulas I, II, III and IV may, in some embodiments, be the same or different. For example, ligand moieties are independently contemplated herein.

In one embodiment, the ligand moiety may be a pharmacophore. A pharmacophore is typically an arrangement of the substituents of a moiety that confers biochemical or pharmacological effects. In some embodiments, identification of a pharmacophore may be facilitated by knowing the structure of the ligand in association with a target biomolecule. In some cases, pharmacophores may be moieties derived from molecules previously known to bind to target biomolecules (e.g., proteins), fragments identified, for example, through NMR or crystallographic screening efforts, molecules that have been discovered to bind to target proteins after performing high-throughput screening of natural products libraries, previously synthesized commercial or non-commercial combinatorial compound libraries, or molecules that are discovered to bind to target proteins by screening of newly synthesized combinatorial libraries. Since most pre-existing combinatorial libraries are limited in the structural space and diversity that they encompass, newly synthesized combinatorial libraries may include molecules that are based on a variety of scaffolds.

In one embodiment, monomers that include a pharmacophore may bind to a bromodomain. Such monomers may form a multimer, as disclosed herein, that may be capable of binding to tandem bromodomains, e.g. within a BET family of bromodomains that contain tandem bromodomains in close proximity, making them capable of binding two acetylated lysine residues with greater specificity. For example, a “BET bromodomain” may refer to the bromodomains in BRD2, BRD3, BRD4 or BRD-t. A person skilled in the art may appreciate that additional pharmacophores may be discovered in the future and that the pharmacophores illustrated herein are not intended to limit in any way the claims.

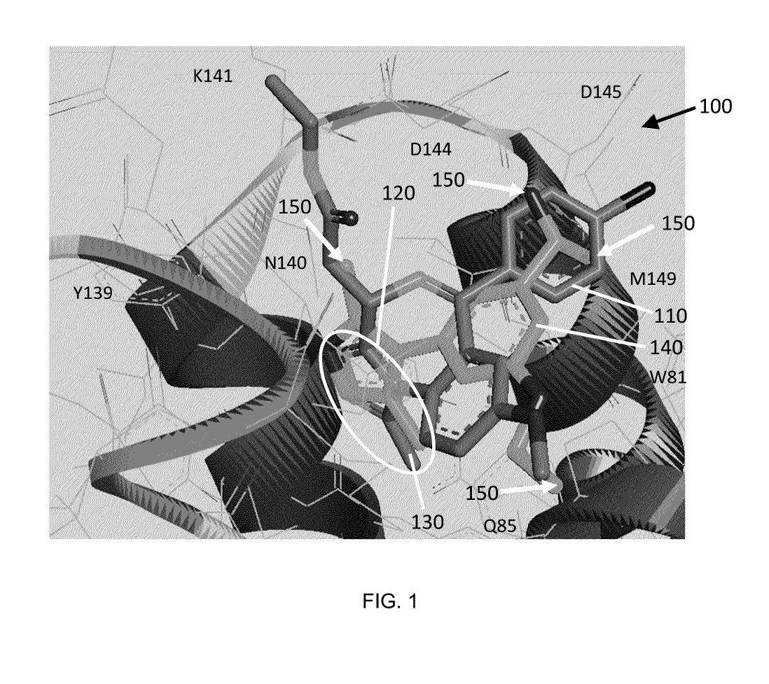

In some embodiments, a ligand (e.g., a pharmacophore) may have one or more preferred attachment points for connecting the pharmacophore to the linker (e.g., with or without a connector moiety). In certain embodiments, an attachment point on a pharmacophore may be chosen so as to preserve at least some ability of the pharmacophore to bind to a bromodomain. In one embodiment, preferred attachment points may be identified using X-ray crystallography. The following description of a non-limiting exemplary method illustrates how a preferred attachment point may be identified. For example, as shown in FIG. 1, using the 3P5O structure 100 from the protein databank (PDB), a small molecule 110 (dark gray) labeled “EAM1” in the PDB file [also known as I-BET or IBET762] may be identified. The I-BET triazolo ring (indicated by white circle 120) contains two adjacent nitrogen atoms in the 3 and 4 positions and a methyl group 130 bound to the adjacent carbon at the 5 position. Together, the nitrogen atoms and methyl group constitute an acetyl lysine mimetic. The corresponding acetyl lysine mimetic in the new pharmacophore 140 (light gray) should be aligned to these elements. The final conformation and orientation of the newly aligned pharmacophore 140 in the site may be determined using a variety of approaches known to computational chemists, but can be done as simply as performing an energy minimization using a molecular mechanics forcefield. It should be noted that the alphanumeric identifiers in FIG. 1 (e.g., K141, D144, M149, etc.) correspond to amino acid residues in the 3P5O structure, where the letter of the identifier is the one-letter amino acid symbol and the number of the identifier is the position of the amino acid residue in the primary sequence of the protein. Attachment points 150 on the aligned pharmacophore which permit access to amino acid residues D96, Y139, N140, K141, D144, D145, M149, W81, or Q85 in the 3P5O structure are considered preferred attachment points for linkers. It should be apparent to those skilled in the art that overlays of the I-BET pharmacophore with other alternate pharmacophores can be used to identify potential attachment points.

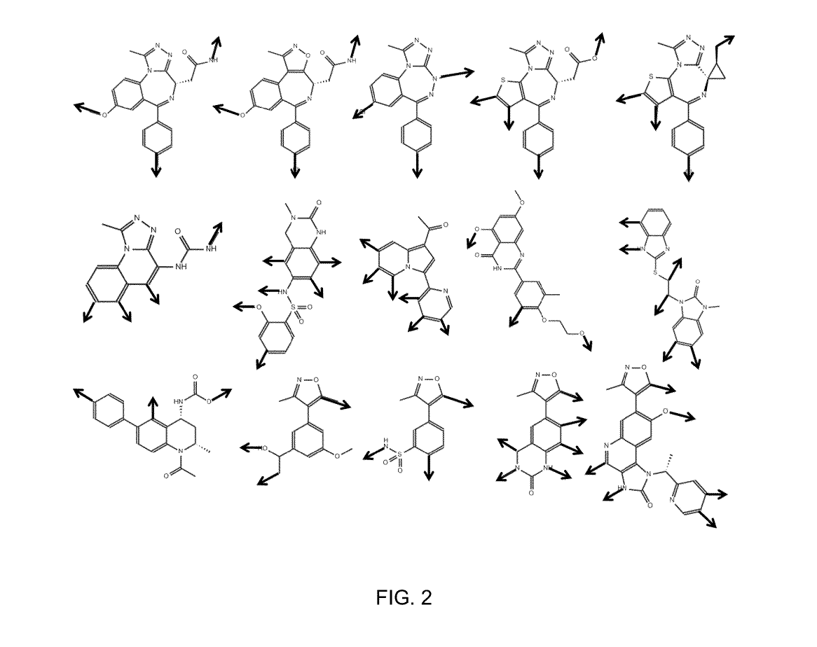

FIG. 2 provides a non-limiting set of pharmacophores (i.e., ligands) showing preferred attachment points (indicated by circled arrows) for connecting the pharmacophore to a linker.

In one embodiment, X¹ is a first ligand moiety capable of binding to a first bromodomain. In another embodiment X² is a second ligand moiety capable of binding to a second bromodomain, or to another domain, e.g., near or adjacent to the first bromodomain.

For example, the disclosed ligand moieties, X¹, X², X³ and X⁴ of Formulas I, II, III and IV may be or include bromodomain ligands as described herein. It will be appreciated that the ligands disclosed herein can be attached at any open site to a —Y—Z moiety (e.g., —Y¹—Z¹, —Y²—X², —Y³—Z³, and —Y⁴—Z⁴) as described herein. Such embodiments described below include specific references to each attachment site. Exemplary bromodomain ligands include quinolines represented by the structures:

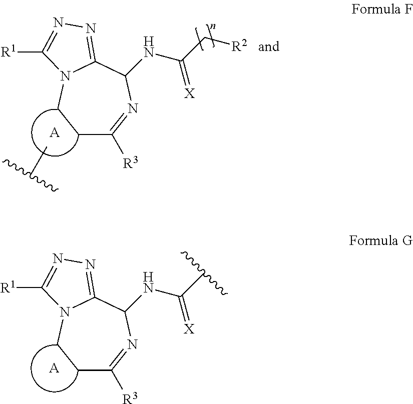

wherein:

X is O or S;

R¹ is C₁₋₆alkyl, haloC₁₋₆alkyl, —(CH₂)—OR^{1a}, or —(CH₂)ₘNR^{1b}R^{1c}; wherein R^{1a} is hydrogen, C₁₋₆alkyl or haloC₁₋₆alkyl; R^{1b} and R^{1c}, which may be the same or different, are hydrogen, C₁₋₆alkyl or haloC₁₋₆alkyl; and m and n, which may be the same or different, are 1, 2 or 3;

R² is R^{2a}, —OR^{2b}, or —NR^{2c}R^{2d}; wherein R²a and R^{2b} are carbocyclyl, carbocyclylC₁₋₄alkyl, heterocyclyl or heterocyclylC₁₋₄alkyl, or R^{2a} is carbocyclylethenyl or heterocyclylethenyl, wherein any of the carbocyclyl or heterocyclyl groups defined for R^{2a} or R^{2b} are optionally substituted by one or more groups independently selected from the group consisting of halogen, C₁₋₆ alkyl, haloC₁₋₆alkyl, C₁₋₆ alkoxy, haloC₁₋₆ alkoxy, nitro, cyano, dimethylamino, benzoyl and azido; or two adjacent groups on any of the carbocyclyl or heterocyclyl groups defined for R^{2a} or R^{2b} together with the interconnecting atoms form a 5 or 6-membered ring which ring may contain 1 or 2 heteroatoms independently selected from the group consisting of O, S and N; or

R²a and R^{2b} are C₁₋₆alkyl or haloC₁₋₆alkyl; and R^{2c} and R^{2d}, which may be the same or different, are carbocyclyl, carbocyclylC₁₋₄alkyl, heterocyclyl or heterocyclylC₁₋₄alkyl, wherein any of the carbocyclyl or heterocyclyl groups defined for R^{2c} or R^{2d} are optionally substituted by one or more groups independently selected from the group consisting of halogen, C₁₋₆alkyl, haloC₁₋₆alkyl, C₁₋₆ alkoxy, haloC₁₋₆ alkoxy, nitro, cyano and —CO₂C₁₋₄ alkyl; or two adjacent groups on any of the carbocyclyl or heterocyclyl groups defined for R^{2c} and R^{2d} together with the interconnecting atoms form a 5 or 6-membered ring which ring may contain 1 or 2 heteroatoms independently selected from the group consisting of O, S and N; or

R^{2c} and R^{2d} are independently hydrogen, C₁₋₆alkyl or haloC₁₋₆alkyl;

R³ is C₁₋₆alkyl, phenyl, naphthyl, heteroaryl carbocyclyl or heterocyclyl, optionally substituted independently by one or more substitutents selected from the group consisting of halogen, —SR, —S(O)R′, —NHR′, —OR′, C₁₋₆alkyl, haloC₁₋₆alkyl, C₁₋₆alkoxy, haloC₁₋₆alkoxy, nitro and cyano;

R′ is H or C₁₋₆alkyl;

A is a benzene or aromatic heterocyclic ring, each of which is optionally substituted; and

n is 0, 1 or 2.

In some embodiments, compounds of Formula F or Formula G may be selected from the group consisting of:

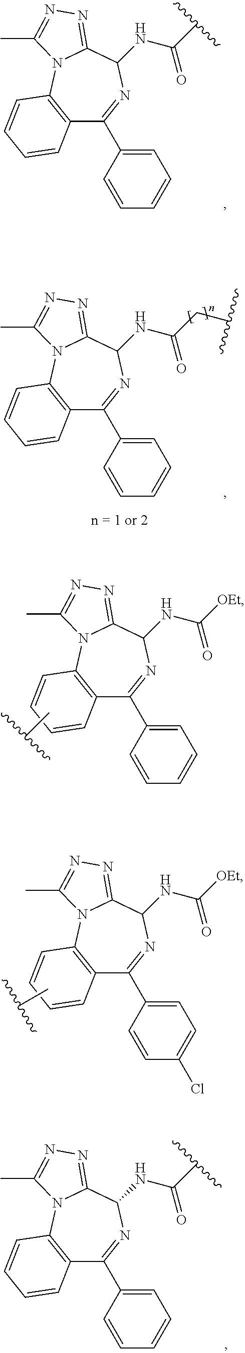

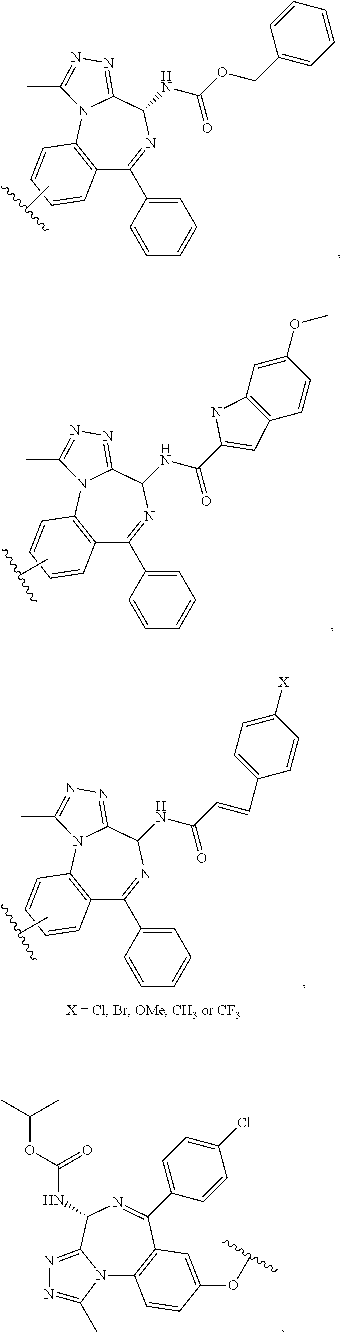

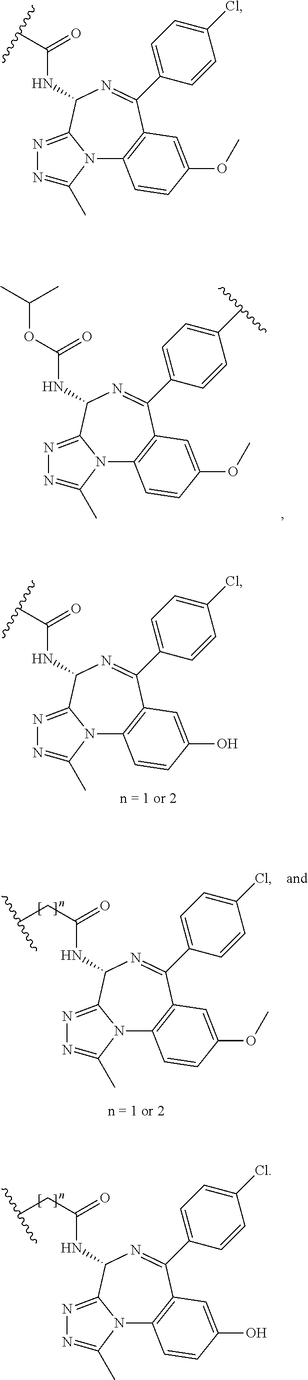

In another embodiment, exemplary bromodomain ligands include benzodiazepines represented by the structures:

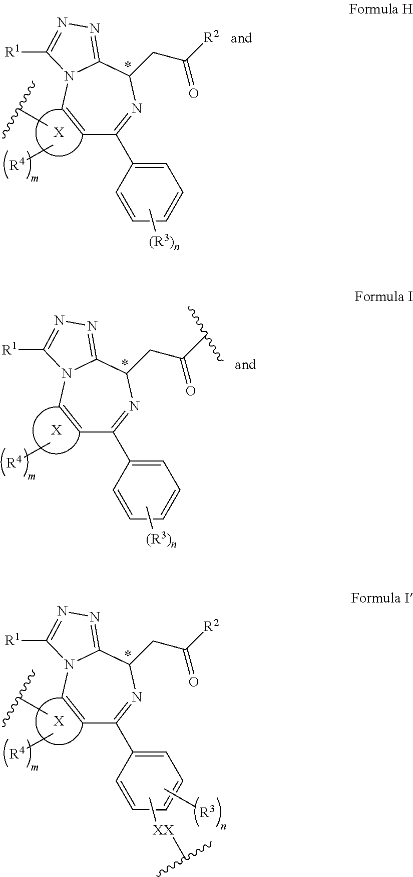

wherein:

X is phenyl, naphthyl, or heteroaryl;

R¹ is C₁₋₃ alkyl, C₁₋₃alkoxy or —S—C₁₋₃ alkyl;

R² is —NR^{2a}R^{2a′} or —OR^{2b}; wherein one of R^{2a} or R^{2a′} is hydrogen, and R^{2b} or the other of R^{2a} or R^{2a′} is selected from the group consisting of C₁₋₆alkyl, haloC₁₋₆alkyl, R^{2c}R^{2c′}N—C₂₋₆alkyl, carbocyclyl, carbocyclyloC₁₋₄alkyl, heterocyclyl and heterocyclylC₁₋₄alkyl, wherein any of the carbocyclyl or heterocyclyl groups are optionally substituted by one or more substituents selected from the group consisting of halogen, C₁₋₆alkyl, haloC₁₋₆alkyl, C₁₋₆alkoxy, haloC₁₋₆alkoxy, carbonyl, —CO-carbocyclyl, azido, amino, hydroxyl, nitro and cyano, wherein the —CO-carbocyclyl group may be optionally substituted by one or more substituents selected from the group consisting of halogen, C₁₋₆alkyl, haloC₁₋₆alkyl, C₁₋₆alkoxy, haloC₁₋₆alkoxy, azido, nitro and cyano; or

two adjacent groups on any of the carbocyclyl or heterocyclyl groups together with the interconnecting atoms form a 5- or 6-membered ring which ring may contain 1 or 2 heteroatoms independently selected from the group consisting of O, S and N; or R²a and R^{2a′} together with the N atom to which they are attached form a 4-, 5-, 6- or 7-membered ring which optionally contains 1 or 2 heteroatoms independently selected from the group consisting of O, S and N; wherein the 4-, 5-, 6 or 7-membered ring is optionally substituted by C₁₋₆alkyl, hydroxyl or amino;

R^{2c} and R^{2c′} are independently hydrogen or C₁₋₆alkyl;

each R³ is independently selected from the group consisting of hydrogen, hydroxyl, thiol, sulfinyl, sulfonyl, sulfone, sulfoxide, —OR^{t}, —NR^{t}R^{tt}, —S(O)₂NR^{t}R^{tt}, —S(O)_{w}R^{t}R^{tt} (where t and tt are independently selected from H, phenyl or C₁₋₆alkyl, and w is 0, 1, or 2), halo, C₁₋₆alkyl, haloC₁₋₆alkyl, C₁₋₆alkoxy, haloC₁₋₆alkoxy, nitro, cyano, CF₃, —OCF₃, —COOR⁵, —C₁₋₄alkylamino, phenoxy, benzoxy, and C₁₋₄alkylOH;

XX is selected from the group consisting of a bond, NR′″ (where R′″ is H, C₁₋₆alkyl or phenyl), —O—, or S(O)_{w} wherein w is 0, 1 or 2, and C₁₋₆alkyl; (and wherein in some embodiments XX is in the para position);

each R⁴ is hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆alkyl, nitro, cyano, CF₃, —OCF₃, —COOR⁵; —OS(O)₂C₁₋₄alkyl, phenyl, naphthyl, phenyloxy, benzyloxy or phenylmethoxy, wherein C₁₋₆alkyl, phenyl, and naphthyl are optionally substituted by one two or three substituents selected from the group consisting of hydroxyl, halogen, amino, nitro;

R⁵ is C₁₋₃ alkyl;

* denotes a chiral center;

m is an integer 1 to 3; and

n is an integer 1 to 5. In some embodiments, the chiral center has an S configuration.

In some embodiments, compounds of Formula H or Formula I may be selected from the group consisting of:

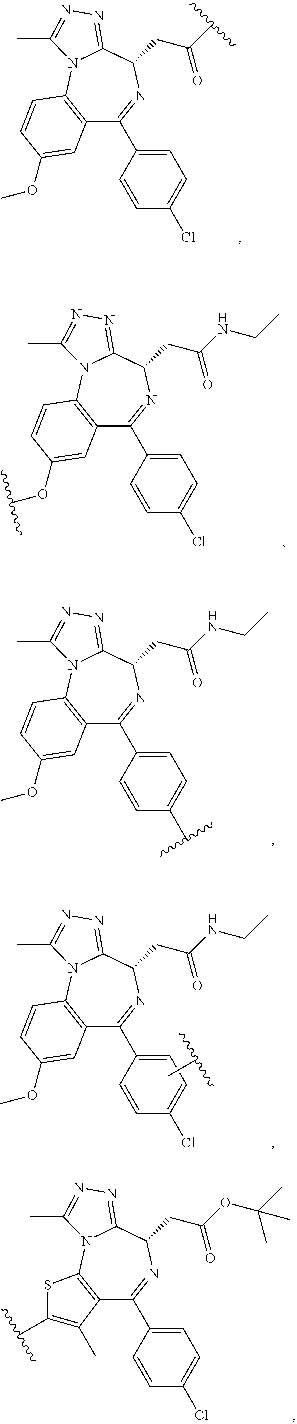

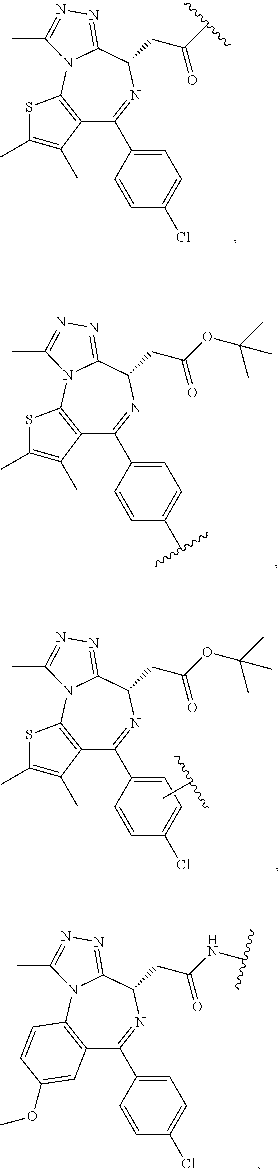

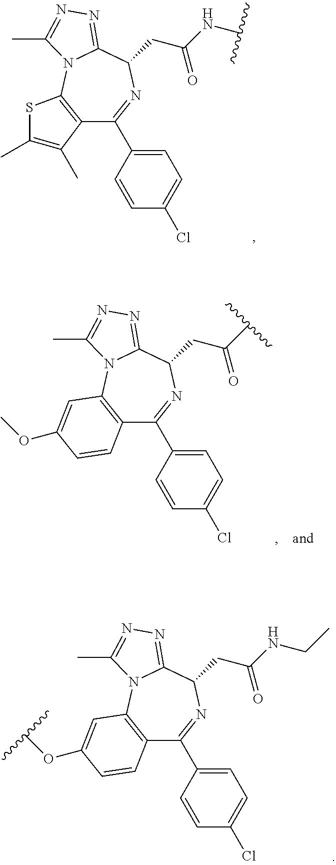 [SMILES: *NC(=O)C[C@@H]1N=C(c2ccc(Cl)cc2)c2c(sc(C)c2C)-n2c(C)nnc21 ; *C(=O)C[C@@H]1N=C(c2ccc(Cl)cc2)c2ccc(OC)cc2-n2c(C)nnc21 ; *Oc1ccc2c(c1)-n1c(C)nnc1[C@H](CC(=O)NCC)N=C2c1ccc(Cl)cc1]

For example, compounds of Formula F, Formula G, Formula H or Formula I may be selected from the group consisting of:

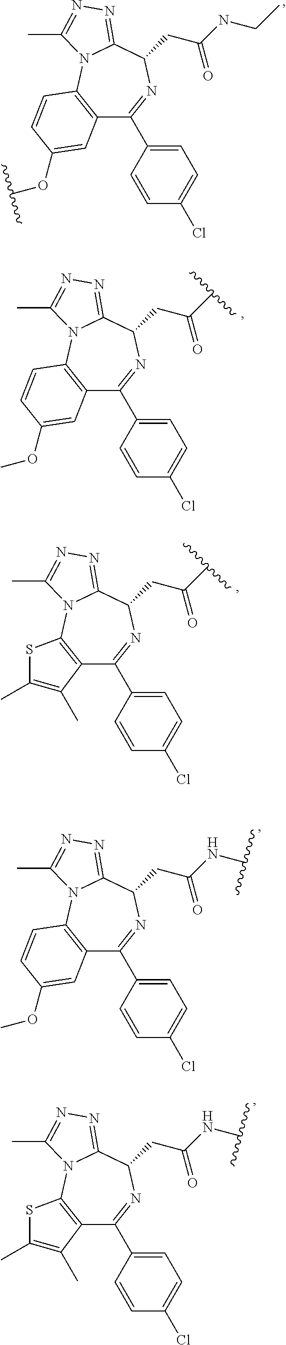

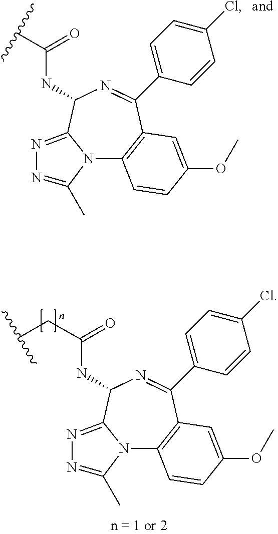

In some embodiments, exemplary bromodomain ligands include compounds represented by the structures:

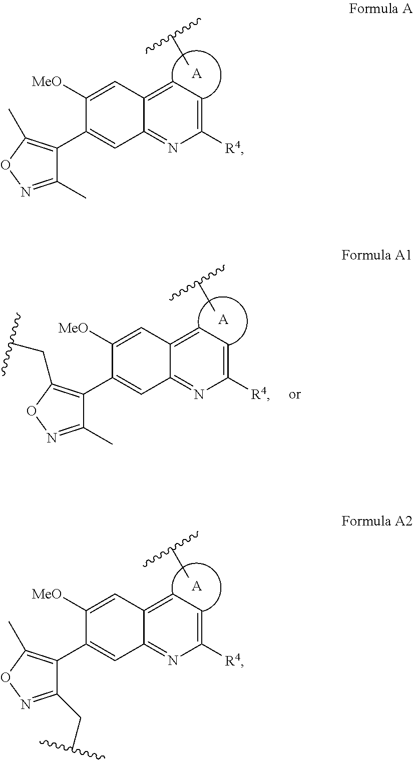

wherein:

R⁴ is hydrogen, cyano or C₁₋₆ alkyl;

A is selected from the group consisting of:

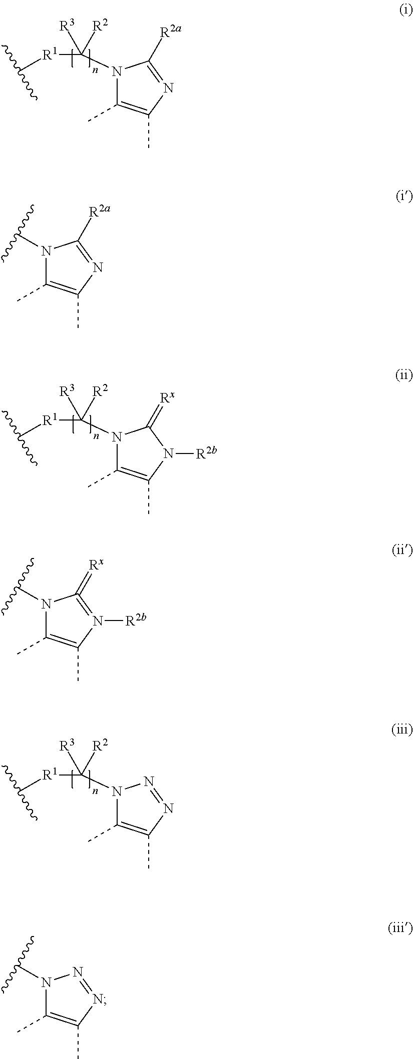

R^{x} is O, NR^{2a}, or S;

R¹ is C₁₋₆alkyl, C₃₋₆cycloalkyl, a 5 or 6 membered heterocyclyl, an aromatic group or a heteroaromatic group, wherein the aromatic group or the heteroaromatic group is optionally substituted by one to three groups selected from the group consisting of halogen, hydroxy, cyano, nitro, C₁₋₆ alkyl, C₁₋₄ alkoxy, haloC₁₋₄ alkyl, haloC₁₋₄ alkoxy, hydroxyC₁₋₄ alkyl, C₁₋₄ alkoxy C₁₋₄ alkyl, C₁₋₄ alkoxycarbonyl, C₁₋₄ alkylsulfonyl, C₁₋₄ alkylsulfonyloxy, C₁₋₄ alkylsulfonyl C₁₋₄alkyl and C₁₋₄ alkylsulfonamido;

R² is hydrogen or C₁₋₆alkyl;

R^{2a} is selected from the group consisting of H, C₁₋₆alkyl, C₁₋₆haloalkyl, (CH₂)ₘcyano, (CH₂)ₘOH, (CH₂)ₘC₁₋₆alkoxy, (CH₂)ₘC₁₋₆haloalkoxy, (CH₂)ₘC₁₋₆haloalkyl, (CH₂)ₘC(O)NR^{a}R^{b}, (CH₂)ₘNR^{a}R^{b} and (CH₂)ₘ C(O)CH₃, (CHR⁶)ₚphenyl optionally substituted by C₁₋₆alkyl, C₁₋₆alkoxy, cyano, halo C₁₋₄alkoxy, haloC₁₋₄alkyl, (CHR⁶)ₚheteroaromatic, (CHR⁶)ₚheterocyclyl; wherein R^{a} is H, C₁₋₆alkyl, or heterocyclyl; wherein R^{b} is H or C₁₋₆alkyl, or

R^{a} and R^{b} together with the N to which they are attached form a 5 or 6 membered heterocyclyl;

R^{2b} is H, C₁₋₆alkyl, (CH₂)₂C₁₋₆alkoxy, (CH₂)₂cyano, (CH₂)ₘphenyl or (CH₂)₂heterocyclyl;

R³ is hydrogen;

R⁶ is hydrogen or C₁₋₆alkyl;

m is 0, 1, 2 or 3;

n is 0, 1 or 2; and

p is 0, 1 or 2.

In some embodiments, compounds of Formulae A, A1, and A2 may be selected from the group consisting of:

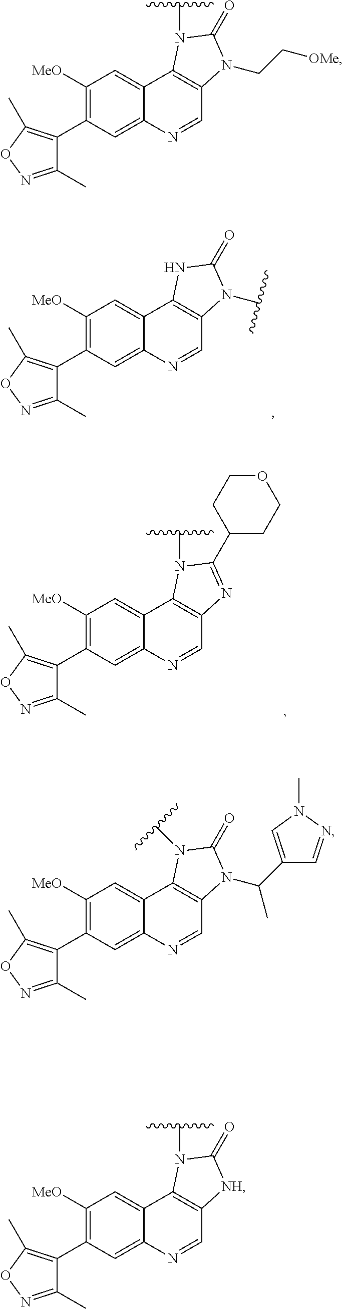 [SMILES: *n1c(=O)n(CCOC)c2cnc3cc(-c4c(C)noc4C)c(OC)cc3c21 ; *n1c(=O)[nH]c2c3cc(OC)c(-c4c(C)noc4C)cc3ncc21 ; *n1c(C2CCOCC2)nc2cnc3cc(-c4c(C)noc4C)c(OC)cc3c21 ; *n1c(=O)n(C(C)c2cnn(C)c2)c2cnc3cc(-c4c(C)noc4C)c(OC)cc3c21 ; *n1c(=O)[nH]c2cnc3cc(-c4c(C)noc4C)c(OC)cc3c21]

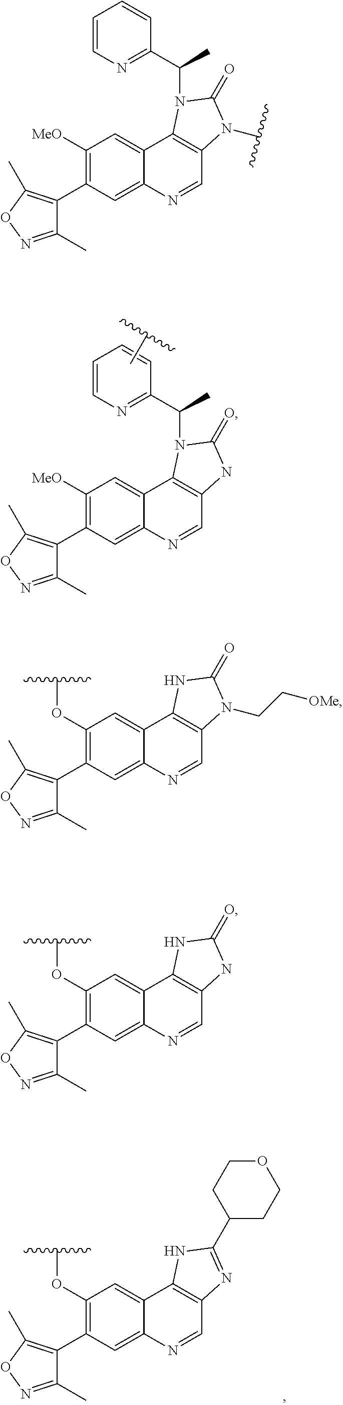

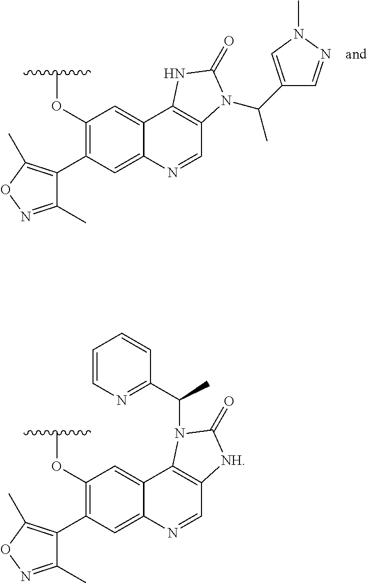 [SMILES: *Oc1cc2c(cc1-c1c(C)noc1C)ncc1c2[nH]c(=O)n1C(C)c1cnn(C)c1 ; *Oc1cc2c(cc1-c1c(C)noc1C)ncc1[nH]c(=O)n([C@H](C)c3ccccn3)c12]

In another embodiment, exemplary bromodomain ligands include tetrahydroquinolines represented by the structures:

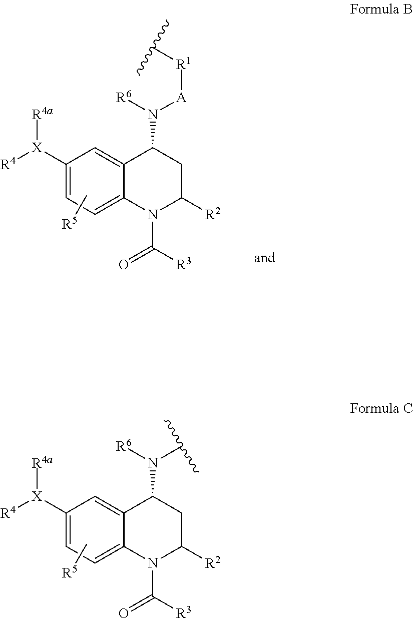

wherein:

A is a bond, C₁₋₄alkyl or —C(O)—;

X is:
i) a 6 to 10 membered aromatic group, or
ii) a 5 to 10 membered heteroaromatic comprising 1, 2 or 3 heteroatoms selected from the group consisting of O, N and S;

R¹ is:
i) phenyl optionally substituted by 1 or 2 substituents independently selected from the group consisting of halogen, cyano, C₁₋₆ alkyl, C₁₋₆haloalkyl, C₁₋₆ alkoxy, —SO₂C₁₋₆alkyl and —COR⁷,
ii) a 5 to 10 membered heteroaromatic comprising 1, 2 or 3 heteroatoms selected from the group consisting of O, N and S optionally substituted by 1 or 2 substituents independently selected from the group consisting of halogen, cyano, C₁₋₆alkyl, C₁₋₆haloalkyl, C₁₋₆alkoxy and —COR⁷, or
iii) C₁₋₆alkyl, C₀₋₆alkylcyano, C₀₋₆alkylC₁₋₆alkoxy, C₀₋₂alkylC(O)R⁷ or cyclohexyl;

R² is C₁₋₆alkyl;

R³ is C₁₋₆alkyl;

R⁴ is:
i) H, halogen, cyano, C₁₋₆alkyl, C₁₋₆haloalkyl, C₁₋₆alkoxy, C₀₋₆hydroxyalkyl, —SO₂C₁₋₆alkyl, —C(O)NR⁸R⁹, —C(O)R¹°, —C₀₋₆alkyl-NR¹¹R¹², or
ii) —OₘC₁₋₆alkyl substituted by a 5 or 6 membered heterocyclyl or heteroaromatic each comprising 1, 2, 3 or 4 heteroatoms independently selected from the group consisting of N, O and S and wherein said hetercyclyl or heteroaromatic is optionally substituted by 1, 2 or 3 groups independently selected from the group consisting of halogen, cyano, C₁₋₆alkyl, C₁₋₆haloalkyl and C₁₋₆alkoxy, wherein m is 0, 1 or 2, wherein when the heterocyclyl or heteroatomic is linked through a heteroatom and m is 1, then the heteroatom and O are not directly linked if the resultant arrangement would be unstable;

R^{4a} is H, halogen, C₁₋₆alkyl, C₁₋₆haloalkyl, C₁₋₆alkoxy or C₀₋₆hydroxyalkyl;

R⁵ is H, halogen, C₁₋₆alkyl or C₁₋₆alkoxy;

R⁶ is H, C₁₋₆alkyl, C₀₋₆alkylcyano, C₀₋₆alkylC₁₋₆alkoxy or C₀₋₂alkylC(O)R⁷;

R⁷ is hydroxyl, C₁₋₆alkoxy, —NH₂, —NHC₁₋₆alkyl or N(C₁₋₆alkyl)₂; R⁸ and R⁹ independently are:
i) H, C₁₋₆alkyl, C₀₋₆alkylphenyl, C₀₋₆alkylheteroaromatic, C₃₋₆cycloalkyl, or
ii) R⁸ and R⁹ together with the N to which they are attached form a 5 or 6 membered heterocyclyl or heteroaromatic wherein said heterocyclyl or heteroaromatic may comprise 1, 2 or 3 further heteroatoms independently selected from the group consisting of O, N and S;

R¹⁰ is hydroxyl, C₁₋₆alkoxy or a 5 or 6 membered heterocyclyl or heteroaromatic comprising 1, 2, 3 or 4 heteroatoms selected from the group consisting of O, N and S;

R¹¹ and R¹² independently are:
i) H, C₁₋₆alkyl; or
ii) R¹¹ and R¹² together with the N to which they are attached form a 5 or 6 membered heterocyclyl or heteroaromatic wherein said heterocyclyl or heteroaromatic may comprise 1, 2 or 3 further heteroatoms independently selected from the group consisting of O, N and S.

In certain embodiments, compounds of Formula B or Formula C may be selected from the group consisting of:

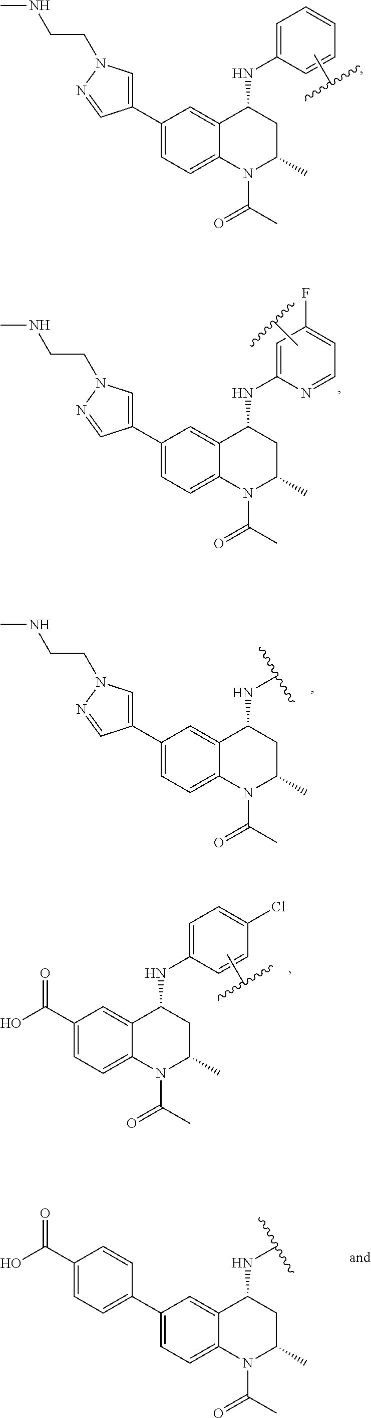

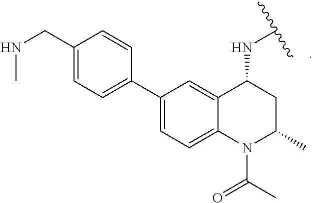 [SMILES: *N[C@@H]1C[C@H](C)N(C(C)=O)c2ccc(-c3ccc(CNC)cc3)cc21]

In another embodiment, exemplary bromodomain ligands include tetrahydroquinolines represented by the structures:

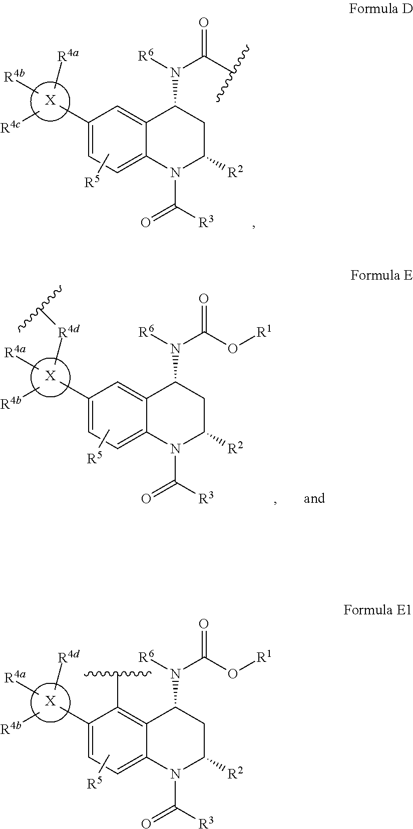

wherein:

R¹ is C₁₋₆alkyl, C₃₋₇cycloalkyl or benzyl;

R² is C₁₋₄alkyl;

R³ is C₁₋₄alkyl;

X is phenyl, naphthyl, or heteroaryl;

R^{4a} is hydrogen, C₁₋₄alkyl or is a group L-Y in which L is a single bond or a C₁₋₆alkylene group and Y is OH, OMe, CO₂H, CO₂C₁₋₆alkyl, CN, or NR⁷R⁸;

R⁷ and R⁸ are independently hydrogen, a heterocyclyl ring, C₁₋₆alkyl optionally substituted by hydroxyl, or a heterocyclyl ring; or

R⁷ and R⁸ combine together to form a heterocyclyl ring optionally substituted by C₁₋₆alkyl, CO₂C₁₋₆alkyl, NH₂, or oxo;

R^{4b} and R^{4c} are independently hydrogen, halogen, C₁₋₆alkyl, or C₁₋₆alkoxy;

R^{4d} is C₁₋₄alkyl or is a group -L-Y— in which L is a single bond or a C₁₋₆alkylene group and Y is —O—, —OCH₂—, —CO₂—, —CO₂C₁₋₆alkyl-, or —N(R⁷)—;

R⁵ is hydrogen, halogen, C₁₋₆alkyl, or C₁₋₆alkoxy;

R⁶ is hydrogen or C₁₋₄alkyl.

In some cases, compounds of Formula D or Formula E may be selected from the group consisting of:

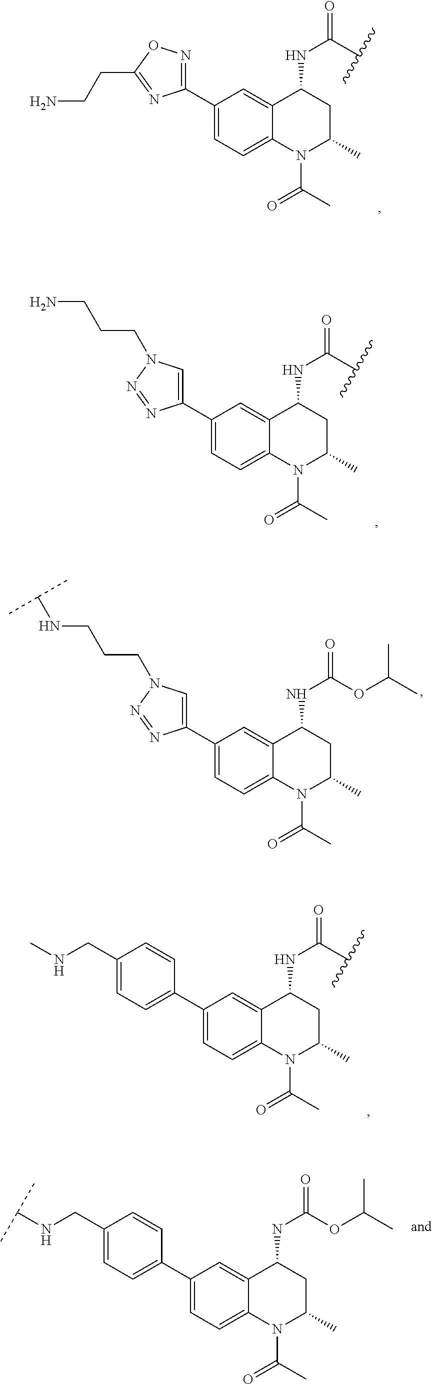

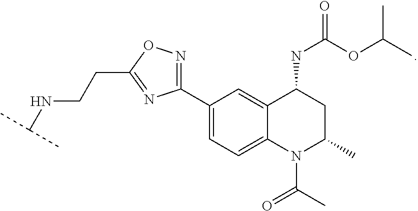

For example, compounds of Formula A, Formula B, Formula C, Formula D or Formula E may be selected from the group consisting of:

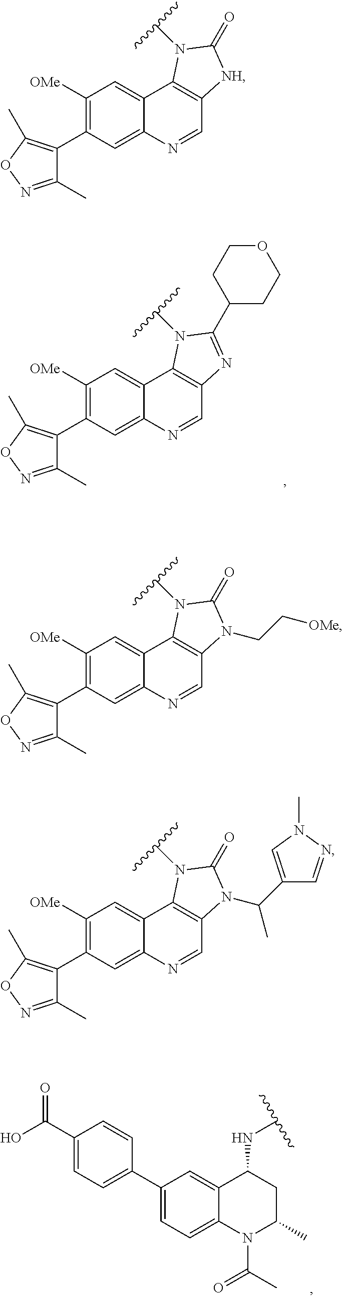 [SMILES: *n1c(=O)[nH]c2cnc3cc(-c4c(C)noc4C)c(OC)cc3c21 ; *n1c(C2CCOCC2)nc2cnc3cc(-c4c(C)noc4C)c(OC)cc3c21 ; *n1c(=O)n(CCOC)c2cnc3cc(-c4c(C)noc4C)c(OC)cc3c21 ; *n1c(=O)n(C(C)c2cnn(C)c2)c2cnc3cc(-c4c(C)noc4C)c(OC)cc3c21 ; *N[C@@H]1C[C@H](C)N(C(C)=O)c2ccc(-c3ccc(C(=O)O)cc3)cc21]

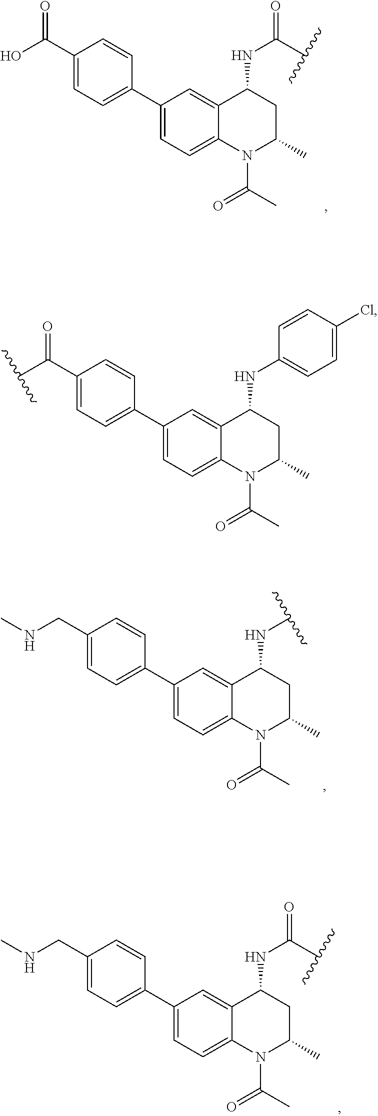

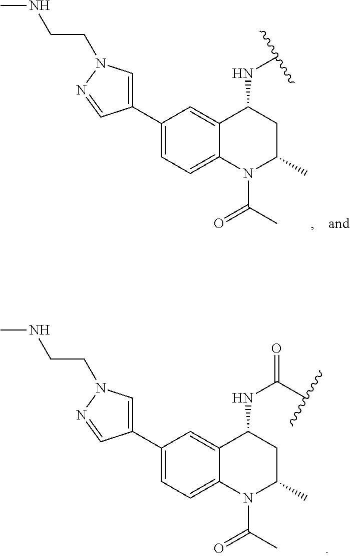

In another embodiment, exemplary bromodomain ligands are represented by the structures:

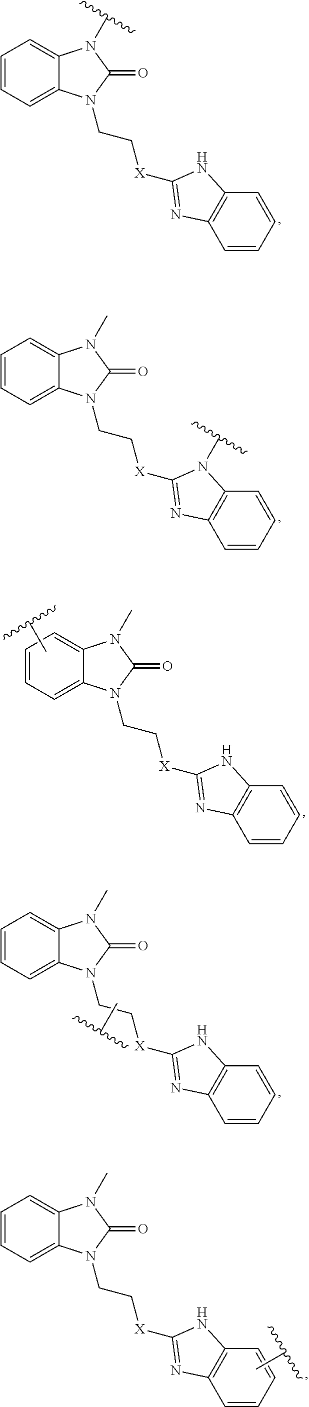

where X is O, NR⁴, or S, and R⁴ is independently selected from the group consisting of hydrogen, hydroxyl, halo, amino, thiol, C₁₋₆alkyl, haloC₁₋₆alkyl, C₁₋₆alkoxy, —NH—C₁₋₆alkyl, —S—C₁₋₆alkyl, haloC₁₋₆alkoxy, nitro, cyano, —CF₃, —OCF₃, —C(O)O—C₁₋₆alkyl, C₁₋₄alkylamino, phenoxy, benzoxy, and C₁₋₄alkylOH;

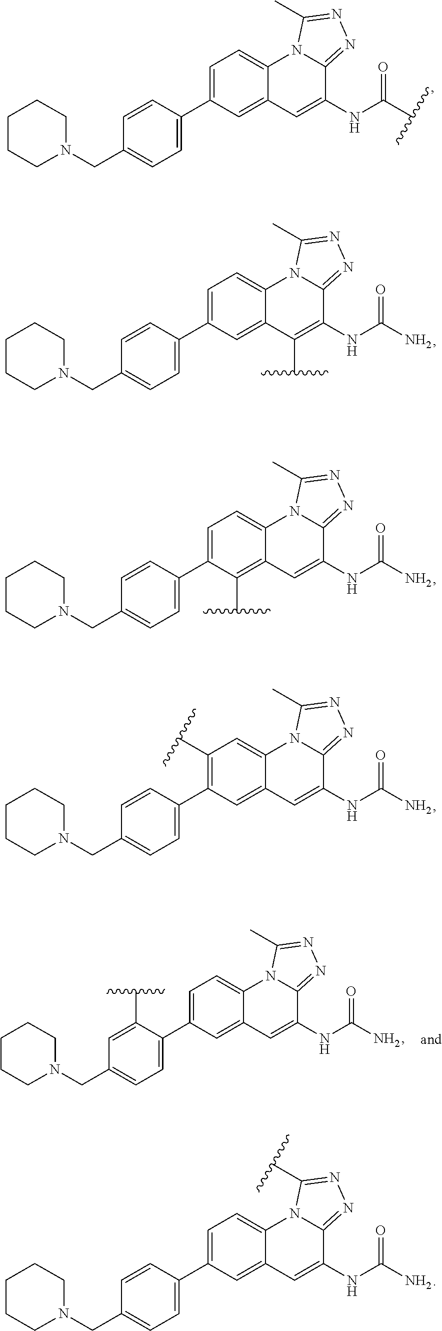 [SMILES: *C(=O)Nc1cc2cc(-c3ccc(CN4CCCCC4)cc3)ccc2n2c(C)nnc12 ; *c1c(NC(N)=O)c2nnc(C)n2c2ccc(-c3ccc(CN4CCCCC4)cc3)cc12 ; *c1c(-c2ccc(CN3CCCCC3)cc2)ccc2c1cc(NC(N)=O)c1nnc(C)n12 ; *c1cc2c(cc1-c1ccc(CN3CCCCC3)cc1)cc(NC(N)=O)c1nnc(C)n12 ; *c1cc(CN2CCCCC2)ccc1-c1ccc2c(c1)cc(NC(N)=O)c1nnc(C)n12 ; *c1nnc2c(NC(N)=O)cc3cc(-c4ccc(CN5CCCCC5)cc4)ccc3n12]

In another embodiment, exemplary bromodomain ligands include heterocycles represented by the structures:

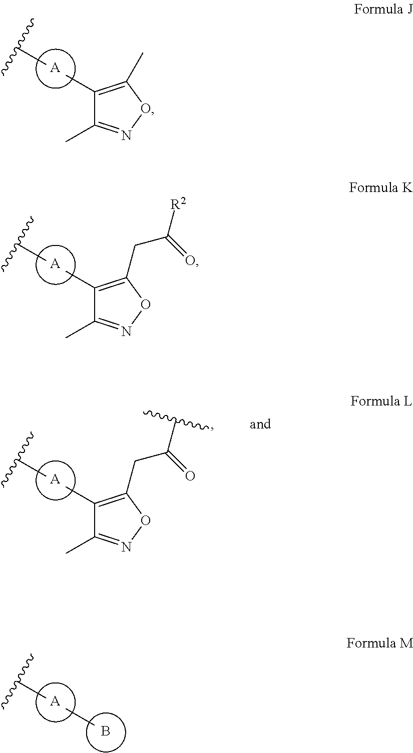

wherein:

A is independently, for each occurrence, a 4-8 membered cycloalkyl, heterocyclic, phenyl, naphthyl, or heteroaryl moiety, each optionally substituted with one, two, three or more R¹ substituents;

R¹ is selected from the group consisting of hydroxy, halogen, oxo, amino, imino, thiol, sulfanylidene, C₁₋₆alkyl, hydroxyC₁₋₆ alkyl, —O—C₁₋₆ alkyl, —NH—C₁₋₆ alkyl, —CO₂H—C(O)C₁₋₆alkyl, —C(O)O—C₁₋₆ alkyl, aminoC₁₋₆alkyl, haloC₁₋₆alkyl, —C₁₋₆alkylC(O)R², —O—C(O)R², —NH—C(O)R², —O—C₁₋₆alkyl-C(O)R², —NHC₁₋₆alkyl-C(O)R², acylaminoC₁₋₆alkyl, nitro, cyano, CF₃, —OCF₃, —OS(O)₂C₁₋₆alkyl, phenyl, naphthyl, phenyloxy, —NH-phenyl, benzyloxy, and phenylmethoxy halogen; wherein C₁₋₆alkyl, phenyl, and naphthyl are optionally substituted by one two or three substituents selected from the group consisting of hydroxyl, halogen, amino, nitro, phenyl and C₁₋₆alkyl; or two R¹ substitutents may be taken together with the atoms to which they are attached to form a fused aliphatic or heterocyclic bicyclic ring system;

R² is —NR^{2a}R^{2a′} or —OR^{2b}; wherein one of R^{2a} or R^{2a′} is hydrogen, and R^{2b} or the other of R^{2a} or R^{2a′} is selected from the group consisting of C₁₋₆alkyl, haloC₁₋₆alkyl, R^{2c}R^{2c′}N—C₂₋₆alkyl, carbocyclyl, carbocyclyloC₁₋₄alkyl, heterocyclyl and heterocyclylC₁₋₄alkyl, wherein any of the carbocyclyl or heterocyclyl groups are optionally substituted by one or more substituents selected from the group consisting of halogen, C₁₋₆alkyl, haloC₁₋₆alkyl, C₁₋₆alkoxy, haloC₁₋₆alkoxy, carbonyl, —CO-carbocyclyl, azido, amino, hydroxyl, nitro and cyano, wherein the —CO-carbocyclyl group may be optionally substituted by one or more substituents selected from the group consisting of halogen, C₁₋₆alkyl, haloC₁₋₆alkyl, C₁₋₆alkoxy, haloC₁₋₆alkoxy, azido, nitro and cyano; or

two adjacent groups on any of the carbocyclyl or heterocyclyl groups together with the interconnecting atoms form a 5- or 6-membered ring which ring may contain 1 or 2 heteroatoms independently selected from the group consisting of O, S and N; or R^{2a} and R^{2a′} together with the N atom to which they are attached form a 4-, 5-, 6- or 7-membered ring which optionally contains 1 or 2 heteroatoms independently selected from the group consisting of O, S and N; wherein the 4-, 5-, 6 or 7-membered ring is optionally substituted by C₁₋₆alkyl, hydroxyl or amino;

R^{2c} and R^{2c′} are independently hydrogen or C₁₋₆alkyl;

B is selected from the group consisting of:

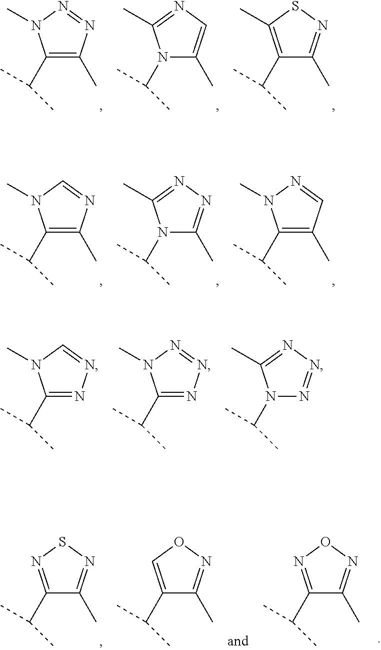

In one embodiment, compounds of Formula J may be selected from the group consisting of:

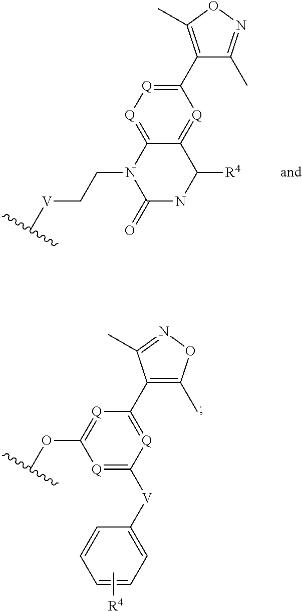

wherein:

Q is independently, for each occurrence, N or CH;

V is independently, for each occurrence, O, S, NH, or a bond; and

R⁴ is independently selected from the group consisting of hydrogen, hydroxyl, halo, amino, thiol, C₁₋₆ alkyl, halo C₁₋₆ alkyl, C₁₋₆ alkoxy, —NH—C₁₋₆ alkyl, —S—C₁₋₆ alkyl, haloC₁₋₆ alkoxy, nitro, cyano, —CF₃, —OCF₃, —C₁₋₄alkylamino, phenoxy, benzoxy, and C₁₋4alkylOH.

For example, compounds of Formula J or Formula L may be selected from the group consisting of:

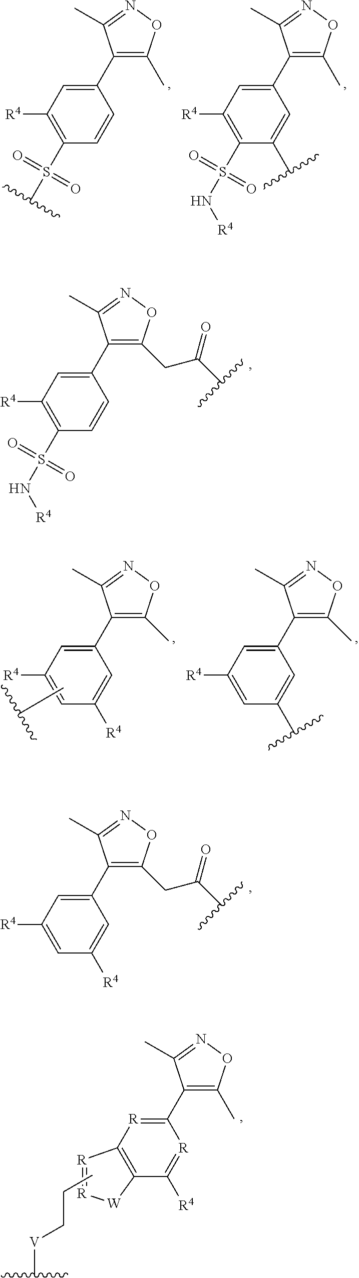

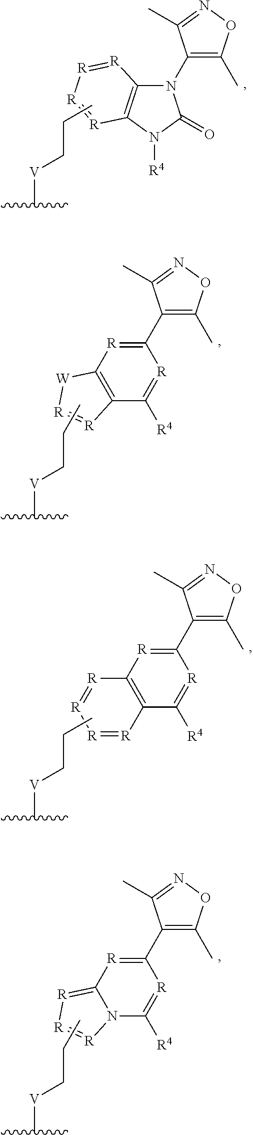

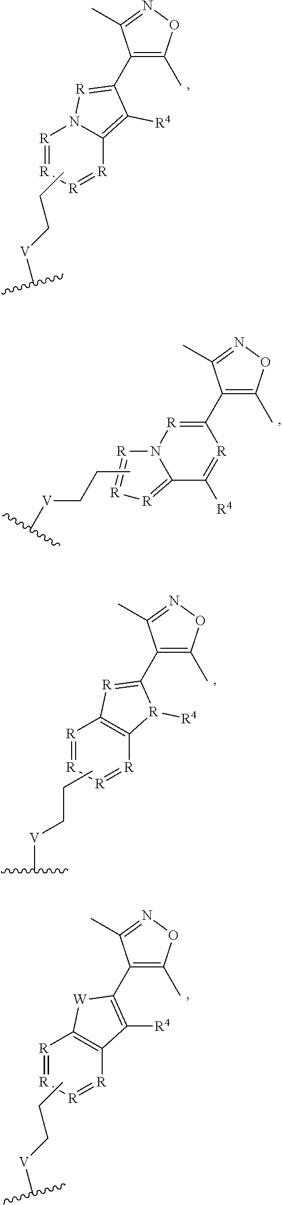

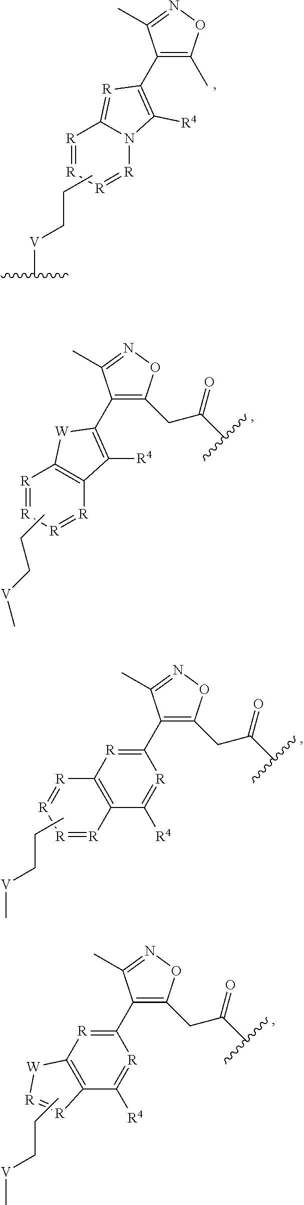

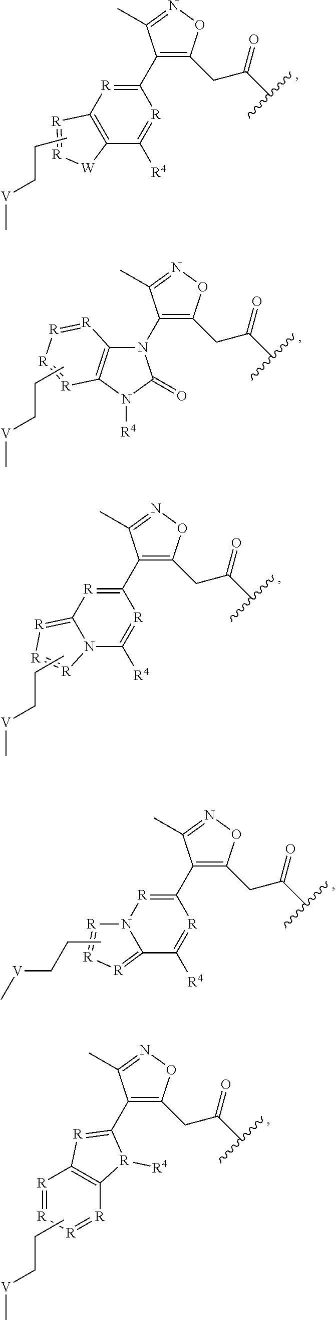

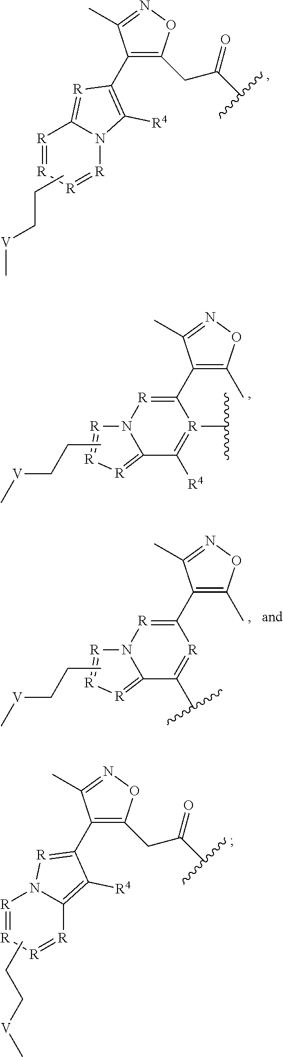

wherein:

R is independently, for each occurrence, N or CH;

V is independently, for each occurrence, a bond, O or NR⁴;

R⁴ is independently, for each occurrence, hydrogen, hydroxyl, halo, amino, —SO₂, thiol, C₁₋₆ alkyl, haloC₁₋₆ alkyl, C₁₋₆ alkoxy, —NH—C₁₋₆ alkyl, —S—C₁₋₆ alkyl, haloC₁₋₆ alkoxy, nitro, cyano, —CF₃, —OCF₃, —C(O)O—C₁₋₆alkyl, —C₁₋₆alkylamino, phenoxy, benzoxy, phenyl, naphthyl, heteroaryl and C₁₋₄alkylOH; wherein C₁₋₆alkyl, phenyl, and naphthyl are optionally substituted with 1, 2, 3 or more substituents selected from the group consisting of halogen, hydroxyl, amino and C₁₋₆alkyl; and

W is independently, for each occurrence,

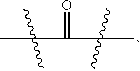 [SMILES: *C(*)=O]

### O, S, or NR⁴.

In another embodiment, compounds of Formula M may be selected from the group consisting of:

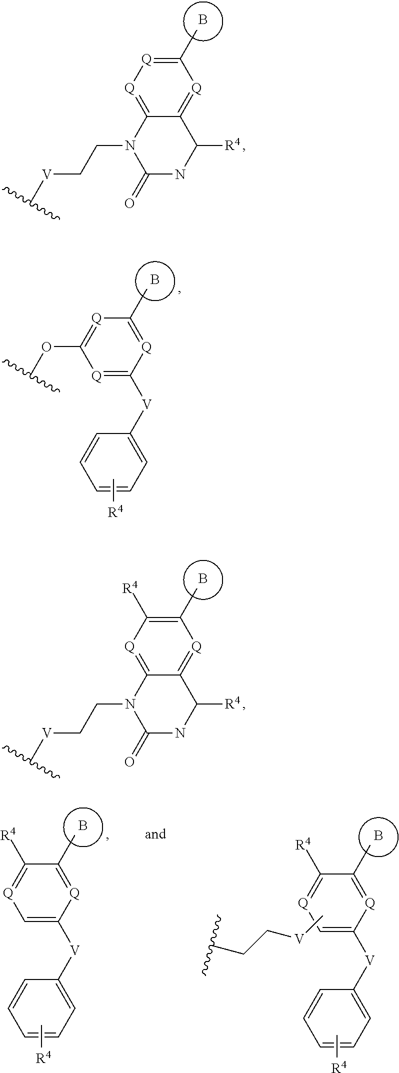

wherein:

B is selected from the group consisting of:

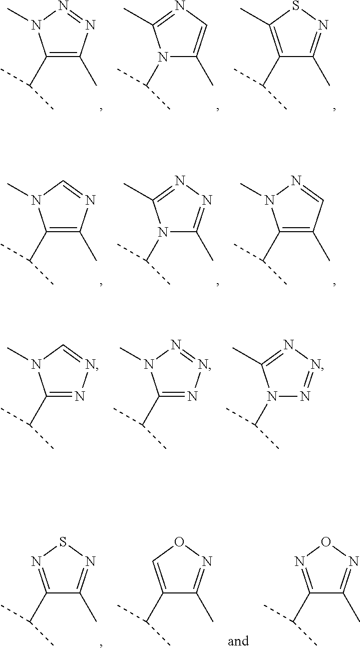

Q is independently, for each occurrence, N or CH;

V is independently, for each occurrence, O, S, NR⁴, or a bond; and

R⁴ is independently selected from the group consisting of hydrogen, hydroxyl, halo, amino, thiol, C₁₋₆ alkyl, haloC₁₋₆ alkyl, C₁₋₆ alkoxy, —NH—C₁₋₆ alkyl, —S—C₁₋₆ alkyl, haloC₁₋₆ alkoxy, nitro, cyano, —CF₃, —OCF₃, —C(O)O—C₁₋₆alkyl, —C₁₋₄alkylamino, phenoxy, benzoxy, and C₁₋4alkylOH.

For example, compounds of Formula J, Formula K, Formula L or Formula M may be selected from the group consisting of:

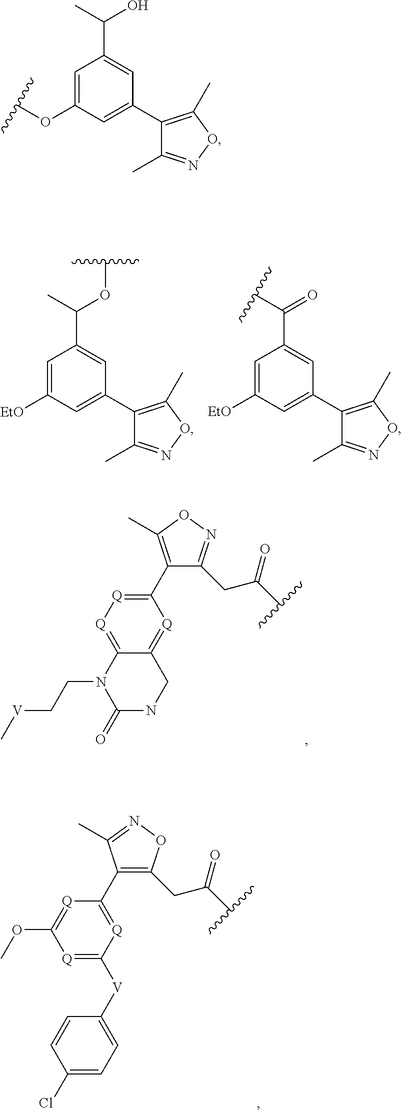

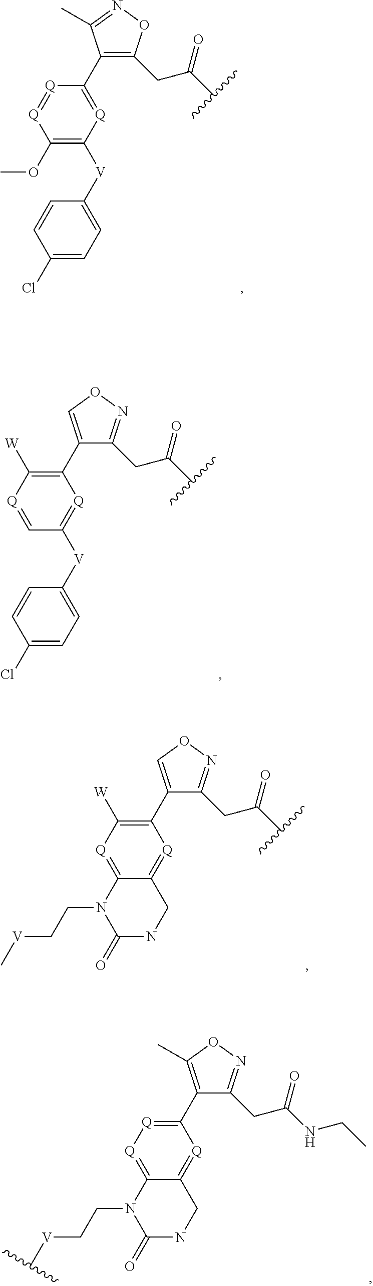

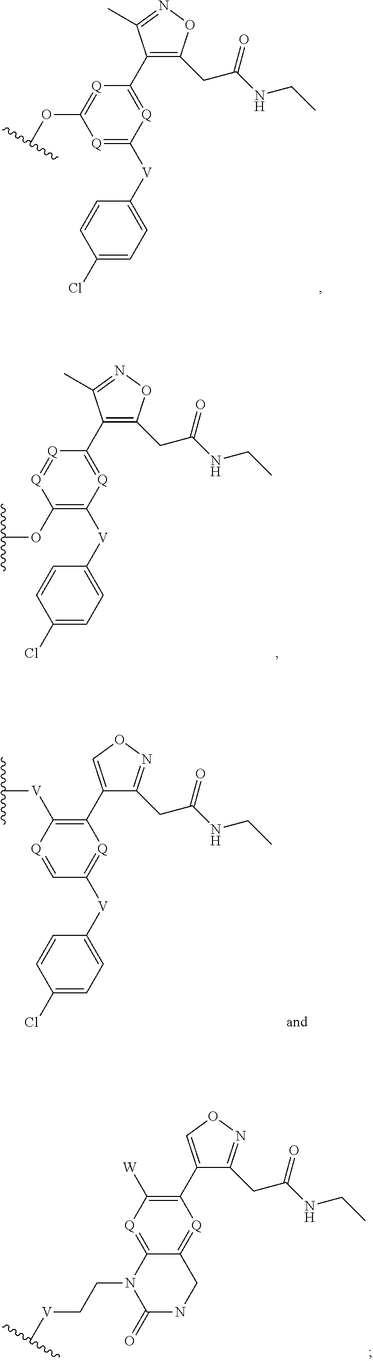

wherein:

Q is independently, for each occurrence, N or CH;

V is independently, for each occurrence, O, S, NR⁴, or a bond;

W is independently, for each occurrence, H, halogen, C₁₋₆alkyl, C₁₋₆alkoxy, —NH—C₁₋6alkyl, or —S—C₁₋₆alkyl; and

R⁴ is independently selected from the group consisting of hydrogen, hydroxyl, halo, amino, thiol, C₁₋₆ alkyl, halo C₁₋₆ alkyl, C₁₋₆ alkoxy, —NH—C₁₋₆ alkyl, —S—C₁₋₆ alkyl, haloC₁₋₆ alkoxy, nitro, cyano, —CF₃, —OCF₃, —C(O)O—C₁₋₆alkyl, —C₁₋₄alkylamino, phenoxy, benzoxy, and C₁₋4alkylOH.

In another embodiment, exemplary bromodomain ligands include compounds represented by the structures:

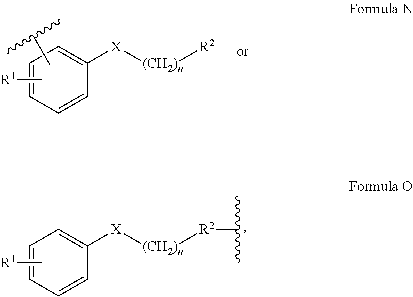

wherein:

R¹ is selected from the group consisting of hydrogen, lower alkyl, phenyl, naphthyl, aralkyl, heteroalkyl, SO₂, NH₂, NO₂, CH₃, CH₂CH₃, OCH₃, OCOCH₃, CH₂COCH₃, OH, CN, and halogen;

R² is selected from the group consisting of hydrogen, lower alkyl, aralkyl, heteroalkyl, phenyl, naphthyl, SO₂, NH₂, NH₃⁺, NO₂, CH₃, CH₂CH₃, OCH₃, OCOCH₃, CH₂COCH₃, OH, halogen, carboxy, and alkoxy;

X is selected from the group consisting of lower alkyl, SO₂, NH, NO₂, CH₃, CH₂CH₃, OCH₃, OCOCH₃, CH₂COCH₃, OH, carboxy, and alkoxy; and

n is an integer from 0 to 10.

For example, compounds of Formula N or Formula O may be selected from the group consisting of:

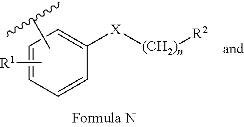

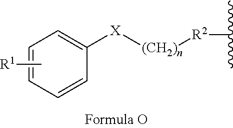

| R¹ | X | n | R² |
|---|---|---|---|
| 2-NO₂ | NH | 3 | —NH₃⁺ |
| 2-NO₂, 4-CH₃ | NH | 3 | —NH₃⁺ |
| 2-NO₂, 4-CH₂—CH₃ | NH | 3 | —NH₃⁺ |
| 2-NO₂, 3-CH₃ | NH | 3 | —NH₃⁺ |
| 2-NO₂, 5-CH₃ | NH | 3 | —NH₃⁺ |
| 2-NO₂, 4-Ph | NH | 3 | —NH₃⁺ |
| 2-NO₂, 4-CN | NH | 3 | —NH₃⁺ |
| 2-NO₂, 5-CN | NH | 3 | —NH₃⁺ |
| 2-CH₃, 5-NO₂ | NH | 3 | —NH₃⁺ |
| 2-COO⁻ | NH | 3 | —NH₃⁺ |
| 2-COOCH₃ | NH | 3 | —NH₃⁺ |
| 2-NO₂ | O | 3 | —NH₃⁺ |
| 2-NO₂, 4-CH₃ | O | 3 | —NH₃⁺ |
| 2-NO₂, 4-CH₃O | O | 3 | —NH₃⁺ |
| 2-NO₂, 4-Cl | O | 3 | —NH₃⁺ |
| 2-NO₂, 5-CH₃ | O | 3 | —NH₃⁺ |
| 2-NO₂, 3-CH₃ | O | 3 | —NH₃⁺ |
| 2-NO₂ | CH₂ | 3 | —NH₃⁺ |
| 2-NO₂ | NH | 4 | —NH₃⁺ |
| 4-NO₂ | NH | 2 | —NH₃⁺ |
| 4-NO₂ | NH | 4 | —NH₃⁺ |
| 3-NH₂, 4-NO₂ | NH | 3 | —COO⁻ |
| 2-NO₂, 4-Cl | NH | 2 | —(OH)CH₃ |
| 2-Cl, 4-NO₂ | NH | 2 | —(OH)CH₃ |

For example, the compound may be

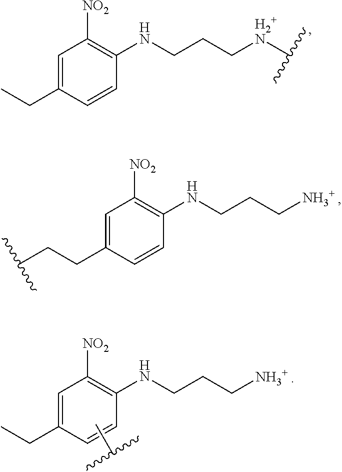

In some embodiments, a ligand may be selected from the group consisting of:

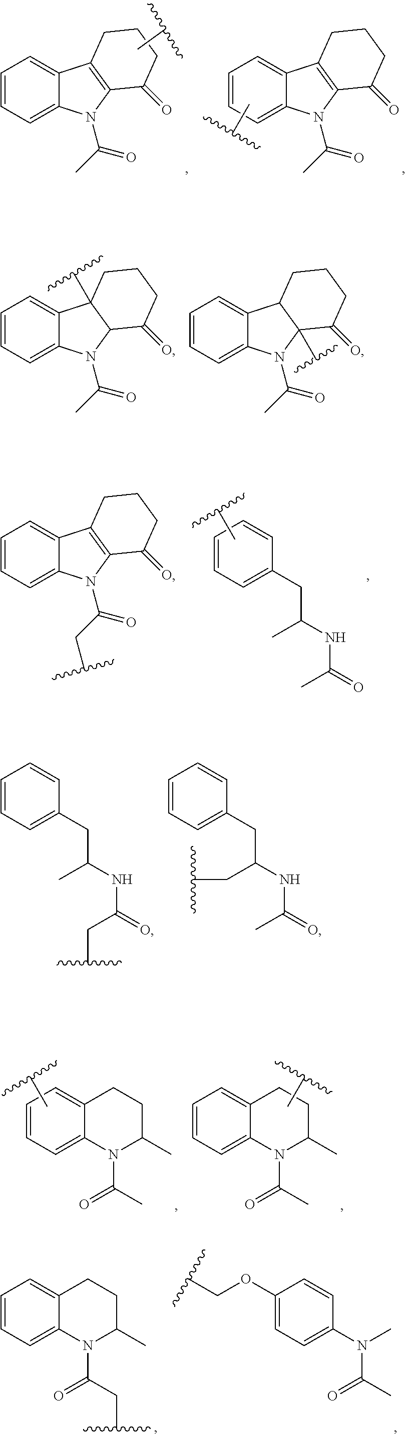

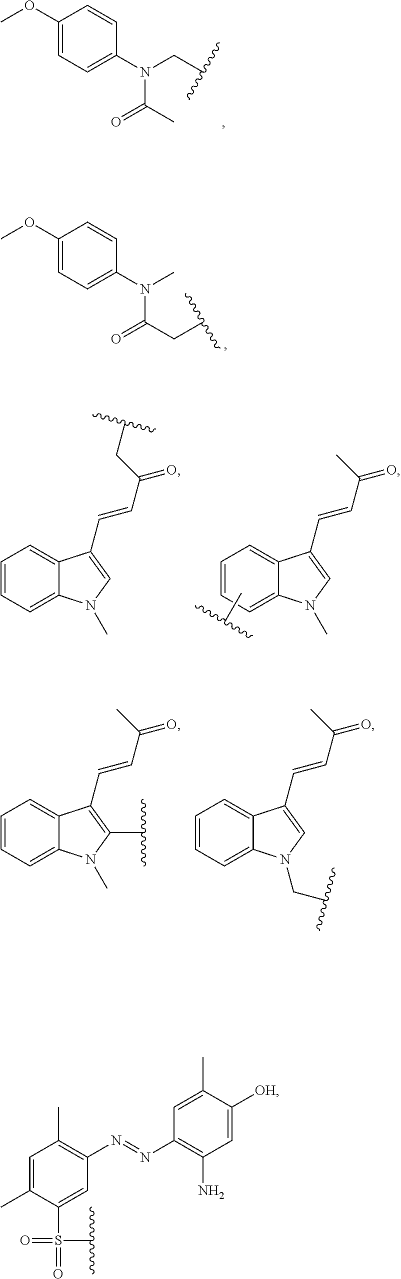

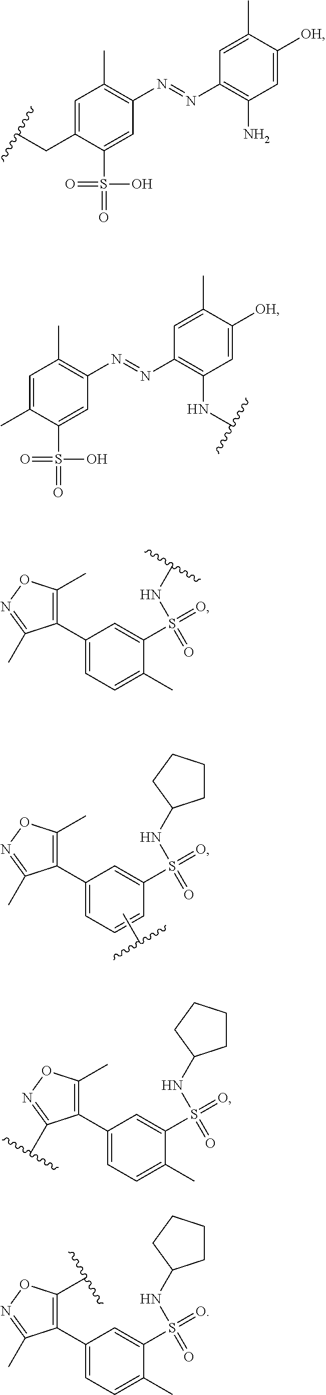

In yet another embodiment, exemplary bromodomain ligands include compounds represented by the structures:

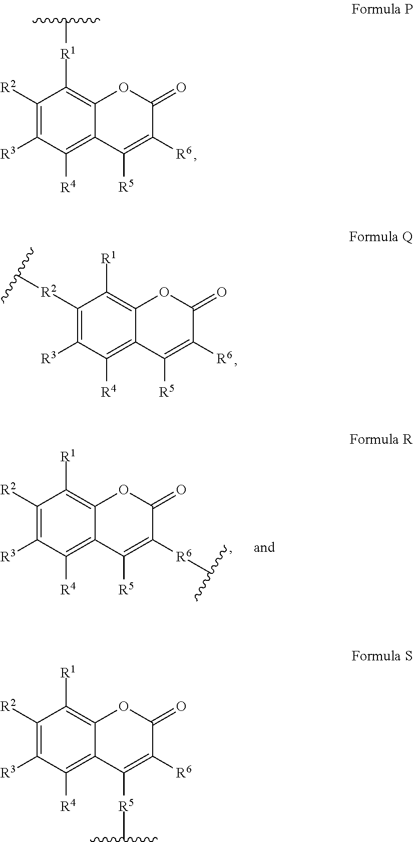

wherein:

R¹, R², R³, R⁴, R⁵, and R⁶ are independently selected from the group consisting of hydrogen, lower alkyl, phenyl, naphthyl, aralkyl, heteroaryl, SO₂, NH₂, NH₃⁺, NO², SO², CH³, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂OH, NH(CH₂)₃CH₃, NHCH₃, SH, halogen, carboxy, and alkoxy.

In some embodiments, compounds of Formula P, Formula Q, Formula R, or Formula S may be selected from the group consisting of:

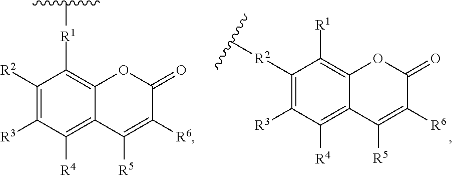

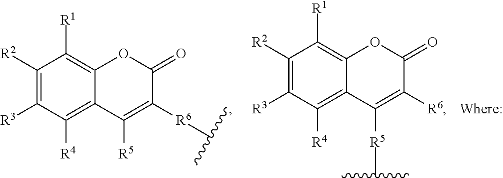

| R¹ | R² | R³ | R⁴ | R⁵ | R⁶ |
|---|---|---|---|---|---|
| —OH | —OH | H | H | —CH₃ | H |
| —OH | —OH | H | H | —CH₂—CH₂—CH₃ | H |
| —OH | —OH | H | H | —(CH₂)₂—CH₃ | H |
| —OH | —OH | H | H | —Ph | H |
| —OH | —OH | H | H | -cyclopentane | H |
| H | —OH | H | H | —CH₃ | H |
| H | —OH | H | H | —CH₂—CH₂—CH₃ | H |
| H | —OH | H | —OH | —CH₂—CH₂—CH₃ | H |
| H | —CH₃ | H | —OH | —CH₂—CH₂—CH₃ | H |
| H | —CH₃ | H | —OH | —CH₂—CH₃ | H |
| H | —O—CH₃ | H | H | —CH₂—CH₃ | H |
| H | —O—CH₃ | H | H | —CH₂—CH₂—CH₃ | H |
| H | —O—CH₂—CH₃ | H | H | —CH₂—CH₃ | H |
| H | —O—CH₂—CH₃ | H | H | —CH₂—CH₂—CH₃ | H |
| H | —O—CH(CH₃)₂ | H | H | —CH₃ | H |
| H | —O—CO—CH₃ | H | H | —CH₂—CH₂—CH₃ | H |
| H | —O—CH₂—CO—OH | H | H | —CH₂—CH₃ | H |
| H | —O—CH₂—CO—OH | H | H | —(CH₂)₂—CH₃ | H |
| H | —O—CH(CH₃)—CO—OH | H | H | —(CH₂)₂—CH₃ | H |
| H | —O—CH₂—CO—CH₃ | H | H | —CH₂—CH₃ | H |
| H | —O—CH₂—CO—NH₂ | H | H | —CH₂—CH₃ | H |
| H | —O—CO—CH₃ | H | H | —CH₃ | H |
| H | —O—CO—CH(CH₃)₂ | H | H | —CH₃ | H |
| H | —O—CH₂—CO—CH₃ | H | H | —CH₃ | H |
| H | —O—CH₂—CH₂—OH | H | H | —CH₃ | H |
| H | —O—CH₂—CH₂—CH₃ | H | H | —CH₃ | H |
| H | —O—CH₂—CH₂—CH₃ | H | H | H | H |
| H | —O—(CH₂)₃—CH₃ | H | H | H | H |
| H | —O—CH(CH₃)—CO—OCH₃ | H | H | H | H |
| H | —O—CH₂—CO—N(CH₃)₂ | H | H | H | H |
| H | —O—CH(CH₃)₂ | H | H | —cyclophentane | H |
| H | —O—CH₂—CH₃ | —O—CH₂—CH₃ | H | —CH₃ | H |
| H | —CH₃ | —NH—CO—CH₃ | H | —CH₃ | H |
| H | H | H | H | —NH—(CH₂)₃—N(CH₃)₂ | —NH₂ |
| H | H | H | H | —NH—(CH₂)₂—N(CH₃)₂ | —NH₂ |
| H | H | H | H | —NH—(CH₂)₂—OH | —NH₂ |
| H | H | H | H | —NH—(CH₂)₃—CH₃ | —NH₂ |
| H | H | H | H | —NH—CH₃ | —NH₂ |
| H | H | H | H | —NH₂ | —NH₂ |

For example, the compound may be selected from the group consisting of:

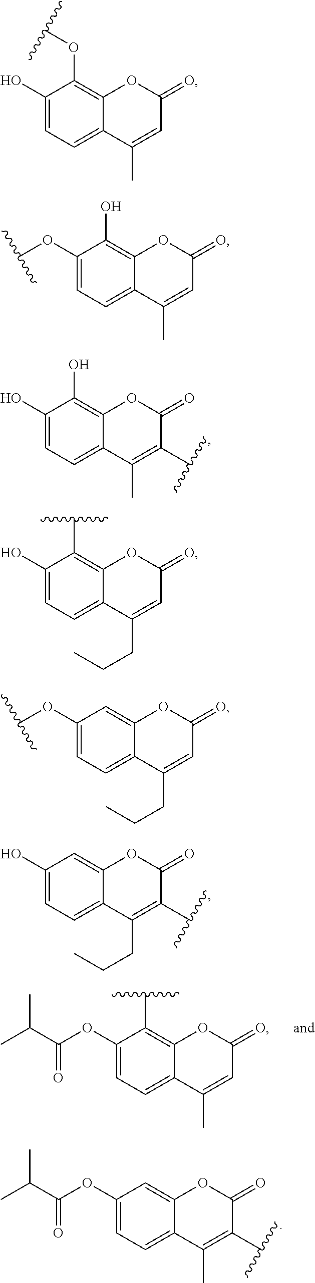

In still another embodiment, exemplary bromodomain ligands include compounds represented by the structure:

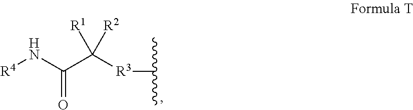

wherein:

R¹, R², and R³ are independently selected from the group consisting of hydrogen, lower alkyl, phenyl, naphthyl, aralkyl, heteroaryl, SO₂, NH₂, NH₃⁺, NO₂, SO₂, CH₃, CH₂CH₃, OCH₃, OCOCH₃, CH₂COCH₃, OH, SH, halogen, carboxy, and alkoxy; R⁴ is selected from the group consisting of lower alkyl, phenyl, naphthyl, SO₂, NH, NO₂, CH₃, CH₂CH₃, OCH₃, OCOCH₃, CH₂COCH₃, OH, carboxy, and alkoxy.

In yet another embodiment, exemplary bromodomain ligands include compounds represented by the structures:

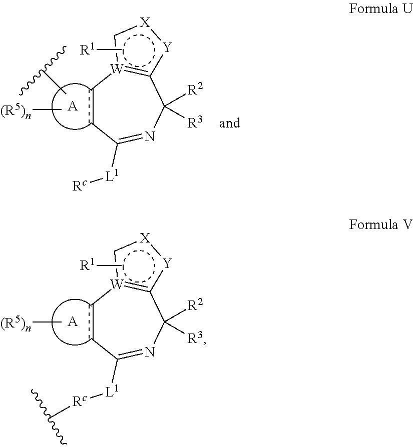

or a pharmaceutically acceptable salt thereof,

wherein:

X is O or N;

Y is O or N; wherein at least one of X or Y is O;

W is C or N;

R¹ is H, alkyl, alkenyl, alkynyl, aralkyl, phenyl, naphthyl, heteroaryl, halo, CN, OR^{A}, NR^{A}R^{B}, N(R^{A})S(O)_{q}R^{A}R^{B}, N(R^{A})C(O)R^{B}, N(R^{A})C(O)NR^{A}R^{B}, N(R^{A})C(O)OR^{A}, N(R^{A})C(S)NR^{A}R^{B}, S(O)_{q}R^{A}, C(O)R^{A}, C(O)OR^{A}, OC(O)R^{A}, or C(O)NR^{A}R^{B};

each R^{A} is independently alkyl, alkenyl, or alkynyl, each containing 0, 1, 2, or 3 heteroatoms selected from O, S, or N; phenyl; naphthyl, heteroaryl; heterocyclic; carbocyclic; or hydrogen;

each R^{B} is independently alkyl, alkenyl, or alkynyl, each containing 0, 1, 2, or 3 heteroatoms selected from O, S, or N; phenyl; naphthyl; heteroaryl; heterocyclic; carbocyclic; or hydrogen; or

R^{A} and R^{B}, together with the atoms to which each is attached, can form a heterocycloalkyl or a heteroaryl; each of which is optionally substituted;

Ring A is cycloalkyl, phenyl, naphthyl, heterocycloalkyl, or heteroaryl;

R^{C} is alkyl, alkenyl, alkynyl, cycloalkyl, phenyl, naphthyl, heterocycloalkyl, or heteroaryl, each optionally substituted with 1-5 independently selected R⁴, and when L¹ is other than a covalent bond, R^{C} is additionally selected from H;

R² and R³ are each independently H, halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″), or —(CH₂)ₚR^{x}; or

R₂ and R₃ together with the atoms to which each is attached, form an optionally substituted 3-7 membered saturated or unsaturated spiro-fused ring having 0-3 heteroatoms independently selected from nitrogen, oxygen, or sulfur;

each R^{x} is independently halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″);

L¹ is a covalent bond or an optionally substituted bivalent C₁₋₆ hydrocarbon chain wherein one or two methylene units is optionally replaced by —NR′—, —N(R′)C(O)—, —C(O)N(R′)—, —N(R′)SO₂—, —SO₂N(R′)— —O—, —C(O)—, —OC(O)—, —C(O)O—, —S—, —SO— or —SO₂—;

each R is independently hydrogen, alkyl, alkenyl, alkynyl, phenyl, naphthyl, aralkyl, cycloalkyl, heteroaryl, or heterocycloalkyl;

each R′ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an heteroaryl or heterocycloalkyl group; each R″ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an heteroaryl or heterocycloalkyl group; or

R′ and R″, together with the atoms to which each is attached, can form cycloalkyl, heterocycloalkyl, phenyl, naphthyl, or heteroaryl; each of which is optionally substituted;

each R⁴ is independently alkyl, alkenyl, alkynyl, phenyl, naphthyl, aralkyl, cycloalkyl, heteroaryl, or heterocycloalkyl, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);

each R⁵ is independently —R, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);

n is 0-5;

each q is independently 0, 1, or 2; and

p is 1-6.

In still another embodiment, exemplary bromodomain ligands include compounds represented by the structure:

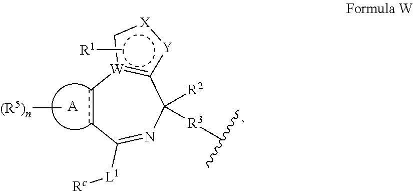

wherein:

X is O or N;

Y is O or N; wherein at least one of X or Y is O;

W is C or N;

R¹ is H, alkyl, alkenyl, alkynyl, aralkyl, phenyl, naphthyl, heteroaryl, halo, CN, OR^{A}, NR^{A}R^{B}, N(R^{A})S(O)_{q}R^{A}R^{B}, N(R^{A})C(O)R^{B}, N(R^{A})C(O)NR^{A}R^{B}, N(R^{A})C(O)OR^{A}, N(R^{A})C(S)NR^{A}R^{B}, S(O)_{q}R^{A}, C(O)R^{A}, C(O)OR^{A}, OC(O)R^{A}, or C(O)NR^{A}R^{B};

each R^{A} is independently optionally substituted alkyl, optionally substituted alkenyl or optionally substituted alkynyl, each containing 0, 1, 2, or 3 heteroatoms selected from O, S, or N; phenyl; naphthyl; heteroaryl; heterocyclic; carbocyclic; or hydrogen;

each R^{B} is independently alkyl, alkenyl, or alkynyl, each containing 0, 1, 2, or 3 heteroatoms selected from O, S, or N; phenyl; naphthyl; heteroaryl; heterocyclic; carbocyclic; or hydrogen; or

R^{A} and R^{B}, together with the atoms to which each is attached, can form a heterocycloalkyl or a heteroaryl; each of which is optionally substituted;

Ring A is cycloalkyl, phenyl, naphthyl, heterocycloalkyl, or heteroaryl;

R^{C} is alkyl, alkenyl, alkynyl, cycloalkyl, phenyl, naphthyl, heterocycloalkyl, or heteroaryl, each optionally substituted with 1-5 independently selected R⁴, and when L¹ is other than a covalent bond, R^{C} is additionally selected from H;

R² is H, halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″), or —(CH₂)ₚR^{x};

R³ is a bond or optionally substituted alkyl; or

R₂ and R₃ together with the atoms to which each is attached, form an optionally substituted 3-7 membered saturated or unsaturated spiro-fused ring having 0-3 heteroatoms independently selected from nitrogen, oxygen, or sulfur;

each R^{x} is independently halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″);

L¹ is a covalent bond or an optionally substituted bivalent C₁₋₆ hydrocarbon chain wherein one or two methylene units is optionally replaced by —NR′—, —N(R′)C(O)—, —C(O)N(R′)—, —N(R′)SO₂—, —SO₂N(R′)—, —O—, —C(O)—, —OC(O)—, —C(O)O—, —S—, —SO—, or —SO₂—;

each R is independently hydrogen, alkyl, alkenyl, alkynyl, phenyl, naphthyl, aralkyl, cycloalkyl, heteroaryl, or heterocycloalkyl;

each R′ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an heteroaryl or heterocycloalkyl group; each R″ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an optionally substituted heteroaryl or heterocycloalkyl group; or

R′ and R″, together with the atoms to which each is attached, can form cycloalkyl, heterocycloalkyl, phenyl, naphthyl, or heteroaryl; each of which is optionally substituted;

each R⁴ is independently alkyl, alkenyl, alkynyl, phenyl, naphthyl, aralkyl, cycloalkyl, heteroaryl, or heterocycloalkyl, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);

each R⁵ is independently —R, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);

n is 0-5;

each q is independently 0, 1, or 2; and

p is 1-6.

In yet another embodiment, compounds of Formula U, Formula V, and Formula W may be selected from the group consisting of:

Structure

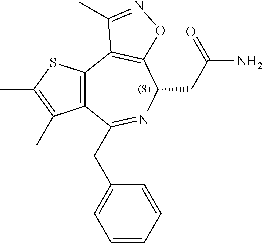

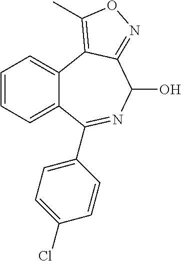 [SMILES: Cc1onc2c1-c1ccccc1C(c1ccc(Cl)cc1)=NC2O]

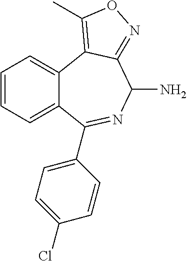 [SMILES: Cc1onc2c1-c1ccccc1C(c1ccc(Cl)cc1)=NC2N]

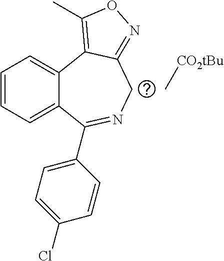

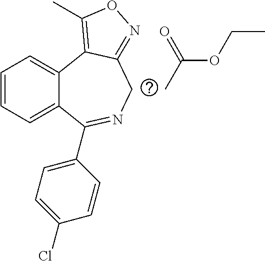

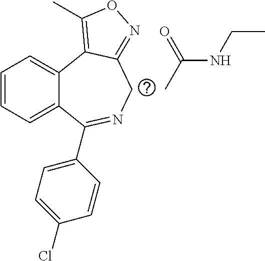

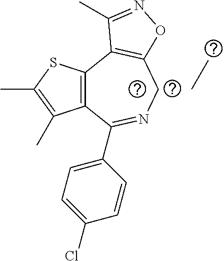

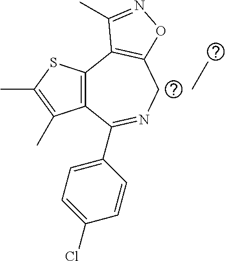

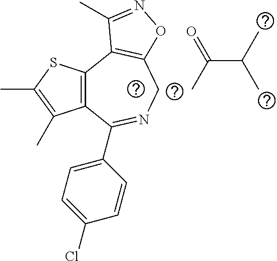

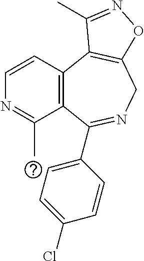 [SMILES: Cc1nccc2c1C(c1ccc(Cl)cc1)=NCc1onc(C)c1-2]

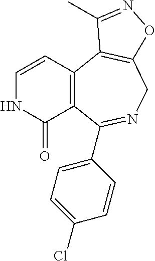 [SMILES: Cc1noc2c1-c1cc[nH]c(=O)c1C(c1ccc(Cl)cc1)=NC2]

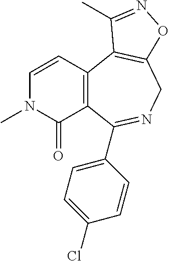 [SMILES: Cc1noc2c1-c1ccn(C)c(=O)c1C(c1ccc(Cl)cc1)=NC2]

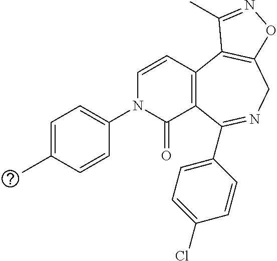 [SMILES: Cc1ccc(-n2ccc3c(c2=O)C(c2ccc(Cl)cc2)=NCc2onc(C)c2-3)cc1]

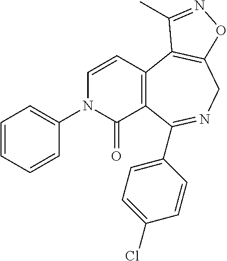 [SMILES: Cc1noc2c1-c1ccn(-c3ccccc3)c(=O)c1C(c1ccc(Cl)cc1)=NC2]

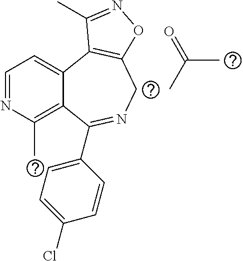

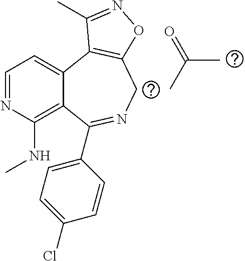

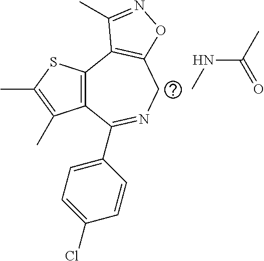

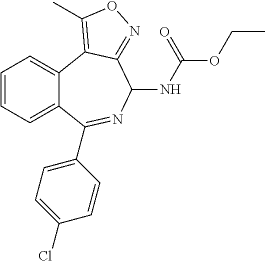 [SMILES: CCOC(=O)NC1N=C(c2ccc(Cl)cc2)c2ccccc2-c2c1noc2C]

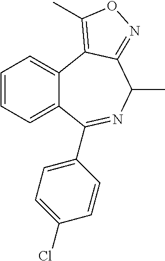 [SMILES: Cc1onc2c1-c1ccccc1C(c1ccc(Cl)cc1)=NC2C]

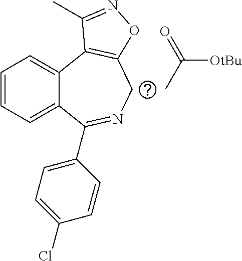

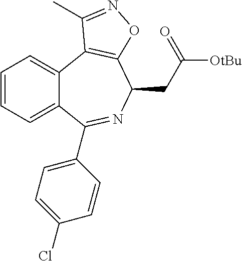 [SMILES: Cc1noc2c1-c1ccccc1C(c1ccc(Cl)cc1)=N[C@@H]2CC(=O)OC(C)(C)C]

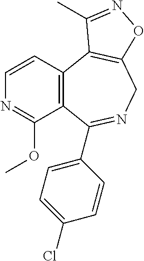 [SMILES: COc1nccc2c1C(c1ccc(Cl)cc1)=NCc1onc(C)c1-2]

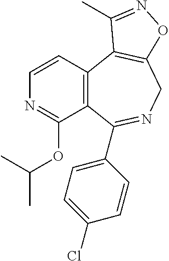 [SMILES: Cc1noc2c1-c1ccnc(OC(C)C)c1C(c1ccc(Cl)cc1)=NC2]

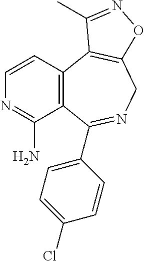 [SMILES: Cc1noc2c1-c1ccnc(N)c1C(c1ccc(Cl)cc1)=NC2]

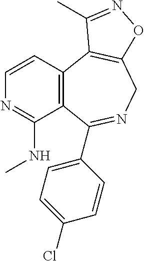 [SMILES: CNc1nccc2c1C(c1ccc(Cl)cc1)=NCc1onc(C)c1-2]

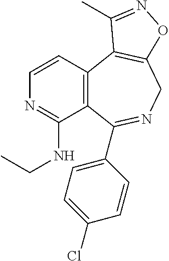 [SMILES: CCNc1nccc2c1C(c1ccc(Cl)cc1)=NCc1onc(C)c1-2]

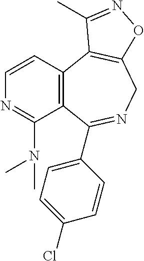 [SMILES: Cc1noc2c1-c1ccnc(N(C)C)c1C(c1ccc(Cl)cc1)=NC2]

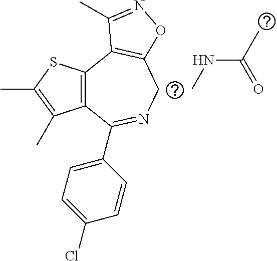

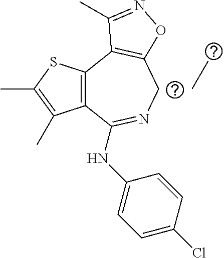

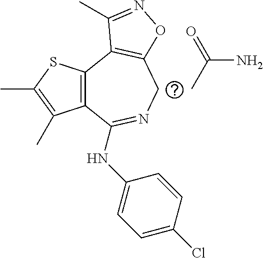

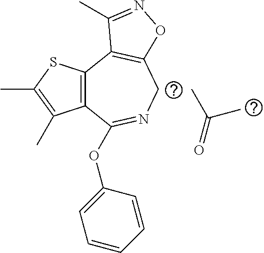

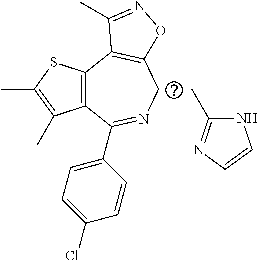

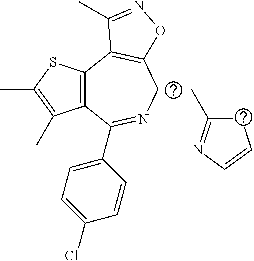

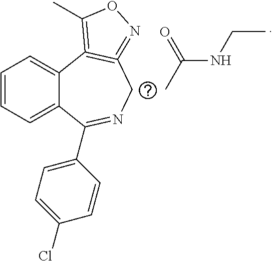

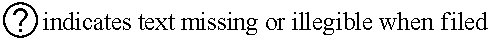

It will be appreciated that each of these compounds may be connected to a —Y—Z moiety, for example, as illustrated for generic structures Formula U, Formula V, and Formula W above.

For example, compounds of Formula U, Formula V, and Formula W may be selected from the group consisting of:

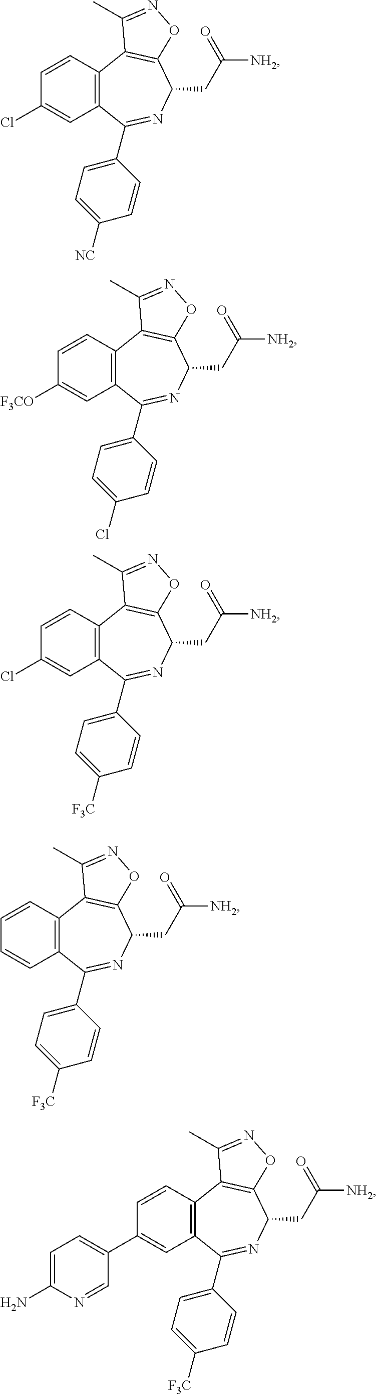

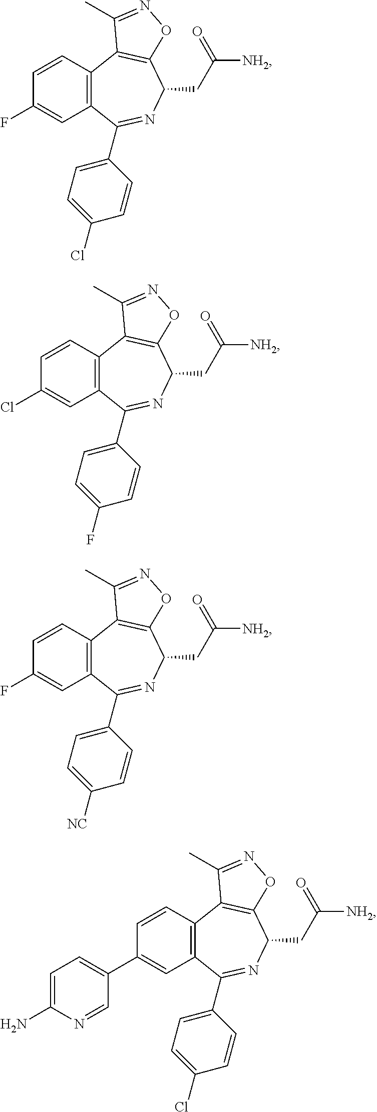

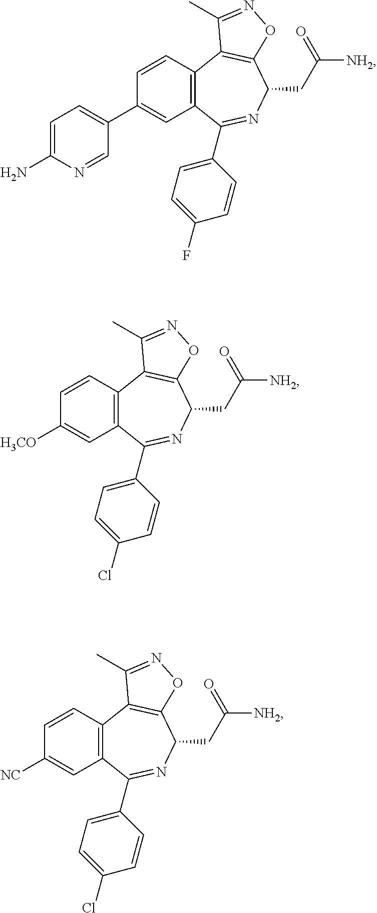 [SMILES: Cc1noc2c1-c1ccc(-c3ccc(N)nc3)cc1C(c1ccc(F)cc1)=N[C@H]2CC(N)=O ; COc1ccc2c(c1)C(c1ccc(Cl)cc1)=N[C@@H](CC(N)=O)c1onc(C)c1-2 ; Cc1noc2c1-c1ccc(C#N)cc1C(c1ccc(Cl)cc1)=N[C@H]2CC(N)=O]

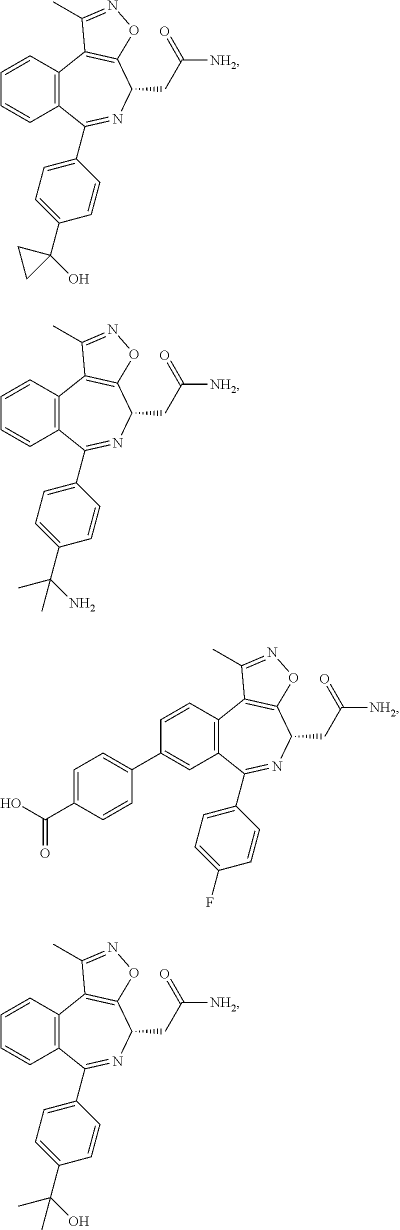 [SMILES: Cc1noc2c1-c1ccccc1C(c1ccc(C3(O)CC3)cc1)=N[C@H]2CC(N)=O ; Cc1noc2c1-c1ccccc1C(c1ccc(C(C)(C)N)cc1)=N[C@H]2CC(N)=O ; Cc1noc2c1-c1ccc(-c3ccc(C(=O)O)cc3)cc1C(c1ccc(F)cc1)=N[C@H]2CC(N)=O ; Cc1noc2c1-c1ccccc1C(c1ccc(C(C)(C)O)cc1)=N[C@H]2CC(N)=O]

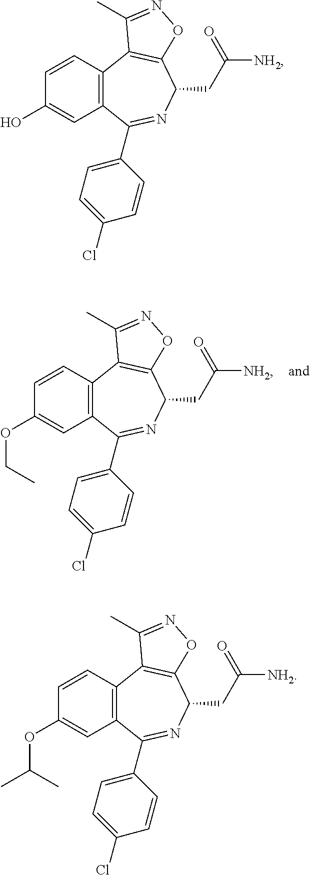 [SMILES: Cc1noc2c1-c1ccc(O)cc1C(c1ccc(Cl)cc1)=N[C@H]2CC(N)=O ; CCOc1ccc2c(c1)C(c1ccc(Cl)cc1)=N[C@@H](CC(N)=O)c1onc(C)c1-2 ; "and" ; Cc1noc2c1-c1ccc(OC(C)C)cc1C(c1ccc(Cl)cc1)=N[C@H]2CC(N)=O]

It will be appreciated that each of these compounds may be connected to a —Y—Z moiety, for example, as illustrated for generic structures Formula U, Formula V, and Formula W above.

In some embodiments, compounds of Formula U, Formula V, and Formula W may be selected from the group consisting of:

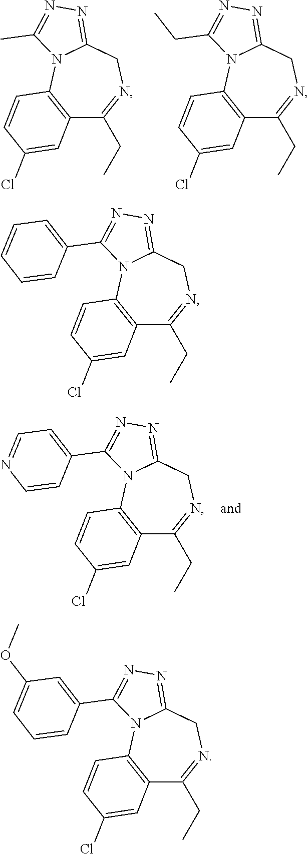

It will be appreciated that each of these compounds may be connected to a —Y—Z moiety, for example, as illustrated for generic structures Formula U, Formula V, and Formula W above.

In some embodiments, exemplary bromodomain ligands include compounds represented by the structures:

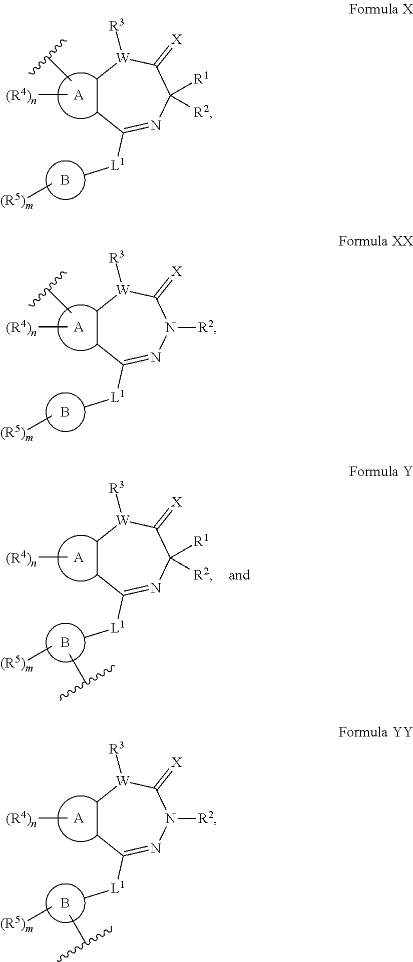

wherein:

Ring A is benzo, or a 5-6 membered fused heteroaryl ring having 1-3 heteroatoms independently selected from nitrogen, oxygen, or sulfur;

Ring B is a 3-7 membered saturated or partially unsaturated carbocyclic ring, phenyl, an 8-10 membered bicyclic saturated, partially unsaturated, phenyl or naphthyl ring, a 4-7 membered saturated or partially unsaturated heterocyclic ring having 1-2 heteroatoms independently selected from nitrogen, oxygen, and sulfur, a 5-6 membered monocyclic heteroaryl ring having 1-3 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;

L¹ is a covalent bond or an optionally substituted bivalent C₁₋₆ hydrocarbon chain wherein one or two methylene units is optionally replaced by —NR′—, —N(R′)C(O)—, —C(O)N(R′), —N(R′)SO₂—, —SO₂N(R′), —O—, —C(O)—, —OC(O)—, —C(O)O—, —S—, —SO— or —SO₂—;

R¹ is hydrogen, halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R′))N(R′)₂, —OC(O)R, —OC(O)N(R′)₂, or —(CH₂)ₚR^{x};

p is 0-3;

R^{x} is halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂;

R² is hydrogen, halogen, —CN, —SR, or optionally substituted C₁₋₆ aliphatic, or:

R¹ and R² are taken together with their intervening atoms to form an optionally substituted 3-7 membered saturated or partially unsaturated spiro-fused ring having 0-2 heteroatoms independently selected from nitrogen, oxygen, or sulfur;

each R is independently hydrogen or an optionally substituted group selected from C₁₋₆ aliphatic, phenyl, a 3-7 membered saturated or partially unsaturated carbocyclic ring, a 7-10 membered bicyclic saturated, partially unsaturated, phenyl or naphthyl ring, a 5-6 membered monocyclic heteroaryl ring having 1-3 heteroatoms independently selected from nitrogen, oxygen, and sulfur, a 4-7 membered saturated or partially unsaturated heterocyclic ring having 1-2 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;

each R′ is independently —R, —C(O)R, —C(S)R, —CO₂R, —C(O)N(R)₂, —C(S)N(R)₂, —S(O)R, —SO₂R, —SO₂N(R)₂, or two R′ on the same nitrogen are taken together with their intervening atoms to form an optionally substituted group selected from a 4-7 membered monocyclic saturated or partially unsaturated ring having 1-2 heteroatoms independently selected from nitrogen, oxygen, and sulfur, or a 7-12 membered bicyclic saturated, partially unsaturated, or aromatic fused ring having 1-3 heteroatoms independently selected from nitrogen, oxygen, and sulfur;

W is

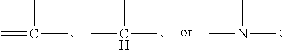

R³ is optionally substituted C₁₋₆ aliphatic;

X is oxygen or sulfur, or:

R³ and X are taken together with their intervening atoms to form an optionally substituted 5-membered heteroaryl ring having 1-4 heteroatoms independently selected from nitrogen, oxygen, or sulfur;

each of m and n is independently 0-4, as valency permits; and

each of R⁴ and R⁵ is independently —R, halogen, —OR, —SR, —N(R′)₂, —CN, —NO₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, or —OC(O)N(R′)₂.

In another embodiment, exemplary bromodomain ligands include compounds represented by the structures:

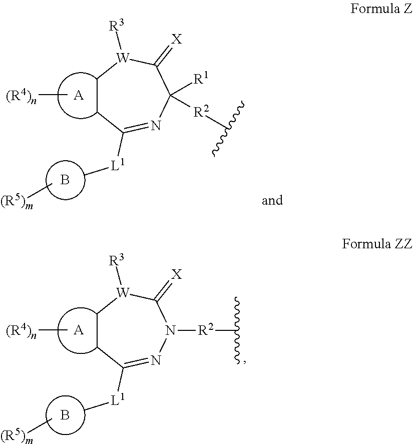

wherein:

Ring A is benzo, or a 5-6 membered fused heteroaryl ring having 1-3 heteroatoms independently selected from nitrogen, oxygen, or sulfur;

Ring B is a 3-7 membered saturated or partially unsaturated carbocyclic ring, phenyl, an 8-10 membered bicyclic saturated, partially unsaturated, phenyl or naphthyl ring, a 4-7 membered saturated or partially unsaturated heterocyclic ring having 1-2 heteroatoms independently selected from nitrogen, oxygen, and sulfur, a 5-6 membered monocyclic heteroaryl ring having 1-3 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;

L¹ is a covalent bond or an optionally substituted bivalent C₁₋₆ hydrocarbon chain wherein one or two methylene units is optionally replaced by —NR′—, —N(R′)C(O)—, —C(O)N(R′), —N(R′)SO₂—, —SO₂N(R′), —O—, —C(O)—, —OC(O)—, —C(O)O—, —S—, —SO— or —SO₂—;

R¹ is hydrogen, halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂, or —(CH₂)ₚR^{x};

p is 0-3;

R^{x} is halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂;

R² is a bond or optionally substituted C₁₋₆ aliphatic, or:

R¹ and R² are taken together with their intervening atoms to form an optionally substituted 3-7 membered saturated or partially unsaturated spiro-fused ring having 0-2 heteroatoms independently selected from nitrogen, oxygen, or sulfur;

each R is independently hydrogen or an optionally substituted group selected from C₁₋₆ aliphatic, phenyl, a 3-7 membered saturated or partially unsaturated carbocyclic ring, a 7-10 membered bicyclic saturated, partially unsaturated, phenyl, or naphthyl ring, a 5-6 membered monocyclic heteroaryl ring having 1-3 heteroatoms independently selected from nitrogen, oxygen, and sulfur, a 4-7 membered saturated or partially unsaturated heterocyclic ring having 1-2 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;

each R′ is independently —R, —C(O)R, —C(S)R, —CO₂R, —C(O)N(R)₂, —C(S)N(R)₂, —S(O)R, —SO₂R, —SO₂N(R)₂, or two R′ on the same nitrogen are taken together with their intervening atoms to form an optionally substituted group selected from a 4-7 membered monocyclic saturated or partially unsaturated ring having 1-2 heteroatoms independently selected from nitrogen, oxygen, and sulfur, or a 7-12 membered bicyclic saturated, partially unsaturated, or aromatic fused ring having 1-3 heteroatoms independently selected from nitrogen, oxygen, and sulfur;

W is

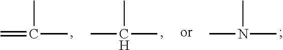

R³ is optionally substituted C₁₋₆ aliphatic;

X is oxygen or sulfur, or:

R³ and X are taken together with their intervening atoms to form an optionally substituted 5-membered heteroaryl ring having 1-4 heteroatoms independently selected from nitrogen, oxygen, or sulfur;

each of m and n is independently 0-4, as valency permits; and

each of R⁴ and R⁵ is independently —R, halogen, —OR, —SR, —N(R′)₂, —CN, —NO₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R′)₂, —C═NOR, —C(═N(R))N(R′)₂, —OC(O)R, or —OC(O)N(R′)₂.

For example, a compound of Formula X, Formula Y, or Formula Z may be selected from the group consisting of:

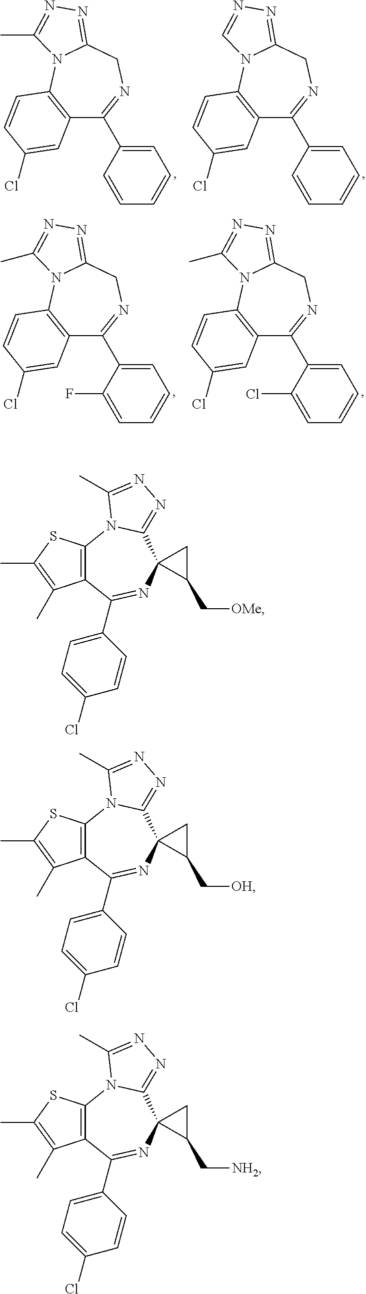

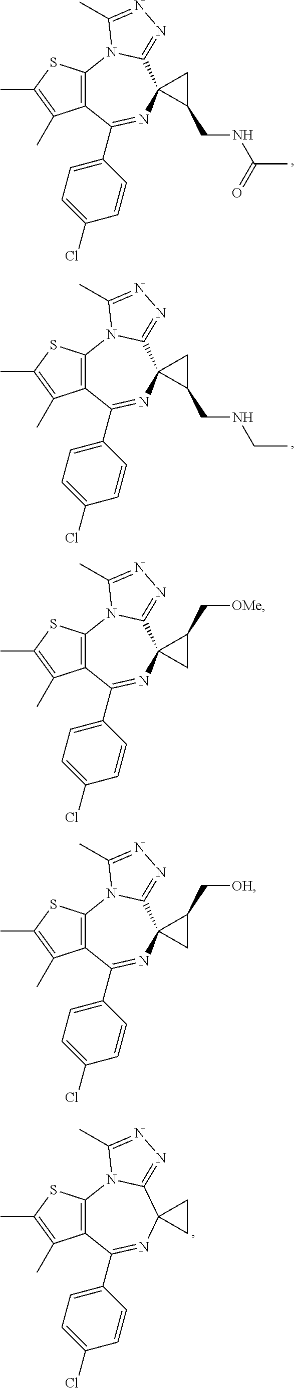

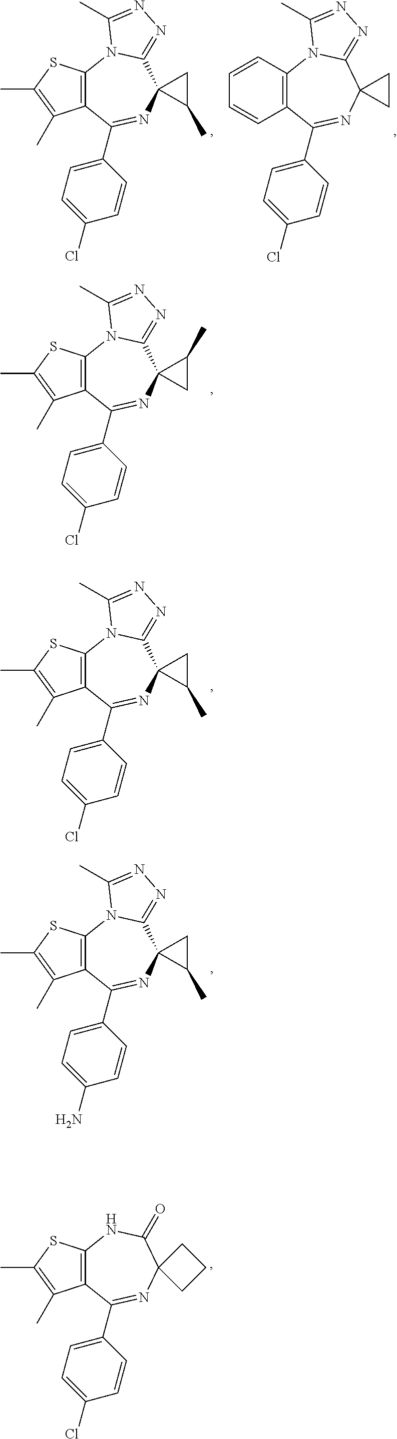

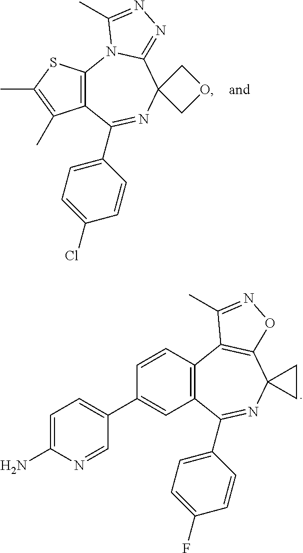 [SMILES: Cc1sc2c(c1C)C(c1ccc(Cl)cc1)=NC1(COC1)c1nnc(C)n1-2 ; "and" ; Cc1noc2c1-c1ccc(-c3ccc(N)nc3)cc1C(c1ccc(F)cc1)=NC21CC1]

It will be appreciated that each of these compounds may be connected to a —Y—Z moiety, for example, as illustrated for generic structures Formula X, Formula Y, and Formula Z above.

In some embodiments, a compound of Formula XX, Formula YY, or Formula ZZ may be selected from the group consisting of:

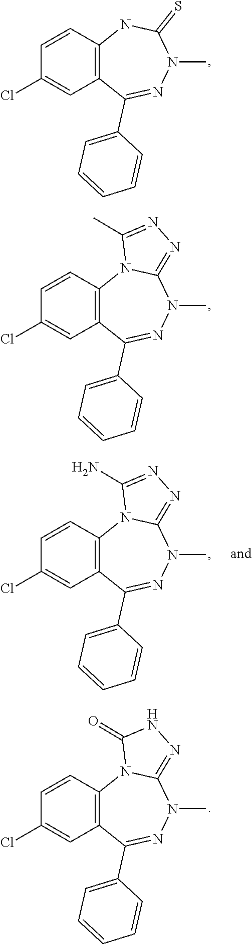

It will be appreciated that each of these compounds may be connected to a —Y—Z moiety, for example, as illustrated for generic structures Formula XX, Formula YY, and Formula ZZ above.

In another embodiment, exemplary bromodomain ligands include compounds represented by the structures:

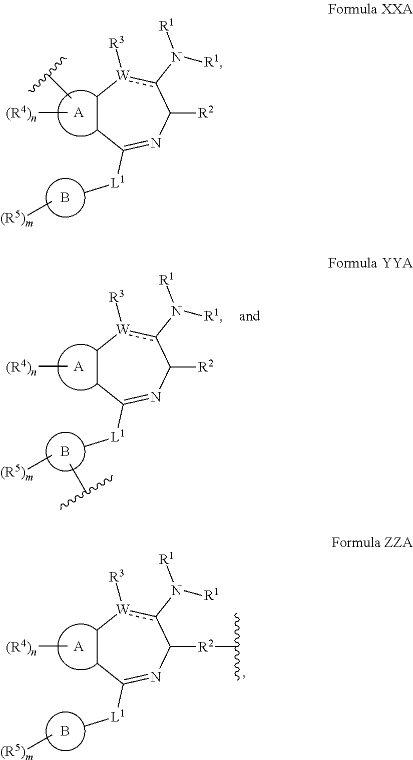

wherein:

Ring A is benzo, or a 5-6 membered fused heteroaryl ring having 1-3 heteroatoms independently selected from nitrogen, oxygen, or sulfur;

Ring B is a 3-7 membered saturated or partially unsaturated carbocyclic ring, phenyl, an 8-10 membered bicyclic saturated, partially unsaturated, phenyl, or naphthyl ring, a 4-7 membered saturated or partially unsaturated heterocyclic ring having 1-2 heteroatoms independently selected from nitrogen, oxygen, and sulfur, a 5-6 membered monocyclic heteroaryl ring having 1-3 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;

L¹ is a covalent bond or an optionally substituted bivalent C₁₋₆ hydrocarbon chain wherein one or two methylene units is optionally replaced by —NR′—, —N(R′)C(O)—, —C(O)N(R′), —N(R′)SO₂—, —SO₂N(R′), —O—, —C(O)—, —OC(O)—, —C(O)O—, —S—, —SO— or —SO₂—;

R¹ is independently hydrogen, halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)C(═N(R′))N(R′)₂, —C═NN(R′)₂, —C═NOR, —C(═N(R′))N(R′)₂, —OC(O)R, —OC(O)N(R′)₂, or —(CH₂)ₚR^{x};

p is 0-3;

R^{x} is halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R′))N(R′)₂, —OC(O)R, —OC(O)N(R′)₂;

R² is a bond, hydrogen, or optionally substituted C₁₋₆ aliphatic;

each R is independently hydrogen or an optionally substituted group selected from C₁₋₆ aliphatic, phenyl, a 3-7 membered saturated or partially unsaturated carbocyclic ring, a 7-10 membered bicyclic saturated, partially unsaturated, phenyl, or naphthyl ring, a 5-6 membered monocyclic heteroaryl ring having 1-3 heteroatoms independently selected from nitrogen, oxygen, and sulfur, a 4-7 membered saturated or partially unsaturated heterocyclic ring having 1-2 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;

each R′ is independently —R, —C(O)R, —C(S)R, —CO₂R, —C(O)N(R)₂, —C(S)N(R)₂, —S(O)R, —SO₂R, —SO₂N(R)₂, or two R′ on the same nitrogen are taken together with their intervening atoms to form an optionally substituted group selected from a 4-7 membered monocyclic saturated or partially unsaturated ring having 1-2 heteroatoms independently selected from nitrogen, oxygen, and sulfur, or a 7-12 membered bicyclic saturated, partially unsaturated, or aromatic fused ring having 1-3 heteroatoms independently selected from nitrogen, oxygen, and sulfur;

W is C or N;

R³ is optionally substituted C₁₋₆ aliphatic;

[symbol] is a single or double bond;

each of m and n is independently 0-4, as valency permits; and

each of R⁴ and R⁵ is independently —R, halogen, —OR, —SR, —N(R′)₂, —CN, —NO₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R′)₂, —C═NOR, —C(═N(R))N(R′)₂, —OC(O)R, or —OC(O)N(R′)₂.

For example, a compound of formula XXA, YYA, or ZZA may be:

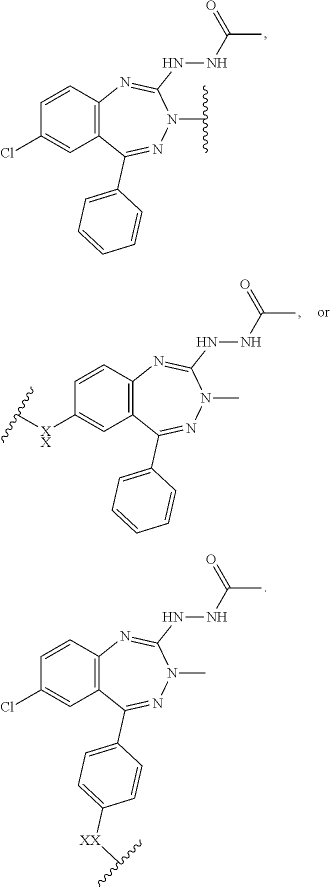

wherein XX may be a bond, C₁₋₆alkyl, —NR^{t}— (where t is H, phenyl, or C₁₋₆alkyl), —O—, or —S(O)_{w}— wherein w is 0, 1, or 2;

In yet another embodiment, exemplary bromodomain ligands include compounds represented by the structure:

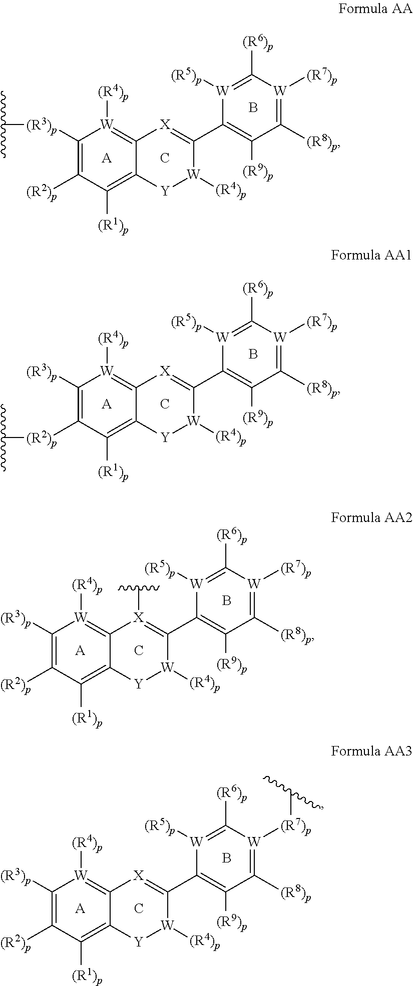

wherein:

X is selected from N and CH;

Y is CO;

R¹ and R³ are each independently selected from alkoxy and hydrogen;

R² is selected from alkoxy, alkyl, and hydrogen;

R⁶ and R⁸ are each independently selected from alkyl, alkoxy, chloride, and hydrogen;

R⁵ and R⁹ are each hydrogen;

R⁷ is selected from amino, hydroxyl, alkoxy, and alkyl substituted with a heterocyclyl;

R¹⁰ is hydrogen; or

two adjacent substituents selected from R⁶, R⁷, and R⁸ are connected to form a heterocyclyl;

each W is independently selected from C and N, wherein if W is N, then p is 0 or 1, and if W is C, then p is 1;

for W—(R¹⁰)ₚ, W is N and p is 1; and

for W—(R⁴)ₚ, W is C, p is 1 and R⁴ is H, or W is N and p is 0.

For example, in some embodiments, a compound of Formula AA may be:

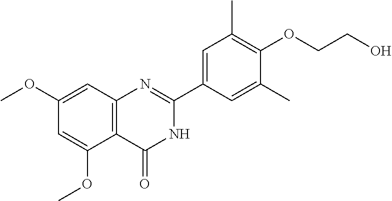 [SMILES: COc1cc(OC)c2c(=O)[nH]c(-c3cc(C)c(OCCO)c(C)c3)nc2c1]

(2-(4-(2-hydroxyethoxy)-3,5-dimethylphenyl)-5,7-dimethoxyquinazolin-4(3H)-one). It will be appreciated that this compound may be connected to a —Y—Z moiety, for example, as illustrated for generic structures Formula AA, Formula AA1, Formula AA2, and Formula AA3 above.

In still another embodiment, exemplary bromodomain ligands include compounds represented by the structures:

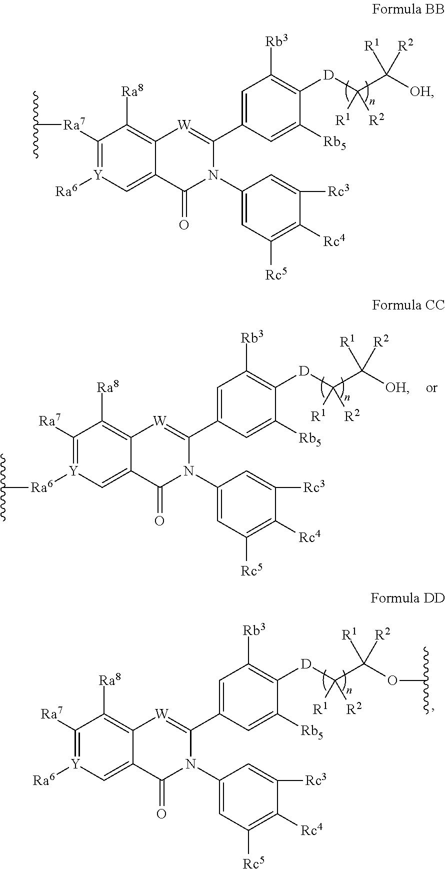

wherein:

Y and W are each independently selected from carbon and nitrogen;

Ra⁶ is selected from fluoride, hydrogen, C₁-C₃ alkoxy, cyclopropyloxy, SO₂R₃, SOR₃, and SR₃, wherein if Y is nitrogen then Ra⁶ is absent;

Ra⁷ is selected from hydrogen, fluoride, SO₂R₃, SOR₃, and SR₃;

Ra⁸ is selected from hydrogen, C₁-C₃ alkoxy, cyclopropyloxy, chloride, and bromide;

n is selected from 1, 2, or 3;

D is selected from O, NH, NR₁, S, or C;

Rb³ and Rb⁵ are independently selected from hydrogen and C₁-C₃ alkyl;

R_{C}³ and R_{C}⁵ are independently selected from hydrogen, C₁-C₃ alkyl, and cyclopropyl;

R_{C}⁴ is selected from F, Cl, Br, I, CF₃, C₁-C₆ alkyl, C₃-C₆ cycloalkyl, NHC(O)R⁴, NHSO₂R⁴, C(O)OR⁴, and

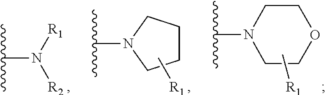

R¹, R′¹, R² and R′² are independently selected from hydrogen, fluoride, C₁-C₃ alkyl, and cyclopropyl, wherein R¹ and R² and/or R′¹ and R′² may be connected to form a 3-6 membered ring;

R³ is selected from C₁-C₃ alkyl and cyclopropyl; and

R⁴ is selected from hydrogen, C₁-C₄ alkyl, C₃-C₅ cycloalkyl, phenyl, and naphthyl, provided that if Ra′ or Ra^{b} is fluoride, then R_{C}⁴ is not bromide.

In some embodiments, a compound of Formula AA, Formula AAI, Formula AA2, Formula AA3, Formula BB, or Formula CC may be selected from the group consisting of: 3-(4-sec-butylphenyl)-2-(4-(2-hydroxyethoxy)-3,5-dimethylphenyl)quinazolin-4(3H)-one; 3-(4-bromophenyl)-2-(4-(2-hydroxyethoxy)-3,5-dimethylphenyl)quinazolin-4(3H)-one; 3-(4-sec-butylphenyl)-7-fluoro-2-(4-(2-hydroxyethoxy)-3,5-dimethylphenyl)quinazolin-4(3H)-one; 3-(4-sec-butylphenyl)-2-(4-(2-hydroxyethoxy)-3,5-dimethylphenyl)pyrido[4,3-d]pyrimidin-4(3H)-one; 3-(4-sec-butylphenyl)-2-(4-(2-hydroxyethoxy)phenyl)quinazolin-4(3H)-one; 3-(4-fluorophenyl)-2-(4-(2-hydroxyethoxy)-3,5-dimethylphenyl)quinazolin-4(3H)-one; 2-(4-(2-hydroxyethoxy)-3,5-dimethylphenyl)-3-(4-iodophenyl)quinazolin-4(3H)-one; 3-(4-sec-butylphenyl)-6-fluoro-2-(4-(2-hydroxyethoxy)-3,5-dimethylphenyl)quinazolin-4(3H)-one; 3-(4-chlorophenyl)-2-(4-(2-hydroxyethoxy)-3,5-dimethylphenyl)quinazolin-4(3H)-one; 2-(4-(2-hydroxyethoxy)-3,5-dimethylphenyl)-3-(4-(trifluoromethyl)phenyl)quinazolin-4(3H)-one; 3-(4-sec-butylphenyl)-2-(4-(2-hydroxyethoxy)-3,5-dimethylphenyl)-7-(methylsulfonyl)quinazolin-4(3H)-one; 3-(4-sec-butylphenyl)-2-(4-(2-hydroxyethoxy)-3,5-dimethylphenyl)-6-methoxyquinazolin-4(3H)-one; 3-(4-sec-butylphenyl)-2-(4-(2-hydroxyethoxy)-3,5-dimethylphenyl)-8-methoxyquinazolin-4(3H)-one; 3-(4-sec-butylphenyl)-2-(4-(2-hydroxyethoxy)-3,5-dimethylphenyl)-6-(methylsulfonyl)quinazolin-4(3H)-one; 3-(4-bromophenyl)-2-(4-(2-hydroxyethoxy)-3,5-dimethylphenyl)-6-methoxyquinazolin-4(3H)-one; 3-(4-bromophenyl)-2-(4-(2-hydroxyethoxy)-3,5-dimethylphenyl)-8-methoxyquinazolin-4(3H)-one; 2-(4-(2-hydroxyethoxy)-3,5-dimethylphenyl)-3-(4-isopropylphenyl)quinazolin-4(3H)-one; 3-(4-bromophenyl)-2-(4-(2-hydroxyethoxy)-3-methyiphenyl)quinazolin-4(3H)-one; 3-(4-bromophenyl)-8-chloro-2-(4-(2-hydroxyethoxy)-3,5-dimethylphenyl)quinazolin-4(3H)-one; 2-(4-(2-hydroxyethoxy)-3,5-dimethylphenyl)-3-(4-morpholinophenyl)quinazolin-4(3H)-one; 3-(4-tert-butylphenyl)-2-(4-(2-hydroxyethoxy)-3,5-dimethylphenyl)quinazolin-4(3H)-one; N-(4-(2-(4-(2-hydroxyethoxy)-3,5-dimethylphenyl)-4-oxoquinazolin-3(4H)-yl)phenyl)acetamide; N-(4-(2-(4-(2-hydroxyethoxy)-3,5-dimethylphenyl)-4-oxoquinazolin-3(4H)-yl)phenyl)isobutyramide; methyl 4-(2-(4-(2-hydroxyethoxy)-3,5-dimethylphenyl)-4-oxoquinazolin-3(4H)-yl)benzoate; 3-(4-cyclohexylphenyl)-2-(4-(2-hydroxyethoxy)-3,5-dimethylphenyl)quinazolin-4(3H)-one; N-(4-(2-(4-(2-hydroxyethoxy)-3,5-dimethylphenyl)-4-oxoquinazolin-3(4H)-yl)phenyl)formamide; 3-(4-aminophenyl)-2-(4-(2-hydroxyethoxy)-3,5-dimethylphenyl)quinazolin-4(3H)-one; N-(4-(2-(4-(2-hydroxyethoxy)-3,5-dimethylphenyl)-4-oxoquinazolin-3(4H)-yl)phenyl)methanesulfonamide; N-(4-(2-(4-(2-hydroxyethoxy)-3,5-dimethylphenyl)-4-oxoquinazolin-3(4H)-yl)phenyl)benzenesulfonamide; N-(4-(2-(4-(2-hydroxyethoxy)-3,5-dimethylphenyl)-4-oxoquinazolin-3(4H)-yl)phenyl)propane-2-sulfonamide; 3-(4-(dimethylamino)phenyl)-2-(4-(2-hydroxyethoxy)-3,5-dimethylphenyl)quinazolin-4(3H)-one; 3-(4-sec-butylphenyl)-2-(4-(2-hydroxyethoxy)-3-methylphenyl)quinazolin-4(3H)-one; 3-(4-chlorophenyl)-2-(4-(2-hydroxyethoxy)-3-methylphenyl)quinazolin-4(3H)-one; 3-(4-sec-butylphenyl)-2-(pyridin-3-yl)quinazolin-4(3H)-one; 3-(4-chlorophenyl)-2-(quinolin-3-yl)quinazolin-4(3H)-one; 3-(4-sec-butylphenyl)-2-(5-fluoropyridin-3-yl)quinazolin-4(3H)-one; 3-(4-chlorophenyl)-2-(6-chloropyridin-3-yl)quinazolin-4(3H)-one; 3-(4-sec-butylphenyl)-2-(6-chloropyridin-3-yl)quinazolin-4(3H)-one; 3-(4-sec-butylphenyl)-2-(6-methoxypyridin-3-yl)quinazolin-4(3H)-one; 2-(6-bromopyridin-3-yl)-3-(4-chlorophenyl)quinazolin-4(3H)-one; 2-(6-bromopyridin-3-yl)-3-(4-sec-butylphenyl)quinazolin-4(3H)-one; 3-(4-chlorophenyl)-2-(6-(diethylamino)pyridin-3-yl)quinazolin-4(3H)-one; 3-(4-sec-butylphenyl)-2-(6-(diethylamino)pyridin-3-yl)quinazolin-4(3H)-one; 3-(4-sec-butylphenyl)-2-(pyrimidin-5-yl)quinazolin-4(3H)-one; 3-(4-sec-butylphenyl)-2-(6-methylpyridin-3-yl)quinazolin-4(3H)-one; 3-(4-chlorophenyl)-2-(6-methylpyridin-3-yl)quinazolin-4(3H)-one; 3-(4-chlorophenyl)-2-(6-(piperidin-1-yl)pyridin-3-yl)quinazolin-4(3H)-one; 3-(4-sec-butylphenyl)-2-(6-(piperidin-1-yl)pyridin-3-yl)quinazolin-4(3H)-one; 3-(4-chlorophenyl)-2-(6-phenoxypyridin-3-yl)quinazolin-4(3H)-one; 3-(4-sec-butylphenyl)-2-(6-fluoropyridin-3-yl)quinazolin-4(3H)-one; 3-(4-sec-butylphenyl)-2-(6-phenoxypyridin-3-yl)quinazolin-4(3H)-one; 3-(4-chlorophenyl)-2-(6-(trifluoromethyl)pyridin-3-yl)quinazolin-4(3H)-one; 3-(4-sec-butylphenyl)-2-(6-(trifluoromethyl)pyridin-3-yl)quinazolin-4(3H)-one; 3-(4-sec-butylphenyl)-2-(6-phenylpyridin-3-yl)quinazolin-4(3H)-one; 3-(4-sec-butylphenyl)-2-(5-phenylpyridin-3-yl)quinazolin-4(3H)-one; 2-(5-bromopyridin-3-yl)-3-(4-sec-butylphenyl)quinazolin-4(3H)-one; 2-(5-bromopyridin-3-yl)-3-(4-chlorophenyl)quinazolin-4(3H)-one; 3-(4-sec-butylphenyl)-2-(5-(diethylamino)pyridin-3-yl)quinazolin-4(3H)-one; 3-(4-chlorophenyl)-2-(5-phenylpyridin-3-yl)quinazolin-4(3H)-one; 3-(4-chlorophenyl)-2-(5-(diethylamino)pyridin-3-yl)quinazolin-4(3H)-one; 3-(4-cyclopentylphenyl)-2-(6-methylpyridin-3-yl)quinazolin-4(3H)-one; 3-(4-sec-butylphenyl)-2-(6-(hydroxymethyl)pyridin-3-yl)quinazolin-4(3H)-one; 2-(6-methylpyridin-3-yl)-3-(4-(methylthio)phenyl)quinazolin-4(3H)-one; 3-(4-isopropylphenyl)-2-(6-methylpyridin-3-yl)quinazolin-4(3H)-one; N-(4-(2-(6-methylpyridin-3-yl)-4-oxoquinazolin-3(4H)-yl)phenyl)methanesulfonamide; 3-(4-sec-butylphenyl)-2-(6-(morpholinomethyl)pyridin-3-yl)quinazolin-4(3H)-one; 3-(4-cyclopropylphenyl)-2-(6-methylpyridin-3-yl)quinazolin-4(3H)-one; 3-(4-(dimethylamino)phenyl)-2-(6-methylpyridin-3-yl)quinazolin-4(3H)-one; 2-(6-chloropyridin-3-yl)-3-(4-cyclopropylphenyl)quinazolin-4(3H)-one; 3-(4-sec-butylphenyl)-2-(6-morpholinopyridin-3-yl)quinazolin-4(3H)-one; 3-(4-sec-butylphenyl)-2-(1H-indazol-5-yl)quinazolin-4(3H)-one; 3-(4-chlorophenyl)-2-(1H-indol-5-yl)quinazolin-4(3H)-one; 3-(4-sec-butylphenyl)-2-(1H-indol-5-yl)quinazolin-4(3H)-one; 3-(4-sec-butylphenyl)-2-(2-(hydroxymethyl)-1H-benzo[d]imidazol-6-yl)quinazolin-4(3H)-one; 2-(1H-indol-5-yl)-3-(4-(trifluoromethoxy)phenyl)quinazolin-4(3H)-one; 2-(1H-indol-5-yl)-3-(4-isopropylphenyl)quinazolin-4(3H)-one; 3-(4-chlorophenyl)-2-(1-(4-methoxyphenylsulfonyl)-1H-pyrrolo[2,3-b]pyridin-5-yl)quinazolin-4(3H)-one; 3-(4-chlorophenyl)-2-(1-(4-fluorophenylsulfonyl)-1H-pyrrolo[2,3-b]pyridin-5-yl)quinazolin-4(3H)-one; 3-(4-(dimethylamino)phenyl)-2-(1H-indol-5-yl)quinazolin-4(3H)-one; 3-(4-chlorophenyl)-2-(1-(phenylsulfonyl)-1H-pyrrolo[2,3-b]pyridin-5-yl)quinazolin-4(3H)-one; 3-(4-sec-butylphenyl)-2-(2-(hydroxymethyl)-1H-indol-5-yl)quinazolin-4(3H)-one; 3-(4-chlorophenyl)-2-(1-methyl-1H-indol-5-yl)quinazolin-4(3H)-one; 3-(4-cyclopentylphenyl)-2-(1H-indol-5-yl)quinazolin-4(3H)-one; 3-(4-chlorophenyl)-2-(1H-indol-6-yl)quinazolin-4(3H)-one; 3-(4-chlorophenyl)-2-(1H-indol-7-yl)quinazolin-4(3H)-one; 3-(4-sec-butylphenyl)-2-(1H-indol-6-yl)quinazolin-4(3H)-one; 3-(4-sec-butylphenyl)-2-(1H-indol-7-yl)quinazolin-4(3H)-one; 3-(4-chlorophenyl)-2-(1H-indol-4-yl)quinazolin-4(3H)-one; and 3-(4-sec-butylphenyl)-2-(1H-indol-4-yl)quinazolin-4(3H)-one. It will be appreciated that each of these compounds may be connected to a —Y—Z moiety, for example, as illustrated for generic structures Formula AA, Formula AA1, Formula AA2, Formula AA3, Formula BB, Formula CC, and Formula DD.

In yet another embodiment, exemplary bromodomain ligands include compounds represented by the structure:

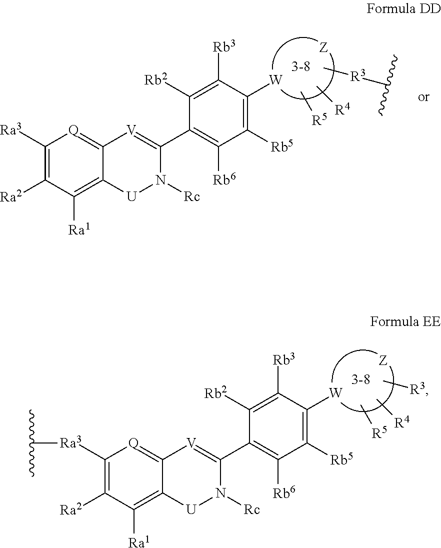

wherein:

Q and V are independently selected from CH and nitrogen;

U is selected from C═O, C═S, SO₂, S═O, SR¹, CR¹R², CR¹OR², CR¹SR²;

R¹ and R² are independently selected from hydrogen and C₁-C₆ alkyl;

Rc is selected from hydrogen, C₁-C₆ alkyl, and C₃-C₆ cycloalkyl;

Ra¹, Ra², and Ra³ are independently selected from hydrogen, C₁-C₆ alkyl, C₁-C₆ alkenyl, C₁-C₆ alkynyl, C₁-C₆ alkoxy, halogen, amino, amide, hydroxyl, heterocycle, and C₃-C₆ cycloalkyl, wherein Ra¹ and Ra² and/or Ra² and Ra³ may be connected to form a cycloalkyl or a heterocycle;

Rb² and Rb⁶ are independently selected from hydrogen, halogen, C₁-C₆ alkyl, C₁-C₆ alkenyl, C₃-C₆ cycloalkyl, hydroxyl, and amino;

Rb³ and Rb⁵ are independently selected from hydrogen, halogen, C₁-C₆ alkyl, C₁-C₆ alkoxy, C₃-C₆ cycloalkyl, hydroxyl, and amino, wherein Rb² and Rb³ and/or Rb⁵ and Rb⁶ may be connected to form a cycloalkyl or a heterocycle;

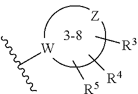

represents a 3-8 membered ring system wherein: W is selected from carbon and nitrogen; Z is selected from CR⁶R⁷, NR⁸, oxygen, sulfur, —S(O)—, and —SO₂—; said ring system being optionally fused to another ring selected from cycloalkyl, heterocycle, and phenyl, and wherein said ring system is optionally selected from rings having the structures:

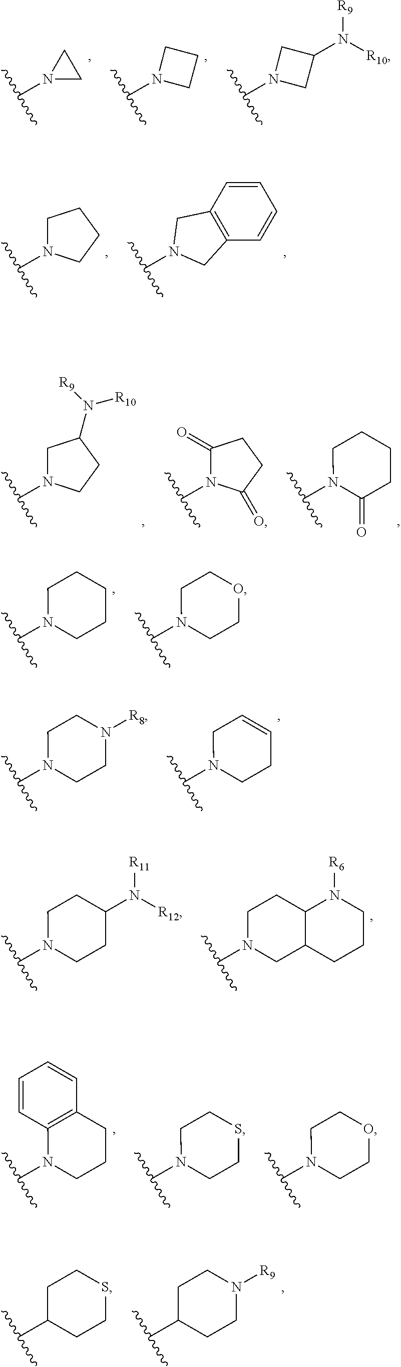

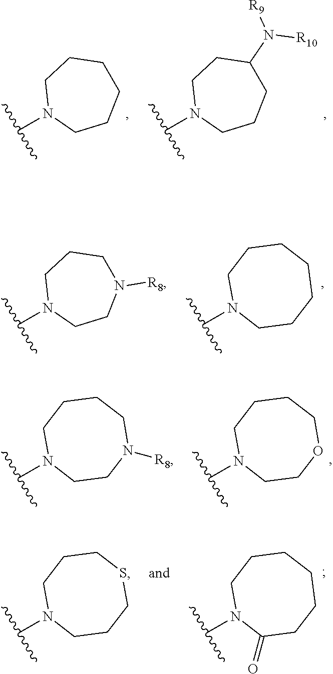

R³, R⁴, and R⁵ are independently selected from hydrogen, C₁-C₆ alkyl, C₁-C₆ alkenyl, C₁-C₆ alkynyl, C₁-C₆ alkoxy, C₃-C₆ cycloalkyl, phenyl, naphthyl, aryloxy, hydroxyl, amino, amide, oxo, —CN, and sulfonamide;

R⁶ and R⁷ are independently selected from hydrogen, C₁-C₆ alkyl, C₁-C₆ alkenyl, C₁-C₆ alkynyl, C₃-C₆ cycloalkyl, phenyl, naphthyl, halogen, hydroxyl, —CN, amino, and amido; and

R⁸ is selected from hydrogen, C₁-C₆ alkyl, C₁-C₆ alkenyl, C₁-C₆ alkynyl, acyl, and C₃-C₆ cycloalkyl; and

R⁹, R¹⁰, R¹¹, and R¹² are independently selected from hydrogen, C₁-C₆ alkyl, C₁-C₆ alkenyl, C₁-C₆ alkynyl, C₃-C₆ cycloalkyl, phenyl, naphthyl, heterocycle, hydroxyl, sulfonyl, and acyl.

In still another embodiment, exemplary bromodomain ligands include compounds represented by the structure:

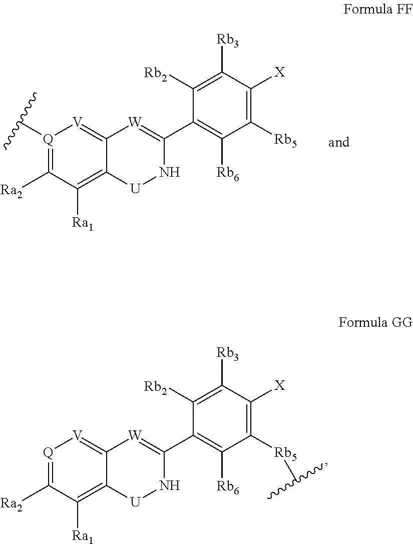

wherein:

Q is selected from N and CRa³;

V is selected from N and CRa⁴;

W is selected from N and CH;

U is selected from C═O, C═S, SO₂, S═O, and SR¹;

X is selected from OH, SH, NH₂, S(O)H, S(O)₂H, S(O)₂NH₂, S(O)NH₂, NHAc, and NHSO₂Me;

Ra¹, Ra³, and Ra³ are independently selected from hydrogen, C₁-C₆ alkyl, C₁-C₆ alkoxy, C₃-C₆ cycloalkyl, and halogen;

Ra² is selected from hydrogen, C₁-C₆ alkyl, C₁-C₆ alkoxy, C₃-C₆ cycloalkyl, amino, amide, and halogen;

Rb² and Rb⁶ are independently selected from hydrogen, methyl and fluorine;

Rb³ and Rb⁵ are independently selected from hydrogen, halogen, C₁-C₆ alkyl, C₃-C₆ cycloalkyl, and C₁-C₆ alkoxy; and

Rb² and Rb³ and/or Rb⁵ and Rb⁶ may be connected to form a cycloalkyl or a heterocycle, provided that at least one of Ra¹, Ra², Ra³, and Ra⁴ is not hydrogen.

In yet another embodiment, exemplary bromodomain ligands include compounds represented by the structure:

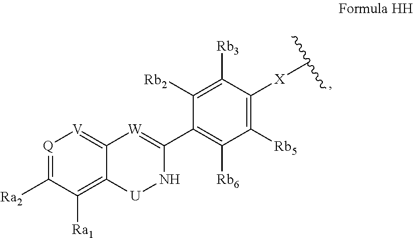

wherein:

Q is selected from N and CRa³;

V is selected from N and CRa⁴;

W is selected from N and CH;

U is selected from C═O, C═S, SO₂, S═O, and SR¹;

X is selected from OH, SH, NH₂, S(O)H, S(O)₂H, S(O)₂NH₂, S(O)NH₂, NHAc, and NHSO₂Me;

Ra¹, Ra³, and Ra³ are independently selected from hydrogen, C₁-C₆ alkyl, C₁-C₆ alkoxy, C₃-C₆ cycloalkyl, and halogen;

Ra² is selected from hydrogen, C₁-C₆ alkyl, C₁-C₆ alkoxy, C₃-C₆ cycloalkyl, amino, amide, and halogen;

Rb² and Rb⁶ are independently selected from hydrogen, methyl and fluorine;

Rb³ and Rb⁵ are independently selected from hydrogen, halogen, C₁-C₆ alkyl, C₃-C₆ cycloalkyl, and C₁-C₆ alkoxy; and

Rb² and Rb³ and/or Rb⁵ and Rb⁶ may be connected to form a cycloalkyl or a heterocycle, provided that at least one of Ra¹, Ra², Ra³, and Ra^{o} is not hydrogen.

The following are hereby incorporated by reference in their entirety: Zeng et al. J. Am. Chem. Soc. (2005) 127, 2376-2377; Chung et al. J. Med. Chem. (2012) 55, 576-586; Filippakopoulos et al. Bioorg. Med. Chem. (2012) 20, 1878-1886; U.S. Pat. No. 8,053,440, by Hansen; U.S. Patent Publication No. 2008/0188467, by Wong et al.; U.S. Patent Publication No. 2012/0028912; International Patent Publication Nos. WO/2010/123975, WO/2010/106436, WO/2010/079431, WO/2009/158404, and WO/2008/092231, by Hansen et al.; International Patent Publication Nos. WO/2012/075456 and WO/2012/075383, by Albrecht et al.; International Patent Publication Nos. WO/2007/084625 and WO/2006/083692, by Zhou et al.

In another aspect, exemplary bromodomain ligands include fused heterocyclic systems represented by the structures:

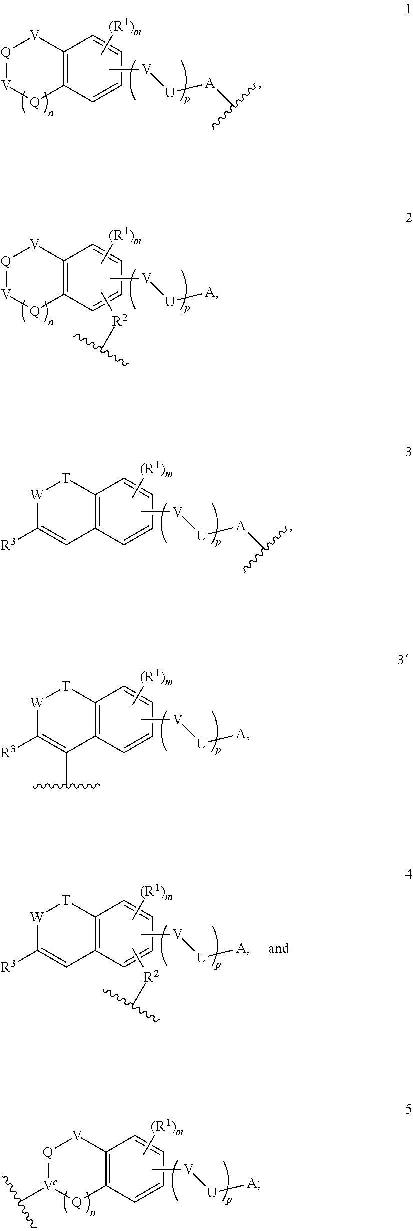

wherein:

V is independently selected, for each occurrence, from the group consisting of NH, S, N(C₁₋₆alkyl), O, or CR⁴R⁴;

Q is independently selected, for each occurrence, from the group consisting of C(O), C(S), C(N), SO₂, or CR⁴R⁴;

U is independently selected from the group consisting of a bond, C(O), C(S), C(N), SO₂, or CR⁴R⁴

W and T are independently selected from the group consisting of NH, N(C₁₋₆alkyl), O, or Q;

V^{C} is selected from the group consisting of N, SH or CR⁴;

A is selected from the group consisting of aliphatic, cycloalkyl, heterocyclic, phenyl, naphthyl, heteroaryl or bicyclic moiety, wherein the cycloalkyl, heterocyclic, phenyl, naphthyl, heteroaryl, or bicyclic moiety is optionally substituted with one, two, three, four or more groups represented by R⁴;

R¹ is independently selected, for each occurrence, from the group consisting of hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆ alkyl, nitro, cyano, CF₃, —OCF₃, —C(O)OC₁₋₆ alkyl, —OS(O)₂C₁₋₄ alkyl, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;

R² is selected from the group consisting of —O—, amino, C₁₋₆alkyl, —O—C₁₋₆alkyl-, hydroxylC₁₋₆ alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆alkyl-, —OS(O)₂C₁₋₄alkyl-, —OS(O)₂—, —S—C₁₋₆alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;

R³ is selected from the group consisting of hydrogen or C₁₋₆alkyl;

R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋₆alkyl, —NH—C₁₋₆ alkyl, —N(C₁₋₆ alkyl)C₁₋₆ alkyl, nitro, cyano, CF₃, —OCF₃, —C(O)OC₁₋₆ alkyl, —C(O)NHC₁₋₆alkyl, —C(O)NH₂ or —OS(O)₂C₁₋₄alkyl;

m is selected from the group consisting of 0, 1, 2, or 3;

n is selected from the group consisting of 0, 1, or 2; and

p is selected from the group consisting of 0 or 1.

For example, compounds of Formula 1, Formula 2 or Formula 5 may be selected from the group consisting of:

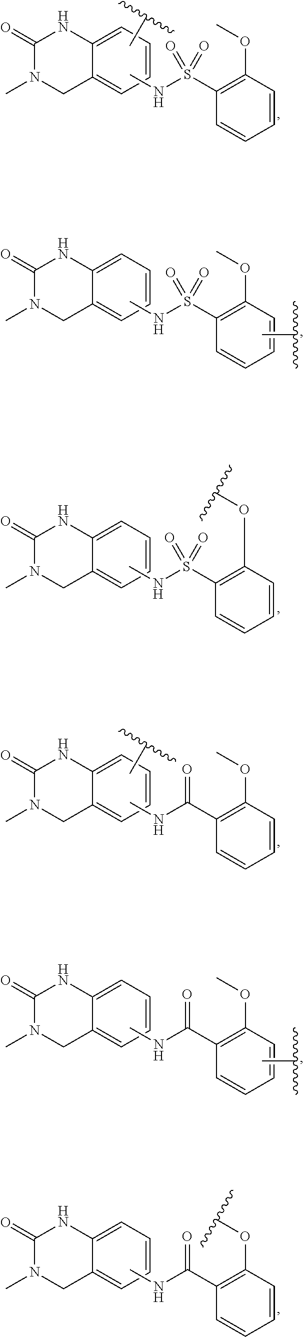

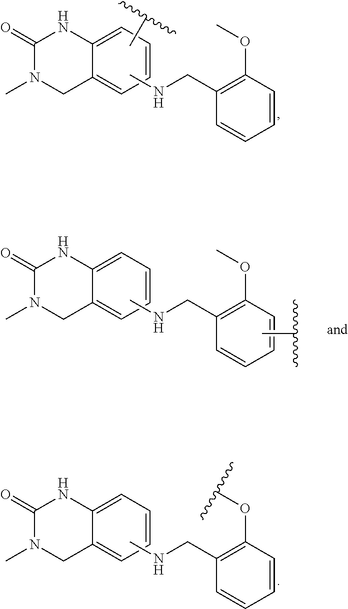

In a further example, compounds of Formula 1, Formula 2 or Formula 5 may be selected from the group consisting of:

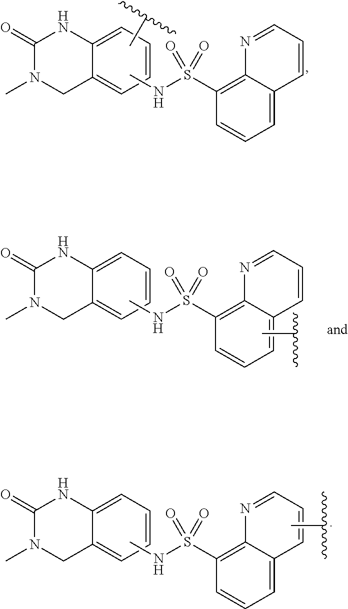

For example, compounds of Formula 3, Formula 3′ or Formula 4 may be selected from the group consisting of:

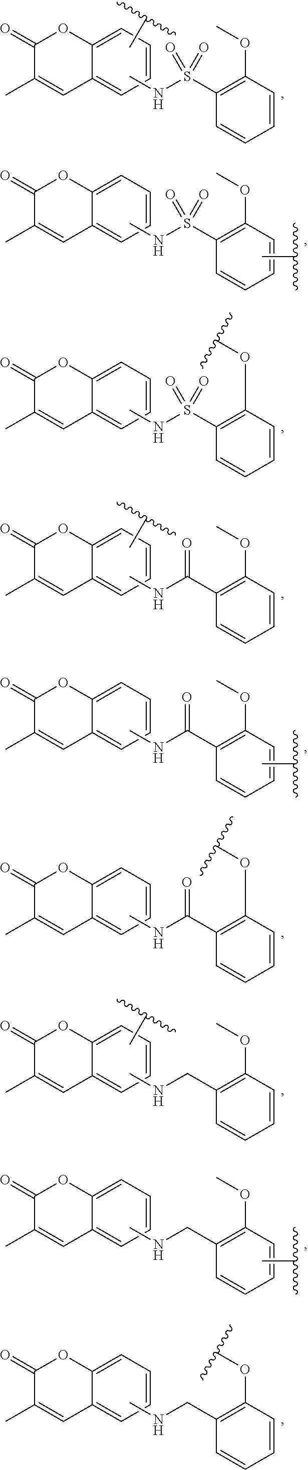

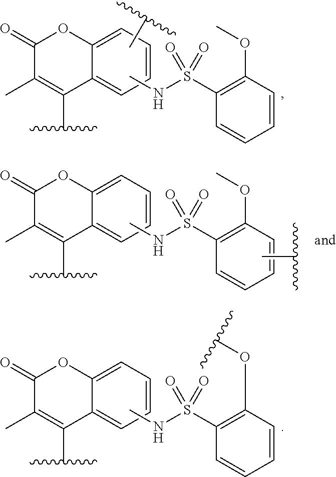

In another embodiment, bromodomain ligands include fused heterocyclic systems represented by the structures:

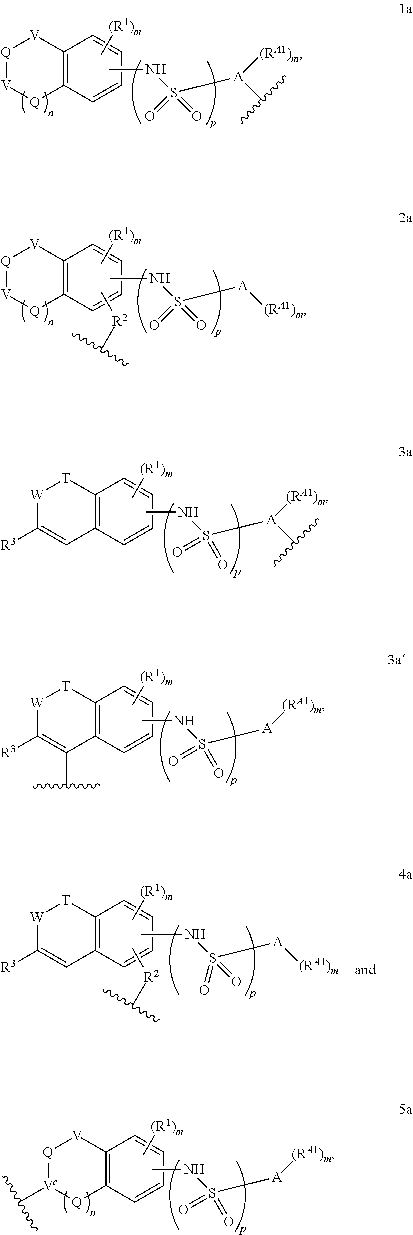

wherein:

V is independently selected, for each occurrence, from the group consisting of NH, S, N(C₁₋₆alkyl), O, or CR⁴R⁴;

Q is independently selected, for each occurrence, from the group consisting of C(O), C(S), C(N), SO₂, or CR⁴R⁴;

W and T are independently selected from the group consisting of NH, N(C₁₋₆alkyl), O, or Q;

V^{C} is selected from the group consisting of N, SH or CR⁴;

A is a ring selected from the group consisting of: phenyl, a 5-6 membered cycloalkyl, a 5-6 membered heteroaryl having 1, 2 or 3 heteroatoms each selected from S, N or O, and a 4-7 membered heterocycle having 1, 2 or 3 heteroatoms each selected from N or O;

R^{A1} is R¹; or two R^{A1} substituents may be taken together with the atoms to which they are attached to form phenyl, a 5-6 membered heteroaryl having 1, 2 or 3 heteroatoms each selected from S, N or O, and a 4-7 membered heterocycle having 1, 2 or 3 heteroatoms each selected from N or O;

R¹ is independently selected, for each occurrence, from the group consisting of hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆alkyl, nitro, cyano, CF₃, —OCF₃, —C(O)OC₁₋₆alkyl, —OS(O)₂C₁₋₄alkyl, phenyl, naphthyl, phenyloxy, benzyloxy or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;

R² is selected from the group consisting of —O—, amino, C₁₋₆alkyl, —O—C₁₋₆alkyl-, hydroxylC₁₋₆ alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆alkyl-, —OS(O)₂C₁₋₄alkyl-, —OS(O)₂—, —S—C₁₋₆alkyl-, phenyl, naphthyl, phenyloxy, benzyloxy or phenylmethoxy, wherein C₁₋₆alkyl phenyl, and naphthylare optionally substituted by one two or three substituents selected from the group consisting of hydroxyl, halogen, oxo, C₁₋₆alkyl, amino, or nitro;

R³ is selected from the group consisting of hydrogen or C₁₋₆alkyl;

R⁴ is independently selected, for each occurrence, selected from the group consisting of hydrogen, hydroxyl, oxo, imino, amino, halo, C₁₋₆alkyl, cycloalkyl, phenyl, naphthyl, heterocyclyl, —O—C₁₋₆ alkyl, —NH—C₁₋₆ alkyl, —N(C₁₋₆ alkyl)C₁₋₆ alkyl, nitro, cyano, CF₃, —OCF₃, —C(O)OC₁₋₆alkyl, —C(O)NHC₁₋₆alkyl, —C(O)NH₂ or —OS(O)₂C₁₋₄alkyl;

m is independently selected, for each occurrence, selected from the group consisting of 0, 1, 2, or 3;

n is selected from the group consisting of 0, 1, or 2; and

p is selected from the group consisting of 0 or 1.

A person of skill in the art appreciates that certain substituents may, in some embodiments, result in compounds that may have some instability and hence would be less preferred.

For example, compounds of Formula 1a, Formula 2a or Formula 5a may be selected from the group consisting of:

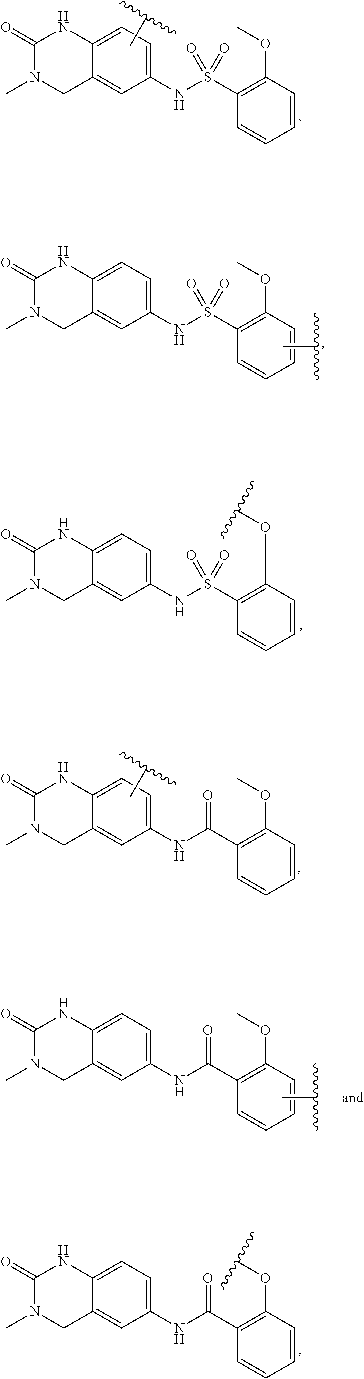

For example, compounds of Formula 3a or Formula 4a may be selected from the group consisting of:

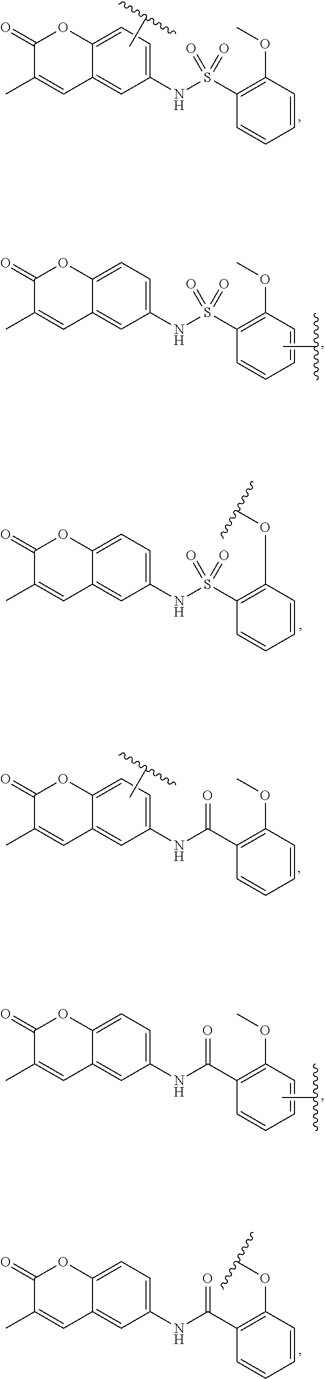

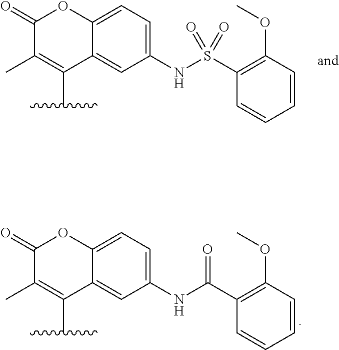 [SMILES: *c1c(C)c(=O)oc2ccc(NS(=O)(=O)c3ccccc3OC)cc12 ; *c1c(C)c(=O)oc2ccc(NC(=O)c3ccccc3OC)cc12]

In a further embodiment, bromodomain ligands include fused heterocyclic systems represented by the structures:

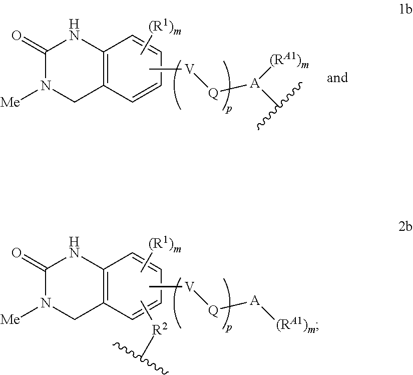

wherein:

V is selected from the group consisting of a NH, S, N(C₁₋₆alkyl), O, or CR⁴R⁴;

Q is selected from the group consisting of a bond, C(O), C(S), C(N), SO₂, or CR⁴R⁴;

A is a ring selected from the group consisting of: phenyl, a 5-6 membered cycloalkyl, a 5-6 membered heteroaryl having 1, 2 or 3 heteroatoms each selected from S, N or O, and a 4-7 membered heterocycle having 1, 2 or 3 heteroatoms each selected from N or O;

R^{A1} is R¹; or two R^{A1} substituents may be taken together with the atoms to which they are attached to form phenyl, a 5-6 membered heteroaryl having 1, 2 or 3 heteroatoms each selected from S, N or O, and a 4-7 membered heterocycle having 1, 2 or 3 heteroatoms each selected from N or O;

R¹ is independently selected, for each occurrence, from the group consisting of hydroxyl, halo, C₁₋₆ alkyl, hydroxyC₁₋₆ alkyl, aminoC₁₋₆ alkyl, haloC₁₋₆ alkyl, C₁₋₆ alkoxy, haloC₁₋₆ alkoxy, acylaminoC₁₋₆alkyl, nitro, cyano, CF₃, —OCF₃, —C(O)OC₁₋₆alkyl, —OS(O)₂C₁₋₄alkyl, —S(C₁₋₄alkyl)C(O)R′, phenyl, naphthyl, phenyloxy, benzyloxy, or phenylmethoxy, wherein C₁₋₆alkyl, phenyl, and napththyl are optionally substituted by one two or three substituents selected from the group consisting of hydroxyl, halogen, oxo, C₁₋₆alkyl, amino, or nitro;

R² is selected from the group consisting of —O—, amino, C₁₋₆alkyl, —O—C₁₋₆alkyl-, hydroxylC₁₋₆ alkyl, aminoC₁₋₆ alkyl, haloC₁₋₆alkyl, haloC₁₋₆ alkoxy, acylaminoC₁₋₆ alkyl, —C(O)—, —C(O)O—, —C(O)NC₁₋₆alkyl-, —OS(O)₂C₁₋₄alkyl-, —OS(O)₂—S(C₁₋₄alkyl)C(O)R″—, —S—C₁₋₆alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;

R³ is selected from the group consisting of hydrogen or C₁₋₆alkyl;

R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋₆alkyl, —NH—C₁₋₆alkyl, —N(C₁₋₆ alkyl)C₁₋₆ alkyl, nitro, cyano, CF₃, —OCF₃, —C(O)OC₁₋₆ alkyl, —C(O)NHC₁₋₆alkyl, —C(O)NH₂ or —OS(O)₂C₁₋₄alkyl;

R′ is independently selected, for each occurrence, from the group consisting of hydroxyl, amino, thio, phenyl, naphthyl, or C₁₋₆alkyl, wherein C₁₋₆alkyl, phenyl, and naphthyl are optionally substituted by one two or three substituents selected from the group consisting of hydroxyl, halogen, oxo, C₁₋₆alkyl, amino, or nitro;

R″ is independently selected, for each occurrence, from the group consisting of —O—, amino, thio, phenyl, naphthyl, or C₁₋₆alkyl, wherein C₁₋₆alkyl, phenyl, and naphthyl are optionally substituted by one two or three substituents selected from the group consisting of hydroxyl, halogen, oxo, C₁₋₆alkyl, amino, or nitro;

m is independently selected, for each occurrence, from the group consisting of 0, 1, 2, or 3;

n is selected from the group consisting of 0, 1, or 2; and

p is selected from the group consisting of 0 or 1.

Exemplary bromodomain ligands include fused heterocyclic systems represented by the structures:

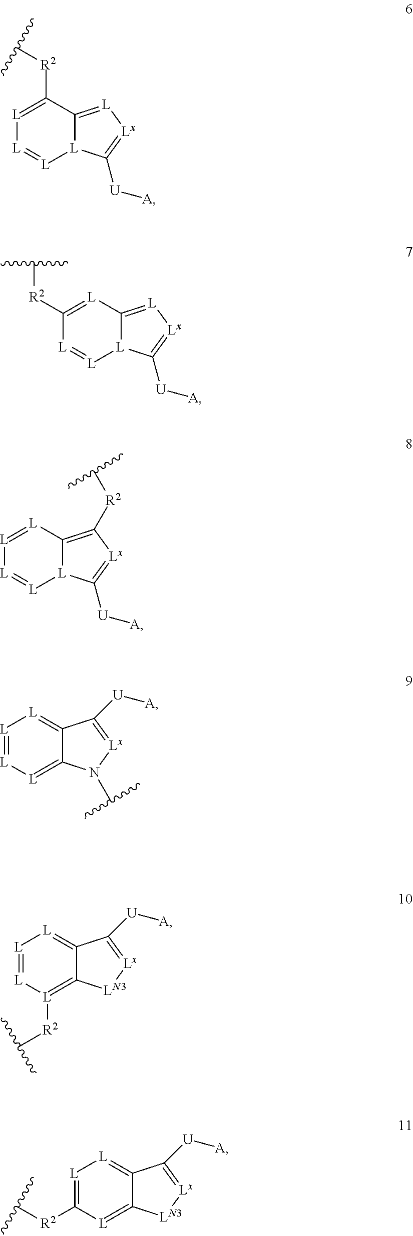

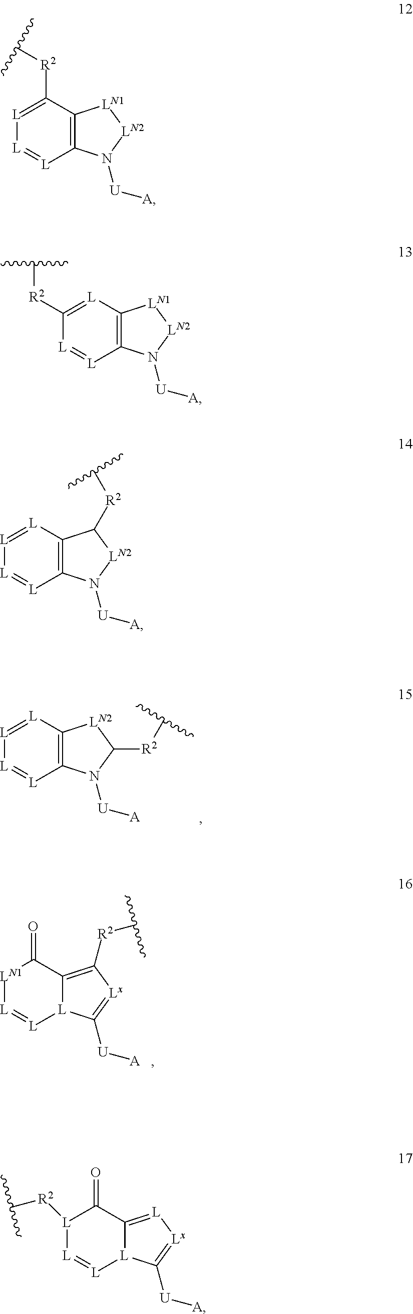

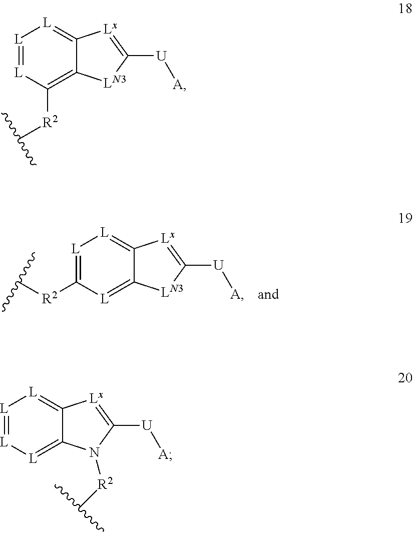

wherein:

L and L^{x} are independently selected, for each occurrence, from the group consisting of N, CH, and CR′;

L^{N1} and L^{N2} are independently selected from the group consisting of CH₂, CHR¹, CR¹R¹, NH, and N(C₁₋₆alkyl); wherein C₁₋₆alkyl is optionally substituted by one two or three substituents selected from the group consisting of hydroxyl, halogen, oxo, C₁₋₆alkyl, amino, or nitro;

L^{N3} is selected from the group consisting of O, S, NH, and N(C₁₋₆alkyl); wherein C₁₋₆alkyl is optionally substituted by one two or three substituents selected from the group consisting of hydroxyl, halogen, oxo, C₁₋₆alkyl, amino, or nitro;

U is independently selected from the group consisting of a bond, C(O), C(S), C(N), SO₂, or CR⁴R⁴;

A is selected from the group consisting of aliphatic, cycloalkyl, heterocyclic, phenyl, naphthyl, heteroaryl, or bicyclic moiety, wherein the cycloalkyl, heterocyclic, phenyl, naphthyl, heteroaryl, or bicyclic moiety is optionally substituted with one, two, three, four or more groups represented by R⁴;

R¹ is independently selected, for each occurrence, from the group consisting of hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆ alkyl, nitro, cyano, CF₃, —OCF₃, —C(O)OC₁₋₆ alkyl, —OS(O)₂C₁₋₄ alkyl, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;

R² is selected from the group consisting of —O—, amino, C₁₋₆alkyl, —O—C₁₋₆alkyl-, hydroxylC₁₋₆ alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆alkyl-, —OS(O)₂C₁₋₄alkyl-, —OS(O)₂—, —S—C₁₋₆alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;

R³ is selected from the group consisting of hydrogen or C₁₋₆alkyl; and

R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋6alkyl, —NH—C₁₋₆alkyl, —N(C₁₋₆ alkyl)C₁₋₆ alkyl, nitro, cyano, CF₃, —OCF₃, —C(O)OC₁₋₆ alkyl, —C(O)NHC₁₋₆alkyl, —C(O)NH₂ or —OS(O)₂C₁₋₄alkyl.

For example, compounds of Formula 6, 7, 8, 9, 10, 11, 12, 13, 14, 15, 16 and 17 may be selected from the group consisting of:

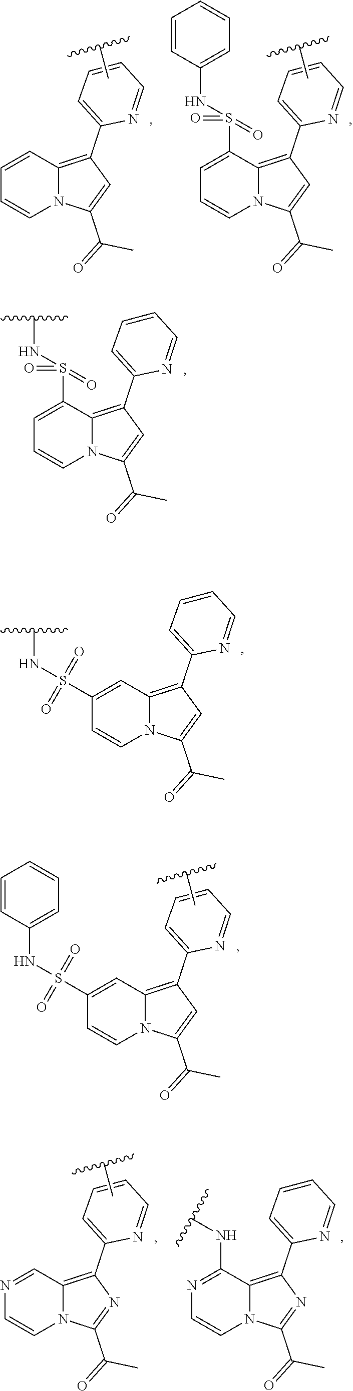

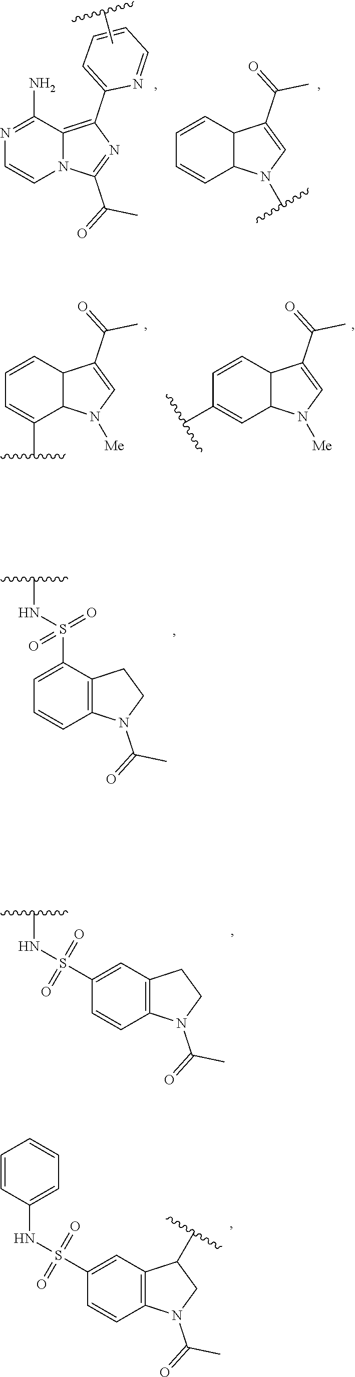

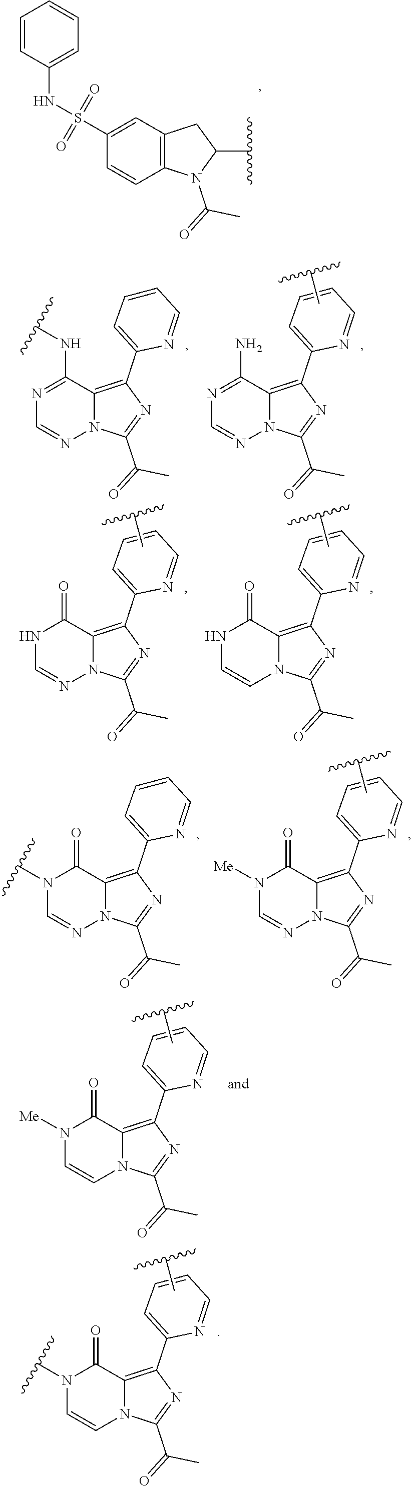

In certain other embodiments, the ligand is one of the compounds listed in Table 1 below or a pharmaceutically acceptable salt thereof, wherein the connector attachment point may be understood to be on

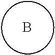

### or A.

TABLE 1

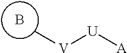

| Com- | | | | |
|---|---|---|---|---|
| pound | | | | |
| No, | B | V | U | A |
| I-1 | 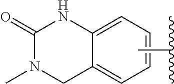 | NH | SO₂ | 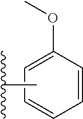 |
| I-2 | 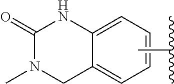 | NH | SO₂ | 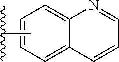 |
| I-3 | 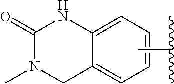 | NH | SO₂ | 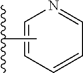 |
| I-4 | 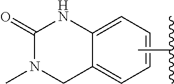 | NH | SO₂ | 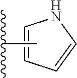 |
| I-5 | 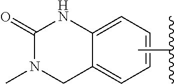 | NH | SO₂ | 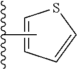 |
| I-6 | 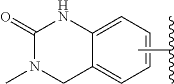 | NH | SO₂ | 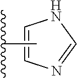 |
| I-7 | 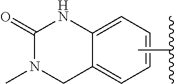 | NH | SO₂ | 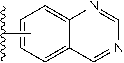 |
| I-8 | 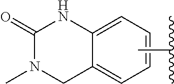 | NH | SO₂ | 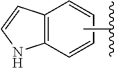 |
| I-9 | 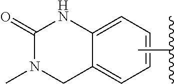 | NH | SO₂ | 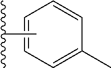 |
| I-10 | 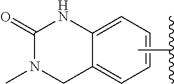 | NH | SO₂ | 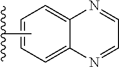 |
| I-11 | 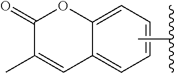 | NH | SO₂ | 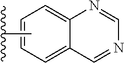 |
| I-12 | 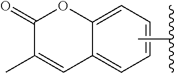 | NH | C(O) | 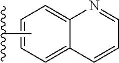 |
| I-13 | 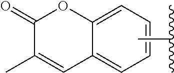 | O | C(O) | 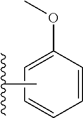 |
| I-14 | 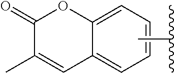 | CH₂ | CH₂ | 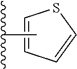 |
| I-15 | 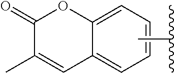 | O | CH₂ | 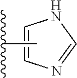 |
| I-16 | 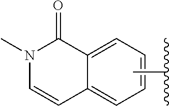 | NH | CH₂ | 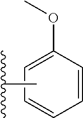 |
| I-17 | 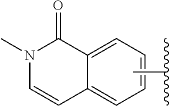 | NH | C(O) | 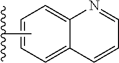 |
| I-18 | 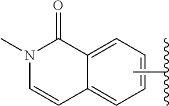 | NH | SO₂ | 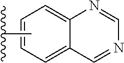 |
| I-19 | 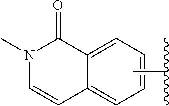 | NH | SO₂ | 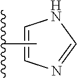 |
| I-20 | 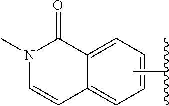 | NH | C(O) | 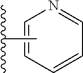 |
| I-21 | 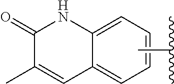 | CH₂ | CH₂ | 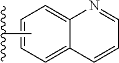 |
| I-22 | 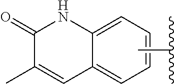 | O | C(O) | 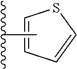 |
| I-23 | 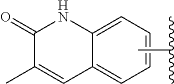 | NH | SO₂ | 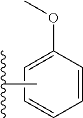 |
| I-24 | 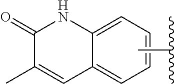 | NH | CH₂ | 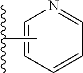 |
| I-25 | 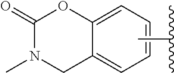 | NH | SO₂ | 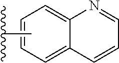 |
| I-26 | 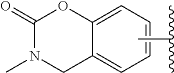 | CH₂ | CH₂ | 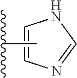 |
| I-27 | 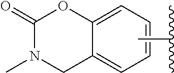 | NH | C(O) | 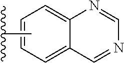 |
| I-28 | 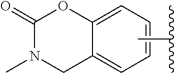 | NH | SO₂ | 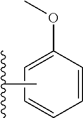 |
| I-29 | 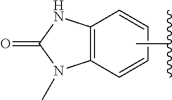 | NH | SO₂ | 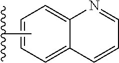 |
| I-30 | 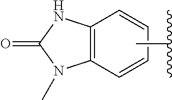 | NH | C(O) | 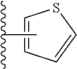 |
| I-31 | 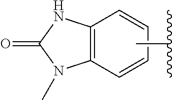 | NH | CH₂ | 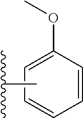 |
| I-32 | 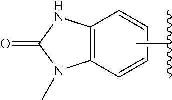 | CH₂ | CH₂ | 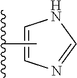 |
| I-33 | 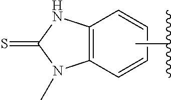 | O | C(O) | 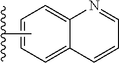 |
| I-34 | 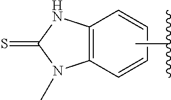 | NH | SO₂ | 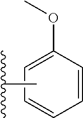 |
| I-35 | 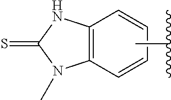 | NH | C(O) | 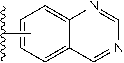 |
| I-36 | 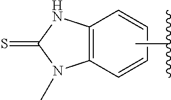 | CH₂ | CH₂ | 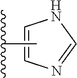 |
| I-37 | 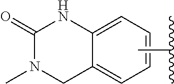 | NH | C(O) | 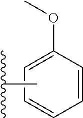 |
| I-38 | 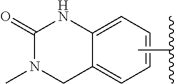 | SO₂ | NH | 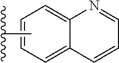 |
| I-39 | 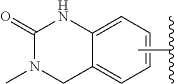 | O | C(O) | 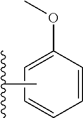 |
| I-40 | 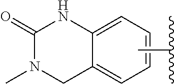 | C(O) | NH | 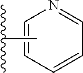 |
| I-41 | 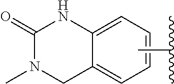 | CH₂ | CH₂ | 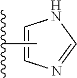 |
| I-42 | 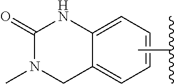 | NH | CH₂ | 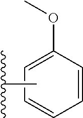 |
| I-43 | 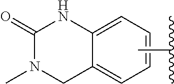 | CH₂ | NH | 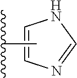 |
| I-44 | 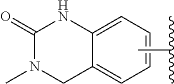 | O | CH₂ | 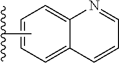 |

One of ordinary skill in the art will appreciate that certain substituents may, in some embodiments, result in compounds that may have some instability and hence would be less preferred.

### C) Connectors

The connector moieties Y¹, Y², Y³ and Y⁴ of Formulas I, II, III and IV may, in some embodiments, be the same or different. For example, connector moieties are independently contemplated herein.

In some embodiments, a monomer may comprise a connector that joins the ligand moiety with the linker element. In some instances, such connectors do not have significant binding or other affinity to an intended target. However, in certain embodiments, a connector may contribute to the affinity of a ligand moiety to a target.

In some embodiments, a connector element may be used to connect the linker element to the ligand moiety. In some instances, a connector element may be used to adjust spacing between the linker element and the ligand moiety. In some cases, the connector element may be used to adjust the orientation of the linker element and the ligand moiety. In certain embodiments, the spacing and/or orientation the linker element relative to the ligand moiety can affect the binding affinity of the ligand moiety (e.g., a pharmacophore) to a target. In some cases, connectors with restricted degrees of freedom are preferred to reduce the entropic losses incurred upon the binding of a multimer to its target biomolecule. In some embodiments, connectors with restricted degrees of freedom are preferred to promote cellular permeability of the monomer.

In some embodiments, the connector element may be used for modular assembly of monomers. For example, in some instances, a connector element may comprise a functional group formed from reaction of a first and second molecule. In some cases, a series of ligand moieties may be provided, where each ligand moiety comprises a common functional group that can participate in a reaction with a compatible functional group on a linker element. In some embodiments, the connector element may comprise a spacer having a first functional group that forms a bond with a ligand moiety and a second functional group that forms a bond with a linker element.

Contemplated connecters may be any acceptable (e.g. pharmaceutically and/or chemically acceptable) bivalent linker that, for example, does not interfere with multimerization of the disclosed monomers. For instance, such linkers may be substituted or unsubstituted C₁-C₁₀ alkylene, substituted or unsubstituted cycloalkylene, substituted or unsubstituted phenyl or naphthyl, substituted or unsubstituted heteroaryl, acyl, sulfone, phosphate, ester, carbamate, or amide. Contemplated connectors may include polymeric connectors, such a polyethylene glycol (e.g.,

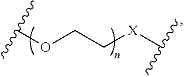

where n is 1, 2, 3, 4, 5, 6, 7, 8, 9, 10, 11, 12, 13, 14, 15, 16, 17, 18, 19, or 20, and X is O, S, NH, or —C(O)—) or other pharmaceutically acceptable polymers. For example, contemplated connectors may be a covalent bond or a bivalent C₁₋₁₀ saturated or unsaturated, straight or branched, hydrocarbon chain, wherein one, two, or three or four methylene units of L are optionally and independently replaced by cyclopropylene, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, phenyl, or a mono or bicyclic heterocycle ring. In some embodiments, a connector may be from about 7 atoms to about 13 atoms in length, or about 8 atoms to about 12 atoms, or about 9 atoms to about 11 atoms in length. For purposes of counting connector length when a ring is present in the connector group, the ring is counted as three atoms from one end to the other. In another embodiment, a connector moiety may maximally span from about 5 Å to about 50 Å, in some embodiments about 5 Å to about 2 in some embodiments about 20 Å to about 50 Å, and in some embodiments about 6 Å to about 15 Å in length.

In another embodiment, for the above-identified benzodiazepine compounds, there are e.g., three possible attachment points for the connector element: the phenyl ether, the amino group, or the chloro position of the chlorophenyl ring. As seen below, the connector element may be identified as a Y group in benzodiazepine-connector 1 A, benzodiazepine-connector 2 B, and benzodiazepine-connector 3 D:

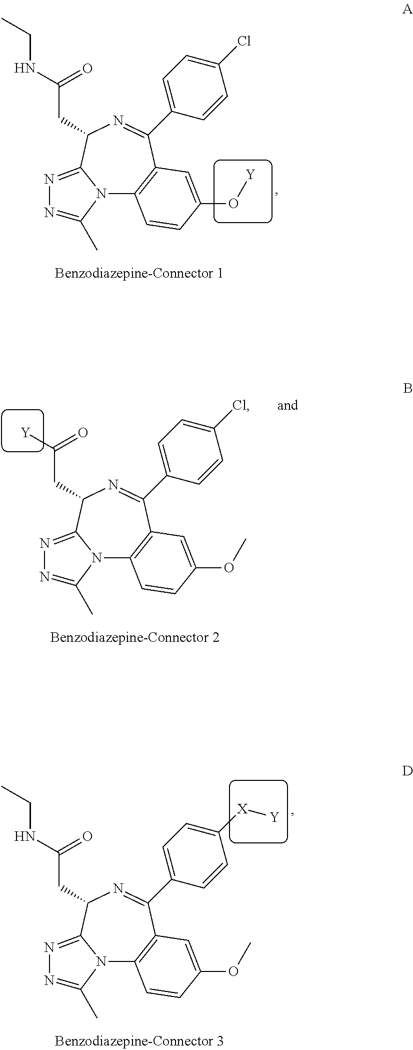

where X=CH₂, S, O, or NH.

For example, Y¹, Y², Y³ and Y⁴ may be Y as described above in connector 1 A, connector 2 B, or connector 3 D.

The synthetic route in Scheme Xa illustrates a general method for preparing benzodiazepine-connector 1 derivatives. The method involves attaching the desired substituents to the phenol core. Benzodiazepine 1 can be prepared following procedures described below. The desired Y group attached at the 4-position of the phenol can be installed by reacting benzodiazepine 1 with the appropriate electrophile 2 to provide 3 (benzodiazepine-connector 1 derivative). For example, Scheme Xa provides for a connector Y (e.g. Y¹, Y², Y³ or Y⁴).

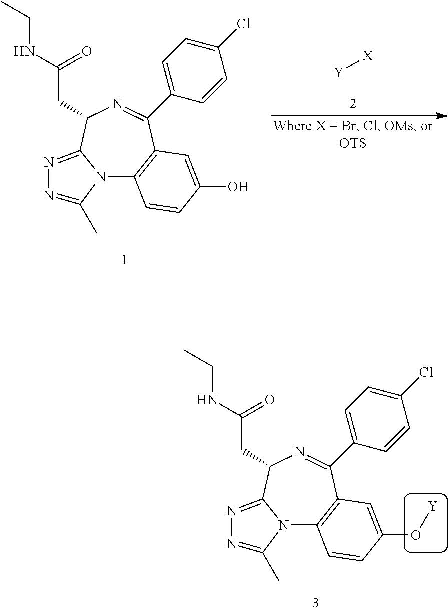

For example, Y may be selected from the group consisting of:

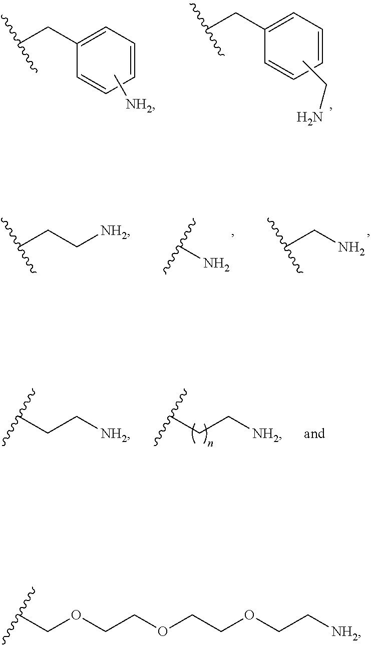

wherein n is 0, 1, 2, 3, 4 or 5.

Additional examples for 2 and Y can be found in Table A, seen below:

TABLE A

| No. | 2 | -Y |
|---|---|---|
| 1 | 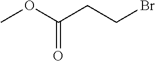 | 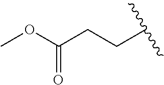 [SMILES: *CCC(=O)OC] |
| 2 | 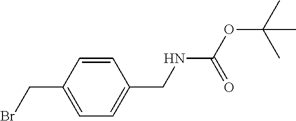 [SMILES: CC(C)(C)OC(=O)NCc1ccc(CBr)cc1] | 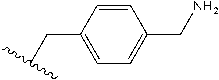 [SMILES: *Cc1ccc(CN)cc1] |
| 3 | 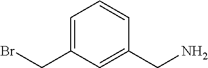 | 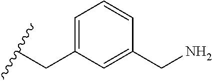 [SMILES: *Cc1cccc(CN)c1] |
| 4 | 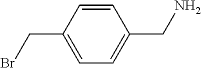 | 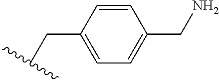 [SMILES: *Cc1ccc(CN)cc1] |
| 5 | 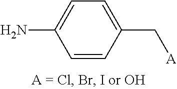 | 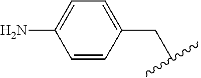 [SMILES: *Cc1ccc(N)cc1] |
| 6 | 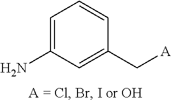 | 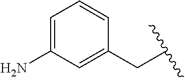 [SMILES: *Cc1cccc(N)c1] |
| 7 | 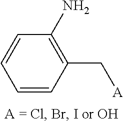 | 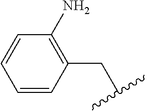 [SMILES: *Cc1ccccc1N] |
| 8 | 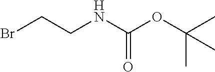 | 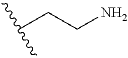 [SMILES: *CCN] |
| 9 | 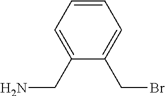 | 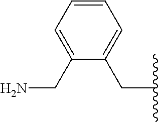 [SMILES: *Cc1ccccc1CN] |
| 10 | 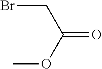 | 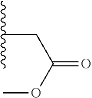 [SMILES: *CC(=O)OC] |
| 11 | 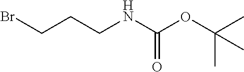 | 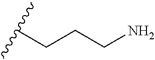 [SMILES: *CCCN] |
| 12 | 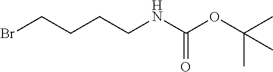 | 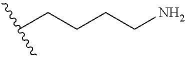 [SMILES: *CCCCN] |

The following table (Table U) indicates exemplary benzodiazepine-connector 1 derivatives (e.g., 3 of Scheme Xa) that include a ligand moiety (X) and a connector (Y). It is understood that such derivatives can be modified to include a pharmacophore (Z) such as provided for herein.

TABLE U

| No. | Compound Structure (X-Y) |
|---|---|
| 1 | 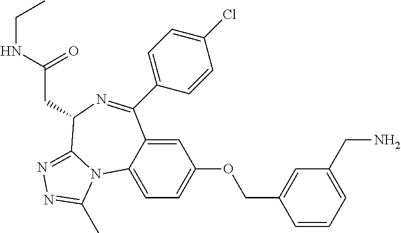 [SMILES: CCNC(=O)C[C@@H]1N=C(c2ccc(Cl)cc2)c2cc(OCc3cccc(CN)c3)ccc2-n2c(C)nnc21] |
| 2 | 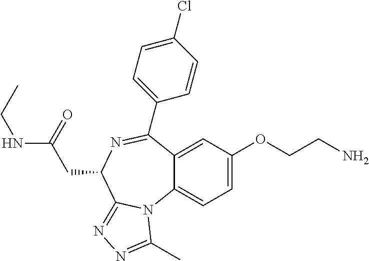 [SMILES: CCNC(=O)C[C@@H]1N=C(c2ccc(Cl)cc2)c2cc(OCCN)ccc2-n2c(C)nnc21] |
| 3 | 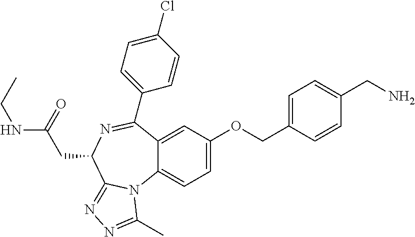 [SMILES: CCNC(=O)C[C@@H]1N=C(c2ccc(Cl)cc2)c2cc(OCc3ccc(CN)cc3)ccc2-n2c(C)nnc21] |
| 4 | 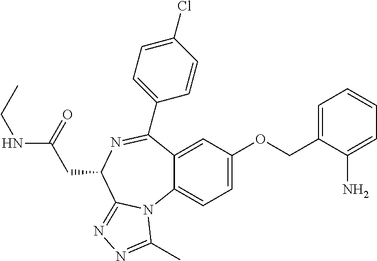 [SMILES: CCNC(=O)C[C@@H]1N=C(c2ccc(Cl)cc2)c2cc(OCc3ccccc3N)ccc2-n2c(C)nnc21] |
| 5 | 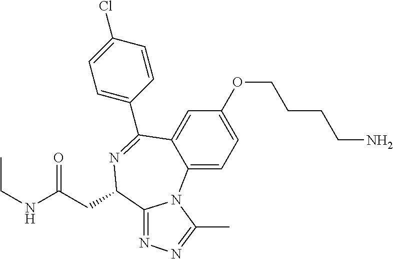 [SMILES: CCNC(=O)C[C@@H]1N=C(c2ccc(Cl)cc2)c2cc(OCCCCN)ccc2-n2c(C)nnc21] |
| 6 | 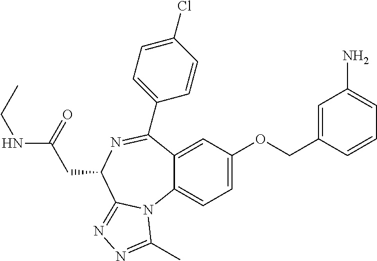 [SMILES: CCNC(=O)C[C@@H]1N=C(c2ccc(Cl)cc2)c2cc(OCc3cccc(N)c3)ccc2-n2c(C)nnc21] |
| 7 | 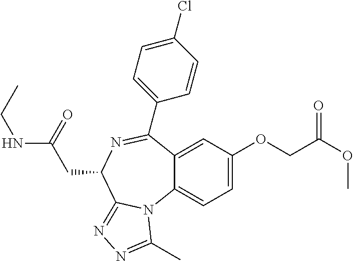 [SMILES: CCNC(=O)C[C@@H]1N=C(c2ccc(Cl)cc2)c2cc(OCC(=O)OC)ccc2-n2c(C)nnc21] |
| 8 | 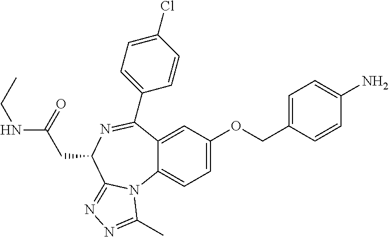 [SMILES: CCNC(=O)C[C@@H]1N=C(c2ccc(Cl)cc2)c2cc(OCc3ccc(N)cc3)ccc2-n2c(C)nnc21] |
| 9 | 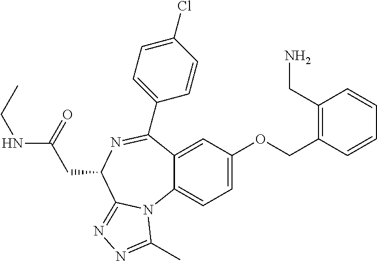 [SMILES: CCNC(=O)C[C@@H]1N=C(c2ccc(Cl)cc2)c2cc(OCc3ccccc3CN)ccc2-n2c(C)nnc21] |
| 10 | 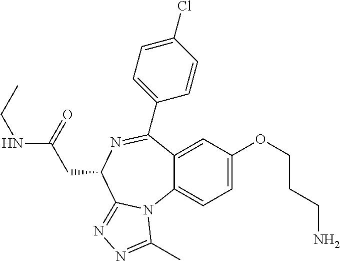 [SMILES: CCNC(=O)C[C@@H]1N=C(c2ccc(Cl)cc2)c2cc(OCCCN)ccc2-n2c(C)nnc21] |

Any free amino group seen in the Y examples of Table A above may be functionalized further to include additional functional groups, e.g., a benzoyl moiety.

In another embodiment, the attachment point identified in A (benzodiazepine-connector 1) may be further elaborated to incorporate not only the connector moiety (Y), but also the linker (Z), as represented by:

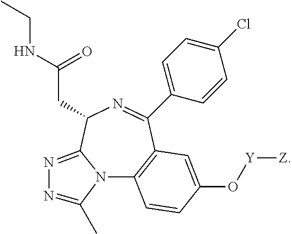

The Y—Z moiety may be formed from direct attachment of Y—Z to the phenyl ether, or the Y—Z moiety may be formed from the further functionalization of any free amino group seen in the Y examples of Table A above to include the linker moiety (Z). Examples of Y—Z groups having a boronic acid linker (Z) can be found in Table A″, seen below. It is clear from the linker section described above that a first monomer that has a boronic acid linker may be capable of forming a multimer with a second monomer that has a diol linker.

TABLE A”

| No. | Reagent | —Y—Z |
|---|---|---|
| 1 (direct attachment of Y—Z) | 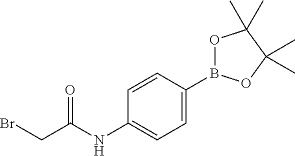 [SMILES: CC1(C)OB(c2ccc(NC(=O)CBr)cc2)OC1(C)C] | 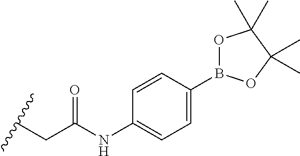 [SMILES: *CC(=O)Nc1ccc(B2OC(C)(C)C(C)(C)O2)cc1] |
| 2 (direct attachment of Y—Z) | 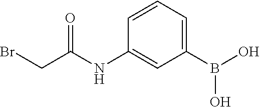 [SMILES: O=C(CBr)Nc1cccc(B(O)O)c1] | 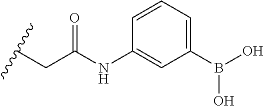 [SMILES: *CC(=O)Nc1cccc(B(O)O)c1] |
| 3 (direct attachment of Y—Z) | 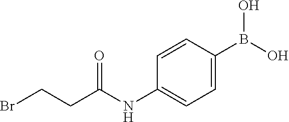 [SMILES: O=C(CCBr)Nc1ccc(B(O)O)cc1] | 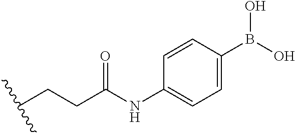 [SMILES: *CCC(=O)Nc1ccc(B(O)O)cc1] |
| 4 (direct attachment of Y—Z) | 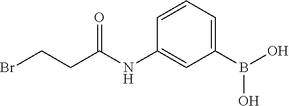 [SMILES: O=C(CCBr)Nc1cccc(B(O)O)c1] | 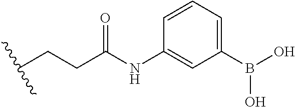 [SMILES: *CCC(=O)Nc1cccc(B(O)O)c1] |
| 5 (direct attachment of Y—Z) | 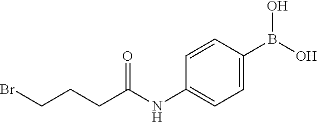 [SMILES: O=C(CCCBr)Nc1ccc(B(O)O)cc1] | 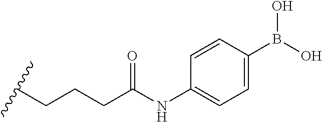 [SMILES: *CCCC(=O)Nc1ccc(B(O)O)cc1] |
| 6 (direct attachment of Y—Z) | 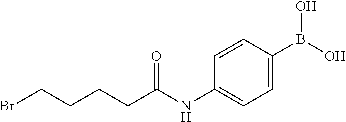 [SMILES: O=C(CCCCBr)Nc1ccc(B(O)O)cc1] | 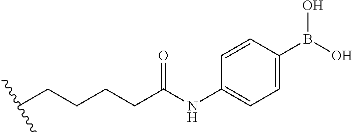 [SMILES: *CCCCC(=O)Nc1ccc(B(O)O)cc1] |
| 7 (functionalization of No. 27 in Table A) | 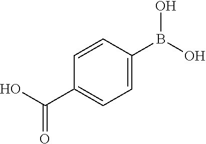 [SMILES: O=C(O)c1ccc(B(O)O)cc1] | 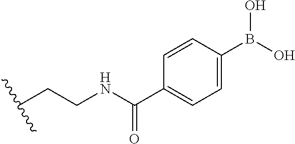 [SMILES: *CCNC(=O)c1ccc(B(O)O)cc1] |
| 8 (functionalization of No. 27 in Table A) | 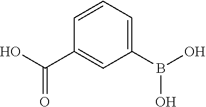 [SMILES: O=C(O)c1cccc(B(O)O)c1] | 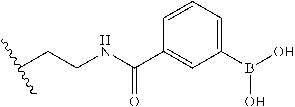 [SMILES: *CCNC(=O)c1cccc(B(O)O)c1] |
| 9 (functionalization of No. 32 in Table A) | 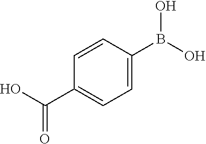 [SMILES: O=C(O)c1ccc(B(O)O)cc1] | 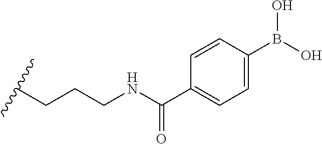 [SMILES: *CCCNC(=O)c1ccc(B(O)O)cc1] |
| 10 (functionalization of No. 32 in Table A) | 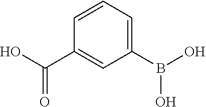 [SMILES: O=C(O)c1cccc(B(O)O)c1] | 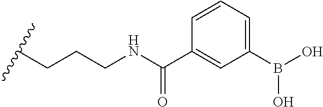 [SMILES: *CCCNC(=O)c1cccc(B(O)O)c1] |
| 11 (functionalization of No. 33 in Table A) | 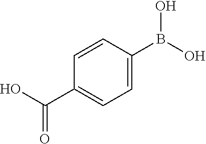 [SMILES: O=C(O)c1ccc(B(O)O)cc1] | 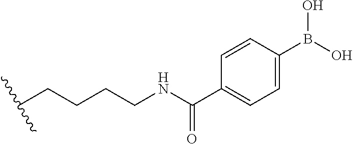 [SMILES: *CCCCNC(=O)c1ccc(B(O)O)cc1] |
| 12 (functionalization of No. 33 in Table A) | 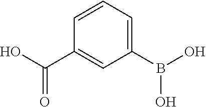 [SMILES: O=C(O)c1cccc(B(O)O)c1] | 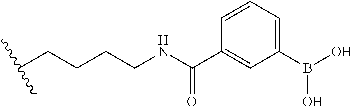 [SMILES: *CCCCNC(=O)c1cccc(B(O)O)c1] |

The synthetic route in Scheme Xb illustrates a general method for preparing benzodiazepine-connector 2 derivatives. The method involves attaching the desired substituents to the carbonyl substituent. The desired R group attached at the carbonyl substituent can be installed by reacting carboxylic acid 4 with common coupling reagents such as 1-ethyl-3-(3-dimethylaminopropyl)carbodiimide (EDC) and hydroxybenzotriazole (HOBt) and then further reacting the activated ester 6 with the appropriate nucleophile, for example, amine 7, to provide 8a (benzodiazepine-connector 2 derivative). For example, Scheme Xb provides for a connector Y (e.g. Y₁, Y₂, Y₃ or Y₄) wherein Y is —NH—R (e.g., —NH—R of 8a).

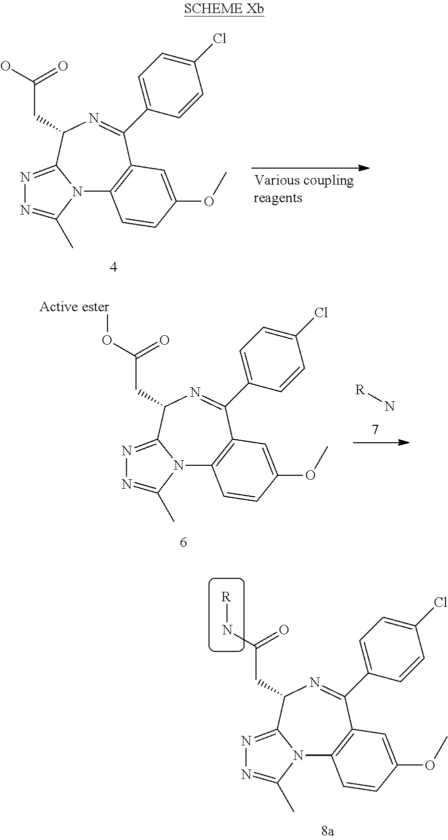

For example, R may be selected from the group consisting of:

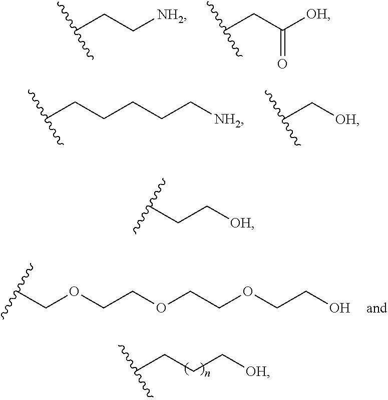

where n may be 0, 1, 2, 3, 4 or 5.

In some embodiments, R may generally be represented for example, by:

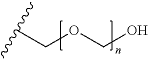

where n may be 0, 1, 2, 3, 4, 5, or 6.

Additional examples for 7 and —NH—R (e.g., Y) can be found in Table B, seen below:

TABLE B

| | | —NH—R |
|---|---|---|
| No. | 7 | (e.g., —Y) |
| 1 | 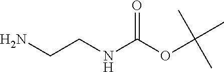 | 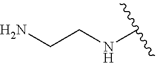 [SMILES: *NCCN] |
| 2 | 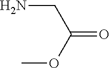 | 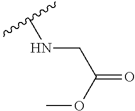 [SMILES: *NCC(=O)OC] |
| 3 | 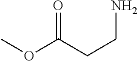 | 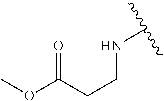 [SMILES: *NCCC(=O)OC] |
| 4 | 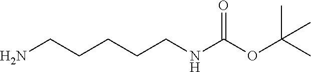 | 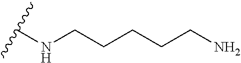 [SMILES: *NCCCCCN] |
| 5 | 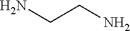 | 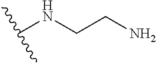 [SMILES: *NCCN] |
| 6 | 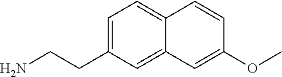 | 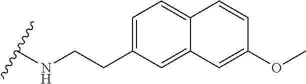 [SMILES: *NCCc1ccc2ccc(OC)cc2c1] |
| 7 | 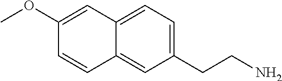 | 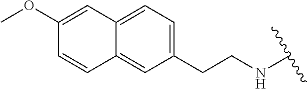 [SMILES: *NCCc1ccc2cc(OC)ccc2c1] |
| 8 | 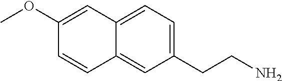 | 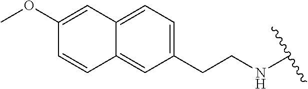 [SMILES: *NCCc1ccc2cc(OC)ccc2c1] |
| 9 | 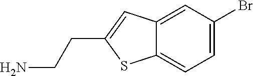 [SMILES: NCCc1cc2cc(Br)ccc2s1] | 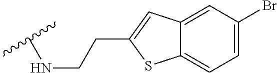 [SMILES: *NCCc1cc2cc(Br)ccc2s1] |
| 10 | 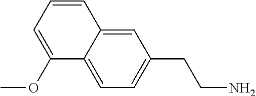 | 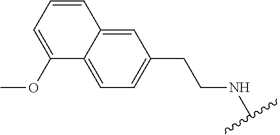 [SMILES: *NCCc1ccc2c(OC)cccc2c1] |
| 11 | 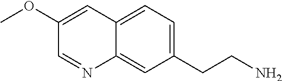 | 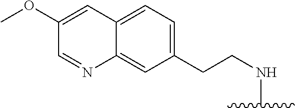 [SMILES: *NCCc1ccc2cc(OC)cnc2c1] |
| 12 | 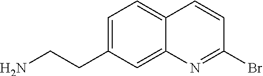 [SMILES: NCCc1ccc2ccc(Br)nc2c1] | 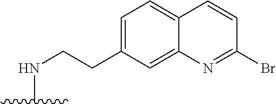 [SMILES: *NCCc1ccc2ccc(Br)nc2c1] |
| 13 | 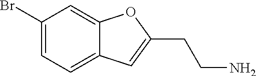 [SMILES: NCCc1cc2ccc(Br)cc2o1] | 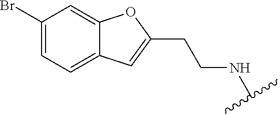 [SMILES: *NCCc1cc2ccc(Br)cc2o1] |
| 14 | 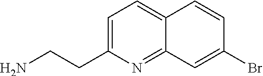 [SMILES: NCCc1ccc2ccc(Br)cc2n1] | 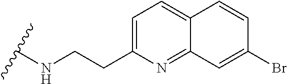 [SMILES: *NCCc1ccc2ccc(Br)cc2n1] |
| 15 | 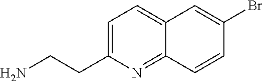 [SMILES: NCCc1ccc2cc(Br)ccc2n1] | 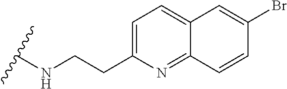 [SMILES: *NCCc1ccc2cc(Br)ccc2n1] |
| 16 | 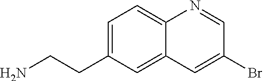 [SMILES: NCCc1ccc2ncc(Br)cc2c1] | 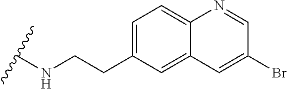 [SMILES: *NCCc1ccc2ncc(Br)cc2c1] |
| 17 | 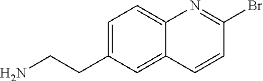 [SMILES: NCCc1ccc2nc(Br)ccc2c1] | 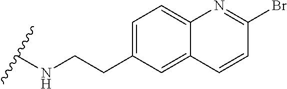 [SMILES: *NCCc1ccc2nc(Br)ccc2c1] |
| 18 | 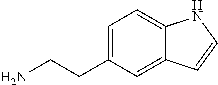 [SMILES: NCCc1ccc2[nH]ccc2c1] | 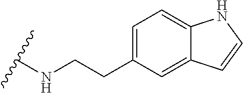 [SMILES: *NCCc1ccc2[nH]ccc2c1] |
| 19 | 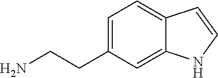 [SMILES: NCCc1ccc2cc[nH]c2c1] | 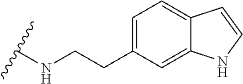 [SMILES: *NCCc1ccc2cc[nH]c2c1] |
| 20 | 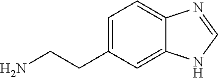 [SMILES: NCCc1ccc2nc[nH]c2c1] | 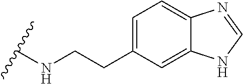 [SMILES: *NCCc1ccc2nc[nH]c2c1] |
| 21 | 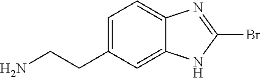 [SMILES: NCCc1ccc2nc(Br)[nH]c2c1] | 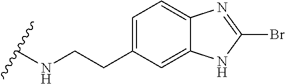 [SMILES: *NCCc1ccc2nc(Br)[nH]c2c1] |
| 22 | 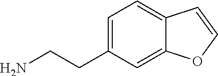 [SMILES: NCCc1ccc2ccoc2c1] | 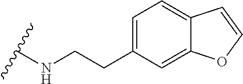 [SMILES: *NCCc1ccc2ccoc2c1] |
| 23 | 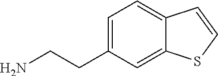 [SMILES: NCCc1ccc2ccsc2c1] | 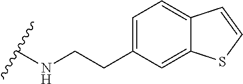 [SMILES: *NCCc1ccc2ccsc2c1] |
| 24 | 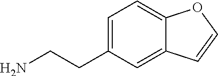 [SMILES: NCCc1ccc2occc2c1] | 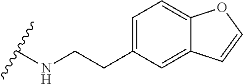 [SMILES: *NCCc1ccc2occc2c1] |
| 25 | 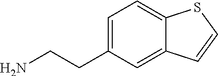 [SMILES: NCCc1ccc2sccc2c1] | 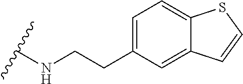 [SMILES: *NCCc1ccc2sccc2c1] |
| 26 | 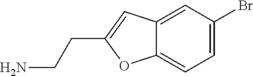 [SMILES: NCCc1cc2cc(Br)ccc2o1] | 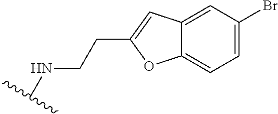 [SMILES: *NCCc1cc2cc(Br)ccc2o1] |
| 27 | 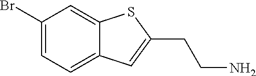 [SMILES: NCCc1cc2ccc(Br)cc2s1] | 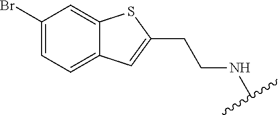 [SMILES: *NCCc1cc2ccc(Br)cc2s1] |
| 28 | 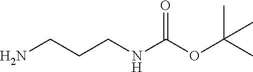 | 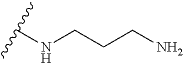 [SMILES: *NCCCN] |
| 29 | 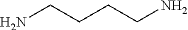 | 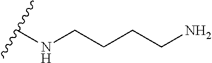 [SMILES: *NCCCCN] |

The following table (Table V) contains exemplary benzodiazepine-connector 2 derivatives (e.g., 8a of Scheme Xb) that include a ligand moiety (X) and a connector (Y). A person of skill in the art would understand that such derivatives can be modified to include a disclosed pharmacophore Z.

TABLE V

| No. | Compound Structure |
|---|---|
| 1 | 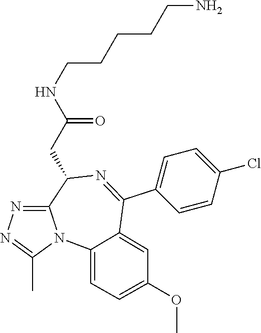 [SMILES: COc1ccc2c(c1)C(c1ccc(Cl)cc1)=N[C@@H](CC(=O)NCCCCCN)c1nnc(C)n1-2] |
| 2 | 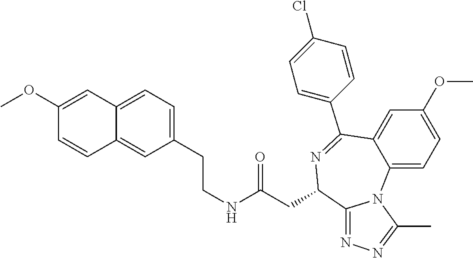 |
| 3 | 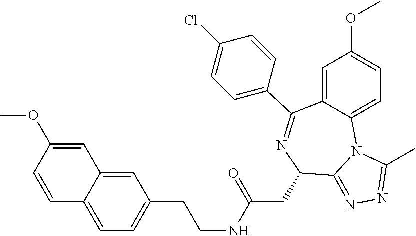 |
| 4 | 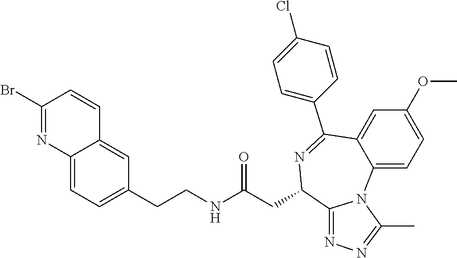 [SMILES: COc1ccc2c(c1)C(c1ccc(Cl)cc1)=N[C@@H](CC(=O)NCCc1ccc3nc(Br)ccc3c1)c1nnc(C)n1-2] |
| 5 | 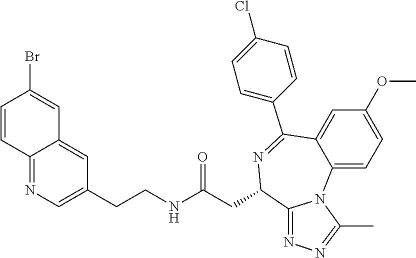 [SMILES: COc1ccc2c(c1)C(c1ccc(Cl)cc1)=N[C@@H](CC(=O)NCCc1cnc3ccc(Br)cc3c1)c1nnc(C)n1-2] |
| 6 | 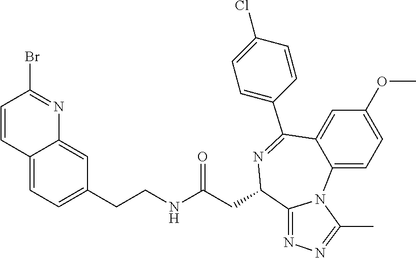 [SMILES: COc1ccc2c(c1)C(c1ccc(Cl)cc1)=N[C@@H](CC(=O)NCCc1ccc3ccc(Br)nc3c1)c1nnc(C)n1-2] |
| 7 | 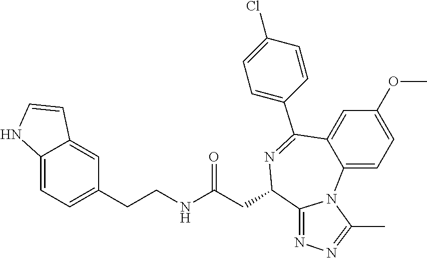 [SMILES: COc1ccc2c(c1)C(c1ccc(Cl)cc1)=N[C@@H](CC(=O)NCCc1ccc3[nH]ccc3c1)c1nnc(C)n1-2] |
| 8 | 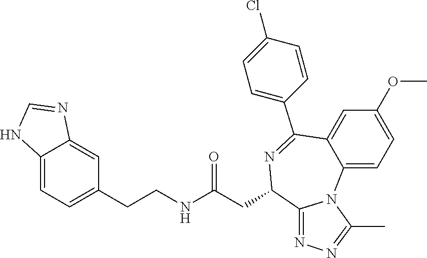 [SMILES: COc1ccc2c(c1)C(c1ccc(Cl)cc1)=N[C@@H](CC(=O)NCCc1ccc3[nH]cnc3c1)c1nnc(C)n1-2] |
| 9 | 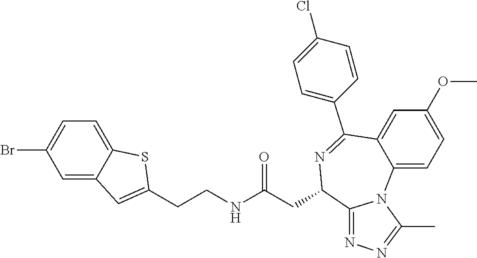 [SMILES: COc1ccc2c(c1)C(c1ccc(Cl)cc1)=N[C@@H](CC(=O)NCCc1cc3cc(Br)ccc3s1)c1nnc(C)n1-2] |
| 10 | 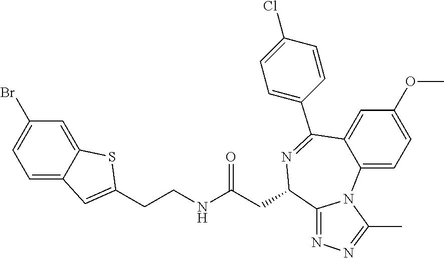 [SMILES: COc1ccc2c(c1)C(c1ccc(Cl)cc1)=N[C@@H](CC(=O)NCCc1cc3ccc(Br)cc3s1)c1nnc(C)n1-2] |
| 11 | 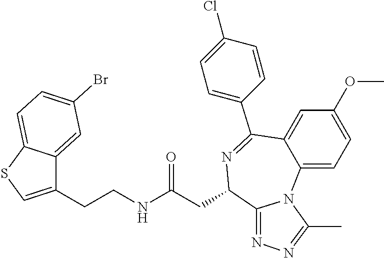 [SMILES: COc1ccc2c(c1)C(c1ccc(Cl)cc1)=N[C@@H](CC(=O)NCCc1csc3ccc(Br)cc13)c1nnc(C)n1-2] |
| 12 | 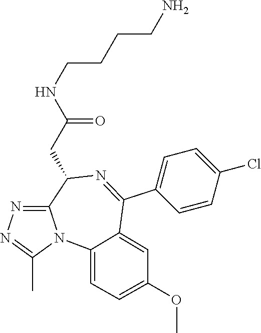 [SMILES: COc1ccc2c(c1)C(c1ccc(Cl)cc1)=N[C@@H](CC(=O)NCCCCN)c1nnc(C)n1-2] |
| 13 | 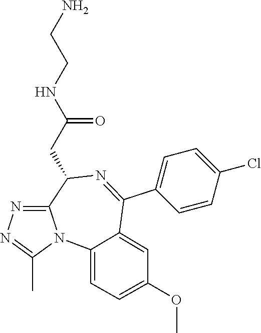 [SMILES: COc1ccc2c(c1)C(c1ccc(Cl)cc1)=N[C@@H](CC(=O)NCCN)c1nnc(C)n1-2] |
| 14 | 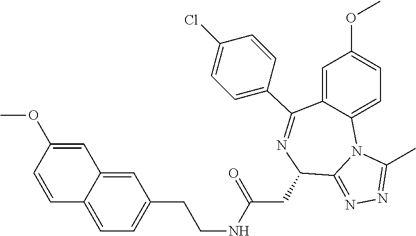 |
| 15 | 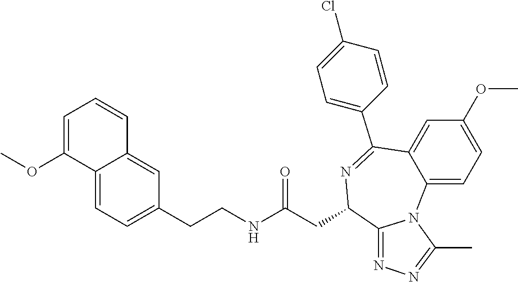 |
| 16 | 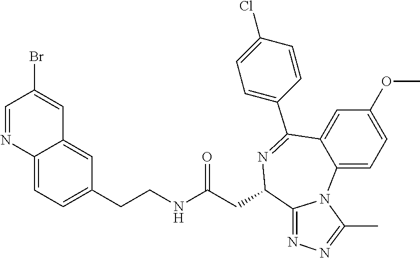 [SMILES: COc1ccc2c(c1)C(c1ccc(Cl)cc1)=N[C@@H](CC(=O)NCCc1ccc3ncc(Br)cc3c1)c1nnc(C)n1-2] |
| 17 | 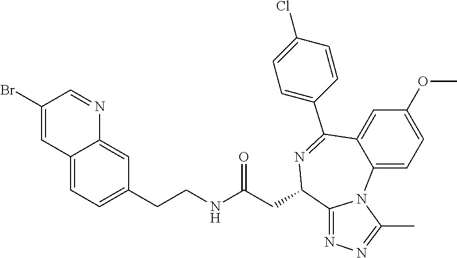 [SMILES: COc1ccc2c(c1)C(c1ccc(Cl)cc1)=N[C@@H](CC(=O)NCCc1ccc3cc(Br)cnc3c1)c1nnc(C)n1-2] |
| 18 | 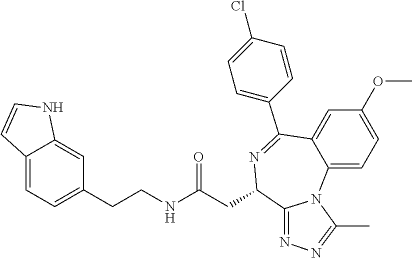 [SMILES: COc1ccc2c(c1)C(c1ccc(Cl)cc1)=N[C@@H](CC(=O)NCCc1ccc3cc[nH]c3c1)c1nnc(C)n1-2] |
| 19 | 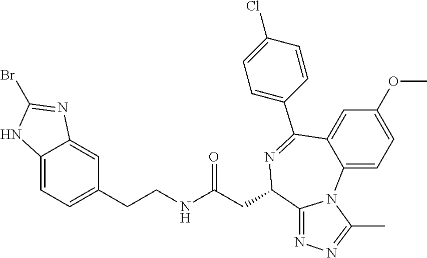 [SMILES: COc1ccc2c(c1)C(c1ccc(Cl)cc1)=N[C@@H](CC(=O)NCCc1ccc3[nH]c(Br)nc3c1)c1nnc(C)n1-2] |
| 20 | 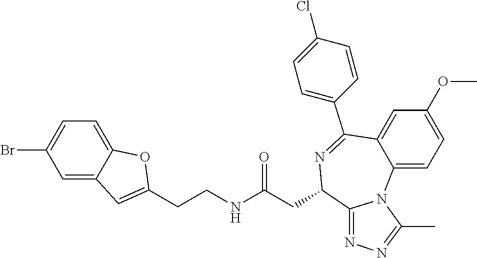 [SMILES: COc1ccc2c(c1)C(c1ccc(Cl)cc1)=N[C@@H](CC(=O)NCCc1cc3cc(Br)ccc3o1)c1nnc(C)n1-2] |
| 21 | 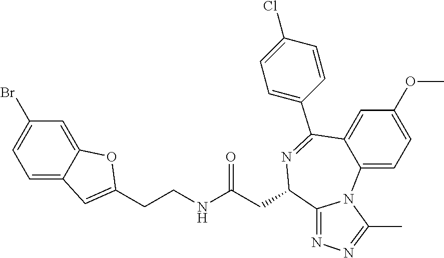 [SMILES: COc1ccc2c(c1)C(c1ccc(Cl)cc1)=N[C@@H](CC(=O)NCCc1cc3ccc(Br)cc3o1)c1nnc(C)n1-2] |
| 22 | 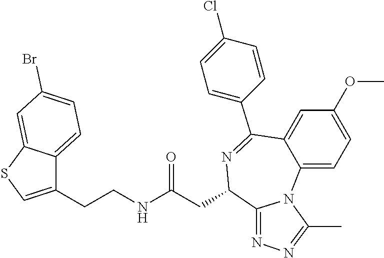 [SMILES: COc1ccc2c(c1)C(c1ccc(Cl)cc1)=N[C@@H](CC(=O)NCCc1csc3cc(Br)ccc13)c1nnc(C)n1-2] |
| 23 | 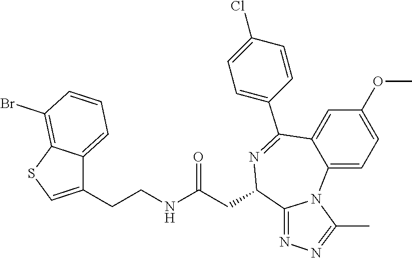 [SMILES: COc1ccc2c(c1)C(c1ccc(Cl)cc1)=N[C@@H](CC(=O)NCCc1csc3c(Br)cccc13)c1nnc(C)n1-2] |
| 24 | 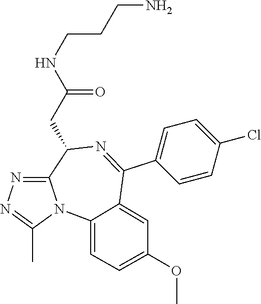 [SMILES: COc1ccc2c(c1)C(c1ccc(Cl)cc1)=N[C@@H](CC(=O)NCCCN)c1nnc(C)n1-2] |

Any free amino group seen in the —NH—R examples (e.g., Y examples) of Table B above may be functionalized further to include additional functional groups, e.g., a benzoyl moiety.

In another embodiment, the attachment point identified in B may be further elaborated to incorporate not only a connector moiety, but also a linker, as e.g., represented by:

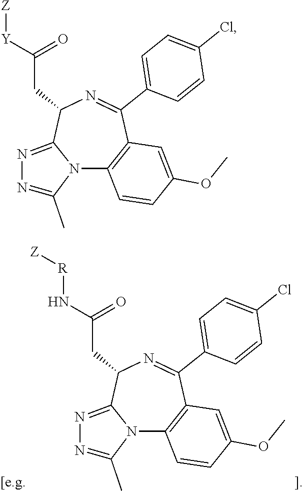

The Y—Z moiety may be formed from direct attachment of Y—Z to the carbonyl, or the Y—Z moiety may be formed from the further functionalization of any free amino group seen in the —NH—R examples (i.e., Y examples) of Table B above to include the linker moiety (Z). Examples of —NH—R—Z groups (e.g., Y—Z groups) having a boronic acid, diol or silanol linker (Z) can be found in Table B″, seen below. It is clear from the linker section described above that a first monomer that has a boronic acid linker may be capable of forming a multimer with a second monomer that has a diol linker. In another embodiment, a first monomer that has a silanol linker may be capable of forming a multimer with a second monomer that has the same or different silanol linker

TABLE B″

| | | —NH—R—Z |
|---|---|---|
| No. | Reagent | (e.g., —Y—Z) |
| 1 (functionalization of No. 3 in Table B) | 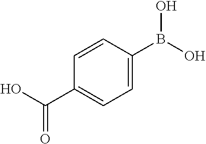 [SMILES: O=C(O)c1ccc(B(O)O)cc1] | 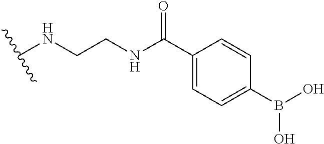 [SMILES: *NCCNC(=O)c1ccc(B(O)O)cc1] |
| 2 (functionalization of No. 3 in Table B) | 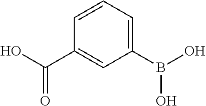 [SMILES: O=C(O)c1cccc(B(O)O)c1] | 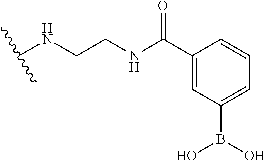 [SMILES: *NCCNC(=O)c1cccc(B(O)O)c1] |
| 3 (functionalization of No. 3 in Table B) | 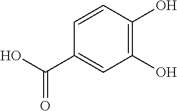 [SMILES: O=C(O)c1ccc(O)c(O)c1] | 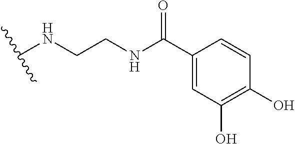 [SMILES: *NCCNC(=O)c1ccc(O)c(O)c1] |
| 4 (functionalization of No. 3 in Table B) | 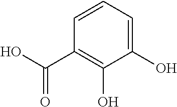 [SMILES: O=C(O)c1cccc(O)c1O] | 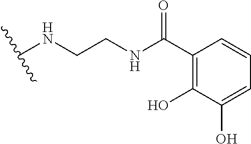 [SMILES: *NCCNC(=O)c1cccc(O)c1O] |
| 5 (functionalization of No. 3 in Table B) | 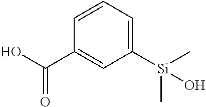 | 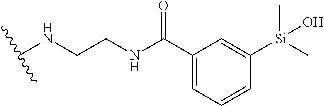 [SMILES: *NCCNC(=O)c1cccc([Si](C)(C)O)c1] |
| 6 (functionalization of No. 3 in Table B) | 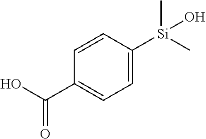 | 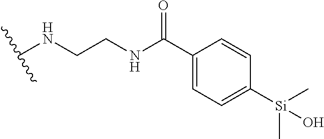 [SMILES: *NCCNC(=O)c1ccc([Si](C)(C)O)cc1] |
| 7 (functionalization of No. 55 in Table B) | 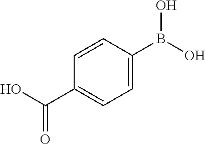 [SMILES: O=C(O)c1ccc(B(O)O)cc1] | 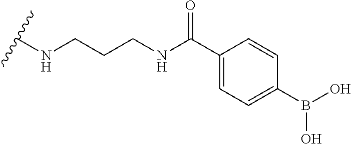 [SMILES: *NCCCNC(=O)c1ccc(B(O)O)cc1] |
| 8 (functionalization of No. 55 in Table B) | 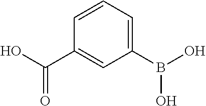 [SMILES: O=C(O)c1cccc(B(O)O)c1] | 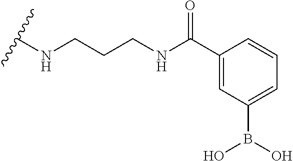 [SMILES: *NCCCNC(=O)c1cccc(B(O)O)c1] |
| 9 (functionalization of No. 55 in Table B) | 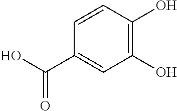 [SMILES: O=C(O)c1ccc(O)c(O)c1] | 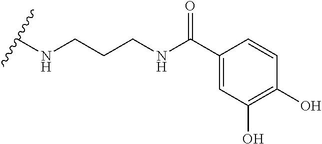 [SMILES: *NCCCNC(=O)c1ccc(O)c(O)c1] |
| 10 (functionalization of No. 55 in Table B) | 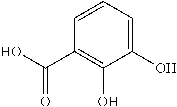 [SMILES: O=C(O)c1cccc(O)c1O] | 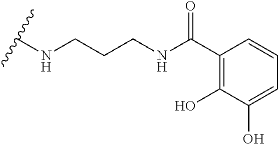 [SMILES: *NCCCNC(=O)c1cccc(O)c1O] |
| 11 (functionalization of No. 55 in Table B) | 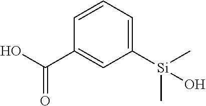 | 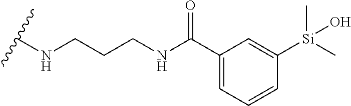 [SMILES: *NCCCNC(=O)c1cccc([Si](C)(C)O)c1] |
| 12 (functionalization of No. 55 in Table B) | 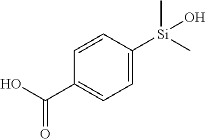 | 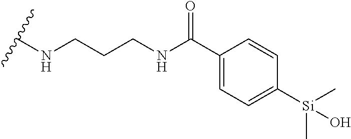 [SMILES: *NCCCNC(=O)c1ccc([Si](C)(C)O)cc1] |
| 13 (functionalization of No. 56 in Table B) | 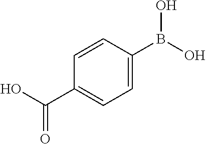 [SMILES: O=C(O)c1ccc(B(O)O)cc1] | 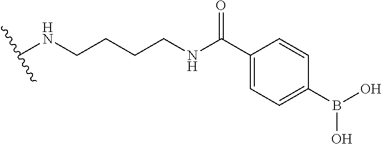 [SMILES: *NCCCCNC(=O)c1ccc(B(O)O)cc1] |
| 14 (functionalization of No. 56 in Table B) | 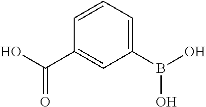 [SMILES: O=C(O)c1cccc(B(O)O)c1] | 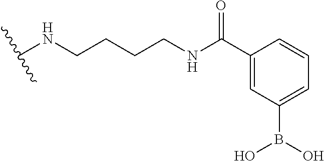 [SMILES: *NCCCCNC(=O)c1cccc(B(O)O)c1] |
| 15 (functionalization of No. 56 in Table B) | 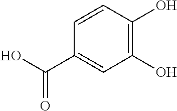 [SMILES: O=C(O)c1ccc(O)c(O)c1] | 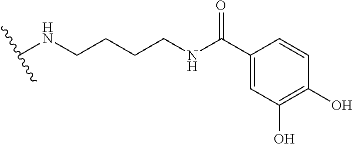 [SMILES: *NCCCCNC(=O)c1ccc(O)c(O)c1] |
| 16 (functionalization of No. 56 in Table B) | 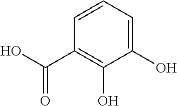 [SMILES: O=C(O)c1cccc(O)c1O] | 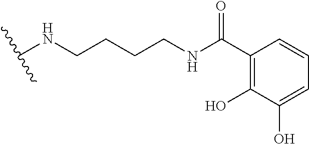 [SMILES: *NCCCCNC(=O)c1cccc(O)c1O] |
| 17 (functionalization of No. 56 in Table B) | 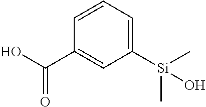 | 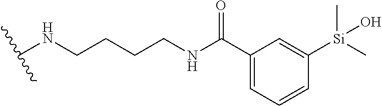 [SMILES: *NCCCCNC(=O)c1cccc([Si](C)(C)O)c1] |
| 18 (functionalization of No. 56 in Table B) | 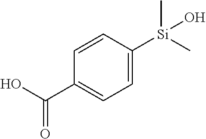 | 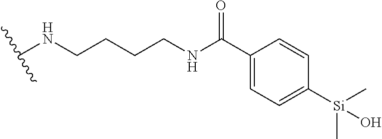 [SMILES: *NCCCCNC(=O)c1ccc([Si](C)(C)O)cc1] |
| 19 (functionalization of No. 14 in Table B) | 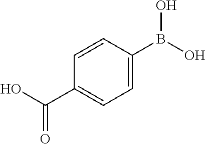 [SMILES: O=C(O)c1ccc(B(O)O)cc1] | 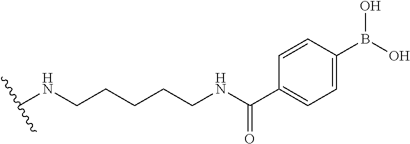 [SMILES: *NCCCCCNC(=O)c1ccc(B(O)O)cc1] |
| 20 (functionalization of No. 14 in Table B) | 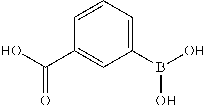 [SMILES: O=C(O)c1cccc(B(O)O)c1] | 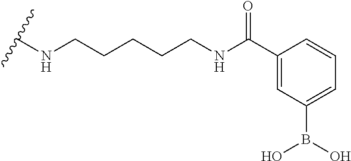 [SMILES: *NCCCCCNC(=O)c1cccc(B(O)O)c1] |
| 21 (functionalization of No. 14 in Table B) | 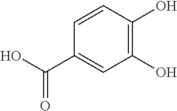 [SMILES: O=C(O)c1ccc(O)c(O)c1] | 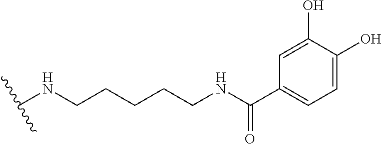 [SMILES: *NCCCCCNC(=O)c1ccc(O)c(O)c1] |
| 22 (functionalization of No. 14 in Table B) | 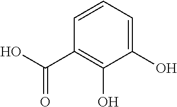 | 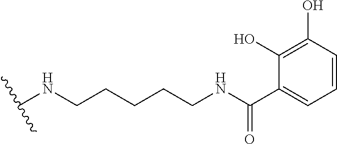 [SMILES: *NCCCCCNC(=O)c1cccc(O)c1O] |
| 23 (functionalization of No. 14 in Table B) | 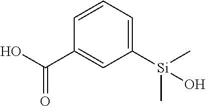 | 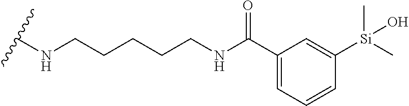 [SMILES: *NCCCCCNC(=O)c1cccc([Si](C)(C)O)c1] |
| 24 (functionalization of No. 14 in Table B) | 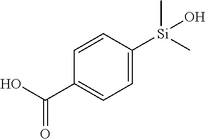 | 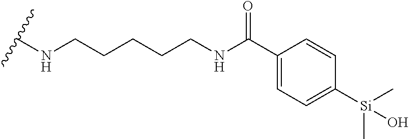 [SMILES: *NCCCCCNC(=O)c1ccc([Si](C)(C)O)cc1] |

In another embodiment, the two attachment points identified in A and B may be further elaborated to incorporate not only a connector moiety, but also a linker.

Scheme Xc provides a synthetic procedure for making A derivatives having various connectors attached to both the benzodiazepine compound and to any of the above-identified linkers (Z¹, Z², Z³ and Z⁴). In the scheme below, the linker moiety is designated by Z. Phenol 1 is converted to carboxylic acid 10 using ethyl-2-bromoacetate, followed by hydrolysis. Following formation of 10, the general procedure outlined in Scheme Xb can be utilized in the synthesis of the benzodiazepine-connector 1 derivative 12. For example, Scheme Xc provides for a connector Y (e.g. Y₁, Y₂, Y₃ or Y₄) attached to a linker moiety (Z), wherein Y is —CH₂—C(O)—R— (e.g., —CH₂—C(O)—R— of 12).

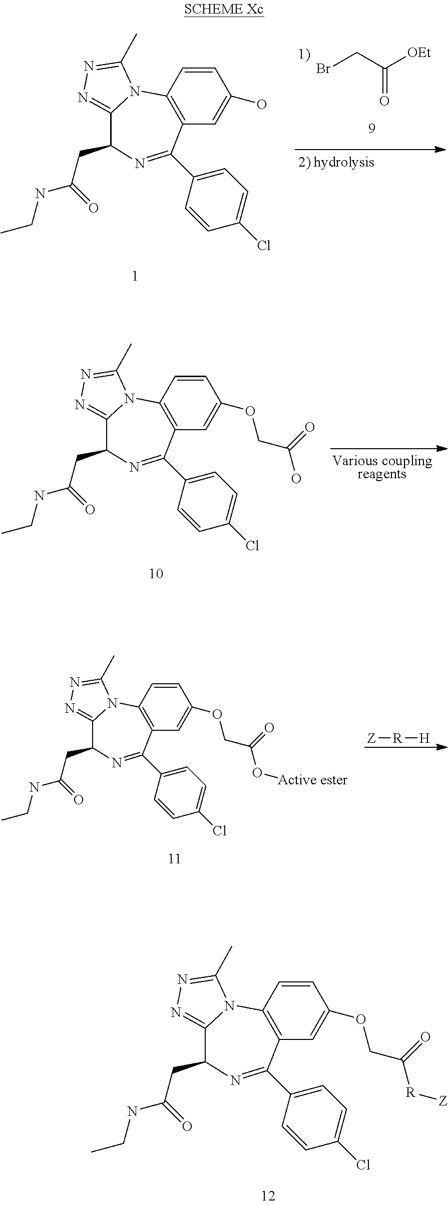

For example, R—Z may be selected from the group consisting of:

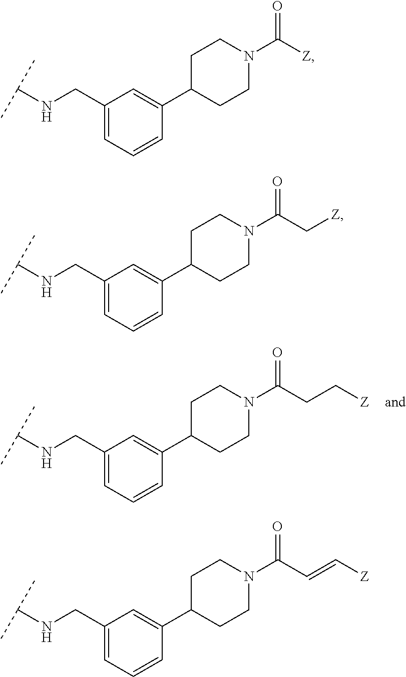

Scheme Xd provides an exemplary synthetic procedure for making B derivatives having various connectors attached to both the benzodiazepine compound and to any of the above-identified linkers (Z¹, Z², Z³ and Z⁴). In the scheme below, the linker moiety is designated by Z. Activated ester 6 is reacted with various nucleophiles to provide benzodiazepine-connector 2 derivative 8b. For example, Scheme Xd provides for a connector Y (e.g. Y₁, Y₂, Y₃ or Y₄) attached to a linker moiety (Z), wherein Y is —R— (e.g., —R— of 8b).

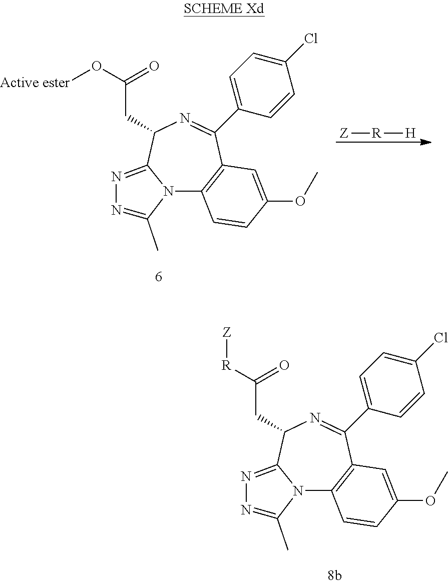

For example, R—Z (i.e., Y—Z) may be selected from the group consisting of:

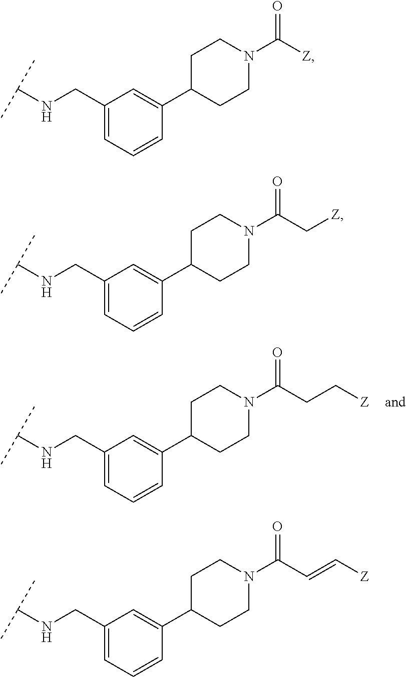

Additional examples for Z—R—H and —R—Z that can be utilized in Scheme Xc and Scheme Xd can be found in Table C, seen below:

TABLE C

| Example | |
|---|---|
| No. | Z—R—H |
| 1 | 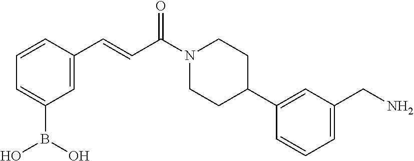 |
| 2 | 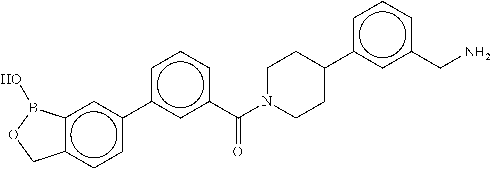 |
| 3 | 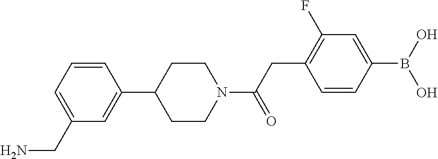 |
| 4 | 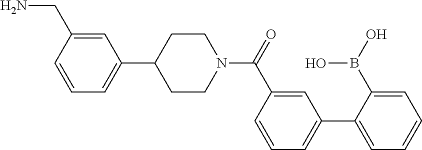 |
| 5 | 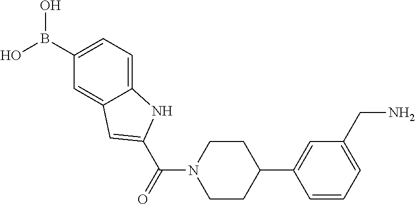 |
| 6 | 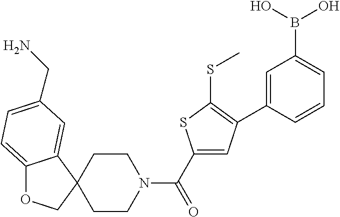 |
| 7 | 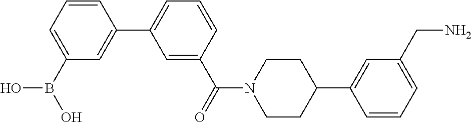 |
| 8 | 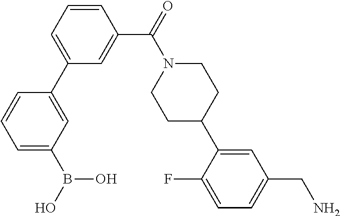 |
| 9 | 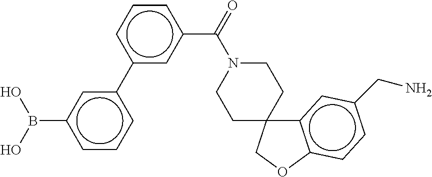 |
| 10 | 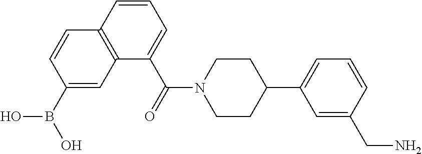 |
| 11 | 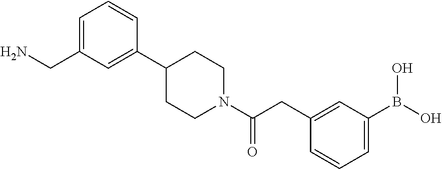 |
| 12 | 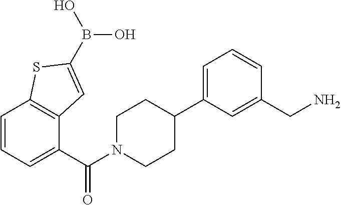 |
| 13 | 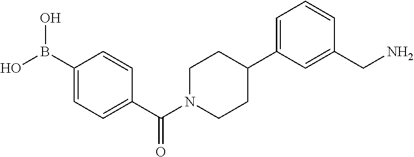 |
| 14 | 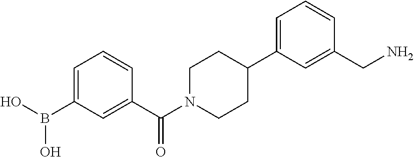 |
| 15 | 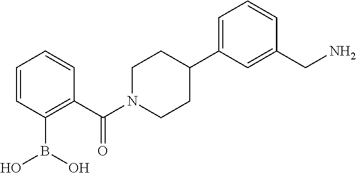 |
| 16 | 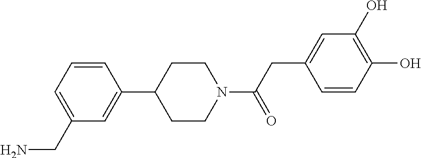 |
| 17 | 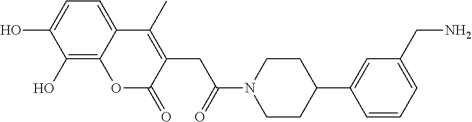 |
| 18 | 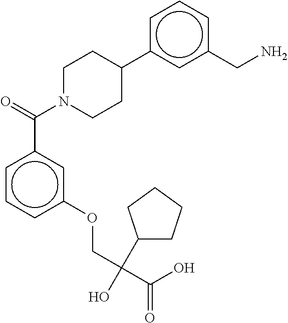 |
| 19 | 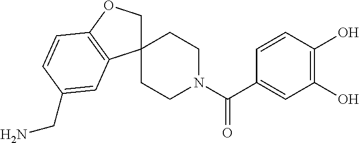 |
| 20 | 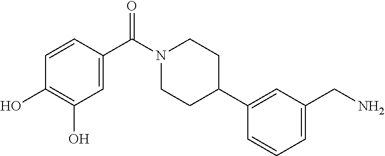 |
| 21 | 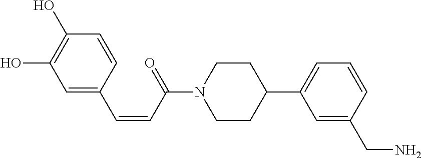 |
| 22 | 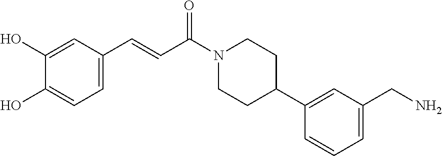 |
| 23 | 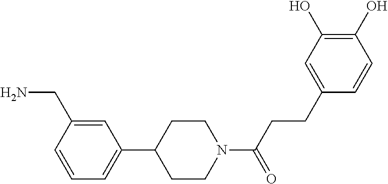 |
| 24 | 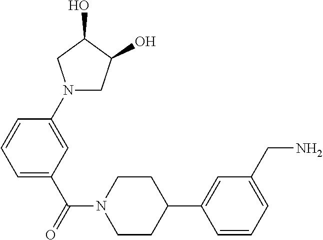 |
| 25 | 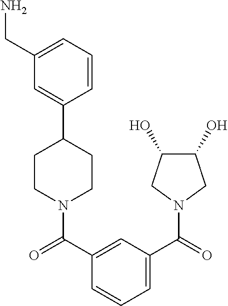 |
| 26 | 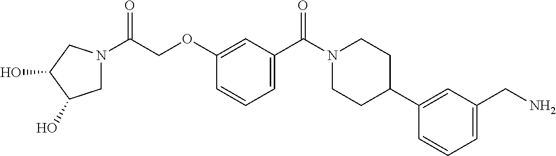 |
| 27 | 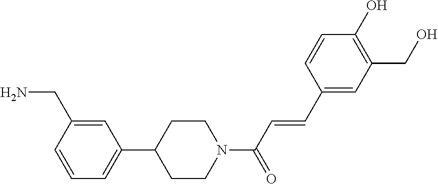 |
| 28 | 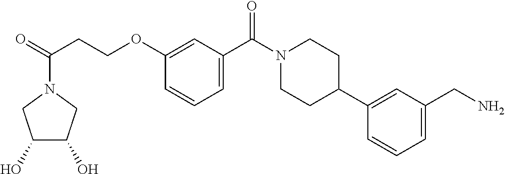 |
| 29 | 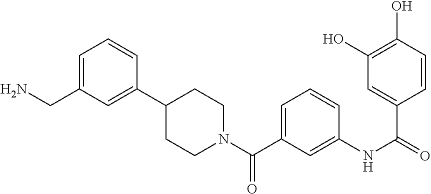 |
| 30 | 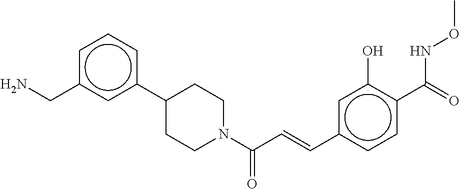 |
| 31 | 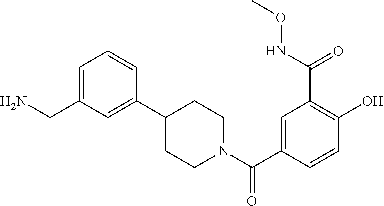 |
| 32 | 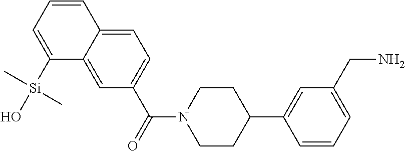 |
| 33 | 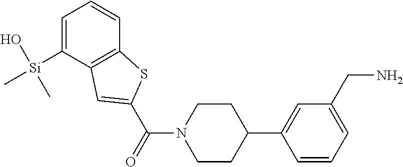 |
| 34 | 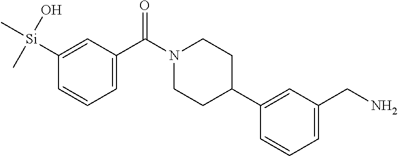 |
| 35 | 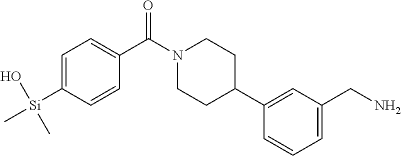 |
| 36 | 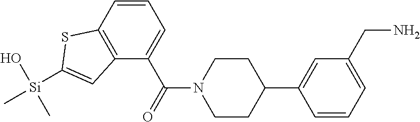 |
| 37 | 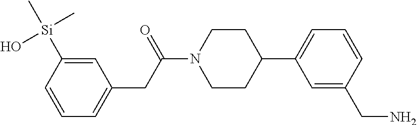 |
| 38 | 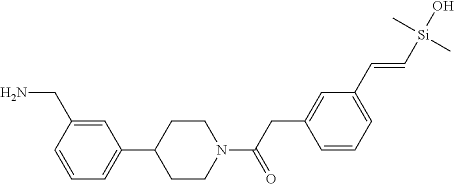 |
| 39 | 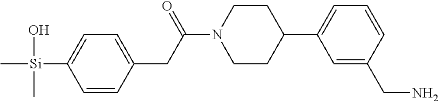 |
| Example | |
| No. | —R—Z |
| 1 | 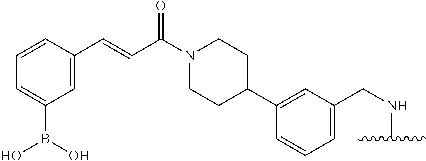 [SMILES: *NCc1cccc(C2CCN(C(=O)/C=C/c3cccc(B(O)O)c3)CC2)c1] |
| 2 | 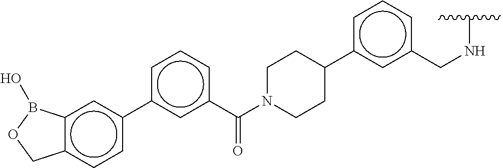 |
| 3 | 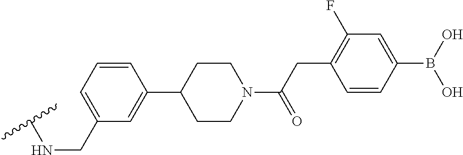 [SMILES: *NCc1cccc(C2CCN(C(=O)Cc3ccc(B(O)O)cc3F)CC2)c1] |
| 4 | 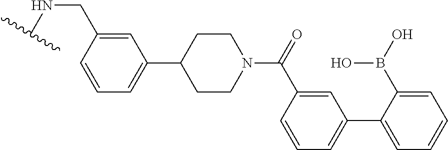 [SMILES: *NCc1cccc(C2CCN(C(=O)c3cccc(-c4ccccc4B(O)O)c3)CC2)c1] |
| 5 | 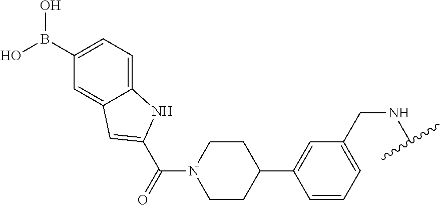 [SMILES: *NCc1cccc(C2CCN(C(=O)c3cc4cc(B(O)O)ccc4[nH]3)CC2)c1] |
| 6 | 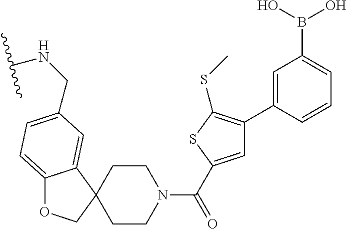 [SMILES: *NCc1ccc2c(c1)C1(CCN(C(=O)c3cc(-c4cccc(B(O)O)c4)c(SC)s3)CC1)CO2] |
| 7 | 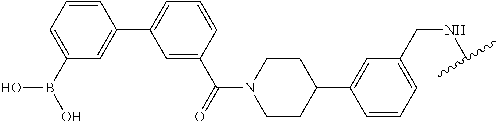 [SMILES: *NCc1cccc(C2CCN(C(=O)c3cccc(-c4cccc(B(O)O)c4)c3)CC2)c1] |
| 8 | 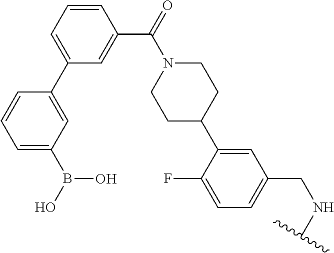 [SMILES: *NCc1ccc(F)c(C2CCN(C(=O)c3cccc(-c4cccc(B(O)O)c4)c3)CC2)c1] |
| 9 | 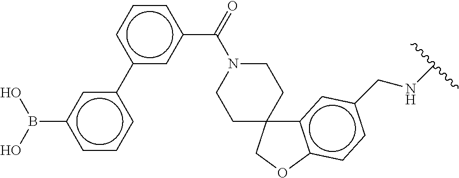 |
| 10 | 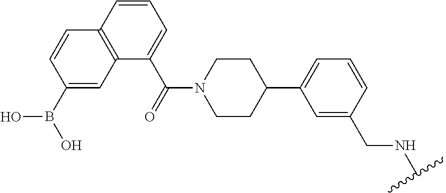 [SMILES: *NCc1cccc(C2CCN(C(=O)c3cccc4ccc(B(O)O)cc34)CC2)c1] |
| 11 | 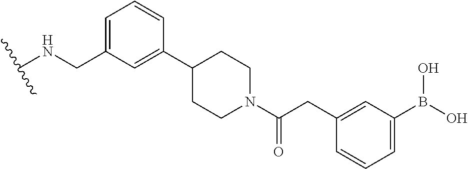 [SMILES: *NCc1cccc(C2CCN(C(=O)Cc3cccc(B(O)O)c3)CC2)c1] |
| 12 | 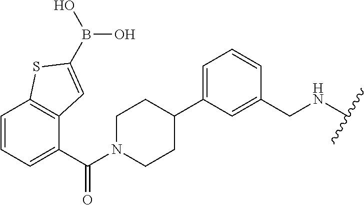 [SMILES: *NCc1cccc(C2CCN(C(=O)c3cccc4sc(B(O)O)cc34)CC2)c1] |
| 13 | 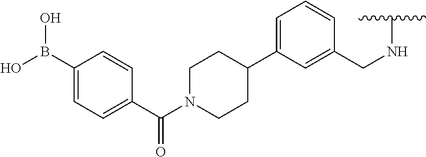 [SMILES: *NCc1cccc(C2CCN(C(=O)c3ccc(B(O)O)cc3)CC2)c1] |
| 14 | 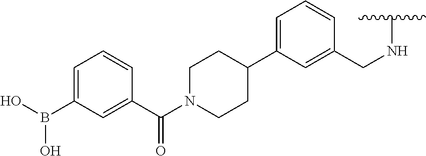 [SMILES: *NCc1cccc(C2CCN(C(=O)c3cccc(B(O)O)c3)CC2)c1] |
| 15 | 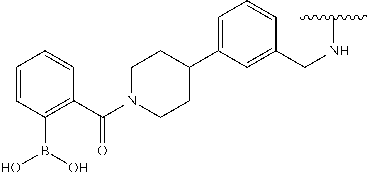 [SMILES: *NCc1cccc(C2CCN(C(=O)c3ccccc3B(O)O)CC2)c1] |
| 16 | 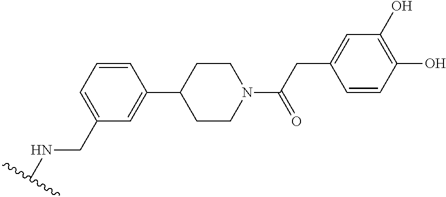 [SMILES: *NCc1cccc(C2CCN(C(=O)Cc3ccc(O)c(O)c3)CC2)c1] |
| 17 | 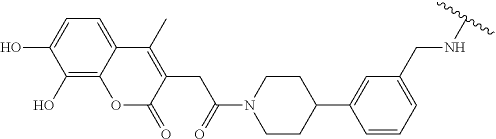 [SMILES: *NCc1cccc(C2CCN(C(=O)Cc3c(C)c4ccc(O)c(O)c4oc3=O)CC2)c1] |
| 18 | 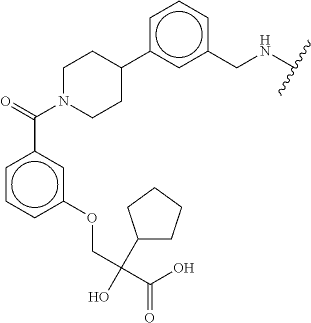 |
| 19 | 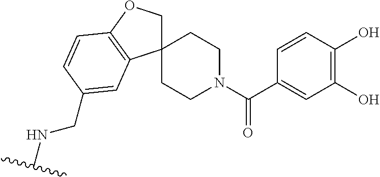 [SMILES: *NCc1ccc2c(c1)C1(CCN(C(=O)c3ccc(O)c(O)c3)CC1)CO2] |
| 20 | 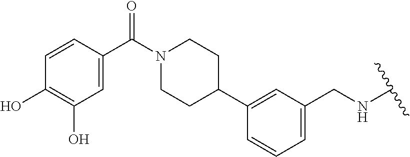 [SMILES: *NCc1cccc(C2CCN(C(=O)c3ccc(O)c(O)c3)CC2)c1] |
| 21 | 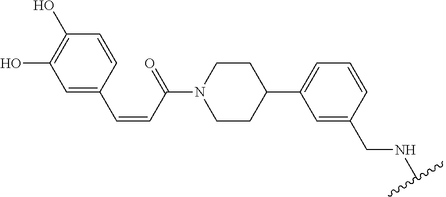 [SMILES: *NCc1cccc(C2CCN(C(=O)/C=C\c3ccc(O)c(O)c3)CC2)c1] |
| 22 | 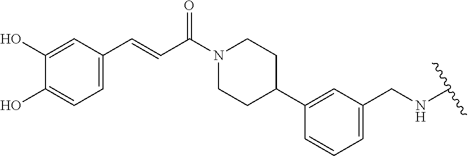 [SMILES: *NCc1cccc(C2CCN(C(=O)/C=C/c3ccc(O)c(O)c3)CC2)c1] |
| 23 | 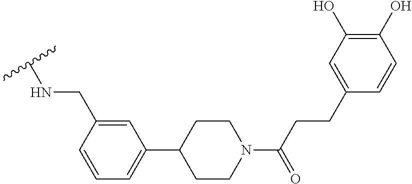 [SMILES: *NCc1cccc(C2CCN(C(=O)CCc3ccc(O)c(O)c3)CC2)c1] |
| 24 | 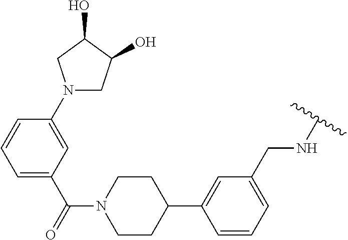 [SMILES: *NCc1cccc(C2CCN(C(=O)c3cccc(N4C[C@@H](O)[C@@H](O)C4)c3)CC2)c1] |
| 25 | 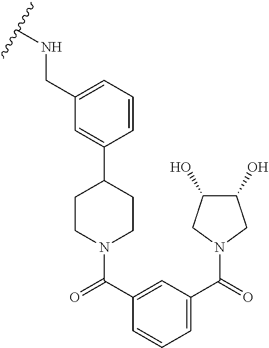 [SMILES: *NCc1cccc(C2CCN(C(=O)c3cccc(C(=O)N4C[C@@H](O)[C@@H](O)C4)c3)CC2)c1] |
| 26 | 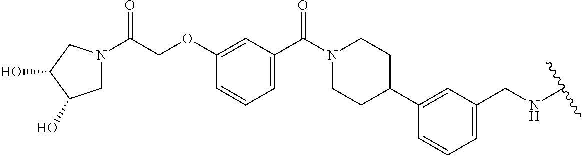 [SMILES: *NCc1cccc(C2CCN(C(=O)c3cccc(OCC(=O)N4C[C@@H](O)[C@@H](O)C4)c3)CC2)c1] |
| 27 | 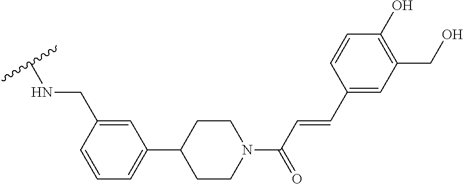 [SMILES: *NCc1cccc(C2CCN(C(=O)/C=C/c3ccc(O)c(CO)c3)CC2)c1] |
| 28 | 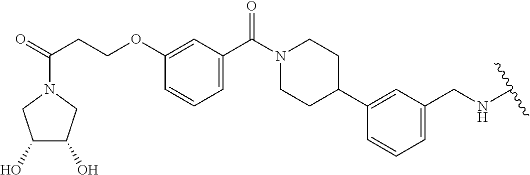 [SMILES: *NCc1cccc(C2CCN(C(=O)c3cccc(OCCC(=O)N4C[C@@H](O)[C@@H](O)C4)c3)CC2)c1] |
| 29 | 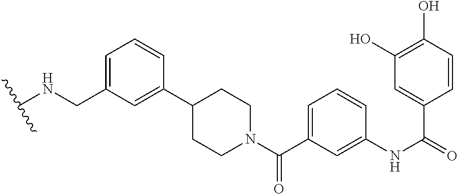 [SMILES: *NCc1cccc(C2CCN(C(=O)c3cccc(NC(=O)c4ccc(O)c(O)c4)c3)CC2)c1] |
| 30 | 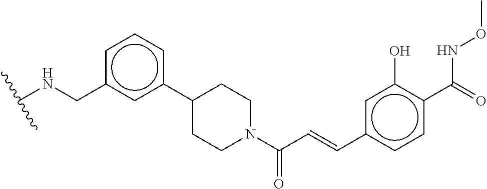 |
| 31 | 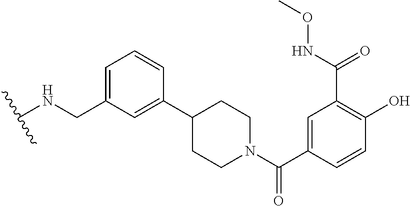 [SMILES: *NCc1cccc(C2CCN(C(=O)c3ccc(O)c(C(=O)NOC)c3)CC2)c1] |
| 32 | 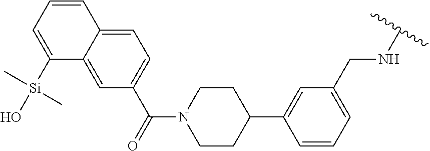 [SMILES: *NCc1cccc(C2CCN(C(=O)c3ccc4cccc([Si](C)(C)O)c4c3)CC2)c1] |
| 33 | 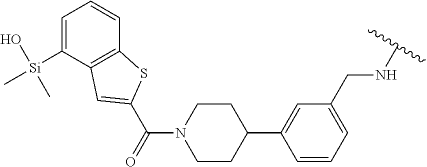 [SMILES: *NCc1cccc(C2CCN(C(=O)c3cc4c([Si](C)(C)O)cccc4s3)CC2)c1] |
| 34 | 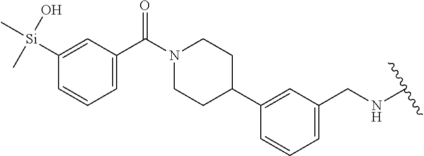 [SMILES: *NCc1cccc(C2CCN(C(=O)c3cccc([Si](C)(C)O)c3)CC2)c1] |
| 35 | 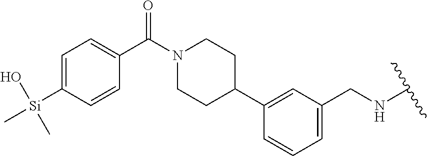 [SMILES: *NCc1cccc(C2CCN(C(=O)c3ccc([Si](C)(C)O)cc3)CC2)c1] |
| 36 | 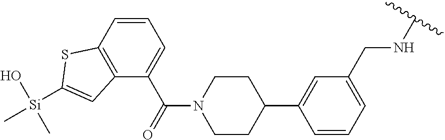 [SMILES: *NCc1cccc(C2CCN(C(=O)c3cccc4sc([Si](C)(C)O)cc34)CC2)c1] |
| 37 | 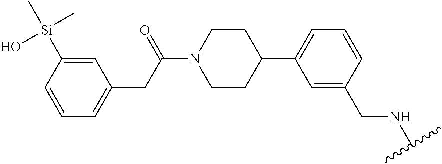 [SMILES: *NCc1cccc(C2CCN(C(=O)Cc3cccc([Si](C)(C)O)c3)CC2)c1] |
| 38 | 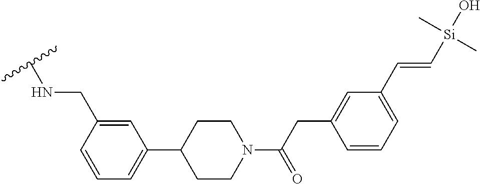 [SMILES: *NCc1cccc(C2CCN(C(=O)Cc3cccc(/C=C/[Si](C)(C)O)c3)CC2)c1] |
| 39 | 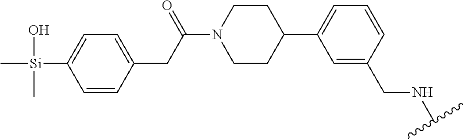 [SMILES: *NCc1cccc(C2CCN(C(=O)Cc3ccc([Si](C)(C)O)cc3)CC2)c1] |

Similar to Scheme Xd, Scheme Xe provides a synthetic procedure for making B derivatives having various connectors of shorter length attached to both the benzodiazepine compound and to any of the above-identified linkers (Z¹, Z², Z³ and Z⁴). In the scheme below, the linker moiety is designated by Z. Activated ester 6 is reacted with various nucleophiles to provide benzodiazepine-connector 2 derivative 8c. For example, Scheme Xe provides for a connector Y (e.g. Y₁, Y₂, Y₃ or Y₄) attached to a linker moiety (Z), wherein Y is —R— (e.g., —R— of 8c).

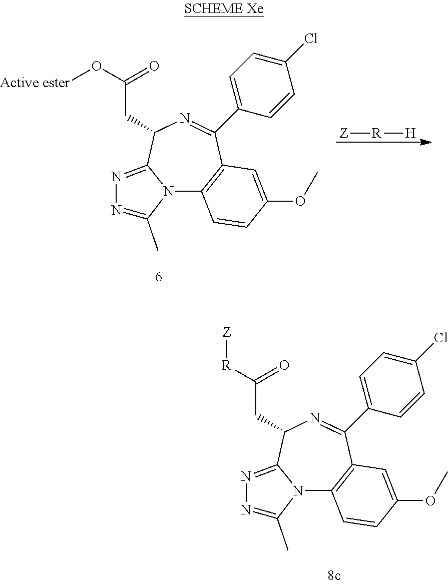

For example, R—Z (i.e., Y—Z) may be represented by the structure:

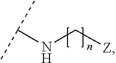

wherein n is 0, 1, 2, 3, 4, or 5, e.g. n is 1 to 5. For example, Scheme Xe provides for a linker Y (e.g. Y₁, Y₂, Y₃ or Y₄).

Scheme Xf provides an additional exemplary synthetic procedure for making B derivatives having various connectors attached to both the benzodiazepine compound and to any of the above-identified linkers (Z¹, Z², Z³ and Z⁴). In the scheme below, the linker moiety is designated by Z. Activated ester 6a is reacted with various nucleophiles to provide benzodiazepine-connector 2 derivative 8d. For example, Scheme Xf provides for a connector Y (e.g. Y₁, Y₂, Y₃ or Y₄) attached to a linker moiety (Z), wherein Y is —NHCH₂—C(O)—R— (e.g., —NHCH₂—C(O)—R— of 8d).

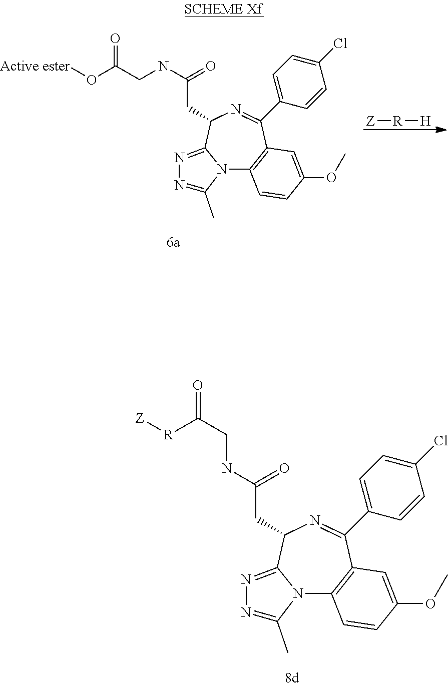

For example, R—Z may be represented by the structure:

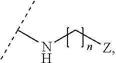

wherein n is 0, 1, 2, 3, 4 or 5, e.g. n is 1 to 5.

Additional examples for Z—R—H and —R—Z that can be utilized in Scheme Xe and Scheme Xf can be found in Table D, seen below:

TABLE D

| No. | Z—R—H | —R—Z |
|---|---|---|
| 1 | 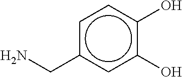 | 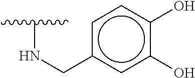 |
| 2 | 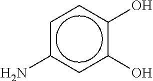 | 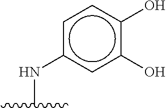 |
| 3 | 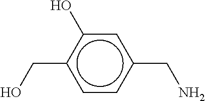 | 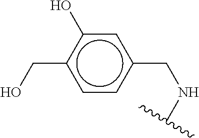 |
| 4 | 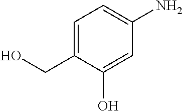 | 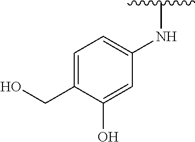 [SMILES: *Nc1ccc(CO)c(O)c1] |
| 5 | 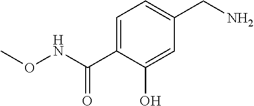 | 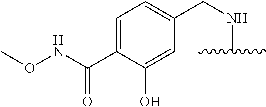 [SMILES: *NCc1ccc(C(=O)NOC)c(O)c1] |
| 6 | 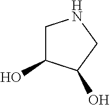 | 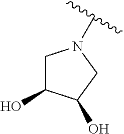 [SMILES: *N1C[C@@H](O)[C@@H](O)C1] |
| 7 | 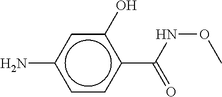 | 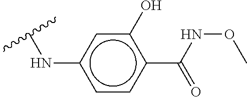 |
| 8 | 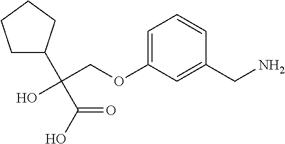 | 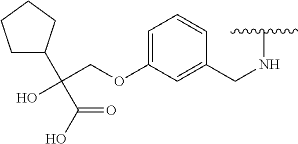 [SMILES: *NCc1cccc(OCC(O)(C(=O)O)C2CCCC2)c1] |
| 9 | 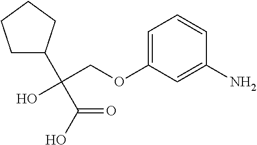 | 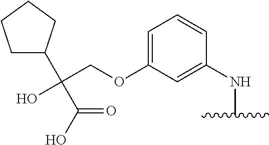 [SMILES: *Nc1cccc(OCC(O)(C(=O)O)C2CCCC2)c1] |
| 10 | 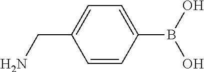 | 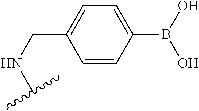 [SMILES: *NCc1ccc(B(O)O)cc1] |
| 11 | 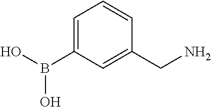 | 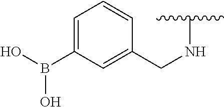 [SMILES: *NCc1cccc(B(O)O)c1] |
| 12 | 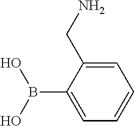 | 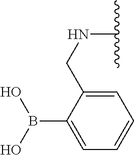 [SMILES: *NCc1ccccc1B(O)O] |
| 13 | 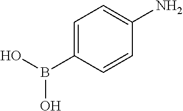 | 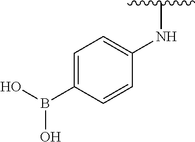 [SMILES: *Nc1ccc(B(O)O)cc1] |
| 14 | 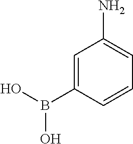 | 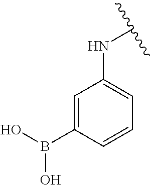 [SMILES: *Nc1cccc(B(O)O)c1] |
| 15 | 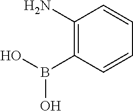 | 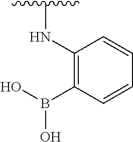 [SMILES: *Nc1ccccc1B(O)O] |
| 16 | 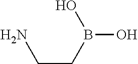 | 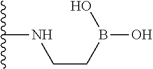 [SMILES: *NCCB(O)O] |
| 17 | 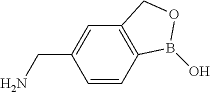 | 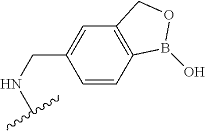 [SMILES: *NCc1ccc2c(c1)COB2O] |
| 18 | 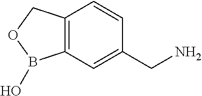 | 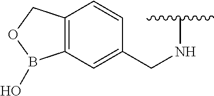 [SMILES: *NCc1ccc2c(c1)B(O)OC2] |
| 19 | 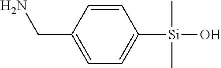 | 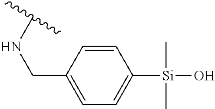 [SMILES: *NCc1ccc([Si](C)(C)O)cc1] |
| 20 | 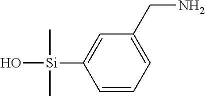 | 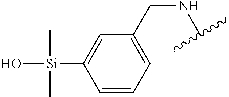 [SMILES: *NCc1cccc([Si](C)(C)O)c1] |
| 21 | 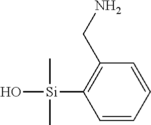 | 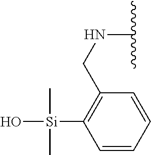 [SMILES: *NCc1ccccc1[Si](C)(C)O] |
| 22 | 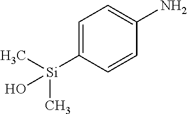 | 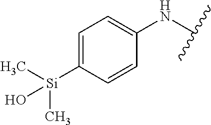 [SMILES: *Nc1ccc([Si](C)(C)O)cc1] |
| 23 | 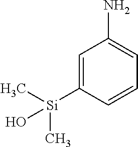 | 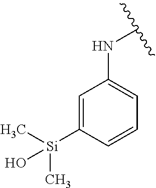 [SMILES: *Nc1cccc([Si](C)(C)O)c1] |
| 24 | 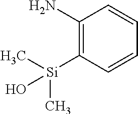 | 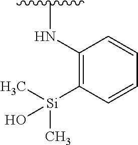 [SMILES: *Nc1ccccc1[Si](C)(C)O] |
| 25 | 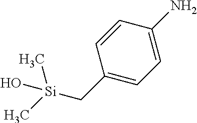 | 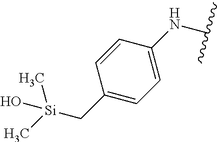 [SMILES: *Nc1ccc(C[Si](C)(C)O)cc1] |
| 26 | 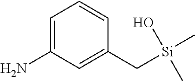 | 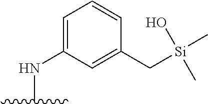 [SMILES: *Nc1cccc(C[Si](C)(C)O)c1] |
| 27 | 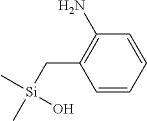 | 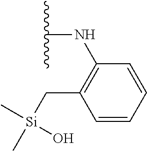 [SMILES: *Nc1ccccc1C[Si](C)(C)O] |
| 28 | 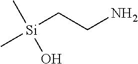 | 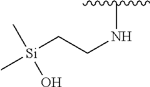 [SMILES: *NCC[Si](C)(C)O] |
| 29 | 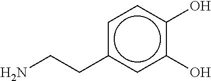 | 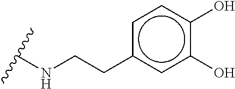 |
| 30 | 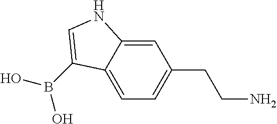 | 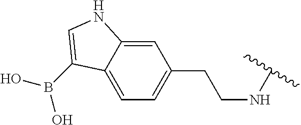 [SMILES: *NCCc1ccc2c(B(O)O)c[nH]c2c1] |
| 31 | 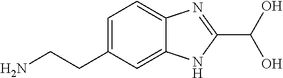 | 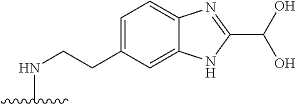 [SMILES: *NCCc1ccc2nc(C(O)O)[nH]c2c1] |
| 32 | 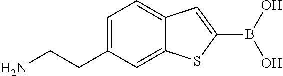 | 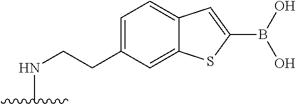 [SMILES: *NCCc1ccc2cc(B(O)O)sc2c1] |
| 33 | 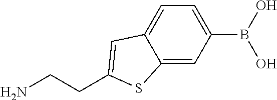 | 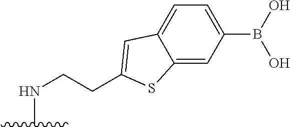 [SMILES: *NCCc1cc2ccc(B(O)O)cc2s1] |
| 34 | 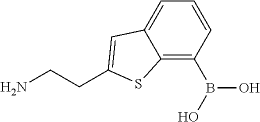 | 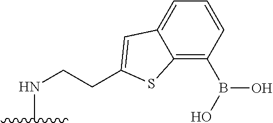 [SMILES: *NCCc1cc2cccc(B(O)O)c2s1] |
| 35 | 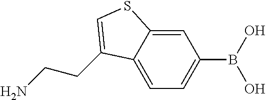 | 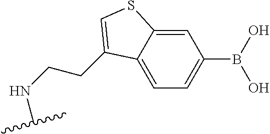 [SMILES: *NCCc1csc2cc(B(O)O)ccc12] |
| 36 | 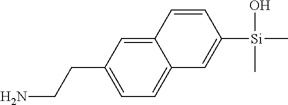 | 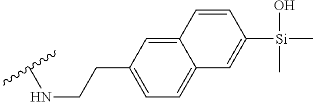 [SMILES: *NCCc1ccc2cc([Si](C)(C)O)ccc2c1] |
| 37 | 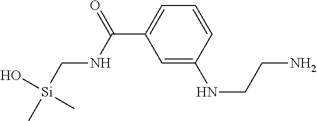 | 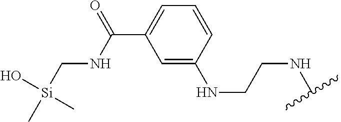 [SMILES: *NCCNc1cccc(C(=O)NC[Si](C)(C)O)c1] |
| 38 | 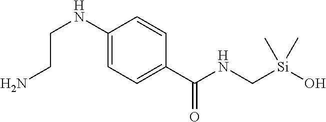 | 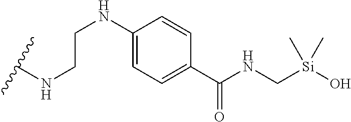 [SMILES: *NCCNc1ccc(C(=O)NC[Si](C)(C)O)cc1] |
| 39 | 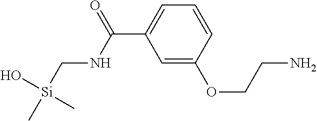 | 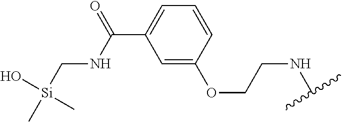 [SMILES: *NCCOc1cccc(C(=O)NC[Si](C)(C)O)c1] |
| 40 | 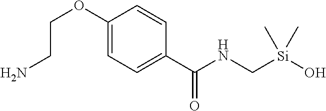 | 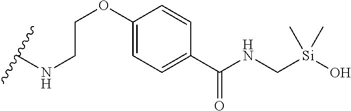 [SMILES: *NCCOc1ccc(C(=O)NC[Si](C)(C)O)cc1] |
| 41 | 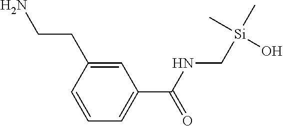 | 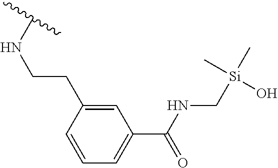 [SMILES: *NCCc1cccc(C(=O)NC[Si](C)(C)O)c1] |
| 42 | 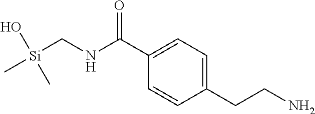 | 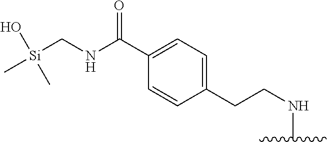 [SMILES: *NCCc1ccc(C(=O)NC[Si](C)(C)O)cc1] |
| 43 | 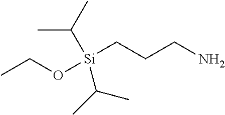 | 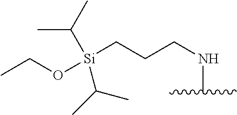 [SMILES: *NCCC[Si](OCC)(C(C)C)C(C)C] |
| 44 | 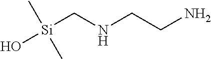 | 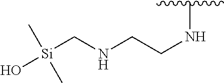 [SMILES: *NCCNC[Si](C)(C)O] |
| 45 | 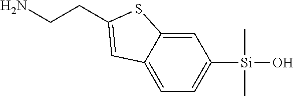 | 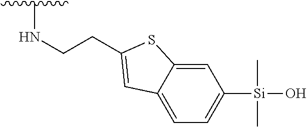 [SMILES: *NCCc1cc2ccc([Si](C)(C)O)cc2s1] |
| 46 | 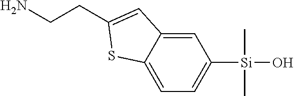 | 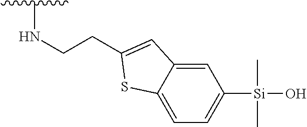 [SMILES: *NCCc1cc2cc([Si](C)(C)O)ccc2s1] |
| 47 | 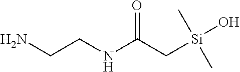 | 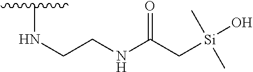 [SMILES: *NCCNC(=O)C[Si](C)(C)O] |
| 48 | 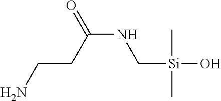 | 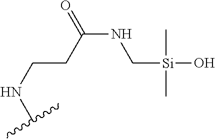 [SMILES: *NCCC(=O)NC[Si](C)(C)O] |
| 49 | 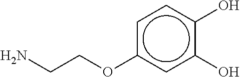 | 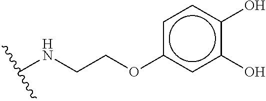 |
| 50 | 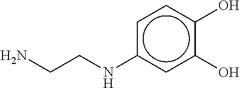 | 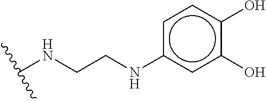 |
| 51 | 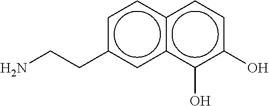 | 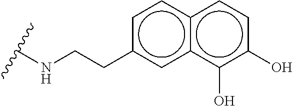 |
| 52 | 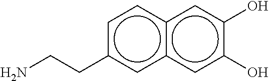 | 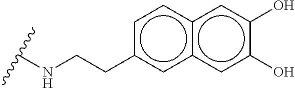 |
| 53 | 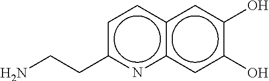 | 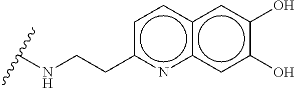 |
| 54 | 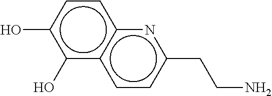 | 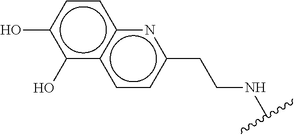 |
| 55 | 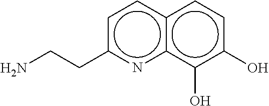 | 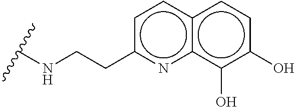 |
| 56 | 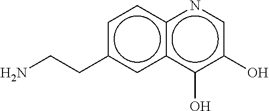 | 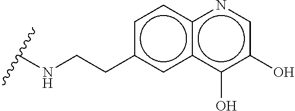 |
| 57 | 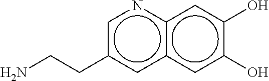 | 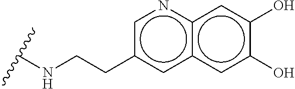 |
| 58 | 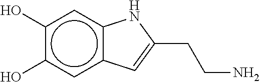 | 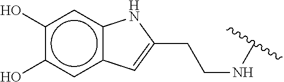 |
| 59 | 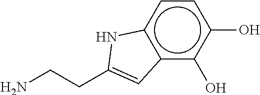 | 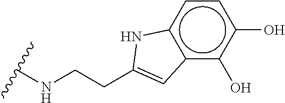 |
| 60 | 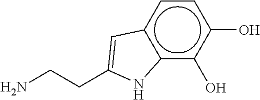 | 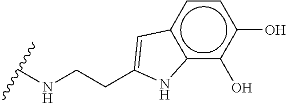 |
| 61 | 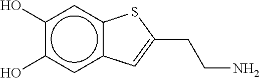 | 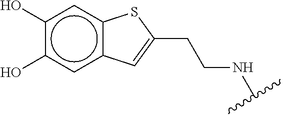 |
| 62 | 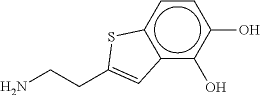 | 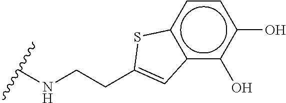 |
| 63 | 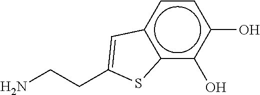 | 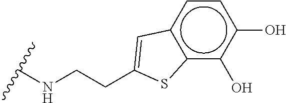 |
| 64 | 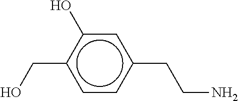 | 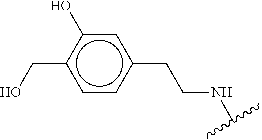 |
| 65 | 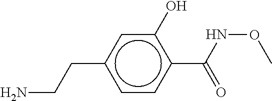 | 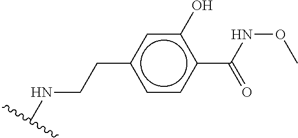 |
| 66 | 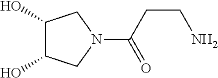 | 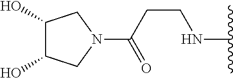 [SMILES: *NCCC(=O)N1C[C@@H](O)[C@@H](O)C1] |
| 67 | 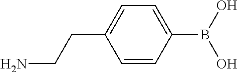 | 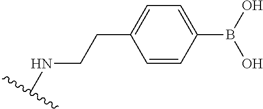 [SMILES: *NCCc1ccc(B(O)O)cc1] |
| 68 | 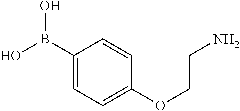 | 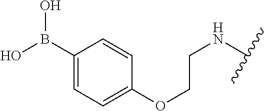 [SMILES: *NCCOc1ccc(B(O)O)cc1] |
| 69 | 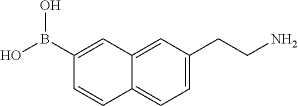 | 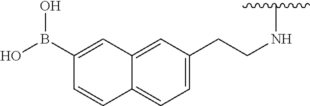 [SMILES: *NCCc1ccc2ccc(B(O)O)cc2c1] |
| 70 | 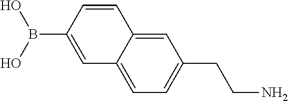 | 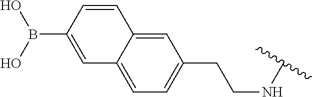 [SMILES: *NCCc1ccc2cc(B(O)O)ccc2c1] |
| 71 | 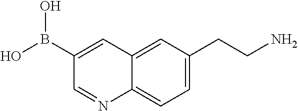 | 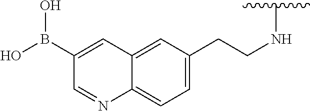 [SMILES: *NCCc1ccc2ncc(B(O)O)cc2c1] |
| 72 | 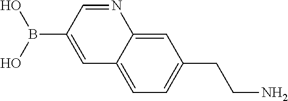 | 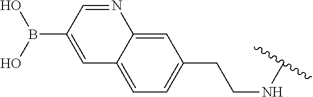 [SMILES: *NCCc1ccc2cc(B(O)O)cnc2c1] |
| 73 | 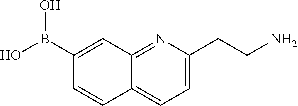 | 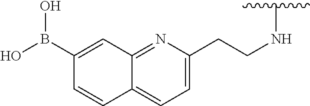 [SMILES: *NCCc1ccc2ccc(B(O)O)cc2n1] |
| 74 | 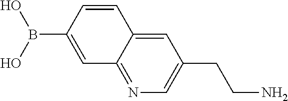 | 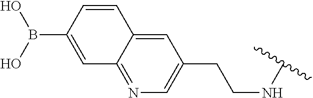 [SMILES: *NCCc1cnc2cc(B(O)O)ccc2c1] |
| 75 | 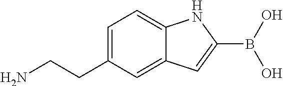 | 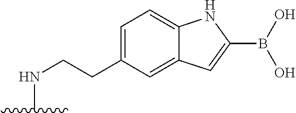 [SMILES: *NCCc1ccc2[nH]c(B(O)O)cc2c1] |
| 76 | 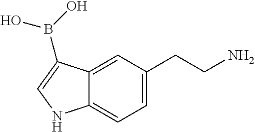 | 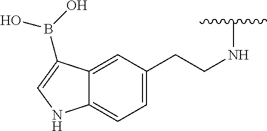 [SMILES: *NCCc1ccc2[nH]cc(B(O)O)c2c1] |
| 77 | 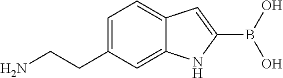 | 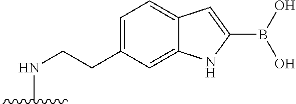 [SMILES: *NCCc1ccc2cc(B(O)O)[nH]c2c1] |
| 78 | 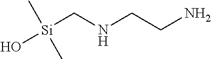 | 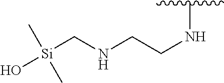 [SMILES: *NCCNC[Si](C)(C)O] |
| 79 | 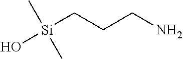 | 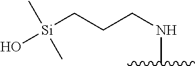 [SMILES: *NCCC[Si](C)(C)O] |
| 80 | 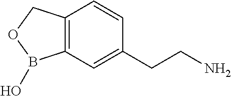 | 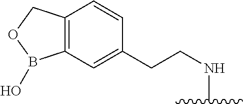 [SMILES: *NCCc1ccc2c(c1)B(O)OC2] |
| 81 | 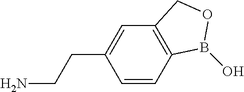 | 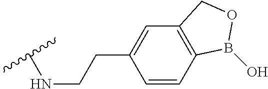 [SMILES: *NCCc1ccc2c(c1)COB2O] |
| 82 | 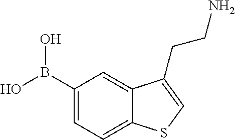 | 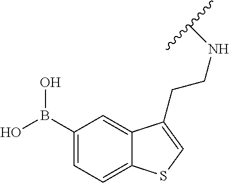 [SMILES: *NCCc1csc2ccc(B(O)O)cc12] |
| 83 | 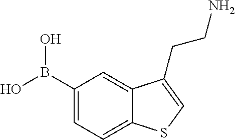 | 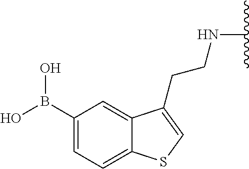 [SMILES: *NCCc1csc2ccc(B(O)O)cc12] |
| 84 | 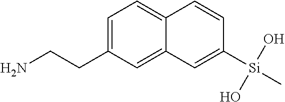 | 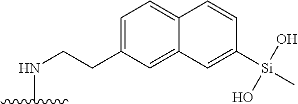 [SMILES: *NCCc1ccc2ccc([Si](C)(O)O)cc2c1] |
| 85 | 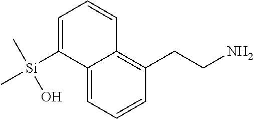 | 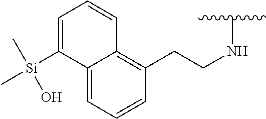 [SMILES: *NCCc1cccc2c([Si](C)(C)O)cccc12] |
| 86 | 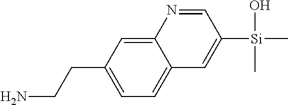 | 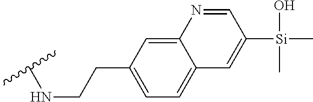 [SMILES: *NCCc1ccc2cc([Si](C)(C)O)cnc2c1] |
| 87 | 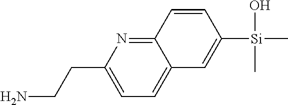 | 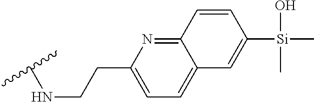 [SMILES: *NCCc1ccc2cc([Si](C)(C)O)ccc2n1] |
| 88 | 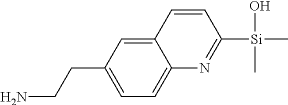 | 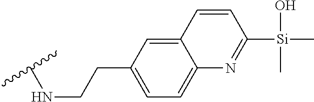 [SMILES: *NCCc1ccc2nc([Si](C)(C)O)ccc2c1] |
| 89 | 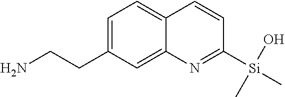 | 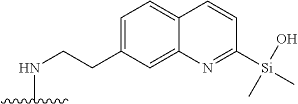 [SMILES: *NCCc1ccc2ccc([Si](C)(C)O)nc2c1] |
| 90 | 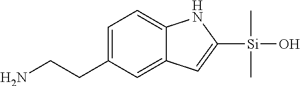 | 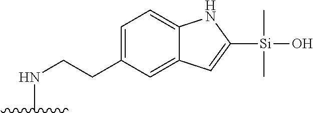 [SMILES: *NCCc1ccc2[nH]c([Si](C)(C)O)cc2c1] |
| 91 | 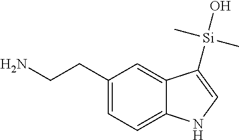 | 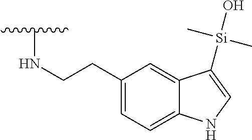 [SMILES: *NCCc1ccc2[nH]cc([Si](C)(C)O)c2c1] |
| 92 | 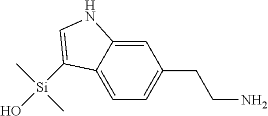 | 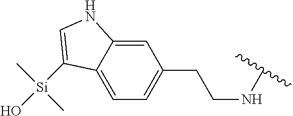 [SMILES: *NCCc1ccc2c([Si](C)(C)O)c[nH]c2c1] |
| 93 | 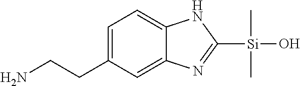 | 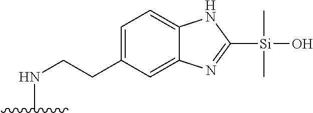 [SMILES: *NCCc1ccc2[nH]c([Si](C)(C)O)nc2c1] |
| 94 | 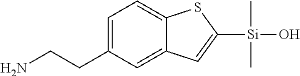 | 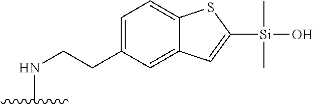 [SMILES: *NCCc1ccc2sc([Si](C)(C)O)cc2c1] |
| 95 | 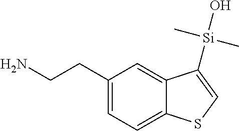 | 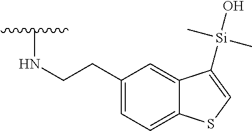 [SMILES: *NCCc1ccc2scc([Si](C)(C)O)c2c1] |
| 96 | 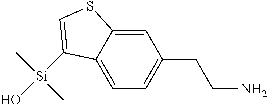 | 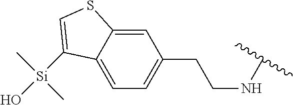 [SMILES: *NCCc1ccc2c([Si](C)(C)O)csc2c1] |

Further to Scheme Xf, Scheme Xg provides an alternative synthetic procedure for making B derivatives having various connectors attached to both the benzodiazepine compound and to any of the above-identified linkers (Z¹, Z², Z³ and Z⁴). In the scheme below, the linker moiety is designated by Z. Activated ester 6a is reacted with Boc-protected ethylenediamine and followed by Boc-removal with TFA to afford diamine 20. The terminal amino group of 20 may be reacted with a variety of electrophiles to afford benzodiazepine-connector 2 derivative 21. For example, Scheme Xg provides for a connector Y (e.g. Y₁, Y₂, Y₃ or Y₄) attached to a linker moiety (Z), wherein Y is —NHCH₂CH₂NH—R— (e.g., —NHCH₂CH₂NH—R— of 21).

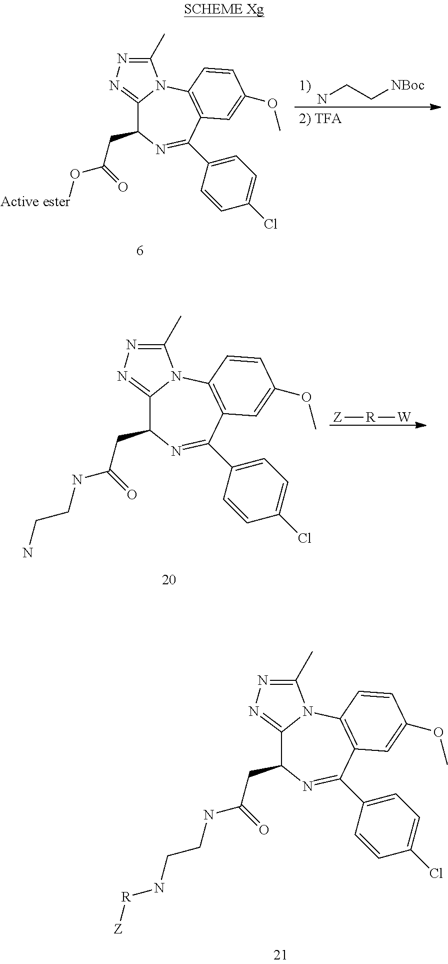

For example, R—Z may be represented by the structure:

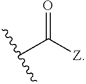

Additional examples for Z—R—W and R—Z that can be utilized in Scheme Xg can be found in Table E, seen below:

TABLE E

| Example No. | Z—R—W | —R—Z |
|---|---|---|
| 1 | 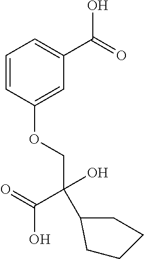 | 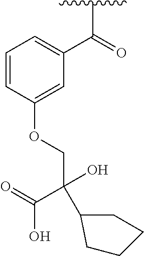 [SMILES: *C(=O)c1cccc(OCC(O)(C(=O)O)C2CCCC2)c1] |
| 2 | 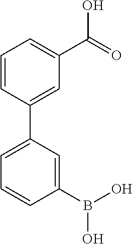 | 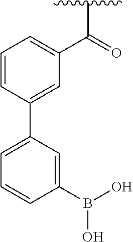 [SMILES: *C(=O)c1cccc(-c2cccc(B(O)O)c2)c1] |
| 3 | 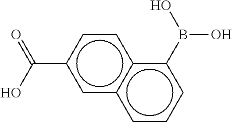 | 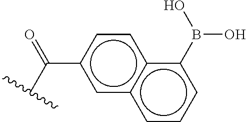 |
| 4 | 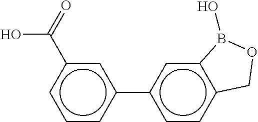 | 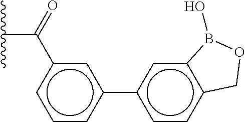 |
| 5 | 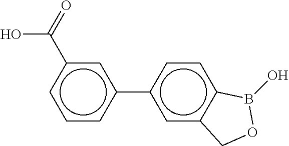 | 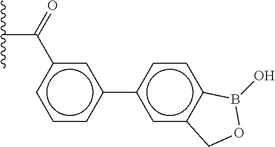 |
| 6 | 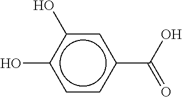 | 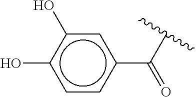 |
| 7 | 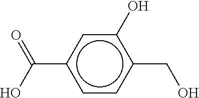 | 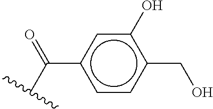 |
| 8 | 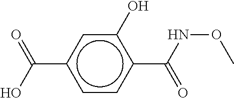 | 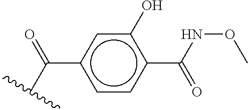 |
| 9 | 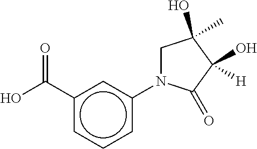 | 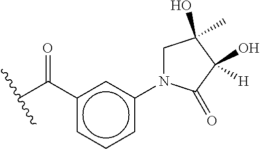 |
| 10 | 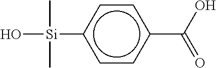 | 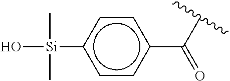 |
| 11 | 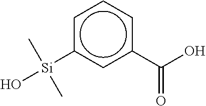 | 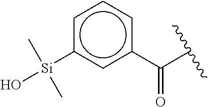 |
| 12 | 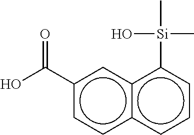 | 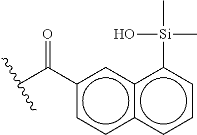 |
| 13 | 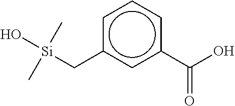 | 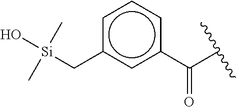 |
| 14 | 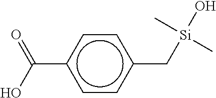 | 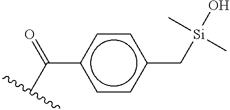 |
| 15 | 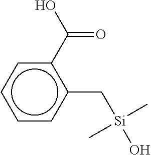 | 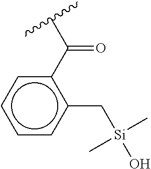 |
| 16 | 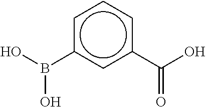 | 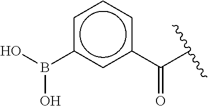 |
| 17 | 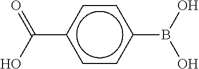 | 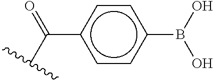 |
| 18 | 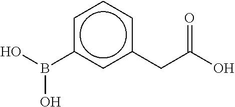 | 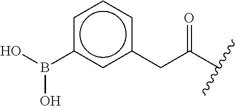 |
| 19 | 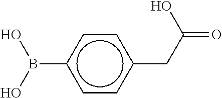 | 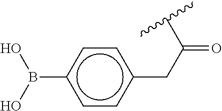 |
| 20 | 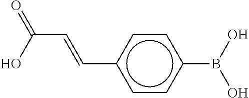 | 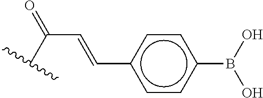 |
| 21 | 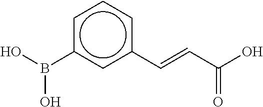 | 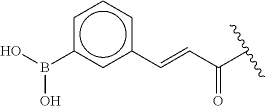 |
| 22 | 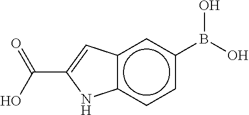 | 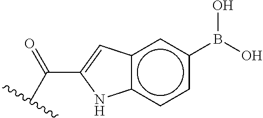 |
| 23 | 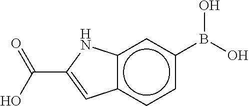 | 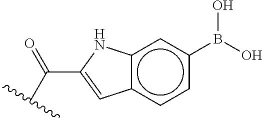 |
| 24 | 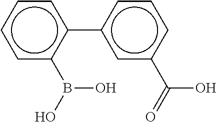 | 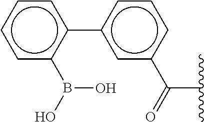 |
| 25 | 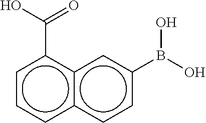 | 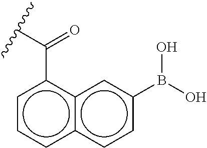 |
| 26 | 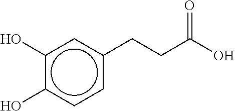 | 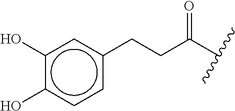 |
| 27 | 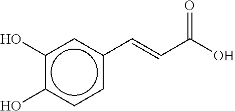 | 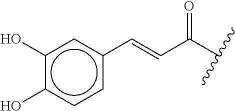 |
| 28 | 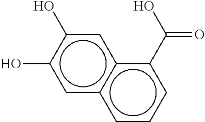 | 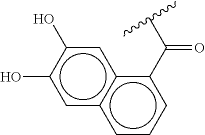 |
| 29 | 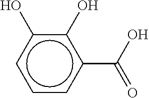 | 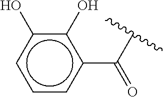 |
| 30 | 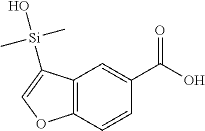 | 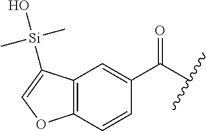 [SMILES: *C(=O)c1ccc2occ([Si](C)(C)O)c2c1] |
| 31 | 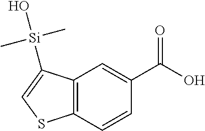 | 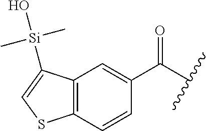 [SMILES: *C(=O)c1ccc2scc([Si](C)(C)O)c2c1] |
| 32 | 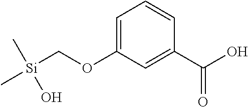 | 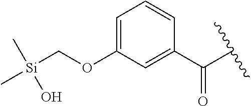 [SMILES: *C(=O)c1cccc(OC[Si](C)(C)O)c1] |
| 33 | 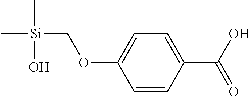 | 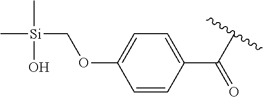 [SMILES: *C(=O)c1ccc(OC[Si](C)(C)O)cc1] |
| 34 | 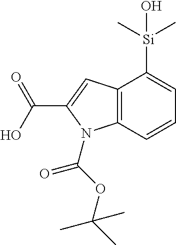 | 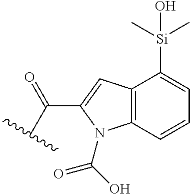 [SMILES: *C(=O)c1cc2c([Si](C)(C)O)cccc2n1C(=O)O] |
| 35 | 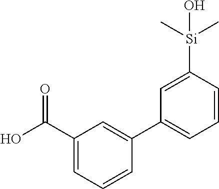 | 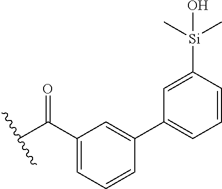 [SMILES: *C(=O)c1cccc(-c2cccc([Si](C)(C)O)c2)c1] |
| 36 | 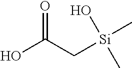 | 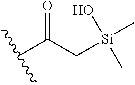 [SMILES: *C(=O)C[Si](C)(C)O] |

In another embodiment, for the above-identified benzodiazepine compounds, there are, e.g., three possible attachment points for the connector element: the phenyl ether, the amino group, or the chloro position of the chlorophenyl ring. As seen below, a connector element may be identified as a Y group in benzodiazepine-connector 1′ A′, benzodiazepine-connector 3 C, and benzodiazepine-connector 4 D:

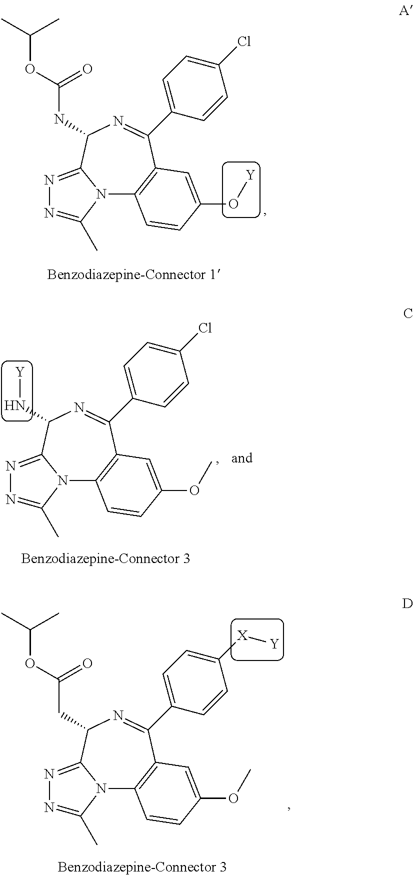

where X=CH₂, S, O, or NH.

For example, Y¹, Y², Y³ and Y⁴ may be Y as described above in connector 1′ A′ or connector 3 C.

In correlation to Scheme Xa, the synthetic route in Scheme Xa′ illustrates a general method for preparing benzodiazepine-connector 1′ derivatives. The method involves attaching the desired substituents to the phenol core. The desired Y group attached at the 4-position of the phenol can be installed by reacting benzodiazepine 3 (see Scheme Xa″) with the appropriate electrophile 5a to provide 4 (benzodiazepine-connector 1′ derivative). For example, Scheme Xa′ provides for a connector Y (e.g. Y₁, Y₂, Y₃ or Y₄).

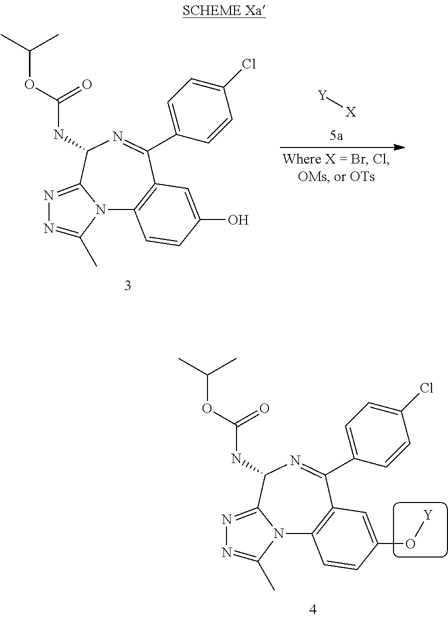

For example, Y may be selected from the group consisting of:

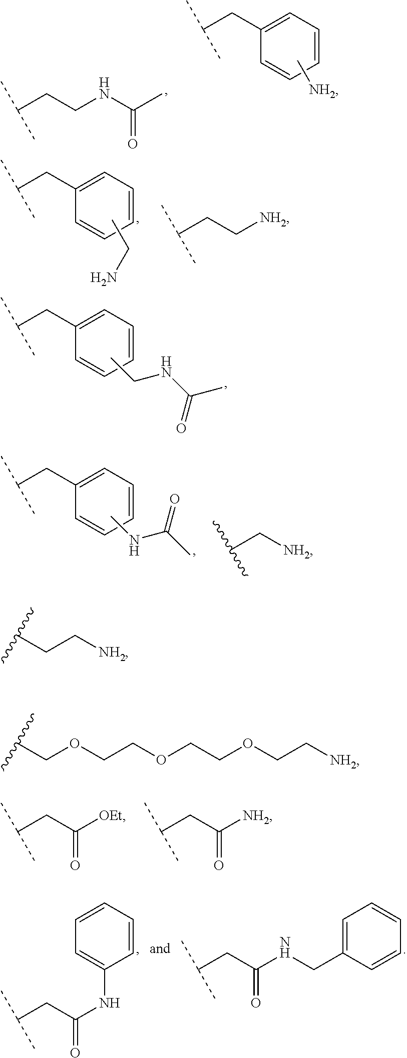

Additional examples for 5a and Y can be found in Table F, seen below:

TABLE F

| No. | 5a | —Y |
|---|---|---|
| 1 | 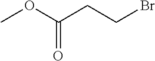 | 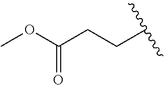 [SMILES: *CCC(=O)OC] |
| 2 | 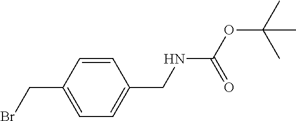 [SMILES: CC(C)(C)OC(=O)NCc1ccc(CBr)cc1] | 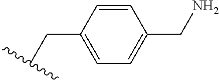 [SMILES: *Cc1ccc(CN)cc1] |
| 3 | 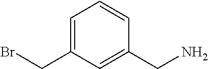 | 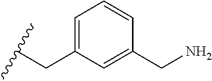 [SMILES: *Cc1cccc(CN)c1] |
| 4 | 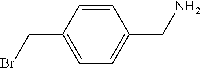 | 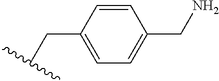 [SMILES: *Cc1ccc(CN)cc1] |
| 5 | 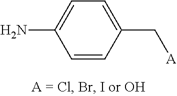 | 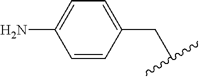 [SMILES: *Cc1ccc(N)cc1] |
| 6 | 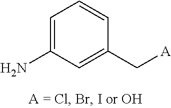 | 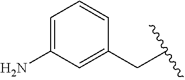 [SMILES: *Cc1cccc(N)c1] |
| 7 | 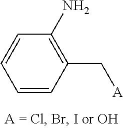 | 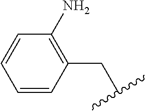 [SMILES: *Cc1ccccc1N] |
| 8 | 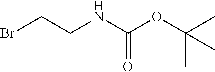 | 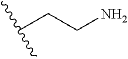 [SMILES: *CCN] |
| 9 | 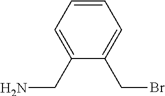 | 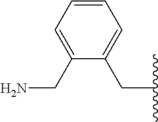 [SMILES: *Cc1ccccc1CN] |
| 10 | 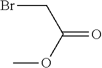 | 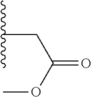 [SMILES: *CC(=O)OC] |
| 11 | 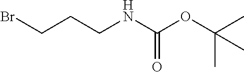 | 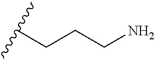 [SMILES: *CCCN] |
| 12 | 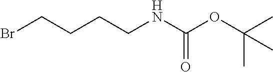 | 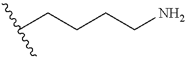 [SMILES: *CCCCN] |
| 13 | 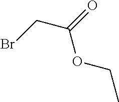 | 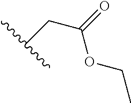 [SMILES: *CC(=O)OCC] |

The synthetic route in Scheme Xb′ illustrates a general method for preparing benzodiazepine-connector 3 derivatives. The method involves attaching the desired carbonyl substituents to the free amine. The carbonyl group can be installed by reacting amine 2 (see Scheme Xa″) with carboxylic acid 7 to provide 6′ (benzodiazepine-connector 3 derivative). For example, Scheme Xb provides for a connector Y (e.g. Y₁, Y₂, Y₃ or Y₄), wherein Y is —C(O)R (e.g., —C(O)R of 6′).

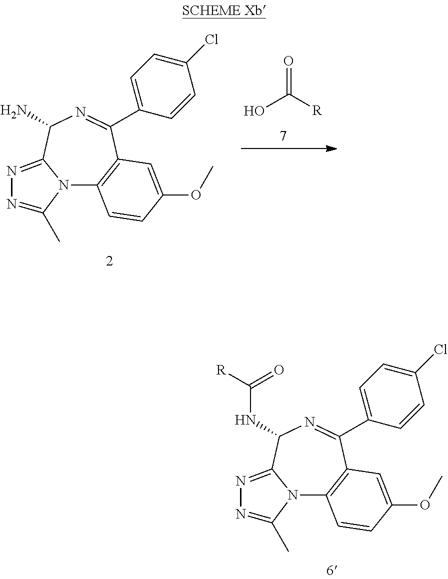

For example, —C(O)R (i.e., Y) may be selected from the group consisting of:

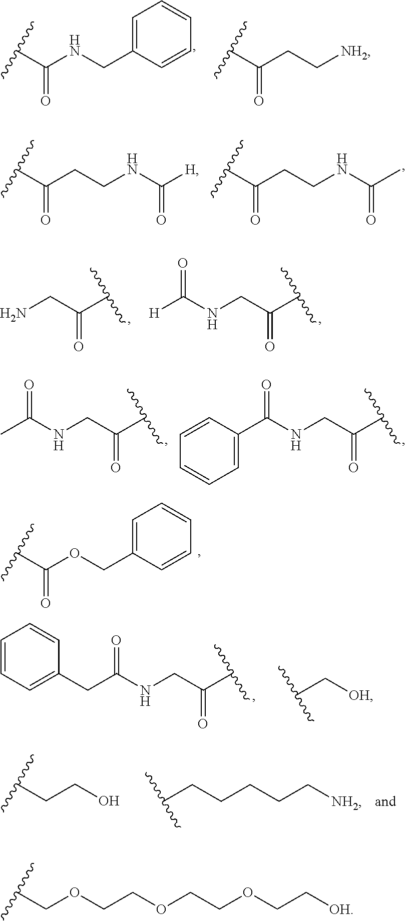

Additional examples for 7 and —C(O)R (i.e., —Y) can be found in Table G, seen below:

TABLE G

| | | —C(O)R |
|---|---|---|
| Example No. | 7 | (i.e., —Y) |
| 1 | 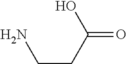 | 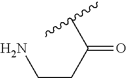 [SMILES: *C(=O)CCN] |
| 2 | 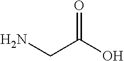 | 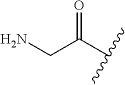 [SMILES: *C(=O)CN] |

The synthetic route in Scheme Xa″ illustrates a general method for preparing benzodiazepine derivatives, for example, benzodiazepine 3, as seen in Scheme Xa′ or, benzodiazepine 2, as seen in Scheme Xb′. The starting material, benzotriazole 1, may be purchased from commercial sources or can be prepared by one of skill in the art, for example, following procedures described in J. Org. Chem. v. 55, p. 2206, 1990. Following the amide coupling of 1 with 1a (to provide 2), ammonia is used to prepare amino-substituted 4. Acid-promoted cyclization (condensation) of 4 affords benzodiazepine carbamate 5. A three step procedure is used to prepare thioamide 8: cleavage of the carbamate 5, Boc-protection of amine 6, and thiolation, utilizing P₄S₁₀ as the sulfur source. The fused triazole 9 is formed from 8 following a three step procedure: hydrazone formation, acylation and cyclization. Boc-group removal from the reaction of 9 with trifluoroacetic acid (TFA) affords the key intermediate 2, which is used to prepare benzodiazepine-connector 3 derivatives. Intermediate 2 is reacted further to prepare phenol 3, which is a key intermediate in the formation of benzodiazepine-connector 1′ derivatives. To this end, cleavage of methyl ether 2 and selective coupling of the free amine affords phenol 3.

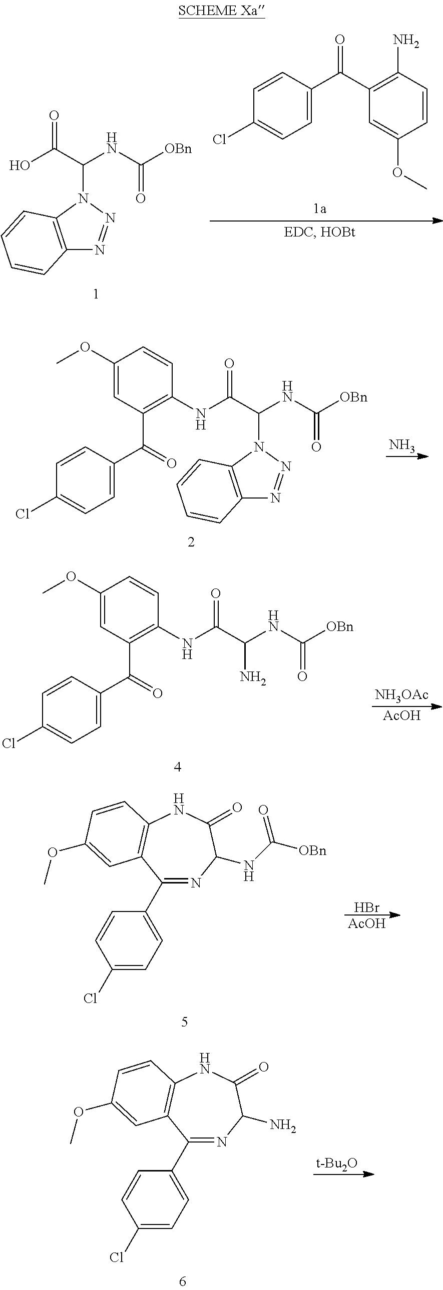

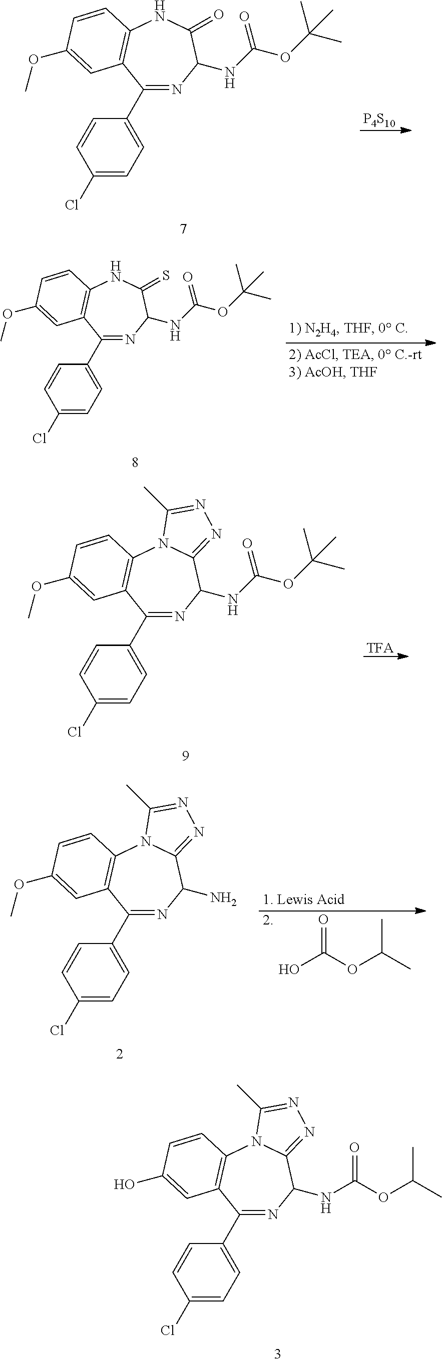

In another embodiment, the two attachment points identified in A′ and C may be further elaborated to incorporate not only a connector moiety (Y), but also a linker (Z).

Scheme Xc′ provides a synthetic procedure for making A′ derivatives having various connectors attached to both the benzodiazepine compound and to any of the above-identified linkers (Z¹, Z², Z³ and Z⁴). In the scheme below, the linker moiety is designated by Z. Phenol 3 is converted to carboxylic acid 9 using ethyl-2-bromoacetate, followed by hydrolysis. Following formation of 9, the general procedure outlined in Scheme Xb can be utilized in the synthesis of the benzodiazepine-connector 1′ derivative 12. For example, Scheme Xc′ provides for a connector Y (e.g. Y₁, Y₂, Y₃ or Y₄) attached to a linker moiety (Z), wherein Y is —CH₂—C(O)—R— (e.g., —CH₂—C(O)—R— of 12).

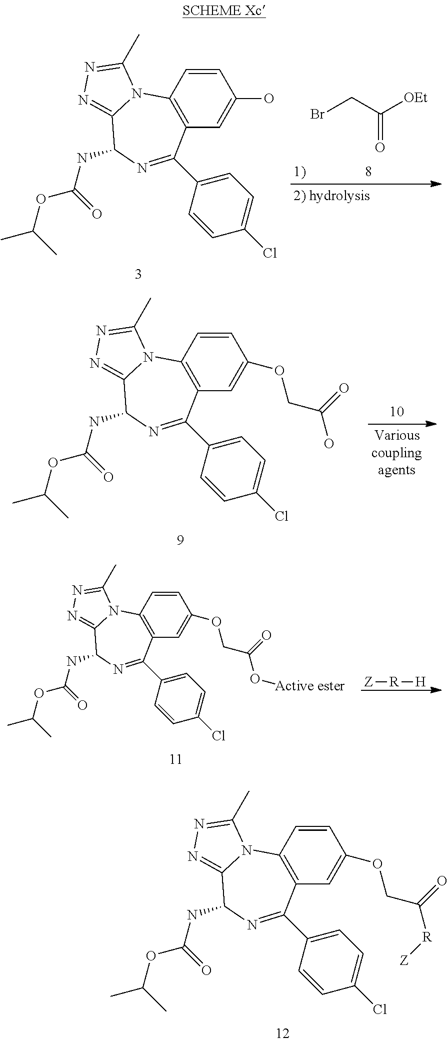

For example, R—Z may be selected from the group consisting of:

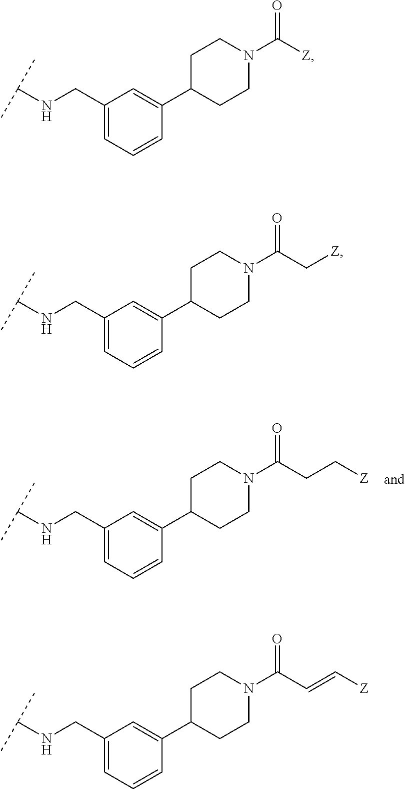

Scheme Xd′ provides an exemplary synthetic procedure for making C derivatives having various connectors attached to both the benzodiazepine compound and to any of the above-identified linkers (Z¹, Z², Z³ and Z⁴). In the scheme below, the linker moiety is designated by Z. Activated ester 14 is prepared following the general procedure seen in Scheme Xc′. Benzodiazepine-connector 3 derivative 15 is afforded by reacting 14 with various nucleophiles. For example, Scheme Xd′ provides for a connector Y (e.g. Y₁, Y₂, Y₃ or Y₄) attached to a linker moiety (Z), wherein Y is —CH₂—C(O)—R— (e.g., —CH₂—C(O)—R— of 15).

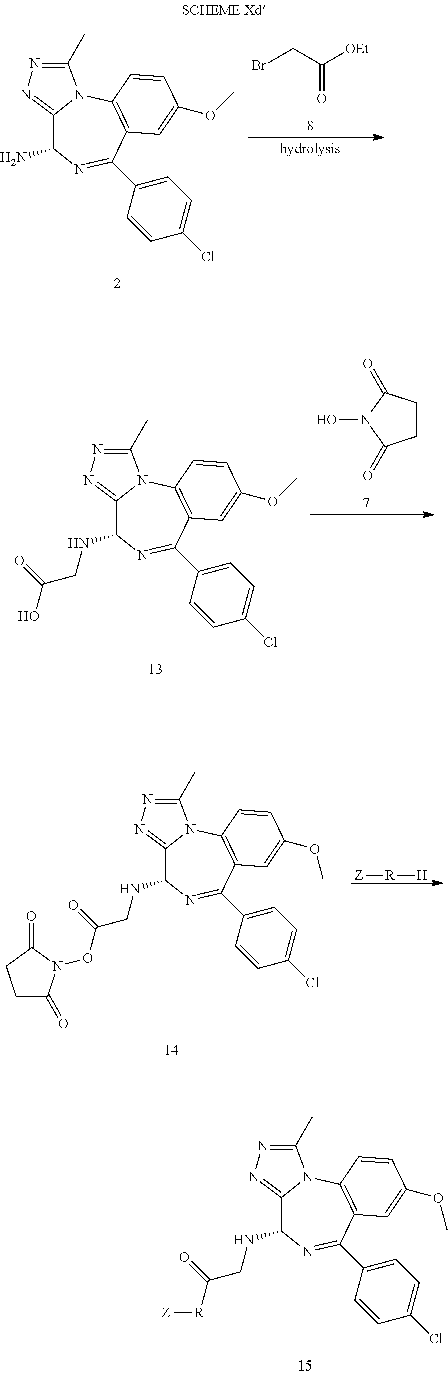

For example, R—Z may be selected from the group consisting of:

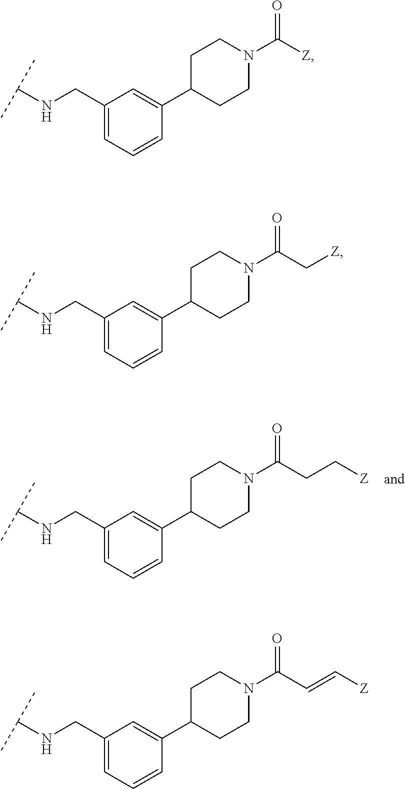

Additional examples for Z—R—H and —R—Z that can be utilized in Scheme Xc′ and Scheme Xd′ can be found in Table H, seen below:

TABLE H

| Example | | |
|---|---|---|
| No. | Z—R—H | —R—Z |
| 1 | 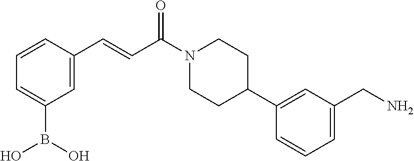 | 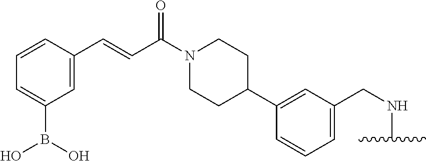 [SMILES: *NCc1cccc(C2CCN(C(=O)/C=C/c3cccc(B(O)O)c3)CC2)c1] |
| 2 | 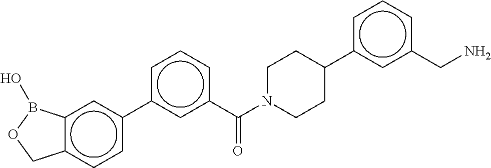 | 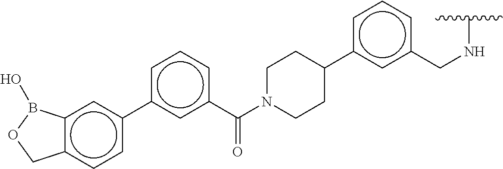 |
| 3 | 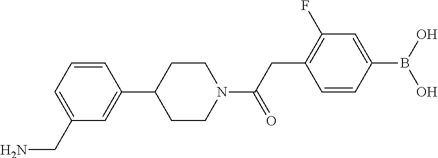 | 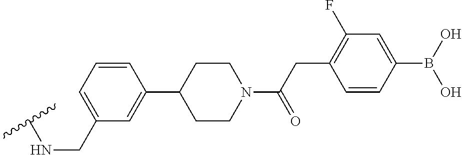 [SMILES: *NCc1cccc(C2CCN(C(=O)Cc3ccc(B(O)O)cc3F)CC2)c1] |
| 4 | 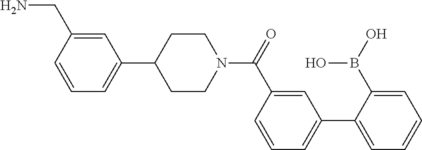 | 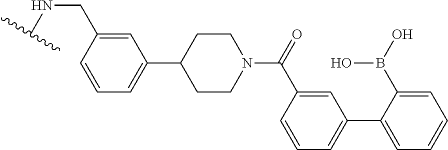 [SMILES: *NCc1cccc(C2CCN(C(=O)c3cccc(-c4ccccc4B(O)O)c3)CC2)c1] |
| 5 | 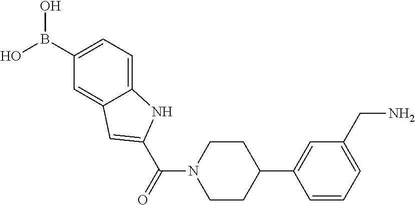 | 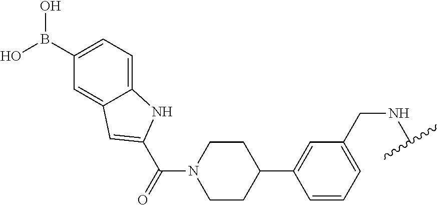 [SMILES: *NCc1cccc(C2CCN(C(=O)c3cc4cc(B(O)O)ccc4[nH]3)CC2)c1] |
| 6 | 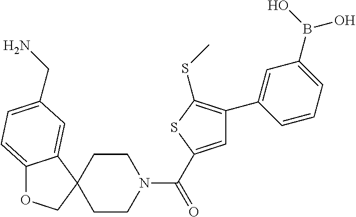 | 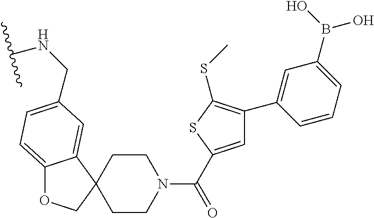 [SMILES: *NCc1ccc2c(c1)C1(CCN(C(=O)c3cc(-c4cccc(B(O)O)c4)c(SC)s3)CC1)CO2] |
| 7 | 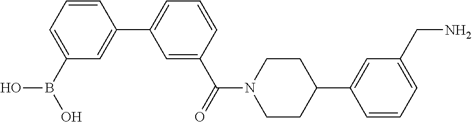 | 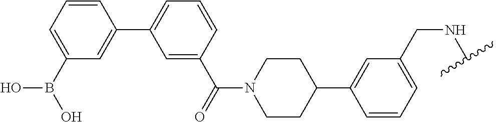 [SMILES: *NCc1cccc(C2CCN(C(=O)c3cccc(-c4cccc(B(O)O)c4)c3)CC2)c1] |
| 8 | 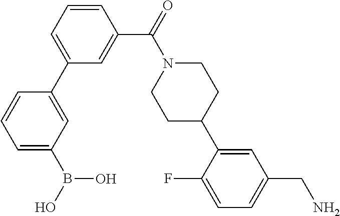 | 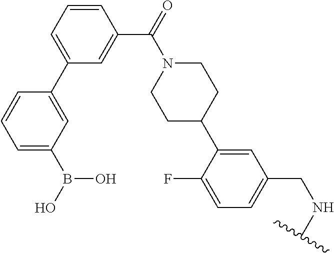 [SMILES: *NCc1ccc(F)c(C2CCN(C(=O)c3cccc(-c4cccc(B(O)O)c4)c3)CC2)c1] |
| 9 | 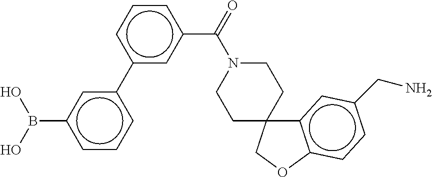 | 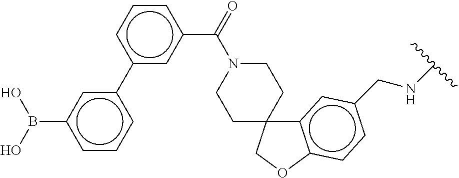 |
| 10 | 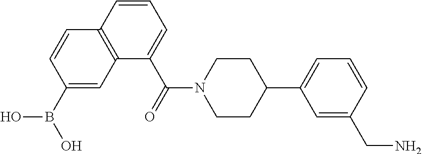 | 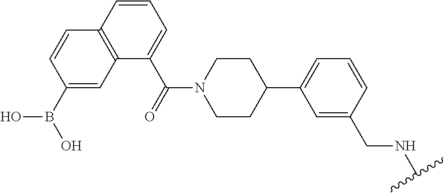 [SMILES: *NCc1cccc(C2CCN(C(=O)c3cccc4ccc(B(O)O)cc34)CC2)c1] |
| 11 | 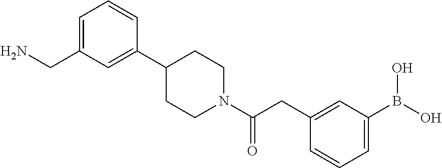 | 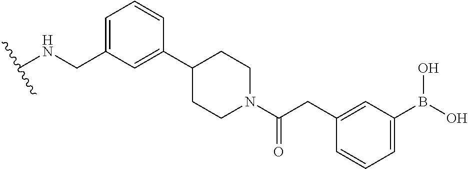 [SMILES: *NCc1cccc(C2CCN(C(=O)Cc3cccc(B(O)O)c3)CC2)c1] |
| 12 | 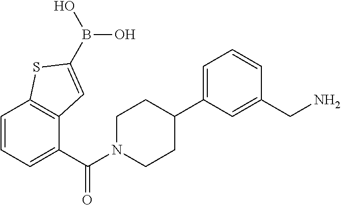 | 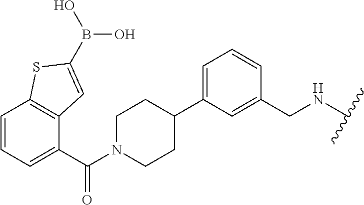 [SMILES: *NCc1cccc(C2CCN(C(=O)c3cccc4sc(B(O)O)cc34)CC2)c1] |
| 13 | 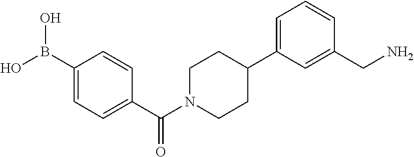 | 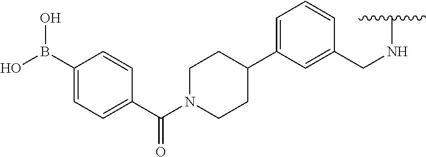 [SMILES: *NCc1cccc(C2CCN(C(=O)c3ccc(B(O)O)cc3)CC2)c1] |
| 14 | 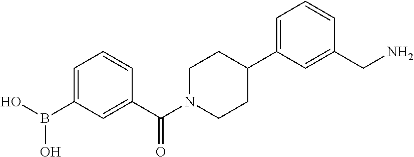 | 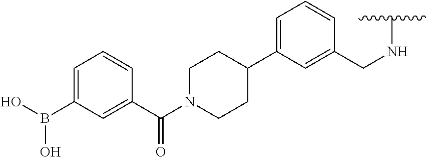 [SMILES: *NCc1cccc(C2CCN(C(=O)c3cccc(B(O)O)c3)CC2)c1] |
| 15 | 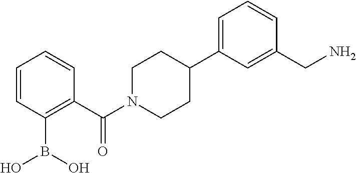 | 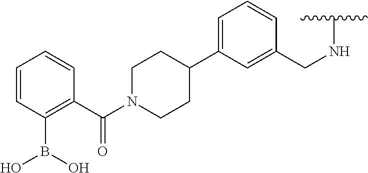 [SMILES: *NCc1cccc(C2CCN(C(=O)c3ccccc3B(O)O)CC2)c1] |
| 16 | 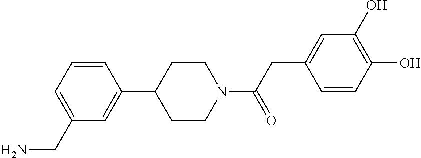 | 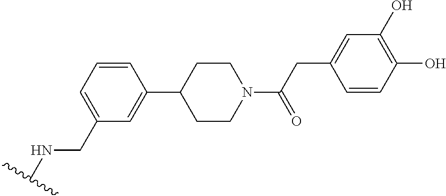 [SMILES: *NCc1cccc(C2CCN(C(=O)Cc3ccc(O)c(O)c3)CC2)c1] |
| 17 | 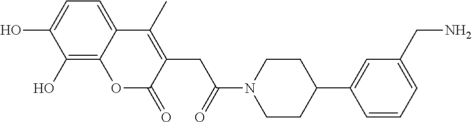 | 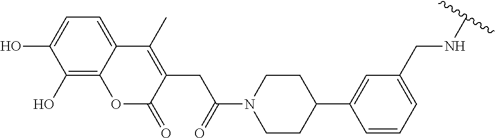 [SMILES: *NCc1cccc(C2CCN(C(=O)Cc3c(C)c4ccc(O)c(O)c4oc3=O)CC2)c1] |
| 18 | 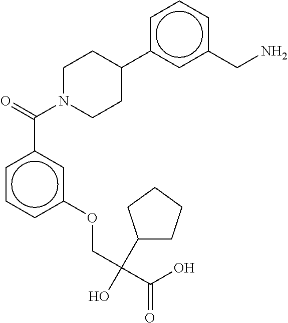 | 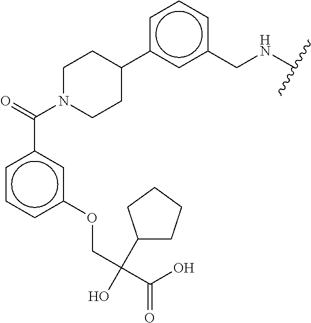 |
| 19 | 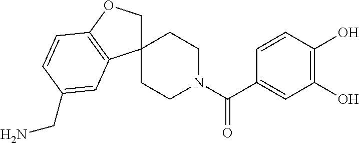 | 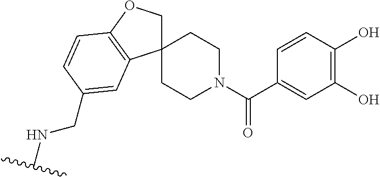 [SMILES: *NCc1ccc2c(c1)C1(CCN(C(=O)c3ccc(O)c(O)c3)CC1)CO2] |
| 20 | 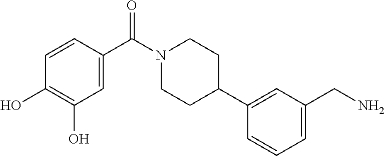 | 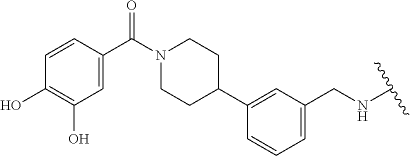 [SMILES: *NCc1cccc(C2CCN(C(=O)c3ccc(O)c(O)c3)CC2)c1] |
| 21 | 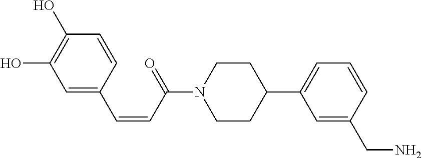 | 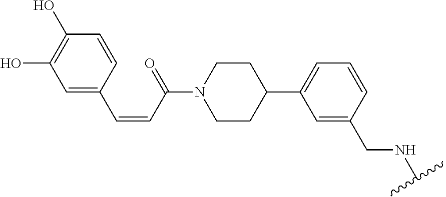 [SMILES: *NCc1cccc(C2CCN(C(=O)/C=C\c3ccc(O)c(O)c3)CC2)c1] |
| 22 | 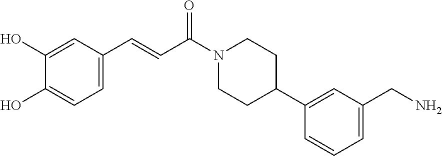 | 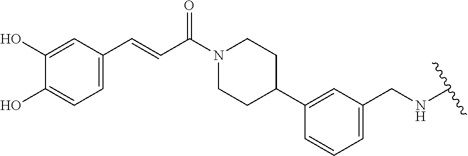 [SMILES: *NCc1cccc(C2CCN(C(=O)/C=C/c3ccc(O)c(O)c3)CC2)c1] |
| 23 | 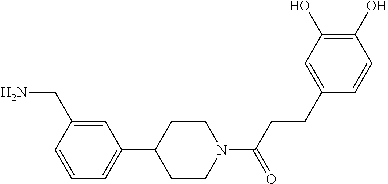 | 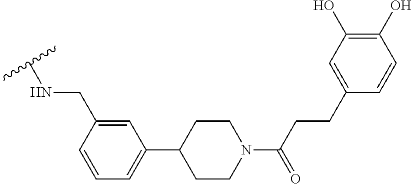 [SMILES: *NCc1cccc(C2CCN(C(=O)CCc3ccc(O)c(O)c3)CC2)c1] |
| 24 | 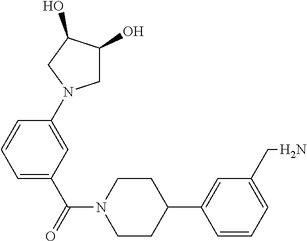 | 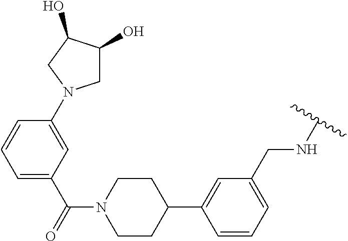 [SMILES: *NCc1cccc(C2CCN(C(=O)c3cccc(N4C[C@@H](O)[C@@H](O)C4)c3)CC2)c1] |
| 25 | 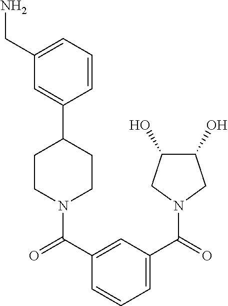 | 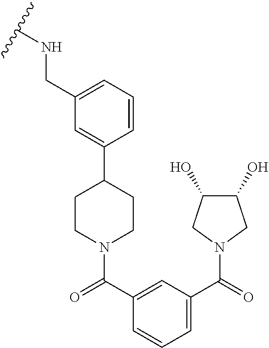 [SMILES: *NCc1cccc(C2CCN(C(=O)c3cccc(C(=O)N4C[C@@H](O)[C@@H](O)C4)c3)CC2)c1] |
| 26 | 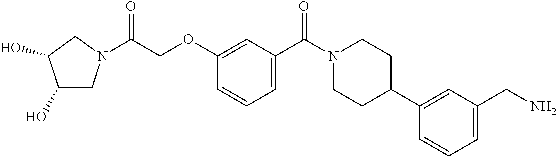 | 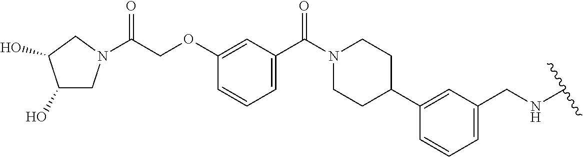 [SMILES: *NCc1cccc(C2CCN(C(=O)c3cccc(OCC(=O)N4C[C@@H](O)[C@@H](O)C4)c3)CC2)c1] |
| 27 | 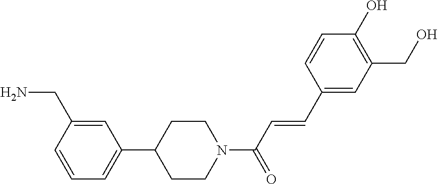 | 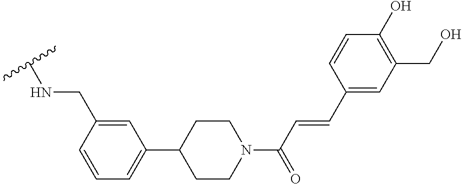 [SMILES: *NCc1cccc(C2CCN(C(=O)/C=C/c3ccc(O)c(CO)c3)CC2)c1] |
| 28 | 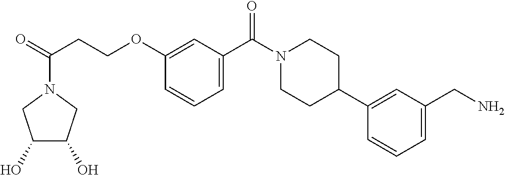 | 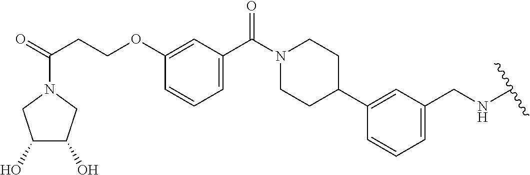 [SMILES: *NCc1cccc(C2CCN(C(=O)c3cccc(OCCC(=O)N4C[C@@H](O)[C@@H](O)C4)c3)CC2)c1] |
| 29 | 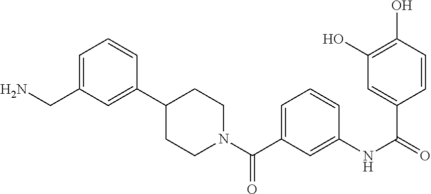 | 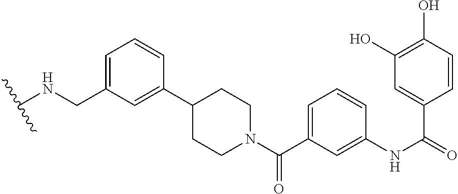 [SMILES: *NCc1cccc(C2CCN(C(=O)c3cccc(NC(=O)c4ccc(O)c(O)c4)c3)CC2)c1] |
| 30 | 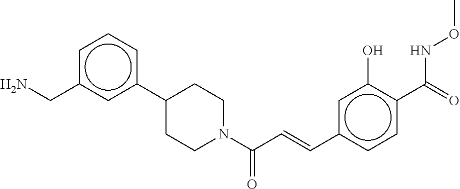 | 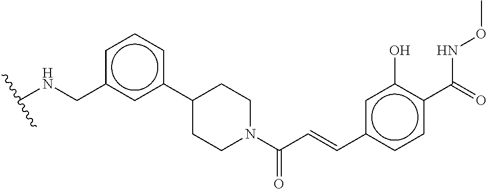 |
| 31 | 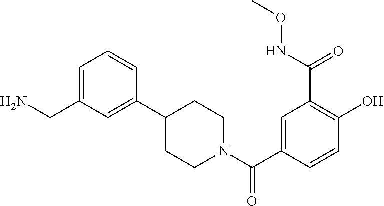 | 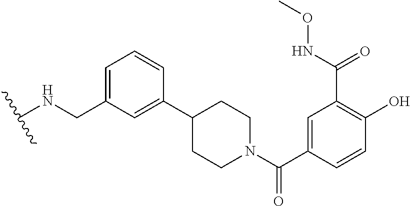 [SMILES: *NCc1cccc(C2CCN(C(=O)c3ccc(O)c(C(=O)NOC)c3)CC2)c1] |

Scheme Xe′ provides a synthetic procedure for making C derivatives having various connectors of shorter length attached to both the benzodiazepine compound and to any of the above-identified linkers (Z¹, Z², Z³ and Z⁴). In the scheme below, the linker moiety is designated by Z. Amine intermediate 2 is reacted with various electrophiles, for example, a carboxylic acid, to provide benzodiazepine-connector 3 derivative 17. For example, Scheme Xe′ provides for a connector Y (e.g. Y₁, Y₂, Y₃ or Y₄) attached to a linker moiety (Z), wherein Y is —R— (e.g., —R— of 17).

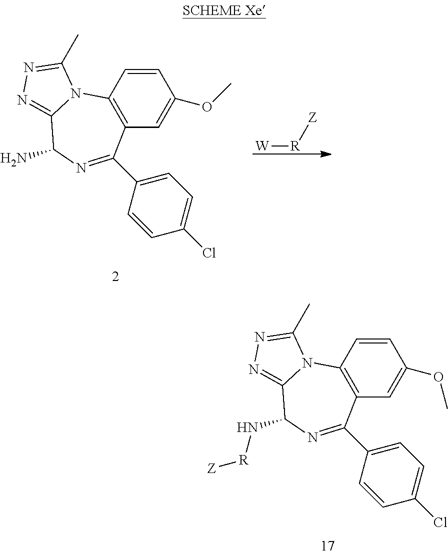

For example, R—Z (e.g., Y—Z) may be represented by the structure:

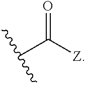

Further to Scheme Xe′, Scheme Xf provides a synthetic procedure for making C derivatives having various connectors of longer length attached to both the benzodiazepine compound and to any of the above-identified linkers (Z¹, Z², Z³ and Z⁴). In the scheme below, the linker moiety is designated by Z. Amine intermediate 2 is reacted with various carboxylic acids to provide benzodiazepine-connector 3 derivative 20. For example, Scheme Xf provides for a connector Y (e.g. Y₁, Y₂, Y₃ or Y₄) attached to a linker moiety (Z), wherein Y is —C(O)CH₂—NHR— (e.g., —C(O)CH₂—NHR— of 20).

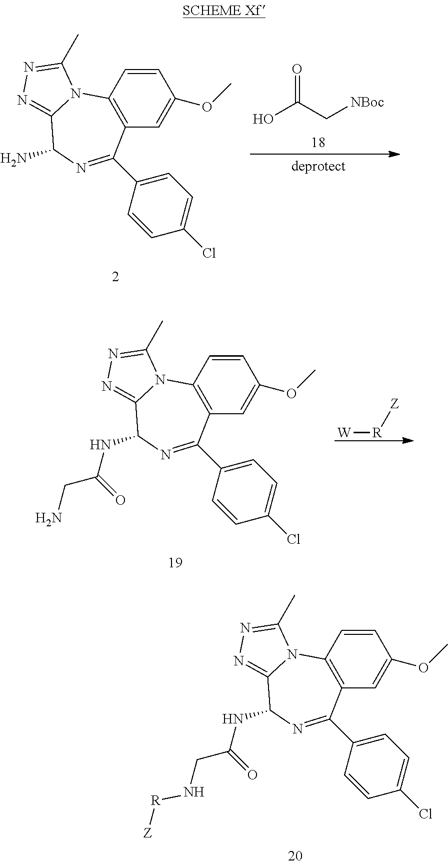

For example, R—Z may be represented by the structure:

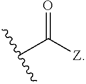

Additional examples for Z—R—W and —R—Z that can be utilized in Scheme Xe′ and Scheme Xf′ can be found in Table I, seen below:

TABLE I

| Example No. | Z—R—W | —R—Z |
|---|---|---|
| 1 | 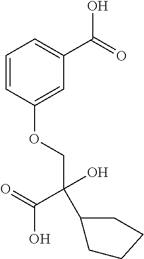 | 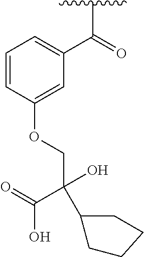 [SMILES: *C(=O)c1cccc(OCC(O)(C(=O)O)C2CCCC2)c1] |
| 2 | 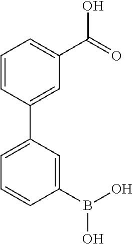 | 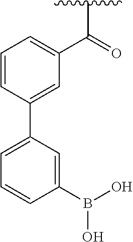 [SMILES: *C(=O)c1cccc(-c2cccc(B(O)O)c2)c1] |
| 3 | 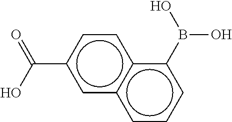 | 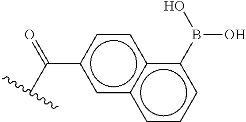 |
| 4 | 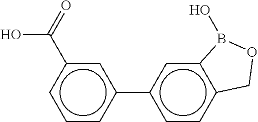 | 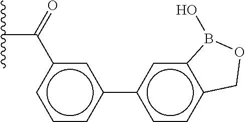 |
| 5 | 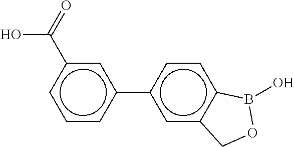 | 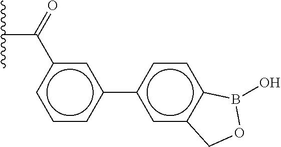 |
| 6 | 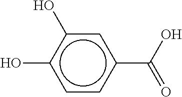 | 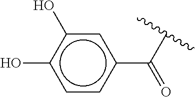 |
| 7 | 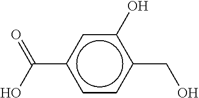 | 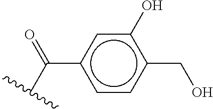 |
| 8 | 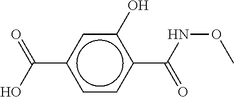 | 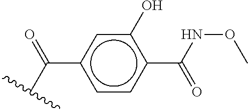 |
| 9 | 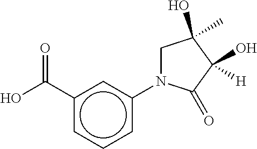 | 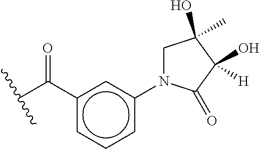 |
| 10 | 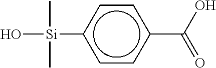 | 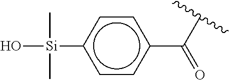 |
| 11 | 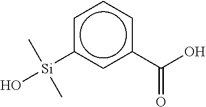 | 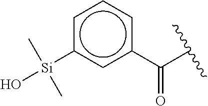 |
| 12 | 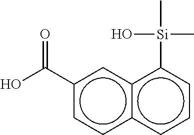 | 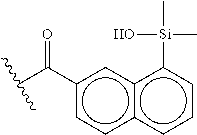 |
| 13 | 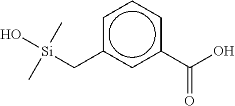 | 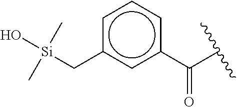 |
| 14 | 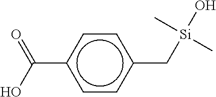 | 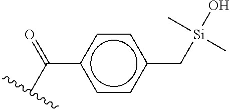 |
| 15 | 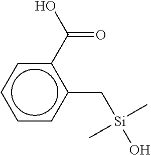 | 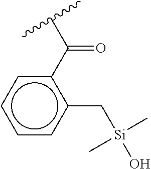 |
| 16 | 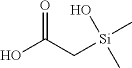 | 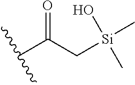 [SMILES: *C(=O)C[Si](C)(C)O] |

In a certain embodiment, for the above-identified benzodiazepine compounds, the attachment point for a connector element of benzodiazepine-connector 2 B is utilized in benzodiazepine-connector 2″ B″:

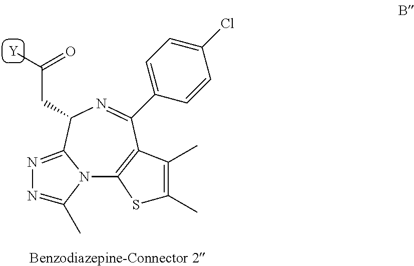

Scheme Xb′ provides a synthetic procedure for making key intermediate 6b. The intermediate (+)-JQ1 may be prepared, for example, by known methods. The activated ester 6b can be prepared by reacting (+)-JQ1 with a coupling reagent, such as EDC or HOBt.

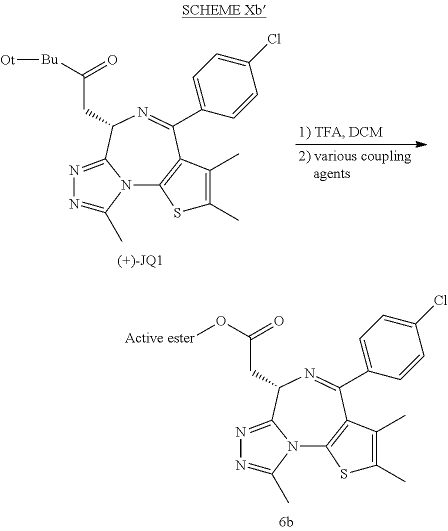

It is contemplated herein that the general methods seen above in Scheme Xb and Schemes Xd-Xg can also utilize intermediate 6b, in place of intermediate 6 or 6a, in the preparation of B′ derivatives.

In one embodiment, an exemplary B′ derivative is represented by the structure:

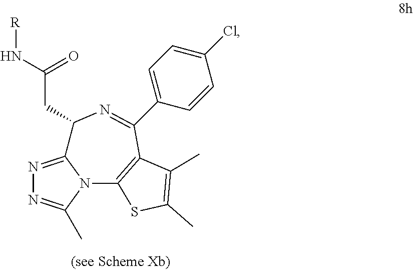

wherein R is, for example, selected from the group consisting of:

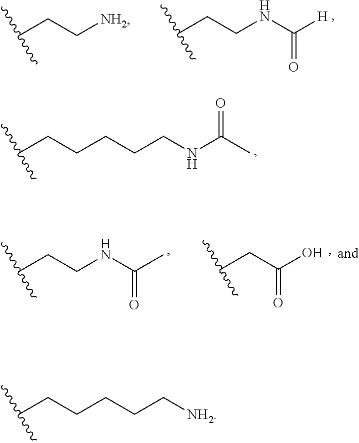

For example, 8h provides for a connector Y (e.g. Y₁, Y₂, Y₃ or Y₄) wherein Y is —NH—R.

In another embodiment, an exemplary B′ derivative is represented by the structure:

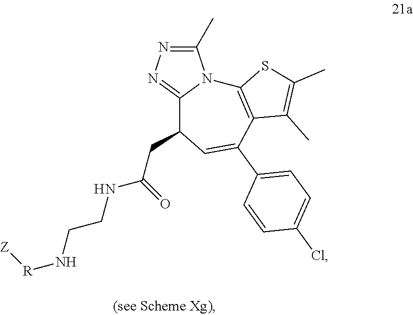

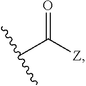

wherein R—Z is, for example, For example, 21a provides for a connector Y (e.g. Y₁, Y₂, Y₃ or Y₄) attached to a linker moiety (Z), wherein Y is —NHCH₂CH₂NH—R—.

For example, an exemplary B′ derivative is represented by the structure:

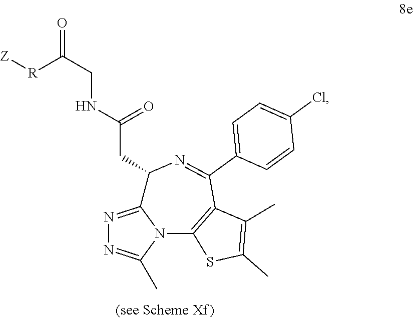

wherein R—Z is, for example,

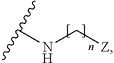

wherein n is 0, 1, 2, 3, 4 or 5, e.g. n is 1 to 5. For example, 8e provides for a connector Y (e.g. Y₁, Y₂, Y₃ or Y₄) attached to a linker moiety (Z), wherein Y is —NHCH₂C(O)R—.

In a certain embodiment, an exemplary B′ derivative is represented by the structure:

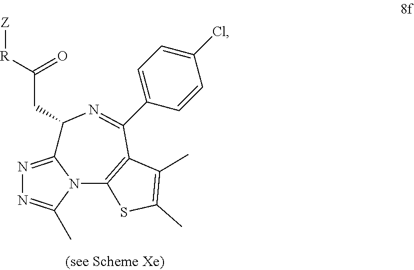

wherein R—Z is, for example,

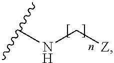

wherein n is 0, 1, 2, 3, 4 or 5, e.g. n is 1 to 5. For example, 8f provides for a connector Y (e.g. Y₁, Y₂, Y₃ or Y₄) attached to a linker moiety (Z), wherein Y is —R—.

In another embodiment, an exemplary B′ derivative is represented by the structure:

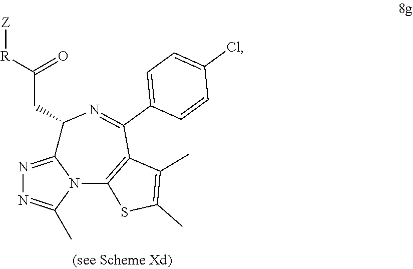

wherein R—Z is, for example, selected from the group consisting of:

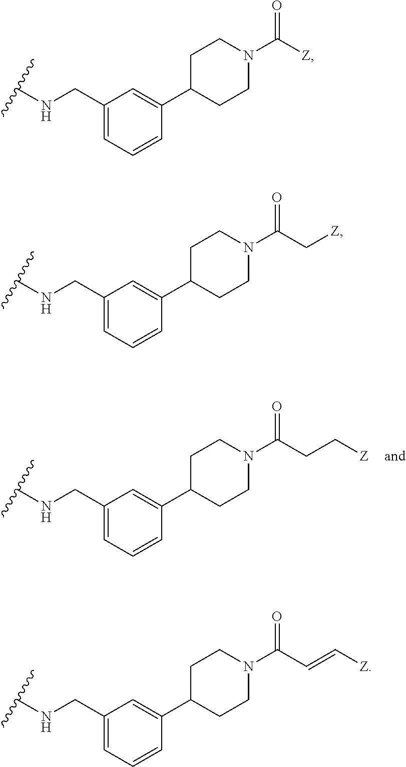

For example, 8g provides for a connector Y (e.g. Y₁, Y₂, Y₃ or Y₄) attached to a linker moiety (Z), wherein Y is —R—.

It will be appreciated that fbr the above-identified tetrahydroquinoline compounds, the connector element may attach at one of at least two possible attachment points for example, via a terminal amino group or via a carbonyl substituent. As seen below, a connector element may be identified as a Y group in tetrahydoquinoline-connector 1 10A′, tetrahydoquinoline-connector 1 10B′ and tetrahydroquinoline-connector 2 10C:

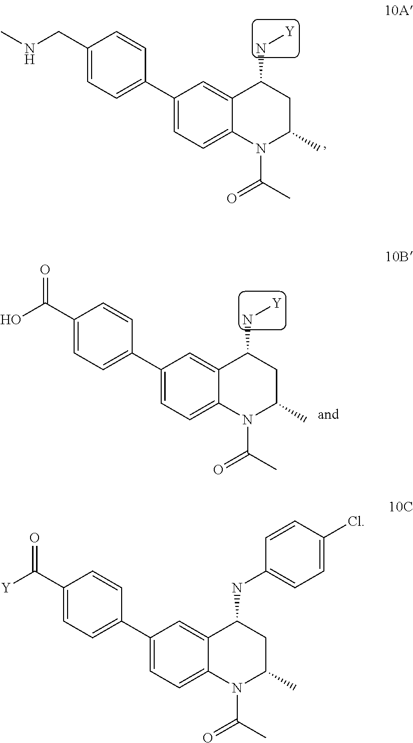

For example, Y¹, Y², Y³ and Y⁴ may be Y as described above in connector 1 10A′ connector 1 10B′ or connector 2 10C.

The synthetic route in Scheme Xh illustrates a divergent procedure for preparing tetrahydroquinoline-connector 1 derivatives. The tetrahydroquinoline core is formed in a two step-process beginning with the condensation of 5, 6 and acetaldehyde to form 7 and followed by conjugate addition to acrylaldehyde to afford 8. Tetrahydroquinoline 8 is utilized in a divergent step to install varying phenyl substituents via reaction with the bromo-group to provide 9A and 9B. Following hydrolysis of the amide group, the desired Y group is attached at the terminal amino group by reacting the unsubstituted amines of 4A or 3 with the appropriate electrophile to provide 10A or 10B (tetrahydroquinoline-connector 1 derivative). For example, Scheme Xh provides for a connector Y (e.g. Y₁, Y₂, Y₃ or Y₄).

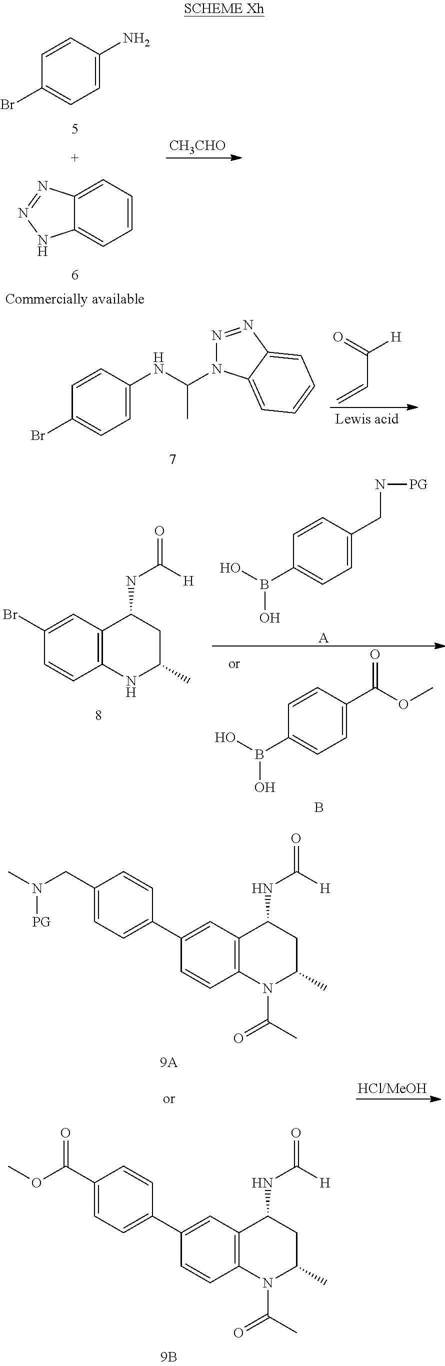

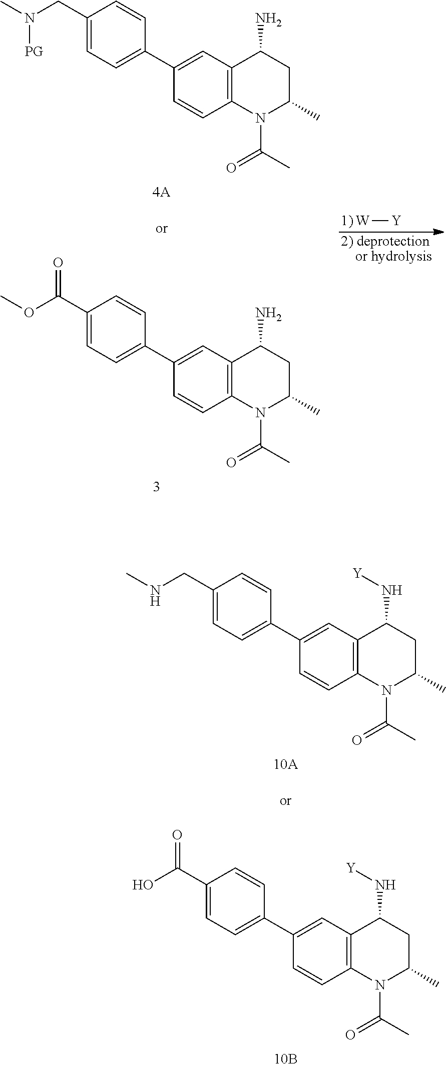

For example, W—Y may be selected from the group consisting of:

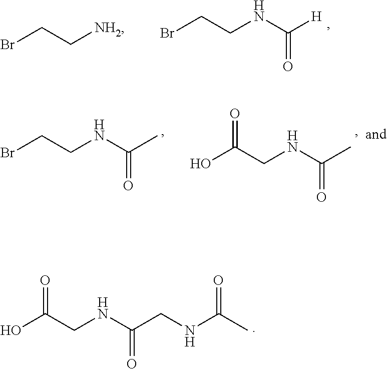

Additional examples for W—Y and —Y can be found in Table J, seen below:

TABLE J

| Example | | |
|---|---|---|
| No. | W—Y | —Y |
| 1 | 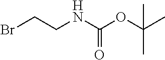 | 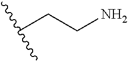 [SMILES: *CCN] |
| 2 | 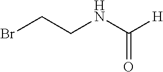 | 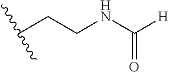 [SMILES: *CCNC=O] |
| 3 | 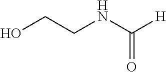 | 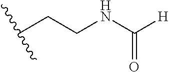 [SMILES: *CCNC=O] |

The synthetic route in Scheme Xi illustrates a general method for preparing tetrahydroquinoline-connector 2 derivatives. Tetrahydroquinoline 3 is converted to phenyl-substituted 11 utilizing a Suzuki coupling, and the ester of 11 is hydrolyzed to afford carboxylic acid 2. The connecter moieties can be installed via a peptide coupling of the carboxylic acid 2 to prepare 12 (tetrahydroquinoline-connector 2 derivatives 10C). For example, Scheme Xi provides for a connector Y (e.g. Y₁, Y₂, Y₃ or Y₄), wherein Y is —W—R (e.g., —W—R of 12).

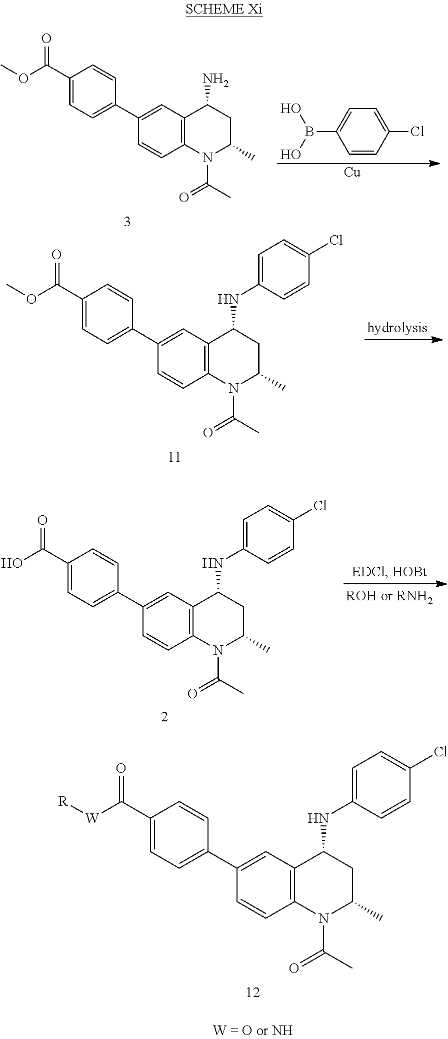

For example, R may be selected from the group consisting of:

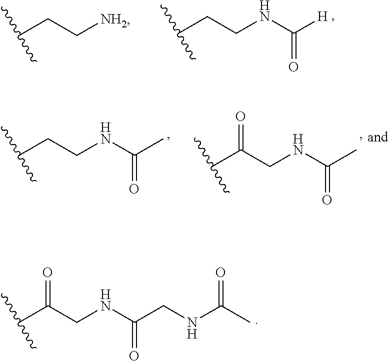

The synthetic route in Scheme Xj illustrates a general method for preparing tetrahydroquinoline-connector 1 derivatives having various connectors attached to both the tetrahydroquinoline compound and to any of the above-identified linkers (Z¹, Z², Z³ and Z⁴). In the scheme below, the linker moiety is designated by Z. The amino group of 4 is reacted with bromo-acetic acid to afford amide 13. The α-bromo amide 13 may be reacted with a variety of nucleophiles to afford tmahydroquinoline-connector 1 derivative 14, following deprotection of the benzylic amine. For example, Scheme Xj provides for a connector Y (e.g. Y₁, Y₂, Y₃ or Y₄) attached to a linker moiety (Z), wherein Y is e.g, —C(O)CH₂—R— of 14.

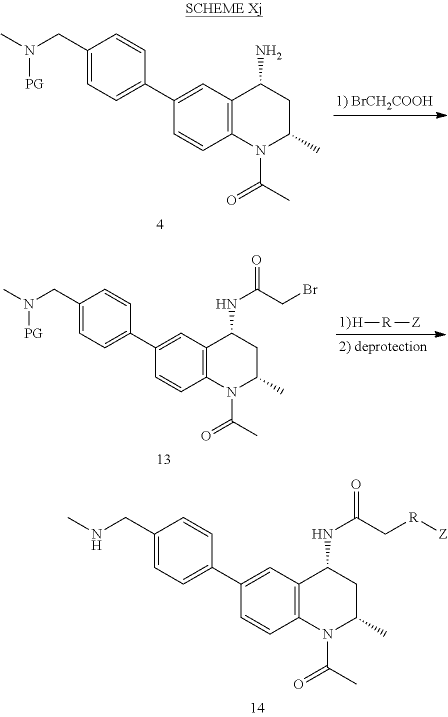

For example, R—Z may be selected from the group consisting of:

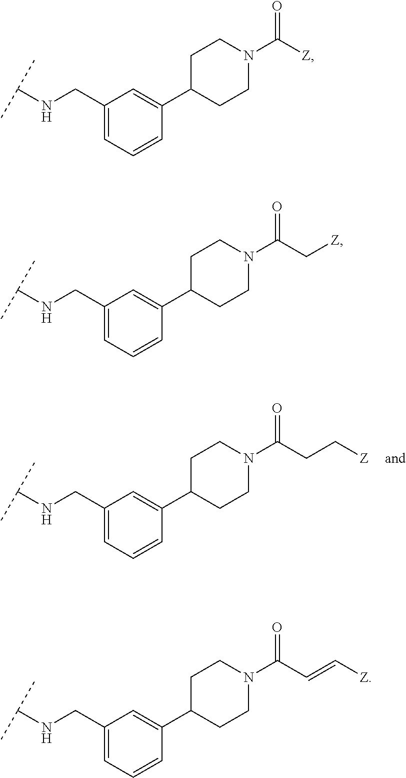

Additional examples for Z—R—H and —R—Z can be found in Table K, seen below:

TABLE K

| Example | | |
|---|---|---|
| No. | Z—R—H | —R—Z |
| 1 | 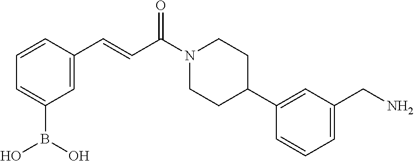 | 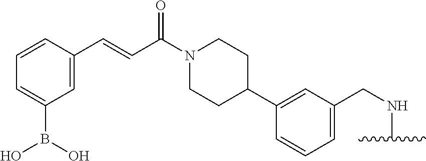 [SMILES: *NCc1cccc(C2CCN(C(=O)/C=C/c3cccc(B(O)O)c3)CC2)c1] |
| 2 | 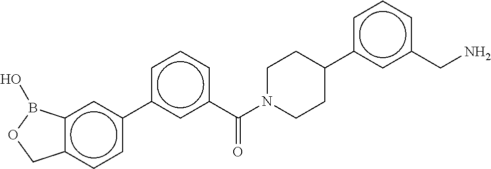 | 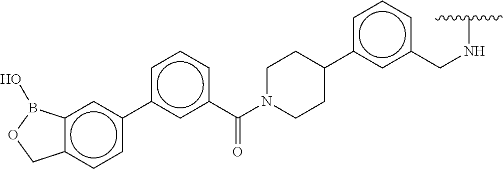 |
| 3 | 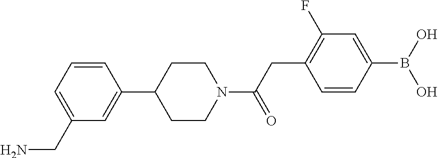 | 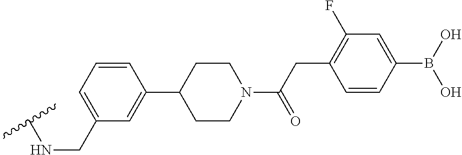 [SMILES: *NCc1cccc(C2CCN(C(=O)Cc3ccc(B(O)O)cc3F)CC2)c1] |
| 4 | 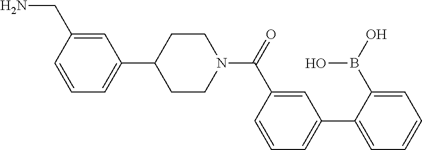 | 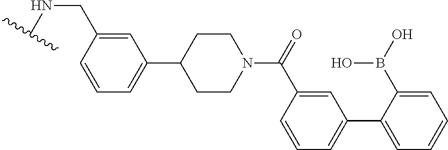 [SMILES: *NCc1cccc(C2CCN(C(=O)c3cccc(-c4ccccc4B(O)O)c3)CC2)c1] |
| 5 | 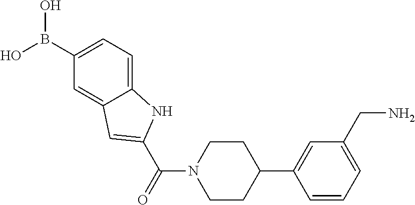 | 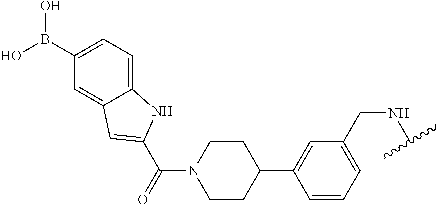 [SMILES: *NCc1cccc(C2CCN(C(=O)c3cc4cc(B(O)O)ccc4[nH]3)CC2)c1] |
| 6 | 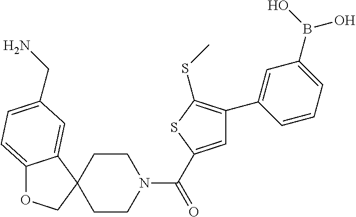 | 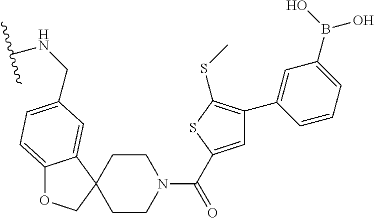 [SMILES: *NCc1ccc2c(c1)C1(CCN(C(=O)c3cc(-c4cccc(B(O)O)c4)c(SC)s3)CC1)CO2] |
| 7 | 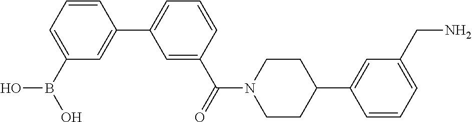 | 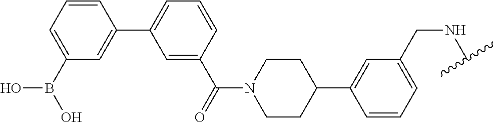 [SMILES: *NCc1cccc(C2CCN(C(=O)c3cccc(-c4cccc(B(O)O)c4)c3)CC2)c1] |
| 8 | 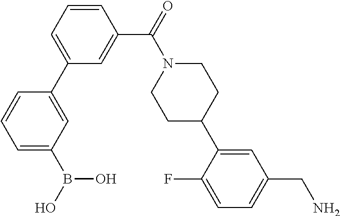 | 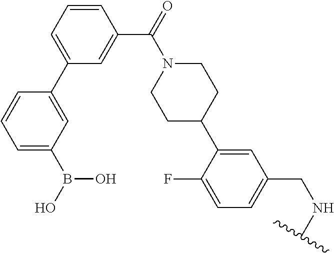 [SMILES: *NCc1ccc(F)c(C2CCN(C(=O)c3cccc(-c4cccc(B(O)O)c4)c3)CC2)c1] |
| 9 | 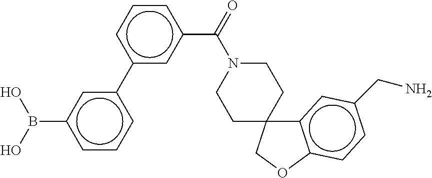 | 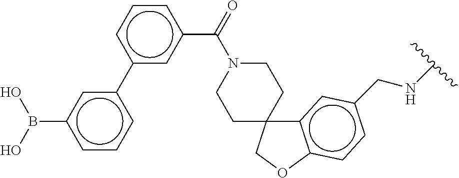 |
| 10 | 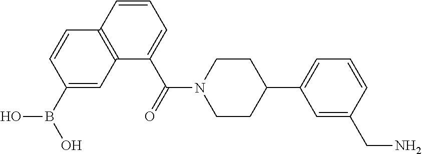 | 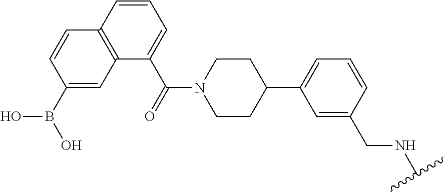 [SMILES: *NCc1cccc(C2CCN(C(=O)c3cccc4ccc(B(O)O)cc34)CC2)c1] |
| 11 | 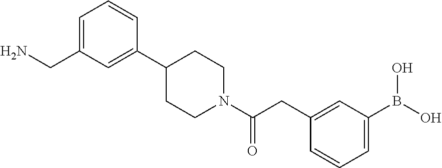 | 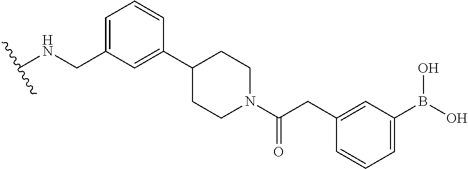 [SMILES: *NCc1cccc(C2CCN(C(=O)Cc3cccc(B(O)O)c3)CC2)c1] |
| 12 | 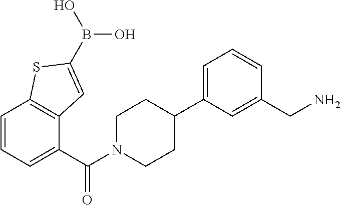 | 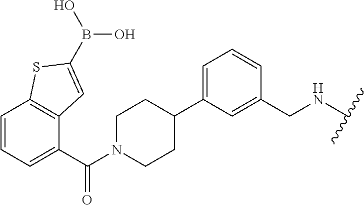 [SMILES: *NCc1cccc(C2CCN(C(=O)c3cccc4sc(B(O)O)cc34)CC2)c1] |
| 13 | 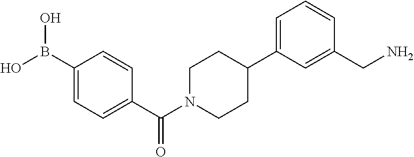 | 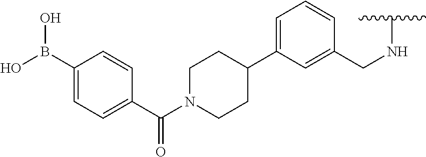 [SMILES: *NCc1cccc(C2CCN(C(=O)c3ccc(B(O)O)cc3)CC2)c1] |
| 14 | 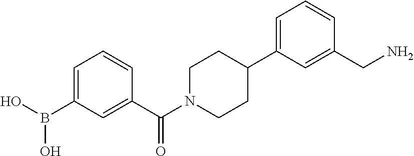 | 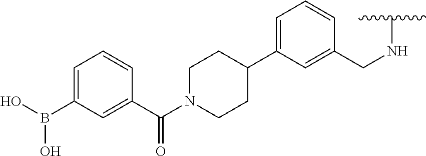 [SMILES: *NCc1cccc(C2CCN(C(=O)c3cccc(B(O)O)c3)CC2)c1] |
| 15 | 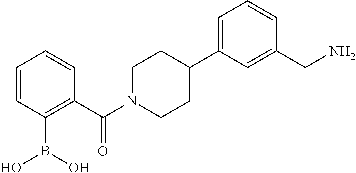 | 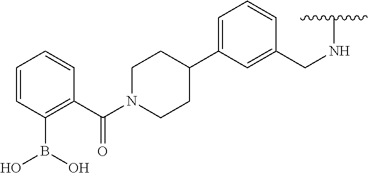 [SMILES: *NCc1cccc(C2CCN(C(=O)c3ccccc3B(O)O)CC2)c1] |
| 16 | 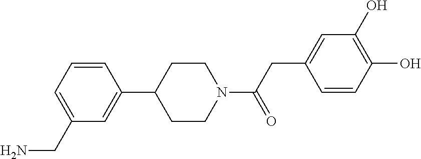 | 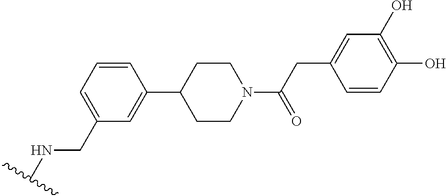 [SMILES: *NCc1cccc(C2CCN(C(=O)Cc3ccc(O)c(O)c3)CC2)c1] |
| 17 | 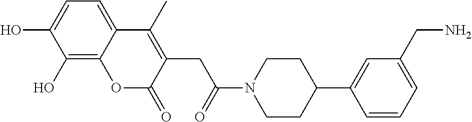 | 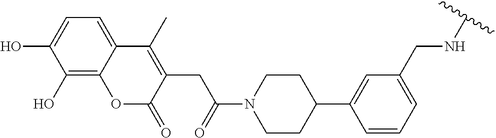 [SMILES: *NCc1cccc(C2CCN(C(=O)Cc3c(C)c4ccc(O)c(O)c4oc3=O)CC2)c1] |
| 18 | 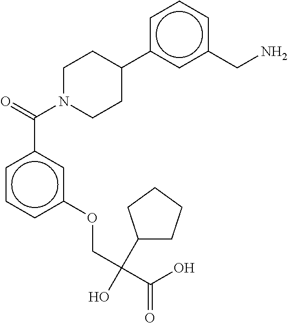 | 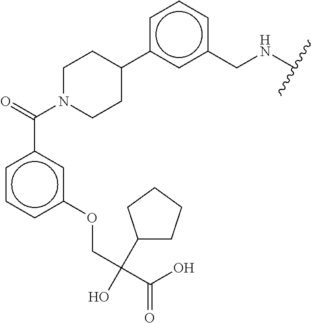 |
| 19 | 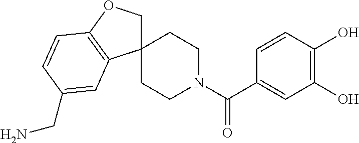 | 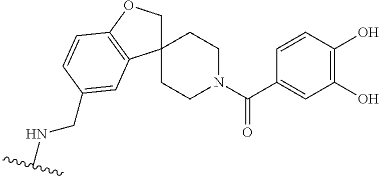 [SMILES: *NCc1ccc2c(c1)C1(CCN(C(=O)c3ccc(O)c(O)c3)CC1)CO2] |
| 20 | 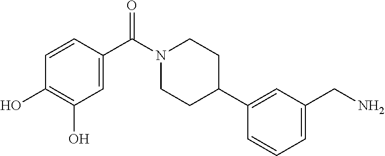 | 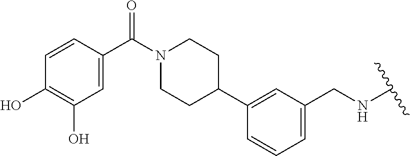 [SMILES: *NCc1cccc(C2CCN(C(=O)c3ccc(O)c(O)c3)CC2)c1] |
| 21 | 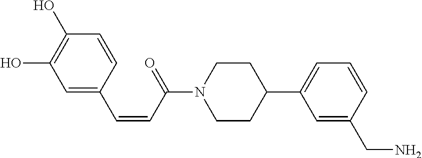 | 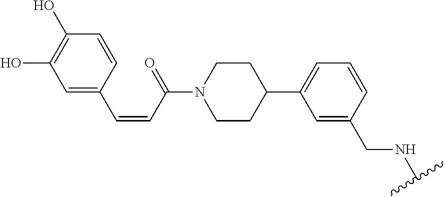 [SMILES: *NCc1cccc(C2CCN(C(=O)/C=C\c3ccc(O)c(O)c3)CC2)c1] |
| 22 | 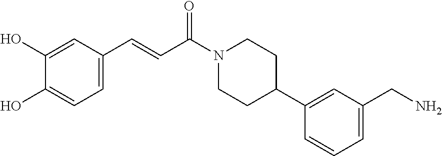 | 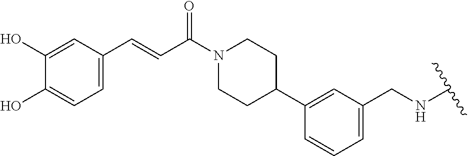 [SMILES: *NCc1cccc(C2CCN(C(=O)/C=C/c3ccc(O)c(O)c3)CC2)c1] |
| 23 | 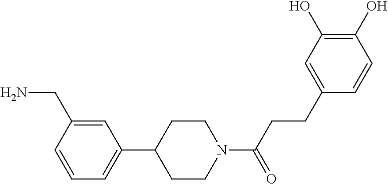 | 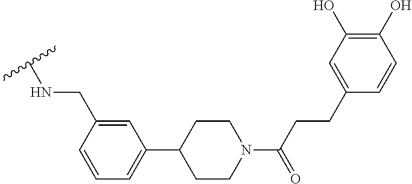 [SMILES: *NCc1cccc(C2CCN(C(=O)CCc3ccc(O)c(O)c3)CC2)c1] |
| 24 | 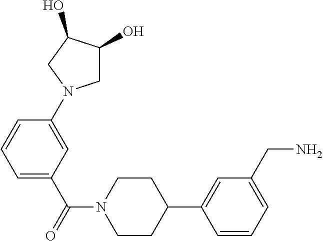 | 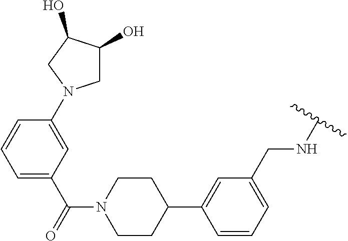 [SMILES: *NCc1cccc(C2CCN(C(=O)c3cccc(N4C[C@@H](O)[C@@H](O)C4)c3)CC2)c1] |
| 25 | 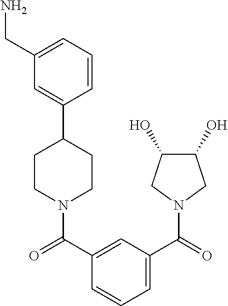 | 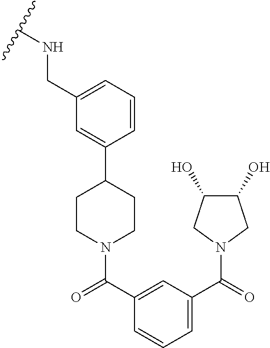 [SMILES: *NCc1cccc(C2CCN(C(=O)c3cccc(C(=O)N4C[C@@H](O)[C@@H](O)C4)c3)CC2)c1] |
| 26 | 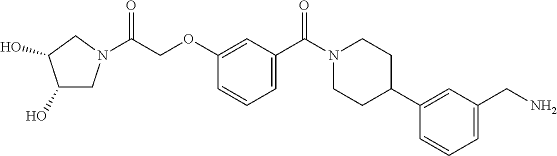 | 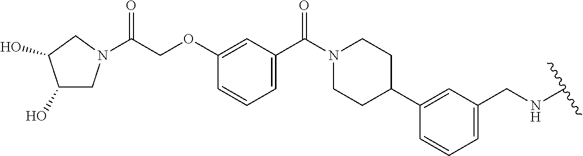 [SMILES: *NCc1cccc(C2CCN(C(=O)c3cccc(OCC(=O)N4C[C@@H](O)[C@@H](O)C4)c3)CC2)c1] |
| 27 | 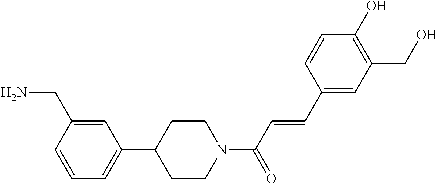 | 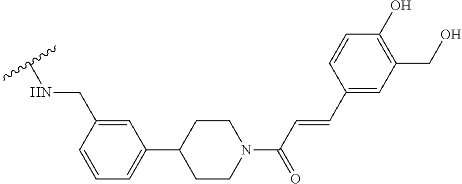 [SMILES: *NCc1cccc(C2CCN(C(=O)/C=C/c3ccc(O)c(CO)c3)CC2)c1] |
| 28 | 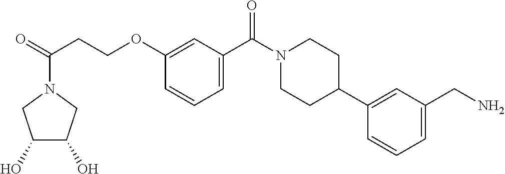 | 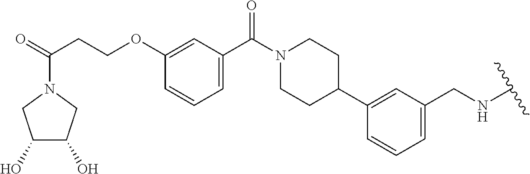 [SMILES: *NCc1cccc(C2CCN(C(=O)c3cccc(OCCC(=O)N4C[C@@H](O)[C@@H](O)C4)c3)CC2)c1] |
| 29 | 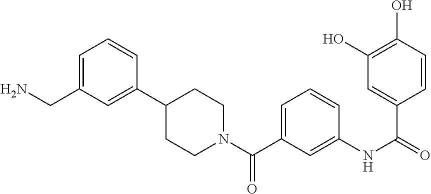 | 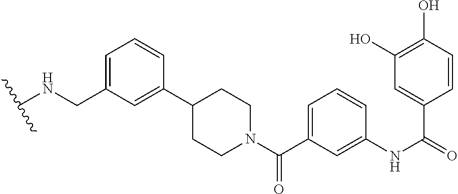 [SMILES: *NCc1cccc(C2CCN(C(=O)c3cccc(NC(=O)c4ccc(O)c(O)c4)c3)CC2)c1] |
| 30 | 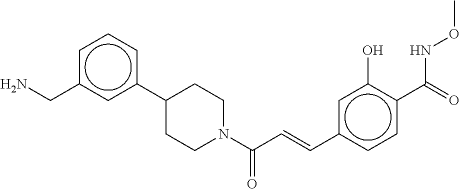 | 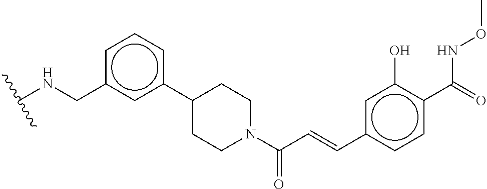 |
| 31 | 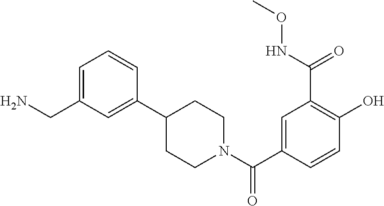 | 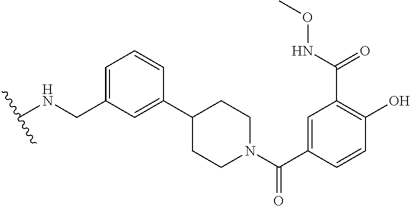 [SMILES: *NCc1cccc(C2CCN(C(=O)c3ccc(O)c(C(=O)NOC)c3)CC2)c1] |

The synthetic route in Scheme Xk illustrates a complementary method to Scheme Xj for preparing tetrahydroquinoline-connector 1 derivatives having various connectors attached to both the tetrahydroquinoline compound and to any of the above-identified linkers (Z¹, Z², Z³ and Z⁴). In the scheme below, the linker moiety is designated by Z. Unlike Scheme Xj, Scheme Xk provides a procedure for the direct linkage of a connector moiety to the carbonyl substituent. The amino group of 4 may be reacted with a variety of electrophiles, for example, a carboxylic acid, to afford tetrahydroquinoline-connector 1 derivative 15, following deprotection of the benzylic amine. For example, Scheme Xk provides for a connector Y (e.g. Y₁, Y₂, Y₃ or Y₄) attached to a linker moiety (Z), wherein Y is —R— (e.g., —R— of 15).

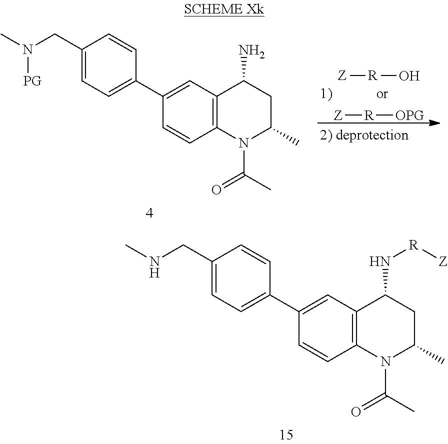

For example, R—Z may be represented by the structure:

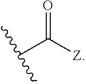

The synthetic route in Scheme Xl illustrates an method for preparing tetrahydroquinoline-connector 1 derivatives having various connectors attached to both the tetrahydroquinoline compound and to any of the above-identified linkers (Z¹, Z², Z³ and Z⁴). In the scheme below, the linker moiety is designated by Z. A portion of a connector moiety is installed via reaction of the amino group of 4 with acid 4a. Global deprotection of 16, affords the free amine of 16, which can be reacted with a variety of electrophiles, for example, a carboxylic acid, to afford tefrahydroquinoline-connector 1 derivative 17. For example, Scheme Xl provides for a connector Y (e.g. Y₁, Y₂, Y₃ or Y₄) attached to a linker moiety (Z), wherein Y is —C(O)CH₂NHR— (e.g., —C(O)CH₂NHR— of 17).

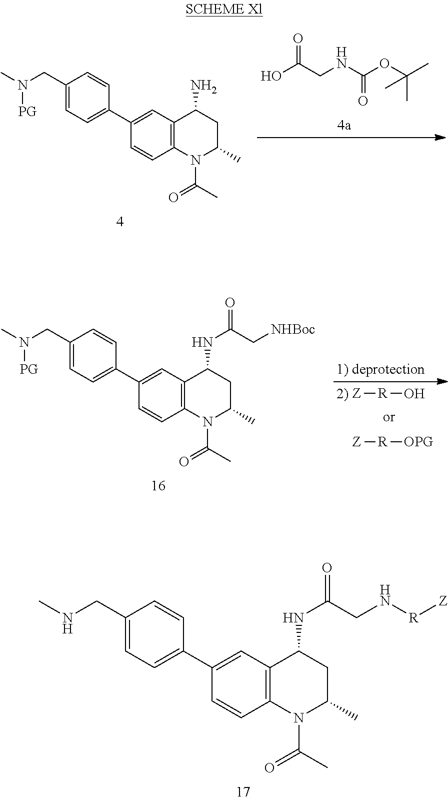

For example, R—Z may be represented by the structure:

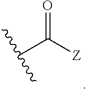

Additional examples for Z—R—OH and —R—Z that can be utilized in Scheme Xk and Scheme Xl can be found in Table L, seen below:

TABLE L

| Example No. | Z—R—OH or Z—R—OPG | —R—Z |
|---|---|---|
| 1 | 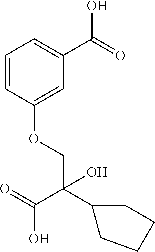 [SMILES: O=C(O)c1cccc(OCC(O)(C(=O)O)C2CCCC2)c1] | 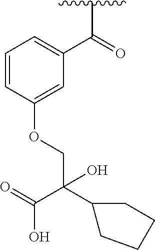 [SMILES: *C(=O)c1cccc(OCC(O)(C(=O)O)C2CCCC2)c1] |
| 2 | 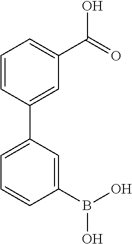 [SMILES: O=C(O)c1cccc(-c2cccc(B(O)O)c2)c1] | 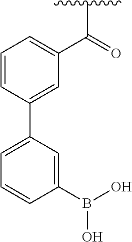 [SMILES: *C(=O)c1cccc(-c2cccc(B(O)O)c2)c1] |
| 3 | 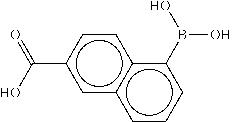 | 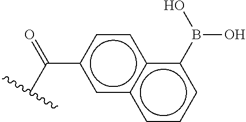 |
| 4 | 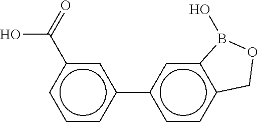 | 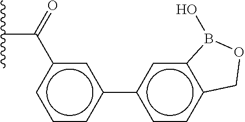 |
| 5 | 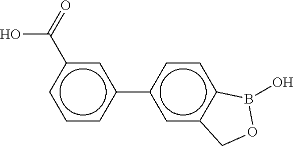 | 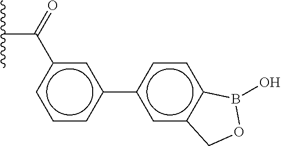 |
| 6 | 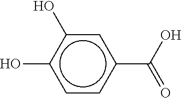 | 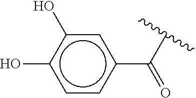 |
| 7 | 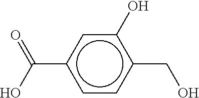 | 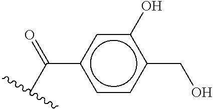 |
| 8 | 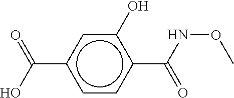 | 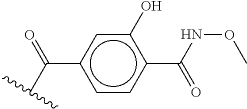 |
| 9 | 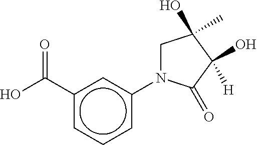 | 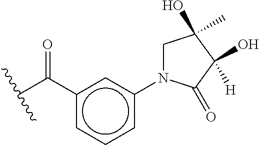 |
| 10 | 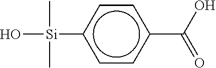 | 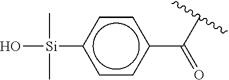 |
| 11 | 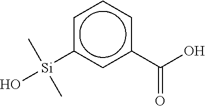 | 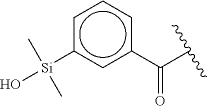 |
| 12 | 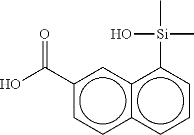 | 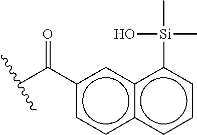 |
| 13 | 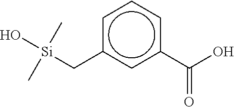 | 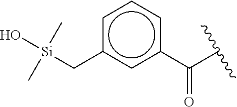 |
| 14 | 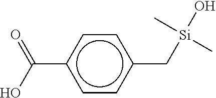 | 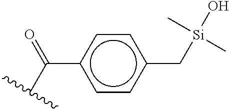 |
| 15 | 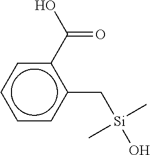 | 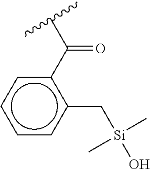 |
| 16 | 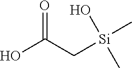 | 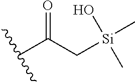 [SMILES: *C(=O)C[Si](C)(C)O] |

The above-identified imidazoquinoline compounds may have an attachment point for a connector element via the imidazole group. As seen below, a connector element may be identified as a Y group in imidazoquinoline-connector 1 C and imidazoquinoline-connector 1 D:

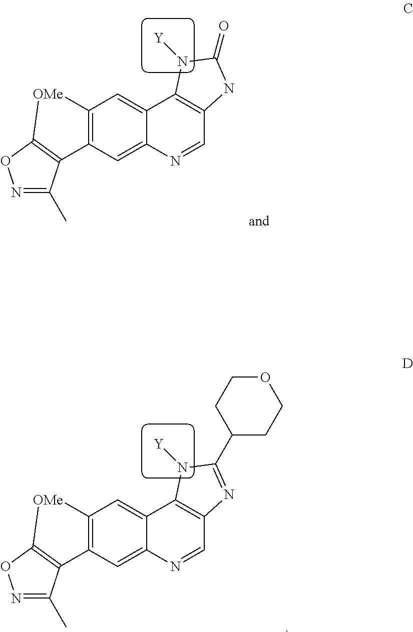

For example, Y¹, Y², Y³ and Y⁴ may be Y as described above in imidazoquinoline-connector 1 C or imidazoquinoline-connector 1 D.

The synthetic routes in Scheme Xm and Scheme Xn provide two complementary methods for preparing imidazoquinoline-connector 1 derivatives. In Scheme Xm, commercially available 6 is reacted with isoxazole 7 under Suzuki coupling conditions to prepare quinoline intermediate 8. The amine intermediate 9 is formed via nitration of quinoline 8 and is followed by chlorination to afford key intermediate 3. Nucleophilic aromatic substitution to install the desired Y group and reduction of the nitro group provides 10. In the final step, the fused imidazolidinone ring is is formed to afford 11 (imidazoquinoline-connector 1 derivative). For example, Scheme Xm provides for a connector Y (e.g. Y₁, Y₂, Y₃ or Y₄).

In Scheme Xn, commercially available diester 12 and aniline 13 are reacted to prepare the quinoline core intermediate 14. The isoxazole of 15 is installed via a Suzuki coupling. A three step procedure: hydrolysis, chlorination and amidation, provides carboxamide 4. Nucleophilic aromatic substitution is utilized to install the desired Y group, and formation of the imidazolidinone ring is the final step in the preparation of 18 (imidazoquinoline-connector 1 derivative). For example, Scheme Xn provides for a connector Y (e.g. Y₁, Y₂, Y₃ or Y₄).

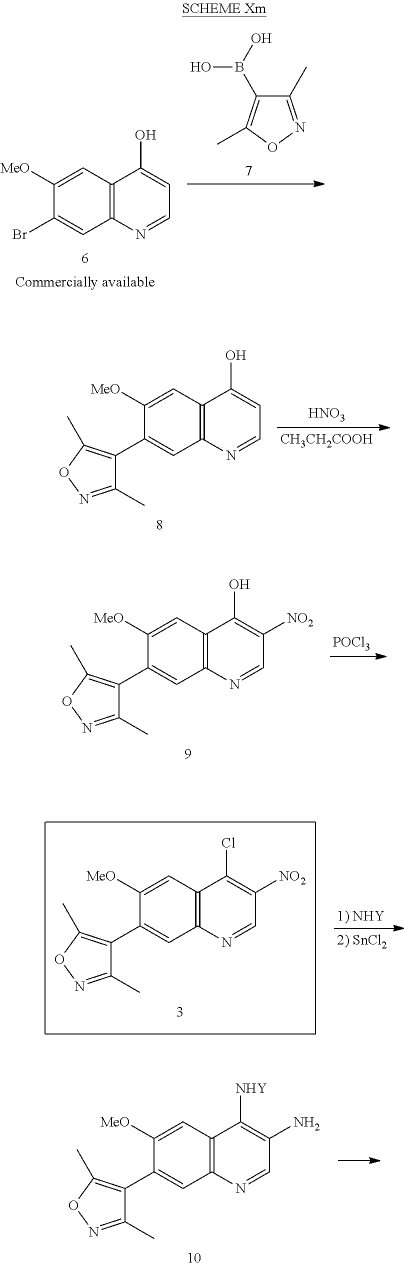

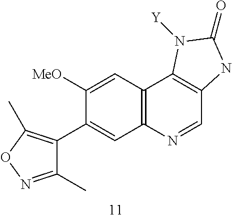

For example, Y may be selected from the group consisting of:

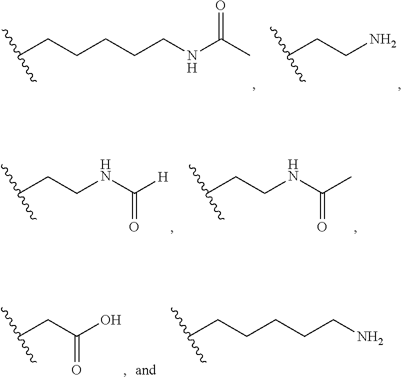

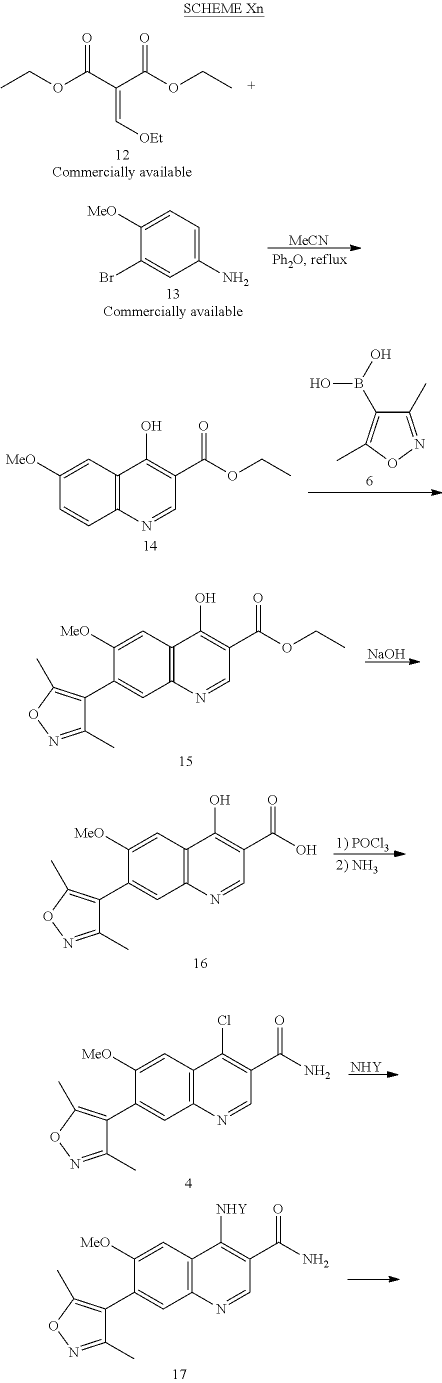

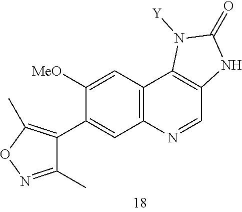

For example, Y may be selected from the group consisting of:

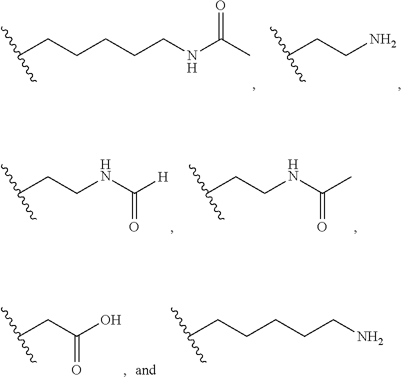

Additional examples for NHY and —Y that can be utilized in Scheme Xm and Scheme Xn can be found in Table M, seen below:

TABLE M

| Exam- | | |
|---|---|---|
| ple | | |
| No. | NH—Y | —Y |
| 1 | 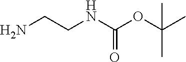 | 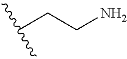 [SMILES: *CCN] |
| 2 | 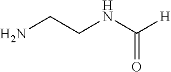 | 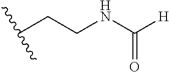 [SMILES: *CCNC=O] |

The divergent synthetic route in Scheme Xo illustrates a general method for providing imidazoquinoline-connector 1 derivatives having various connectors attached to both the imidazoquinoline compound and to any of the above-identified linkers (Z¹, Z², Z³ and Z⁴). In the scheme below, the linker moiety is designated by Z. Utilizing key intermediate 3 (synthesis described in Scheme Xm), nucleophilic aromatic substitution allows for the installation of the desired Y—Z group. The final divergent step is cyclization to provide imidazoquinoline 11 (fused-imidazoquinoline derivative C) and 21 (fused-imidazole derivative 0), respectively. For example, Scheme Xo provides for a connector Y (e.g. Y₁, Y₂, Y₃ or Y₄) attached to a linker moiety (Z).

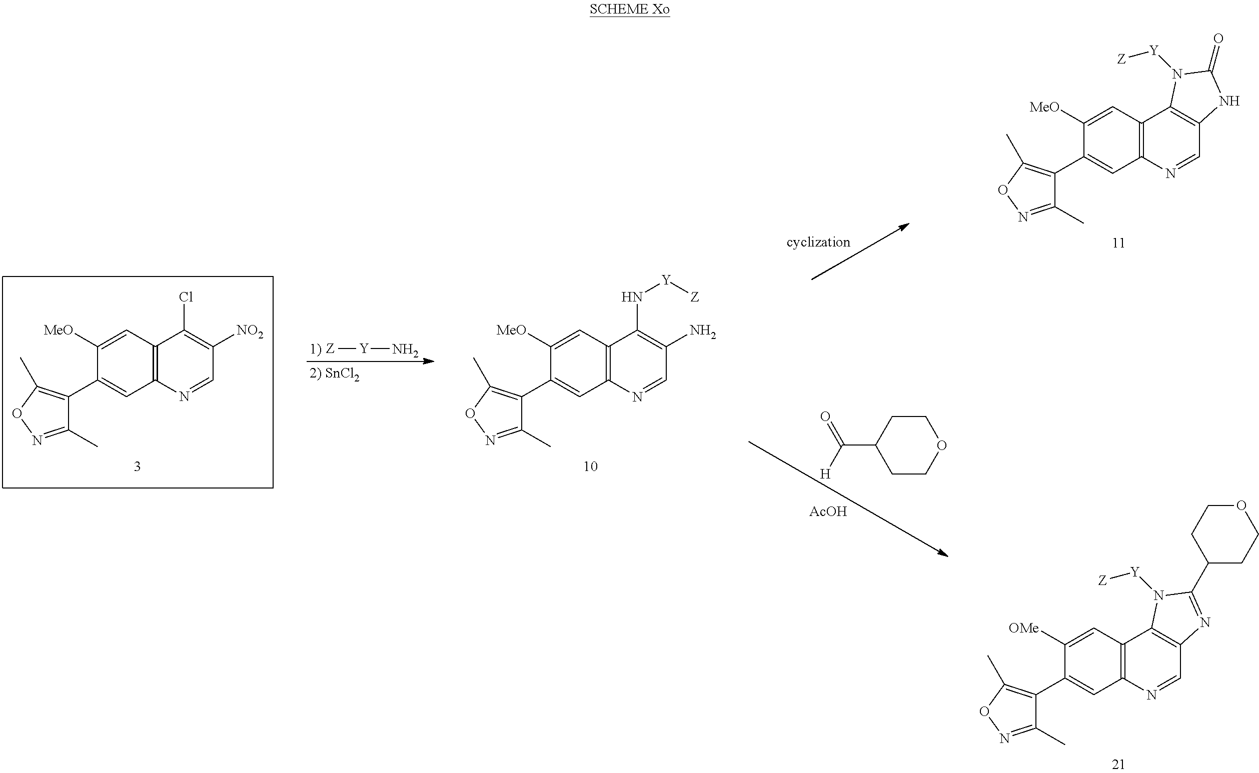

For example, —Y—Z may be selected from the group consisting of:

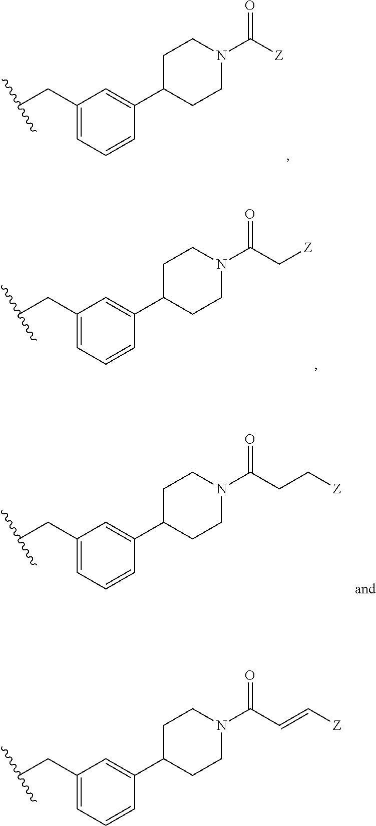

Additional examples for Z—Y—NH₂ and —Y—Z that can be utilized in Scheme Xo can be found in Table N, seen below:

TABLE N

| Ex- | | |
|---|---|---|
| am- | | |
| ple | | |
| No. | Z—Y—NH₂ | —Y—Z |
| 1 | 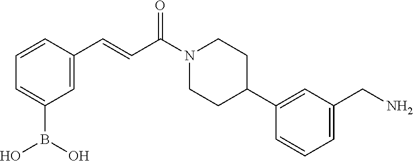 [SMILES: NCc1cccc(C2CCN(C(=O)/C=C/c3cccc(B(O)O)c3)CC2)c1] | 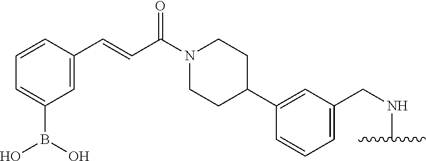 [SMILES: *NCc1cccc(C2CCN(C(=O)/C=C/c3cccc(B(O)O)c3)CC2)c1] |
| 2 | 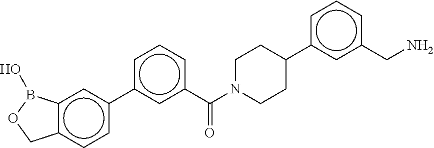 | 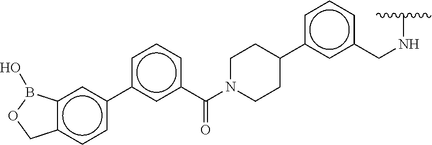 |
| 3 | 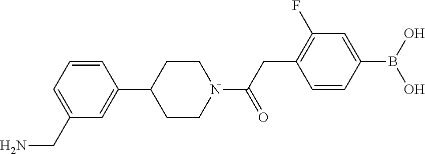 [SMILES: NCc1cccc(C2CCN(C(=O)Cc3ccc(B(O)O)cc3F)CC2)c1] | 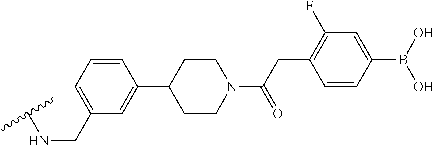 [SMILES: *NCc1cccc(C2CCN(C(=O)Cc3ccc(B(O)O)cc3F)CC2)c1] |
| 4 | 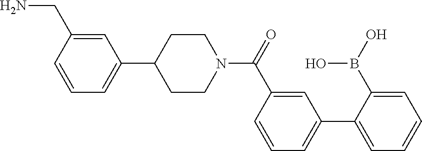 [SMILES: NCc1cccc(C2CCN(C(=O)c3cccc(-c4ccccc4B(O)O)c3)CC2)c1] | 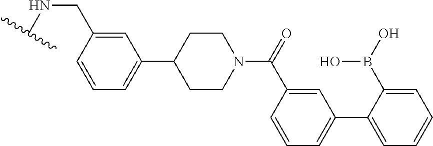 [SMILES: *NCc1cccc(C2CCN(C(=O)c3cccc(-c4ccccc4B(O)O)c3)CC2)c1] |
| 5 | 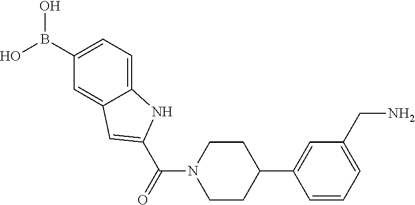 [SMILES: NCc1cccc(C2CCN(C(=O)c3cc4cc(B(O)O)ccc4[nH]3)CC2)c1] | 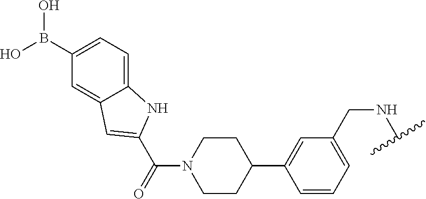 [SMILES: *NCc1cccc(C2CCN(C(=O)c3cc4cc(B(O)O)ccc4[nH]3)CC2)c1] |
| 6 | 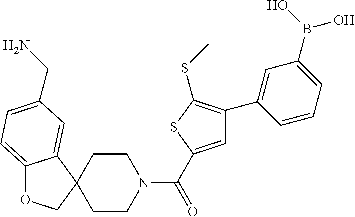 [SMILES: CSc1sc(C(=O)N2CCC3(CC2)COc2ccc(CN)cc23)cc1-c1cccc(B(O)O)c1] | 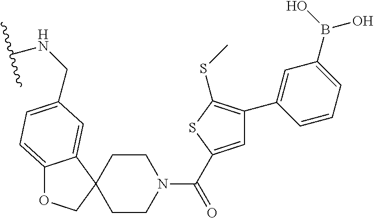 [SMILES: *NCc1ccc2c(c1)C1(CCN(C(=O)c3cc(-c4cccc(B(O)O)c4)c(SC)s3)CC1)CO2] |
| 7 | 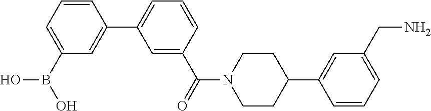 [SMILES: NCc1cccc(C2CCN(C(=O)c3cccc(-c4cccc(B(O)O)c4)c3)CC2)c1] | 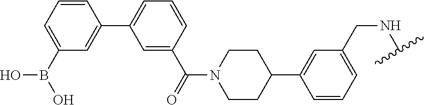 [SMILES: *NCc1cccc(C2CCN(C(=O)c3cccc(-c4cccc(B(O)O)c4)c3)CC2)c1] |
| 8 | 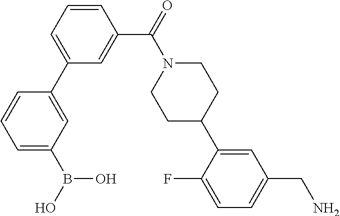 [SMILES: NCc1ccc(F)c(C2CCN(C(=O)c3cccc(-c4cccc(B(O)O)c4)c3)CC2)c1] | 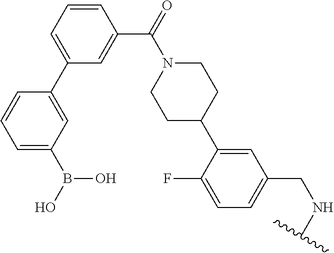 [SMILES: *NCc1ccc(F)c(C2CCN(C(=O)c3cccc(-c4cccc(B(O)O)c4)c3)CC2)c1] |
| 9 | 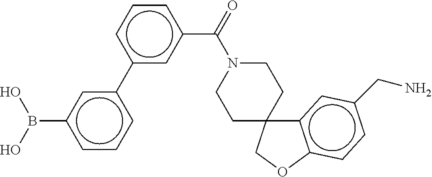 | 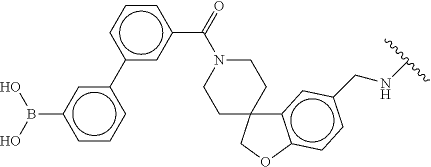 |
| 10 | 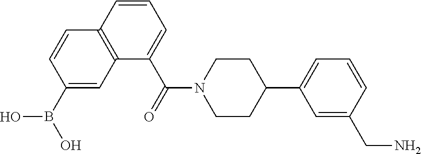 [SMILES: NCc1cccc(C2CCN(C(=O)c3cccc4ccc(B(O)O)cc34)CC2)c1] | 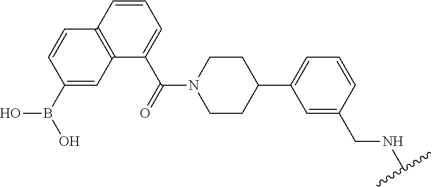 [SMILES: *NCc1cccc(C2CCN(C(=O)c3cccc4ccc(B(O)O)cc34)CC2)c1] |
| 11 | 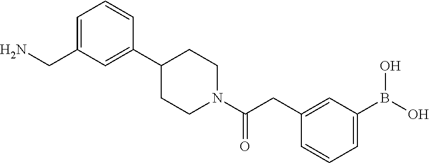 [SMILES: NCc1cccc(C2CCN(C(=O)Cc3cccc(B(O)O)c3)CC2)c1] | 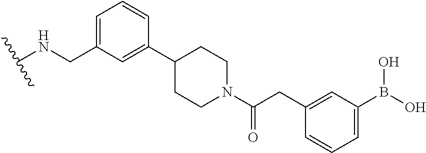 [SMILES: *NCc1cccc(C2CCN(C(=O)Cc3cccc(B(O)O)c3)CC2)c1] |
| 12 | 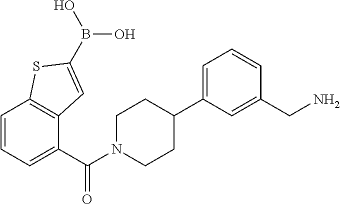 [SMILES: NCc1cccc(C2CCN(C(=O)c3cccc4sc(B(O)O)cc34)CC2)c1] | 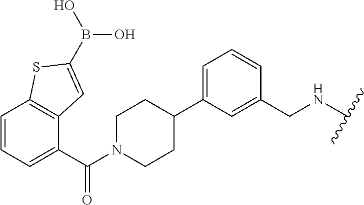 [SMILES: *NCc1cccc(C2CCN(C(=O)c3cccc4sc(B(O)O)cc34)CC2)c1] |
| 13 | 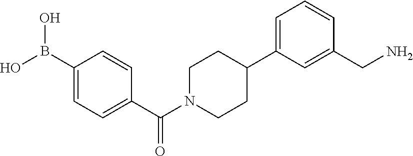 [SMILES: NCc1cccc(C2CCN(C(=O)c3ccc(B(O)O)cc3)CC2)c1] | 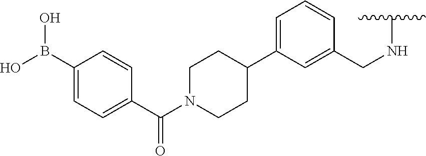 [SMILES: *NCc1cccc(C2CCN(C(=O)c3ccc(B(O)O)cc3)CC2)c1] |
| 14 | 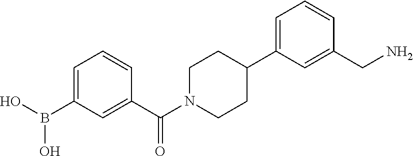 [SMILES: NCc1cccc(C2CCN(C(=O)c3cccc(B(O)O)c3)CC2)c1] | 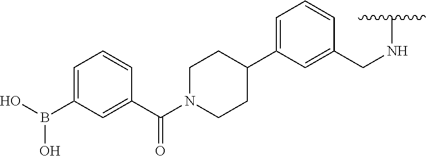 [SMILES: *NCc1cccc(C2CCN(C(=O)c3cccc(B(O)O)c3)CC2)c1] |
| 15 | 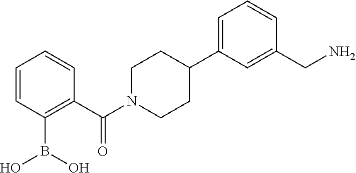 [SMILES: NCc1cccc(C2CCN(C(=O)c3ccccc3B(O)O)CC2)c1] | 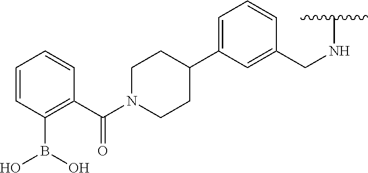 [SMILES: *NCc1cccc(C2CCN(C(=O)c3ccccc3B(O)O)CC2)c1] |
| 16 | 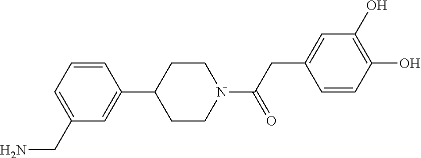 [SMILES: NCc1cccc(C2CCN(C(=O)Cc3ccc(O)c(O)c3)CC2)c1] | 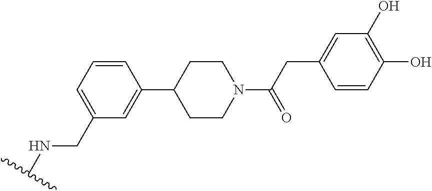 [SMILES: *NCc1cccc(C2CCN(C(=O)Cc3ccc(O)c(O)c3)CC2)c1] |
| 17 | 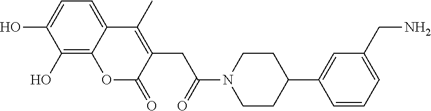 [SMILES: Cc1c(CC(=O)N2CCC(c3cccc(CN)c3)CC2)c(=O)oc2c(O)c(O)ccc12] | 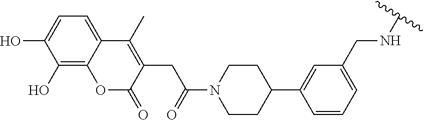 [SMILES: *NCc1cccc(C2CCN(C(=O)Cc3c(C)c4ccc(O)c(O)c4oc3=O)CC2)c1] |
| 18 | 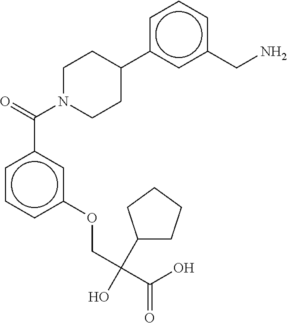 | 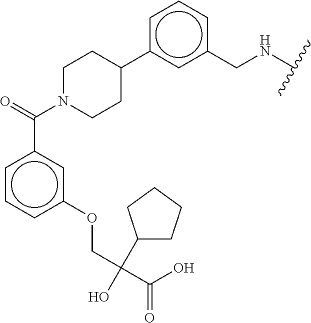 |
| 19 | 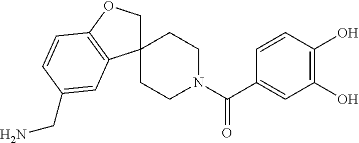 [SMILES: NCc1ccc2c(c1)C1(CCN(C(=O)c3ccc(O)c(O)c3)CC1)CO2] | 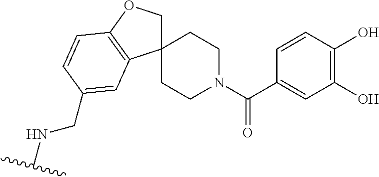 [SMILES: *NCc1ccc2c(c1)C1(CCN(C(=O)c3ccc(O)c(O)c3)CC1)CO2] |
| 20 | 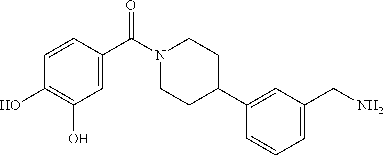 [SMILES: NCc1cccc(C2CCN(C(=O)c3ccc(O)c(O)c3)CC2)c1] | 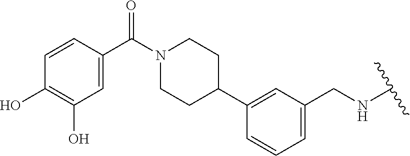 [SMILES: *NCc1cccc(C2CCN(C(=O)c3ccc(O)c(O)c3)CC2)c1] |
| 21 | 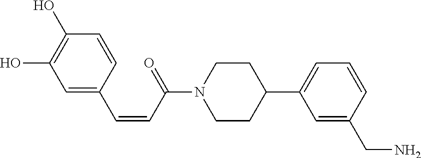 [SMILES: NCc1cccc(C2CCN(C(=O)/C=C\c3ccc(O)c(O)c3)CC2)c1] | 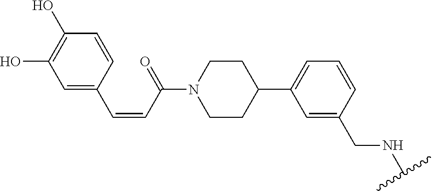 [SMILES: *NCc1cccc(C2CCN(C(=O)/C=C\c3ccc(O)c(O)c3)CC2)c1] |
| 22 | 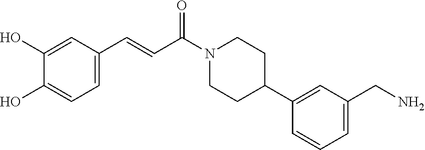 [SMILES: NCc1cccc(C2CCN(C(=O)/C=C/c3ccc(O)c(O)c3)CC2)c1] | 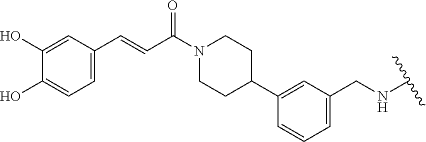 [SMILES: *NCc1cccc(C2CCN(C(=O)/C=C/c3ccc(O)c(O)c3)CC2)c1] |
| 23 | 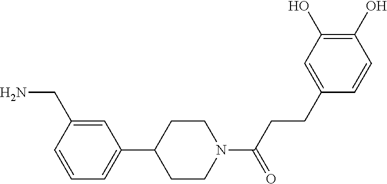 [SMILES: NCc1cccc(C2CCN(C(=O)CCc3ccc(O)c(O)c3)CC2)c1] | 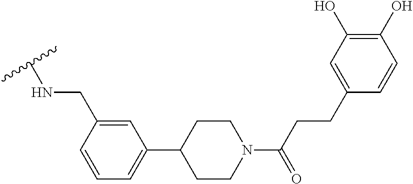 [SMILES: *NCc1cccc(C2CCN(C(=O)CCc3ccc(O)c(O)c3)CC2)c1] |
| 24 | 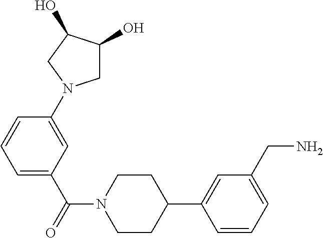 [SMILES: NCc1cccc(C2CCN(C(=O)c3cccc(N4C[C@@H](O)[C@@H](O)C4)c3)CC2)c1] | 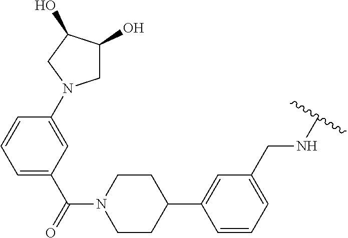 [SMILES: *NCc1cccc(C2CCN(C(=O)c3cccc(N4C[C@@H](O)[C@@H](O)C4)c3)CC2)c1] |
| 25 | 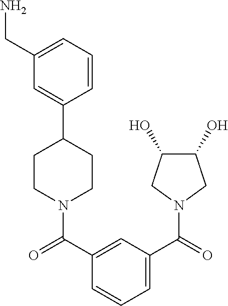 [SMILES: NCc1cccc(C2CCN(C(=O)c3cccc(C(=O)N4C[C@@H](O)[C@@H](O)C4)c3)CC2)c1] | 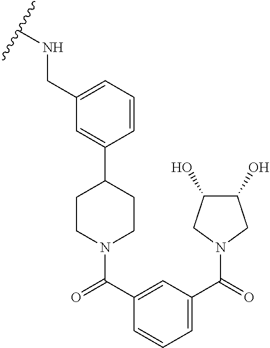 [SMILES: *NCc1cccc(C2CCN(C(=O)c3cccc(C(=O)N4C[C@@H](O)[C@@H](O)C4)c3)CC2)c1] |
| 26 | 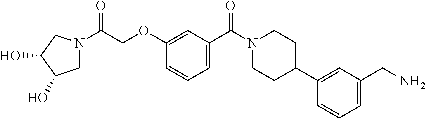 [SMILES: NCc1cccc(C2CCN(C(=O)c3cccc(OCC(=O)N4C[C@@H](O)[C@@H](O)C4)c3)CC2)c1] | 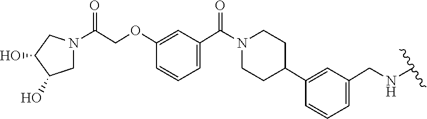 [SMILES: *NCc1cccc(C2CCN(C(=O)c3cccc(OCC(=O)N4C[C@@H](O)[C@@H](O)C4)c3)CC2)c1] |
| 27 | 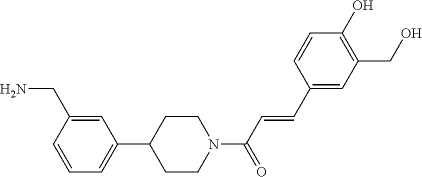 [SMILES: NCc1cccc(C2CCN(C(=O)/C=C/c3ccc(O)c(CO)c3)CC2)c1] | 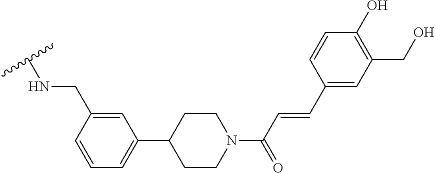 [SMILES: *NCc1cccc(C2CCN(C(=O)/C=C/c3ccc(O)c(CO)c3)CC2)c1] |
| 28 | 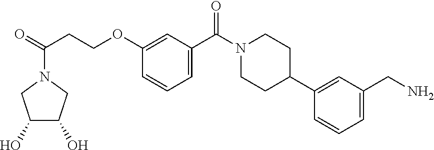 [SMILES: NCc1cccc(C2CCN(C(=O)c3cccc(OCCC(=O)N4C[C@@H](O)[C@@H](O)C4)c3)CC2)c1] | 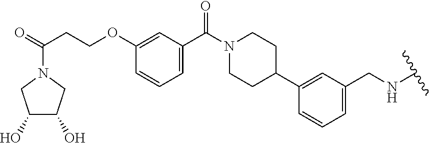 [SMILES: *NCc1cccc(C2CCN(C(=O)c3cccc(OCCC(=O)N4C[C@@H](O)[C@@H](O)C4)c3)CC2)c1] |
| 29 | 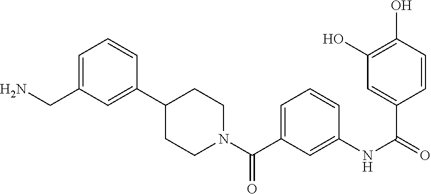 [SMILES: NCc1cccc(C2CCN(C(=O)c3cccc(NC(=O)c4ccc(O)c(O)c4)c3)CC2)c1] | 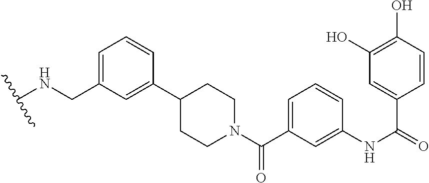 [SMILES: *NCc1cccc(C2CCN(C(=O)c3cccc(NC(=O)c4ccc(O)c(O)c4)c3)CC2)c1] |
| 30 | 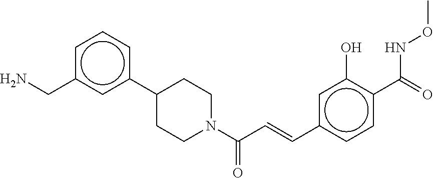 | 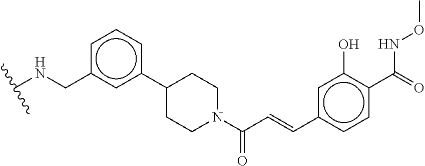 |
| 31 | 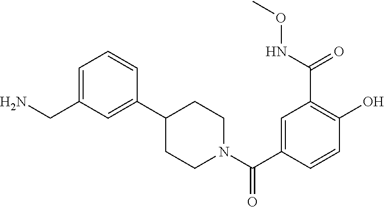 [SMILES: CONC(=O)c1cc(C(=O)N2CCC(c3cccc(CN)c3)CC2)ccc1O] | 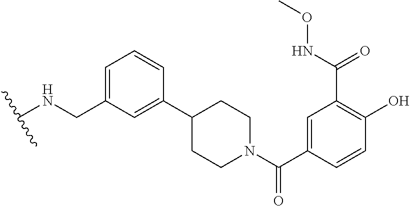 [SMILES: *NCc1cccc(C2CCN(C(=O)c3ccc(O)c(C(=O)NOC)c3)CC2)c1] |

The divergent synthetic route in Scheme Xq illustrates a general method for providing imidazoquinoline-connector 1 derivatives having various ethylene-substituted connectors attached to both the imidazoquinoline compound and to any of the above-identified linkers (Z¹, Z², Z³ and Z⁴). In the scheme below, the linker moiety is designated by Z. The ethylene diamine connector is installed via nucleophilic aromatic substitution. Following reduction of the nitro group to afford amino-quinoline 18, the divergent cyclization steps provide imidazoquinoline 19 (fused-imidazoquinoline) and 22 (fused-imidazole), respectively. The desired R—Z group is installed via reaction with a variety of electrophiles, for example, a carboxylic acid, to afford 20A (fused-imidazoquinoline derivative C) and 23 (fused-imidazole derivative D), respectively. For example, Scheme Xq provides for a connector Y (e.g. Y₁, Y₂, Y₃ or Y₄) attached to a linker moiety (Z), wherein Y is —CH₂CH₂NHR— (e.g., —CH₂CH₂NHR— of 20A or 23).

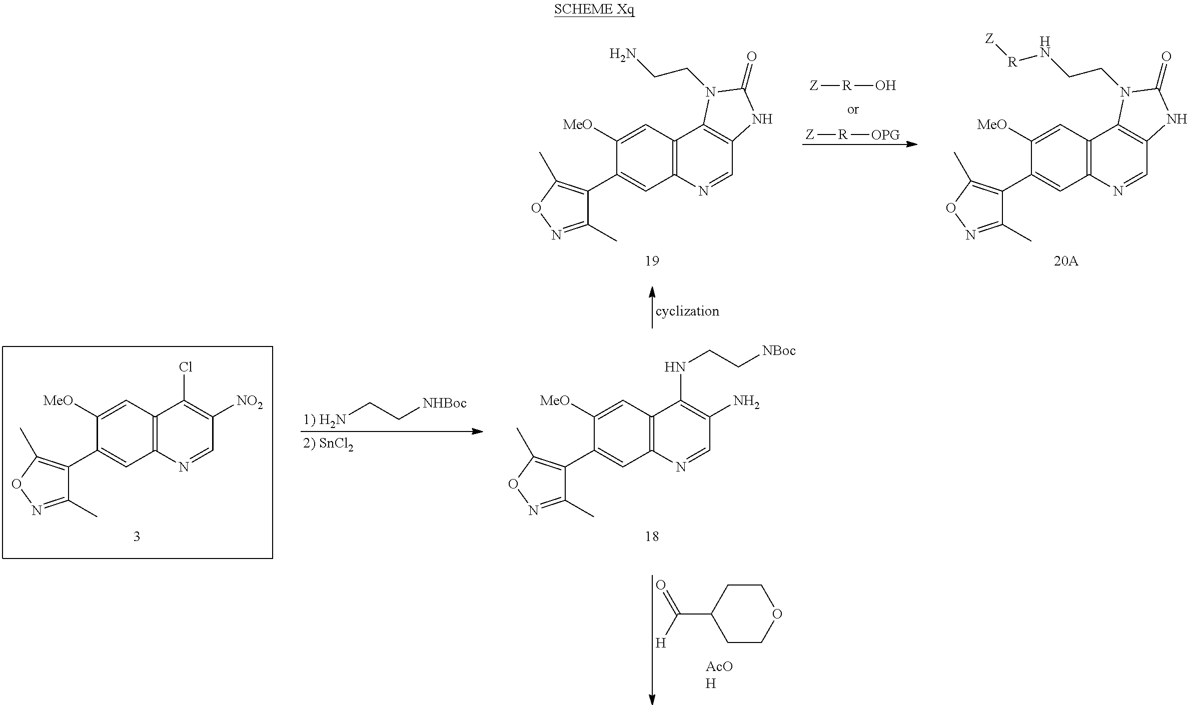

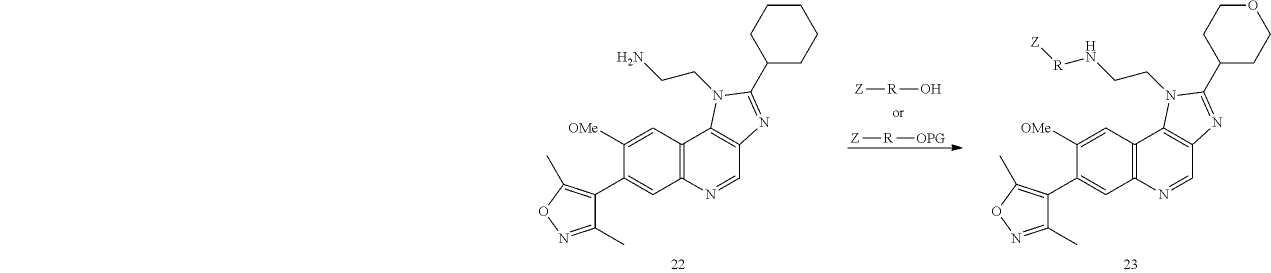

For example, R—Z may be represented by the structure:

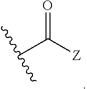

Additional examples for Z—R—OH and —R—Z that can be utilized in Scheme Xq can be found in Table O, seen below:

TABLE O

| Example No. | Z—R—OH or Z—R—OPG | —R—Z |
|---|---|---|
| 1 | 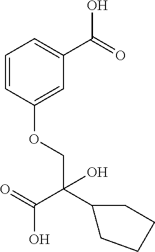 [SMILES: O=C(O)c1cccc(OCC(O)(C(=O)O)C2CCCC2)c1] | 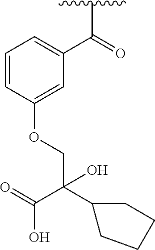 [SMILES: *C(=O)c1cccc(OCC(O)(C(=O)O)C2CCCC2)c1] |
| 2 | 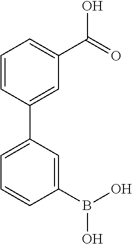 [SMILES: O=C(O)c1cccc(-c2cccc(B(O)O)c2)c1] | 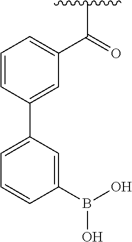 [SMILES: *C(=O)c1cccc(-c2cccc(B(O)O)c2)c1] |
| 3 | 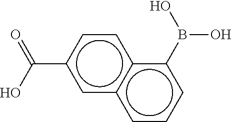 | 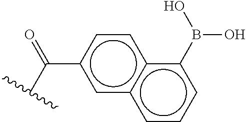 |
| 4 | 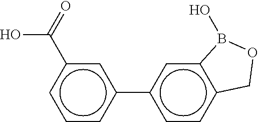 | 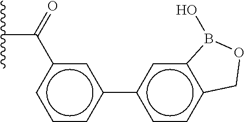 |
| 5 | 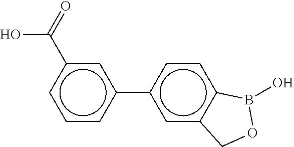 | 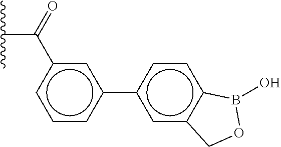 |
| 6 | 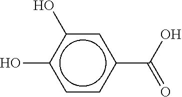 | 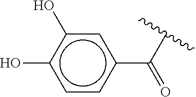 |
| 7 | 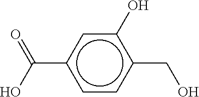 | 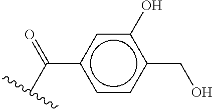 |
| 8 | 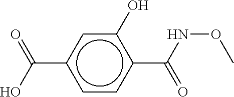 | 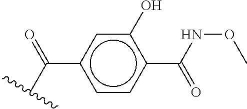 |
| 9 | 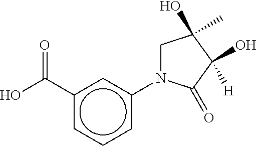 | 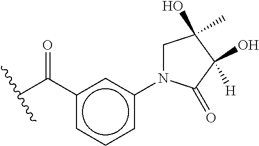 |
| 10 | 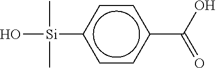 | 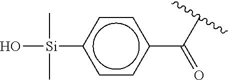 |
| 11 | 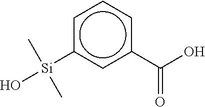 | 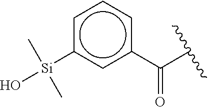 |
| 12 | 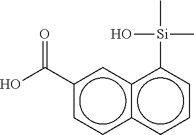 | 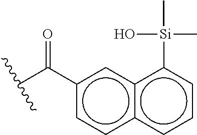 |
| 13 | 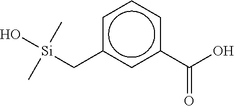 | 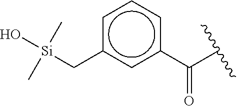 |
| 14 | 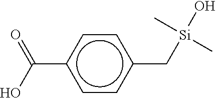 | 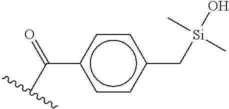 |
| 15 | 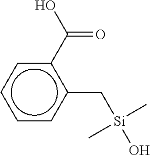 | 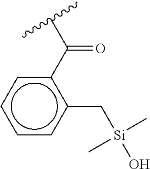 |
| 16 | 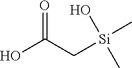 | 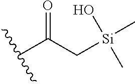 [SMILES: *C(=O)C[Si](C)(C)O] |

The above-identified isoxazole compounds may have one of e.g., two possible attachment points for a connector element: the phenyl ether and the benzylic ether. As seen below, a connector element may be identified as a Y group in isoxazole-connector 1 E and isoxazole-connector 2 F:

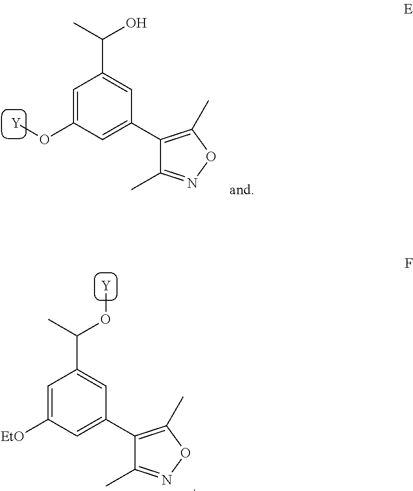

For example, Y¹, Y², Y³ and Y⁴ may be Y as described above in connector 1 E or connector 2 F.

The synthetic route in Scheme Xt illustrates a general method for preparing isoxazole-connector 1 derivatives. The method involves attaching the desired substituents to the phenol core. The desired Y group attached at the meta-position of the phenol can be installed by reacting isoxazole 1t with the appropriate electrophile 2 to provide 3t (isoxazole-connector 1 derivative). For example, Scheme Xt provides for a connector Y (e.g. Y¹, Y², Y³ or Y⁴).

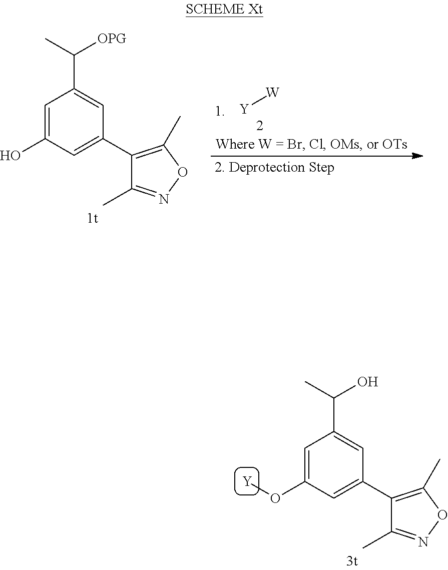

Similar to Scheme Xt, Scheme Xu provides a synthetic route for preparing isoxazole-connector 2 derivatives. The method involves attaching the desired substituents to the phenol core. The desired Y group attached at the benzylic alcohol can be installed by reacting isoxazole 1u with the appropriate electrophile 2 to provide 3u (isoxazole-connector 2 derivative). For example, Scheme Xu provides for a connector Y (e.g. Y¹, Y², Y³ or Y⁴).

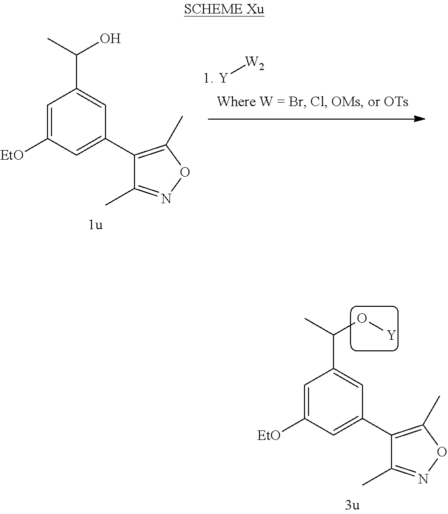

For Scheme Xt and Scheme Xu, additional examples for 2 and Y can be found in Table A.

In another embodiment, the attachment points identified in E (isoxazole-connector 1) or F (isoxazole-connector 2) may be further elaborated to incorporate not only a connector moiety (Y), but also a linker (Z), as e.g., represented by:

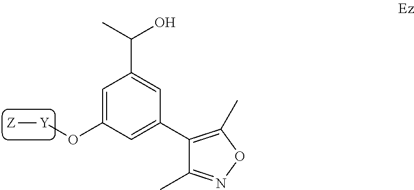

(isoxazole-connector 1) or

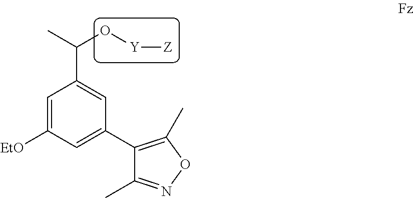

(isoxazole-connector 2). For example, Z (e.g., Z¹, Z², Z³ and Z⁴) may be any of the linker moieties contemplated herein.

The above-identified isoxazole compounds may connect to a connector through a different attachment point, e.g., the amino group of the quinazolone core. As seen below, a connector dement may identified e.g., as a Y group in isoxazole-connector 3 G:

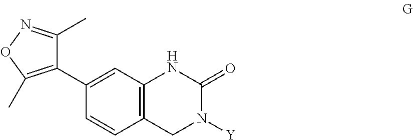

In one embodiment, the attachment point identified in G may be further elaborated to incorporate not only a connector moiety (Y), but also a linker (Z), as represented by

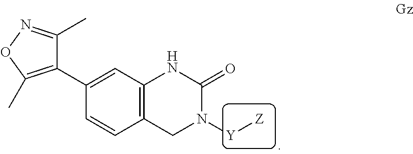

For example, Z (e.g., Z¹, Z², Z³ and Z⁴) may be any of the linker moieties contemplated herein.

Scheme Xv provides a synthetic procedure for making G derivatives having a connector attached to both the heterocyclic compound and to any of the above-identified linkers (Z¹, Z², Z³ and Z⁴). In the scheme below, the linker moiety is designated by Z. Starting from tri-substituted phenyl 1, the ethylene diamine substitutent (2) is attached via nucleophilic substitution. Reductive cyclization of 3 affords quinazolone 4. The isoxazole is installed utilizing a Suzuki coupling, and upon subsequent formation of 6, deprotection of the terminal amine provides 7. The desired R—Z group is installed via reaction with a variety of electrophiles, for example, a carboxylic acid, to afford 8 (isoxazole-conncetor 3 G). For example, Scheme Xv provides for a connector Y (e.g. Y₁, Y₂, Y₃ or Y₄) attached to a linker moiety (Z), wherein Y is —CH₂CH₂NHR— (e.g., —CH₂CH₂NHR— of 8).

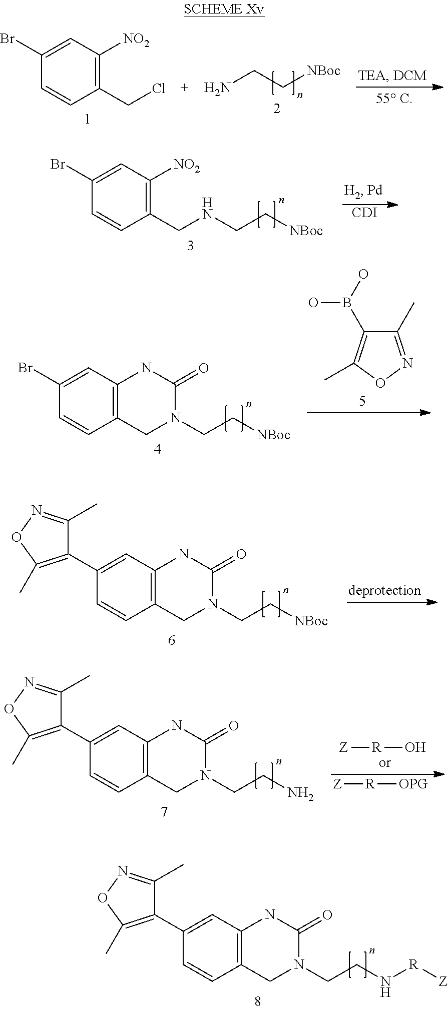

For example, R—Z may be represented by the structure:

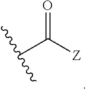

Additional examples for Z—R—OH or Z—R—OPG and —R—Z that can be utilized in Scheme Xv can be found in Table P, seen below:

TABLE P

| Example No. | Z—R—OH or Z—R—OPG | —R—Z |
|---|---|---|
| 1 | 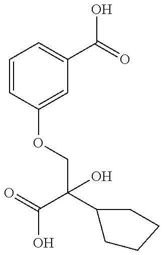 [SMILES: O=C(O)c1cccc(OCC(O)(C(=O)O)C2CCCC2)c1] | 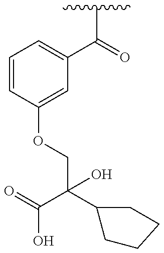 [SMILES: *C(=O)c1cccc(OCC(O)(C(=O)O)C2CCCC2)c1] |
| 2 | 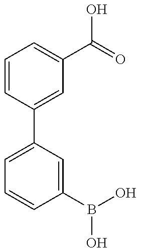 [SMILES: O=C(O)c1cccc(-c2cccc(B(O)O)c2)c1] | 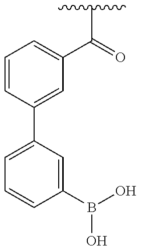 [SMILES: *C(=O)c1cccc(-c2cccc(B(O)O)c2)c1] |
| 3 | 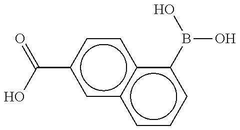 | 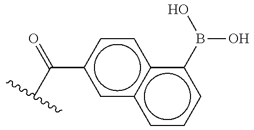 |
| 4 | 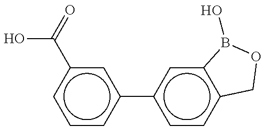 | 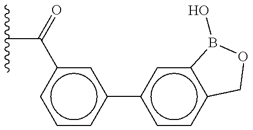 |
| 5 | 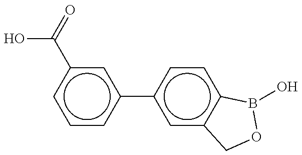 | 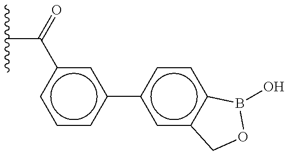 |
| 6 | 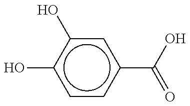 | 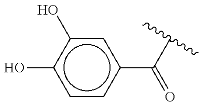 |
| 7 | 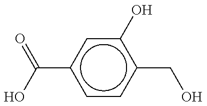 | 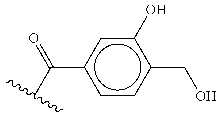 |
| 8 | 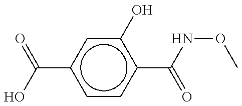 | 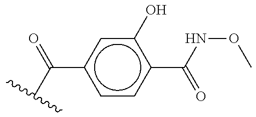 |
| 9 | 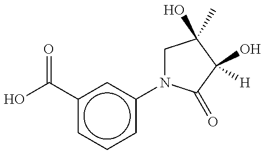 | 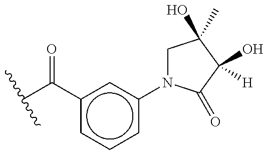 |
| 10 | 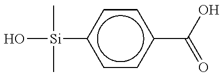 | 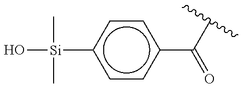 |
| 11 | 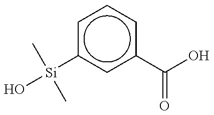 | 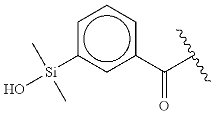 |
| 12 | 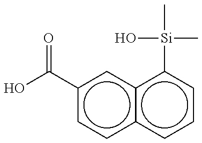 | 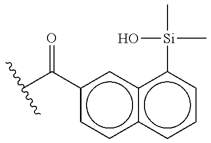 |
| 13 | 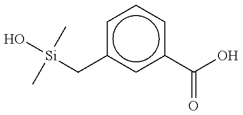 | 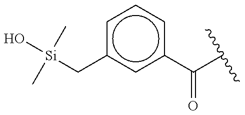 |
| 14 | 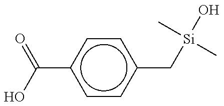 | 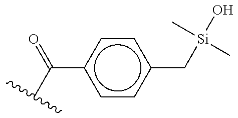 |
| 15 | 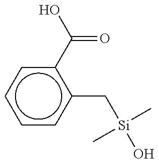 | 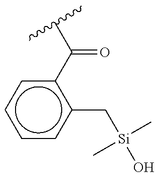 |
| 16 | 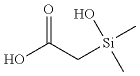 | 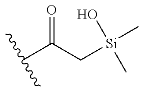 [SMILES: *C(=O)C[Si](C)(C)O] |

### Multimers

In some embodiments, a first monomer and a second monomer may form a dimer in aqueous solution. For example, in some instances, the first monomer may form a biologically useful dimer with a second monomer in vivo.

Without wishing to be bound by any theory, it is believed that molecular self-assembly may be directed through noncovalent interactions, e.g., hydrogen bonding, metal coordination, hydrophobic forces, van der Waals forces, pi-pi interactions, electrostatic, and/or electromagnetic interactions.

Without wishing to be bound by any theory, pi-pi and pi-cation interactions can be used to drive multimerization. In addition, van der Waals and electromagnetic forces are other interactions that can help to drive multimerization. Alternatively, acid/base pairs and donor-acceptor pairs, e.g., amide and/or sulfonamide pairs, can be employed to help direct self-assembly. In other cases, use of hydrophobic interactions can be used for multimerization targeting a membrane-bound protein. Additionally, metal coordination might be used when the target itself incorporates the metal, but could also be used in other scenarios.

In one embodiment, a therapeutic multimer compound may be formed from the multimerization in an aqueous media of a first monomer X¹—Y¹—Z¹ with a second monomer X²—Y²—Z². For example, Z¹ is a first linker capable of binding to the second monomer, wherein Z² is a second linker capable of binding to the first monomer through Z¹. In a certain embodiment, Z² is a nucleophile moiety capable of binding with the Z¹ moiety of Formula I to form the multimer. In another embodiment, the first monomer forms a biologically useful dimer with a second monomer in vivo.

In another embodiment, a therapeutic multimer compound may be formed from the multimerization in an aqueous media of a first monomer X¹—Y¹—Z¹ with a second monomer X⁴—Y⁴—Z⁴. For example, Z¹ is a first linker capable of binding to the second monomer, wherein Z⁴ is a second linker capable of binding to the first monomer through Z¹.

In certain embodiments, the multimerization may be substantially irreversible in an aqueous media. For example, the multimerization with Formula Is may be photolytically induced. In another example, Z¹ may be independently selected for each occurrence from the group consisting of Formula Ia, Ia′, Ib, Ic, Id, Ie, Ie′ and Ih and Z² may be independently selected for each occurrence from the group consisting of Formula Im, In, Io, Ip, Ir and Is; and wherein N₂ may be released during the multimerization. In some instances, the multimer may be fluorescent.

It is contemplated herein that while many chemistries are in principle reversible, the extent, probability and rate of the reverse reaction will depend heavily upon a range of conditions including temperature, concentration, solvent, catalysis, and binding to the target biomolecule. The term “irreversible” typically refers to the low probability of the reverse reaction occurring to a significant extent in an aqueous media within the timeframe of associated biological, pharmacologic and metabolic events, e.g., turn-over or degradation of the target biomolecule, signal transduction responses, drug metabolism and clearance, etc. As the affinity of the “irreversible” multimeric assembly for the target biomolecule is at least an order of magnitude higher than that of its monomers, it is likely to persist on the target for a prolonged period and exhibit a very slow off-rate. Additionally, the binding of the “irreversible” multimeric assembly by the target biomolecule may also significantly slow the dissociative reversal of the linker reaction to regenerate monomers. Also, the irreversible extrusion of a small molecule from the multimer linkage, may ensure the linker reaction cannot be revered in an aqueous or biological milieu. Thus, in general the half-life for the “irreversible” multimeric assembly is considered e.g., comparable to, or longer than the half-life for, the associated biological processes, with the potential to provide a relatively long duration of pharmacologic action.

In some embodiments, X¹ and X² may be the same. In other cases, X¹ and X² may be different. In some embodiments, X¹ and X⁴ may be the same. In other cases, X¹ and X⁴ may be different.

In another embodiment, a first monomer, a second monomer and bridge monomer may be capable of forming a biologically useful multimer. The biologically useful multimer having at least three segments when the first monomer is in contact with the bridge monomer and when the bridge monomer is in contact with the second monomer in an aqueous media, wherein the first monomer is represented by:
X¹—Y¹—Z¹ (Formula I) and pharmaceutically acceptable salts, stereoisomers, metabolites and hydrates thereof, wherein X¹ is a first ligand moiety; Y¹ is absent or is a connector moiety covalently bound to X¹ and Z¹; Z¹ is a first linker capable of binding to the bridge monomer; the bridge monomer is represented by:
W¹—Y³—W² (Formula III), wherein W¹ is a second linker capable of binding to the first monomer through Z¹; Y³ is absent or is a connector moiety covalently bound to W¹ and W²; W² is a third linker capable of binding to the second monomer; and the second monomer is represented by:
X²—Y²—Z² (Formula II) wherein X² is a second ligand moiety; Y² is absent or is a connector moiety covalently bound to X² and Z²; Z² is a fourth linker capable of binding to the bride monomer through W²; and

wherein upon contact with the aqueous composition, said first monomer, second monomer and bridge monomer forms a multimer that binds to a target biomolecule.

### Methods

In some embodiments, contemplated monomers and multimers may be administered to a patient in need thereof. In some embodiments, a method of administering a pharmaceutically effective amount of a multimeric compound to a patient in need thereof is provided. In some cases, the method comprises administering to the patient thereof an amount of the first monomer and an amount of a second monomer in amounts effective such that the pharmaceutically effective amount of the resulting multimer is formed in vivo.

In some embodiments, a first monomer and a second monomer may be administered substantially sequentially. In other embodiments, the first monomer and the second monomer are administered substantially simultaneously. In some embodiments the monomers may be administered, sequentially or simultaneously, by different routes of administration or the same route of administration. In still further embodiments, a first monomer and a second monomer may be administered after forming a multimer.

In some instances, a method of modulating two or more target biomolecule domains is provided, e.g., two bromodomains. In some embodiments, a first ligand moiety (e.g., bound to a first monomer) may bind to a first bromodomain and a second ligand moiety (e.g., bound to a second monomer) may bind to a second domain. In certain embodiments, a multimer comprising the first and second ligand moieties may form prior to binding the first and second domains. In other embodiments, a multimer may form after one and/or two of the monomers bind the first and second domains.

In some embodiments, a multimer contemplated herein may be used to inhibit or facilitate protein-protein interactions. For example, in some cases, a contemplated multimer may be capable of activating or inactivating a signaling pathway. Without wishing to be bound by any theory, a multimer may bind to a target protein and affect the conformation of the target protein such that the target protein is more biologically active as compared to when the multimer does not bind the target protein. In some embodiments monomers may be chosen such that a multimer formed from the monomers binds to at least two regions of a target molecule.

In one embodiment, a contemplated multimer may be capable of binding to a bromodomain and a second protein domain, wherein the second protein domain is, e.g. between about 5 \acute{Å} and about 30 \acute{Å} of the bromodomain, or in some embodiments within about 40 \acute{Å} of the bromodomain.

In one embodiment, compounds contemplated herein may be capable of modulating oncology fusion proteins. For example, a multimer may be capable of modulating oncology fusion proteins. Methods of modulating oncology fusion proteins include methods of modulating, e.g., BRD-NUT. In some embodiments, the oncology fusion protein (e.g., fusion gene product) is a BRD fusion product, for example, BRD3-NUT and BRD4-NUT. For example, a method of modulating a fusion protein provided, wherein the fusion protein is selected from the group consisting of BRD3-NUT and BRD4-NUT.

In an embodiment, the compounds contemplated herein may be used in a method for treating diseases or conditions for which a bromodomain inhibitor is indicated, for example, a compound may be used for treating a chronic autoimmune and/or inflammatory condition in a patient in need thereof. In another embodiment, the compounds contemplated herein may be used in a method for treating cancer, such as midline carcinoma. For example, provided herein is a method of treating a disease associated with a protein having tandem bromodomains in a patient in need.

Provided herein, for example, is a use of a compound in the manufacture of a medicament for the treatment of diseases or conditions for which a bromodomain inhibitor is indicated. In another embodiment, provided herein is a use of a compound or a pharmaceutically acceptable salt thereof in the manufacture of a medicament for the treatment of a chronic autoimmune and/or inflammatory condition. In a further embodiment, provided herein is a use of a compound or a pharmaceutically acceptable salt thereof in the manufacture of a medicament for the treatment of cancer, such as midline carcinoma or acute myeloid leukemia.

Provided herein is a method of treating a disease or condition such as systemic or tissue inflammation, inflammatory responses to infection or hypoxia, cellular activation and proliferation, lipid metabolism, fibrosis, or the prevention and treatment of viral infections in a patient in need thereof comprising administering a pharmaceutically effective amount of two or more disclosed monomers, e.g. simultaneously or sequentially, or administering a contemplated multimer.

For example, methods of treating chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), asthma, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, depression, retinitis, uveitis, scleritis, hepatitis, pancreatitis, primary biliary cirrhosis, sclerosing cholangitis, Addison's disease, hypophysitis, thyroiditis, type II diabetes, acute rejection of transplanted organs in a patient in need thereof are contemplated, comprising administering two or more disclosed monomers, e.g. capable of forming a multimer, e.g., dimer in-vivo, or administering a contemplated multimer.

Also contemplated herein are methods of treating acute inflammatory conditions in a patient in need thereof such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, or vasculitis with organ involvement, comprising administering administering two or more disclosed monomers, e.g. capable of forming a multimer e.g., dimer in-vivo.

Methods of treating disorders relating to inflammatory responses to infections with bacteria, viruses, fungi, parasites or their toxins, in a patient in need thereof is contemplated, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria, SIRS associated with viral infections such as influenza, herpes zoster, herpes simplex, coronavirus, cold sores, chickenpox, shingles, human papilloma virus, cervical neoplasia, adenovirus infections, including acute respiratory disease, poxvirus infections such as cowpox and smallpox and African swine fever virus comprising administering administering two or more disclosed monomers, e.g. capable of forming a multimer e.g., dimer in-vivo, or administering a contemplated multimer.

Contemplated monomers or multimers may be useful, when administered to a patient in need thereof, in the prevention or treatment of conditions associated with ischaemia-reperfusion injury in a patient need thereof such as myocardial infarction, cerebrovascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

Other contemplated methods of treatment that include administering disclosed compounds include treatment of disorders of lipid metabolism via the regulation of APO-A1 such as hypercholesterolemia, atherosclerosis and Alzheimer's disease, treatment of fibrotic conditions such as idiopathic pulmonary fibrosis, renal fibrosis, post-operative stricture, keloid formation, scleroderma, cardiac fibrosis, and the prevention and treatment of viral infections such as herpes virus, human papilloma virus, adenovirus and poxvirus and other DNA viruses.

Contemplated herein are methods of treating cancers, e.g., cancers such as including hematological, epithelial including lung, breast and colon carcinomas, mesenchymal, hepatic, renal and neurological tumors, comprising administering a disclosed compound to a patient in need thereof. For example, contemplated herein is a method of treating squamous cell carcinoma, midline carcinoma or leukemia such as acute myeloid leukemia in a patient in need thereof comprising administering two or more disclosed monomers such that the monomers form a multimer (e.g. dimer) in-vivo.

In an embodiment, two or more contemplated monomers that e.g., form a multimer in-vivo, or a contemplated multimer, may be administered at the point of diagnosis to reduce the incidence of: SIRS, the onset of shock, multi-organ dysfunction syndrome, which includes the onset of acute lung injury, ARDS, acute renal, hepatic, and cardiac and gastro-intestinal injury.

Also contemplated herein are methods of providing contraceptive agents, or a method of providing contraception, to a male patient, comprising administering two or more disclosed monomers, or a disclosed multimer.

In some embodiments, a ligand moiety (e.g., a pharmacophore) may have a molecular weight between 50 Da and 2000 Da, in some embodiments between 50 Da and 1500 Da, in some embodiments, between 50 Da and 1000 Da, and in some embodiments, between 50 Da and 500 Da. In certain embodiments, a ligand moiety may have a molecular weight of less than 2000 Da, in some embodiments, less than 1000 Da, and in some embodiments less than 500 Da.

In certain embodiments, the compound utilized by one or more of the foregoing methods is one of the generic, subgeneric, or specific compounds described herein.

Disclosed compositions may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound may be administered orally, subcutaneously, topically, parenterally, by inhalation spray or rectally in dosage unit formulations containing conventional non-toxic pharmaceutically acceptable carriers, adjuvants, and vehicles. Parenteral administration may include subcutaneous injections, intravenous or intramuscular injections, or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a partial or total alleviation of symptoms, is achieved.

In another aspect, pharmaceutical compositions comprising monomers, dimers, and/or multimers as disclosed herein formulated together with a pharmaceutically acceptable carrier provided. In particular, the present disclosure provides pharmaceutical compositions comprising monomers, dimers, and/or multimers as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, enteral pharmaceutical formulations including a disclosed pharmaceutical composition comprising monomers, dimers, and/or multimers, an enteric material; and a pharmaceutically acceptable carrier or excipient thereof are provided. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleat, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit 5100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that may be used.

Advantageously, kits are provided containing one or more compositions each including the same or different monomers. Such kits include a suitable dosage form such as those described above and instructions describing the method of using such dosage form to treat a disease or condition. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent, or administering a second active agent.

Certain terms employed in the specification, examples, and appended claims are collected here. These definitions should be read in light of the entirety of the disclosure and understood as by a person of skill in the art. Unless defined otherwise, all technical and scientific terms used herein have the same meaning as commonly understood by a person of ordinary skill in the art.

### DEFINITIONS

In some embodiments, the compounds, as described herein, may be substituted with any number of substituents or functional moieties. In general, the term “substituted” whether preceded by the term “optionally” or not, and substituents contained in formulas, refer to the replacement of hydrogen radicals in a given structure with the radical of a specified substituent.

In some instances, when more than one position in any given structure may be substituted with more than one substituent selected from a specified group, the substituent may be either the same or different at every position.

As used herein, the term “substituted” is contemplated to include all permissible substituents of organic and inorganic compounds. In a broad aspect, the permissible substituents include acyclic and cyclic, branched and unbranched, carbocyclic and heterocyclic, aromatic and non-aromatic substituents of organic compounds. In some embodiments, heteroatoms such as nitrogen may have hydrogen substituents and/or any permissible substituents of organic compounds described herein which satisfy the valencies of the heteroatoms. Non-limiting examples of substituents include acyl; aliphatic; heteroaliphatic; phenyl; naphthyl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —SCN; —SRₓ; —CF₃; —CH₂CF₃; —CHCl₂; —CH₂OH; —CH₂CH₂OH; —CH₂NH₂; —CH₂SO₂CH₃; —ORₓ, —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —SORₓ; —S(O)₂Rₓ; —NRₓC(O)Rₓ; or —C(Rₓ)₃; wherein each occurrence of Rₓ independently is hydrogen, aliphatic, heteroaliphatic, phenyl, naphthyl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the phenyl, naphthyl, or heteroaryl substituents described above and herein may be substituted or unsubstituted. Furthermore, the compounds described herein are not intended to be limited in any manner by the permissible substituents of organic compounds. In some embodiments, combinations of substituents and variables described herein may be preferably those that result in the formation of stable compounds. The term “stable,” as used herein, refers to compounds which possess stability sufficient to allow manufacture and which maintain the integrity of the compound for a sufficient period of time to be detected and preferably for a sufficient period of time to be useful for the purposes detailed herein.

The term “acyl,” as used herein, refers to a moiety that includes a carbonyl group. In some embodiments, an acyl group may have a general formula selected from —C(O)Rₓ; —CO₂(Rₓ); —C(O)N(Rₓ)₂; —OC(O)Rₓ; —OCO₂Rₓ; and —OC(O)N(Rₓ)₂; wherein each occurrence of Rₓ independently includes, but is not limited to, hydrogen, aliphatic, heteroaliphatic, phenyl, naphthyl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the phenyl, naphthyl, or heteroaryl substituents described above and herein may be substituted or unsubstituted.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc.

In general, the terms “aryl,” “aromatic,” “heteroaryl,” and “heteroaromtic” as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In certain embodiments, aryl or aromatic refers to a mono- or bicyclic carbocyclic ring system having one or two aromatic rings selected from phenyl, naphthyl, tetrahydronaphthyl, indanyl, and indenyl. In certain embodiments, the term heteroaryl, as used herein, refers to a cyclic aromatic radical having from five to ten ring atoms of which one ring atom is selected from the group consisting of S, O, and N; zero, one, or two ring atoms are additional heteroatoms independently selected from the group consisting of S, O, and N; and the remaining ring atoms are carbon, the radical being joined to the rest of the molecule via any of the ring atoms. Heteroaryl moieties may be selected from: pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl, aromatic, heteroaryl, and heteroaromatic groups described herein can be unsubstituted or substituted, wherein substitution includes replacement of one, two, three, or more of the hydrogen atoms thereon independently with a group selected from: C₁₋₆alkyl; phenyl; heteroaryl; benzyl; heteroarylalkyl; C₁₋₆alkoxy; C₁₋₆cycloalkoxy; C₁₋₆heterocyclylalkoxy; C₁₋₆heterocyclyloxy; heterocyclyloxyalkyl; C₂₋₆alkenyloxy; C₂₋₆alkynyloxy; phenoxy; heteroalkoxy; heteroaryloxy; C₁₋₆alkylthio; phenylthio; heteroalkylthio; heteroarylthio; oxo; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ is selected from hydrogen, C₁₋₆alkyl, aliphatic, heteroaliphatic, phenyl, or heteroaryl. Additional examples of generally applicable substituents are illustrated by the specific embodiments shown in the Examples that are described herein.

The term “heterocyclic,” as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from the group consisting of oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from the group consisting of O, S, and N (wherein the nitrogen and sulfur heteroatoms may be optionally oxidized), including, but not limited to, a bi- or tri-cyclic group, comprising fused six-membered rings having between one and three heteroatoms independently selected from the group consisting of the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term “alkenyl” as used herein refers to an unsaturated straight or branched hydrocarbon having at least one carbon-carbon double bond, such as a straight or branched group of 2-6 or 3-4 carbon atoms, referred to herein for example as C₂₋₆alkenyl, and C₃₋₄alkenyl, respectively. Exemplary alkenyl groups include, but are not limited to, vinyl, allyl, butenyl, pentenyl, etc.

The term “alkenyloxy” used herein refers to a straight or branched alkenyl group attached to an oxygen (alkenyl-O). Exemplary alkenoxy groups include, but are not limited to, groups with an alkenyl group of 3-6 carbon atoms referred to herein as C₃₋₆alkenyloxy. Exemplary “alkenyloxy” groups include, but are not limited to allyloxy, butenyloxy, etc.

The term “alkoxy” as used herein refers to a straight or branched alkyl group attached to an oxygen (alkyl-O—). Exemplary alkoxy groups include, but are not limited to, groups with an alkyl group of 1-6 or 2-6 carbon atoms, referred to herein as C₁₋₆alkoxy, and C₂-C₆alkoxy, respectively. Exemplary alkoxy groups include, but are not limited to methoxy, ethoxy, isopropoxy, etc.

The term “alkoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkynyloxy” used herein refers to a straight or branched alkynyl group attached to an oxygen (alkynyl-O)). Exemplary alkynyloxy groups include, but are not limited to, propynyloxy.

The term “alkyl” as used herein refers to a saturated straight or branched hydrocarbon, for example, such as a straight or branched group of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylene” as used herein refers to a bivalent saturated straight or branched hydrocarbon, for example, such as a straight or branched group of 1-6, 1-4, or 1-3 carbon atoms, referred to herein as —C₁₋₆alkylene-, —C₁₋₄alkylene-, and —C₁₋₃alkylene-, respectively, where the alkylene has two open valences. Exemplary alkyl groups include, but are not limited to, methylene, ethylene, propylene, isopropylene, 2-methyl-1-propylene, 2-methyl-2-propylene, 2-methyl-1-butylene, 3-methyl-1-butylene, 3-methyl-2-butylene, 2,2-dimethyl-1-propylene, 2-methyl-1-pentylene, 3-methyl-1-pentylene, 4-methyl-1-pentylene, 2-methyl-2-pentylene, 3-methyl-2-pentylene, 4-methyl-2-pentylene, 2,2-dimethyl-1-butylene, 3,3-dimethyl-1-butylene, 2-ethyl-1-butylene, butylene, isobutylene, t-butylene, pentylene, isopentylene, neopentylene, hexylene, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond, such as a straight or branched group of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “carboxylic acid” as used herein refers to a group of formula —CO₂H.

The term “cyano” as used herein refers to the radical —CN.

The term “cycloalkoxy” as used herein refers to a cycloalkyl group attached to an oxygen (cycloalkyl-O—).

The term “cycloalkyl” as used herein refers to a monocyclic saturated or partially unsaturated hydrocarbon group of for example 3-6, or 4-6 carbons, referred to herein, e.g., as C₃₋₆cycloalkyl or C₄₋₆cycloalkyl and derived from a cycloalkane. Exemplary cycloalkyl groups include, but are not limited to, cyclohexyl, cyclohexenyl, cyclopentyl, cyclobutyl or, cyclopropyl.

The terms “halo” or “halogen” as used herein refer to F, Cl, Br, or I.

The term “heterocyclylalkoxy” as used herein refers to a heterocyclyl-alkyl-O— group.

The term “heterocyclyloxyalkyl” refers to a heterocyclyl-O-alkyl- group.

The term “heterocyclyloxy” refers to a heterocyclyl-O— group.

The term “heteroaryloxy” refers to a heteroaryl-O— group.

The terms “hydroxy” and “hydroxyl” as used herein refers to the radical —OH.

The term “oxo” as used herein refers to the radical ═O.

The term “connector” as used herein to refers to an atom or a collection of atoms optionally used to link interconnecting moieties, such as a disclosed linker and a pharmacophore. Contemplated connectors are generally hydrolytically stable.

“Treating” includes any effect, e.g., lessening, reducing, modulating, or eliminating, that results in the improvement of the condition, disease, disorder and the like.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated is desirably a mammal in which treatment of obesity, or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system, animal, or human that is being sought by the researcher, veterinarian, medical doctor, or other clinician. The compounds are administered in therapeutically effective amounts to treat a disease. Alternatively, a therapeutically effective amount of a compound is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. Various stereoisomers of these compounds and mixtures thereof are encompassed by this disclosure. Stereoisomers include enantiomers and diastereomers. Mixtures of enantiomers or diastereomers may be designated “(±)” in nomenclature, but the skilled artisan will recognize that a structure may denote a chiral center implicitly.

The compounds of the disclosure may contain one or more chiral centers and/or double bonds and, therefore, exist as geometric isomers, enantiomers or diastereomers. The enantiomers and diastereomers may be designated by the symbols “(+),” “(−).” “R” or “S,” depending on the configuration of substituents around the stereogenic carbon atom, but the skilled artisan will recognize that a structure may denote a chiral center implicitly. Geometric isomers, resulting from the arrangement of substituents around a carbon-carbon double bond or arrangement of substituents around a cycloalkyl or heterocyclic ring, can also exist in the compounds. The symbol ═ denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The term “stereoisomers” when used herein consist of all geometric isomers, enantiomers or diastereomers. Various stereoisomers of these compounds and mixtures thereof are encompassed by this disclosure.

Individual enantiomers and diastereomers of the compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like. In one embodiment, the compound is amorphous. In one embodiment, the compound is a polymorph. In another embodiment, the compound is in a crystalline form.

Also embraced are isotopically labeled compounds which are identical to those recited herein, except that one or more atoms are replaced by an atom having an atomic mass or mass number different from the atomic mass or mass number usually found in nature. Examples of isotopes that can be incorporated into the compounds include isotopes of hydrogen, carbon, nitrogen, oxygen, phosphorus, sulfur, fluorine and chlorine, such as ¹⁰B, ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds can generally be prepared by following procedures analogous to those disclosed in the Examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood, or liver). Prodrugs are well known in the art (for example, see Rautio, Kumpulainen, et al, Nature Reviews Drug Discovery 2008, 7, 255). For example, if a compound or a pharmaceutically acceptable salt, hydrate, or solvate of the compound contains a carboxylic acid functional group, a prodrug can comprise an ester formed by the replacement of the hydrogen atom of the acid group with a group such as (C₁₋₈)alkyl, (C₂₋₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound contains an alcohol functional group, a prodrug can be formed by the replacement of the hydrogen atom of the alcohol group with a group such as (C₁₋6)alkanoyloxymethyl, 1-((C₁₋₆)alkanoyloxy)ethyl, 1-methyl-1-((C₁₋₆)alkanoyloxy)ethyl (C₁₋₆)alkoxycarbonyloxymethyl, N—(C₁₋₆)alkoxycarbonylaminomethyl, succinoyl, (C₁₋₆)alkanoyl, α-amino(C₁₋₄ alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound incorporates an amine functional group, a prodrug can be formed, for example, by creation of an amide or carbamate, an N-acyloxyakyl derivative, an (oxodioxolenyl)methyl derivative, an N-Mannich base, imine, or enamine. In addition, a secondary amine can be metabolically cleaved to generate a bioactive primary amine, or a tertiary amine can be metabolically cleaved to generate a bioactive primary or secondary amine. For examples, see Simplicio, et al., Molecules 2008, 13, 519 and references therein.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EXAMPLES

The compounds described herein can be prepared in a number of ways based on the teachings contained herein and synthetic procedures known in the art.

### Examples 1-20

The following table (Table R) contains boronic “hetero” monomers which may form a dimer with the monomers (e.g. diol monomers) of examples 21-43.

TABLE R

| | Compound Structure |
|---|---|
| No. | (e.g., X—Y—Z) |
| 1 | 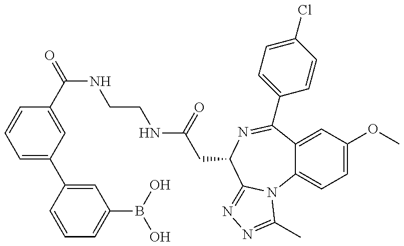 [SMILES: COc1ccc2c(c1)C(c1ccc(Cl)cc1)=N[C@@H](CC(=O)NCCNC(=O)c1cccc(-c3cccc(B(O)O)c3)c1)c1nnc(C)n1-2] |
| 2 | 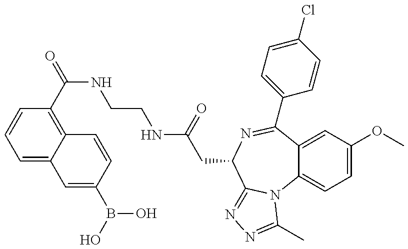 [SMILES: COc1ccc2c(c1)C(c1ccc(Cl)cc1)=N[C@@H](CC(=O)NCCNC(=O)c1cccc3cc(B(O)O)ccc13)c1nnc(C)n1-2] |
| 3 | 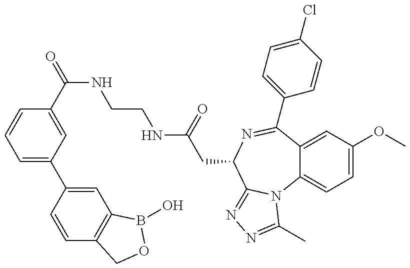 [SMILES: COc1ccc2c(c1)C(c1ccc(Cl)cc1)=N[C@@H](CC(=O)NCCNC(=O)c1cccc(-c3ccc4c(c3)B(O)OC4)c1)c1nnc(C)n1-2] |
| 4 | 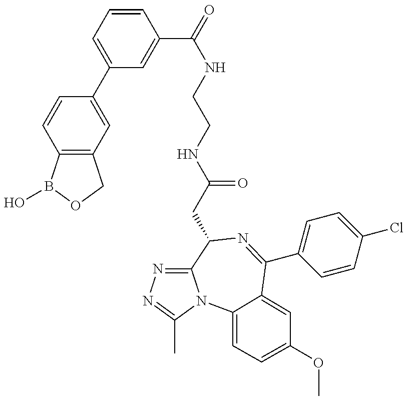 [SMILES: COc1ccc2c(c1)C(c1ccc(Cl)cc1)=N[C@@H](CC(=O)NCCNC(=O)c1cccc(-c3ccc4c(c3)COB4O)c1)c1nnc(C)n1-2] |
| 5 | 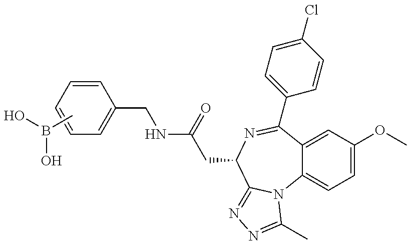 |
| 6 | 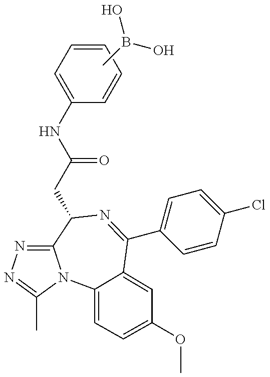 |
| 7 | 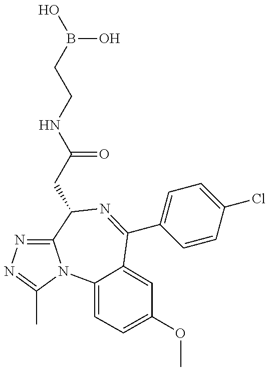 [SMILES: COc1ccc2c(c1)C(c1ccc(Cl)cc1)=N[C@@H](CC(=O)NCCB(O)O)c1nnc(C)n1-2] |
| 8 | 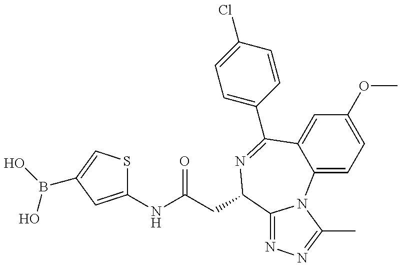 [SMILES: COc1ccc2c(c1)C(c1ccc(Cl)cc1)=N[C@@H](CC(=O)Nc1cc(B(O)O)cs1)c1nnc(C)n1-2] |
| 9 | 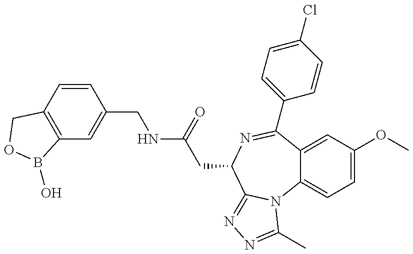 [SMILES: COc1ccc2c(c1)C(c1ccc(Cl)cc1)=N[C@@H](CC(=O)NCc1ccc3c(c1)B(O)OC3)c1nnc(C)n1-2] |
| 10 | 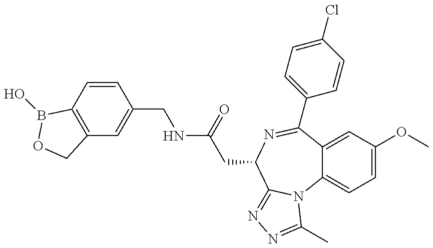 [SMILES: COc1ccc2c(c1)C(c1ccc(Cl)cc1)=N[C@@H](CC(=O)NCc1ccc3c(c1)COB3O)c1nnc(C)n1-2] |
| 11 | 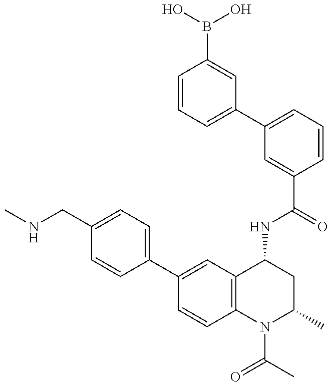 [SMILES: CNCc1ccc(-c2ccc3c(c2)[C@H](NC(=O)c2cccc(-c4cccc(B(O)O)c4)c2)C[C@H](C)N3C(C)=O)cc1] |
| 12 | 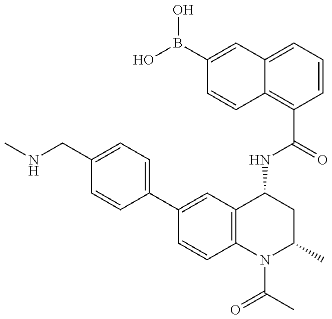 [SMILES: CNCc1ccc(-c2ccc3c(c2)[C@H](NC(=O)c2cccc4cc(B(O)O)ccc24)C[C@H](C)N3C(C)=O)cc1] |
| 13 | 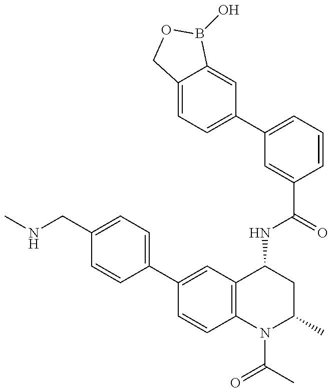 [SMILES: CNCc1ccc(-c2ccc3c(c2)[C@H](NC(=O)c2cccc(-c4ccc5c(c4)B(O)OC5)c2)C[C@H](C)N3C(C)=O)cc1] |
| 14 | 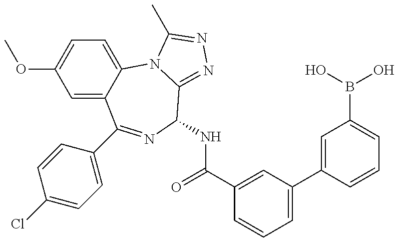 [SMILES: COc1ccc2c(c1)C(c1ccc(Cl)cc1)=N[C@@H](NC(=O)c1cccc(-c3cccc(B(O)O)c3)c1)c1nnc(C)n1-2] |
| 15 | 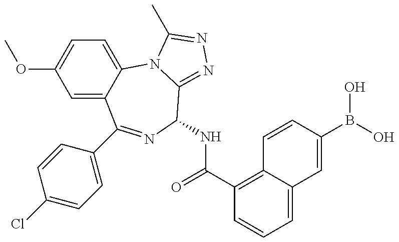 [SMILES: COc1ccc2c(c1)C(c1ccc(Cl)cc1)=N[C@@H](NC(=O)c1cccc3cc(B(O)O)ccc13)c1nnc(C)n1-2] |
| 16 | 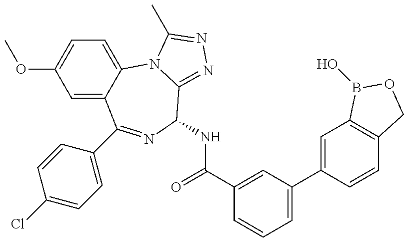 [SMILES: COc1ccc2c(c1)C(c1ccc(Cl)cc1)=N[C@@H](NC(=O)c1cccc(-c3ccc4c(c3)B(O)OC4)c1)c1nnc(C)n1-2] |
| 17 | 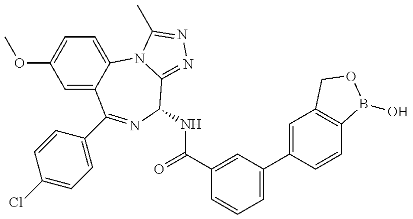 [SMILES: COc1ccc2c(c1)C(c1ccc(Cl)cc1)=N[C@@H](NC(=O)c1cccc(-c3ccc4c(c3)COB4O)c1)c1nnc(C)n1-2] |
| 18 | 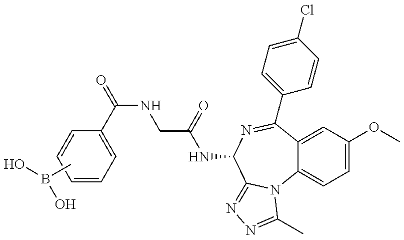 |
| 19 | 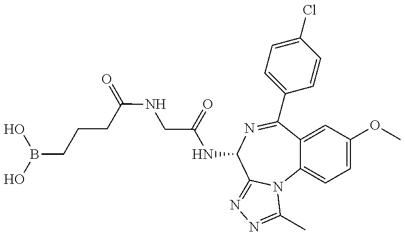 [SMILES: COc1ccc2c(c1)C(c1ccc(Cl)cc1)=N[C@@H](NC(=O)CNC(=O)CCCB(O)O)c1nnc(C)n1-2] |
| 20 | 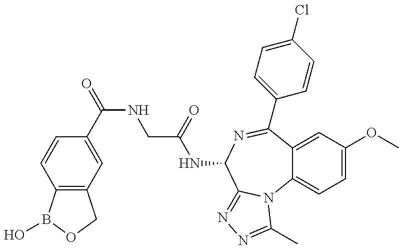 [SMILES: COc1ccc2c(c1)C(c1ccc(Cl)cc1)=N[C@@H](NC(=O)CNC(=O)c1ccc3c(c1)COB3O)c1nnc(C)n1-2] |

### Examples 21-43

The following table (Table S) contains “hetero” monomers which may form a dimer with the monomers (e.g., boronic acid or boronate monomers) of examples 1-20, e.g. Table S contains “diol” monomers.

TABLE S

| | Compound Structure |
|---|---|
| No. | (e.g., X—Y—Z) |
| 21 | 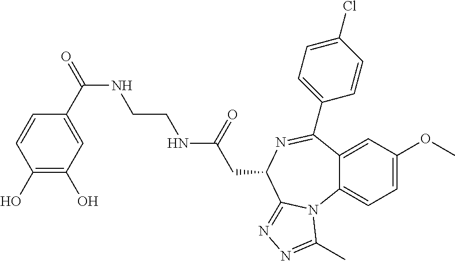 [SMILES: COc1ccc2c(c1)C(c1ccc(Cl)cc1)=N[C@@H](CC(=O)NCCNC(=O)c1ccc(O)c(O)c1)c1nnc(C)n1-2] |
| 22 | 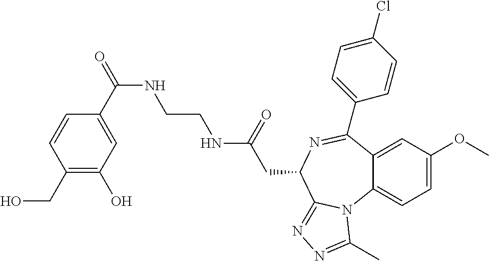 [SMILES: COc1ccc2c(c1)C(c1ccc(Cl)cc1)=N[C@@H](CC(=O)NCCNC(=O)c1ccc(CO)c(O)c1)c1nnc(C)n1-2] |
| 23 | 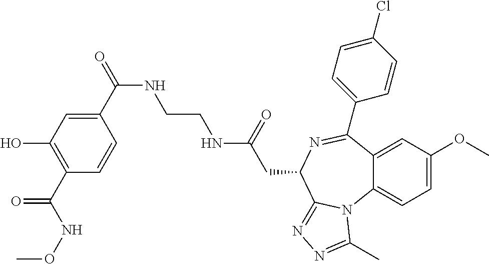 [SMILES: CONC(=O)c1ccc(C(=O)NCCNC(=O)C[C@@H]2N=C(c3ccc(Cl)cc3)c3cc(OC)ccc3-n3c(C)nnc32)cc1O] |
| 24 | 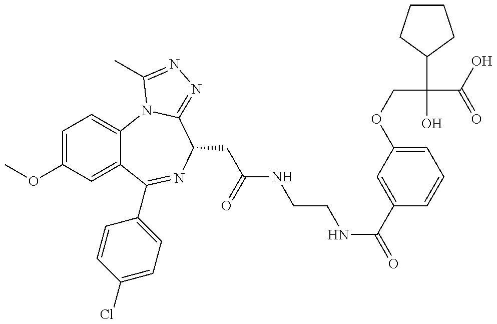 [SMILES: COc1ccc2c(c1)C(c1ccc(Cl)cc1)=N[C@@H](CC(=O)NCCNC(=O)c1cccc(OCC(O)(C(=O)O)C3CCCC3)c1)c1nnc(C)n1-2] |
| 25 | 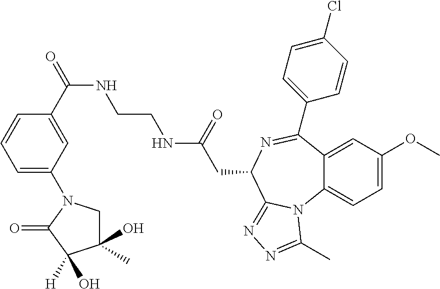 [SMILES: COc1ccc2c(c1)C(c1ccc(Cl)cc1)=N[C@@H](CC(=O)NCCNC(=O)c1cccc(N3C[C@@](C)(O)[C@@H](O)C3=O)c1)c1nnc(C)n1-2] |
| 26 | 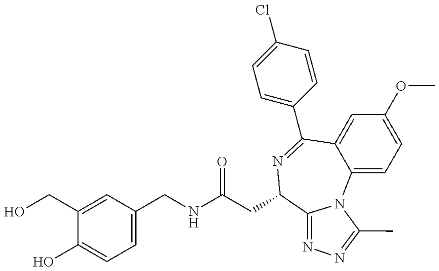 [SMILES: COc1ccc2c(c1)C(c1ccc(Cl)cc1)=N[C@@H](CC(=O)NCc1ccc(O)c(CO)c1)c1nnc(C)n1-2] |
| 27 | 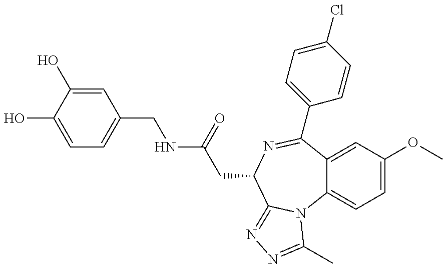 [SMILES: COc1ccc2c(c1)C(c1ccc(Cl)cc1)=N[C@@H](CC(=O)NCc1ccc(O)c(O)c1)c1nnc(C)n1-2] |
| 28 | 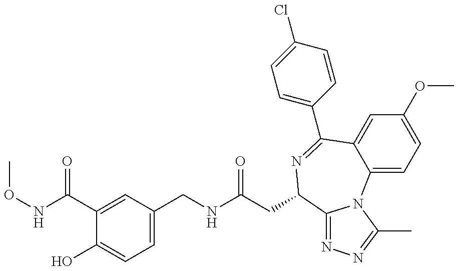 [SMILES: CONC(=O)c1cc(CNC(=O)C[C@@H]2N=C(c3ccc(Cl)cc3)c3cc(OC)ccc3-n3c(C)nnc32)ccc1O] |
| 29 | 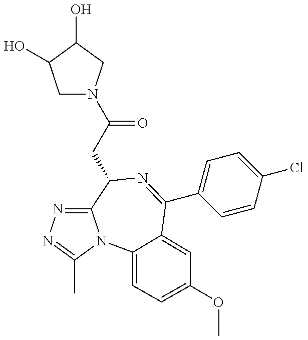 [SMILES: COc1ccc2c(c1)C(c1ccc(Cl)cc1)=N[C@@H](CC(=O)N1CC(O)C(O)C1)c1nnc(C)n1-2] |
| 30 | 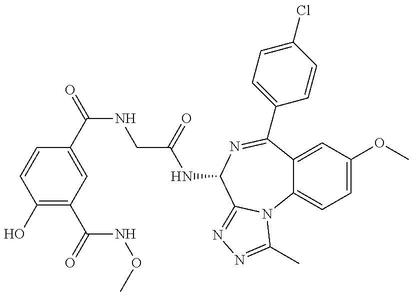 [SMILES: CONC(=O)c1cc(C(=O)NCC(=O)N[C@@H]2N=C(c3ccc(Cl)cc3)c3cc(OC)ccc3-n3c(C)nnc32)ccc1O] |
| 31 | 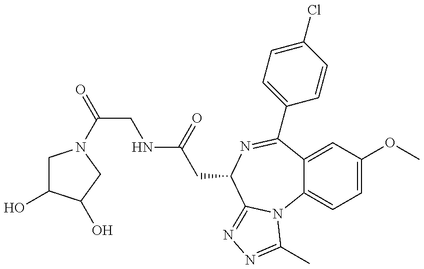 [SMILES: COc1ccc2c(c1)C(c1ccc(Cl)cc1)=N[C@@H](CC(=O)NCC(=O)N1CC(O)C(O)C1)c1nnc(C)n1-2] |
| 32 | 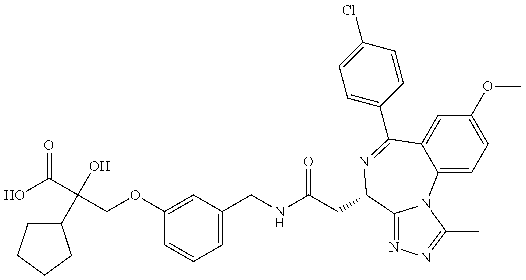 [SMILES: COc1ccc2c(c1)C(c1ccc(Cl)cc1)=N[C@@H](CC(=O)NCc1cccc(OCC(O)(C(=O)O)C3CCCC3)c1)c1nnc(C)n1-2] |
| 33 | 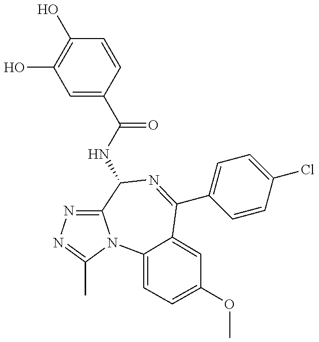 [SMILES: COc1ccc2c(c1)C(c1ccc(Cl)cc1)=N[C@@H](NC(=O)c1ccc(O)c(O)c1)c1nnc(C)n1-2] |
| 34 | 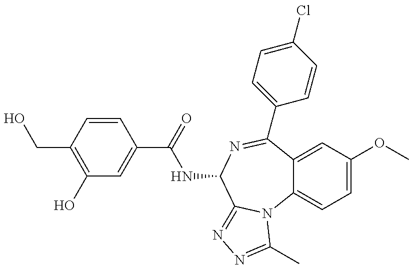 [SMILES: COc1ccc2c(c1)C(c1ccc(Cl)cc1)=N[C@@H](NC(=O)c1ccc(CO)c(O)c1)c1nnc(C)n1-2] |
| 35 | 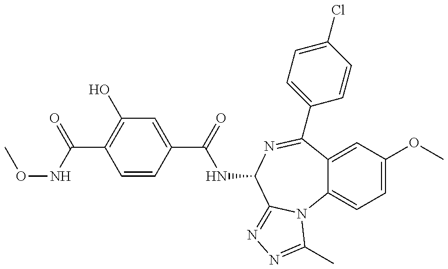 |
| 36 | 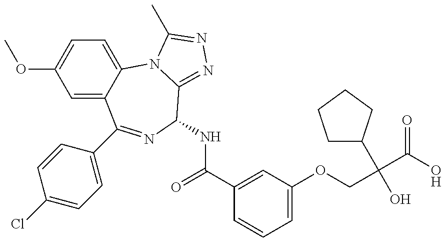 [SMILES: COc1ccc2c(c1)C(c1ccc(Cl)cc1)=N[C@@H](NC(=O)c1cccc(OCC(O)(C(=O)O)C3CCCC3)c1)c1nnc(C)n1-2] |
| 37 | 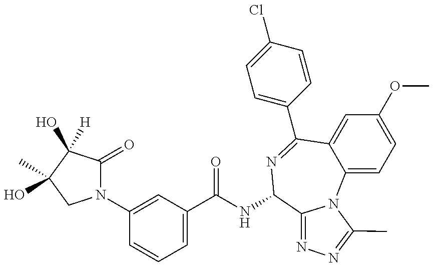 [SMILES: COc1ccc2c(c1)C(c1ccc(Cl)cc1)=N[C@@H](NC(=O)c1cccc(N3C[C@@](C)(O)[C@@H](O)C3=O)c1)c1nnc(C)n1-2] |
| 38 | 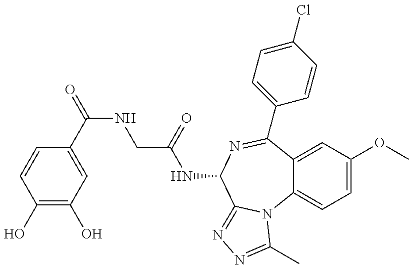 [SMILES: COc1ccc2c(c1)C(c1ccc(Cl)cc1)=N[C@@H](NC(=O)CNC(=O)c1ccc(O)c(O)c1)c1nnc(C)n1-2] |
| 39 | 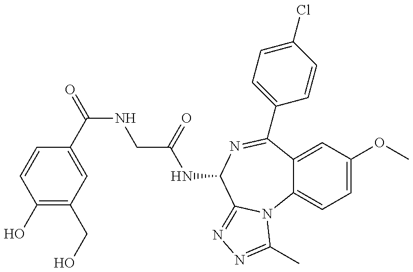 [SMILES: COc1ccc2c(c1)C(c1ccc(Cl)cc1)=N[C@@H](NC(=O)CNC(=O)c1ccc(O)c(CO)c1)c1nnc(C)n1-2] |
| 40 | 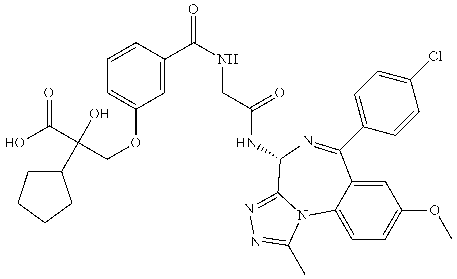 [SMILES: COc1ccc2c(c1)C(c1ccc(Cl)cc1)=N[C@@H](NC(=O)CNC(=O)c1cccc(OCC(O)(C(=O)O)C3CCCC3)c1)c1nnc(C)n1-2] |
| 41 | 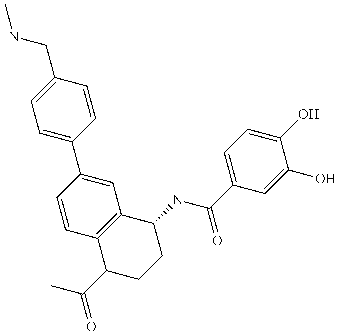 [SMILES: CNCc1ccc(-c2ccc3c(c2)[C@H](NC(=O)c2ccc(O)c(O)c2)CCC3C(C)=O)cc1] |
| 42 | 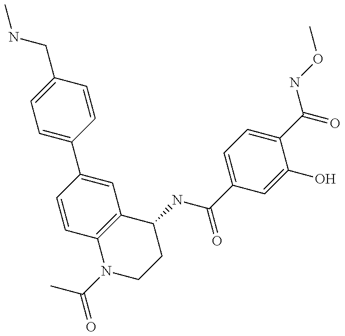 [SMILES: CNCc1ccc(-c2ccc3c(c2)[C@H](NC(=O)c2ccc(C(=O)NOC)c(O)c2)CCN3C(C)=O)cc1] |
| 43 | 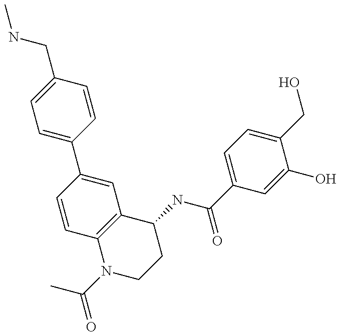 [SMILES: CNCc1ccc(-c2ccc3c(c2)[C@H](NC(=O)c2ccc(CO)c(O)c2)CCN3C(C)=O)cc1] |

### Examples 44-52

The following table (Table T) contains “homo” monomers which may form a dimer with another homo monomer that may be the same or different.

TABLE T

| | Compound Structure |
|---|---|
| No. | (e.g., X—Y—Z) |
| 44 | 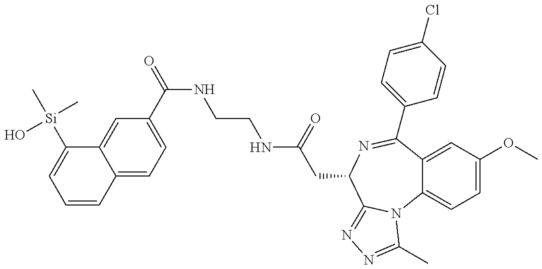 [SMILES: COc1ccc2c(c1)C(c1ccc(Cl)cc1)=N[C@@H](CC(=O)NCCNC(=O)c1ccc3cccc([Si](C)(C)O)c3c1)c1nnc(C)n1-2] |
| 45 | 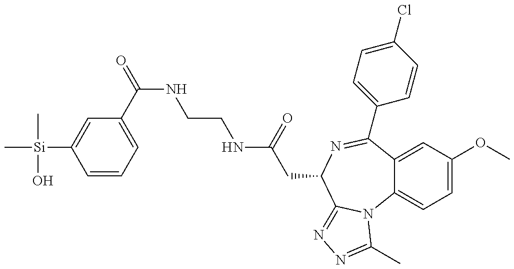 |
| 46 | 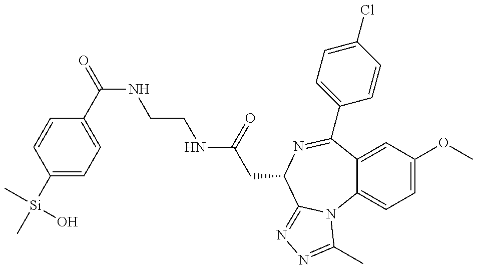 |
| 47 | 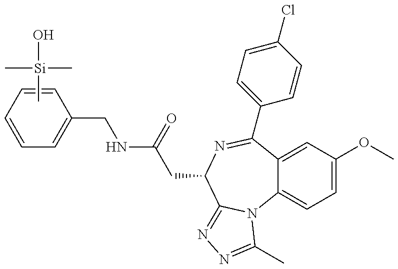 |
| 48 | 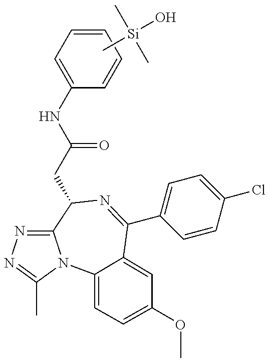 |
| 49 | 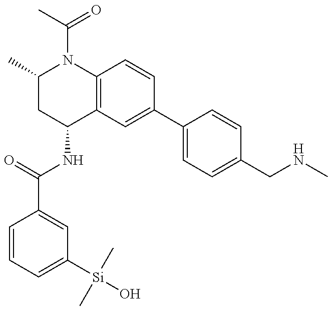 [SMILES: CNCc1ccc(-c2ccc3c(c2)[C@H](NC(=O)c2cccc([Si](C)(C)O)c2)C[C@H](C)N3C(C)=O)cc1] |
| 50 | 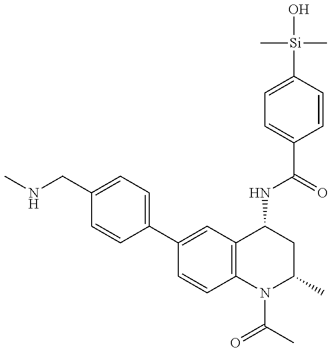 |
| 51 | 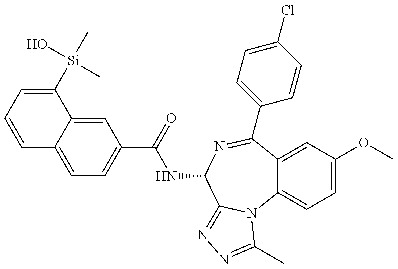 [SMILES: COc1ccc2c(c1)C(c1ccc(Cl)cc1)=N[C@@H](NC(=O)c1ccc3cccc([Si](C)(C)O)c3c1)c1nnc(C)n1-2] |
| 52 | 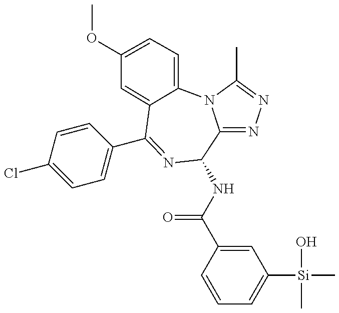 [SMILES: COc1ccc2c(c1)C(c1ccc(Cl)cc1)=N[C@@H](NC(=O)c1cccc([Si](C)(C)O)c1)c1nnc(C)n1-2] |

### Example 53

### Preparation of 2-[(4S)-6-(4-Chlorophenyl)-1-methyl-8-hydroxy-4H-[1,2,4]triazolo[4,3-a][1,4]benzodiazepine-4-yl]-N-ethylacetamide (Compound 9)

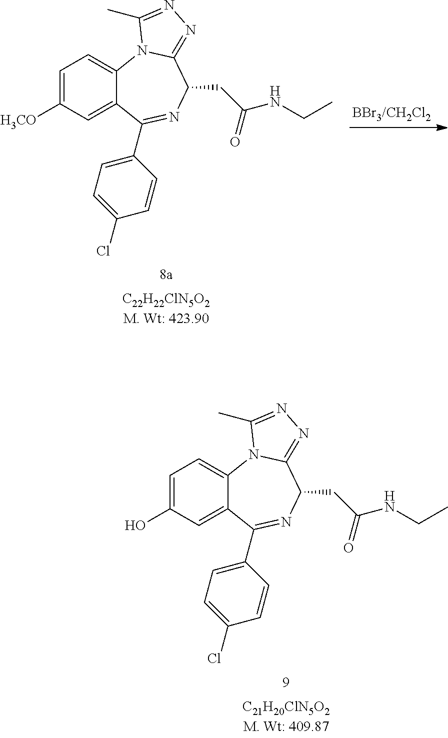 [SMILES: CCNC(=O)C[C@@H]1N=C(c2ccc(Cl)cc2)c2cc(OC)ccc2-n2c(C)nnc21 ; CCNC(=O)C[C@@H]1N=C(c2ccc(Cl)cc2)c2cc(O)ccc2-n2c(C)nnc21]

A solution of methoxy compound 8a (12.4 g, 29.3 mmole) in methylene chloride (300 mL) was cooled to −15° C. using ice salt bath and then added BBr₃ (14 mL, 145 mmole). The temperature was allowed to rise to room temperature and continued stirring overnight. At this point the TLC (5% MeOH/CH₂Cl₂) showed complete disappearance of starting material. The reaction mixture was quenched into a mixture of ice-cold saturated aqueous NaHCO₃ (600 mL) containing 10% methanol in methylene chloride (200 mL). It was stirred for 2 h and the organic layer was separated. The aqueous layer was extracted one more time with 10% methanol in methylene chloride (100 mL) and the combined organic layers were washed with saturated aqueous NaHCO₃ (2 x 100 mL), dried over Na₂SO₄, filtered and concentrated. The crude mixture was purified by silica gel column chromatography using 4-6% methanol in methylene chloride. All the fractions containing required compound were collected, concentrated and the residue was triturated with hot hexane. It was cooled to room temperature, filtered, washed with hexane and dried in vacuum oven at 50-55° C. over P₂O₅ to give pure compound 9 (9.7 g, 82%). Mp180-182° C. ¹H NMR (DMSO-d₆) δ 10.21 (br s, 1H), 8.19 (t, J=5.4 Hz, 1H), 7.62 (d, J=8.8 Hz, 1H), 7.46 (m, 4H), 7.13 (dd, J=2.8 & 8.8 Hz, 1H), 6.69 (d, J=2.8 Hz, 1H), 4.45 (q, J=5.6 & 2.8 Hz, 1H), 3.12 (m, 4H), 2.50 (s, 3H), 1.03 (t, J=7.2 Hz, 3H); ¹³C NMR (DMSO-d₆) δ 169.37, 165.81, 155.96, 155.77, 150.60, 137.52, 135.33, 131.04, 129.38, 128.25, 125.55, 124.98, 119.08, 116.34, 53.31, 37.63, 33.43, 14.82, 11.49; MS (ESI) m/z 410 (M+H)+. [α]_{d}+76.9 (c=1 in MeOH).

### Examples 54-65

The following table (Table U″) contains monomers which may form a dimer with a second monomer. The monomer examples in Table U″, having boronic acid linkers (Z), correspond to the structures seen in Table A″, above. As described above a first monomer that has a boronic acid linker may be capable of forming a multimer with a second monomer that has a diol linker.

TABLE U″

| No. | Compound Structure (X—Y—Z) |
|---|---|
| 54 | 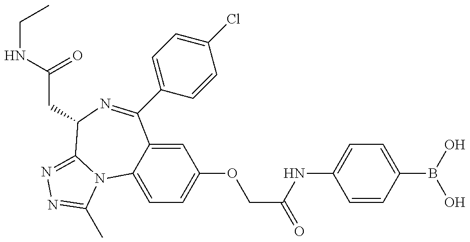 [SMILES: CCNC(=O)C[C@@H]1N=C(c2ccc(Cl)cc2)c2cc(OCC(=O)Nc3ccc(B(O)O)cc3)ccc2-n2c(C)nnc21] |
| 55 | 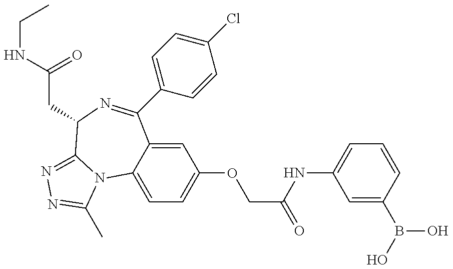 [SMILES: CCNC(=O)C[C@@H]1N=C(c2ccc(Cl)cc2)c2cc(OCC(=O)Nc3cccc(B(O)O)c3)ccc2-n2c(C)nnc21] |
| 56 | 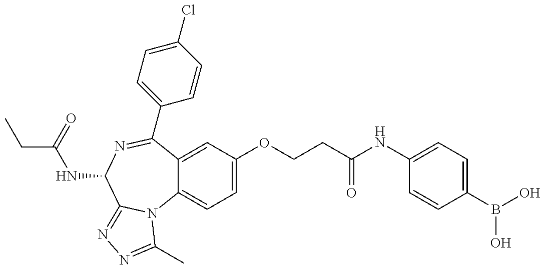 [SMILES: CCC(=O)N[C@@H]1N=C(c2ccc(Cl)cc2)c2cc(OCCC(=O)Nc3ccc(B(O)O)cc3)ccc2-n2c(C)nnc21] |
| 57 | 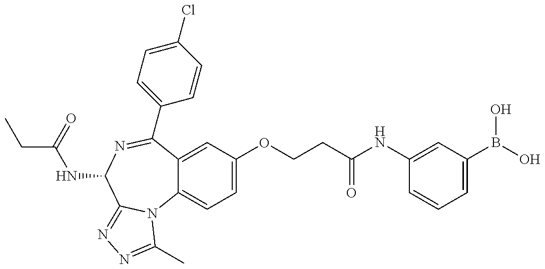 [SMILES: CCC(=O)N[C@@H]1N=C(c2ccc(Cl)cc2)c2cc(OCCC(=O)Nc3cccc(B(O)O)c3)ccc2-n2c(C)nnc21] |
| 58 | 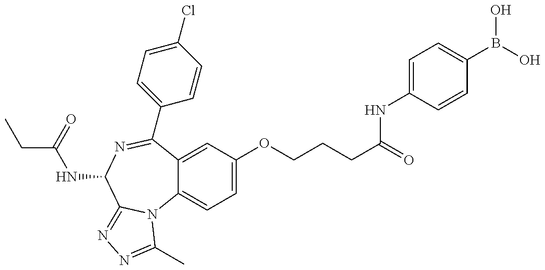 [SMILES: CCC(=O)N[C@@H]1N=C(c2ccc(Cl)cc2)c2cc(OCCCC(=O)Nc3ccc(B(O)O)cc3)ccc2-n2c(C)nnc21] |
| 59 | 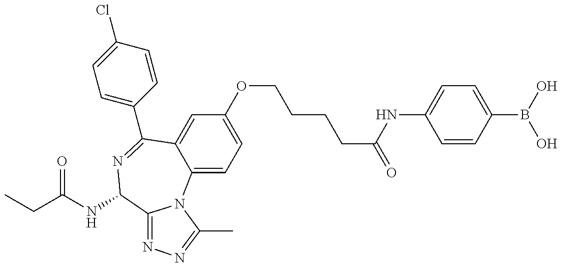 [SMILES: CCC(=O)N[C@@H]1N=C(c2ccc(Cl)cc2)c2cc(OCCCCC(=O)Nc3ccc(B(O)O)cc3)ccc2-n2c(C)nnc21] |
| 60 | 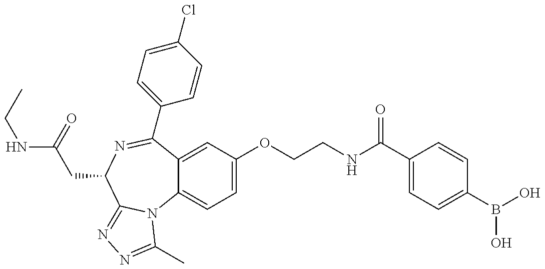 [SMILES: CCNC(=O)C[C@@H]1N=C(c2ccc(Cl)cc2)c2cc(OCCNC(=O)c3ccc(B(O)O)cc3)ccc2-n2c(C)nnc21] |
| 61 | 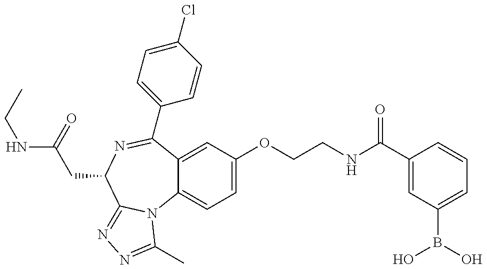 [SMILES: CCNC(=O)C[C@@H]1N=C(c2ccc(Cl)cc2)c2cc(OCCNC(=O)c3cccc(B(O)O)c3)ccc2-n2c(C)nnc21] |
| 62 | 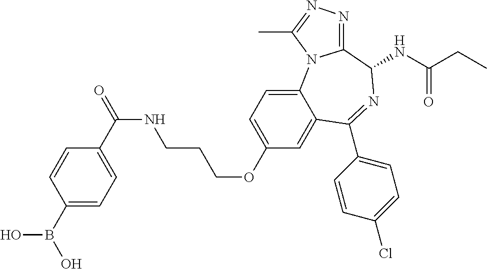 [SMILES: CCC(=O)N[C@@H]1N=C(c2ccc(Cl)cc2)c2cc(OCCCNC(=O)c3ccc(B(O)O)cc3)ccc2-n2c(C)nnc21] |
| 63 | 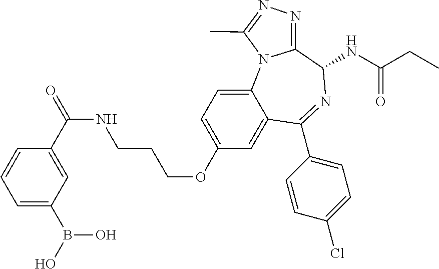 [SMILES: CCC(=O)N[C@@H]1N=C(c2ccc(Cl)cc2)c2cc(OCCCNC(=O)c3cccc(B(O)O)c3)ccc2-n2c(C)nnc21] |
| 64 | 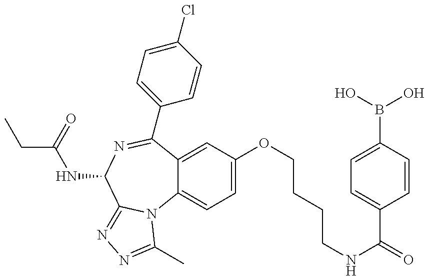 [SMILES: CCC(=O)N[C@@H]1N=C(c2ccc(Cl)cc2)c2cc(OCCCCNC(=O)c3ccc(B(O)O)cc3)ccc2-n2c(C)nnc21] |
| 65 | 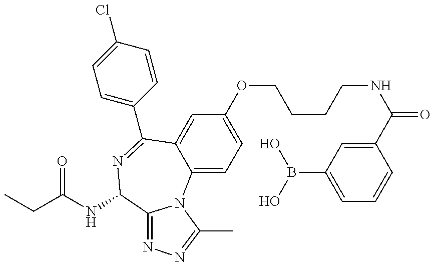 [SMILES: CCC(=O)N[C@@H]1N=C(c2ccc(Cl)cc2)c2cc(OCCCCNC(=O)c3cccc(B(O)O)c3)ccc2-n2c(C)nnc21] |

### Examples 66-89

Table V″ contains monomers which may form a dimer with a second monomer. The monomer examples in Table V″, having boronic acid, diol or silanol linkers (Z), correspond to the structures in Table B″. A first monomer that has a boronic acid linker may be capable of forming a multimer with a second monomer that has a diol linker. Afirst monomer that has a silanol linker may be capable of forming a multimer with a second monomer that has the same or different silanol linker.

TABLE V″

| | Compound Structure |
|---|---|
| No. | (e.g., X—Y—Z) |
| 66 | 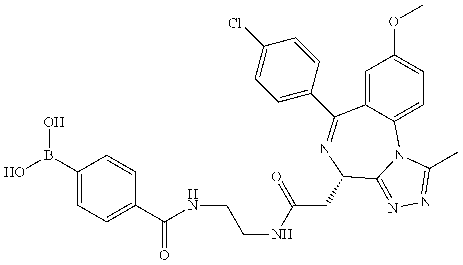 [SMILES: COc1ccc2c(c1)C(c1ccc(Cl)cc1)=N[C@@H](CC(=O)NCCNC(=O)c1ccc(B(O)O)cc1)c1nnc(C)n1-2] |
| 67 | 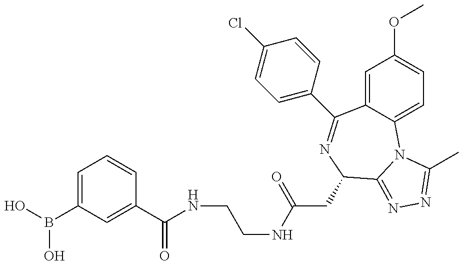 [SMILES: COc1ccc2c(c1)C(c1ccc(Cl)cc1)=N[C@@H](CC(=O)NCCNC(=O)c1cccc(B(O)O)c1)c1nnc(C)n1-2] |
| 68 | 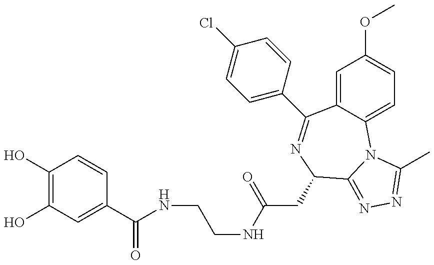 [SMILES: COc1ccc2c(c1)C(c1ccc(Cl)cc1)=N[C@@H](CC(=O)NCCNC(=O)c1ccc(O)c(O)c1)c1nnc(C)n1-2] |
| 69 | 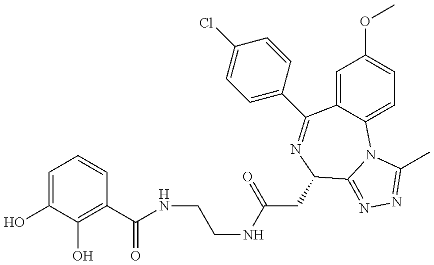 [SMILES: COc1ccc2c(c1)C(c1ccc(Cl)cc1)=N[C@@H](CC(=O)NCCNC(=O)c1cccc(O)c1O)c1nnc(C)n1-2] |
| 70 | 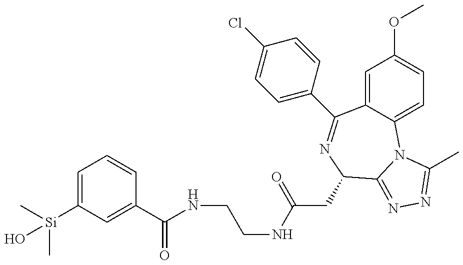 |
| 71 | 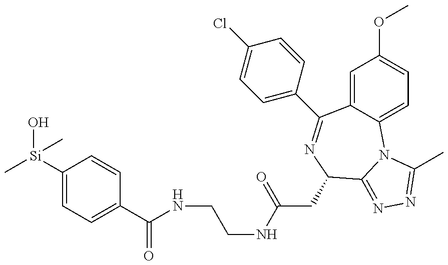 |
| 72 | 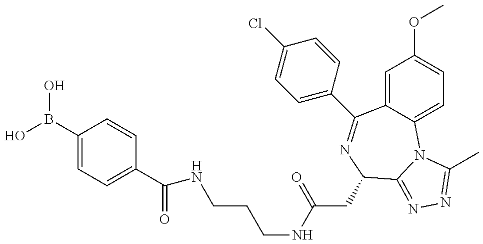 [SMILES: COc1ccc2c(c1)C(c1ccc(Cl)cc1)=N[C@@H](CC(=O)NCCCNC(=O)c1ccc(B(O)O)cc1)c1nnc(C)n1-2] |
| 73 | 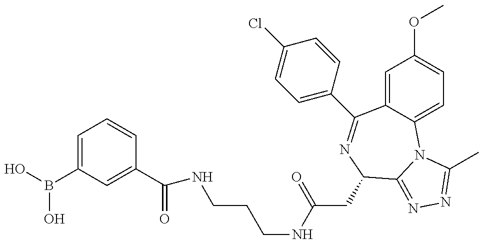 [SMILES: COc1ccc2c(c1)C(c1ccc(Cl)cc1)=N[C@@H](CC(=O)NCCCNC(=O)c1cccc(B(O)O)c1)c1nnc(C)n1-2] |
| 74 | 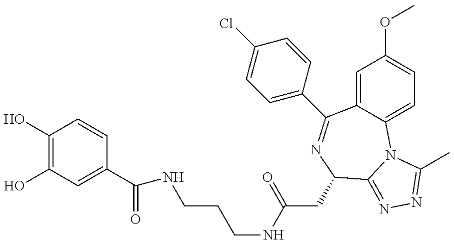 [SMILES: COc1ccc2c(c1)C(c1ccc(Cl)cc1)=N[C@@H](CC(=O)NCCCNC(=O)c1ccc(O)c(O)c1)c1nnc(C)n1-2] |
| 75 | 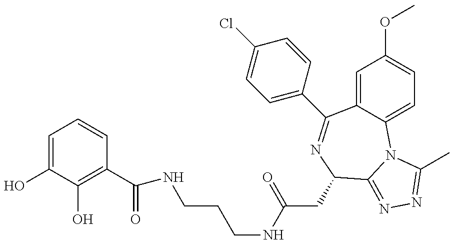 [SMILES: COc1ccc2c(c1)C(c1ccc(Cl)cc1)=N[C@@H](CC(=O)NCCCNC(=O)c1cccc(O)c1O)c1nnc(C)n1-2] |
| 76 | 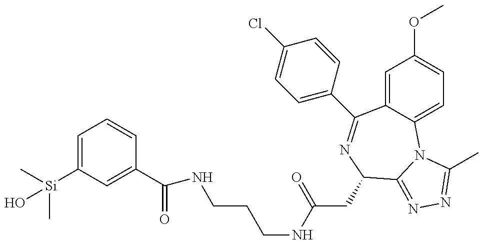 |
| 77 | 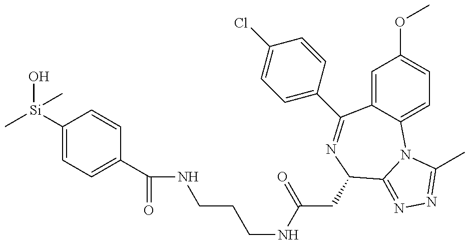 |
| 78 | 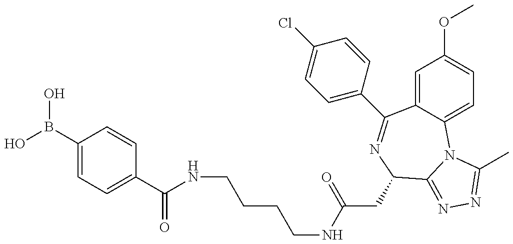 [SMILES: COc1ccc2c(c1)C(c1ccc(Cl)cc1)=N[C@@H](CC(=O)NCCCCNC(=O)c1ccc(B(O)O)cc1)c1nnc(C)n1-2] |
| 79 | 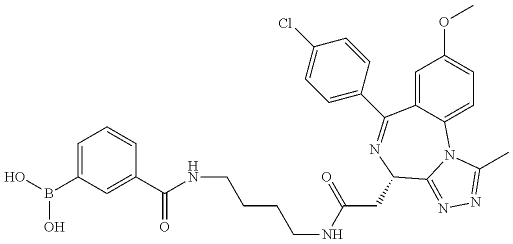 [SMILES: COc1ccc2c(c1)C(c1ccc(Cl)cc1)=N[C@@H](CC(=O)NCCCCNC(=O)c1cccc(B(O)O)c1)c1nnc(C)n1-2] |
| 80 | 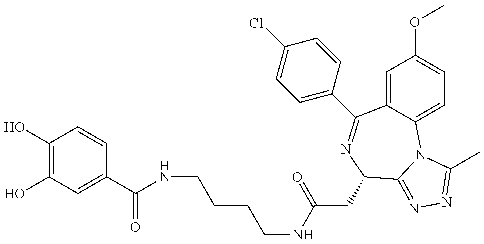 [SMILES: COc1ccc2c(c1)C(c1ccc(Cl)cc1)=N[C@@H](CC(=O)NCCCCNC(=O)c1ccc(O)c(O)c1)c1nnc(C)n1-2] |
| 81 | 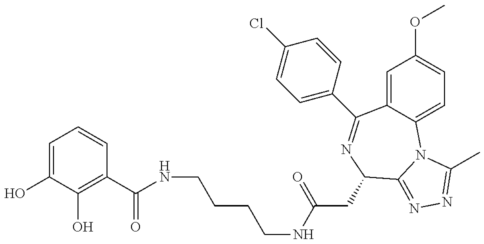 [SMILES: COc1ccc2c(c1)C(c1ccc(Cl)cc1)=N[C@@H](CC(=O)NCCCCNC(=O)c1cccc(O)c1O)c1nnc(C)n1-2] |
| 82 | 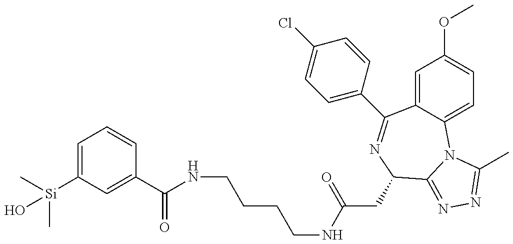 |
| 83 | 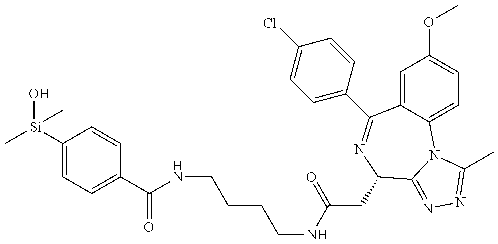 |
| 84 | 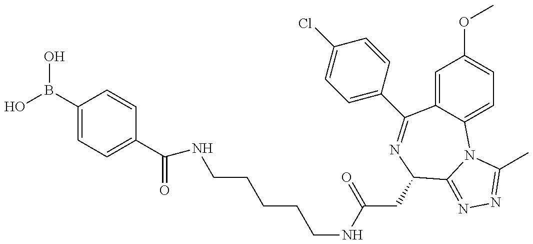 [SMILES: COc1ccc2c(c1)C(c1ccc(Cl)cc1)=N[C@@H](CC(=O)NCCCCCNC(=O)c1ccc(B(O)O)cc1)c1nnc(C)n1-2] |
| 85 | 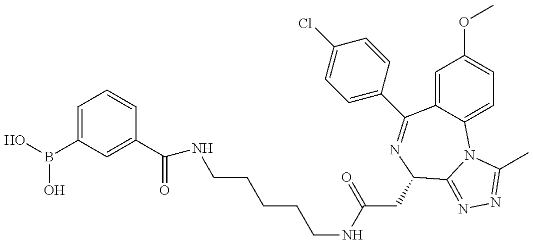 [SMILES: COc1ccc2c(c1)C(c1ccc(Cl)cc1)=N[C@@H](CC(=O)NCCCCCNC(=O)c1cccc(B(O)O)c1)c1nnc(C)n1-2] |
| 86 | 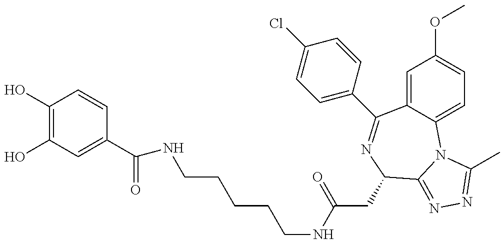 [SMILES: COc1ccc2c(c1)C(c1ccc(Cl)cc1)=N[C@@H](CC(=O)NCCCCCNC(=O)c1ccc(O)c(O)c1)c1nnc(C)n1-2] |
| 87 | 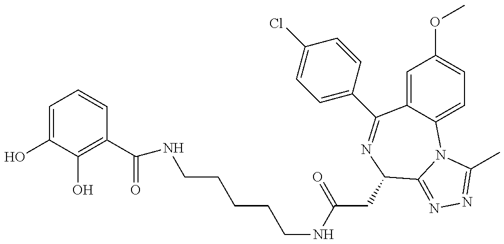 [SMILES: COc1ccc2c(c1)C(c1ccc(Cl)cc1)=N[C@@H](CC(=O)NCCCCCNC(=O)c1cccc(O)c1O)c1nnc(C)n1-2] |
| 88 | 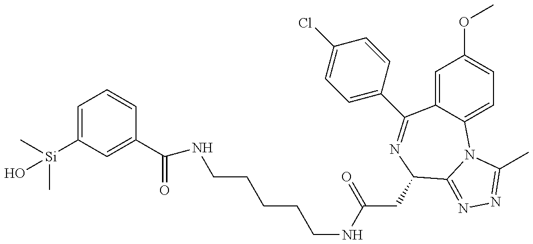 |
| 89 | 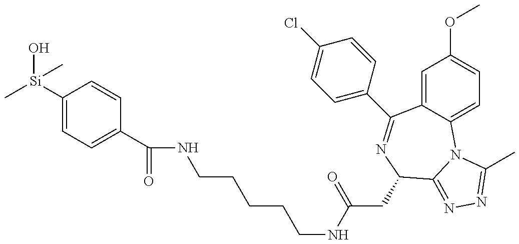 |

### Examples 90-144

Table W contains monomers which may form a dimer with a second monomer. The monomer examples in Table W, having boronic acid, diol or silanol linkers (Z), correspond to the structures in Table C.

TABLE W

| No. | Compound Structure (e.g., X—Y—Z) |
|---|---|
| 91 | 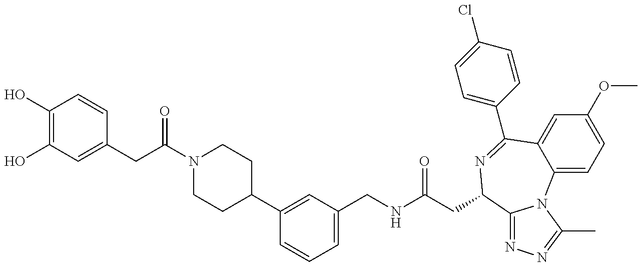 [SMILES: COc1ccc2c(c1)C(c1ccc(Cl)cc1)=N[C@@H](CC(=O)NCc1cccc(C3CCN(C(=O)Cc4ccc(O)c(O)c4)CC3)c1)c1nnc(C)n1-2] |
| 92 | 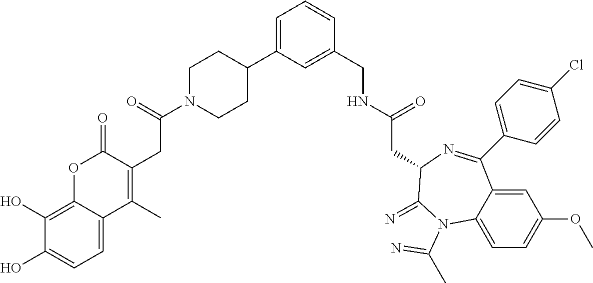 [SMILES: COc1ccc2c(c1)C(c1ccc(Cl)cc1)=N[C@@H](CC(=O)NCc1cccc(C3CCN(C(=O)Cc4c(C)c5ccc(O)c(O)c5oc4=O)CC3)c1)C(=N)N2C(C)=N] |
| 93 | 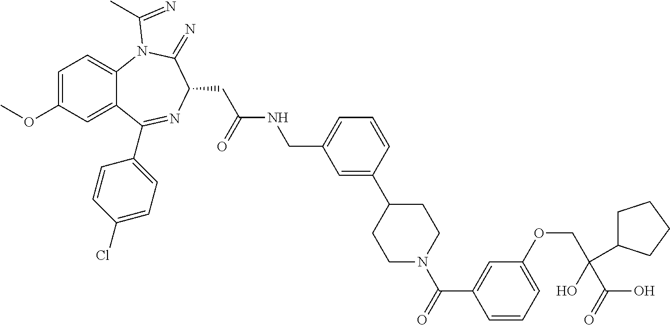 [SMILES: COc1ccc2c(c1)C(c1ccc(Cl)cc1)=N[C@@H](CC(=O)NCc1cccc(C3CCN(C(=O)c4cccc(OCC(O)(C(=O)O)C5CCCC5)c4)CC3)c1)C(=N)N2C(C)=N] |
| 94 | 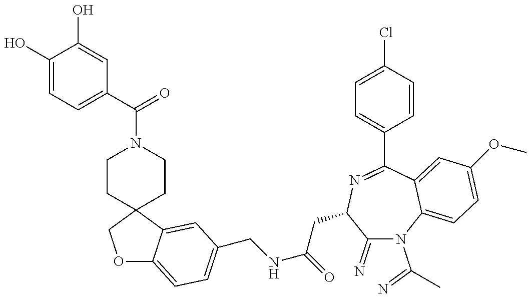 [SMILES: COc1ccc2c(c1)C(c1ccc(Cl)cc1)=N[C@@H](CC(=O)NCc1ccc3c(c1)C1(CCN(C(=O)c4ccc(O)c(O)c4)CC1)CO3)C(=N)N2C(C)=N] |
| 95 | 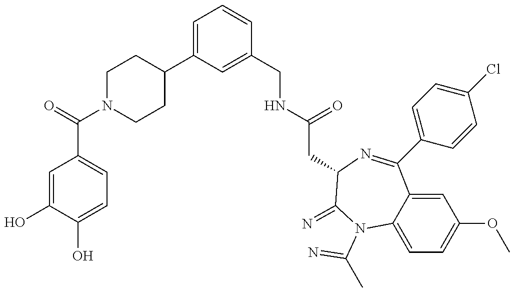 [SMILES: COc1ccc2c(c1)C(c1ccc(Cl)cc1)=N[C@@H](CC(=O)NCc1cccc(C3CCN(C(=O)c4ccc(O)c(O)c4)CC3)c1)C(=N)N2C(C)=N] |
| 96 | 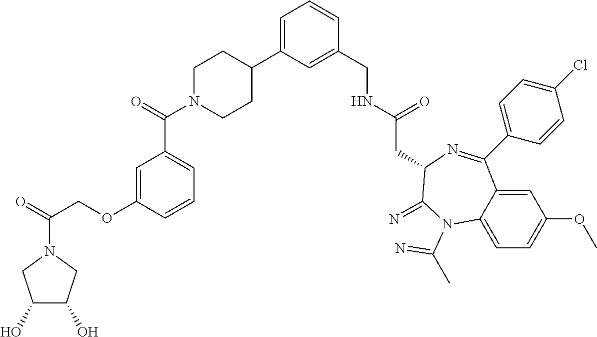 [SMILES: COc1ccc2c(c1)C(c1ccc(Cl)cc1)=N[C@@H](CC(=O)NCc1cccc(C3CCN(C(=O)c4cccc(OCC(=O)N5C[C@@H](O)[C@@H](O)C5)c4)CC3)c1)C(=N)N2C(C)=N] |
| 97 | 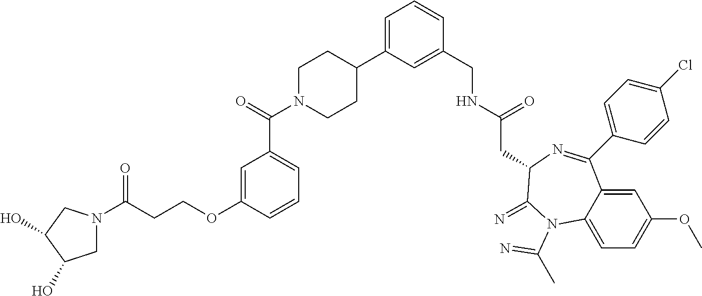 [SMILES: COc1ccc2c(c1)C(c1ccc(Cl)cc1)=N[C@@H](CC(=O)NCc1cccc(C3CCN(C(=O)c4cccc(OCCC(=O)N5C[C@@H](O)[C@@H](O)C5)c4)CC3)c1)C(=N)N2C(C)=N] |
| 98 | 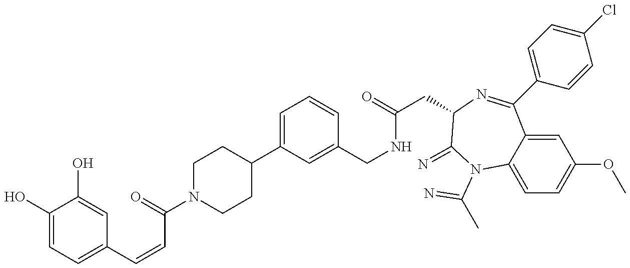 [SMILES: COc1ccc2c(c1)C(c1ccc(Cl)cc1)=N[C@@H](CC(=O)NCc1cccc(C3CCN(C(=O)/C=C\c4ccc(O)c(O)c4)CC3)c1)C(=N)N2C(C)=N] |
| 99 | 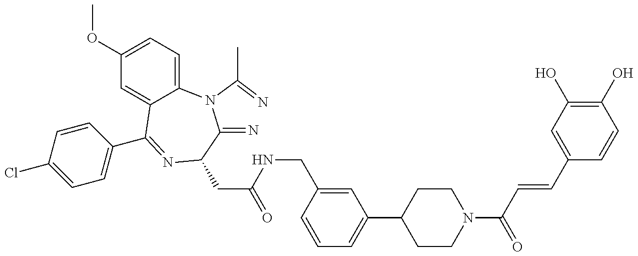 [SMILES: COc1ccc2c(c1)C(c1ccc(Cl)cc1)=N[C@@H](CC(=O)NCc1cccc(C3CCN(C(=O)/C=C/c4ccc(O)c(O)c4)CC3)c1)C(=N)N2C(C)=N] |
| 100 | 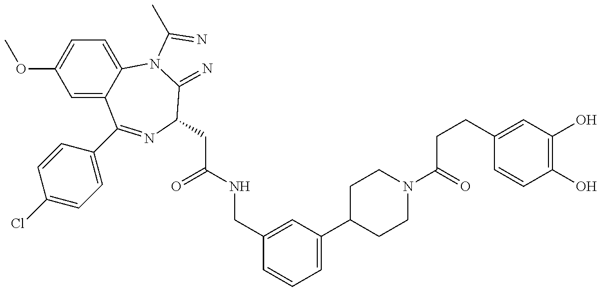 [SMILES: COc1ccc2c(c1)C(c1ccc(Cl)cc1)=N[C@@H](CC(=O)NCc1cccc(C3CCN(C(=O)CCc4ccc(O)c(O)c4)CC3)c1)C(=N)N2C(C)=N] |
| 101 | 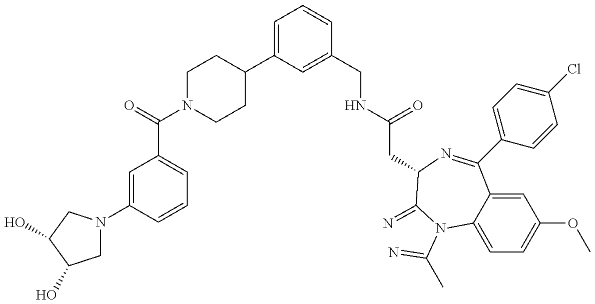 [SMILES: COc1ccc2c(c1)C(c1ccc(Cl)cc1)=N[C@@H](CC(=O)NCc1cccc(C3CCN(C(=O)c4cccc(N5C[C@@H](O)[C@@H](O)C5)c4)CC3)c1)C(=N)N2C(C)=N] |
| 102 | 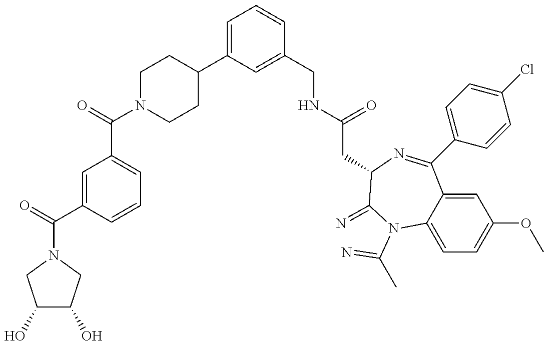 [SMILES: COc1ccc2c(c1)C(c1ccc(Cl)cc1)=N[C@@H](CC(=O)NCc1cccc(C3CCN(C(=O)c4cccc(C(=O)N5C[C@@H](O)[C@@H](O)C5)c4)CC3)c1)C(=N)N2C(C)=N] |
| 103 | 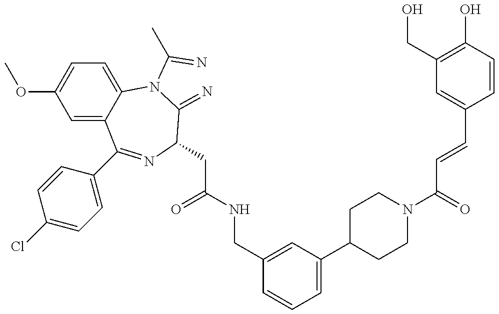 [SMILES: COc1ccc2c(c1)C(c1ccc(Cl)cc1)=N[C@@H](CC(=O)NCc1cccc(C3CCN(C(=O)/C=C/c4ccc(O)c(CO)c4)CC3)c1)C(=N)N2C(C)=N] |
| 104 | 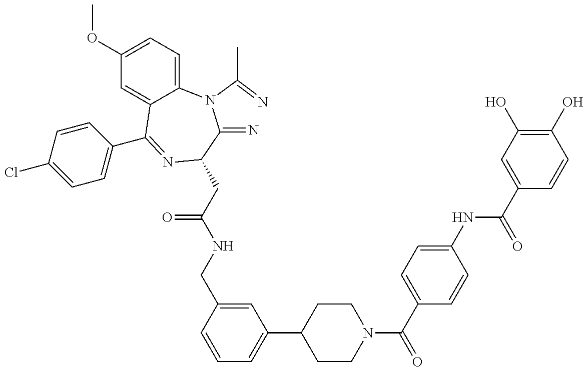 [SMILES: COc1ccc2c(c1)C(c1ccc(Cl)cc1)=N[C@@H](CC(=O)NCc1cccc(C3CCN(C(=O)c4ccc(NC(=O)c5ccc(O)c(O)c5)cc4)CC3)c1)C(=N)N2C(C)=N] |
| 105 | 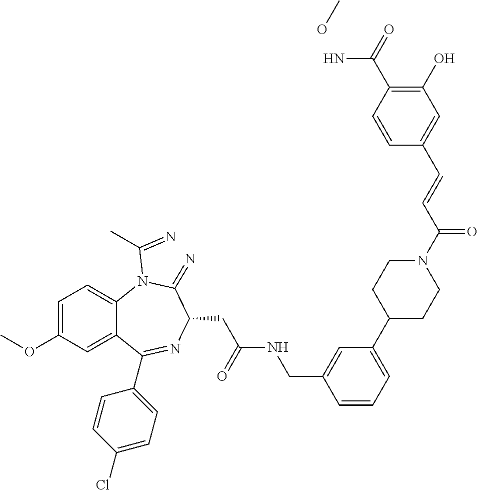 |
| 106 | 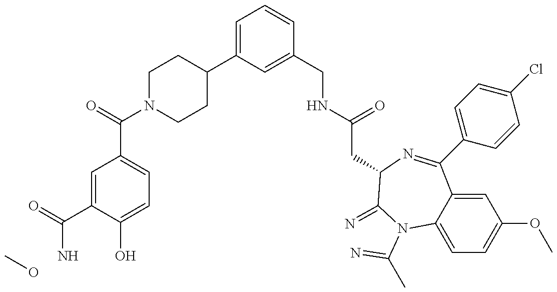 |
| 107 | 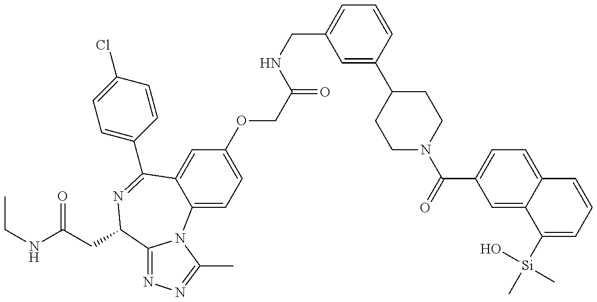 [SMILES: CCNC(=O)C[C@@H]1N=C(c2ccc(Cl)cc2)c2cc(OCC(=O)NCc3cccc(C4CCN(C(=O)c5ccc6cccc([Si](C)(C)O)c6c5)CC4)c3)ccc2-n2c(C)nnc21] |
| 108 | 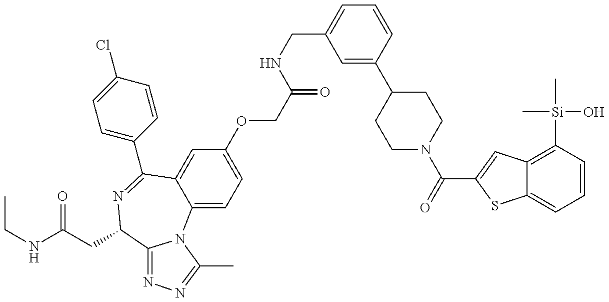 [SMILES: CCNC(=O)C[C@@H]1N=C(c2ccc(Cl)cc2)c2cc(OCC(=O)NCc3cccc(C4CCN(C(=O)c5cc6c([Si](C)(C)O)cccc6s5)CC4)c3)ccc2-n2c(C)nnc21] |
| 109 | 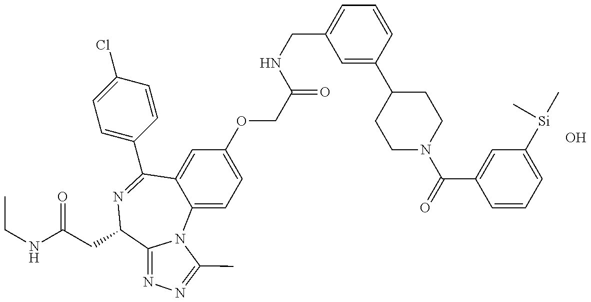 |
| 110 | 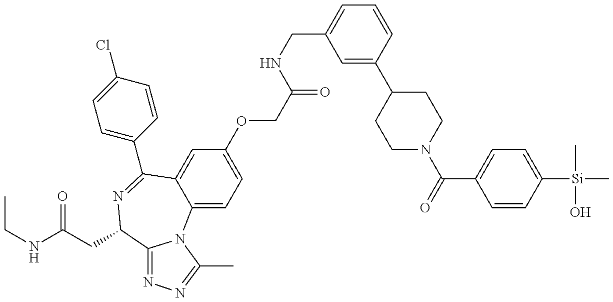 [SMILES: CCNC(=O)C[C@@H]1N=C(c2ccc(Cl)cc2)c2cc(OCC(=O)NCc3cccc(C4CCN(C(=O)c5ccc([Si](C)(C)O)cc5)CC4)c3)ccc2-n2c(C)nnc21] |
| 111 | 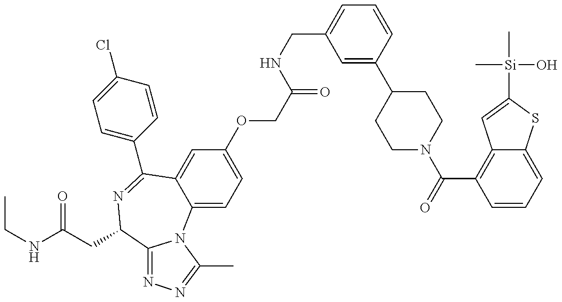 [SMILES: CCNC(=O)C[C@@H]1N=C(c2ccc(Cl)cc2)c2cc(OCC(=O)NCc3cccc(C4CCN(C(=O)c5cccc6sc([Si](C)(C)O)cc56)CC4)c3)ccc2-n2c(C)nnc21] |
| 112 | 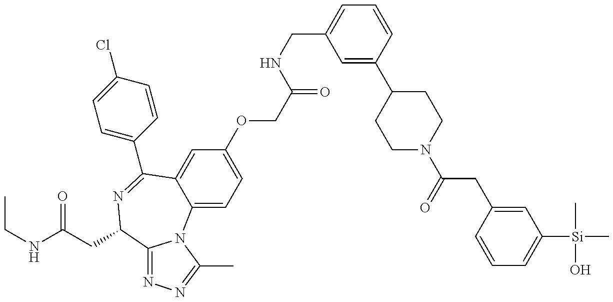 [SMILES: CCNC(=O)C[C@@H]1N=C(c2ccc(Cl)cc2)c2cc(OCC(=O)NCc3cccc(C4CCN(C(=O)Cc5cccc([Si](C)(C)O)c5)CC4)c3)ccc2-n2c(C)nnc21] |
| 113 | 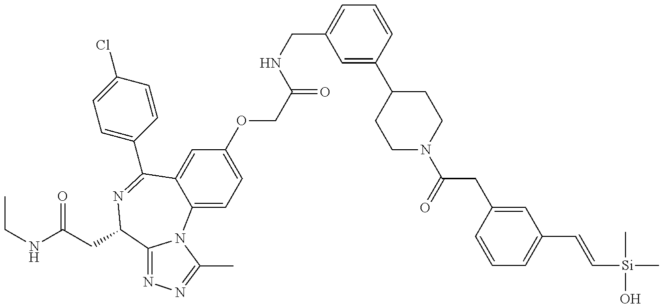 [SMILES: CCNC(=O)C[C@@H]1N=C(c2ccc(Cl)cc2)c2cc(OCC(=O)NCc3cccc(C4CCN(C(=O)Cc5cccc(/C=C/[Si](C)(C)O)c5)CC4)c3)ccc2-n2c(C)nnc21] |
| 114 | 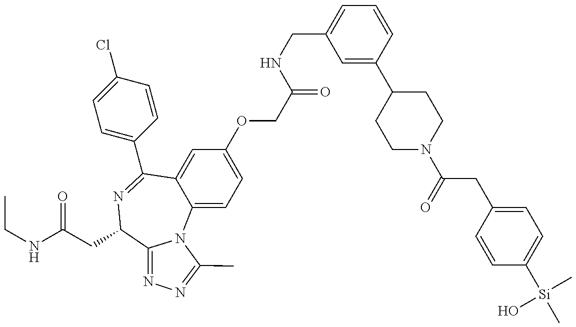 [SMILES: CCNC(=O)C[C@@H]1N=C(c2ccc(Cl)cc2)c2cc(OCC(=O)NCc3cccc(C4CCN(C(=O)Cc5ccc([Si](C)(C)O)cc5)CC4)c3)ccc2-n2c(C)nnc21] |
| 115 | 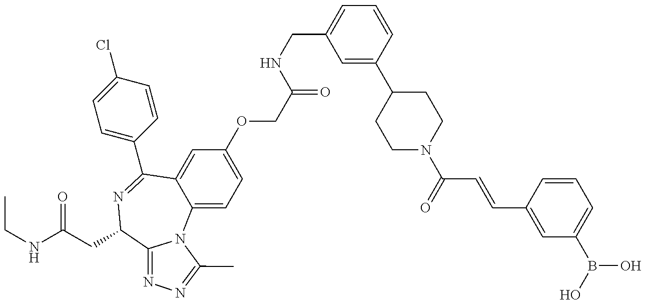 [SMILES: CCNC(=O)C[C@@H]1N=C(c2ccc(Cl)cc2)c2cc(OCC(=O)NCc3cccc(C4CCN(C(=O)/C=C/c5cccc(B(O)O)c5)CC4)c3)ccc2-n2c(C)nnc21] |
| 116 | 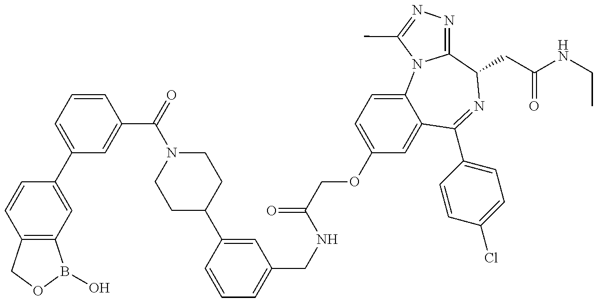 [SMILES: CCNC(=O)C[C@@H]1N=C(c2ccc(Cl)cc2)c2cc(OCC(=O)NCc3cccc(C4CCN(C(=O)c5cccc(-c6ccc7c(c6)B(O)OC7)c5)CC4)c3)ccc2-n2c(C)nnc21] |
| 117 | 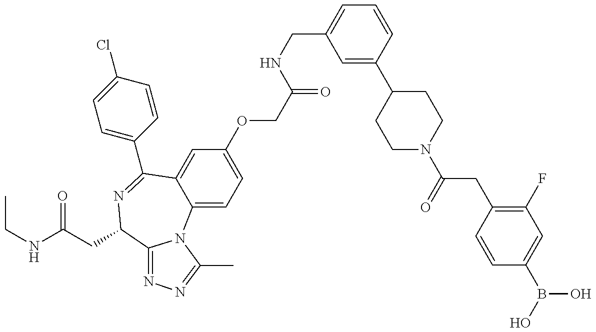 [SMILES: CCNC(=O)C[C@@H]1N=C(c2ccc(Cl)cc2)c2cc(OCC(=O)NCc3cccc(C4CCN(C(=O)Cc5ccc(B(O)O)cc5F)CC4)c3)ccc2-n2c(C)nnc21] |
| 118 | 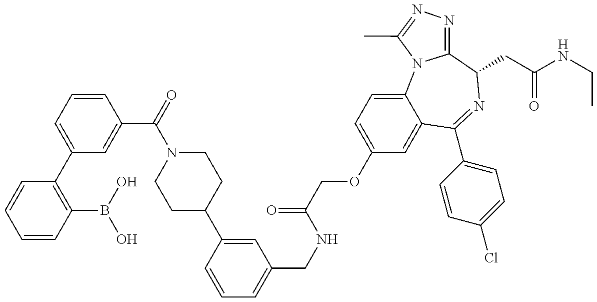 [SMILES: CCNC(=O)C[C@@H]1N=C(c2ccc(Cl)cc2)c2cc(OCC(=O)NCc3cccc(C4CCN(C(=O)c5cccc(-c6ccccc6B(O)O)c5)CC4)c3)ccc2-n2c(C)nnc21] |
| 119 | 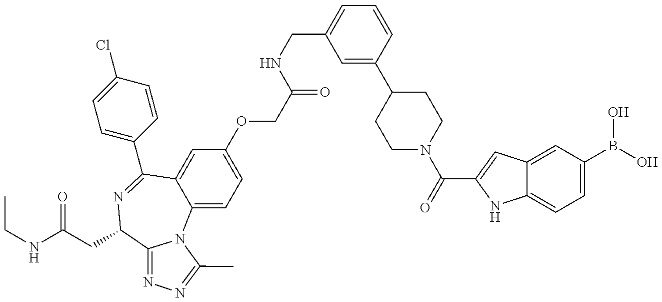 [SMILES: CCNC(=O)C[C@@H]1N=C(c2ccc(Cl)cc2)c2cc(OCC(=O)NCc3cccc(C4CCN(C(=O)c5cc6cc(B(O)O)ccc6[nH]5)CC4)c3)ccc2-n2c(C)nnc21] |
| 120 | 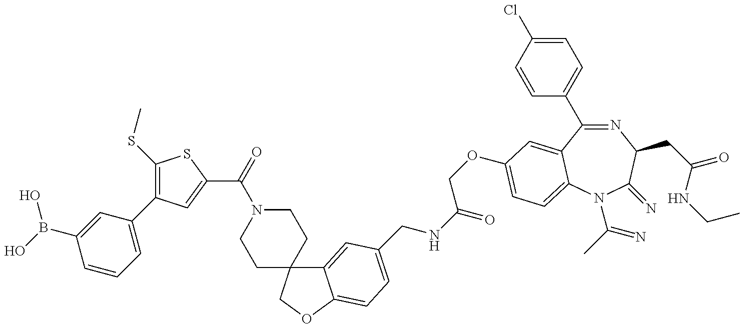 [SMILES: CCNC(=O)C[C@@H]1N=C(c2ccc(Cl)cc2)c2cc(OCC(=O)NCc3ccc4c(c3)C3(CCN(C(=O)c5cc(-c6cccc(B(O)O)c6)c(SC)s5)CC3)CO4)ccc2N(C(C)=N)C1=N] |
| 121 | 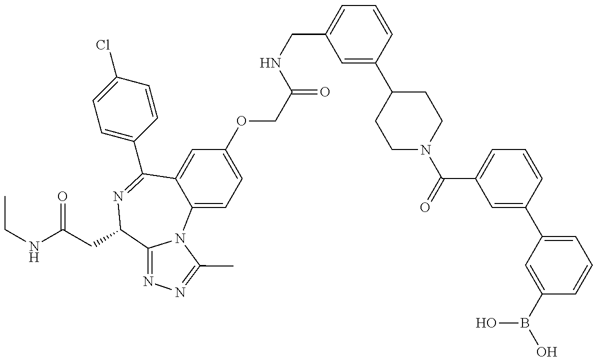 [SMILES: CCNC(=O)C[C@@H]1N=C(c2ccc(Cl)cc2)c2cc(OCC(=O)NCc3cccc(C4CCN(C(=O)c5cccc(-c6cccc(B(O)O)c6)c5)CC4)c3)ccc2-n2c(C)nnc21] |
| 122 | 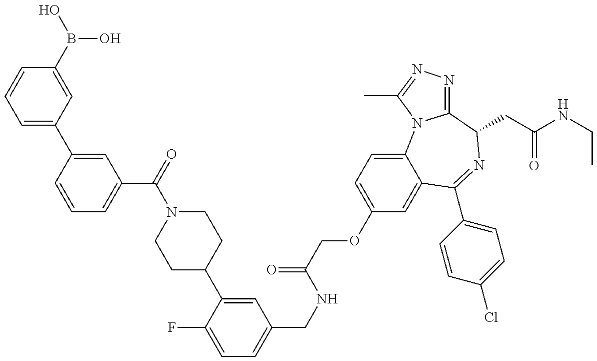 [SMILES: CCNC(=O)C[C@@H]1N=C(c2ccc(Cl)cc2)c2cc(OCC(=O)NCc3ccc(F)c(C4CCN(C(=O)c5cccc(-c6cccc(B(O)O)c6)c5)CC4)c3)ccc2-n2c(C)nnc21] |
| 123 | 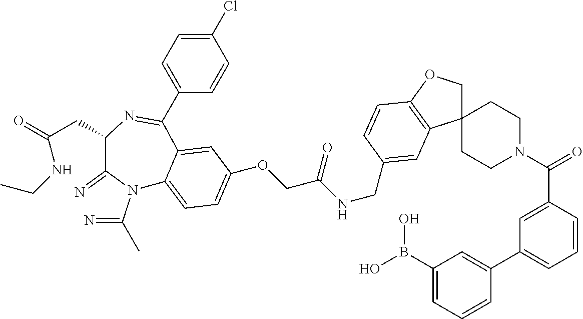 [SMILES: CCNC(=O)C[C@@H]1N=C(c2ccc(Cl)cc2)c2cc(OCC(=O)NCc3ccc4c(c3)C3(CCN(C(=O)c5cccc(-c6cccc(B(O)O)c6)c5)CC3)CO4)ccc2N(C(C)=N)C1=N] |
| 124 | 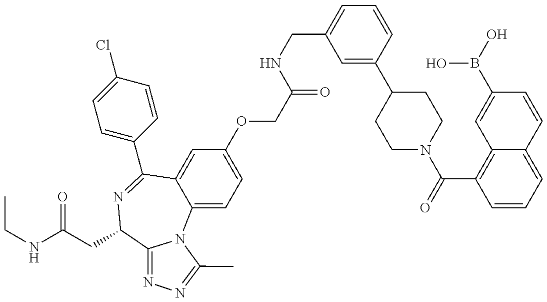 [SMILES: CCNC(=O)C[C@@H]1N=C(c2ccc(Cl)cc2)c2cc(OCC(=O)NCc3cccc(C4CCN(C(=O)c5cccc6ccc(B(O)O)cc56)CC4)c3)ccc2-n2c(C)nnc21] |
| 125 | 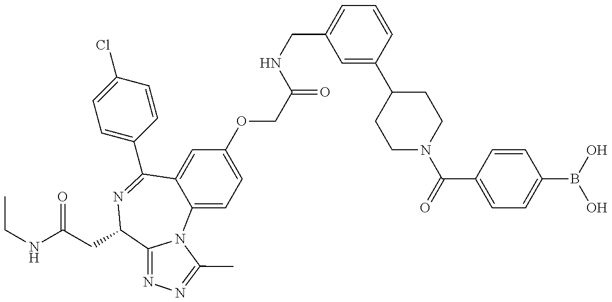 [SMILES: CCNC(=O)C[C@@H]1N=C(c2ccc(Cl)cc2)c2cc(OCC(=O)NCc3cccc(C4CCN(C(=O)c5ccc(B(O)O)cc5)CC4)c3)ccc2-n2c(C)nnc21] |
| 126 | 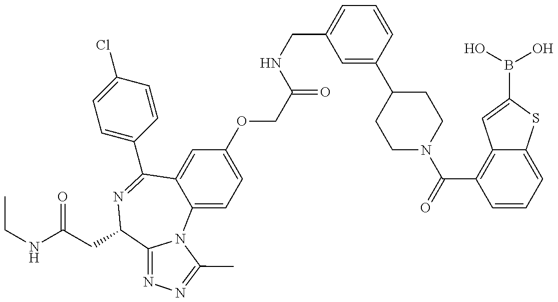 [SMILES: CCNC(=O)C[C@@H]1N=C(c2ccc(Cl)cc2)c2cc(OCC(=O)NCc3cccc(C4CCN(C(=O)c5cccc6sc(B(O)O)cc56)CC4)c3)ccc2-n2c(C)nnc21] |
| 127 | 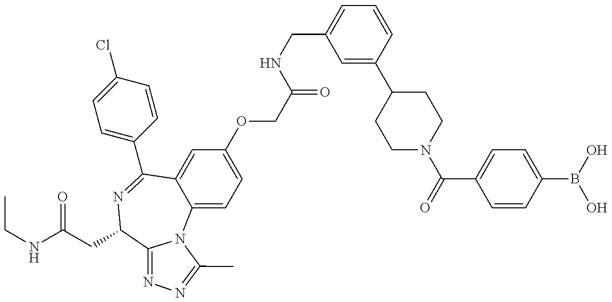 [SMILES: CCNC(=O)C[C@@H]1N=C(c2ccc(Cl)cc2)c2cc(OCC(=O)NCc3cccc(C4CCN(C(=O)c5ccc(B(O)O)cc5)CC4)c3)ccc2-n2c(C)nnc21] |
| 128 | 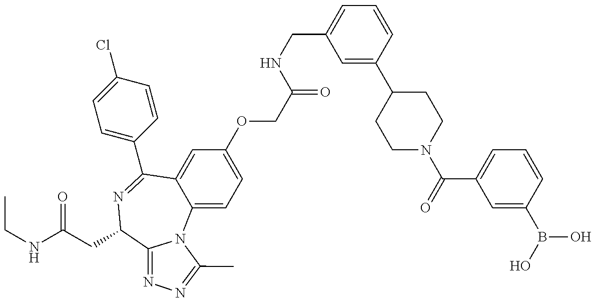 [SMILES: CCNC(=O)C[C@@H]1N=C(c2ccc(Cl)cc2)c2cc(OCC(=O)NCc3cccc(C4CCN(C(=O)c5cccc(B(O)O)c5)CC4)c3)ccc2-n2c(C)nnc21] |
| 129 | 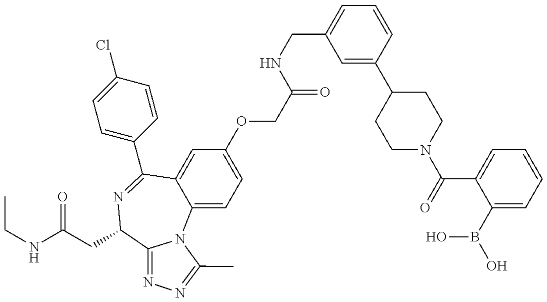 [SMILES: CCNC(=O)C[C@@H]1N=C(c2ccc(Cl)cc2)c2cc(OCC(=O)NCc3cccc(C4CCN(C(=O)c5ccccc5B(O)O)CC4)c3)ccc2-n2c(C)nnc21] |
| 130 | 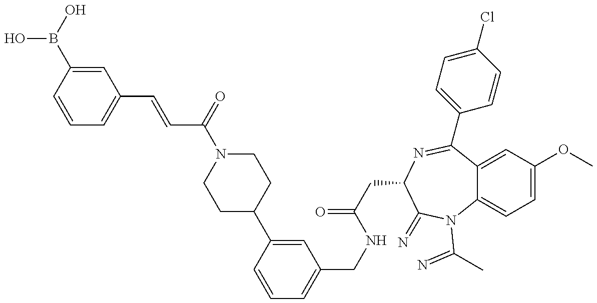 [SMILES: COc1ccc2c(c1)C(c1ccc(Cl)cc1)=N[C@@H](CC(=O)NCc1cccc(C3CCN(C(=O)/C=C/c4cccc(B(O)O)c4)CC3)c1)C(=N)N2C(C)=N] |
| 131 | 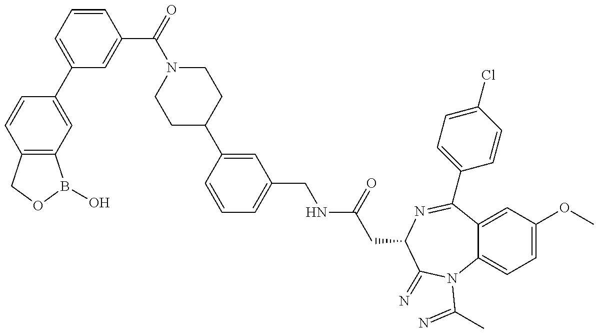 [SMILES: COc1ccc2c(c1)C(c1ccc(Cl)cc1)=N[C@@H](CC(=O)NCc1cccc(C3CCN(C(=O)c4cccc(-c5ccc6c(c5)B(O)OC6)c4)CC3)c1)C(=N)N2C(C)=N] |
| 132 | 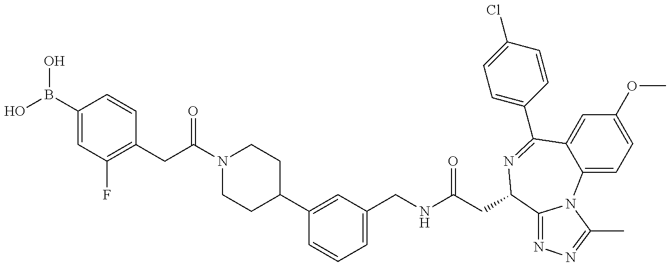 [SMILES: COc1ccc2c(c1)C(c1ccc(Cl)cc1)=N[C@@H](CC(=O)NCc1cccc(C3CCN(C(=O)Cc4ccc(B(O)O)cc4F)CC3)c1)c1nnc(C)n1-2] |
| 133 | 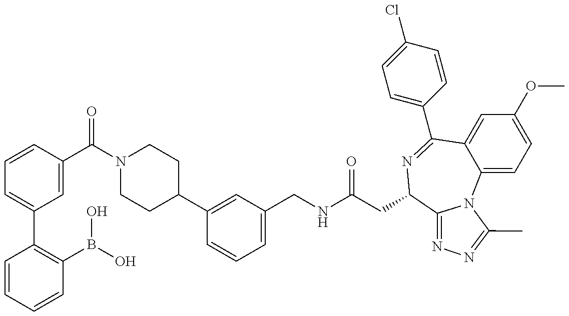 [SMILES: COc1ccc2c(c1)C(c1ccc(Cl)cc1)=N[C@@H](CC(=O)NCc1cccc(C3CCN(C(=O)c4cccc(-c5ccccc5B(O)O)c4)CC3)c1)c1nnc(C)n1-2] |
| 134 | 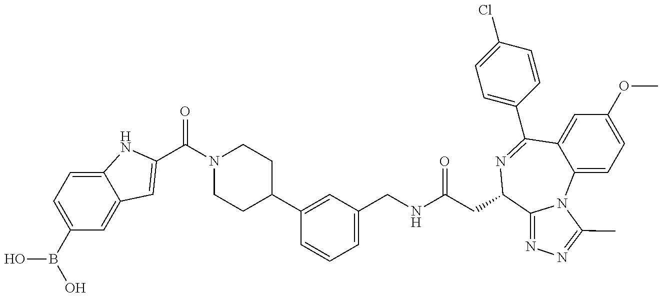 [SMILES: COc1ccc2c(c1)C(c1ccc(Cl)cc1)=N[C@@H](CC(=O)NCc1cccc(C3CCN(C(=O)c4cc5cc(B(O)O)ccc5[nH]4)CC3)c1)c1nnc(C)n1-2] |
| 135 | 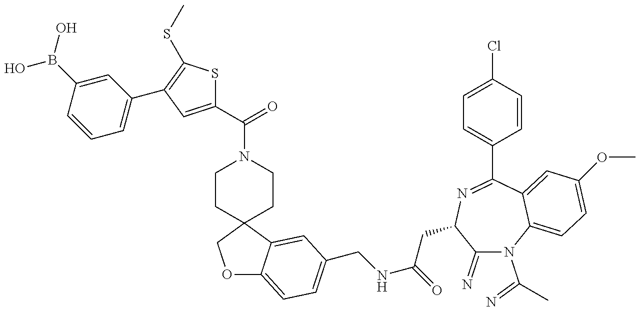 [SMILES: COc1ccc2c(c1)C(c1ccc(Cl)cc1)=N[C@@H](CC(=O)NCc1ccc3c(c1)C1(CCN(C(=O)c4cc(-c5cccc(B(O)O)c5)c(SC)s4)CC1)CO3)C(=N)N2C(C)=N] |
| 136 | 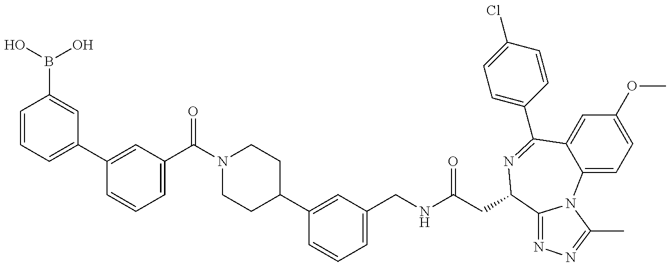 [SMILES: COc1ccc2c(c1)C(c1ccc(Cl)cc1)=N[C@@H](CC(=O)NCc1cccc(C3CCN(C(=O)c4cccc(-c5cccc(B(O)O)c5)c4)CC3)c1)c1nnc(C)n1-2] |
| 137 | 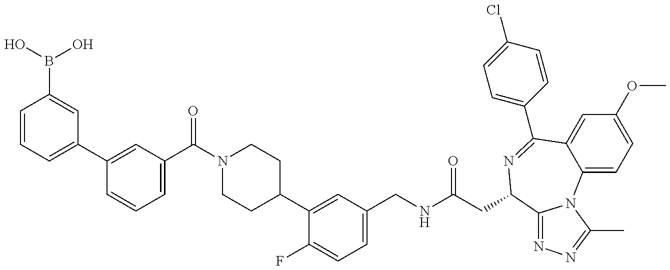 [SMILES: COc1ccc2c(c1)C(c1ccc(Cl)cc1)=N[C@@H](CC(=O)NCc1ccc(F)c(C3CCN(C(=O)c4cccc(-c5cccc(B(O)O)c5)c4)CC3)c1)c1nnc(C)n1-2] |
| 138 | 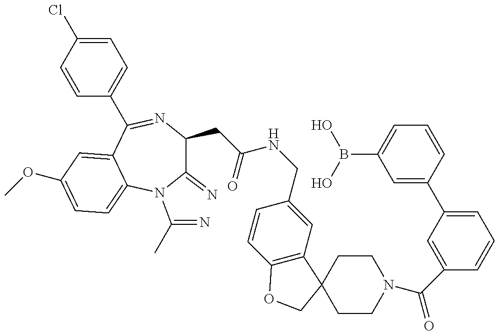 [SMILES: COc1ccc2c(c1)C(c1ccc(Cl)cc1)=N[C@@H](CC(=O)NCc1ccc3c(c1)C1(CCN(C(=O)c4cccc(-c5cccc(B(O)O)c5)c4)CC1)CO3)C(=N)N2C(C)=N] |
| 139 | 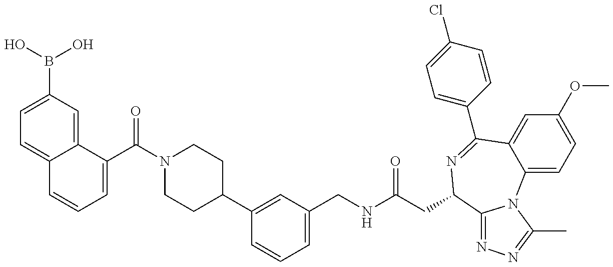 [SMILES: COc1ccc2c(c1)C(c1ccc(Cl)cc1)=N[C@@H](CC(=O)NCc1cccc(C3CCN(C(=O)c4cccc5ccc(B(O)O)cc45)CC3)c1)c1nnc(C)n1-2] |
| 140 | 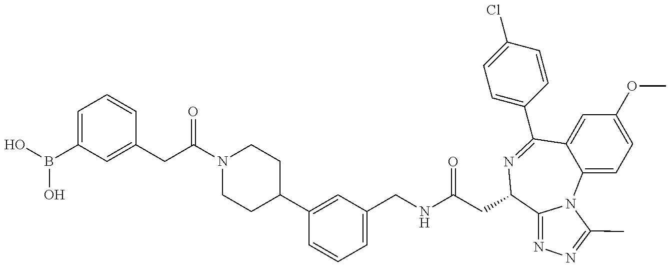 [SMILES: COc1ccc2c(c1)C(c1ccc(Cl)cc1)=N[C@@H](CC(=O)NCc1cccc(C3CCN(C(=O)Cc4cccc(B(O)O)c4)CC3)c1)c1nnc(C)n1-2] |
| 141 | 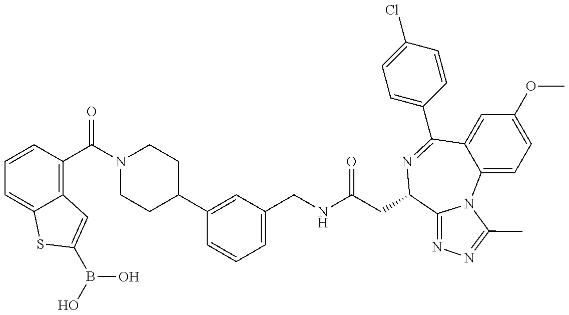 [SMILES: COc1ccc2c(c1)C(c1ccc(Cl)cc1)=N[C@@H](CC(=O)NCc1cccc(C3CCN(C(=O)c4cccc5sc(B(O)O)cc45)CC3)c1)c1nnc(C)n1-2] |
| 142 | 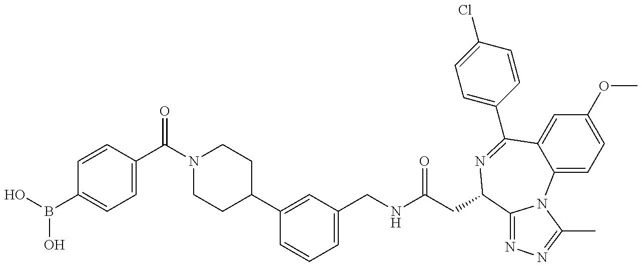 [SMILES: COc1ccc2c(c1)C(c1ccc(Cl)cc1)=N[C@@H](CC(=O)NCc1cccc(C3CCN(C(=O)c4ccc(B(O)O)cc4)CC3)c1)c1nnc(C)n1-2] |
| 143 | 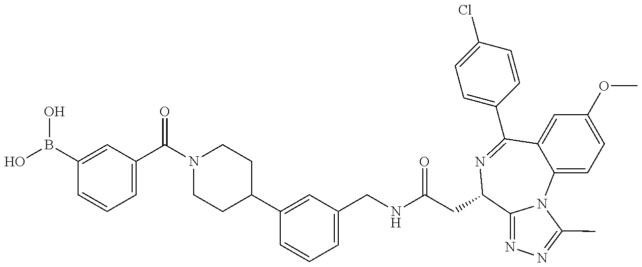 [SMILES: COc1ccc2c(c1)C(c1ccc(Cl)cc1)=N[C@@H](CC(=O)NCc1cccc(C3CCN(C(=O)c4cccc(B(O)O)c4)CC3)c1)c1nnc(C)n1-2] |
| 144 | 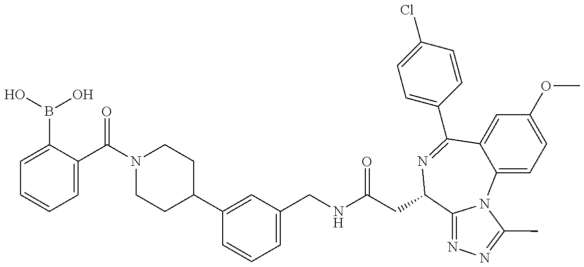 [SMILES: COc1ccc2c(c1)C(c1ccc(Cl)cc1)=N[C@@H](CC(=O)NCc1cccc(C3CCN(C(=O)c4ccccc4B(O)O)CC3)c1)c1nnc(C)n1-2] |

### Examples 145-314

Table X recites monomers which may form a dimer with a second monomer. The monomer examples in Table X, having boronic acid, diol or silanol linkers (Z), correspond to the structures in Table D.

TABLE X

| | Compound Structure |
|---|---|
| No. | (e.g., X—Y—Z) |
| 146 | 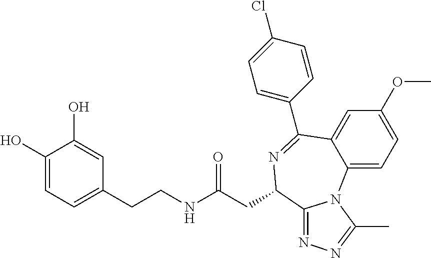 [SMILES: COc1ccc2c(c1)C(c1ccc(Cl)cc1)=N[C@@H](CC(=O)NCCc1ccc(O)c(O)c1)c1nnc(C)n1-2] |
| 147 | 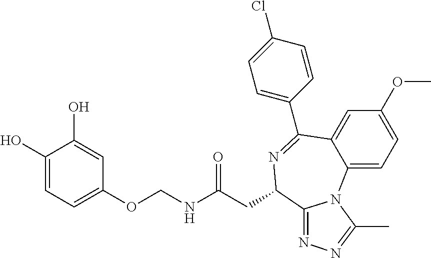 [SMILES: COc1ccc2c(c1)C(c1ccc(Cl)cc1)=N[C@@H](CC(=O)NCOc1ccc(O)c(O)c1)c1nnc(C)n1-2] |
| 148 | 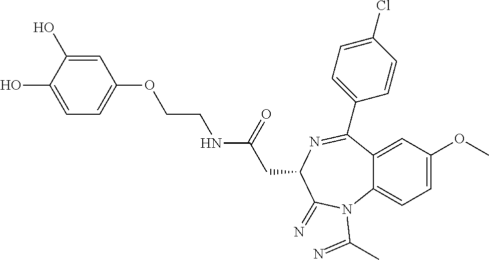 [SMILES: COc1ccc2c(c1)C(c1ccc(Cl)cc1)=N[C@@H](CC(=O)NCCOc1ccc(O)c(O)c1)C(=N)N2C(C)=N] |
| 149 | 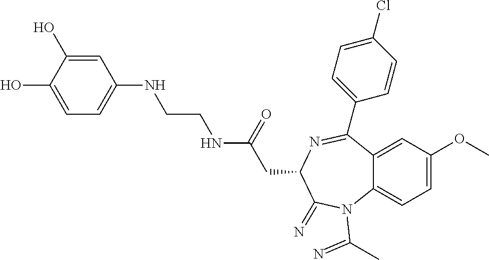 [SMILES: COc1ccc2c(c1)C(c1ccc(Cl)cc1)=N[C@@H](CC(=O)NCCNc1ccc(O)c(O)c1)C(=N)N2C(C)=N] |
| 150 | 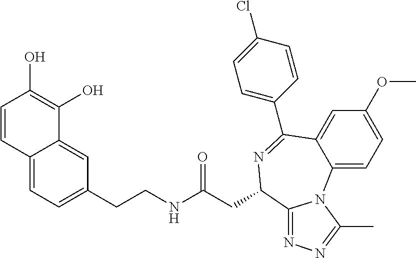 [SMILES: COc1ccc2c(c1)C(c1ccc(Cl)cc1)=N[C@@H](CC(=O)NCCc1ccc3ccc(O)c(O)c3c1)c1nnc(C)n1-2] |
| 151 | 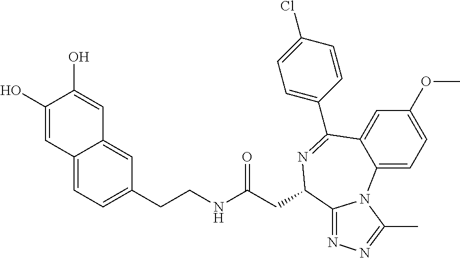 [SMILES: COc1ccc2c(c1)C(c1ccc(Cl)cc1)=N[C@@H](CC(=O)NCCc1ccc3cc(O)c(O)cc3c1)c1nnc(C)n1-2] |
| 152 | 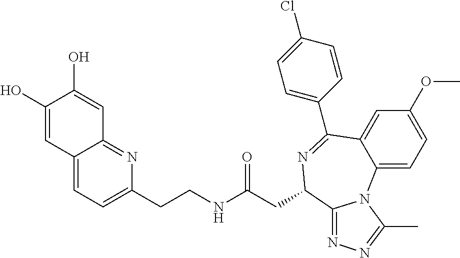 [SMILES: COc1ccc2c(c1)C(c1ccc(Cl)cc1)=N[C@@H](CC(=O)NCCc1ccc3cc(O)c(O)cc3n1)c1nnc(C)n1-2] |
| 153 | 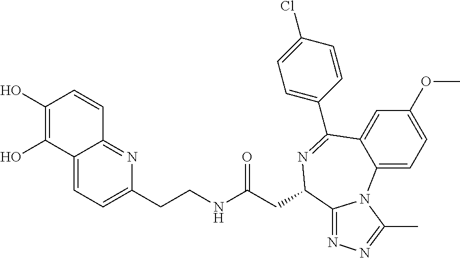 [SMILES: COc1ccc2c(c1)C(c1ccc(Cl)cc1)=N[C@@H](CC(=O)NCCc1ccc3c(O)c(O)ccc3n1)c1nnc(C)n1-2] |
| 154 | 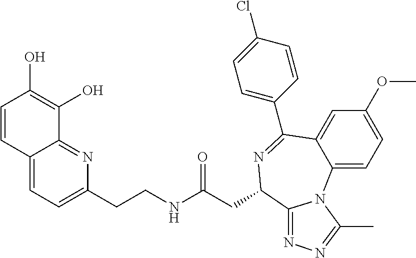 [SMILES: COc1ccc2c(c1)C(c1ccc(Cl)cc1)=N[C@@H](CC(=O)NCCc1ccc3ccc(O)c(O)c3n1)c1nnc(C)n1-2] |
| 155 | 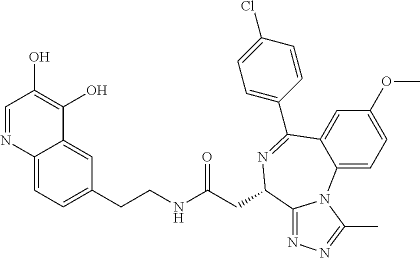 [SMILES: COc1ccc2c(c1)C(c1ccc(Cl)cc1)=N[C@@H](CC(=O)NCCc1ccc3ncc(O)c(O)c3c1)c1nnc(C)n1-2] |
| 156 | 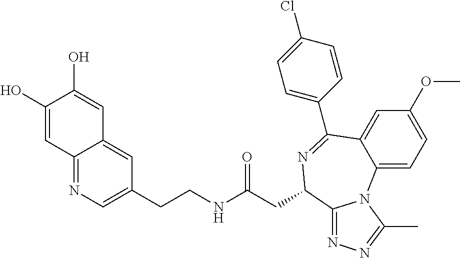 [SMILES: COc1ccc2c(c1)C(c1ccc(Cl)cc1)=N[C@@H](CC(=O)NCCc1cnc3cc(O)c(O)cc3c1)c1nnc(C)n1-2] |
| 157 | 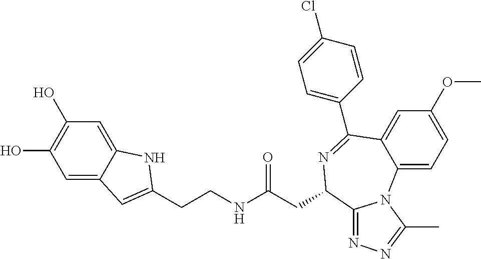 [SMILES: COc1ccc2c(c1)C(c1ccc(Cl)cc1)=N[C@@H](CC(=O)NCCc1cc3cc(O)c(O)cc3[nH]1)c1nnc(C)n1-2] |
| 158 | 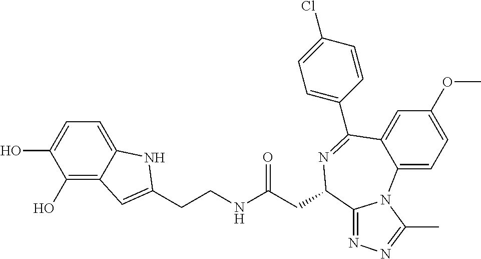 [SMILES: COc1ccc2c(c1)C(c1ccc(Cl)cc1)=N[C@@H](CC(=O)NCCc1cc3c(O)c(O)ccc3[nH]1)c1nnc(C)n1-2] |
| 159 | 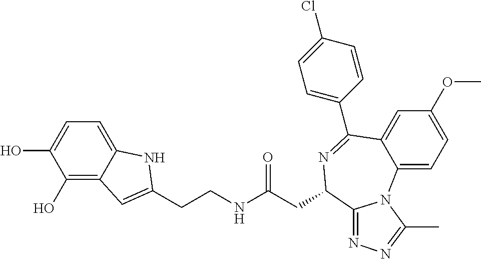 [SMILES: COc1ccc2c(c1)C(c1ccc(Cl)cc1)=N[C@@H](CC(=O)NCCc1cc3c(O)c(O)ccc3[nH]1)c1nnc(C)n1-2] |
| 160 | 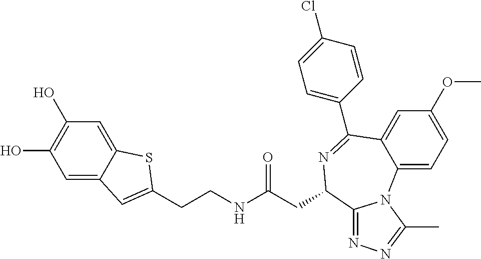 [SMILES: COc1ccc2c(c1)C(c1ccc(Cl)cc1)=N[C@@H](CC(=O)NCCc1cc3cc(O)c(O)cc3s1)c1nnc(C)n1-2] |
| 161 | 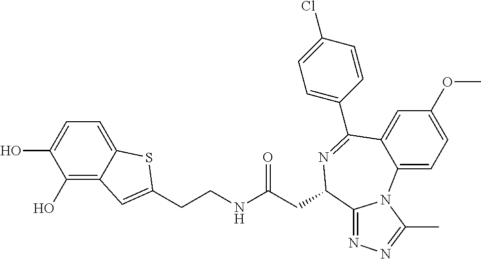 [SMILES: COc1ccc2c(c1)C(c1ccc(Cl)cc1)=N[C@@H](CC(=O)NCCc1cc3c(O)c(O)ccc3s1)c1nnc(C)n1-2] |
| 162 | 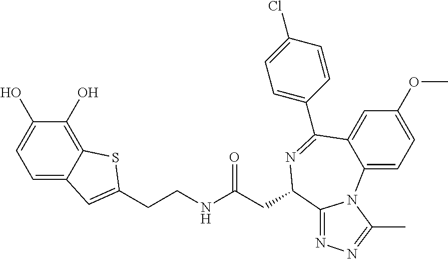 [SMILES: COc1ccc2c(c1)C(c1ccc(Cl)cc1)=N[C@@H](CC(=O)NCCc1cc3ccc(O)c(O)c3s1)c1nnc(C)n1-2] |
| 163 | 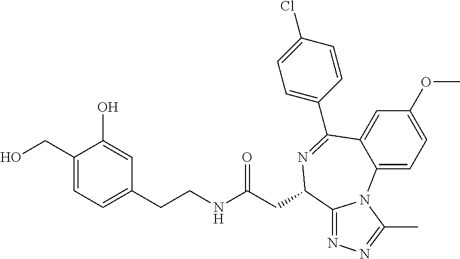 [SMILES: COc1ccc2c(c1)C(c1ccc(Cl)cc1)=N[C@@H](CC(=O)NCCc1ccc(CO)c(O)c1)c1nnc(C)n1-2] |
| 164 | 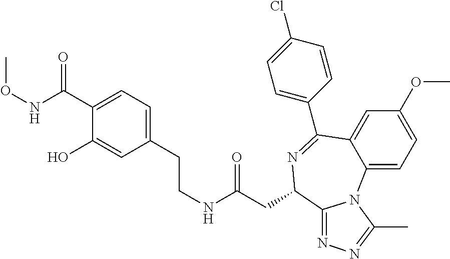 [SMILES: CONC(=O)c1ccc(CCNC(=O)C[C@@H]2N=C(c3ccc(Cl)cc3)c3cc(OC)ccc3-n3c(C)nnc32)cc1O] |
| 165 | 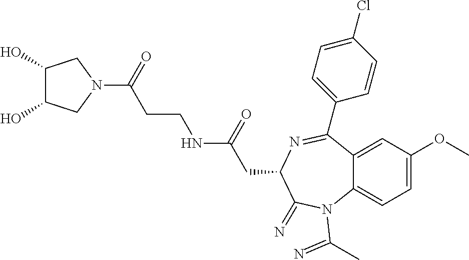 [SMILES: COc1ccc2c(c1)C(c1ccc(Cl)cc1)=N[C@@H](CC(=O)NCCC(=O)N1C[C@@H](O)[C@@H](O)C1)C(=N)N2C(C)=N] |
| 166 | 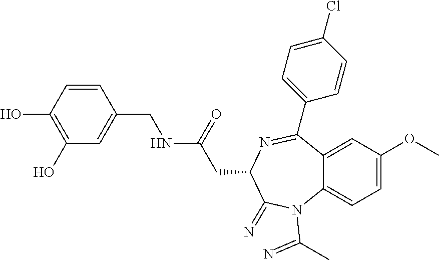 [SMILES: COc1ccc2c(c1)C(c1ccc(Cl)cc1)=N[C@@H](CC(=O)NCc1ccc(O)c(O)c1)C(=N)N2C(C)=N] |
| 167 | 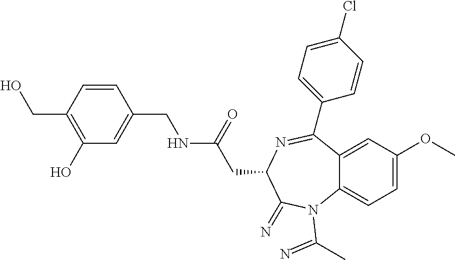 [SMILES: COc1ccc2c(c1)C(c1ccc(Cl)cc1)=N[C@@H](CC(=O)NCc1ccc(CO)c(O)c1)C(=N)N2C(C)=N] |
| 168 | 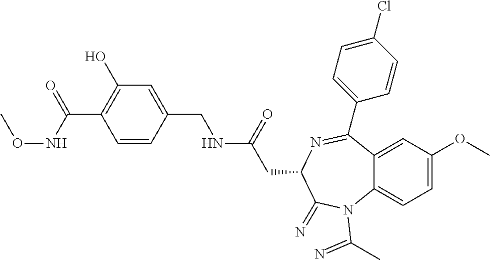 |
| 169 | 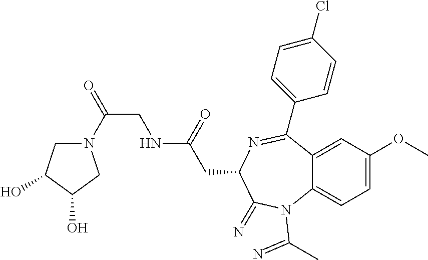 [SMILES: COc1ccc2c(c1)C(c1ccc(Cl)cc1)=N[C@@H](CC(=O)NCC(=O)N1C[C@@H](O)[C@@H](O)C1)C(=N)N2C(C)=N] |
| 170 | 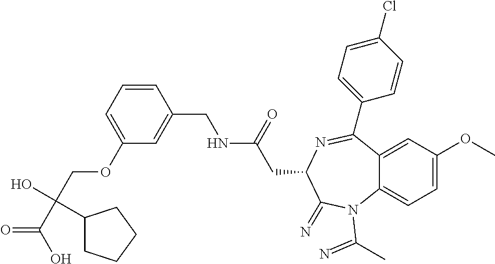 [SMILES: COc1ccc2c(c1)C(c1ccc(Cl)cc1)=N[C@@H](CC(=O)NCc1cccc(OCC(O)(C(=O)O)C3CCCC3)c1)C(=N)N2C(C)=N] |
| 171 | 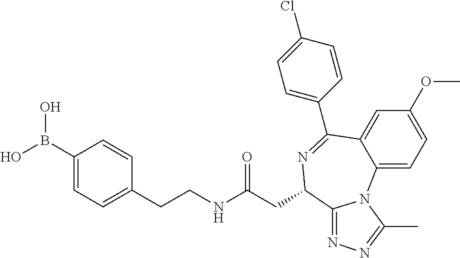 [SMILES: COc1ccc2c(c1)C(c1ccc(Cl)cc1)=N[C@@H](CC(=O)NCCc1ccc(B(O)O)cc1)c1nnc(C)n1-2] |
| 172 | 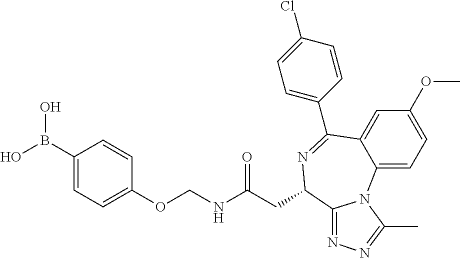 [SMILES: COc1ccc2c(c1)C(c1ccc(Cl)cc1)=N[C@@H](CC(=O)NCOc1ccc(B(O)O)cc1)c1nnc(C)n1-2] |
| 173 | 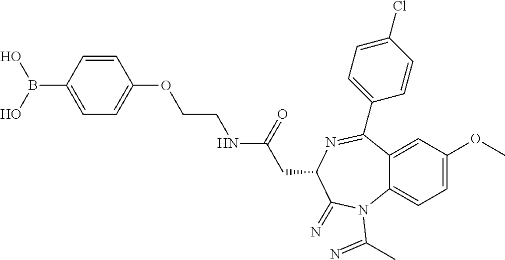 [SMILES: COc1ccc2c(c1)C(c1ccc(Cl)cc1)=N[C@@H](CC(=O)NCCOc1ccc(B(O)O)cc1)C(=N)N2C(C)=N] |
| 174 | 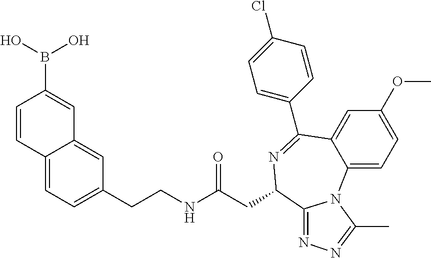 [SMILES: COc1ccc2c(c1)C(c1ccc(Cl)cc1)=N[C@@H](CC(=O)NCCc1ccc3ccc(B(O)O)cc3c1)c1nnc(C)n1-2] |
| 175 | 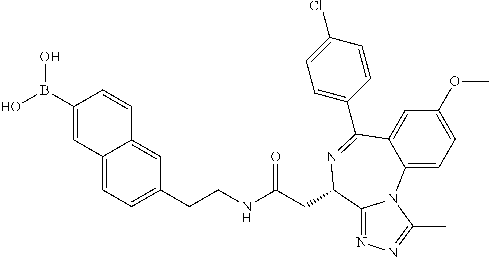 [SMILES: COc1ccc2c(c1)C(c1ccc(Cl)cc1)=N[C@@H](CC(=O)NCCc1ccc3cc(B(O)O)ccc3c1)c1nnc(C)n1-2] |
| 176 | 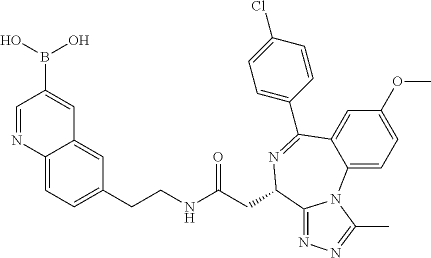 [SMILES: COc1ccc2c(c1)C(c1ccc(Cl)cc1)=N[C@@H](CC(=O)NCCc1ccc3ncc(B(O)O)cc3c1)c1nnc(C)n1-2] |
| 177 | 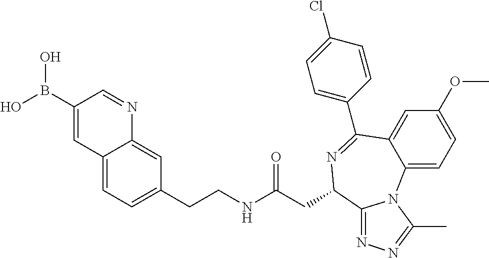 [SMILES: COc1ccc2c(c1)C(c1ccc(Cl)cc1)=N[C@@H](CC(=O)NCCc1ccc3cc(B(O)O)cnc3c1)c1nnc(C)n1-2] |
| 178 | 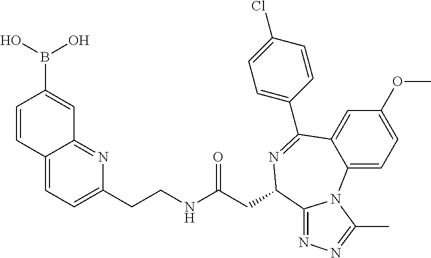 [SMILES: COc1ccc2c(c1)C(c1ccc(Cl)cc1)=N[C@@H](CC(=O)NCCc1ccc3ccc(B(O)O)cc3n1)c1nnc(C)n1-2] |
| 179 | 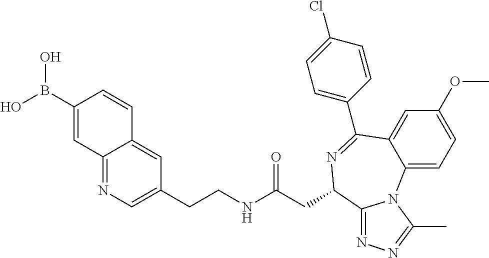 [SMILES: COc1ccc2c(c1)C(c1ccc(Cl)cc1)=N[C@@H](CC(=O)NCCc1cnc3cc(B(O)O)ccc3c1)c1nnc(C)n1-2] |
| 180 | 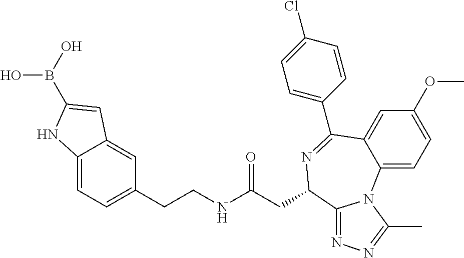 [SMILES: COc1ccc2c(c1)C(c1ccc(Cl)cc1)=N[C@@H](CC(=O)NCCc1ccc3[nH]c(B(O)O)cc3c1)c1nnc(C)n1-2] |
| 181 | 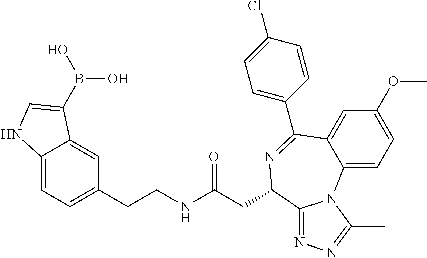 [SMILES: COc1ccc2c(c1)C(c1ccc(Cl)cc1)=N[C@@H](CC(=O)NCCc1ccc3[nH]cc(B(O)O)c3c1)c1nnc(C)n1-2] |
| 182 | 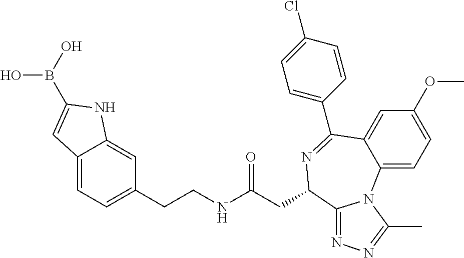 [SMILES: COc1ccc2c(c1)C(c1ccc(Cl)cc1)=N[C@@H](CC(=O)NCCc1ccc3cc(B(O)O)[nH]c3c1)c1nnc(C)n1-2] |
| 183 | 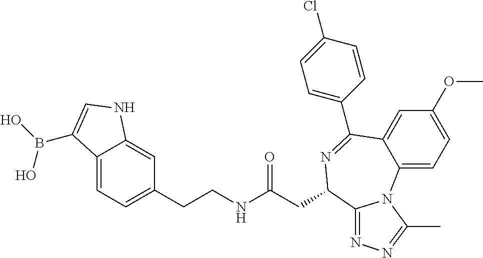 [SMILES: COc1ccc2c(c1)C(c1ccc(Cl)cc1)=N[C@@H](CC(=O)NCCc1ccc3c(B(O)O)c[nH]c3c1)c1nnc(C)n1-2] |
| 184 | 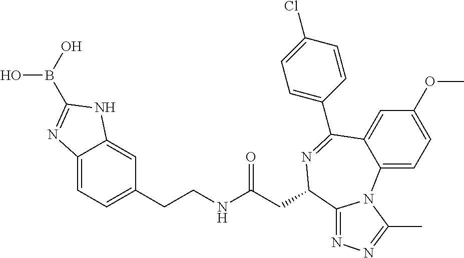 [SMILES: COc1ccc2c(c1)C(c1ccc(Cl)cc1)=N[C@@H](CC(=O)NCCc1ccc3nc(B(O)O)[nH]c3c1)c1nnc(C)n1-2] |
| 185 | 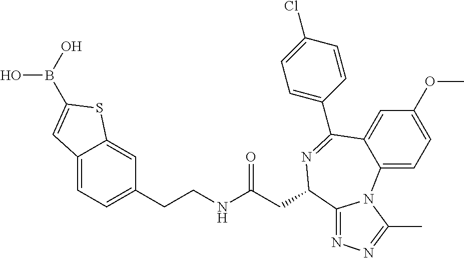 [SMILES: COc1ccc2c(c1)C(c1ccc(Cl)cc1)=N[C@@H](CC(=O)NCCc1ccc3cc(B(O)O)sc3c1)c1nnc(C)n1-2] |
| 186 | 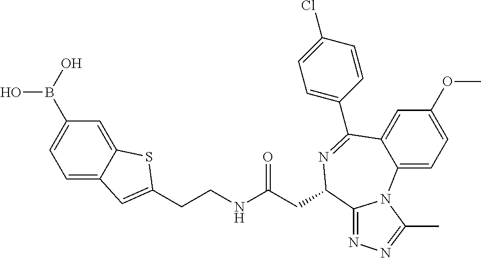 [SMILES: COc1ccc2c(c1)C(c1ccc(Cl)cc1)=N[C@@H](CC(=O)NCCc1cc3ccc(B(O)O)cc3s1)c1nnc(C)n1-2] |
| 187 | 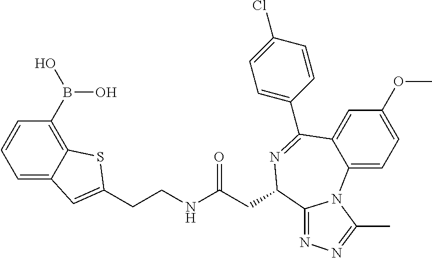 [SMILES: COc1ccc2c(c1)C(c1ccc(Cl)cc1)=N[C@@H](CC(=O)NCCc1cc3cccc(B(O)O)c3s1)c1nnc(C)n1-2] |
| 188 | 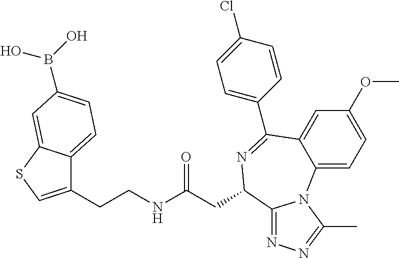 [SMILES: COc1ccc2c(c1)C(c1ccc(Cl)cc1)=N[C@@H](CC(=O)NCCc1csc3cc(B(O)O)ccc13)c1nnc(C)n1-2] |
| 189 | 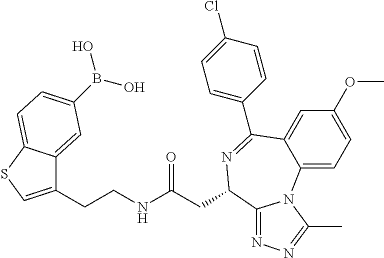 [SMILES: COc1ccc2c(c1)C(c1ccc(Cl)cc1)=N[C@@H](CC(=O)NCCc1csc3ccc(B(O)O)cc13)c1nnc(C)n1-2] |
| 190 | 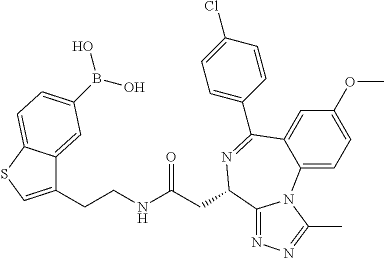 [SMILES: COc1ccc2c(c1)C(c1ccc(Cl)cc1)=N[C@@H](CC(=O)NCCc1csc3ccc(B(O)O)cc13)c1nnc(C)n1-2] |
| 191 | 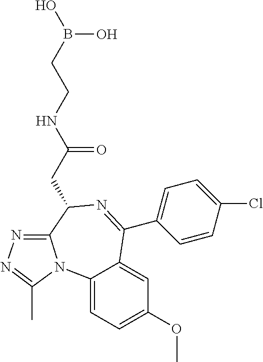 [SMILES: COc1ccc2c(c1)C(c1ccc(Cl)cc1)=N[C@@H](CC(=O)NCCB(O)O)c1nnc(C)n1-2] |
| 192 | 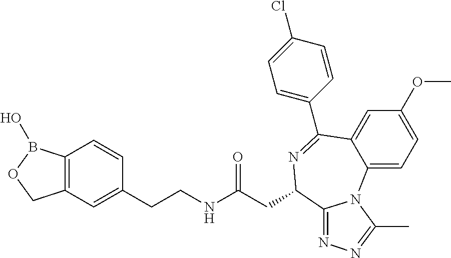 [SMILES: COc1ccc2c(c1)C(c1ccc(Cl)cc1)=N[C@@H](CC(=O)NCCc1ccc3c(c1)COB3O)c1nnc(C)n1-2] |
| 193 | 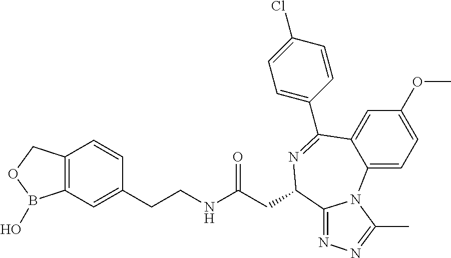 [SMILES: COc1ccc2c(c1)C(c1ccc(Cl)cc1)=N[C@@H](CC(=O)NCCc1ccc3c(c1)B(O)OC3)c1nnc(C)n1-2] |
| 194 | 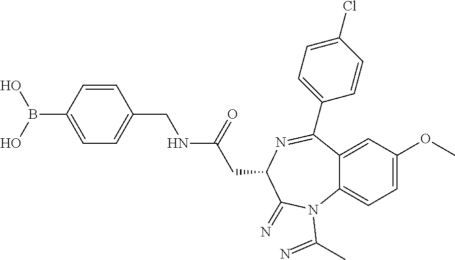 [SMILES: COc1ccc2c(c1)C(c1ccc(Cl)cc1)=N[C@@H](CC(=O)NCc1ccc(B(O)O)cc1)C(=N)N2C(C)=N] |
| 195 | 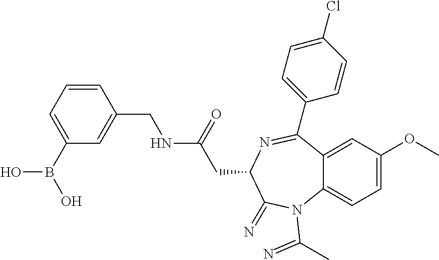 [SMILES: COc1ccc2c(c1)C(c1ccc(Cl)cc1)=N[C@@H](CC(=O)NCc1cccc(B(O)O)c1)C(=N)N2C(C)=N] |
| 196 | 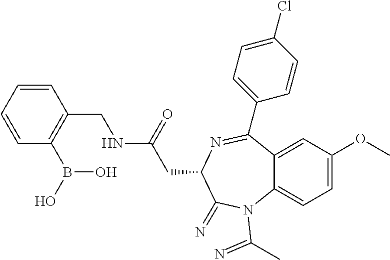 [SMILES: COc1ccc2c(c1)C(c1ccc(Cl)cc1)=N[C@@H](CC(=O)NCc1ccccc1B(O)O)C(=N)N2C(C)=N] |
| 197 | 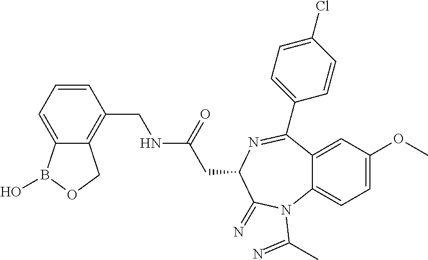 [SMILES: COc1ccc2c(c1)C(c1ccc(Cl)cc1)=N[C@@H](CC(=O)NCc1cccc3c1COB3O)C(=N)N2C(C)=N] |
| 198 | 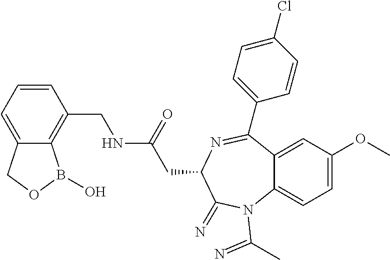 [SMILES: COc1ccc2c(c1)C(c1ccc(Cl)cc1)=N[C@@H](CC(=O)NCc1cccc3c1B(O)OC3)C(=N)N2C(C)=N] |
| 199 | 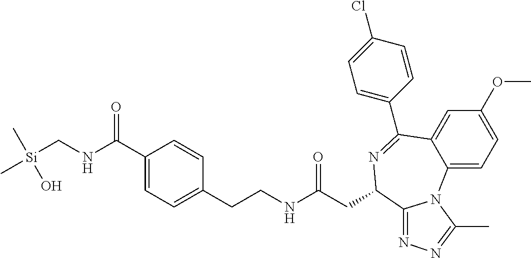 |
| 200 | 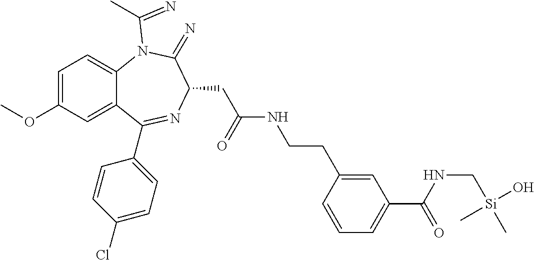 [SMILES: COc1ccc2c(c1)C(c1ccc(Cl)cc1)=N[C@@H](CC(=O)NCCc1cccc(C(=O)NC[Si](C)(C)O)c1)C(=N)N2C(C)=N] |
| 201 | 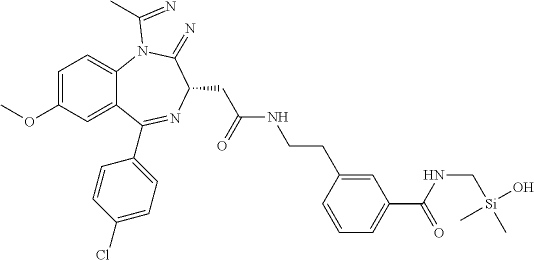 [SMILES: COc1ccc2c(c1)C(c1ccc(Cl)cc1)=N[C@@H](CC(=O)NCCc1cccc(C(=O)NC[Si](C)(C)O)c1)C(=N)N2C(C)=N] |
| 202 | 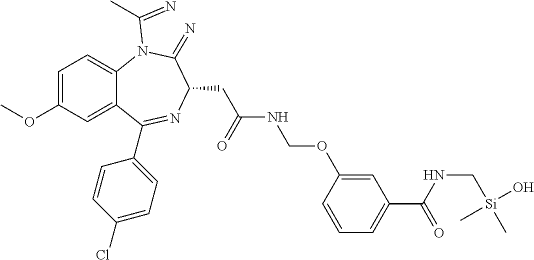 [SMILES: COc1ccc2c(c1)C(c1ccc(Cl)cc1)=N[C@@H](CC(=O)NCOc1cccc(C(=O)NC[Si](C)(C)O)c1)C(=N)N2C(C)=N] |
| 203 | 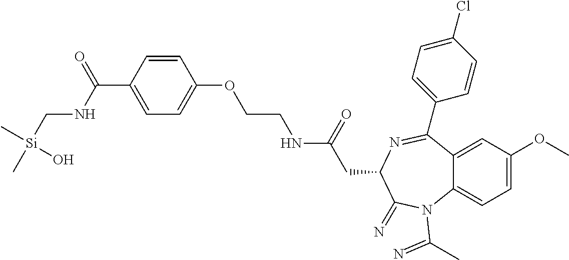 |
| 204 | 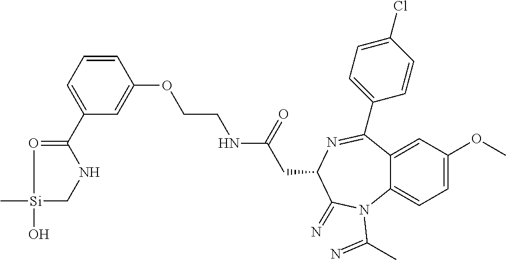 |
| 205 | 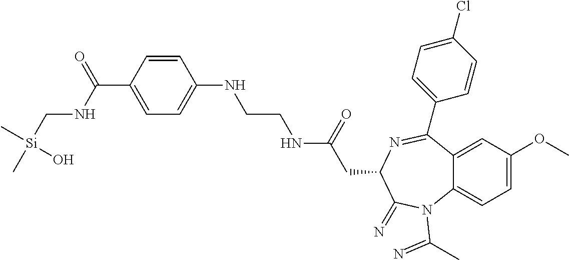 |
| 206 | 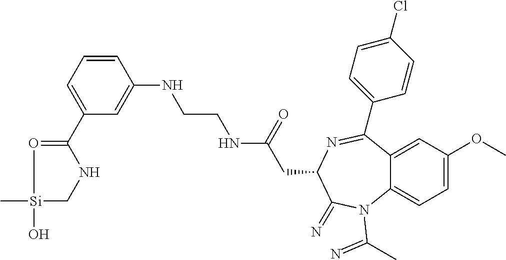 |
| 207 | 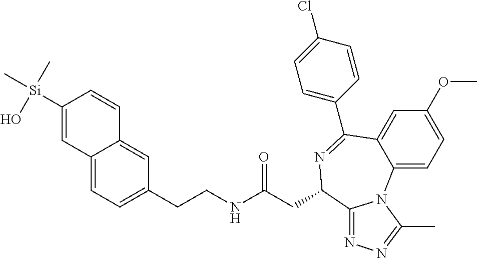 |
| 208 | 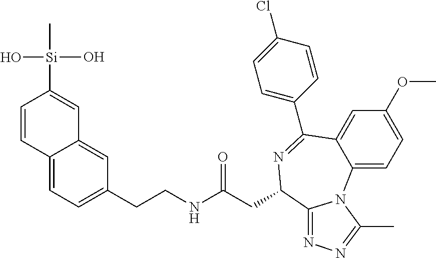 [SMILES: COc1ccc2c(c1)C(c1ccc(Cl)cc1)=N[C@@H](CC(=O)NCCc1ccc3ccc([Si](C)(O)O)cc3c1)c1nnc(C)n1-2] |
| 209 | 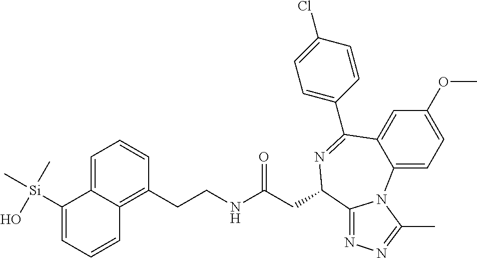 |
| 210 | 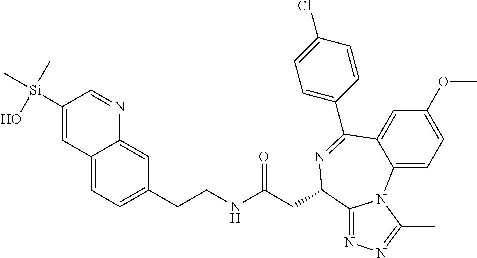 |
| 211 | 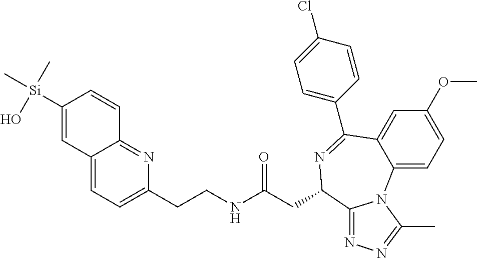 |
| 212 | 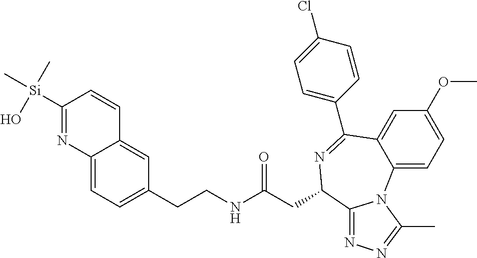 |
| 213 | 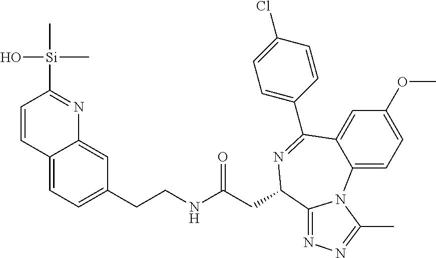 [SMILES: COc1ccc2c(c1)C(c1ccc(Cl)cc1)=N[C@@H](CC(=O)NCCc1ccc3ccc([Si](C)(C)O)nc3c1)c1nnc(C)n1-2] |
| 214 | 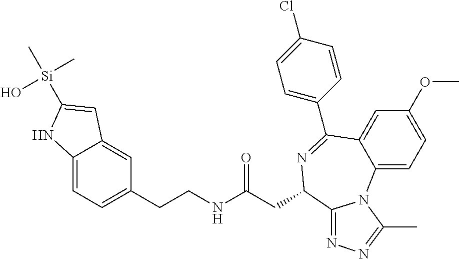 [SMILES: COc1ccc2c(c1)C(c1ccc(Cl)cc1)=N[C@@H](CC(=O)NCCc1ccc3[nH]c([Si](C)(C)O)cc3c1)c1nnc(C)n1-2] |
| 215 | 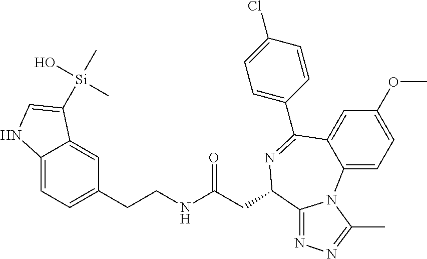 [SMILES: COc1ccc2c(c1)C(c1ccc(Cl)cc1)=N[C@@H](CC(=O)NCCc1ccc3[nH]cc([Si](C)(C)O)c3c1)c1nnc(C)n1-2] |
| 216 | 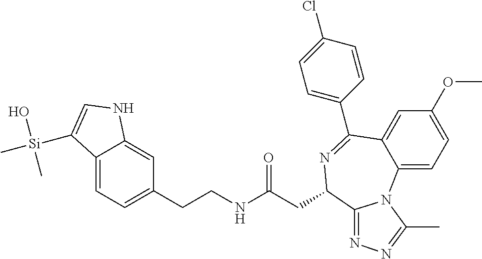 |
| 217 | 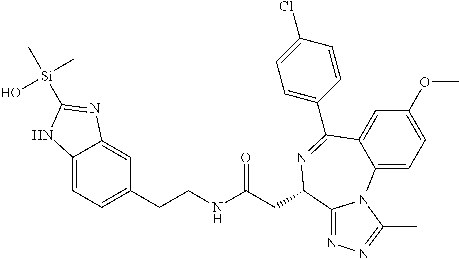 [SMILES: COc1ccc2c(c1)C(c1ccc(Cl)cc1)=N[C@@H](CC(=O)NCCc1ccc3[nH]c([Si](C)(C)O)nc3c1)c1nnc(C)n1-2] |
| 218 | 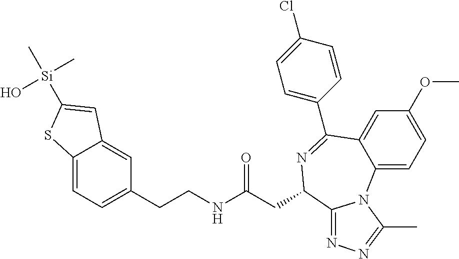 [SMILES: COc1ccc2c(c1)C(c1ccc(Cl)cc1)=N[C@@H](CC(=O)NCCc1ccc3sc([Si](C)(C)O)cc3c1)c1nnc(C)n1-2] |
| 219 | 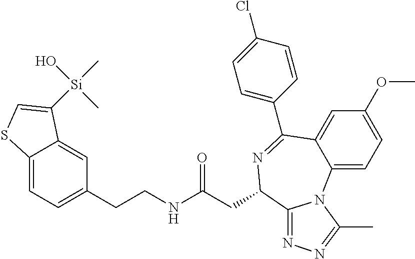 [SMILES: COc1ccc2c(c1)C(c1ccc(Cl)cc1)=N[C@@H](CC(=O)NCCc1ccc3scc([Si](C)(C)O)c3c1)c1nnc(C)n1-2] |
| 220 | 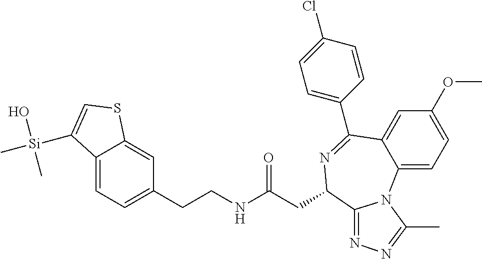 |
| 221 | 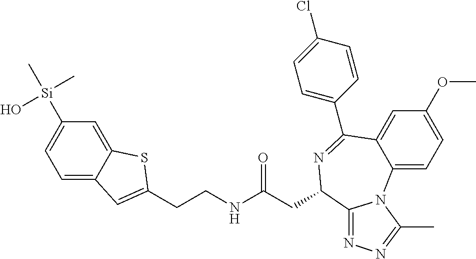 [SMILES: COc1ccc2c(c1)C(c1ccc(Cl)cc1)=N[C@@H](CC(=O)NCCc1cc3ccc([Si](C)(C)O)cc3s1)c1nnc(C)n1-2] |
| 222 | 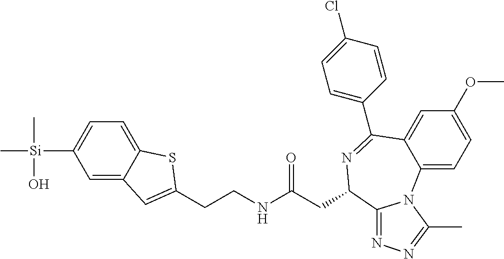 |
| 223 | 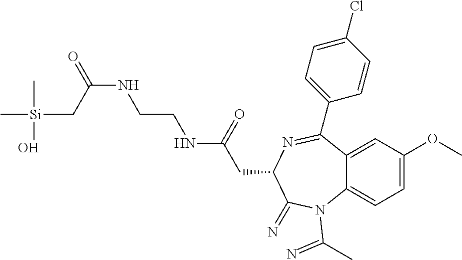 |
| 224 | 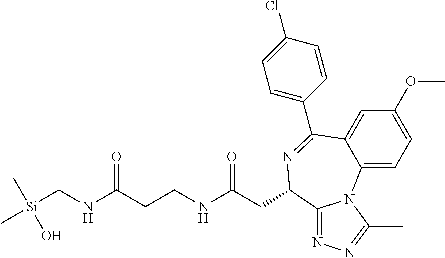 |
| 225 | 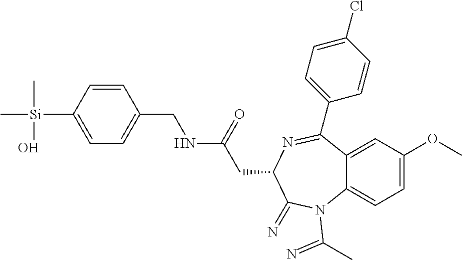 |
| 226 | 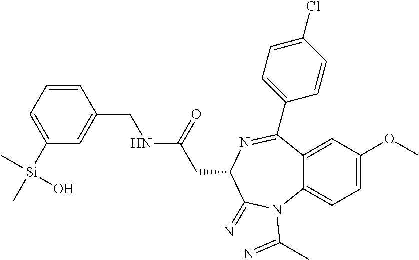 |
| 227 | 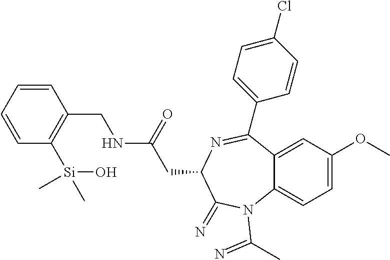 [SMILES: COc1ccc2c(c1)C(c1ccc(Cl)cc1)=N[C@@H](CC(=O)NCc1ccccc1[Si](C)(C)O)C(=N)N2C(C)=N] |
| 228 | 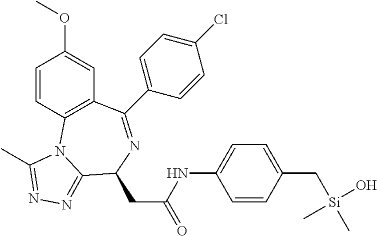 [SMILES: COc1ccc2c(c1)C(c1ccc(Cl)cc1)=N[C@@H](CC(=O)Nc1ccc(C[Si](C)(C)O)cc1)c1nnc(C)n1-2] |
| 229 | 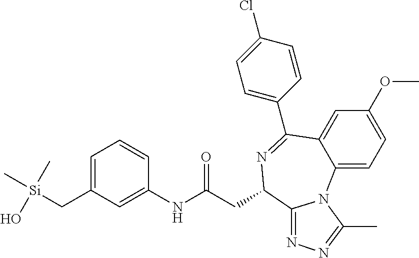 |
| 230 | 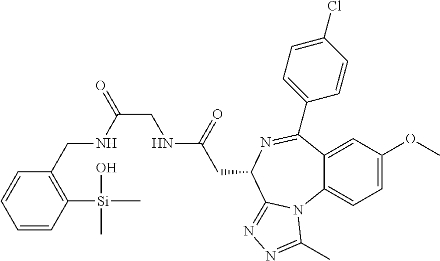 [SMILES: COc1ccc2c(c1)C(c1ccc(Cl)cc1)=N[C@@H](CC(=O)NCC(=O)NCc1ccccc1[Si](C)(C)O)c1nnc(C)n1-2] |
| 231 | 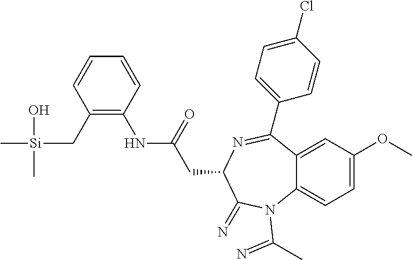 |
| 232 | 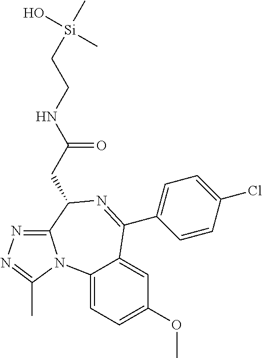 [SMILES: COc1ccc2c(c1)C(c1ccc(Cl)cc1)=N[C@@H](CC(=O)NCC[Si](C)(C)O)c1nnc(C)n1-2] |
| 233 | 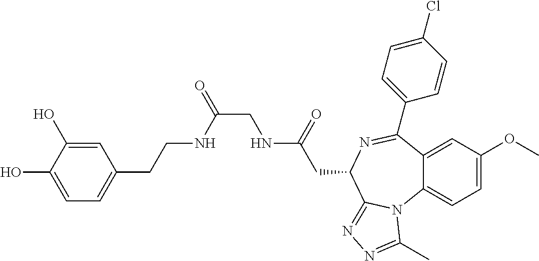 [SMILES: COc1ccc2c(c1)C(c1ccc(Cl)cc1)=N[C@@H](CC(=O)NCC(=O)NCCc1ccc(O)c(O)c1)c1nnc(C)n1-2] |
| 234 | 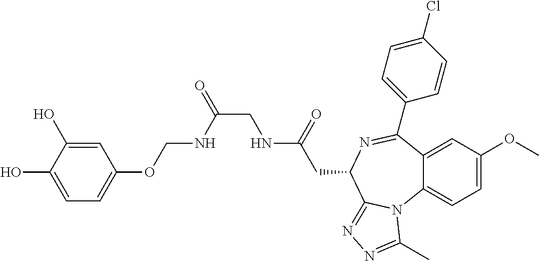 [SMILES: COc1ccc2c(c1)C(c1ccc(Cl)cc1)=N[C@@H](CC(=O)NCC(=O)NCOc1ccc(O)c(O)c1)c1nnc(C)n1-2] |
| 235 | 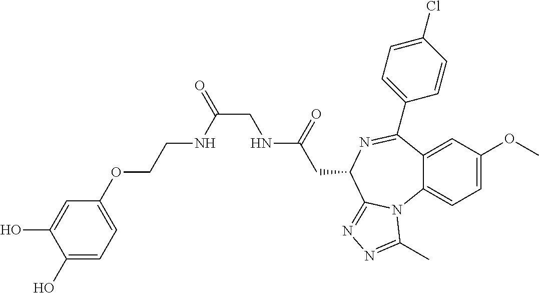 [SMILES: COc1ccc2c(c1)C(c1ccc(Cl)cc1)=N[C@@H](CC(=O)NCC(=O)NCCOc1ccc(O)c(O)c1)c1nnc(C)n1-2] |
| 236 | 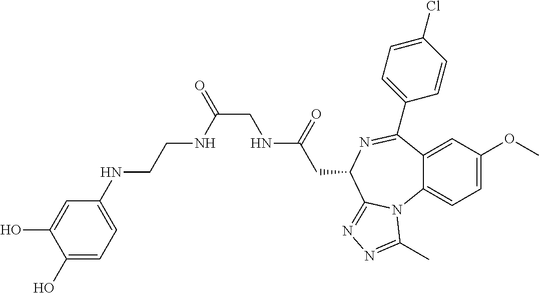 [SMILES: COc1ccc2c(c1)C(c1ccc(Cl)cc1)=N[C@@H](CC(=O)NCC(=O)NCCNc1ccc(O)c(O)c1)c1nnc(C)n1-2] |
| 237 | 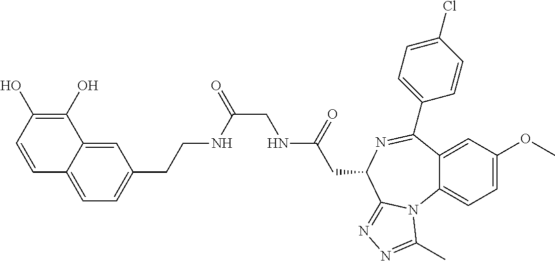 [SMILES: COc1ccc2c(c1)C(c1ccc(Cl)cc1)=N[C@@H](CC(=O)NCC(=O)NCCc1ccc3ccc(O)c(O)c3c1)c1nnc(C)n1-2] |
| 238 | 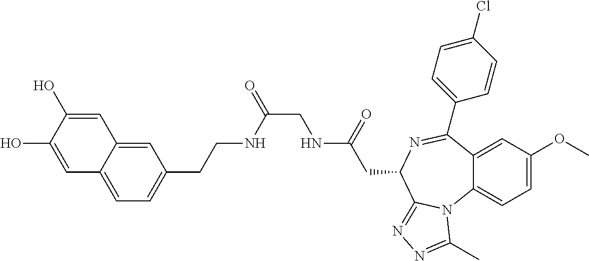 [SMILES: COc1ccc2c(c1)C(c1ccc(Cl)cc1)=N[C@@H](CC(=O)NCC(=O)NCCc1ccc3cc(O)c(O)cc3c1)c1nnc(C)n1-2] |
| 239 | 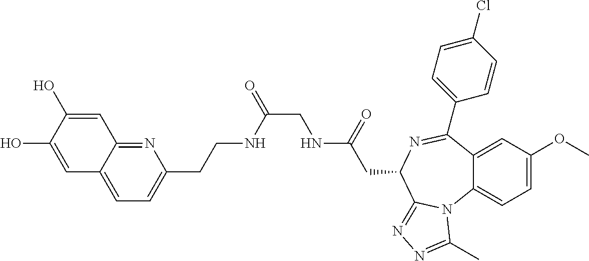 [SMILES: COc1ccc2c(c1)C(c1ccc(Cl)cc1)=N[C@@H](CC(=O)NCC(=O)NCCc1ccc3cc(O)c(O)cc3n1)c1nnc(C)n1-2] |
| 240 | 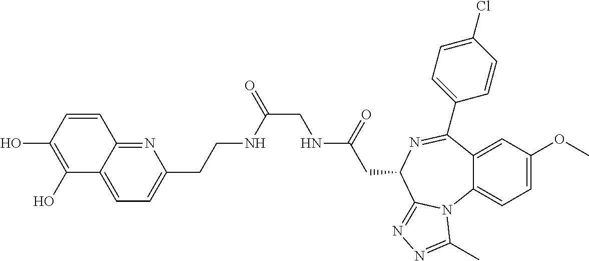 [SMILES: COc1ccc2c(c1)C(c1ccc(Cl)cc1)=N[C@@H](CC(=O)NCC(=O)NCCc1ccc3c(O)c(O)ccc3n1)c1nnc(C)n1-2] |
| 241 | 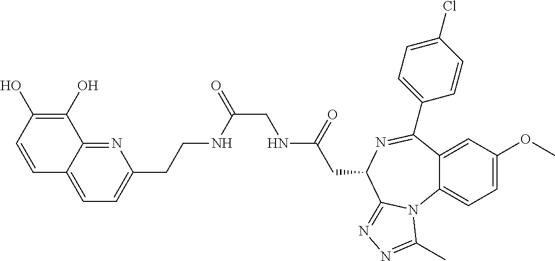 [SMILES: COc1ccc2c(c1)C(c1ccc(Cl)cc1)=N[C@@H](CC(=O)NCC(=O)NCCc1ccc3ccc(O)c(O)c3n1)c1nnc(C)n1-2] |
| 242 | 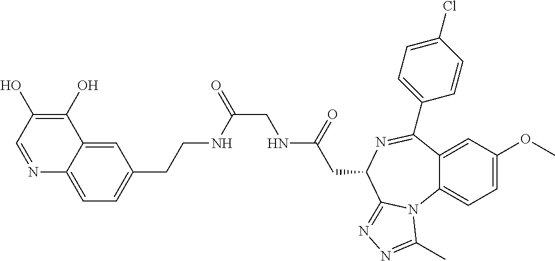 [SMILES: COc1ccc2c(c1)C(c1ccc(Cl)cc1)=N[C@@H](CC(=O)NCC(=O)NCCc1ccc3ncc(O)c(O)c3c1)c1nnc(C)n1-2] |
| 243 | 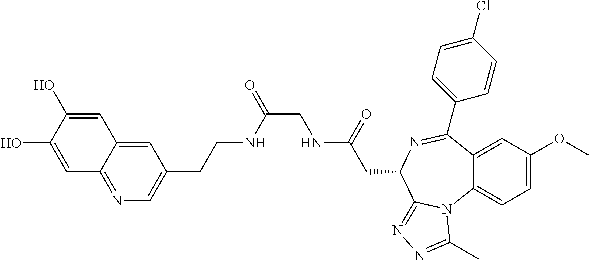 [SMILES: COc1ccc2c(c1)C(c1ccc(Cl)cc1)=N[C@@H](CC(=O)NCC(=O)NCCc1cnc3cc(O)c(O)cc3c1)c1nnc(C)n1-2] |
| 244 | 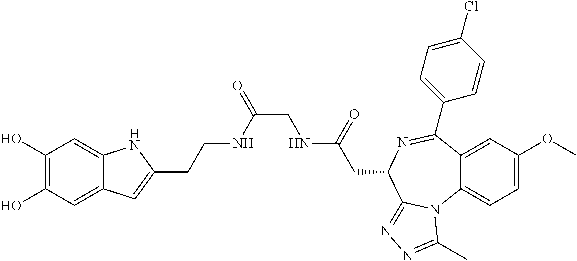 [SMILES: COc1ccc2c(c1)C(c1ccc(Cl)cc1)=N[C@@H](CC(=O)NCC(=O)NCCc1cc3cc(O)c(O)cc3[nH]1)c1nnc(C)n1-2] |
| 245 | 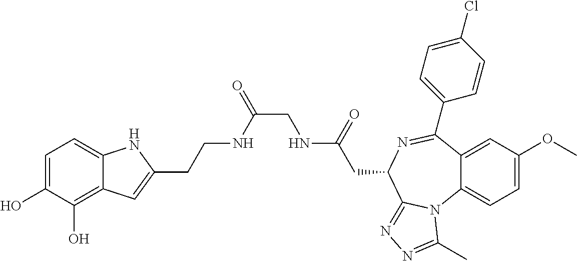 [SMILES: COc1ccc2c(c1)C(c1ccc(Cl)cc1)=N[C@@H](CC(=O)NCC(=O)NCCc1cc3c(O)c(O)ccc3[nH]1)c1nnc(C)n1-2] |
| 246 | 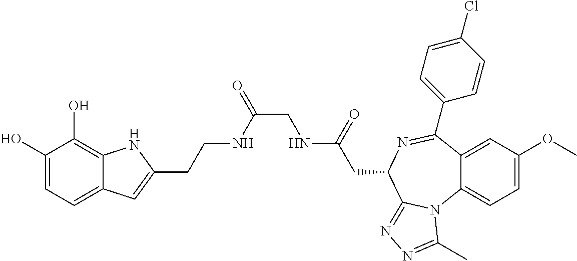 [SMILES: COc1ccc2c(c1)C(c1ccc(Cl)cc1)=N[C@@H](CC(=O)NCC(=O)NCCc1cc3ccc(O)c(O)c3[nH]1)c1nnc(C)n1-2] |
| 247 | 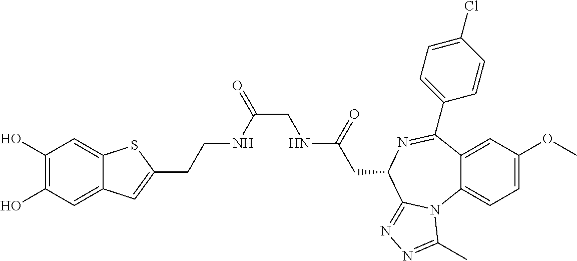 [SMILES: COc1ccc2c(c1)C(c1ccc(Cl)cc1)=N[C@@H](CC(=O)NCC(=O)NCCc1cc3cc(O)c(O)cc3s1)c1nnc(C)n1-2] |
| 248 | 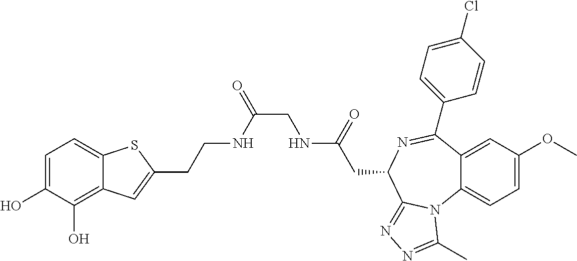 [SMILES: COc1ccc2c(c1)C(c1ccc(Cl)cc1)=N[C@@H](CC(=O)NCC(=O)NCCc1cc3c(O)c(O)ccc3s1)c1nnc(C)n1-2] |
| 249 | 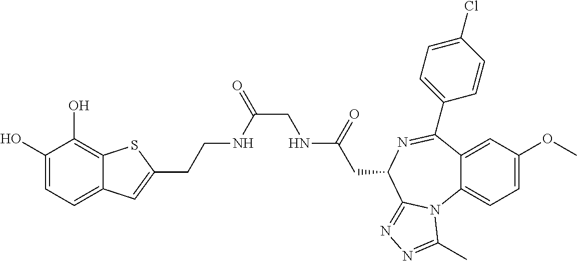 [SMILES: COc1ccc2c(c1)C(c1ccc(Cl)cc1)=N[C@@H](CC(=O)NCC(=O)NCCc1cc3ccc(O)c(O)c3s1)c1nnc(C)n1-2] |
| 250 | 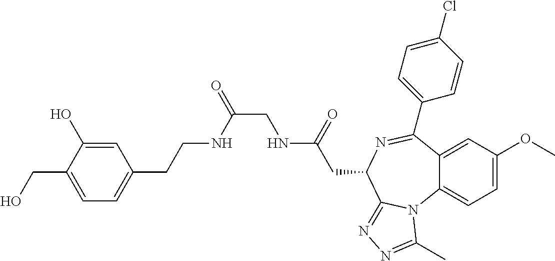 [SMILES: COc1ccc2c(c1)C(c1ccc(Cl)cc1)=N[C@@H](CC(=O)NCC(=O)NCCc1ccc(CO)c(O)c1)c1nnc(C)n1-2] |
| 251 | 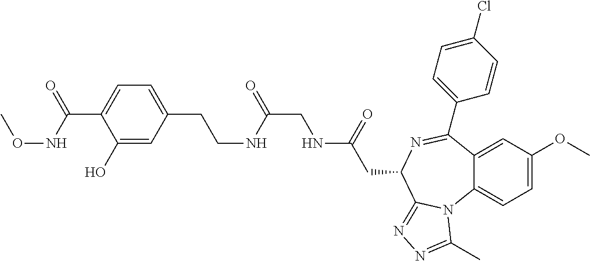 |
| 252 | 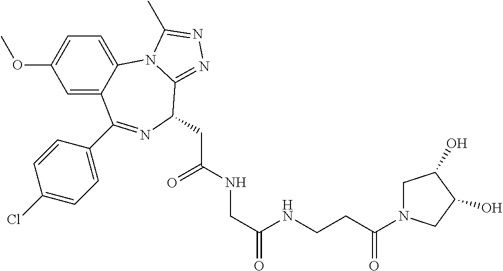 [SMILES: COc1ccc2c(c1)C(c1ccc(Cl)cc1)=N[C@@H](CC(=O)NCC(=O)NCCC(=O)N1C[C@@H](O)[C@@H](O)C1)c1nnc(C)n1-2] |
| 253 | 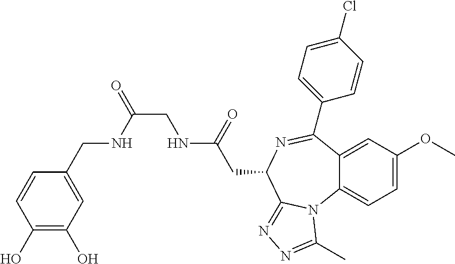 [SMILES: COc1ccc2c(c1)C(c1ccc(Cl)cc1)=N[C@@H](CC(=O)NCC(=O)NCc1ccc(O)c(O)c1)c1nnc(C)n1-2] |
| 254 | 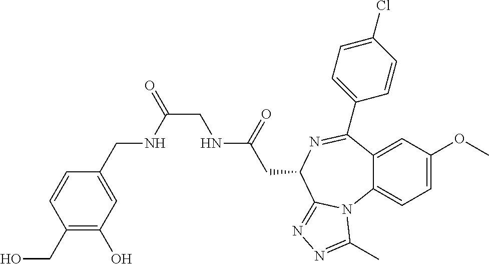 [SMILES: COc1ccc2c(c1)C(c1ccc(Cl)cc1)=N[C@@H](CC(=O)NCC(=O)NCc1ccc(CO)c(O)c1)c1nnc(C)n1-2] |
| 255 | 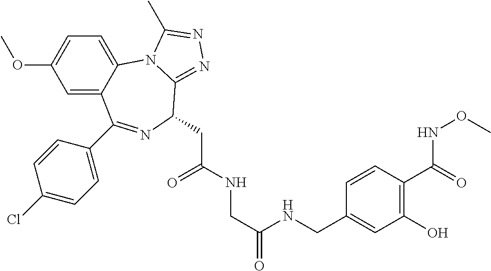 |
| 256 | 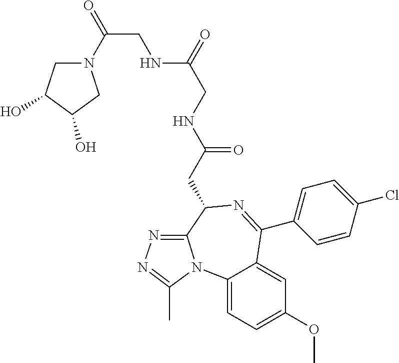 [SMILES: COc1ccc2c(c1)C(c1ccc(Cl)cc1)=N[C@@H](CC(=O)NCC(=O)NCC(=O)N1C[C@@H](O)[C@@H](O)C1)c1nnc(C)n1-2] |
| 257 | 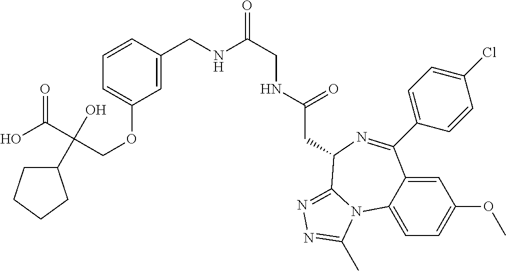 [SMILES: COc1ccc2c(c1)C(c1ccc(Cl)cc1)=N[C@@H](CC(=O)NCC(=O)NCc1cccc(OCC(O)(C(=O)O)C3CCCC3)c1)c1nnc(C)n1-2] |
| 258 | 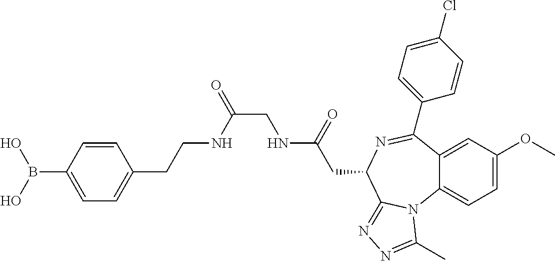 [SMILES: COc1ccc2c(c1)C(c1ccc(Cl)cc1)=N[C@@H](CC(=O)NCC(=O)NCCc1ccc(B(O)O)cc1)c1nnc(C)n1-2] |
| 259 | 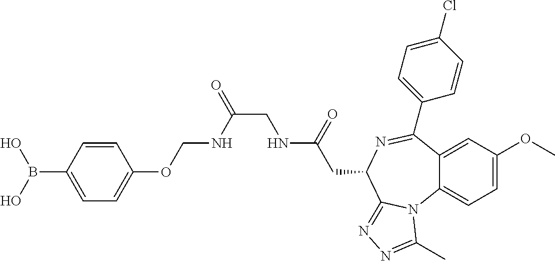 [SMILES: COc1ccc2c(c1)C(c1ccc(Cl)cc1)=N[C@@H](CC(=O)NCC(=O)NCOc1ccc(B(O)O)cc1)c1nnc(C)n1-2] |
| 260 | 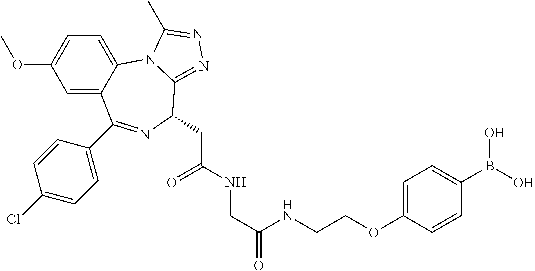 [SMILES: COc1ccc2c(c1)C(c1ccc(Cl)cc1)=N[C@@H](CC(=O)NCC(=O)NCCOc1ccc(B(O)O)cc1)c1nnc(C)n1-2] |
| 261 | 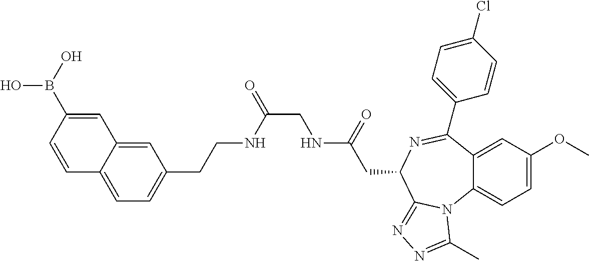 [SMILES: COc1ccc2c(c1)C(c1ccc(Cl)cc1)=N[C@@H](CC(=O)NCC(=O)NCCc1ccc3ccc(B(O)O)cc3c1)c1nnc(C)n1-2] |
| 262 | 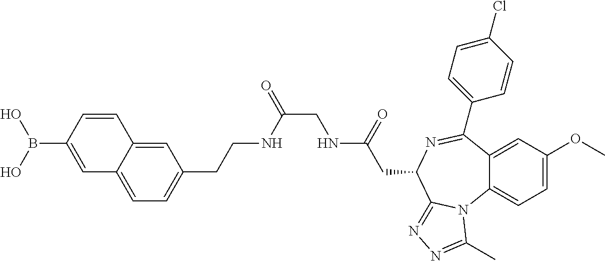 [SMILES: COc1ccc2c(c1)C(c1ccc(Cl)cc1)=N[C@@H](CC(=O)NCC(=O)NCCc1ccc3cc(B(O)O)ccc3c1)c1nnc(C)n1-2] |
| 263 | 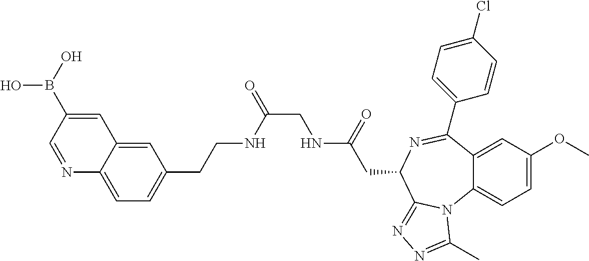 [SMILES: COc1ccc2c(c1)C(c1ccc(Cl)cc1)=N[C@@H](CC(=O)NCC(=O)NCCc1ccc3ncc(B(O)O)cc3c1)c1nnc(C)n1-2] |
| 264 | 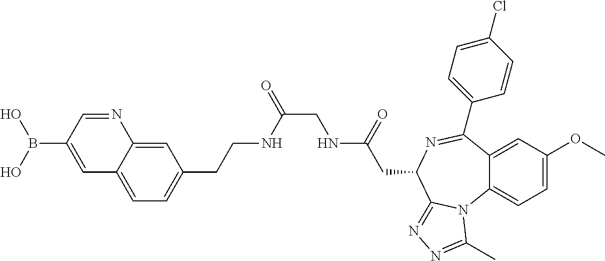 [SMILES: COc1ccc2c(c1)C(c1ccc(Cl)cc1)=N[C@@H](CC(=O)NCC(=O)NCCc1ccc3cc(B(O)O)cnc3c1)c1nnc(C)n1-2] |
| 265 | 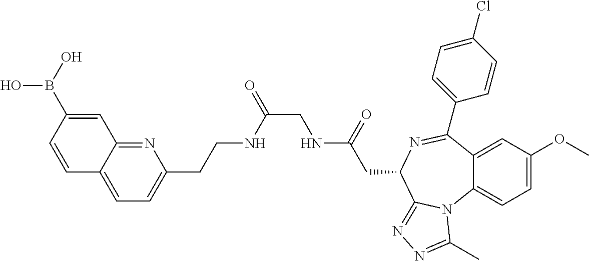 [SMILES: COc1ccc2c(c1)C(c1ccc(Cl)cc1)=N[C@@H](CC(=O)NCC(=O)NCCc1ccc3ccc(B(O)O)cc3n1)c1nnc(C)n1-2] |
| 266 | 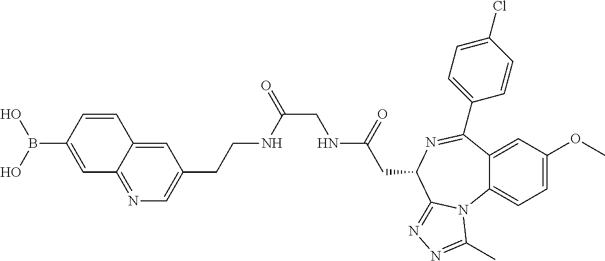 [SMILES: COc1ccc2c(c1)C(c1ccc(Cl)cc1)=N[C@@H](CC(=O)NCC(=O)NCCc1cnc3cc(B(O)O)ccc3c1)c1nnc(C)n1-2] |
| 267 | 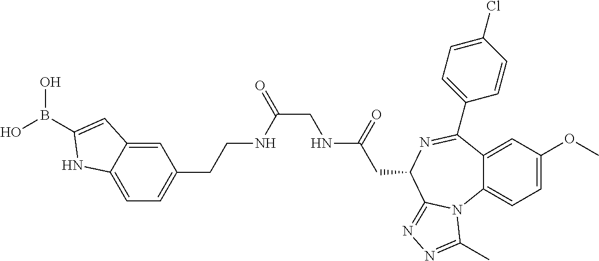 [SMILES: COc1ccc2c(c1)C(c1ccc(Cl)cc1)=N[C@@H](CC(=O)NCC(=O)NCCc1ccc3[nH]c(B(O)O)cc3c1)c1nnc(C)n1-2] |
| 268 | 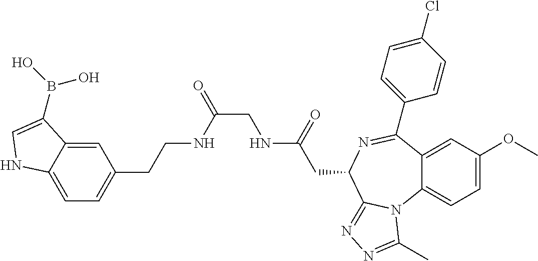 [SMILES: COc1ccc2c(c1)C(c1ccc(Cl)cc1)=N[C@@H](CC(=O)NCC(=O)NCCc1ccc3[nH]cc(B(O)O)c3c1)c1nnc(C)n1-2] |
| 269 | 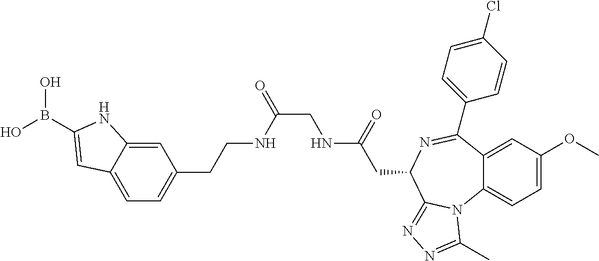 [SMILES: COc1ccc2c(c1)C(c1ccc(Cl)cc1)=N[C@@H](CC(=O)NCC(=O)NCCc1ccc3cc(B(O)O)[nH]c3c1)c1nnc(C)n1-2] |
| 270 | 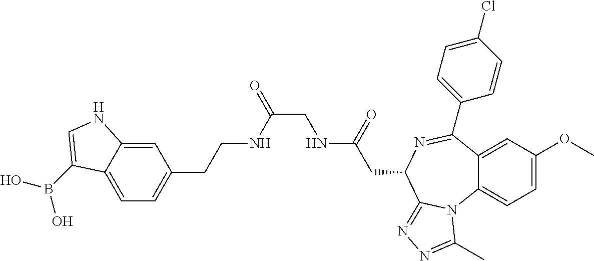 [SMILES: COc1ccc2c(c1)C(c1ccc(Cl)cc1)=N[C@@H](CC(=O)NCC(=O)NCCc1ccc3c(B(O)O)c[nH]c3c1)c1nnc(C)n1-2] |
| 271 | 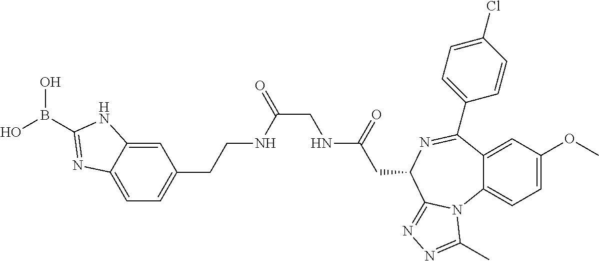 [SMILES: COc1ccc2c(c1)C(c1ccc(Cl)cc1)=N[C@@H](CC(=O)NCC(=O)NCCc1ccc3nc(B(O)O)[nH]c3c1)c1nnc(C)n1-2] |
| 272 | 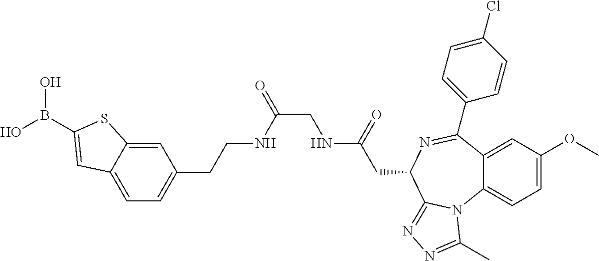 [SMILES: COc1ccc2c(c1)C(c1ccc(Cl)cc1)=N[C@@H](CC(=O)NCC(=O)NCCc1ccc3cc(B(O)O)sc3c1)c1nnc(C)n1-2] |
| 273 | 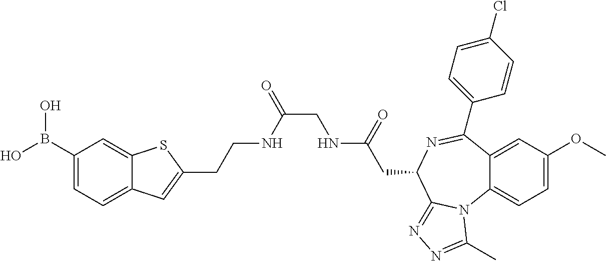 [SMILES: COc1ccc2c(c1)C(c1ccc(Cl)cc1)=N[C@@H](CC(=O)NCC(=O)NCCc1cc3ccc(B(O)O)cc3s1)c1nnc(C)n1-2] |
| 274 | 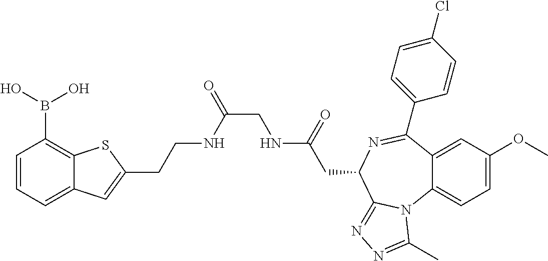 [SMILES: COc1ccc2c(c1)C(c1ccc(Cl)cc1)=N[C@@H](CC(=O)NCC(=O)NCCc1cc3cccc(B(O)O)c3s1)c1nnc(C)n1-2] |
| 275 | 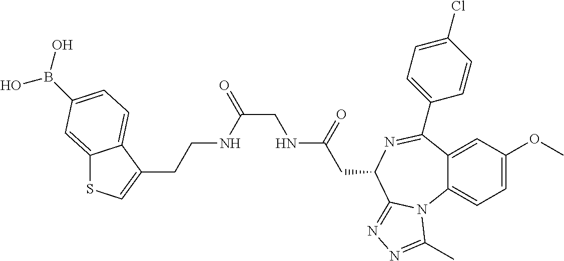 [SMILES: COc1ccc2c(c1)C(c1ccc(Cl)cc1)=N[C@@H](CC(=O)NCC(=O)NCCc1csc3cc(B(O)O)ccc13)c1nnc(C)n1-2] |
| 276 | 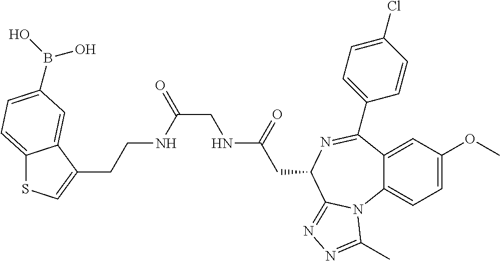 [SMILES: COc1ccc2c(c1)C(c1ccc(Cl)cc1)=N[C@@H](CC(=O)NCC(=O)NCCc1csc3ccc(B(O)O)cc13)c1nnc(C)n1-2] |
| 277 | 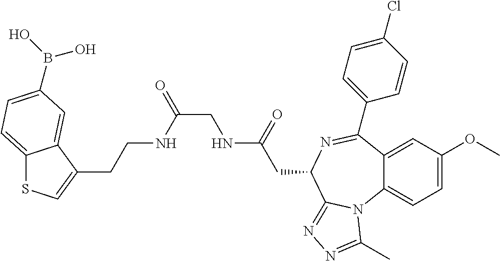 [SMILES: COc1ccc2c(c1)C(c1ccc(Cl)cc1)=N[C@@H](CC(=O)NCC(=O)NCCc1csc3ccc(B(O)O)cc13)c1nnc(C)n1-2] |
| 278 | 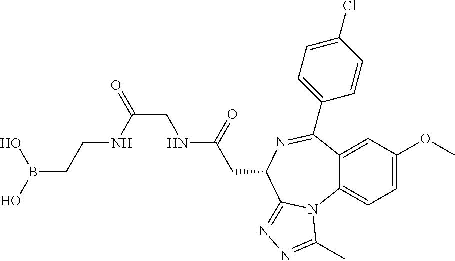 [SMILES: COc1ccc2c(c1)C(c1ccc(Cl)cc1)=N[C@@H](CC(=O)NCC(=O)NCCB(O)O)c1nnc(C)n1-2] |
| 279 | 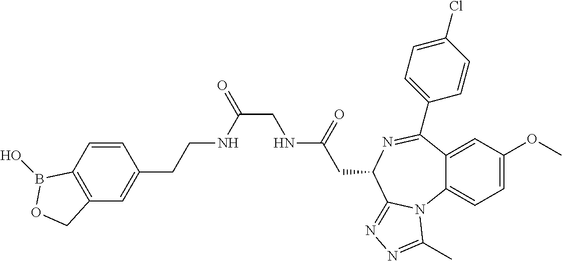 [SMILES: COc1ccc2c(c1)C(c1ccc(Cl)cc1)=N[C@@H](CC(=O)NCC(=O)NCCc1ccc3c(c1)COB3O)c1nnc(C)n1-2] |
| 280 | 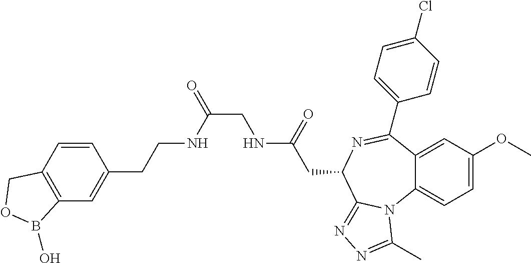 [SMILES: COc1ccc2c(c1)C(c1ccc(Cl)cc1)=N[C@@H](CC(=O)NCC(=O)NCCc1ccc3c(c1)B(O)OC3)c1nnc(C)n1-2] |
| 281 | 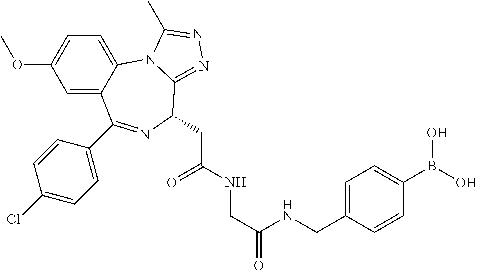 [SMILES: COc1ccc2c(c1)C(c1ccc(Cl)cc1)=N[C@@H](CC(=O)NCC(=O)NCc1ccc(B(O)O)cc1)c1nnc(C)n1-2] |
| 282 | 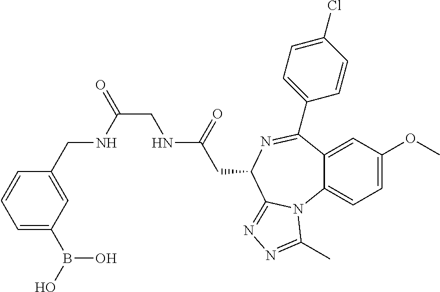 [SMILES: COc1ccc2c(c1)C(c1ccc(Cl)cc1)=N[C@@H](CC(=O)NCC(=O)NCc1cccc(B(O)O)c1)c1nnc(C)n1-2] |
| 283 | 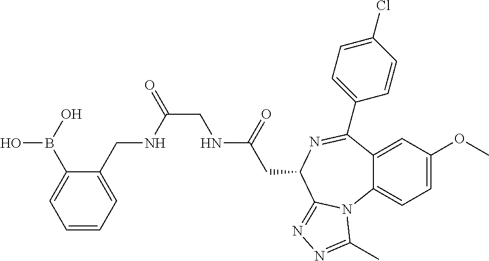 [SMILES: COc1ccc2c(c1)C(c1ccc(Cl)cc1)=N[C@@H](CC(=O)NCC(=O)NCc1ccccc1B(O)O)c1nnc(C)n1-2] |
| 284 | 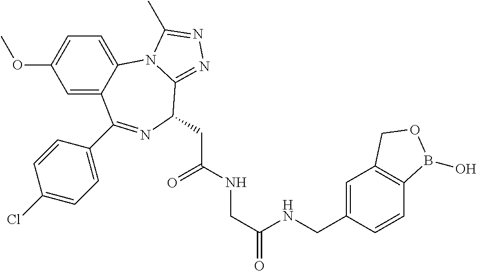 [SMILES: COc1ccc2c(c1)C(c1ccc(Cl)cc1)=N[C@@H](CC(=O)NCC(=O)NCc1ccc3c(c1)COB3O)c1nnc(C)n1-2] |
| 285 | 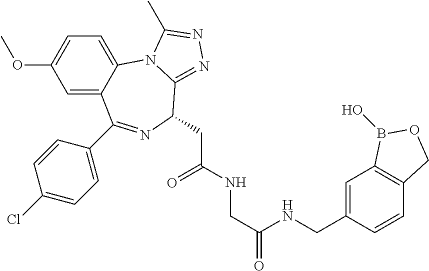 [SMILES: COc1ccc2c(c1)C(c1ccc(Cl)cc1)=N[C@@H](CC(=O)NCC(=O)NCc1ccc3c(c1)B(O)OC3)c1nnc(C)n1-2] |
| 286 | 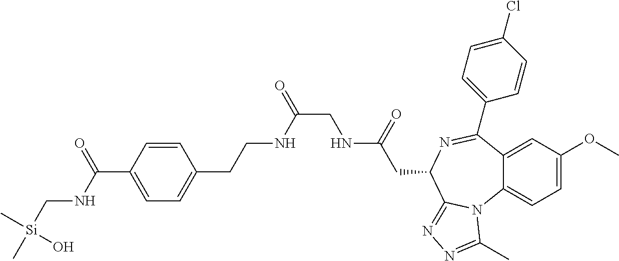 |
| 287 | 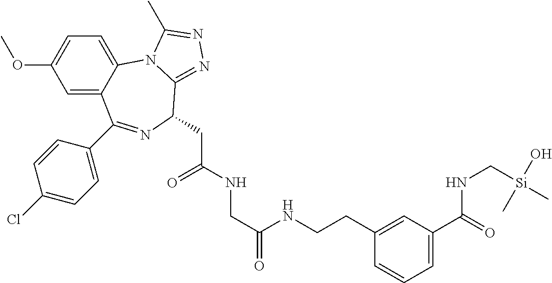 |
| 288 | 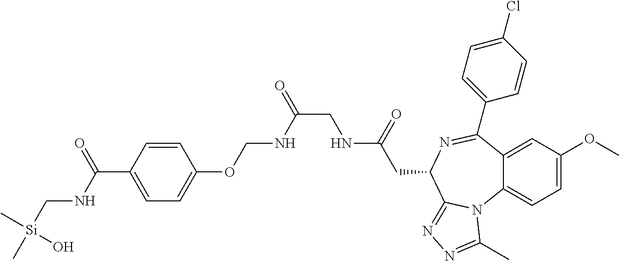 |
| 289 | 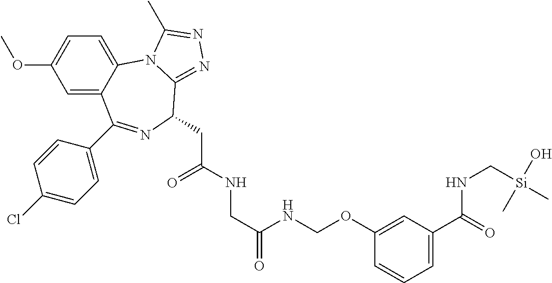 |
| 290 | 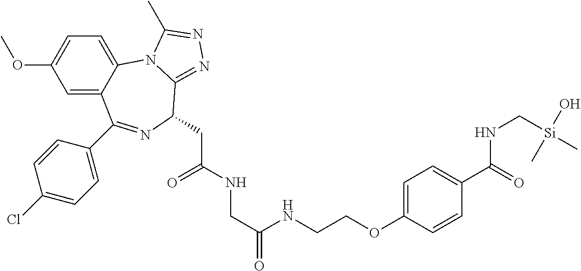 |
| 291 | 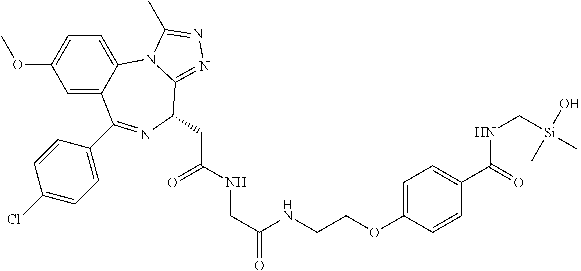 |
| 292 | 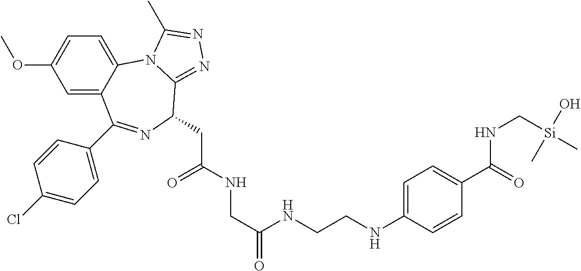 |
| 293 | 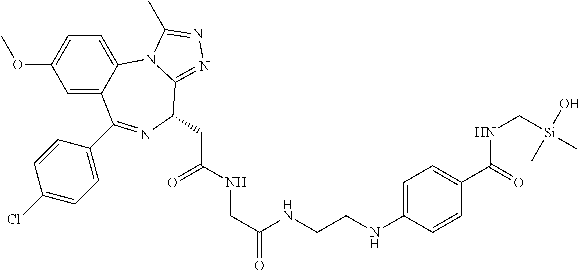 |
| 294 | 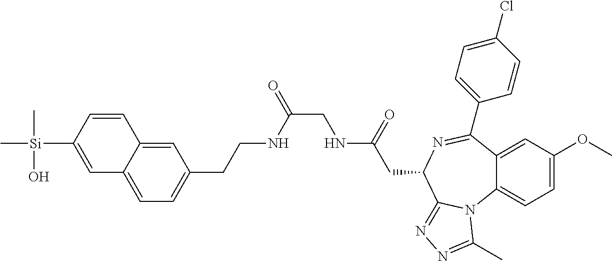 |
| 295 | 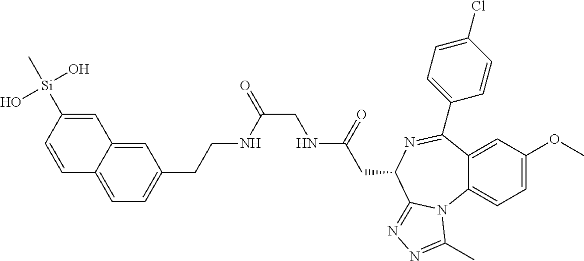 [SMILES: COc1ccc2c(c1)C(c1ccc(Cl)cc1)=N[C@@H](CC(=O)NCC(=O)NCCc1ccc3ccc([Si](C)(O)O)cc3c1)c1nnc(C)n1-2] |
| 296 | 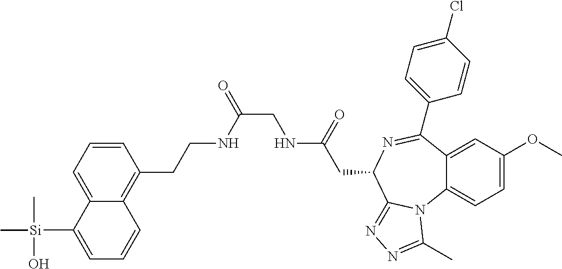 |
| 297 | 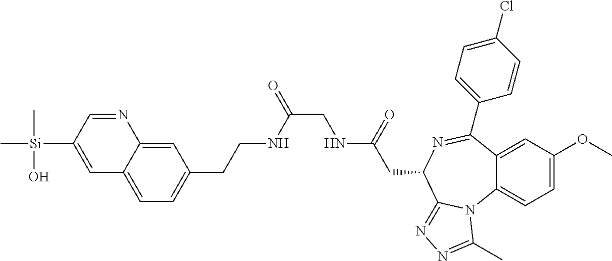 |
| 298 | 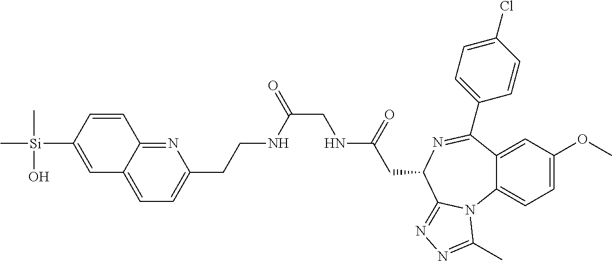 |
| 299 | 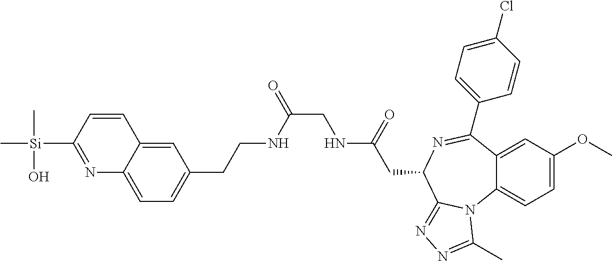 |
| 300 | 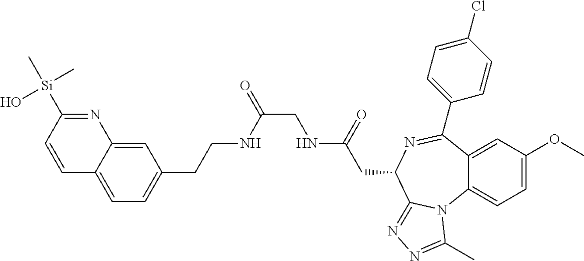 [SMILES: COc1ccc2c(c1)C(c1ccc(Cl)cc1)=N[C@@H](CC(=O)NCC(=O)NCCc1ccc3ccc([Si](C)(C)O)nc3c1)c1nnc(C)n1-2] |
| 301 | 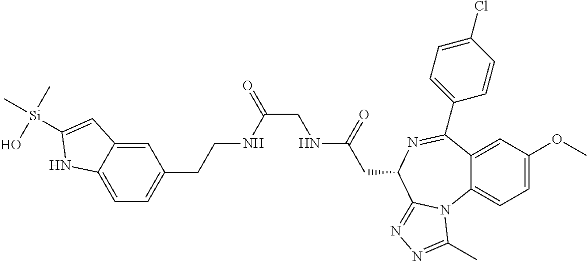 |
| 302 | 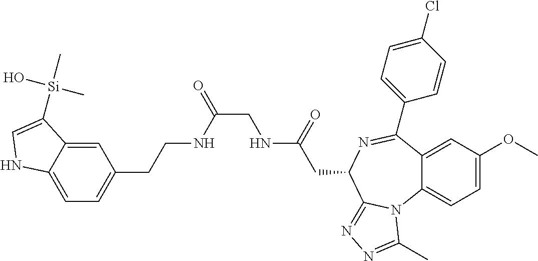 [SMILES: COc1ccc2c(c1)C(c1ccc(Cl)cc1)=N[C@@H](CC(=O)NCC(=O)NCCc1ccc3[nH]cc([Si](C)(C)O)c3c1)c1nnc(C)n1-2] |
| 303 | 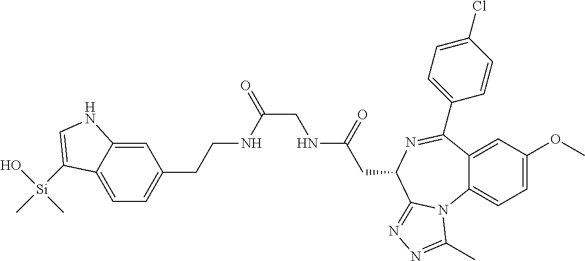 |
| 304 | 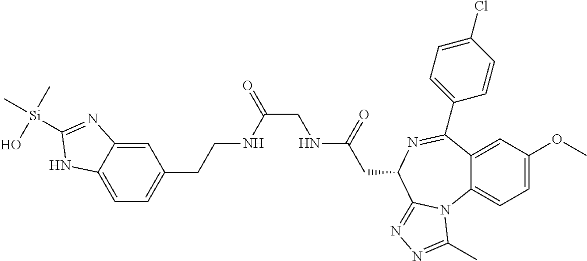 |
| 305 | 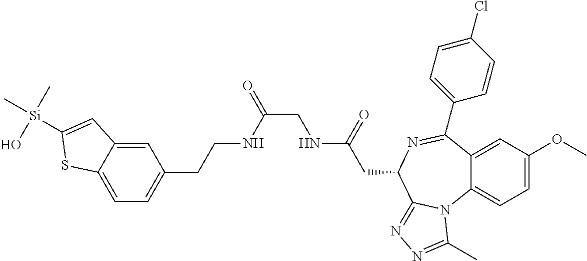 |
| 306 | 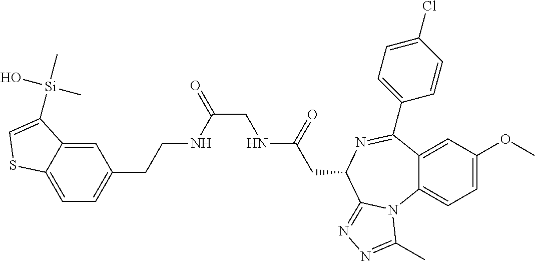 [SMILES: COc1ccc2c(c1)C(c1ccc(Cl)cc1)=N[C@@H](CC(=O)NCC(=O)NCCc1ccc3scc([Si](C)(C)O)c3c1)c1nnc(C)n1-2] |
| 307 | 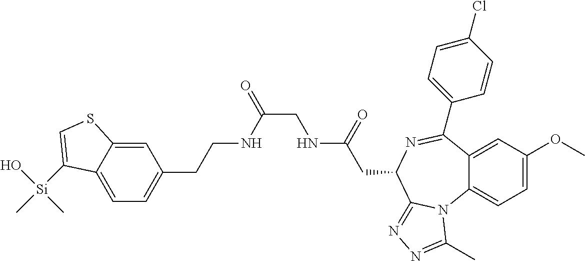 |
| 308 | 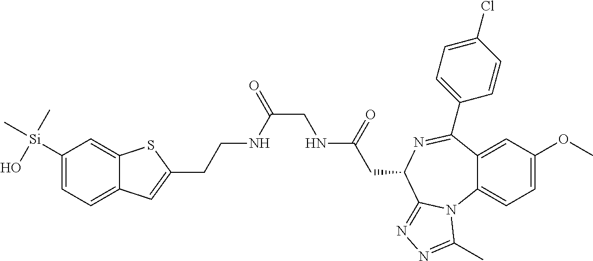 |
| 309 | 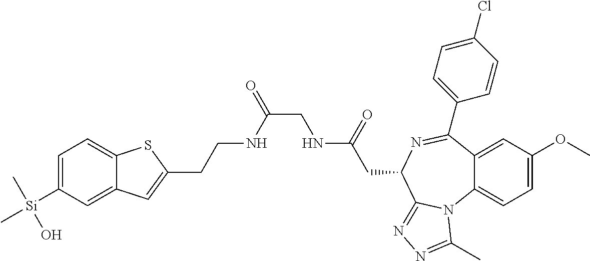 |
| 310 | 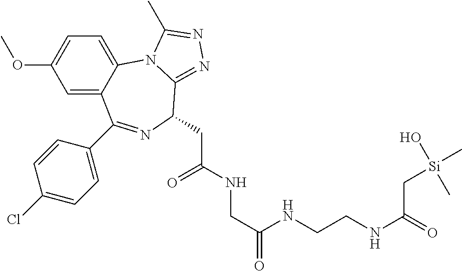 |
| 311 | 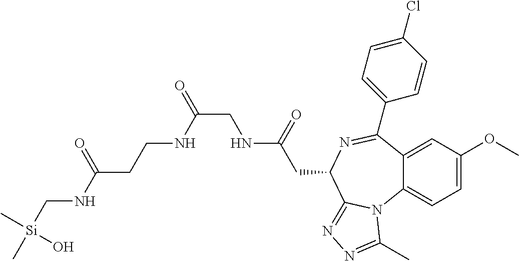 |
| 312 | 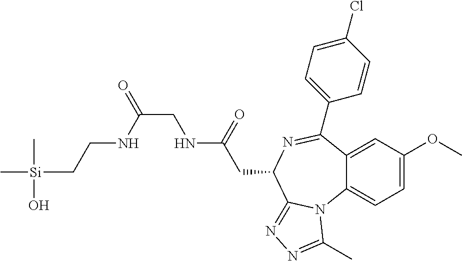 |
| 313 | 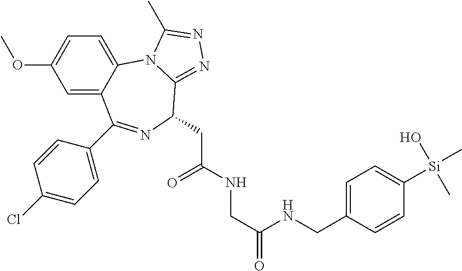 |
| 314 | 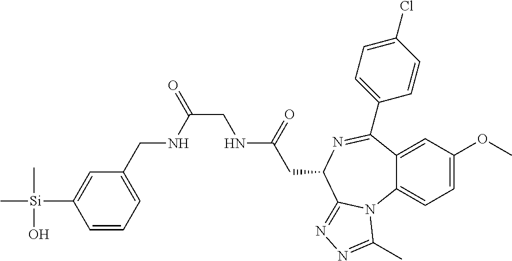 |

### Examples 315-351

Table Y contains monomers which may form a dimer with a second monomer. The monomer examples in Table Y, having boronic acid, diol or silanol linkers (Z) correspond to the structures in Table E.

TABLE Y

| | Compound Structure |
|---|---|
| No. | (e.g., X-Y-Z) |
| 315 | 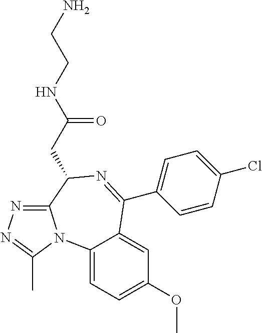 [SMILES: COc1ccc2c(c1)C(c1ccc(Cl)cc1)=N[C@@H](CC(=O)NCCN)c1nnc(C)n1-2] |
| 316 | 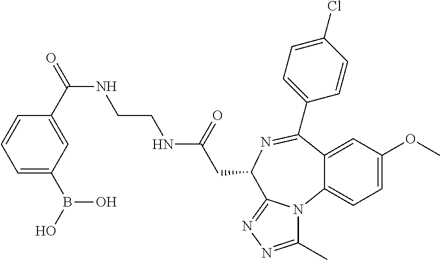 [SMILES: COc1ccc2c(c1)C(c1ccc(Cl)cc1)=N[C@@H](CC(=O)NCCNC(=O)c1cccc(B(O)O)c1)c1nnc(C)n1-2] |
| 317 | 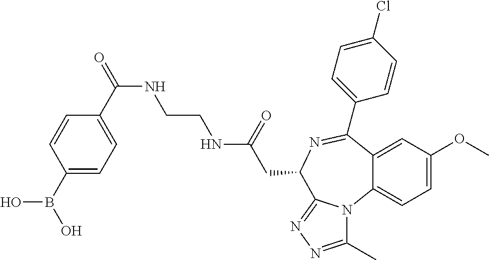 [SMILES: COc1ccc2c(c1)C(c1ccc(Cl)cc1)=N[C@@H](CC(=O)NCCNC(=O)c1ccc(B(O)O)cc1)c1nnc(C)n1-2] |
| 318 | 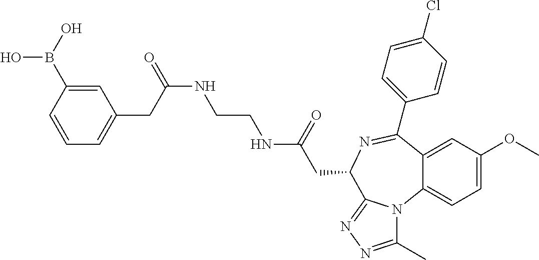 [SMILES: COc1ccc2c(c1)C(c1ccc(Cl)cc1)=N[C@@H](CC(=O)NCCNC(=O)Cc1cccc(B(O)O)c1)c1nnc(C)n1-2] |
| 319 | 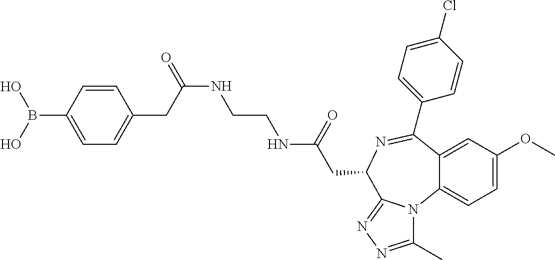 [SMILES: COc1ccc2c(c1)C(c1ccc(Cl)cc1)=N[C@@H](CC(=O)NCCNC(=O)Cc1ccc(B(O)O)cc1)c1nnc(C)n1-2] |
| 320 | 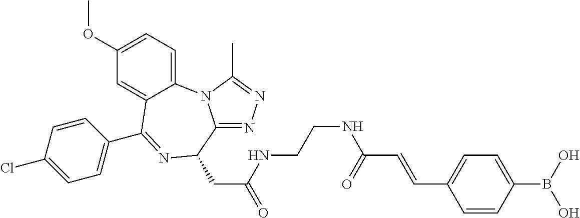 [SMILES: COc1ccc2c(c1)C(c1ccc(Cl)cc1)=N[C@@H](CC(=O)NCCNC(=O)/C=C/c1ccc(B(O)O)cc1)c1nnc(C)n1-2] |
| 321 | 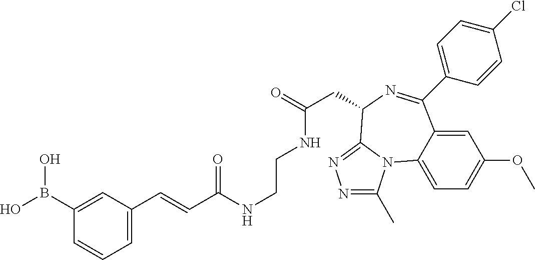 [SMILES: COc1ccc2c(c1)C(c1ccc(Cl)cc1)=N[C@@H](CC(=O)NCCNC(=O)/C=C/c1cccc(B(O)O)c1)c1nnc(C)n1-2] |
| 322 | 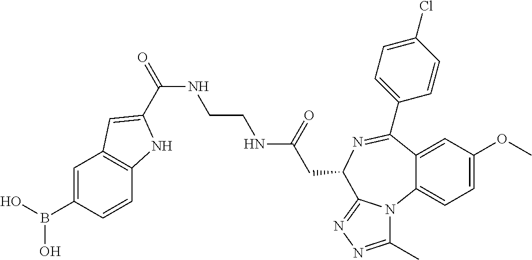 [SMILES: COc1ccc2c(c1)C(c1ccc(Cl)cc1)=N[C@@H](CC(=O)NCCNC(=O)c1cc3cc(B(O)O)ccc3[nH]1)c1nnc(C)n1-2] |
| 323 | 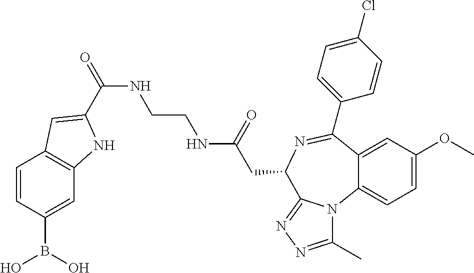 [SMILES: COc1ccc2c(c1)C(c1ccc(Cl)cc1)=N[C@@H](CC(=O)NCCNC(=O)c1cc3ccc(B(O)O)cc3[nH]1)c1nnc(C)n1-2] |
| 324 | 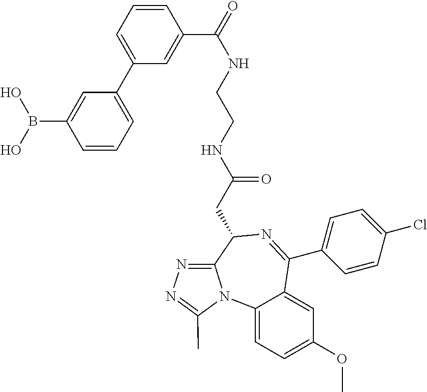 [SMILES: COc1ccc2c(c1)C(c1ccc(Cl)cc1)=N[C@@H](CC(=O)NCCNC(=O)c1cccc(-c3cccc(B(O)O)c3)c1)c1nnc(C)n1-2] |
| 325 | 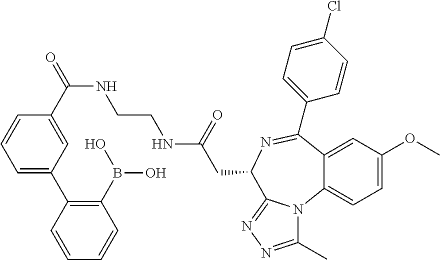 [SMILES: COc1ccc2c(c1)C(c1ccc(Cl)cc1)=N[C@@H](CC(=O)NCCNC(=O)c1cccc(-c3ccccc3B(O)O)c1)c1nnc(C)n1-2] |
| 326 | 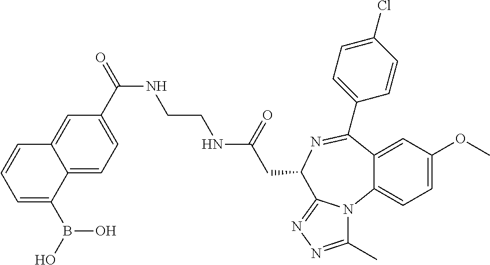 [SMILES: COc1ccc2c(c1)C(c1ccc(Cl)cc1)=N[C@@H](CC(=O)NCCNC(=O)c1ccc3c(B(O)O)cccc3c1)c1nnc(C)n1-2] |
| 327 | 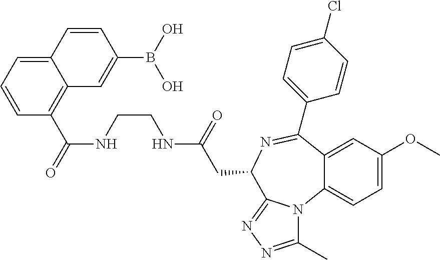 [SMILES: COc1ccc2c(c1)C(c1ccc(Cl)cc1)=N[C@@H](CC(=O)NCCNC(=O)c1cccc3ccc(B(O)O)cc13)c1nnc(C)n1-2] |
| 328 | 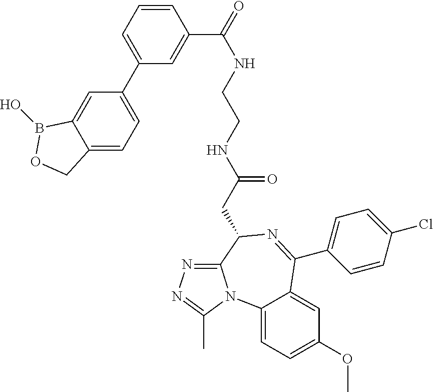 [SMILES: COc1ccc2c(c1)C(c1ccc(Cl)cc1)=N[C@@H](CC(=O)NCCNC(=O)c1cccc(-c3ccc4c(c3)B(O)OC4)c1)c1nnc(C)n1-2] |
| 329 | 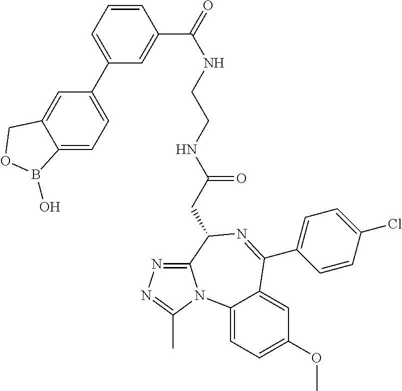 [SMILES: COc1ccc2c(c1)C(c1ccc(Cl)cc1)=N[C@@H](CC(=O)NCCNC(=O)c1cccc(-c3ccc4c(c3)COB4O)c1)c1nnc(C)n1-2] |
| 330 | 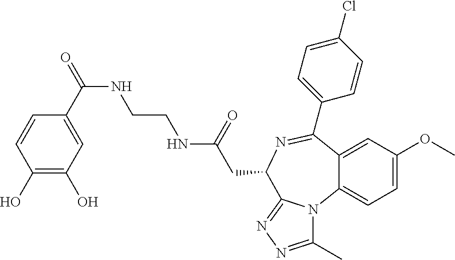 [SMILES: COc1ccc2c(c1)C(c1ccc(Cl)cc1)=N[C@@H](CC(=O)NCCNC(=O)c1ccc(O)c(O)c1)c1nnc(C)n1-2] |
| 331 | 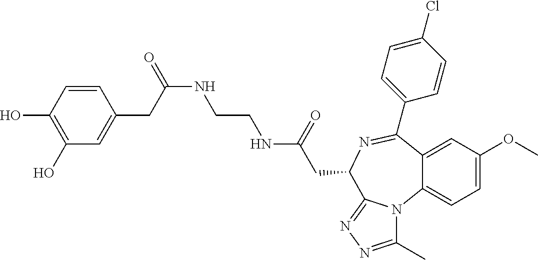 [SMILES: COc1ccc2c(c1)C(c1ccc(Cl)cc1)=N[C@@H](CC(=O)NCCNC(=O)Cc1ccc(O)c(O)c1)c1nnc(C)n1-2] |
| 332 | 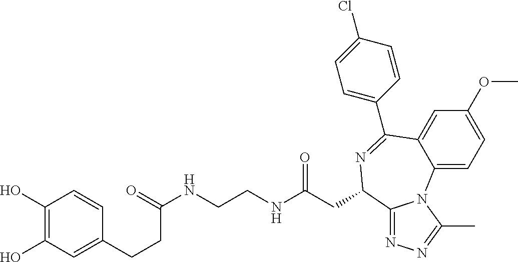 [SMILES: COc1ccc2c(c1)C(c1ccc(Cl)cc1)=N[C@@H](CC(=O)NCCNC(=O)CCc1ccc(O)c(O)c1)c1nnc(C)n1-2] |
| 333 | 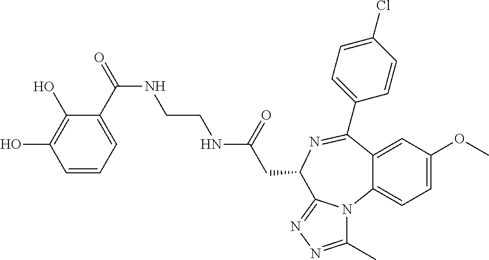 [SMILES: COc1ccc2c(c1)C(c1ccc(Cl)cc1)=N[C@@H](CC(=O)NCCNC(=O)c1cccc(O)c1O)c1nnc(C)n1-2] |
| 334 | 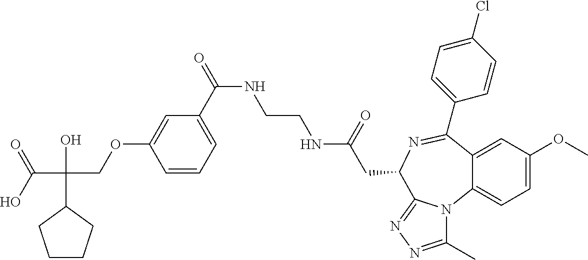 [SMILES: COc1ccc2c(c1)C(c1ccc(Cl)cc1)=N[C@@H](CC(=O)NCCNC(=O)c1cccc(OCC(O)(C(=O)O)C3CCCC3)c1)c1nnc(C)n1-2] |
| 335 | 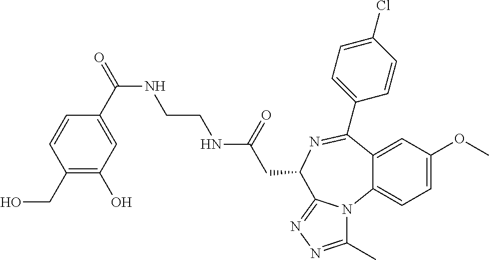 [SMILES: COc1ccc2c(c1)C(c1ccc(Cl)cc1)=N[C@@H](CC(=O)NCCNC(=O)c1ccc(CO)c(O)c1)c1nnc(C)n1-2] |
| 336 | 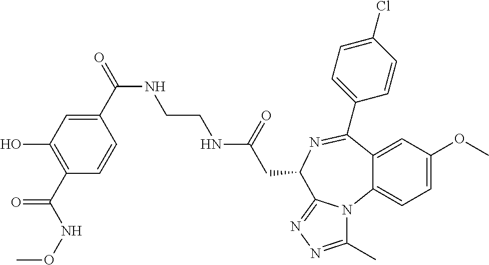 [SMILES: CONC(=O)c1ccc(C(=O)NCCNC(=O)C[C@@H]2N=C(c3ccc(Cl)cc3)c3cc(OC)ccc3-n3c(C)nnc32)cc1O] |
| 337 | 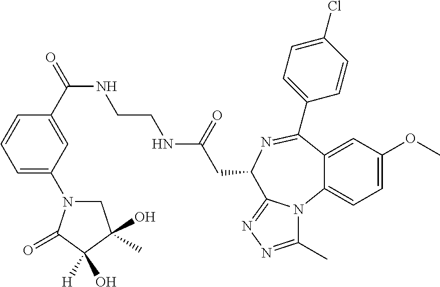 [SMILES: COc1ccc2c(c1)C(c1ccc(Cl)cc1)=N[C@@H](CC(=O)NCCNC(=O)c1cccc(N3C[C@@](C)(O)[C@@H](O)C3=O)c1)c1nnc(C)n1-2] |
| 338 | 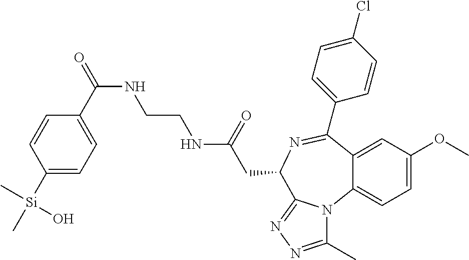 |
| 339 | 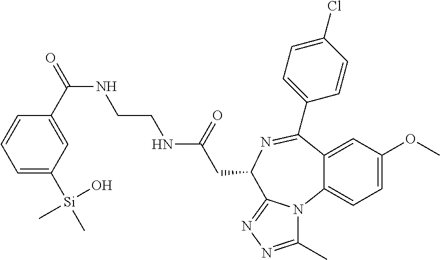 [SMILES: COc1ccc2c(c1)C(c1ccc(Cl)cc1)=N[C@@H](CC(=O)NCCNC(=O)c1cccc([Si](C)(C)O)c1)c1nnc(C)n1-2] |
| 340 | 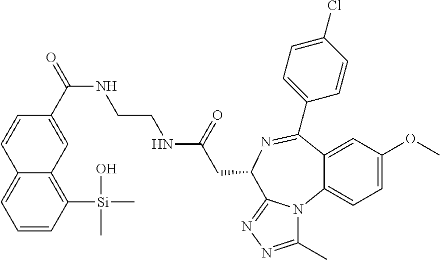 [SMILES: COc1ccc2c(c1)C(c1ccc(Cl)cc1)=N[C@@H](CC(=O)NCCNC(=O)c1ccc3cccc([Si](C)(C)O)c3c1)c1nnc(C)n1-2] |
| 341 | 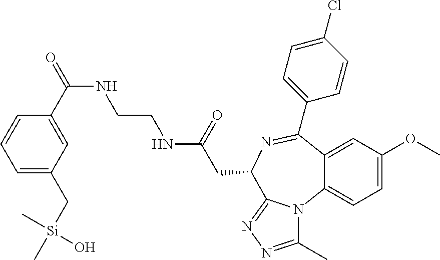 [SMILES: COc1ccc2c(c1)C(c1ccc(Cl)cc1)=N[C@@H](CC(=O)NCCNC(=O)c1cccc(C[Si](C)(C)O)c1)c1nnc(C)n1-2] |
| 342 | 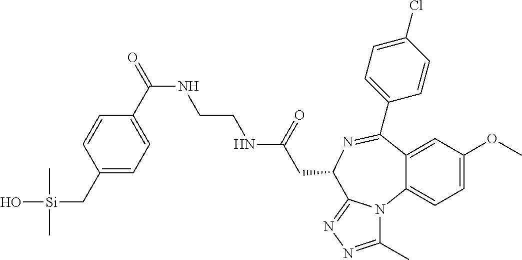 [SMILES: COc1ccc2c(c1)C(c1ccc(Cl)cc1)=N[C@@H](CC(=O)NCCNC(=O)c1ccc(C[Si](C)(C)O)cc1)c1nnc(C)n1-2] |
| 343 | 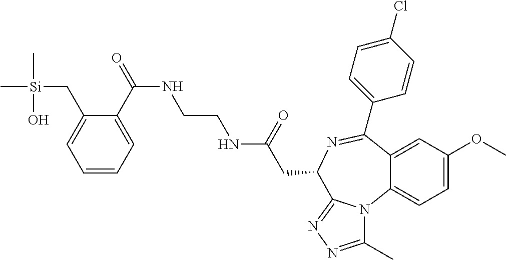 |
| 344 | 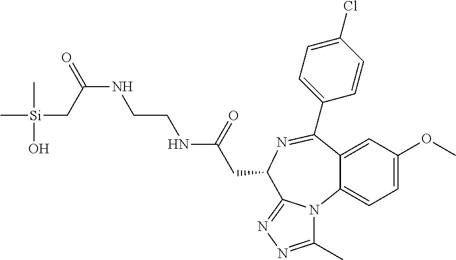 |
| 345 | 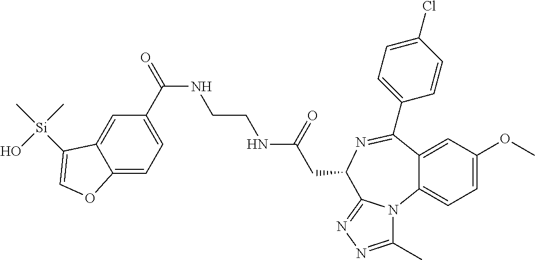 |
| 346 | 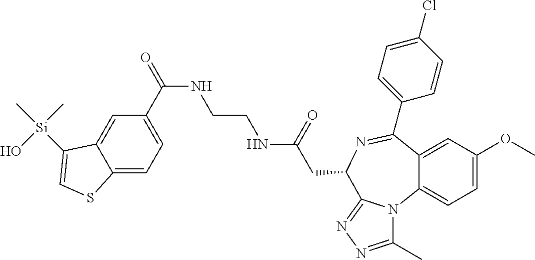 |
| 347 | 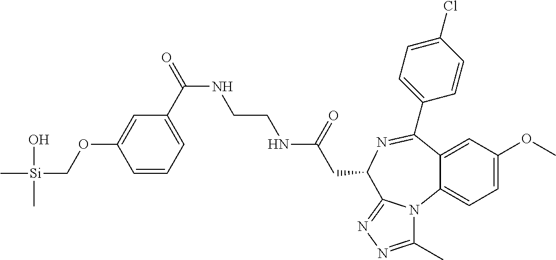 |
| 348 | 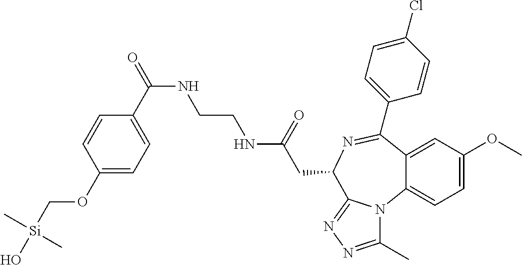 |
| 349 | 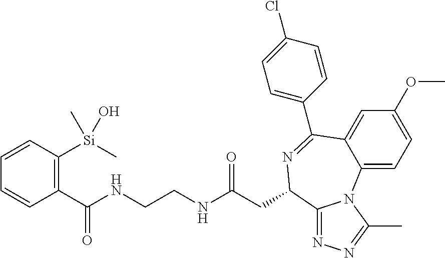 [SMILES: COc1ccc2c(c1)C(c1ccc(Cl)cc1)=N[C@@H](CC(=O)NCCNC(=O)c1ccccc1[Si](C)(C)O)c1nnc(C)n1-2] |
| 350 | 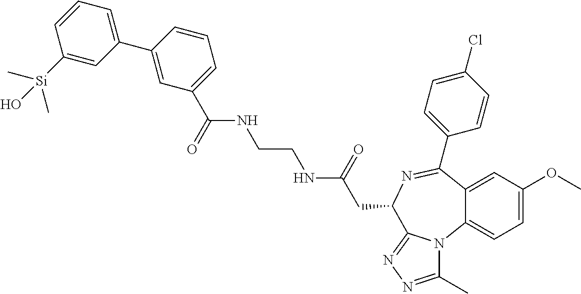 |
| 351 | 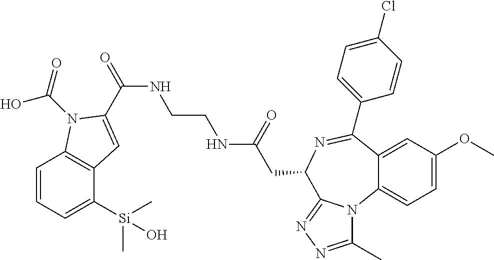 [SMILES: COc1ccc2c(c1)C(c1ccc(Cl)cc1)=N[C@@H](CC(=O)NCCNC(=O)c1cc3c([Si](C)(C)O)cccc3n1C(=O)O)c1nnc(C)n1-2] |

### Example 352

Table Z recites various contemplated monomers.

TABLE Z

| No. | Compound Structure |
|---|---|
| BRD-E-01 | 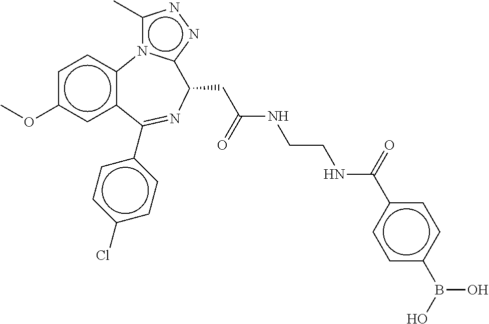 |
| BRD-E-02 | 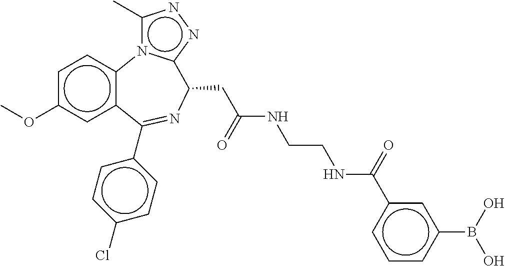 |
| BRD-E-03 | 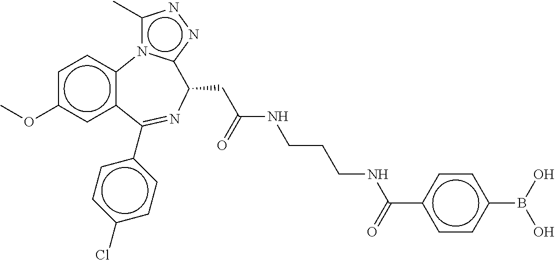 |
| BRD-E-04 | 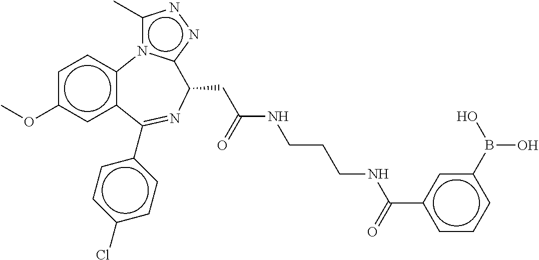 |
| BRD-E-05 | 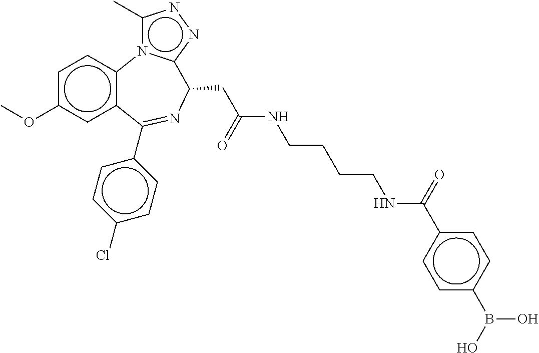 |
| BRD-E-06 | 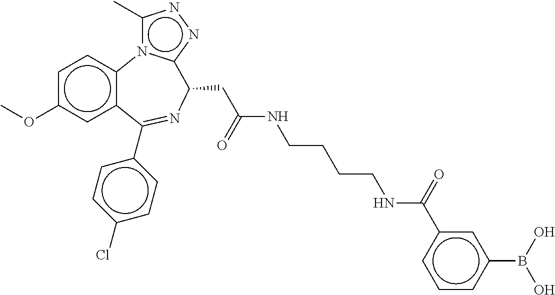 |
| BRD-E-07 | 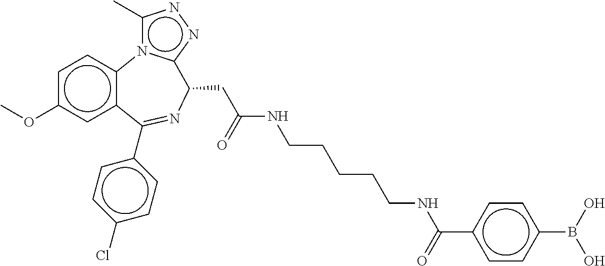 |
| BRD-E-08 | 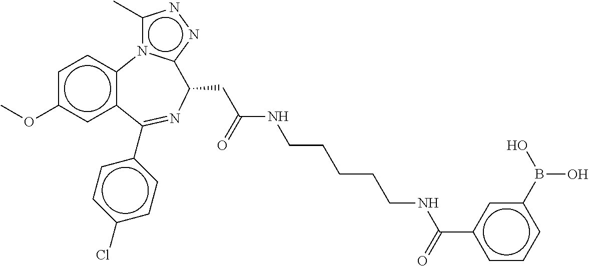 |
| BRD-N-01 | 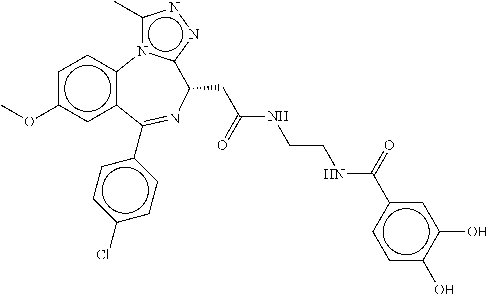 |
| BRD-N-02 | 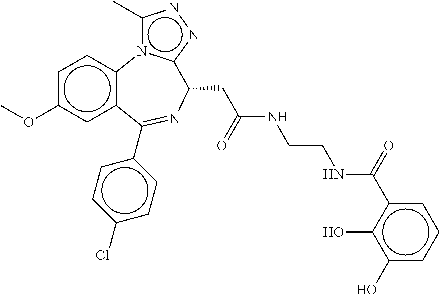 |
| BRD-N-03 | 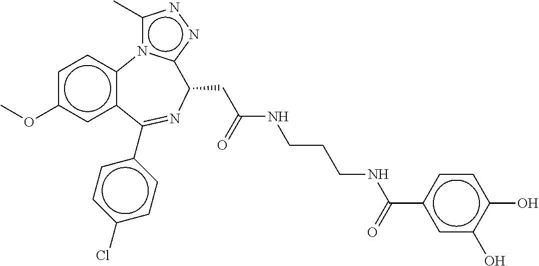 |
| BRD-N-04 | 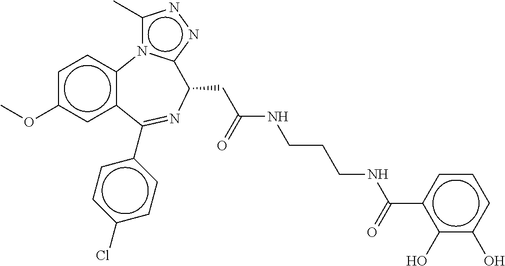 |
| BRD-N-05 | 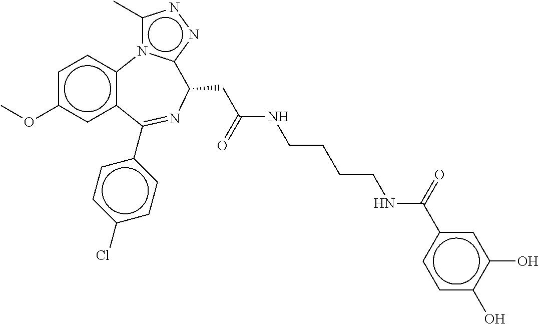 |
| BRD-N-06 | 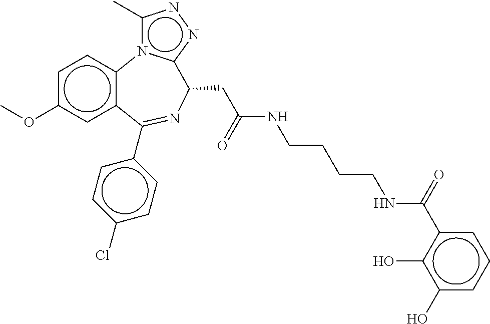 |
| BRD-N-07 | 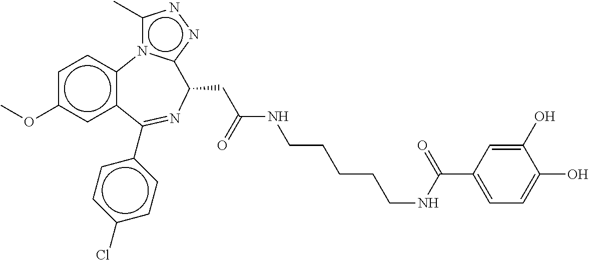 |
| BRD-N-08 | 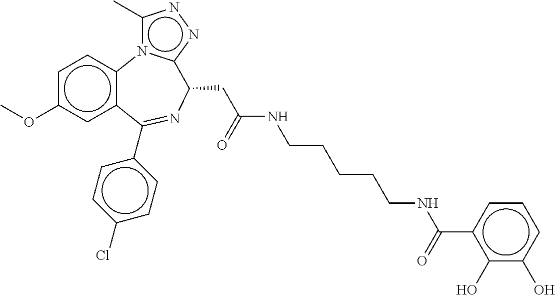 |
| BRD-S-01 | 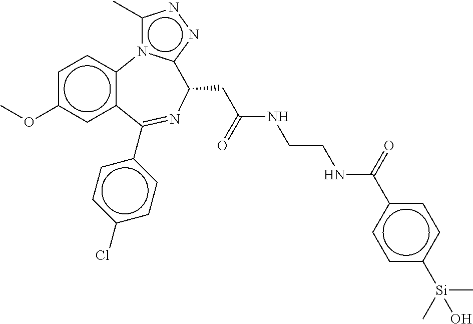 |
| BRD-S-02- variant | 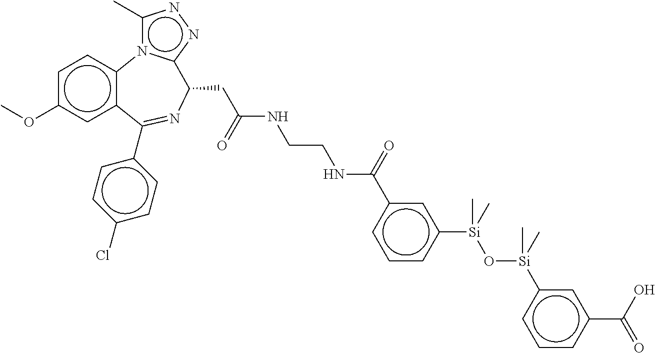 |
| BRD-S-02 | 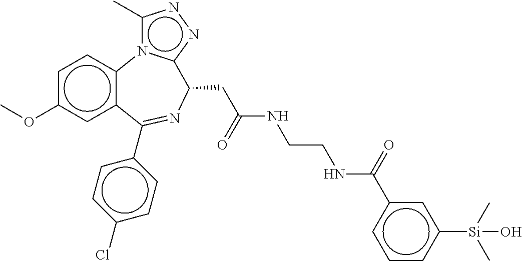 |
| BRD-S-03 | 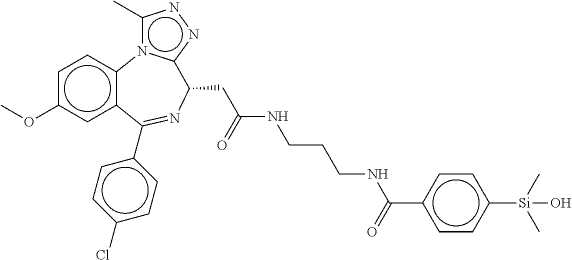 |
| BRD-S-04 | 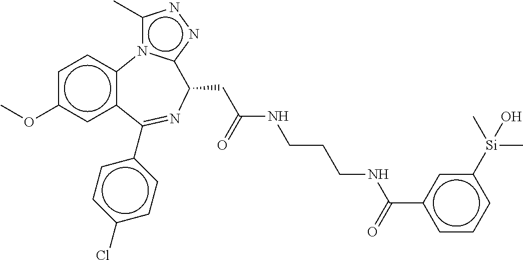 |
| BRD-S-05 | 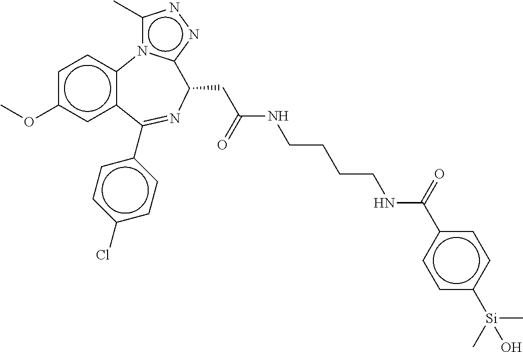 |
| BRD-S-06 | 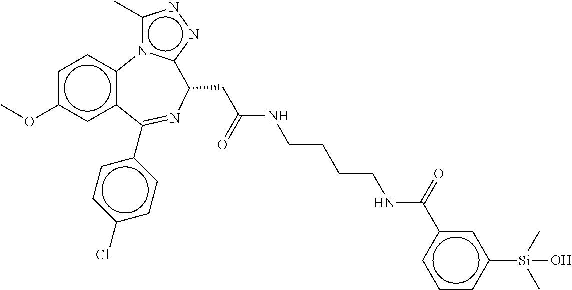 |
| BRD-S-07 | 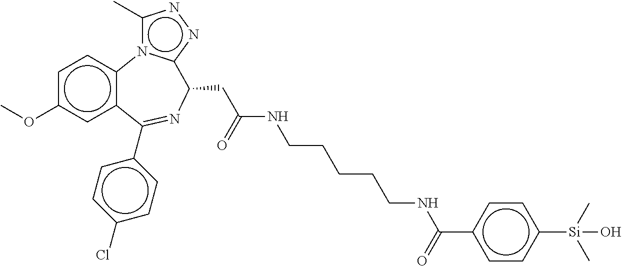 |
| BRD-S-08 | 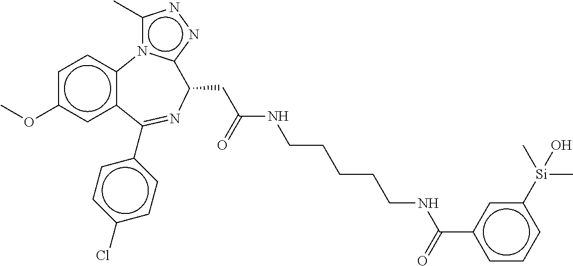 |
| BRD-S-03- variant | 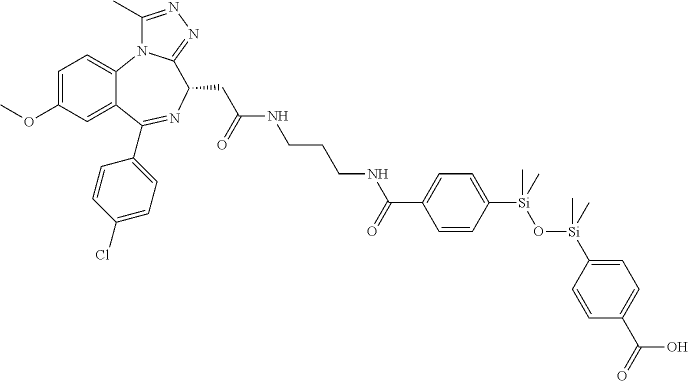 [SMILES: COc1ccc2c(c1)C(c1ccc(Cl)cc1)=N[C@@H](CC(=O)NCCCNC(=O)c1ccc([Si](C)(C)O[Si](C)(C)c3ccc(C(=O)O)cc3)cc1)c1nnc(C)n1-2] |
| BRD-S-05- variant | 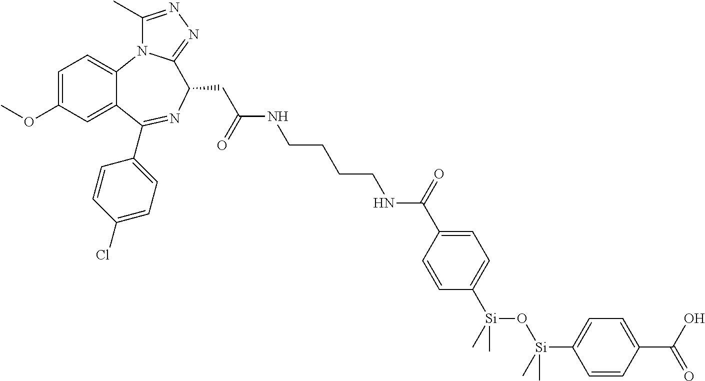 [SMILES: COc1ccc2c(c1)C(c1ccc(Cl)cc1)=N[C@@H](CC(=O)NCCCCNC(=O)c1ccc([Si](C)(C)O[Si](C)(C)c3ccc(C(=O)O)cc3)cc1)c1nnc(C)n1-2] |
| BRD-S-07 variant | 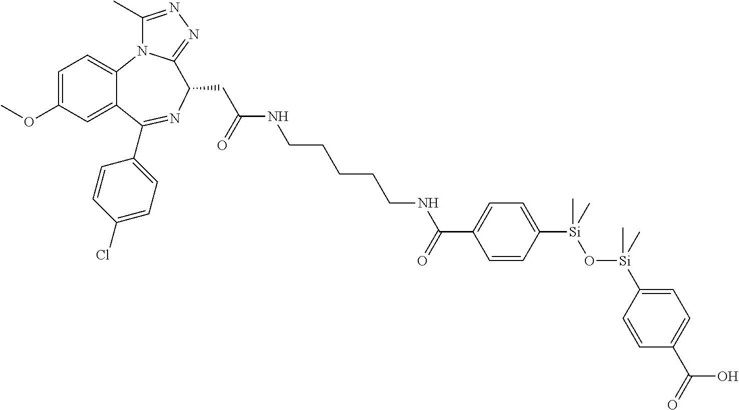 [SMILES: COc1ccc2c(c1)C(c1ccc(Cl)cc1)=N[C@@H](CC(=O)NCCCCCNC(=O)c1ccc([Si](C)(C)O[Si](C)(C)c3ccc(C(=O)O)cc3)cc1)c1nnc(C)n1-2] |
| Dimeric variant (BRD-S1,3,5,7) | 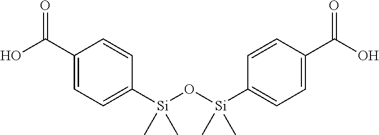 [SMILES: C[Si](C)(O[Si](C)(C)c1ccc(C(=O)O)cc1)c1ccc(C(=O)O)cc1] |
| BRD-N-42 | 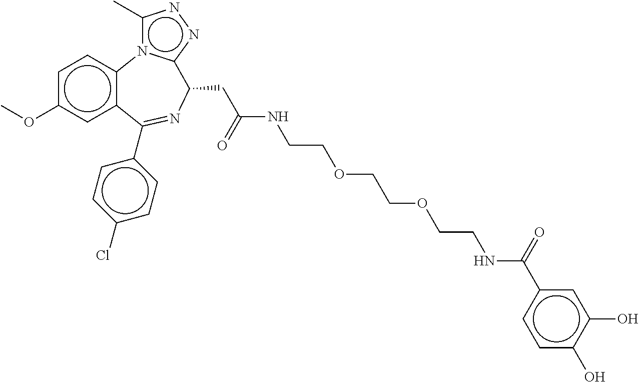 |
| BRD-N-43 | 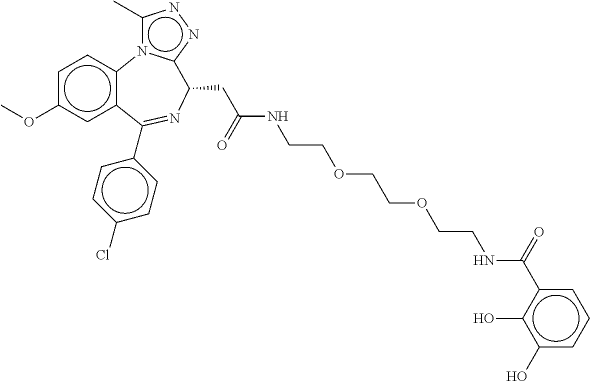 |
| BRD-N-44 | 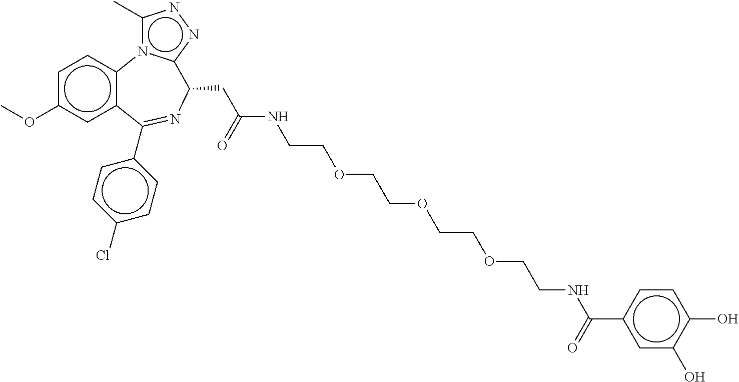 |
| BRD-N-45 | 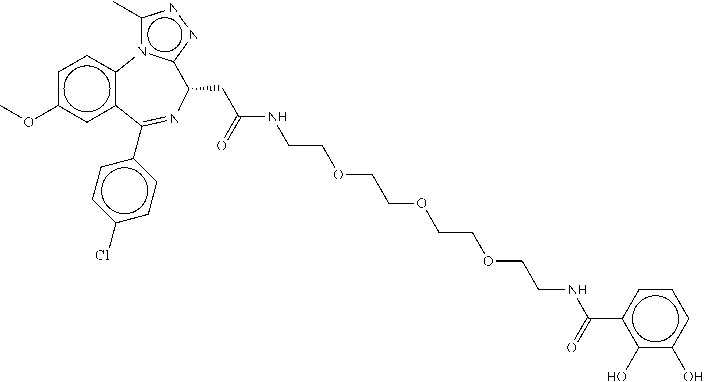 |
| BRD-E-45 | 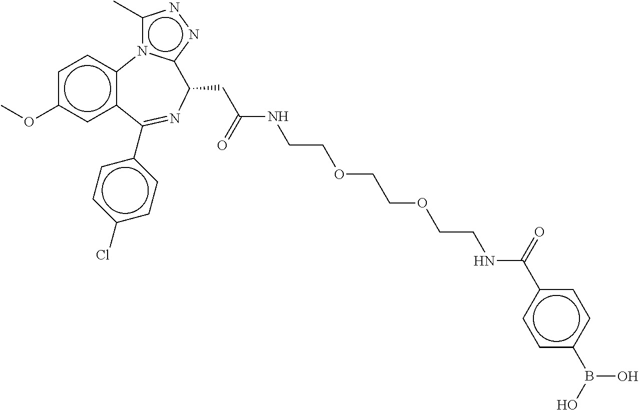 |
| BRD-E-46 | 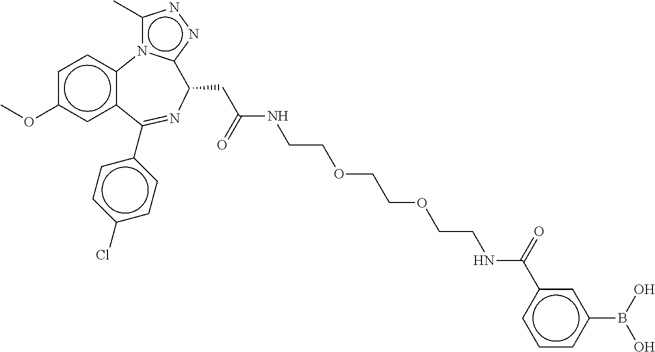 |
| | 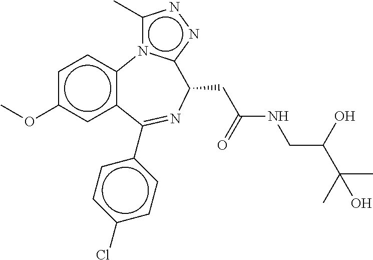 |
| | 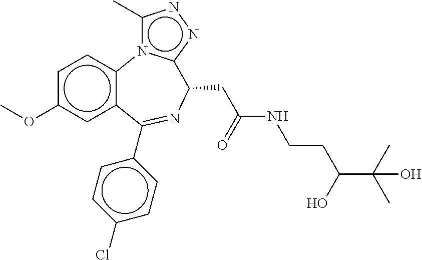 |
| | 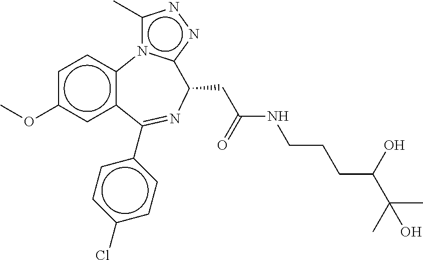 |
| BRD-E-27 | 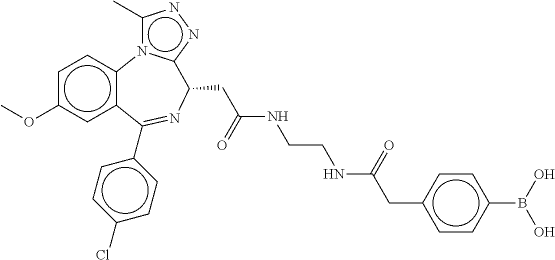 |
| BRD-E-29 | 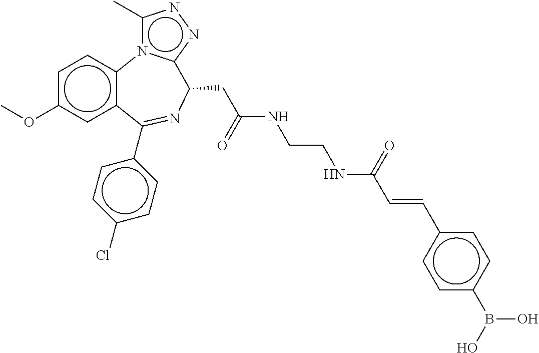 |
| BRD-E-30 | 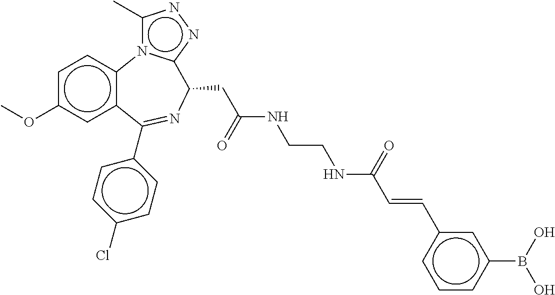 |
| BRD-E-37 | 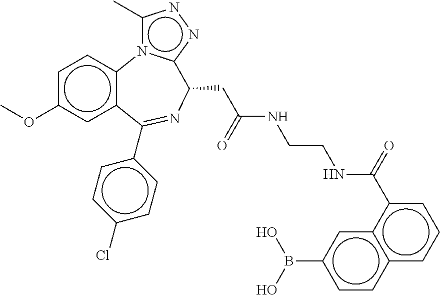 |
| BRD-E-31 | 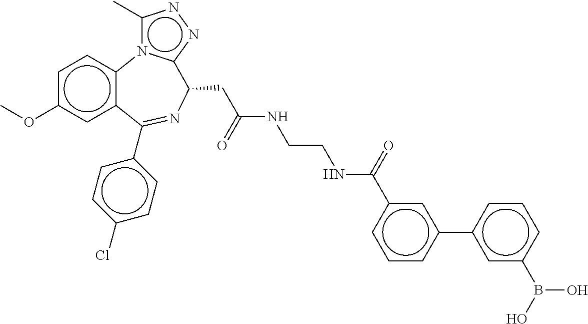 |
| BRD-E-36 | 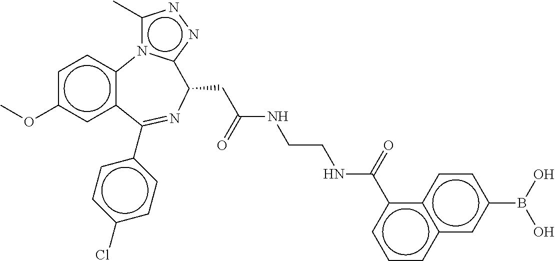 |
| BRD-E-38 | 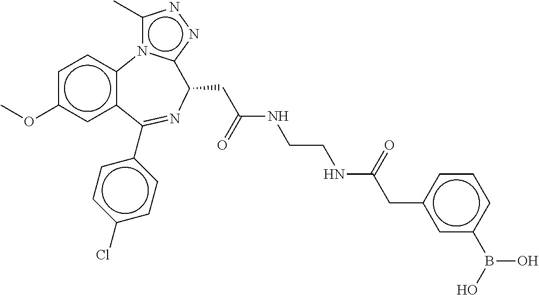 |
| BRD-E-28 | 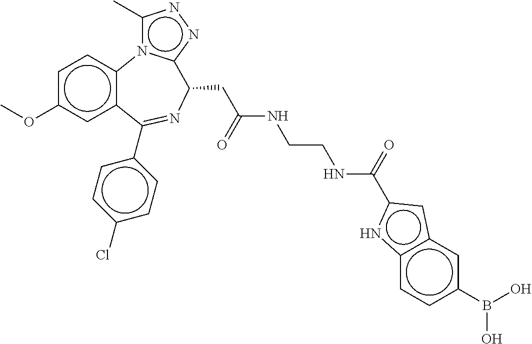 |
| BRD-E-32 | 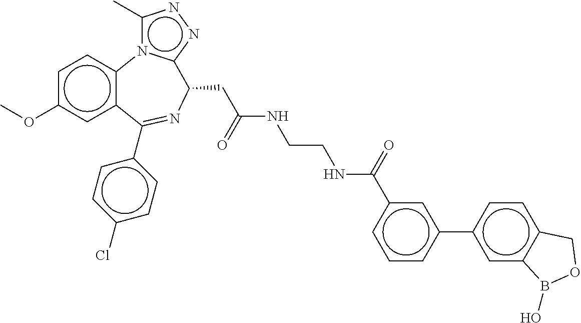 |
| BRD-E-33 | 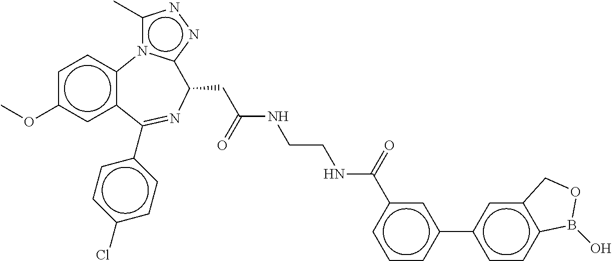 |
| BRD-E-34 | 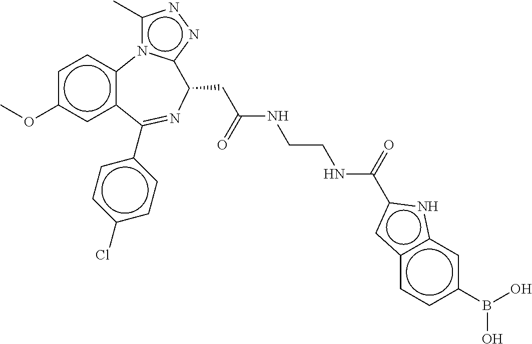 |
| BRD-E-35 | 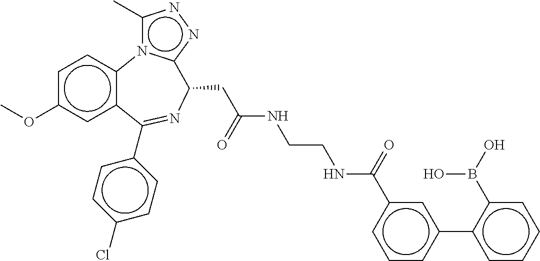 |
| BRD-N-31 | 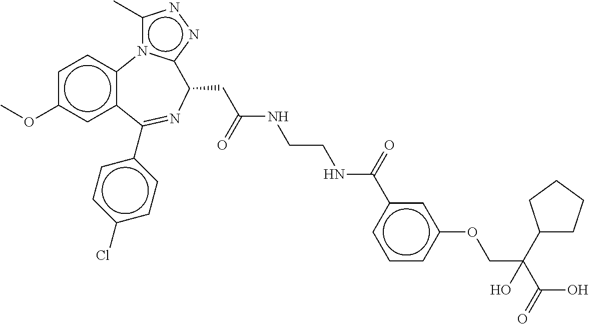 |
| BRD-N-30 | 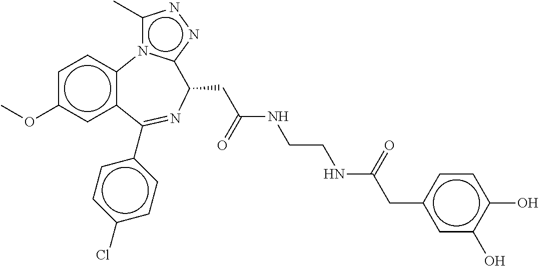 |
| BRD-N-35 | 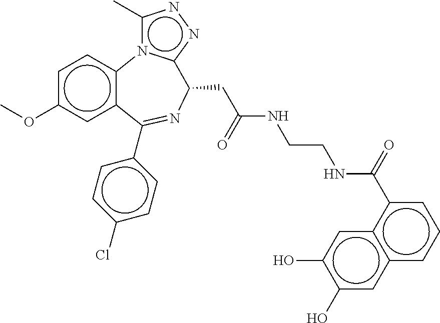 |
| BRD-N-34 | 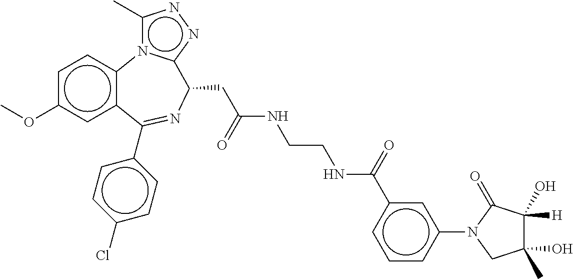 |
| BRD-N-32 | 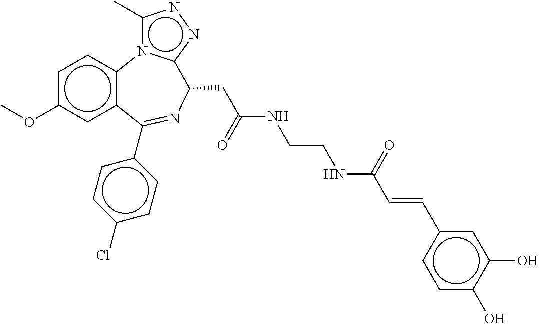 |
| BRD-N-33 | 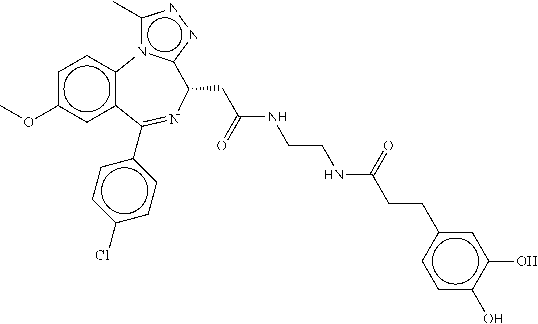 |
| BRD-N-37 | 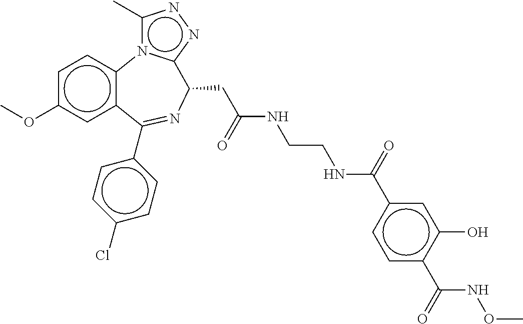 |
| BRD-N-36 | 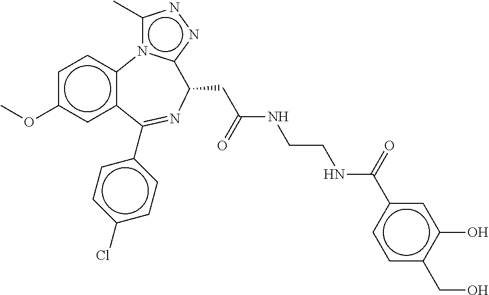 |
| BRD-S-24 | 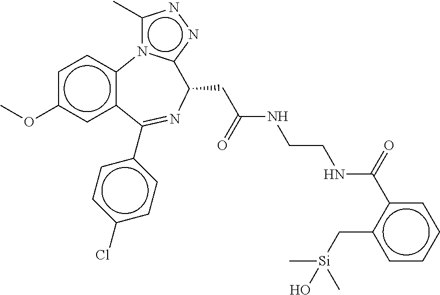 |
| BRD-S-18 | 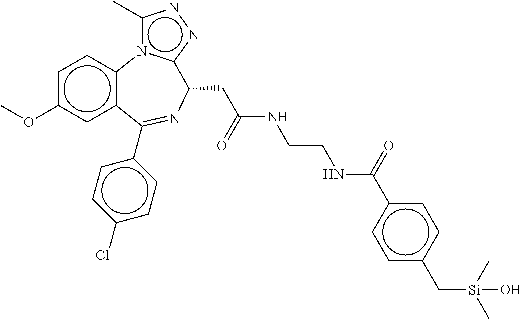 |
| BRD-S-19 | 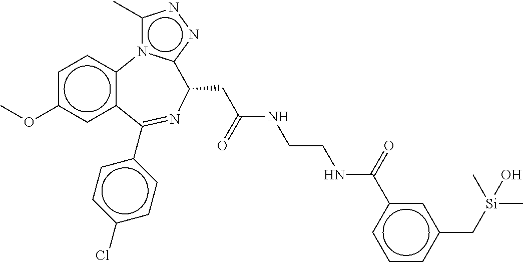 |
| BRD-S-21 | 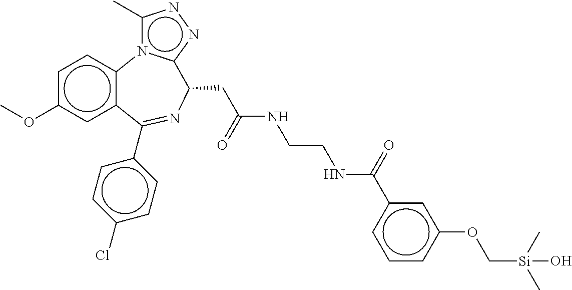 |
| BRD-S-20 | 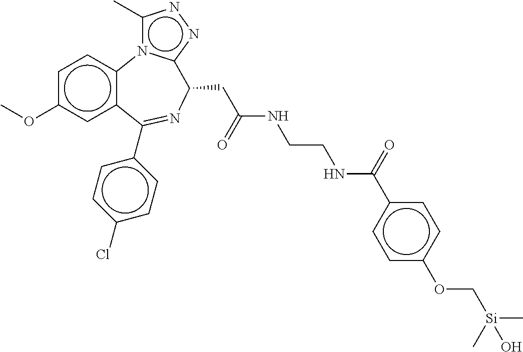 |
| BRD-S-22 | 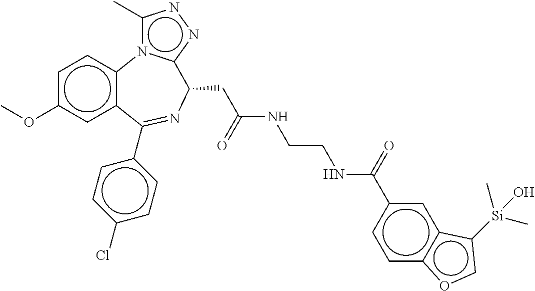 |
| BRD-S-23 | 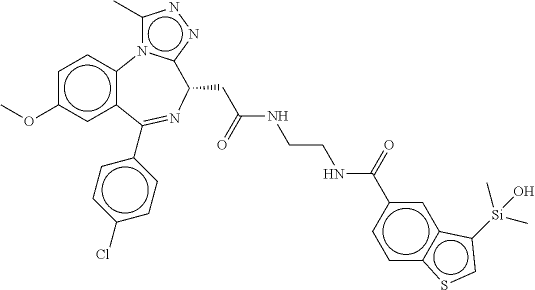 |
| BRD-S-25 | 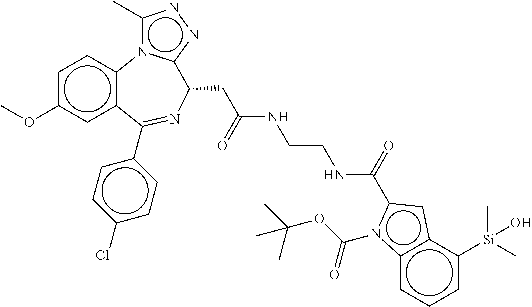 |
| BRD-S-17 | 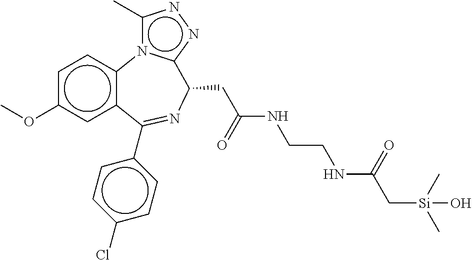 |
| BRD-S-26 | 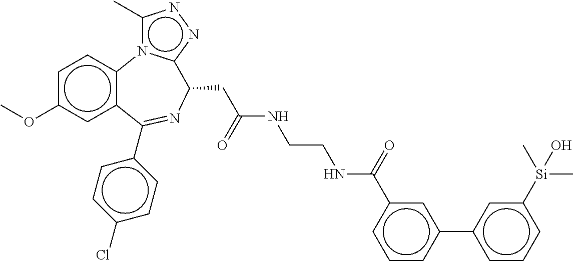 |
| BRD-S-27 | 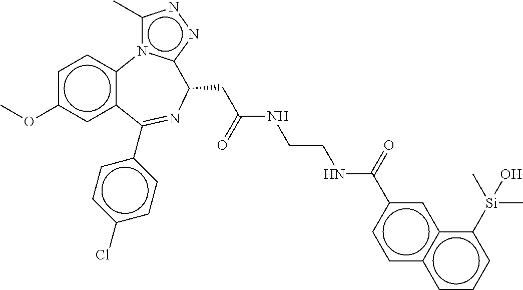 |
| BRD-E-09 | 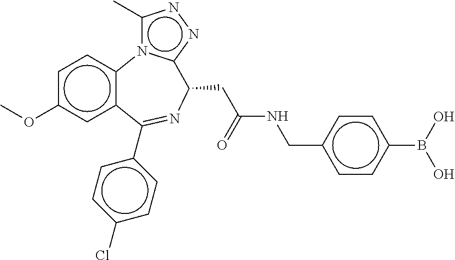 |
| BRD-E-10 | 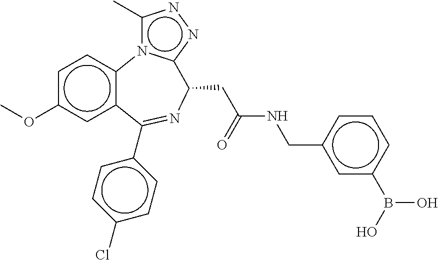 |
| BRD-N-09 | 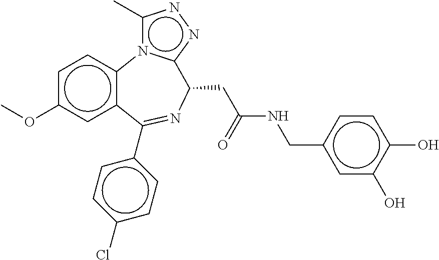 |
| BRD-N-10 | 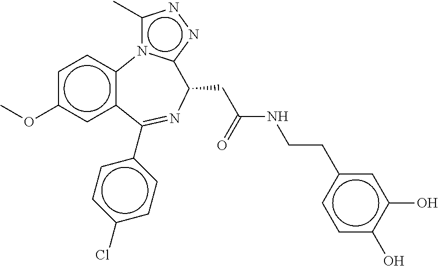 |
| BRD-N-11 | 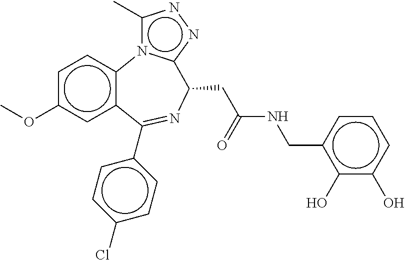 |
| BRD-N-12 | 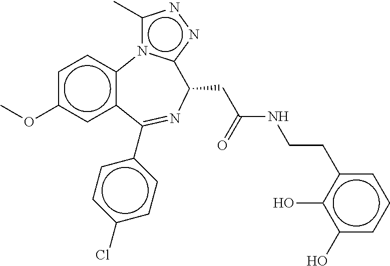 |
| BRD-N-13 | 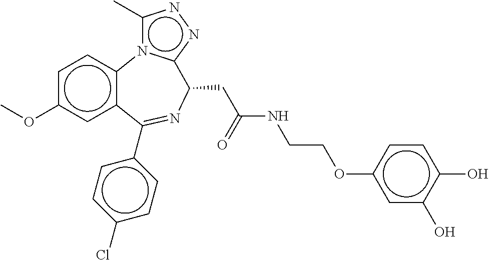 |
| BRD-N-14 | 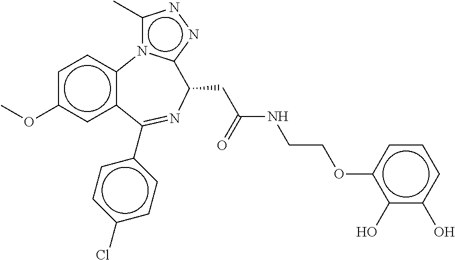 |
| BRD-E-11 | 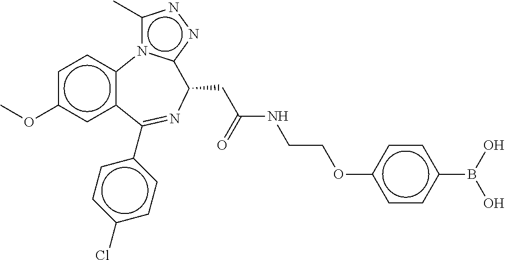 |
| BRD-S-09 | 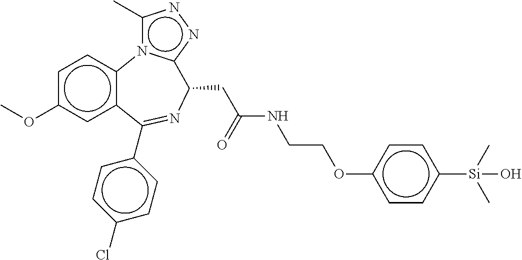 |
| BRD-E-12 | 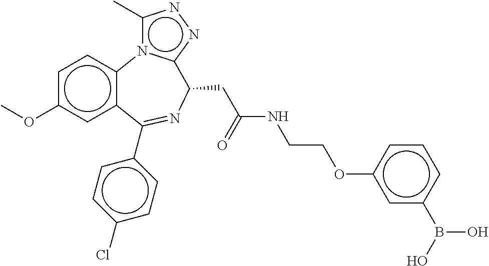 |
| BRD-S-10 | 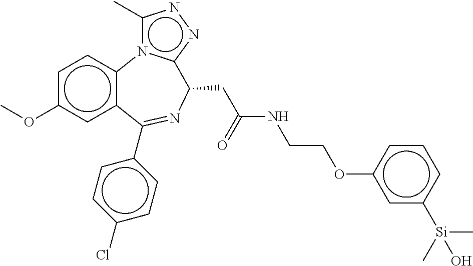 |
| BRD-E-13 | 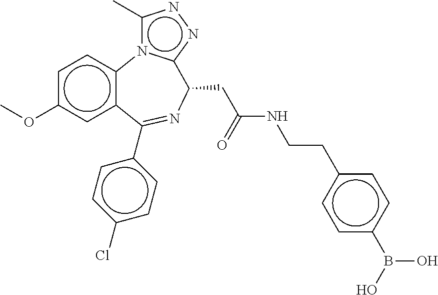 |
| BRD-S-11 | 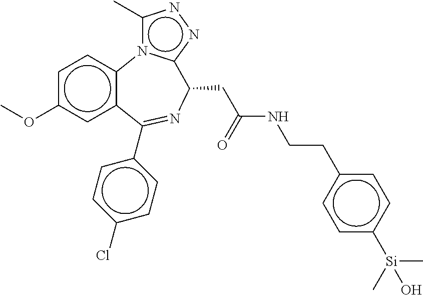 |
| BRD-E-14 | 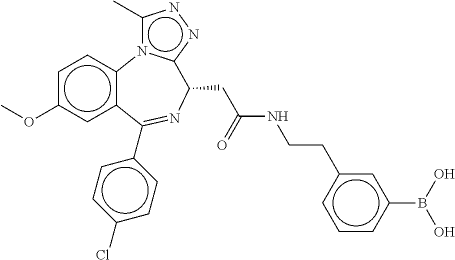 |
| BRD-S-12 | 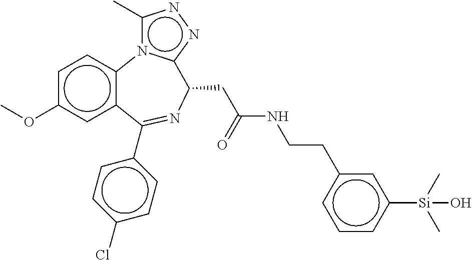 |
| BRD-S-13 | 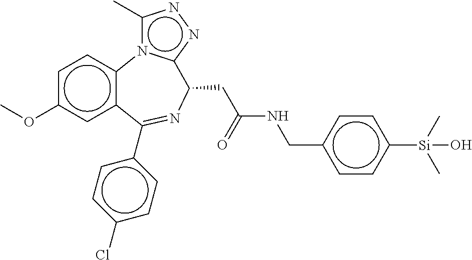 |
| BRD-S-14 | 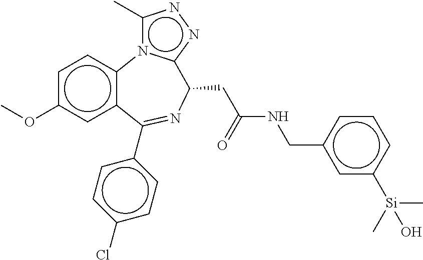 |
| BRD-S-15 | 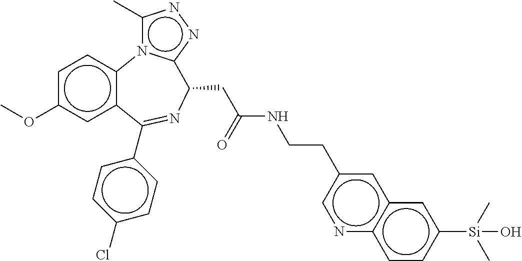 |
| BRD-S-16 | 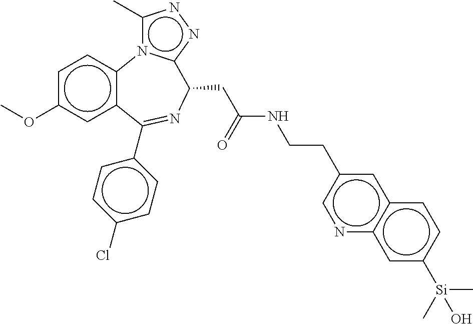 |
| BRD-N-15 | 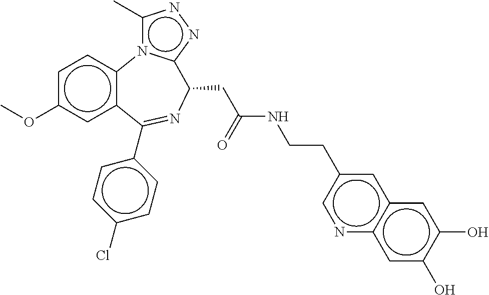 |
| BRD-E-15 | 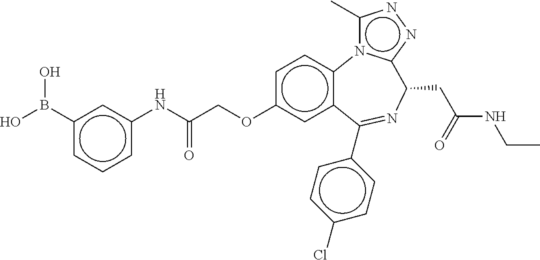 |
| BRD-E-16 | 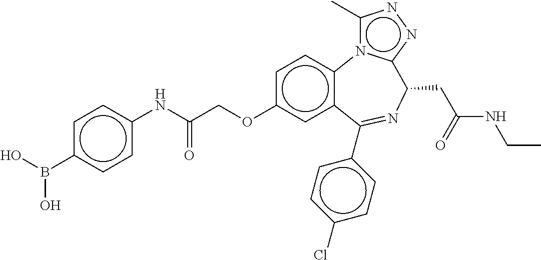 |
| BRD-E-17 | 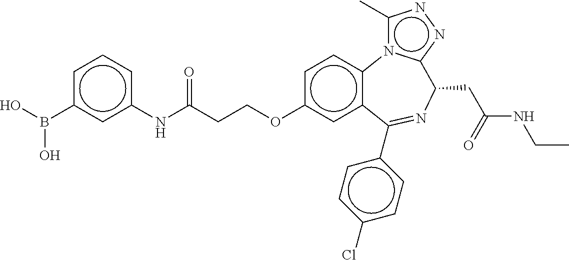 |
| BRD-E-18 | 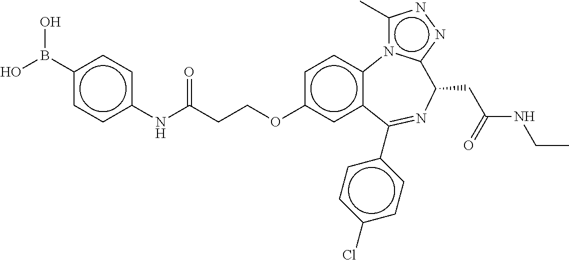 |
| BRD-E-19 | 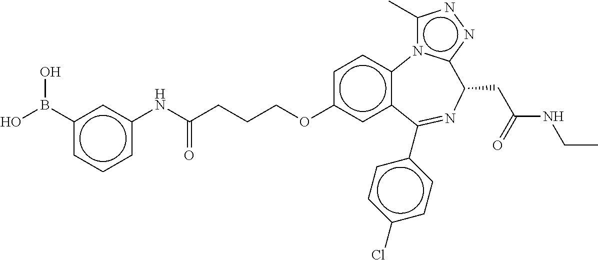 |
| BRD-E-20 | 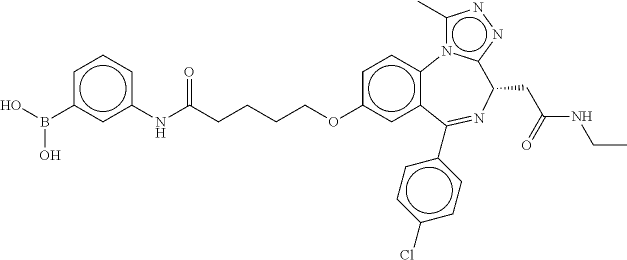 |
| BRD-E-21 | 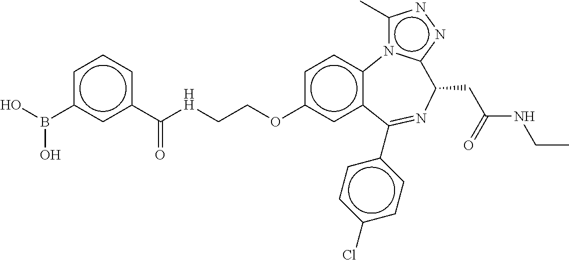 |
| BRD-E-22 | 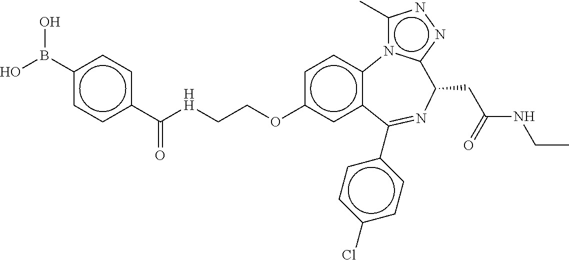 |
| BRD-E-23 | 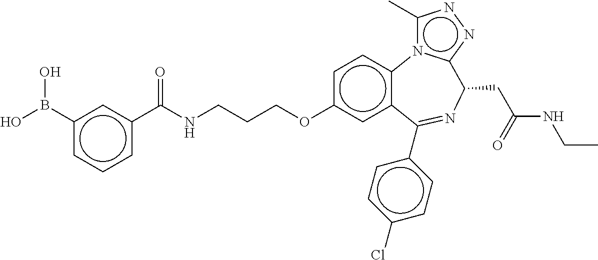 |
| BRD-E-24 | 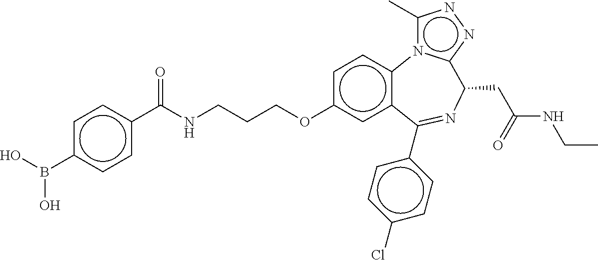 |
| BRD-E-25 | 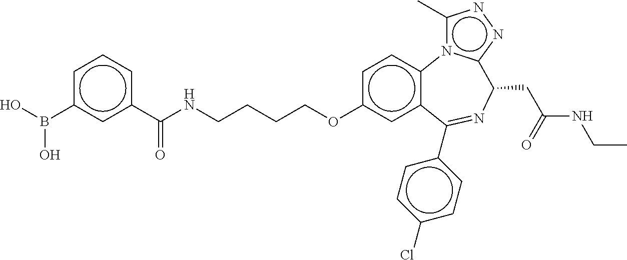 |
| BRD-E-26 | 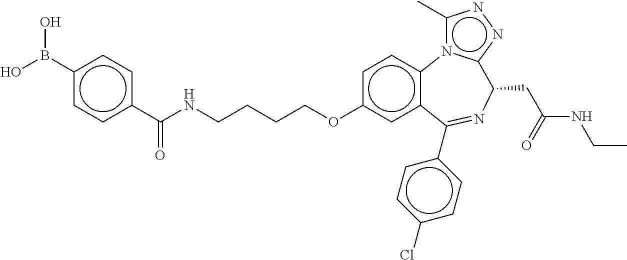 |
| BRD-N-16 | 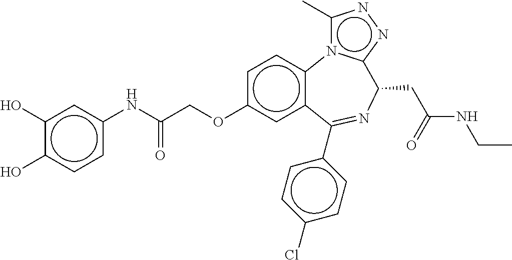 |
| BRD-N-17 | 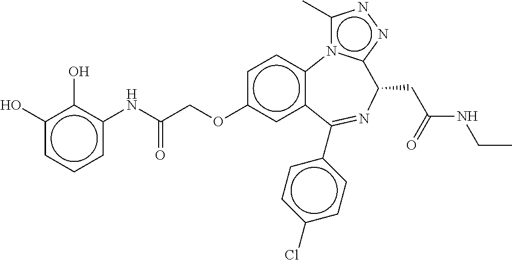 |
| BRD-N-18 | 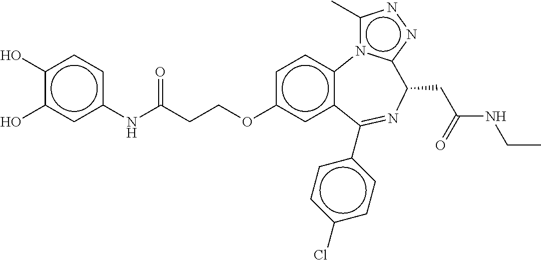 |
| BRD-N-19 | 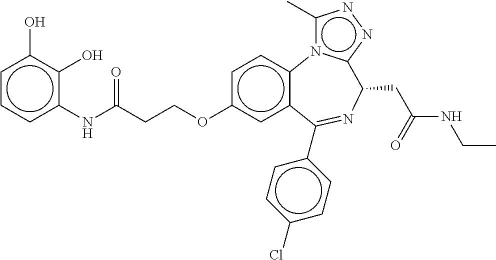 |
| BRD-N-20 | 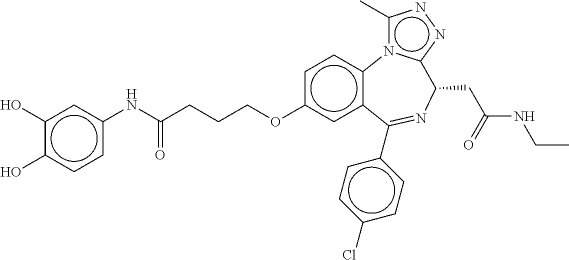 |
| BRD-N-21 | 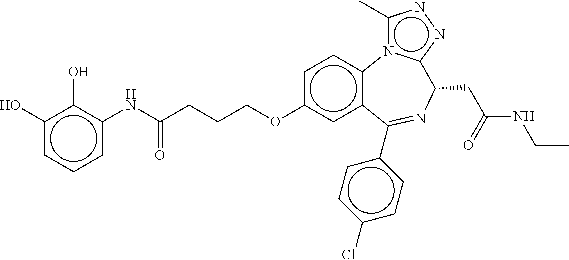 |
| BRD-N-22 | 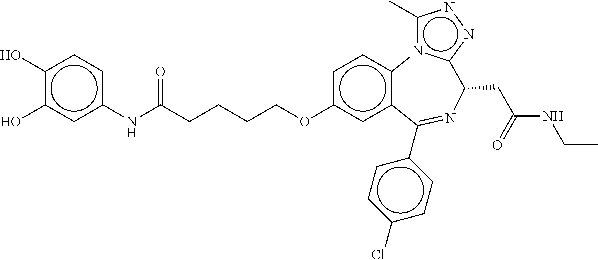 |
| BRD-N-23 | 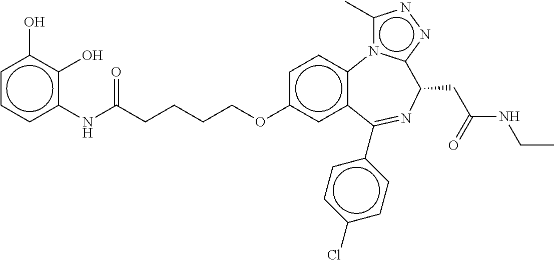 |
| BRD-N-24 | 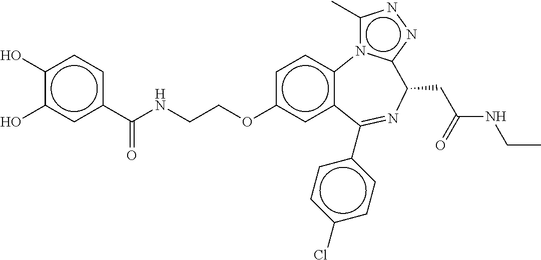 |
| BRD-N-25 | 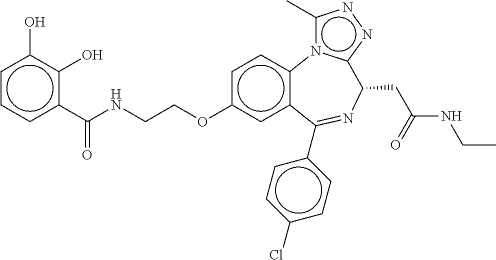 |
| BRD-N-26 | 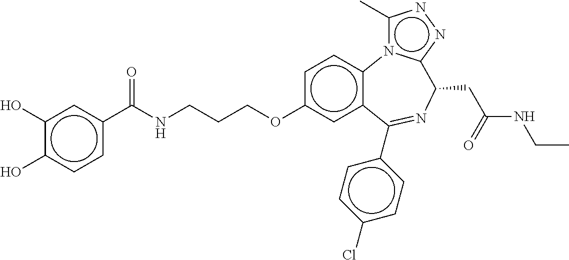 |
| BRD-N-27 | 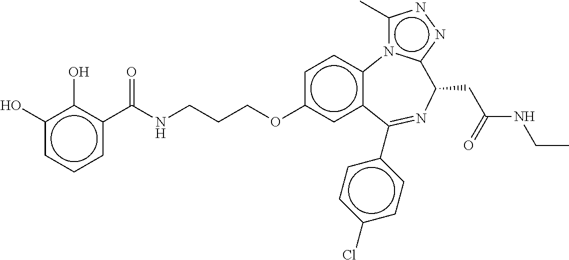 |
| BRD-N-28 | 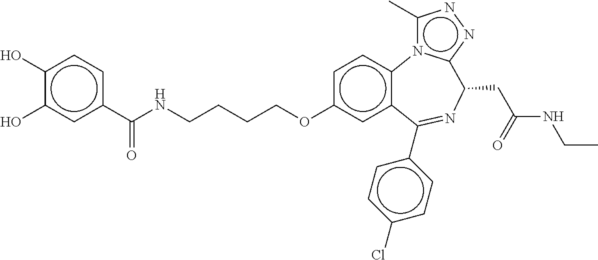 |
| BRD-N-29 | 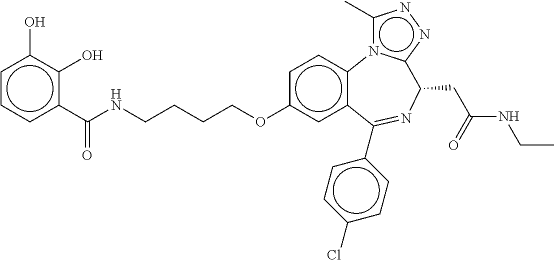 |
| BRD-E-39 | 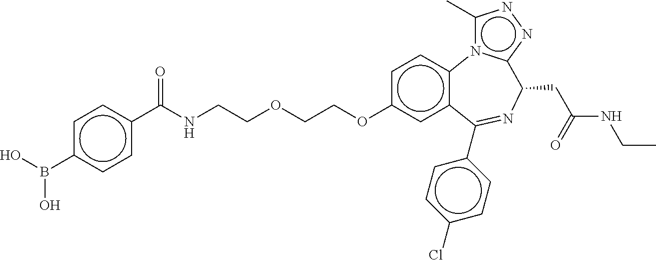 |
| BRD-E-40 | 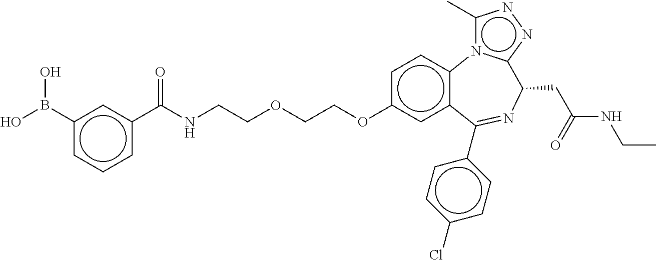 |
| BRD-N-38 | 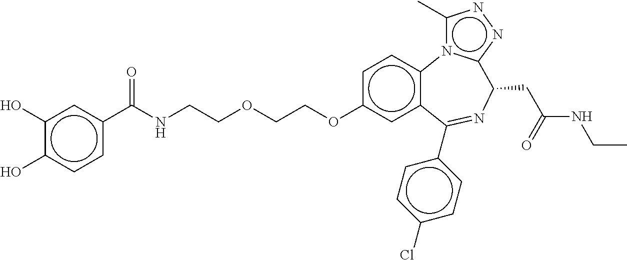 |
| BRD-N-39 | 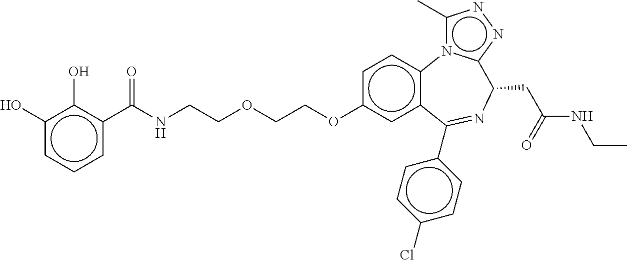 |
| BRD-E-41 | 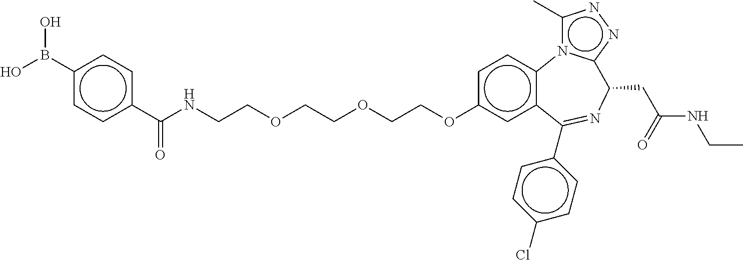 |
| BRD-E-42 | 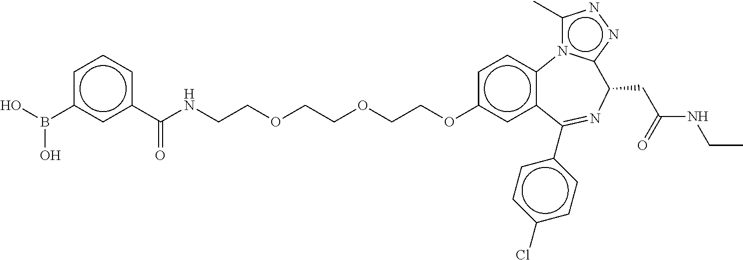 |
| BRD-N-40 | 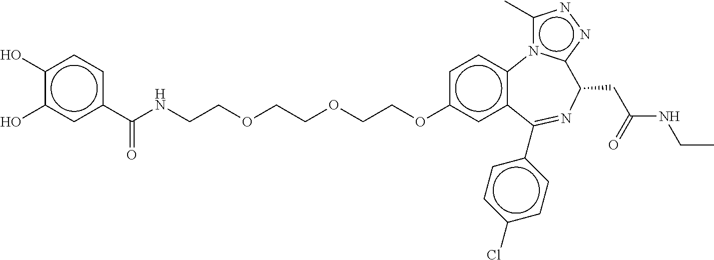 |
| BRD-N-41 | 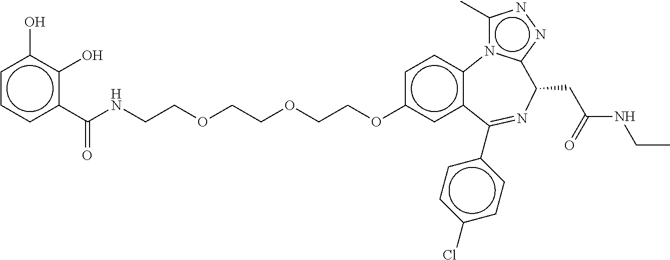 |
| BRD-E-43 | 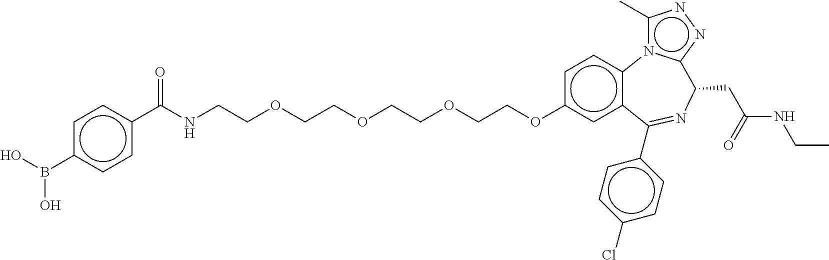 |
| BRD-E-44 | 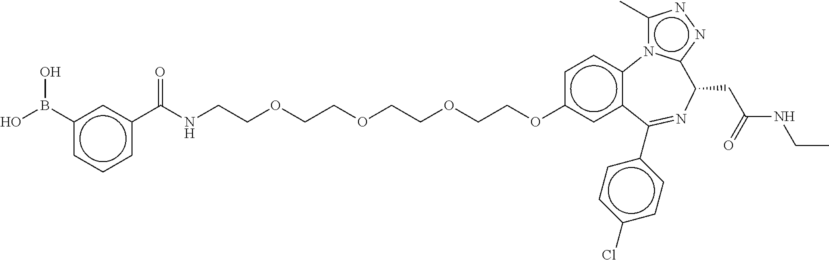 |
| | 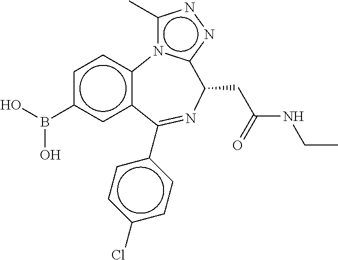 |
| | 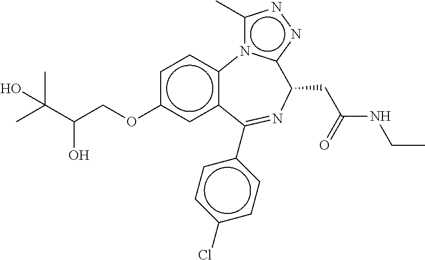 |
| | 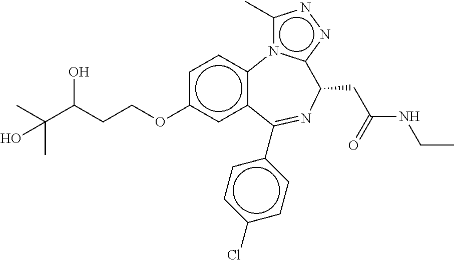 |
| | 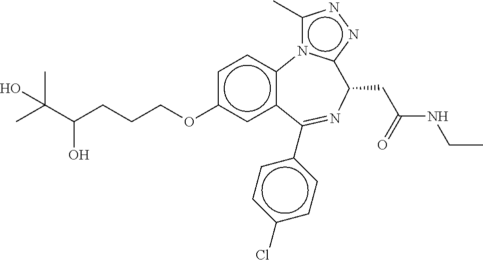 |
| | 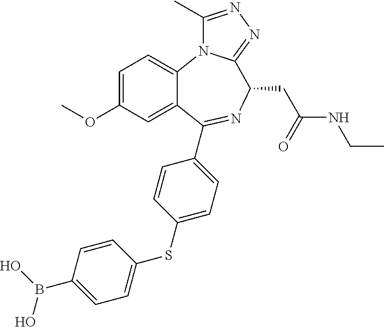 [SMILES: CCNC(=O)C[C@@H]1N=C(c2ccc(Sc3ccc(B(O)O)cc3)cc2)c2cc(OC)ccc2-n2c(C)nnc21] |
| | 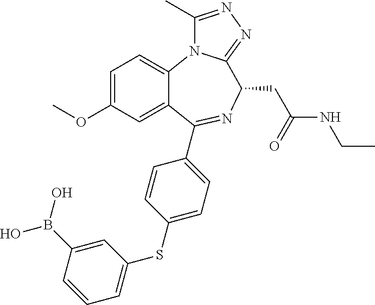 [SMILES: CCNC(=O)C[C@@H]1N=C(c2ccc(Sc3cccc(B(O)O)c3)cc2)c2cc(OC)ccc2-n2c(C)nnc21] |
| | 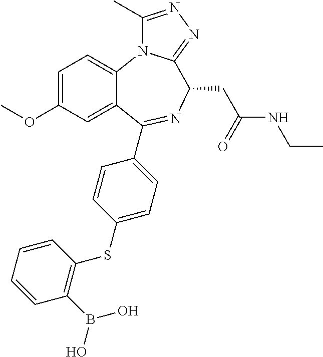 [SMILES: CCNC(=O)C[C@@H]1N=C(c2ccc(Sc3ccccc3B(O)O)cc2)c2cc(OC)ccc2-n2c(C)nnc21] |
| | 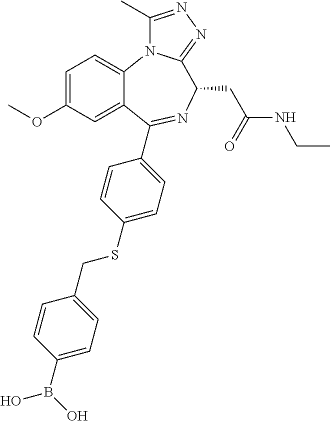 [SMILES: CCNC(=O)C[C@@H]1N=C(c2ccc(SCc3ccc(B(O)O)cc3)cc2)c2cc(OC)ccc2-n2c(C)nnc21] |
| | 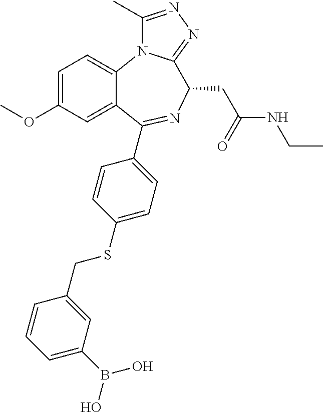 [SMILES: CCNC(=O)C[C@@H]1N=C(c2ccc(SCc3cccc(B(O)O)c3)cc2)c2cc(OC)ccc2-n2c(C)nnc21] |
| | 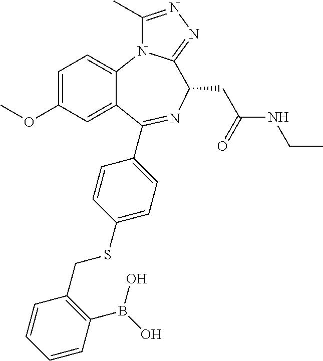 [SMILES: CCNC(=O)C[C@@H]1N=C(c2ccc(SCc3ccccc3B(O)O)cc2)c2cc(OC)ccc2-n2c(C)nnc21] |
| BRD-E-57 | 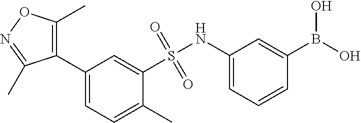 [SMILES: Cc1ccc(-c2c(C)noc2C)cc1S(=O)(=O)Nc1cccc(B(O)O)c1] |
| | 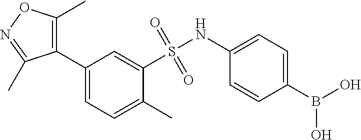 [SMILES: Cc1ccc(-c2c(C)noc2C)cc1S(=O)(=O)Nc1ccc(B(O)O)cc1] |
| | 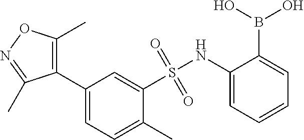 [SMILES: Cc1ccc(-c2c(C)noc2C)cc1S(=O)(=O)Nc1ccccc1B(O)O] |
| | 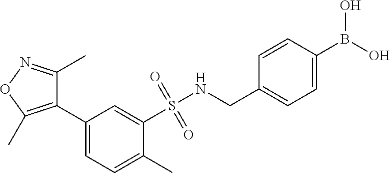 [SMILES: Cc1ccc(-c2c(C)noc2C)cc1S(=O)(=O)NCc1ccc(B(O)O)cc1] |
| | 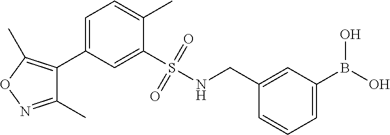 [SMILES: Cc1ccc(-c2c(C)noc2C)cc1S(=O)(=O)NCc1cccc(B(O)O)c1] |
| | 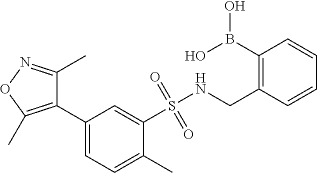 [SMILES: Cc1ccc(-c2c(C)noc2C)cc1S(=O)(=O)NCc1ccccc1B(O)O] |
| | 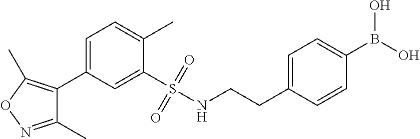 [SMILES: Cc1ccc(-c2c(C)noc2C)cc1S(=O)(=O)NCCc1ccc(B(O)O)cc1] |
| | 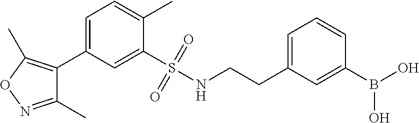 [SMILES: Cc1ccc(-c2c(C)noc2C)cc1S(=O)(=O)NCCc1cccc(B(O)O)c1] |
| | 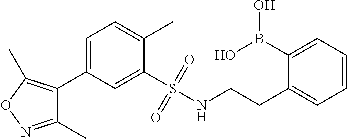 [SMILES: Cc1ccc(-c2c(C)noc2C)cc1S(=O)(=O)NCCc1ccccc1B(O)O] |
| | 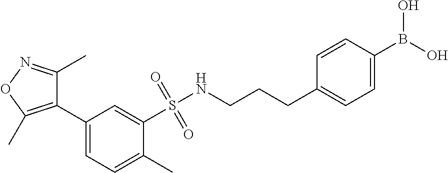 [SMILES: Cc1ccc(-c2c(C)noc2C)cc1S(=O)(=O)NCCCc1ccc(B(O)O)cc1] |
| | 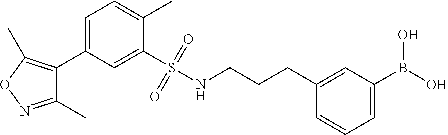 [SMILES: Cc1ccc(-c2c(C)noc2C)cc1S(=O)(=O)NCCCc1cccc(B(O)O)c1] |
| | 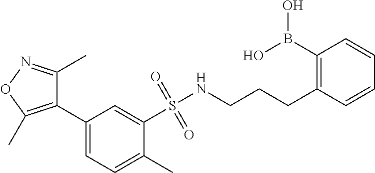 [SMILES: Cc1ccc(-c2c(C)noc2C)cc1S(=O)(=O)NCCCc1ccccc1B(O)O] |
| | 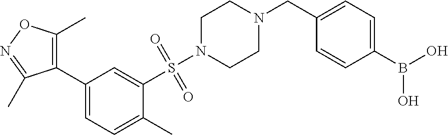 [SMILES: Cc1ccc(-c2c(C)noc2C)cc1S(=O)(=O)N1CCN(Cc2ccc(B(O)O)cc2)CC1] |
| | 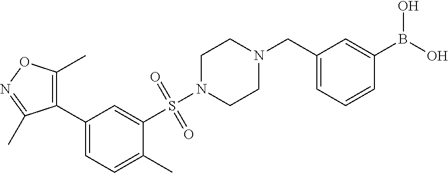 [SMILES: Cc1ccc(-c2c(C)noc2C)cc1S(=O)(=O)N1CCN(Cc2cccc(B(O)O)c2)CC1] |
| | 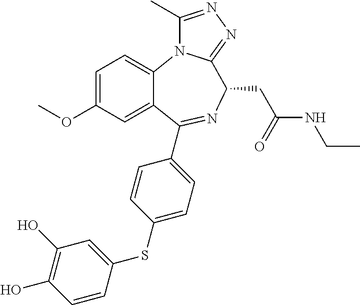 [SMILES: CCNC(=O)C[C@@H]1N=C(c2ccc(Sc3ccc(O)c(O)c3)cc2)c2cc(OC)ccc2-n2c(C)nnc21] |
| | 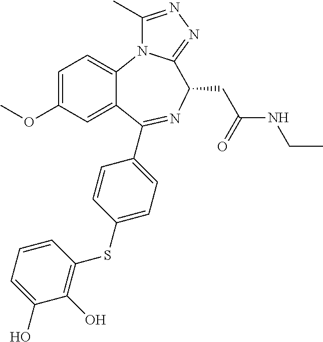 [SMILES: CCNC(=O)C[C@@H]1N=C(c2ccc(Sc3cccc(O)c3O)cc2)c2cc(OC)ccc2-n2c(C)nnc21] |
| | 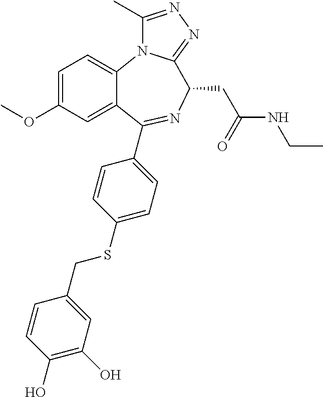 [SMILES: CCNC(=O)C[C@@H]1N=C(c2ccc(SCc3ccc(O)c(O)c3)cc2)c2cc(OC)ccc2-n2c(C)nnc21] |
| | 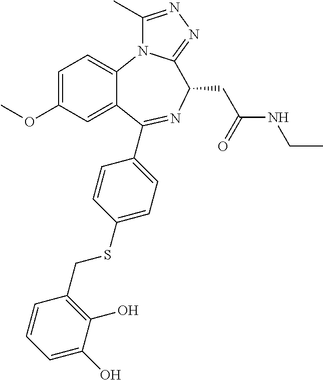 [SMILES: CCNC(=O)C[C@@H]1N=C(c2ccc(SCc3cccc(O)c3O)cc2)c2cc(OC)ccc2-n2c(C)nnc21] |
| | 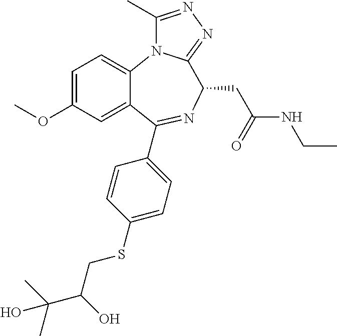 [SMILES: CCNC(=O)C[C@@H]1N=C(c2ccc(SCC(O)C(C)(C)O)cc2)c2cc(OC)ccc2-n2c(C)nnc21] |
| | 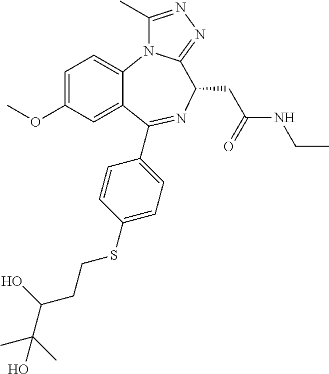 [SMILES: CCNC(=O)C[C@@H]1N=C(c2ccc(SCCC(O)C(C)(C)O)cc2)c2cc(OC)ccc2-n2c(C)nnc21] |
| | 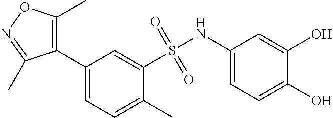 [SMILES: Cc1ccc(-c2c(C)noc2C)cc1S(=O)(=O)Nc1ccc(O)c(O)c1] |
| | 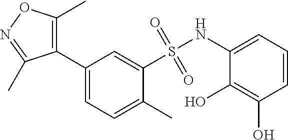 [SMILES: Cc1ccc(-c2c(C)noc2C)cc1S(=O)(=O)Nc1cccc(O)c1O] |
| | 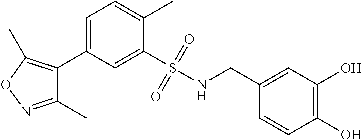 [SMILES: Cc1ccc(-c2c(C)noc2C)cc1S(=O)(=O)NCc1ccc(O)c(O)c1] |
| | 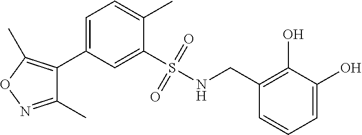 [SMILES: Cc1ccc(-c2c(C)noc2C)cc1S(=O)(=O)NCc1cccc(O)c1O] |
| | 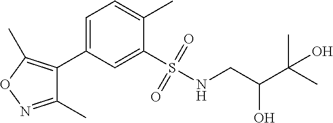 [SMILES: Cc1ccc(-c2c(C)noc2C)cc1S(=O)(=O)NCC(O)C(C)(C)O] |
| | 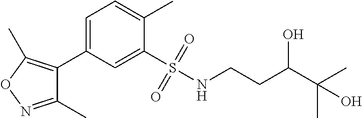 [SMILES: Cc1ccc(-c2c(C)noc2C)cc1S(=O)(=O)NCCC(O)C(C)(C)O] |
| | 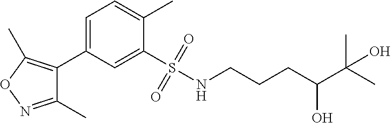 [SMILES: Cc1ccc(-c2c(C)noc2C)cc1S(=O)(=O)NCCCC(O)C(C)(C)O] |
| | 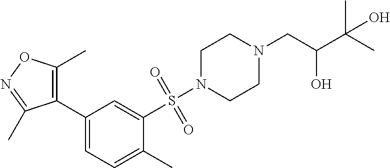 [SMILES: Cc1ccc(-c2c(C)noc2C)cc1S(=O)(=O)N1CCN(CC(O)C(C)(C)O)CC1] |

### Example 353

Table ZZ recites synthetic intermediates and comparative control compounds.

TABLE ZZ

| No. | Compound Structure |
|---|---|
| BRD-C-19 | 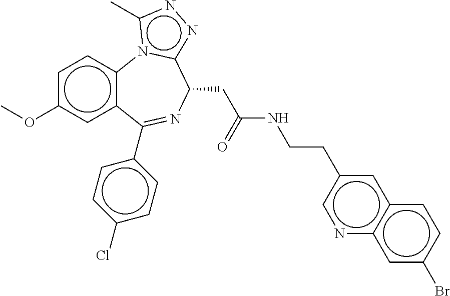 |
| BRD-C-17 | 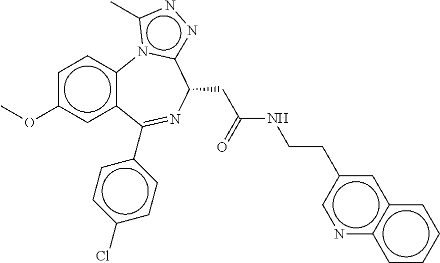 |
| BRD-C-15 | 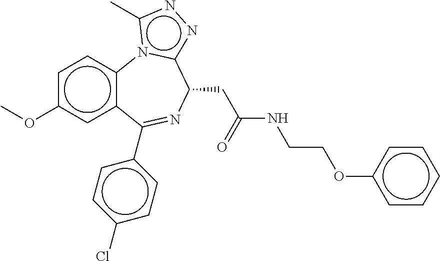 |
| BRD-C-16 | 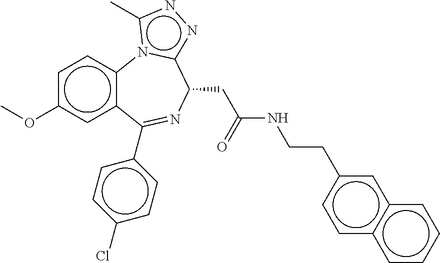 |
| BRD-C-14 | 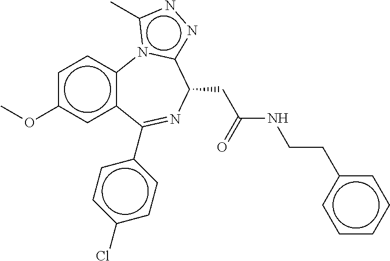 |
| BRD-C-13 | 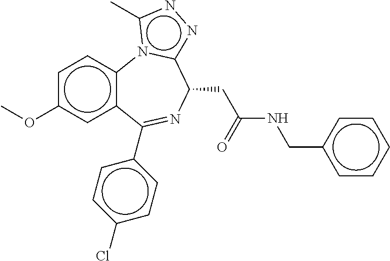 |
| BRD-C-01 | 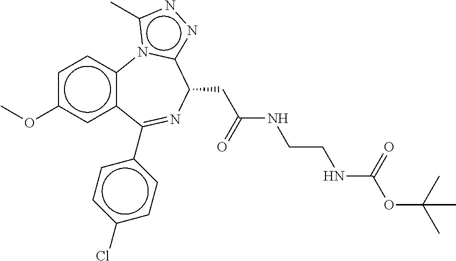 |
| BRD-C-02 | 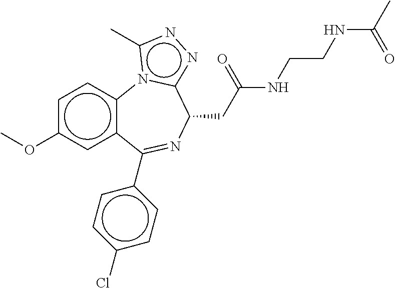 |
| BRD-C-03 | 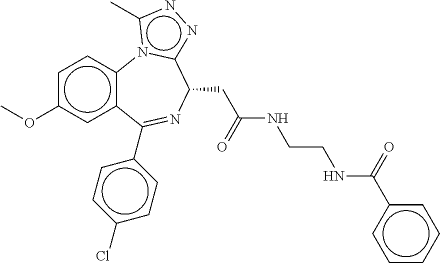 |
| BRD-C-04 | 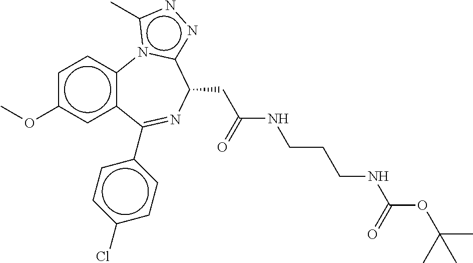 |
| BRD-C-05 | 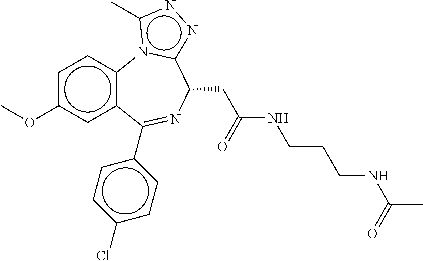 |
| BRD-C-06 | 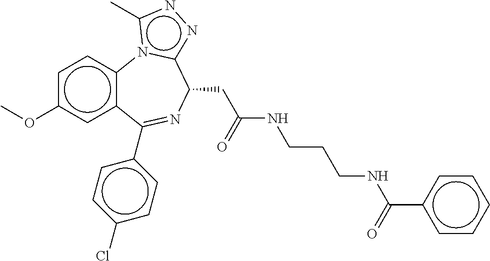 |
| BRD-C-07 | 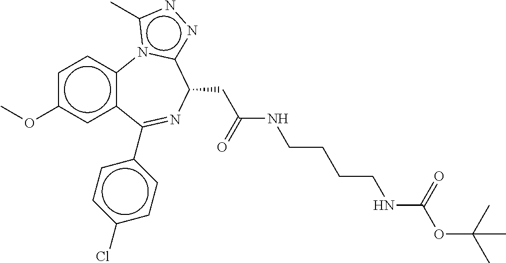 |
| BRD-C-08 | 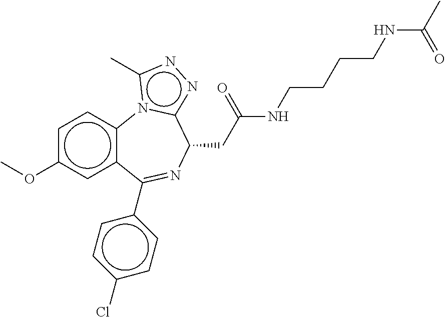 |
| BRD-C-09 | 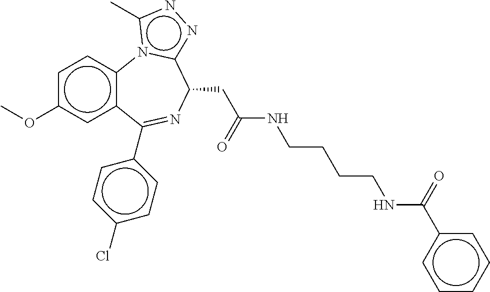 |
| BRD-C-10 | 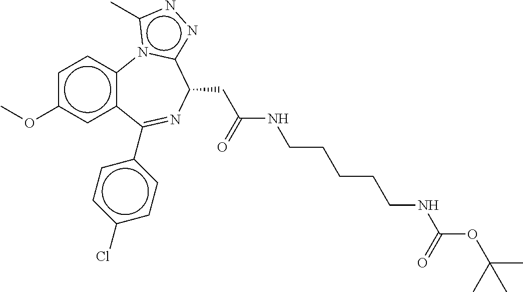 |
| BRD-C-11 | 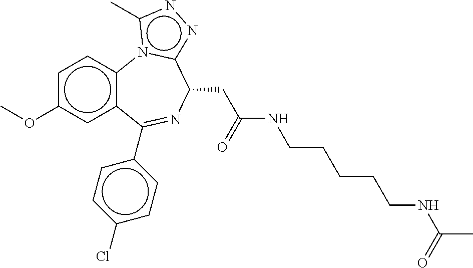 |
| BRD-C-12 | 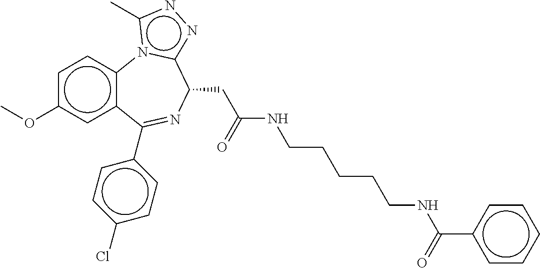 |
| BRD-C-34 | 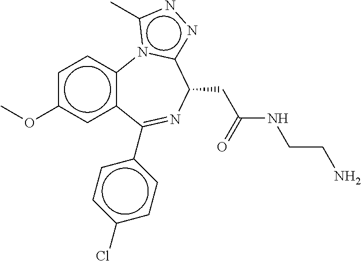 |
| BRD-C-35 | 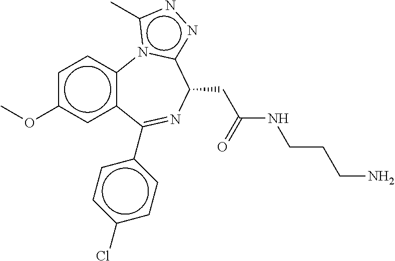 |
| BRD-C-36 | 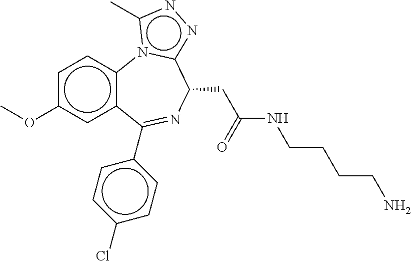 |
| BRD-C-37 | 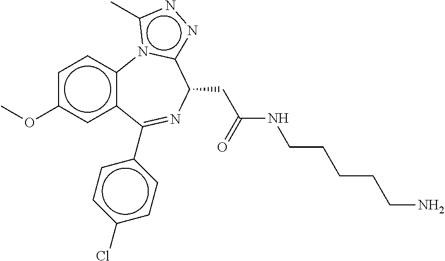 |
| BRD-C-20 | 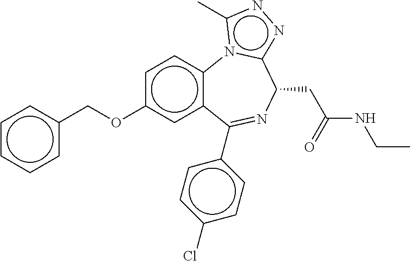 |
| BRD-C-21 | 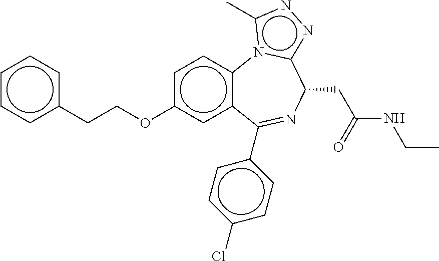 |
| BRD-C-22 | 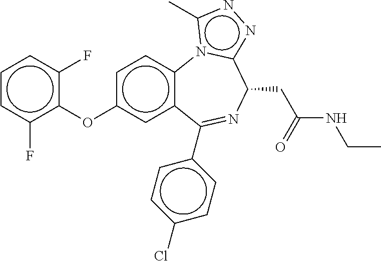 |
| BRD-C-23 | 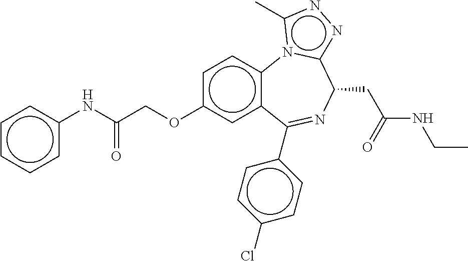 |
| BRD-C-24 | 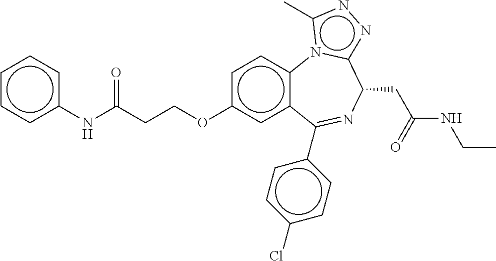 |
| BRD-C-25 | 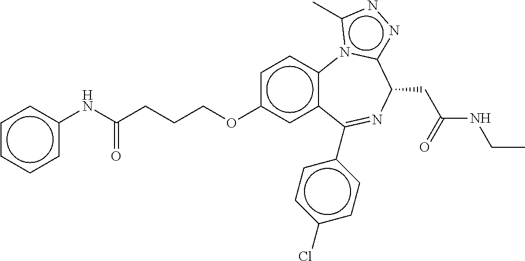 |
| BRD-C-26 | 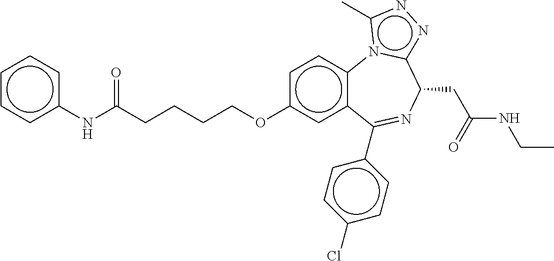 |
| BRD-C-27 Eq. C-41 | 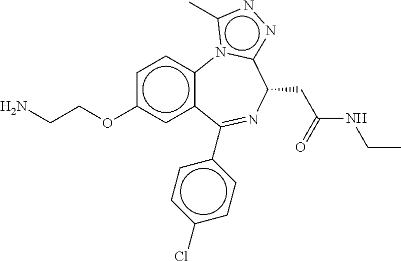 |
| BRD-C-28 | 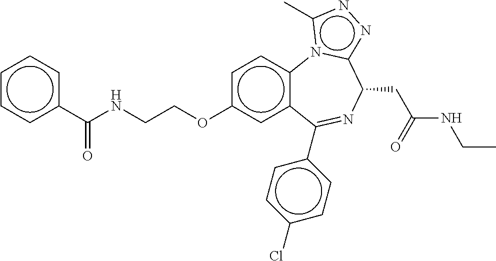 |
| BRD-C-29 | 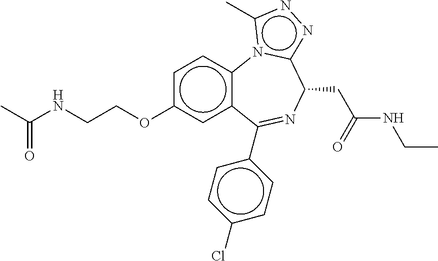 |
| BRD-C-30 eq. C-42 | 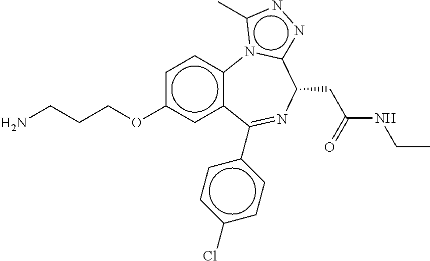 |
| BRD-C-31 | 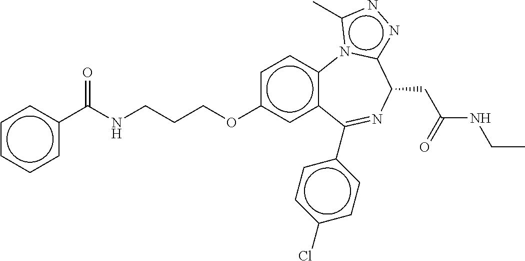 |
| BRD-C-32 | 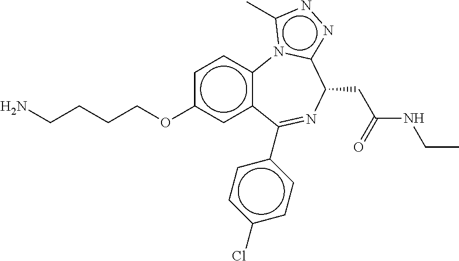 |
| BRD-C-33 | 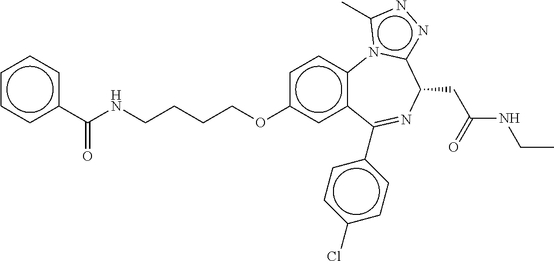 |
| BRD-C-43 | 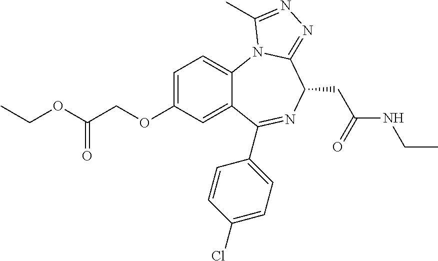 [SMILES: CCNC(=O)C[C@@H]1N=C(c2ccc(Cl)cc2)c2cc(OCC(=O)OCC)ccc2-n2c(C)nnc21] |
| BRD-C-44 | 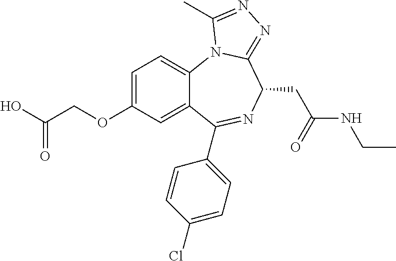 [SMILES: CCNC(=O)C[C@@H]1N=C(c2ccc(Cl)cc2)c2cc(OCC(=O)O)ccc2-n2c(C)nnc21] |
| BRD-C-45 | 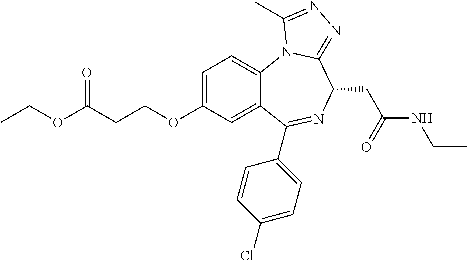 [SMILES: CCNC(=O)C[C@@H]1N=C(c2ccc(Cl)cc2)c2cc(OCCC(=O)OCC)ccc2-n2c(C)nnc21] |
| BRD-C-46 | 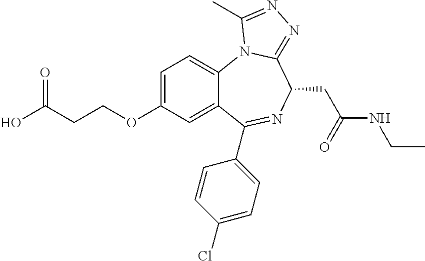 [SMILES: CCNC(=O)C[C@@H]1N=C(c2ccc(Cl)cc2)c2cc(OCCC(=O)O)ccc2-n2c(C)nnc21] |
| BRD-C-47 | 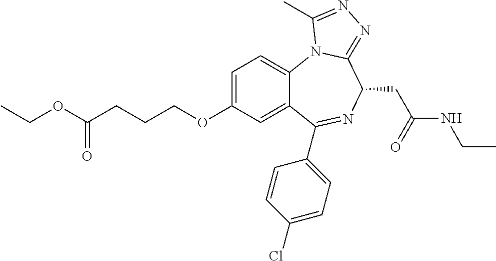 [SMILES: CCNC(=O)C[C@@H]1N=C(c2ccc(Cl)cc2)c2cc(OCCCC(=O)OCC)ccc2-n2c(C)nnc21] |
| BRD-C-48 | 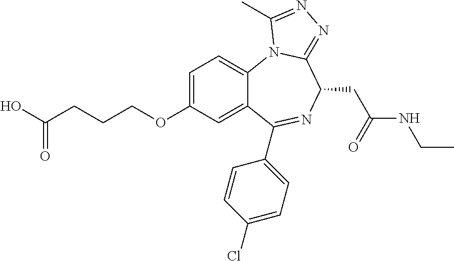 [SMILES: CCNC(=O)C[C@@H]1N=C(c2ccc(Cl)cc2)c2cc(OCCCC(=O)O)ccc2-n2c(C)nnc21] |
| BRD-C-49 | 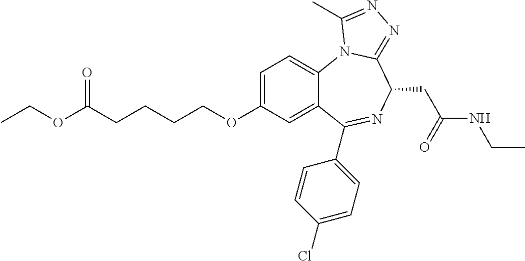 [SMILES: CCNC(=O)C[C@@H]1N=C(c2ccc(Cl)cc2)c2cc(OCCCCC(=O)OCC)ccc2-n2c(C)nnc21] |
| BRD-C-50 | 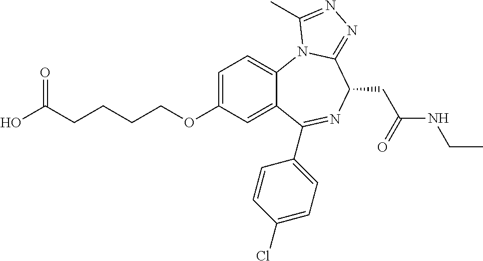 [SMILES: CCNC(=O)C[C@@H]1N=C(c2ccc(Cl)cc2)c2cc(OCCCCC(=O)O)ccc2-n2c(C)nnc21] |
| BRD-C-51 | 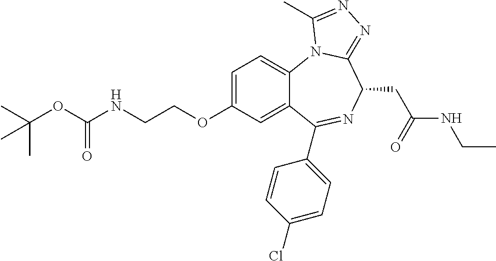 [SMILES: CCNC(=O)C[C@@H]1N=C(c2ccc(Cl)cc2)c2cc(OCCNC(=O)OC(C)(C)C)ccc2-n2c(C)nnc21] |
| BRD-C-52 | 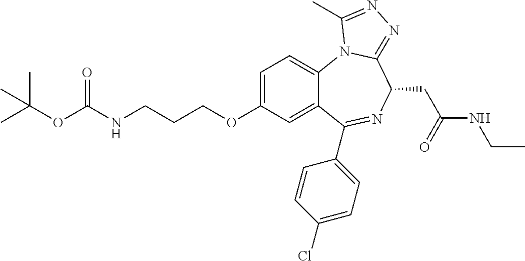 [SMILES: CCNC(=O)C[C@@H]1N=C(c2ccc(Cl)cc2)c2cc(OCCCNC(=O)OC(C)(C)C)ccc2-n2c(C)nnc21] |
| BRD-C-53 | 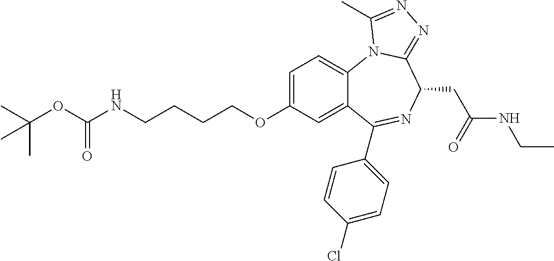 [SMILES: CCNC(=O)C[C@@H]1N=C(c2ccc(Cl)cc2)c2cc(OCCCCNC(=O)OC(C)(C)C)ccc2-n2c(C)nnc21] |
| BRD-C-54 | 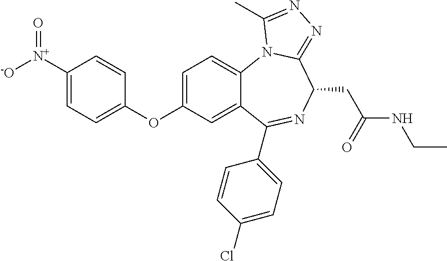 |
| BRD-C-55 | 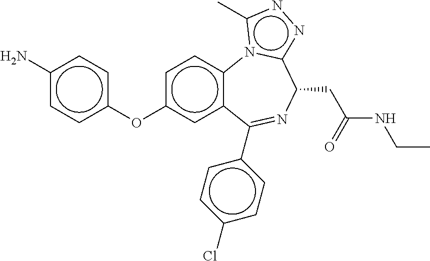 |
| BRD-C-56 | 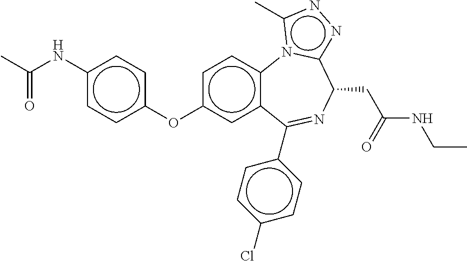 |
| BRD-C-57 | 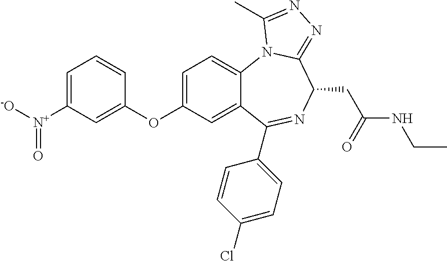 |
| BRD-C-58 | 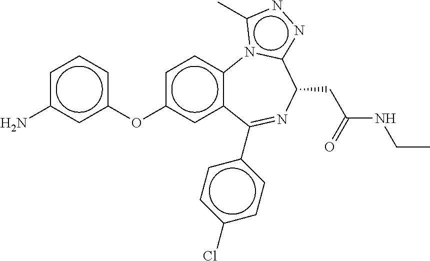 |
| BRD-C-59 | 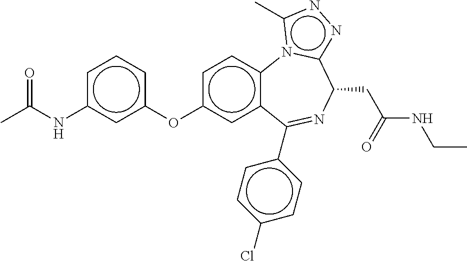 |
| BRD-C-60 | 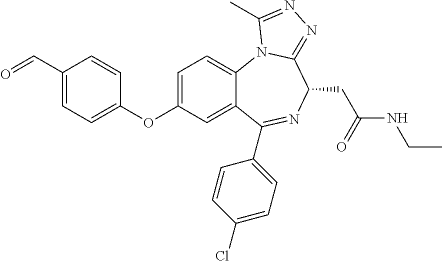 [SMILES: CCNC(=O)C[C@@H]1N=C(c2ccc(Cl)cc2)c2cc(Oc3ccc(C=O)cc3)ccc2-n2c(C)nnc21] |
| BRD-C-61 | 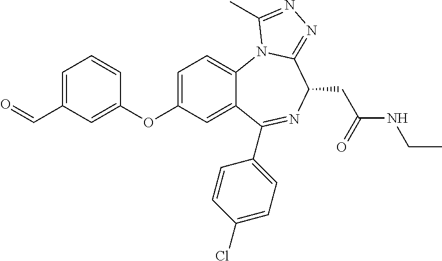 [SMILES: CCNC(=O)C[C@@H]1N=C(c2ccc(Cl)cc2)c2cc(Oc3cccc(C=O)c3)ccc2-n2c(C)nnc21] |
| BRD-C-62 | 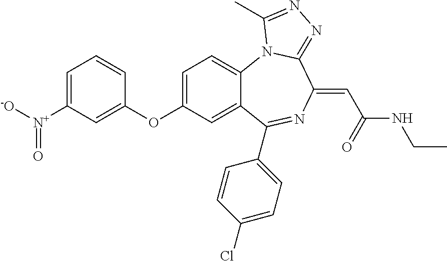 |
| BRD-C-63 | 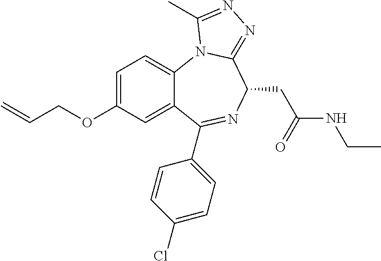 [SMILES: C=CCOc1ccc2c(c1)C(c1ccc(Cl)cc1)=N[C@@H](CC(=O)NCC)c1nnc(C)n1-2] |
| BRD-C-64 | 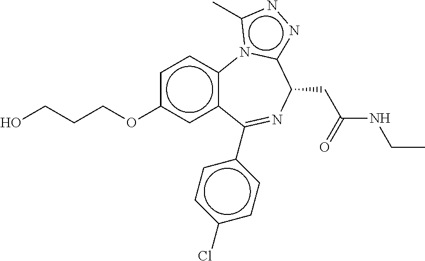 |
| BRD-C-65 | 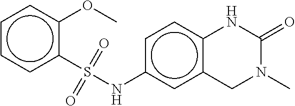 |
| BRD-C-66 | 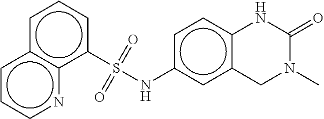 |
| BRD-C-67 | 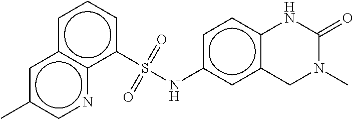 |
| BRD-C-68 | 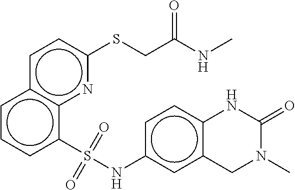 |
| BRD-C-69 | 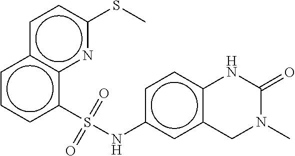 |
| BRD-C-70 | 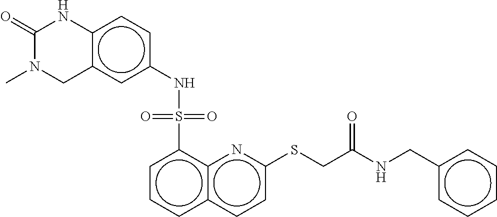 |
| BRD-C-71 | 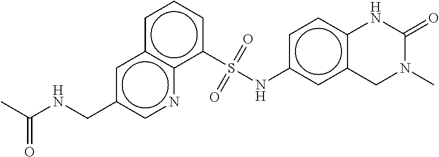 |
| BRD-C-72 | 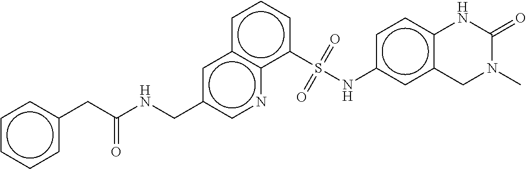 |
| BRD-C-73 | 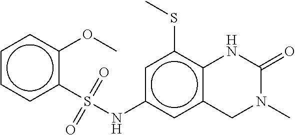 |
| BRD-C-74 | 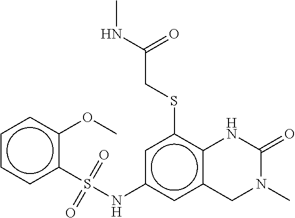 |
| BRD-C-75 | 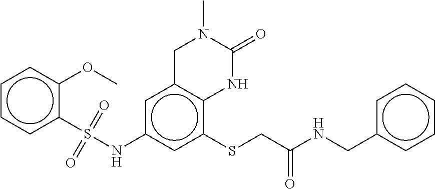 |
| BRD-C-76 | 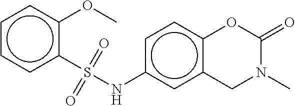 |
| BRD-C-77 | 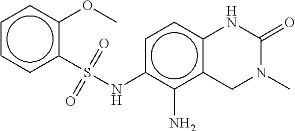 |
| BRD-C-78 | 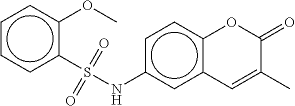 |
| BRD-C-79 | 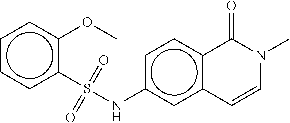 |
| BRD-C-80 | 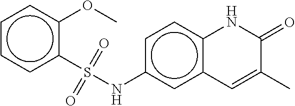 |
| BRD-C-81 | 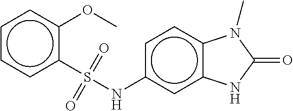 |

### Example 354

Monomers were synthesized according to the procedures described below.

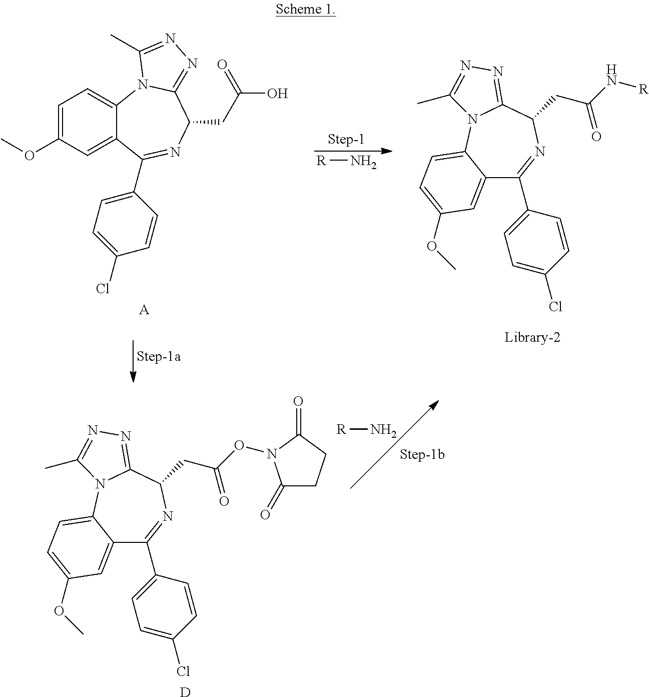

### Synthesis of (S)-(4-((2-(6-(4-chlorophenyl)-8-methoxy-1-methyl-4H-benzo[f][1,2,4]triazolo[4,3-a][1,4]diazepin-4-yl)acetamido)methyl)phenyl)boronic acid (BRD-E-09)

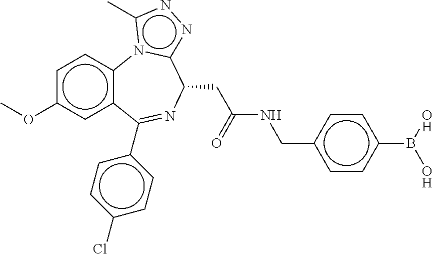

A solution of (S)-2-(6-(4-chlorophenyl)-8-methoxy-1-methyl-4H-benzo[f][1,2,4]triazolo[4,3-a][1,4]diazepin-4-yl)acetic acid (50 mg, 0.12 mmol) in DCM (10 mL) was charged with EDCI (36 mg, 0.18 mmol), DMAP (30.8 mg, 0.25 mmol), HOBt (25.5 mg, 0.17 mmol) and stirred at rt for 10 minutes. This solution was charged with (4-(aminomethyl)phenyl)boronic acid hydrochloride (23.6 mg, 0.12 mmol) and the resulting solution was stirred at room temperature overnight. The reaction mixture was partitioned between DCM and H₂O and separated. The aqueous layer was re-extracted with DCM (3 x 10 mL) and the combined organic fractions were dried over anhydrous Na₂SO₄, filtered and concentrated in vacuo resulting in a crude product which was purified by preparative HPLC to afford 30 mg, 45% yield of the corresponding title compound as a white solid. ¹H NMR (400 MHz, Methanol-d₄) δ 8.91-8.83 (m, 1H), 7.73 (dd, J=17.1, 8.5 Hz, 3H), 7.62 (d, J=7.7 Hz, 3H), 7.43-7.31 (m, 2H), 6.88 (d, J=3.0 Hz, 2H), 4.82 (s, 3H), 4.68-4.56 (m, 1H), 4.32 (dd, J=11.3, 7.2 Hz, 2H), 3.82 (s, 3H), 3.49 (dd, J=14.7, 9.9 Hz, 2H), 2.63 (s, 3H), 1.99 (s, 2H); Mol. Wt: 529.78; MS (ES+): m/z 530.15 [MH⁺], HPLC purity: 90.23% at Max plot.

Following the general procedure for synthesis of BRD-E-09, all below compounds have been synthesized and characterized.

### Synthesis of (S)-(3-((2-(6-(4-chlorophenyl)-8-methoxy-1-methyl-4H-benzo[f][1,2,4]triazolo[4,3-a][1,4]diazepin-4-yl)acetamido)methyl)phenyl)boronic acid (BRD-E-10)

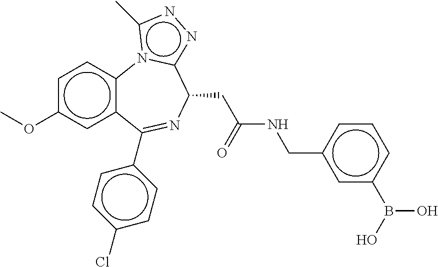

A solution of (S)-2-(6-(4-chlorophenyl)-8-methoxy-1-methyl-4H-benzo[f][1,2,4]triazolo[4,3-a][1,4]diazepin-4-yl)acetic acid (50 mg, 0.12 mmol) in DCM (10 mL) was charged with EDCI (36 mg, 0.18 mmol), DMAP (30.8 mg, 0.25 mmol), HOBt (25.5 mg, 0.17 mmol) and stirred at rt for 10 minutes. This solution was charged with (3-(aminomethyl)phenyl) boronic acid hydrochloride (23.6 mg, 0.12 mmol) resulting in 16 mg, 24% yield of the title compound as a white solid. ¹H NMR (400 MHz, Methanol-d₄) δ 7.71 (d, J=9.1 Hz, 2H), 7.61 (s, 1H), 7.53 (d, J=7.4 Hz, 1H), 7.45-7.31 (m, 6H), 6.90 (d, J=3.0 Hz, 1H), 5.49 (s, 1H), 4.68-4.54 (m, 3H), 4.33 (d, J=14.4 Hz, 1H), 3.82 (s, 3H), 3.48 (dd, J=14.6, 9.7 Hz, 1H), 3.24 (dd, J=15.1, 4.9 Hz, 2H), 2.63 (s, 3H), 1.29 (s, 1H), 0.10 (s, 1H). Mol. Wt: 529.78; MS (ES+): m/z 530.15 [MH⁺], HPLC purity: 99.90% at Max plot.

### Synthesis of (S)-(4-(2-(2-(6-(4-chlorophenyl)-8-methoxy-1-methyl-4H-benzo[f][1,2,4]triazolo[4,3-a][1,4]diazepin-4-yl)acetamido)ethyl)phenyl)boronic acid (BRD-E-13)

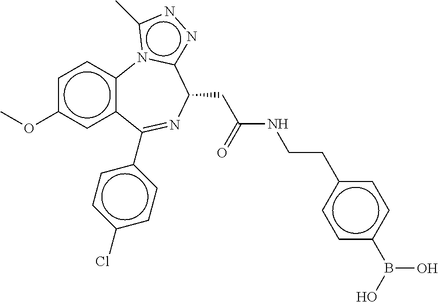

A solution of (S)-2-(6-(4-chlorophenyl)-8-methoxy-1-methyl-4H-benzo[f][1,2,4]triazolo[4,3-a][1,4]diazepin-4-yl)acetic acid (75 mg, 0.18 mmol) in DCM (10 mL) was charged with HATU (107 mg, 0.28 mmol), DIPEA (73 mg, 0.56 mmol), and stirred at rt for 10 minutes. This solution was charged with, (4-(2-aminoethyl)phenyl)boronic acid (45 mg, 0.18 mmol) resulting in 10 mg, 12% yield of the title compound as a white solid. ¹H NMR (400 MHz, Methanol-d₄) δ 7.75 (d, J=7.0 Hz, 1H), 7.65 (d, J=7.6 Hz, 1H), 7.45-7.55 (m, 7H), 7.40 (dd, J=8.0, 2.7 Hz, 1H), 7.25 (d, J=7.6 Hz, 1H), 4.63-4.75 (m, 1H), 3.85 (s, 3H), 3.20-3.65 (m, 4H), 2.88 (t, J=7.0 Hz, 2H), 2.71 (s, 3H), 1.34 (s, 2H). Mol. Wt: 543.81-boronic acid & 625.95-boronate ester; MS (ES+): m/z 543.80 [MH⁺] (boronic acid) 626.20 [MH⁺] (boronate ester), HPLC purity: 18.24% & 74.99%(mixture of boronic acid & ester) at Max plot.

### Synthesis of (S)-(3-(2-(2-(6-(4-chlorophenyl)-8-methoxy-1-methyl-4H-benzo[f][1,2,4]triazolo[4,3-a][1,4]diazepin-4-yl)acetamido)ethyl)phenyl)boronic acid (BRD-E-14)

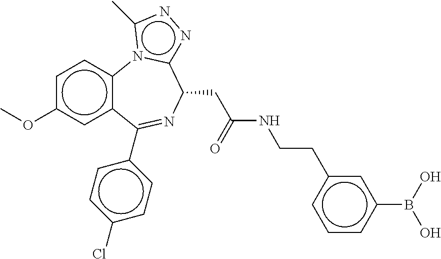

A solution of (S)-2-(6-(4-chlorophenyl)-8-methoxy-1-methyl-4H-benzo[f][1,2,4]triazolo[4,3-a][1,4]diazepin-4-yl)acetic acid (75 mg, 0.18 mmol) in DCM (10 mL) was charged with HATU (107 mg, 0.28 mmol), DIPEA (73 mg, 0.56 mmol), and stirred at rt for 10 minutes. This solution was charged with (3-(2-aminoethyl)phenyl)boronic acid (45 mg, 0.18 mmol) resulting in 36 mg, 30% yield of the title compound as a white solid. ¹H NMR (400 MHz, Methanol-d₄) δ 7.76 (d, J=9.0 Hz, 1H), 7.66 (s, 1H), 7.59 (d, J=7.4 Hz, 1H), 7.57-7.48 (m, 2H), 7.45-7.23 (m, 5H), 6.95 (d, J=3.0 Hz, 1H), 4.67 (dd, J=8.8, 5.3 Hz, 1H), 3.84 (s, 3H), 3.57-3.34 (m, 3H), 3.32-3.21 (m, 2H), 2.87 (t, J=7.2 Hz, 2H), 2.73 (s, 3H), 1.33 (s, 2H). Mol. Wt: 543.81-boronic acid & 625.95-boronate ester; MS (ES+): m/z—566.10 [M+Na] (boronic acid) 647.75 [M+Na] (boronate ester); HPLC purity: 21.4% & 71.3%(mixture of boronic acid & ester) at 220 nm.

### Synthesis of (S)-2-(6-(4-chlorophenyl)-8-methoxy-1-methyl-4H-benzo[f][1,2,4]triazolo[4,3-a][1,4]diazepin-4-yl)-N-(3,4-dihydroxybenzyl)acetamide (BRD-N-09)

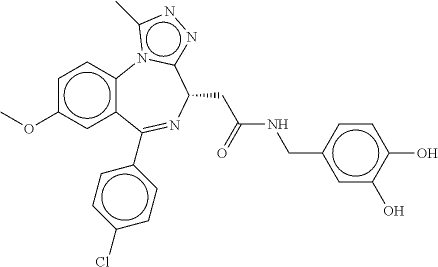

A solution of (S)-2-(6-(4-chlorophenyl)-8-methoxy-1-methyl-4H-benzo[f][1,2,4]triazolo[4,3-a][1,4]diazepin-4-yl)acetic acid (100 mg, 0.25 mmol) in DCM (10 mL) was charged with HATU (142 mg, 0.37 mmol), DIPEA (97 mg, 0.75 mmol), and stirred at rt for 10 minutes. This solution was charged with, 4-(aminomethyl)benzene-1,2-diol (55 mg, 0.25 mmol) resulting in 50 mg, 38% yield of the title compound as off white solid. ¹H NMR (400 MHz, Methanol-d₄) δ 7.75 (d, J=8.9 Hz, 1H), 7.39 (q, J=5.3, 3.1 Hz, 5H), 6.93 (d, J=3.0 Hz, 1H), 6.83-6.65 (m, 3H), 4.85 (d, J=8.8 Hz, 1H), 4.68 (dd, J=9.5, 4.6 Hz, 1H), 4.49-4.40 (m, 1H), 4.13 (d, J=14.6 Hz, 1H), 3.83 (s, 3H), 3.52-3.39 (m, 1H), 2.72 (s, 3H); Mol. Wt: 517.96, MS (ES+): m/z 518.10 [MH⁺], HPLC purity: 95.28% at Max plot.

### Synthesis of (S)-2-(6-(4-chlorophenyl)-8-methoxy-1-methyl-4H-benzo[f][1,2,4]triazolo[4,3-a][1,4]diazepin-4-yl)-N-(2,3-dihydroxybenzyl)acetamide (BRD-N-11)

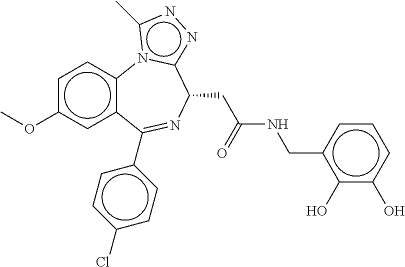

A solution of (S)-2-(6-(4-chlorophenyl)-8-methoxy-1-methyl-4H-benzo[f][1,2,4]triazolo[4,3-a][1,4]diazepin-4-yl)acetic acid (100 mg, 0.25 mmol) in DCM (10 mL) was charged with HATU (142 mg, 0.37 mmol), DIPEA (97 mg, 0.75 mmol), and stirred at rt for 10 minutes. This solution was charged with 3-(aminomethyl)benzene-1,2-diol (55 mg, 0.25 mmol) resulting in 40 mg, 30% yield of the title compound as off white solid. ¹H NMR (400 MHz, Methanol-d₄) δ 7.74 (d, J=9.3 Hz, 1H), 7.44-7.30 (m, 5H), 6.94-6.88 (m, 1H), 6.81-6.64 (m, 3H), 4.82 (d, J=11.7 Hz, 1H), 4.71-4.62 (m, 1H), 4.56 (dd, J=14.7, 3.1 Hz, 1H), 4.30-4.21 (m, 1H), 3.83 (s, 3H), 3.32-3.17 (m, 2H), 3.2 (t, 2H), 2.91 (t, 2H), 2.71 (s, 3H); Mol. Wt: 517.96; MS (ES+): m/z 540.05 [M+Na], HPLC purity: 96.22% at Max plot.

### Synthesis of (S)-2-(6-(4-chlorophenyl)-8-methoxy-1-methyl-4H-benzo[f][1,2,4]triazolo[4,3-a][1,4]diazepin-4-yl)-N-(2,3-dihydroxyphenethyl)acetamide (BRD-N-12)

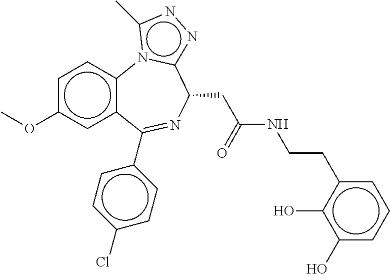

A solution of (S)-2-(6-(4-chlorophenyl)-8-methoxy-1-methyl-4H-benzo[f][1,2,4]triazolo[4,3-a][1,4]diazepin-4-yl)acetic acid (50 mg, 0.12 mmol) in DCM (10 mL) was charged with HATU (72 mg, 0.18 mmol) and DIPEA (49 mg, 0.37 mmol) and stirred at rt for 10 minutes. This solution was charged with 3-(aminoethyl)benzene-1,2-diol (30 mg, 0.12 mmol) resulting in 8 mg, 12% yield of the title compound as off white solid. ¹H NMR (400 MHz, Methanol-d₄) δ 7.74 (d, J=9.3 Hz, 1H), 7.44-7.30 (m, 5H), 6.94-6.88 (m, 1H), 6.81-6.64 (m, 3H), 4.82 (d, J=11.7 Hz, 1H), 4.71-4.62 (m, 1H), 4.56 (dd, J=14.7, 3.1 Hz, 1H), 4.30-4.21 (m, 1H), 3.83 (s, 3H), 3.32-3.17 (m, 1H), 2.71 (s, 3H); Mol. Wt: 531.99; MS (ES+): m/z 532.10 [MH⁺], HPLC purity: 96.28% at Max plot

### Synthesis of (S)-2-(6-(4-chlorophenyl)-8-methoxy-1-methyl-4H-benzo[f][1,2,4]triazolo[4,3-a][1,4]diazepin-4-yl)-N-(2-(4-(hydroxydimethylsilyl)phenoxy)ethyl)acetamide (BRD-S-09)

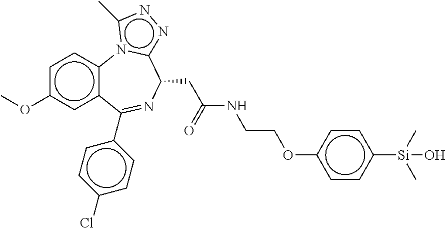

A solution of (S)-2-(6-(4-chlorophenyl)-8-methoxy-1-methyl-4H-benzo[f][1,2,4]triazolo[4,3-a][1,4]diazepin-4-yl)acetic acid (50 mg, 0.12 mmol) in DCM (10 mL) was charged with HATU (72 mg, 0.18 mmol) and DIPEA (49 mg, 0.37 mmol) and stirred at rt for 10 minutes. This solution was charged with (4-(2-aminoethoxy)phenyl)dimethylsilanol (26.6 mg, 0.12 mmol) resulting in 4 mg, 5.4% yield of the title compound as off white solid. ¹H NMR (400 MHz, Methanol-d₄) δ 7.72 (dd, J=9.1, 4.4 Hz, 1H), 7.42 (dd, J=24.8, 8.8 Hz, 5H), 7.13 (t, J=7.8 Hz, 2H), 6.90 (dd, J=8.3, 4.3 Hz, 3H), 4.84 (d, J=11.7 Hz, 1H), 4.68 (dd, J=9.3, 4.7 Hz, 1H), 4.13-4.03 (m, 1H), 3.85-3.71 (m, 2H), 3.50-3.15 (m, 2H), 2.71 (s, Hz, 3H), 0.38-0.27 (s, 6H); Mol. Wt: 590.14 (monomer), 1162.27 (dimer); MS (ES+): m/z 590.11 [MH⁺], 581.90[MH+/2]-dimer, HPLC purity:—8.2% for monomer & 86.79% for dimer at Max plot

### Synthesis of (S)-2-(6-(4-chlorophenyl)-8-methoxy-1-methyl-4H-benzo[f][1,2,4]triazolo[4,3-a][1,4]diazepin-4-yl)-N-(2-(3-(hydroxydimethylsilyl)phenoxy)ethyl)acetamide (BRD-S-10)

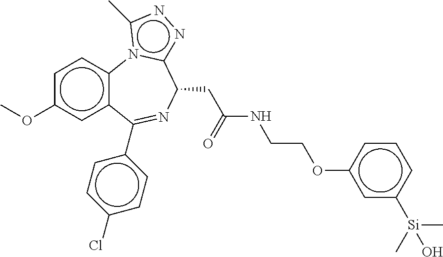

A solution of (S)-2-(6-(4-chlorophenyl)-8-methoxy-1-methyl-4H-benzo[f][1,2,4]triazolo[4,3-a][1,4]diazepin-4-yl)acetic acid (40 mg, 0.10 mmol) in DCM (10 mL) was charged with EDCI (28.9 mg, 0.15 mmol), DMAP (18 mg, 0.15 mmol), and HOBt (20.4 mg, 0.15 mmol) and stirred at rt for 10 minutes. This solution was charged with (3-(2-aminoethoxy)phenyl)dimethylsilanol (21 mg, 0.10 mmol) resulting in 4 mg, 5.4% yield of the title compound as off white solid. ¹H NMR (400 MHz, Methanol-d₄) δ 7.71 (d, J=8.7 Hz, 1H), 7.48-7.27 (m, 4H), 7.23-7.03 (m, 3H), 6.98 (dd, J=8.2, 2.8 Hz, 1H), 6.91-6.82 (m, 1H), 4.63 (dd, J=9.8, 4.7 Hz, 1H), 4.12 (dd, J=8.1, 3.3 Hz, 1H), 3.80 (s, 3H), 3.61-3.39 (m, 2H), 3.28-3.15 (m, 3H), 2.62 (s, 3H), 0.37-0.26 (s, 6H); Mol. Wt: 590.14 (monomer), 1162.27 (dimer); MS (ES+): m/z 590.20 [MH⁺], 582.5[M/2+1]-dimer, HPLC purity: 81.8% for monomer & 17.66% for dimer at Max plot.

### Synthesis of (S)-2-(6-(4-chlorophenyl)-8-methoxy-1-methyl-4H-benzo[f][1,2,4]triazolo[4,3-a][1,4]diazepin-4-yl)-N-(3-(hydroxydimethylsilyl)phenethyl)acetamide (BRD-S-12)

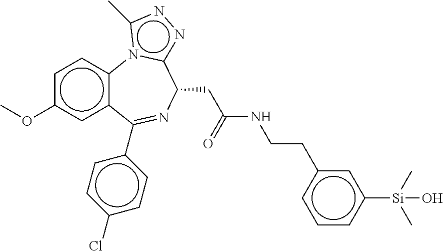

A solution of (S)-2-(6-(4-chlorophenyl)-8-methoxy-1-methyl-4H-benzo[f][1,2,4]triazolo[4,3-a][1,4]diazepin-4-yl)acetic acid (75 mg, 0.18 mmol) in DCM (10 mL) was charged with HATU (107 mg, 0.28 mmol) and DIPEA (73 mg, 0.50 mmol), and stirred at rt for 10 minutes. This solution was charged with (3-(2-aminoethyl)phenyl)dimethylsilanol (35 mg, 0.28 mmol) resulting in 13 mg, 13% yield of the title compound as off white solid. ¹H NMR (400 MHz, Methanol-d₄) δ 7.74 (d, J=8.9 Hz, 1H), 7.58-7.48 (m, 2H), 7.36-7.40 (m, 4H), 7.26 (dd, J=4.5, 2.2 Hz, 2H), 6.93 (d, J=2.9 Hz, 1H), 4.66 (dd, J=8.6, 5.6 Hz, 1H), 3.82 (s, 3H), 3.40 (dq, J=24.6, 8.7, 7.9 Hz, 3H), 3.25 (dd, J=15.1, 5.6 Hz, 1H), 2.82 (t, J=7.4 Hz, 2H), 2.72 (s, 3H), 1.29 (s, 1H), 0.36-0.25 (m, 6H); Mol. Wt: 574.15 (monomer), 1130.28 (dimer); MS (ES+): m/z 574.05 [MH⁺]-monomer, m/z 1153.15 [MH⁺]-dimer, HPLC purity: 14.2% for monomer & 81.01% for dimer at Max plot.

### Synthesis of (S)-2-(6-(4-chlorophenyl)-8-methoxy-1-methyl-4H-benzo[f][1,2,4]triazolo[4,3-a][1,4]diazepin-4-yl)-N-(3,4-dihydroxyphenethyl)acetamide (BRD-N-10)

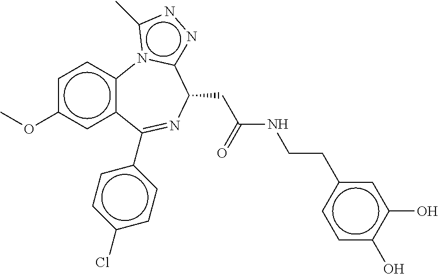

A solution of 4-(2-aminoethyl)benzene-1,2-diol hydrochloride (30 mg, 0.12 mmol) in THF (5 mL) was charged with TEA (10 mg, 0.20 mmol), and stirred at rt for 10 minutes. This solution was charged with (S)-2,5-dioxopyrrolidin-1-yl-2-(6-(4-chlorophenyl)-8-methoxy-1-methyl-4H-benzo[f][1,2,4]triazolo[4,3-a][1,4]diazepin-4-yl)acetate (50 mg, 0.12 mmol) and stirred at 50° C. for an additional 6 h. The reaction concentrated in vacuo resulting in a crude product which was purified by preparative HPLC to afford 12 mg, 22% yield of the corresponding title compound as a white solid. ¹H NMR (400 MHz, DMSO-d₆) δ 7.78 (d, J=9.1 Hz, 1H), 7.47 (s, 1H), 7.43-7.35 (m, 1H), 6.85 (s, 1H), 6.68-6.57 (m, 4H), 6.46 (d, J=8.0 Hz, 2H), 4.54-4.41 (m, 1H), 3.78 (s, 3H), 3.68 (d, J=8.4 Hz, 1H), 3.30-3.07 (m, 4H), 2.56 (s, 3H), 1.75 (d, J=6.5 Hz, 1H); Mol. Wt: 531.99; MS (ES+): m/z 532.15 [MH⁺], HPLC purity: 99.21% at Max plot.

### Intermediate Synthesis

### (4-(2-aminoethoxy)phenyl)dimethylsilanol

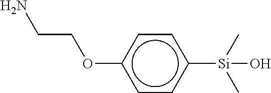

General Procedure for De-Protection of Phthalimide:

A solution of 2-(2-(4-(hydroxydimethylsilyl)phenoxy)ethyl)isoindoline-1,3-dione (350 mg, 1.0 mmol) in ethanol (10 ml) was charged with hydrazine hydrate(monohydrate) (256 mg, 5.1 mmol) and heated at 60° C. for 4 h. The reaction mixture was cooled to room temperature and filtered through a pad of celite. The resulting filtrate was concentrated in vacuo resulting in 180 mg, 83% yield of crude semi solid product which was used in the next step without further purification Mol. Wt: 211.33; MS (ES+): m/z 212.30 [MH⁺].

The following compounds were synthesized using the general procedure described above.

### 3-(2-aminoethoxy)phenyl)dimethylsilanol

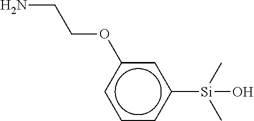

A solution of 2-(2-(3-(hydroxydimethylsilyl)phenoxy)ethyl)isoindoline-1,3-dione (140 mg, 0.4 mmol) in ethanol (5 ml) was charged with hydrazine hydrate(monohydrate) (102 mg, 2.0 mmol) and heated at 60° C. for 4 h resulting in 72 mg, yield: 83.7% as a semi solid; Mol. Wt: 211.33; MS (ES+): m/z 212.30 [MH⁺].

### (3-(2-aminoethyl)phenyl)dimethylsilanol

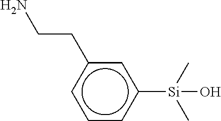

General Procedure for Boc-Deprotection:

A solution of tert-butyl 3-(hydroxydimethylsilyl)phenethylcarbamate (100 mg, 0.33 mmol) in DCM (5 mL) was charged with TFA (27 mg, 2.3 mmol) and stirred for at rt for 3 h. The reaction mixture was concentrated in vacuo to obtain a residue which was triturated with diethyl ether to afford 60 mg, 90% yield of the title compound as an oil, Mol. Wt: 195.33; MS (ES+): m/z 196.25 [MH⁺].

### 2-(2-(4-(hydroxydimethylsilyl)phenoxy)ethyl)isoindoline-1,3-dione

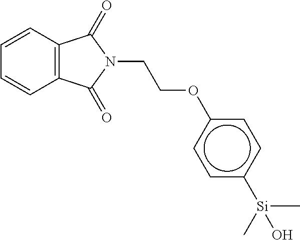

General Procedure for Silylation:

A solution of 2-(2-(4-bromophenoxy)ethyl)isoindoline-1,3-dione (1.00 g, 2.88 mmol) in NMP (10 ml) was charged with (2-biphenyl)ditert-butyl phosphine (171 mg, 0.57 mmol), palladium chloride (51 mg, 0.28 mmol) and DIPEA (2.97 g, 23 mmol) and stirred at room temperature for 20 min under argon atmosphere then charged with 1,2-diethoxy-1,1,2,2-tetramethyldisilane (4.74 g, 23 mmol) and heated at 60° C. for 14 h. The reaction mixture was cooled to room temperature, diluted with ethyl acetate (20 ml), and separated. The organic layer was washed with brine, dried over Na₂SO₄, and concentrated in vacuo resulting in a crude product which was purified by column chromatography on silica gel (230-400 mesh), eluting with 5% methanol in chloroform resulting in 150 mg of product. Mol. Wt: 341.43; MS (ES+): m/z 342.1 [MH⁺].

The following compounds were synthesized using the general procedure described above.

### 2-(2-(3-(hydroxydimethylsilyl)phenoxy)ethyl)isoindoline-1,3-dione

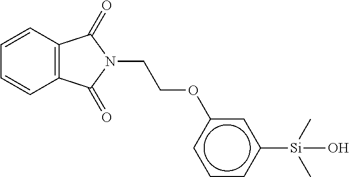

A solution of 2-(2-(3-bromophenoxy)ethyl)isoindoline-1,3-dione (500 mg, 1.45 mmol) in NMP (5 ml) was charged with (2-biphenyl)ditert-butyl phosphine (85.8 mg, 0.28 mmol), palladium chloride (25 mg, 0.14 mmol), DIPEA (1.1 g, 8.6 mmol) and the solution stirred at room temperature for 20 min under argon atmosphere. The reaction was then charged with 1, 2-diethoxy-1,1,2,2-tetramethyldisilane (1.78 g, 8.6 mmol) and heated at 60° C. for 14 h. Mol. Wt: 341.43; MS (ES+): m/z 342.1 [MH⁺].

### tert-butyl 3-(hydroxydimethylsilyl)phenethylcarbamate

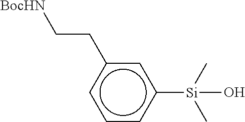

A solution of tert-butyl 3-bromophenethylcarbamate (1.00 g, 3.5 mmol) in NMP (5 ml) was charged with (2-biphenyl)ditert-butyl phosphine (209 mg, 0.7 mmol), palladium chloride (62 mg, 0.35 mmol), and DIPEA (2.5 g, 21 mmol) and stirred at room temperature for 20 min under argon atmosphere. The solution was charged with 1,2-diethoxy-1,1,2,2-tetramethyldisilane (2.1 g, 21 mmol) and heated at 60° C. for 14 h. Mol. Wt: 300.19; MS (ES+): m/z 301.1 [MH⁺].

### General Procedure for N-Alkylations

### 2-(2-(3-bromophenoxy)ethyl) isoindoline-1,3-dione

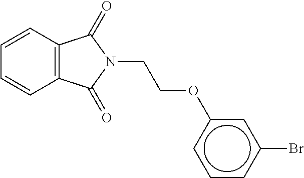

A solution of 1-bromo-4-(2-bromoethoxy)benzene (4.5 g, 16 mmol) in DMF (10 ml) was charged with potassium iodide (4 g, 21 mmol) and potassium phthalimide (2.17 g, 16 mmol) under inert atmosphere. The resulting solution was heated at 80° C. for 4 h and then allowed to cool to room temperature. The reaction mixture was partitioned between water (50 mL) and acetate (50 mL) and separated. The aqueous was re-extracted with ethyl acetate (2×50 mL) and the combined organic fractions were dried over anhydrous Na₂SO₄, filtered and concentrated in vacuo resulting in a crude product which was purified by column chromatography on silica gel (230-400 mesh), eluting with 2% methanol in chloroform to afford 4 g, 72% yield of the title compound as a white solid; Mol. Wt: 346.18; MS (ES+): m/z 347.20 [MH⁺].

The following compounds were synthesized using the general procedure described above.

### 2-(2-(4-bromophenoxy)ethyl)isoindoline-1,3-dione

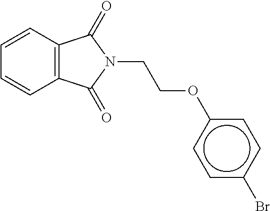

A solution of 1-bromo-3-(2-bromoethoxy)benzene (3 g, 10 mmol) in DMF (10 ml) was charged with potassium iodide (2.6 g, 16 mmol) and potassium phthalimide (1.98 g, 10 mmol) under inert atmosphere. And heated at 80° C. for 4 h resulting in 3.5 g, 94% yield of the title compound as a white solid after purification; Mol. Wt: 346.18; MS (ES+): m/z 347.20 [MH⁺].

### General Procedure for Boc-Protection

### tert-butyl 3-bromophenethylcarbamate

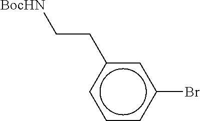

A solution of 2-(3-bromophenyl)ethanamine (2 g, 10 mmol) in DCM (50 ml) was cooled to 0° C. and charged with TEA (1.21 g, 12 mmol) and boc anhydride (2.39 g, 11 mmol) under inert atmosphere. The resulting solution was stirred at 0° C. for an additional 4 h. The reaction mixture was partitioned between water r (50 mL) and DCM (10 mL) and separated. The aqueous was re-extracted with DCM (2×10 mL). and the combined organic fractions were washed with 2N HCl, dried over anhydrous Na₂SO₄, filtered and concentrated in vacuo resulting in a crude product which was used in the next reaction without further purification. Mol. Wt: 300.19; MS (ES+): m/z 301.20 [MH⁺].

### General Procedure for O-Alkylation

### 1-bromo-4-(2-bromoethoxy)benzene

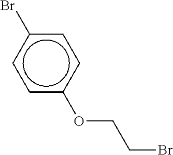

A solution of 4-bromophenol (3 g, 17 mmol) in acetone was charged with potassium carbonate (14.36 g mg, 104 mmol) and 1,2-dibromoethane (19.5 g, 104 mmol) under inert atmosphere. The resulting solution was heated at 80° C. for 14 h. The reaction mixture was cooled to room temperature, filtered, and the filtrate was concentrated in vacuo resulting in a 4.60 g, 94% yield of the crude product as a white solid. The material was used in the next step without further purification. ¹H NMR (400 MHz, DMSO-d₆) δ 7.42 (d, J=8.0 Hz, 2H), 7.00 (d, J=8.4 Hz, 2H), 4.4 (t, J=7.0 Hz, 2H), 3.9 (t, J=7.1 Hz, 2H).

The following compounds were synthesized using the general procedure described above.

### 1-bromo-3-(2-bromoethoxy)benzene

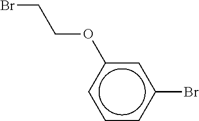

A solution of 4-bromophenol (0.5 g, 2.8 mmol) in acetone was charged with potassium carbonate (2.39 g mg, 1.7 mmol) and 1,2-dibromoethane (3.2 g, 1.7 mmol) under inert atmosphere. The resulting solution was heated at 80° C. for 14 h resulting in 800 mg; 99% yield of the title compound as a white solid. ¹H NMR (400 MHz, DMSO-d₆) δ 7.40 (t, J=8.2 Hz, 1H), 7.30 (d, J=8.6 Hz, 2H), 7.00 (s, 1H), 4.40 (t, J=6.3 Hz, 2H), 3.90 (t, J=6.5 Hz, 2H).

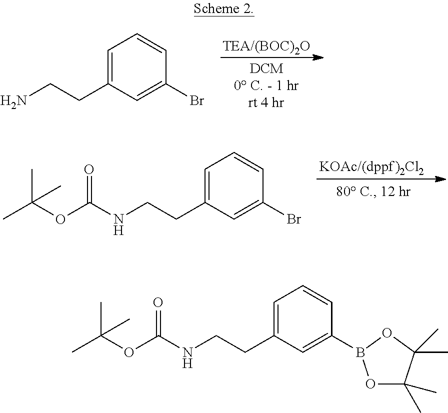 [SMILES: NCCc1cccc(Br)c1 ; CC(C)(C)OC(=O)NCCc1cccc(Br)c1 ; CC(C)(C)OC(=O)NCCc1cccc(B2OC(C)(C)C(C)(C)O2)c1]

tert-butyl 3-(4,4,5,5-tetramethyl-1,3,2-dioxaborolan-2-yl)phenethylcarbamate:

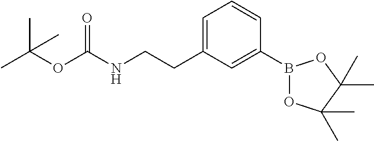 [SMILES: CC(C)(C)OC(=O)NCCc1cccc(B2OC(C)(C)C(C)(C)O2)c1]

A solution of tert-butyl 3-bromophenethylcarbamate (270 mg, 0.90 mmol) in DMSO (15 mL) was charged with potassium acetate (309 mg, 3.15 mmol), bis-pinacolatodiborane (1.14 gm, 0.045 mmol), and Pd(dppf), Cl₂ (44 mg, 0.54 mmol) under argon. The reaction mixture was heated at 80° C. for 12 h then allowed to cool to room temperature and partitioned between water (20 mL) and ethyl acetate (20 mL) and separated. The aqueous was re-extracted with ethyl acetate (2×20 mL) and the combined organic fractions were washed with brine, dried over anhydrous Na₂SO₄, filtered, and concentrated in vacuo resulting 450 mg of title compund as crude black color oil. Mol. Wt: 347.26, MS (ES+): m/z 370.25[M+Na].

### Step-1: tert-butyl 3-bromophenethylcarbamate

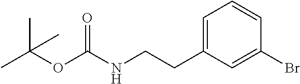 [SMILES: CC(C)(C)OC(=O)NCCc1cccc(Br)c1]

A solution of 3-bromophenylethylamine (200 mg, 1.00 mmol) in DCM (iOmL) was charged with triethylamine (0.12 gm, 1.20 mmol) was cooled to 0-5° C. then charged with boc-anhydride (240 mg, 1.10 mmol) and stirred at this temperature for 30 min. at 0-5° C. for 30 mins then allowed to reach room temperature and stirred for an additional 4 hr. The reaction mixture was partitioned with 1N KHSO₄ (10 mL) and the DCM (50 mL) and separated. The aqueous was re-extracted with DCM (2×50 mL) and the combined organic fractions were washed with brine (2 x 10 mL), dried over anhydrous Na₂SO₄, filtered and concentrated in vacuo resulting in 270 mg, 90% yield of the title compound as a colorless oil.

¹H NMR (400 MHz, CDCl₃) δ 7.40-7.32 (m, 2H), 7.22-7.09 (m, 2H), 3.36 (t, J=6.8 Hz, 2H), 2.77 (t, J=7.2 Hz, 2H), 1.55 (s, 9H).

### Intermediate Synthesis:

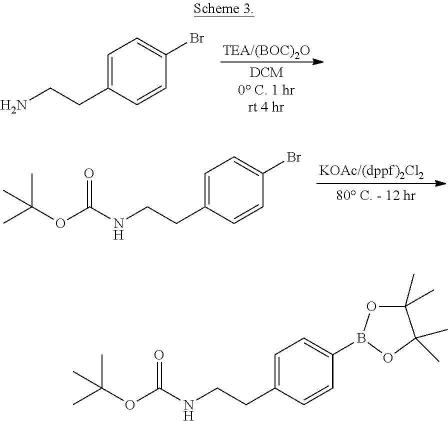 [SMILES: NCCc1ccc(Br)cc1 ; CC(C)(C)OC(=O)NCCc1ccc(Br)cc1 ; CC(C)(C)OC(=O)NCCc1ccc(B2OC(C)(C)C(C)(C)O2)cc1]

### tert-butyl 4-(4,4,5,5-tetramethyl-1,3,2-dioxaborolan-2-yl)phenethylcarbamate

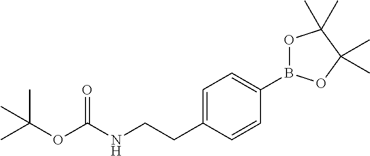 [SMILES: CC(C)(C)OC(=O)NCCc1ccc(B2OC(C)(C)C(C)(C)O2)cc1]

A solution of tert-butyl 4-bromophenethylcarbamate (270 mg, 0.9 mmol) in DMSO (15 mL) was charged with potassium acetate (309 mg, 3.15 mmol), bis-pinacolatodiborane (1.14 g, 0.45 mmol), and Pd(dppf), Cl₂ (44 mg, 0.54 mmol) under argon atmopshere and the reaction mixture was heated at 80° C. for 12 hr. The reaction mixture was allowed to cool to room temperature and partitioned between water (20 mL) and ethyl acetate (20 mL) and separated. The aqueous was re-extracted with ethyl acetate (2×20 mL) and the combined organic fractions were washed with brine (1 x 10 mL), dried over anhydrous Na₂SO₄, filtered and concentrated in vacuo resulting in 440 mg of the title compound as black color oil. Mol. Wt: 347.26, MS (ES+): m/z 370.20 [M+Na].

### Step-1: tert-butyl 4-bromophenethylcarbamate

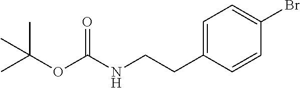 [SMILES: CC(C)(C)OC(=O)NCCc1ccc(Br)cc1]

A solution of 4-bromophenylethylamine (200 mg, 1.0 mmol) and triethylamine (0.12 g, 1.20 mmol) in DCM (10 mL) was cooled to 0-5° C. and charged with and boc-anhydride (0.12 g, 1.20 mmol) and stirred at this temperature for 30 min then allowed to reach room temperature and stirred for an additional 4 hr. The reaction mixture was partitioned between 1N KHSO₄ (10 mL) and the DCM (30 mL) and separated. The aqueous was re-extracted with DCM (2×30 mL) and the combined organic fraction were was washed with brine solution (2 x 10 mL), dried over anhydrous Na₂SO₄, filtered, and concentrated in vacuo resulting in 280 mg, 93.33% yield of the title compound as a colorless oil. ¹H NMR (400 MHz, CDCl₃) δ 7.46-7.39 (m, 2H), 7.07 (d, J=8.0 Hz, 2H), 3.34 (t, J=6.9 Hz, 2H), 2.75 (t, J=7.1 Hz, 2H), 1.57 (s, 9H).

### Example 355

Monomers were synthesized according to the procedures described below.

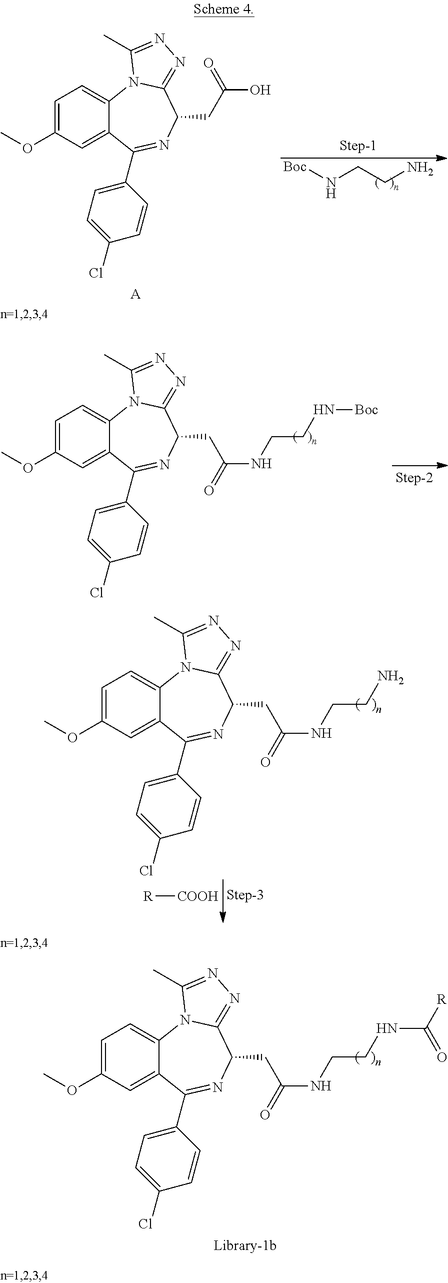

### Synthesis of (4-(2-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-2-oxoethyl)phenyl)boronic acid (BRD-E-27)

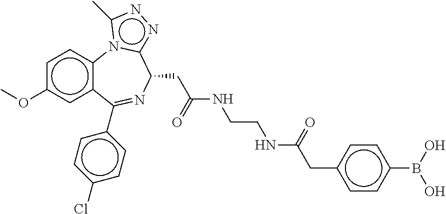

A solution of 2-(4-(4,4,5,5-tetramethyl-1,3,2-dioxaborolan-2-yl)phenyl)acetic acid (29.8 mg, 0.11 mmol) in DCM (10 mL) was charged with EDCI (32 mg, 0.17 mmol), DMAP (16 mg, 0.13 mmol), and HOBt (23 mg, 0.17 mmol) and stirred at rt for 10 minutes. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and stirred at room temperature overnight. The reaction mixture was partitioned between DCM and H₂O and separated. The aqueous layer was re-extracted with DCM (3×10 mL) and the combined organic fractions were dried over anhydrous Na₂SO₄, filtered, and concentrated in vacuo resulting in a crude product which was purified by preparative HPLC to afford 49 mg, 63% yield of the title compound as a white solid of a mixture of boronic acid and ester. ¹H NMR (400 MHz, Methanol-d₄) δ 7.67 (dt, J=20.8, 6.1 Hz, 4H), 7.57-7.45 (m, 2H), 7.38 (t, J=8.4 Hz, 2H), 7.26 (d, J=7.5 Hz, 2H), 6.92 (t, J=3.5 Hz, 1H), 4.82 (d, J=12.3 Hz, 1H), 4.60 (p, J=5.5 Hz, 1H), 3.81 (s, 3H), 3.52 (d, J=3.7 Hz, 1H), 3.45-3.32 (m, 4H), 3.25 (d, J=18.2 Hz, 2H), 2.62 (s, 3H). Mol. Wt: 600.86-boronic acid and 682.00-boronate ester; MS (ES+): m/z: 601.15 [MH⁺] (boronic acid), 683.15 [MH⁺] (boronate ester), HPLC purity: 21 and 71.96%(mixture of boronic acid & ester) at Max plot.

Following the general procedure for synthesis of (4-(2-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-2-oxoethyl)phenyl)boronic acid, the compounds below were synthesized.

### Synthesis of (4-((E)-3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-3-oxoprop-1-en-1-yl)phenyl)boronic acid (BRD-E-29)

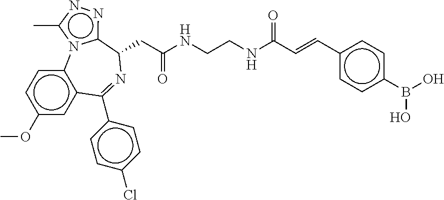

A solution of 3-(4-boronophenyl)acrylic acid (21.7 mg, 0.11 mmol) in DCM (10 mL) was charged with EDCI (32 mg, 0.17 mmol), DMAP (16 mg, 0.13 mmol), and HOBt (23 mg, 0.17 mmol) and stirred at rt for 10 min. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and purified using the same conditions as above general procedure resulting in 21 mg, 30% yield of the title compound as a white solid. ¹H NMR (400 MHz, Methanol-d₄) δ 7.81-7.60 (m, 3H), 7.58-7.44 (m, 5H), 7.42-7.34 (m, 3H), 6.92-6.86 (m, 1H), 6.62 (d, J=16.1 Hz, 1H), 4.67 (dt, J=7.9, 4.0 Hz, 1H), 3.80 (s, 3H), 3.53-3.27 (m, 6H), 2.69 (s, 3H). Mol. Wt: 612.87, MS (ES+): m/z: 635.12 [M+Na], HPLC purity:—97.72% (Max plot).

### Synthesis of (3-((E)-3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-3-oxoprop-1-en-1-yl)phenyl)boronic acid (BRD-E-30)

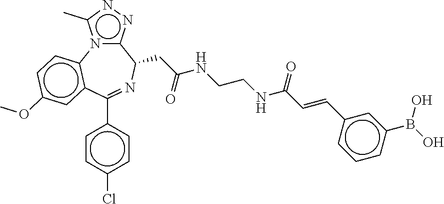

A solution of 3-(3-boronophenyl)acrylic acid (21.7 mg, 0.11 mmol) in DCM (10 mL) was charged with EDCI (32 mg, 0.17 mmol), DMAP (16 mg, 0.13 mmol), and HOBt (23 mg, 0.17 mmol) and stirred at rt for 10 min. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and purified using the same conditions as above general procedure resulting in 15 mg, 21.5% yield of the title compound as a white solid. ¹H NMR (400 MHz, DMSO-d₆) δ 8.34 (d, J=6.7 Hz, 2H), 8.22 (s, 1H), 8.15 (t, J=5.5 Hz, 2H), 7.99 (s, 2H), 7.80 (q, J=9.1, 5.9 Hz, 2H), 7.68-7.33 (m, 3H), 6.87 (d, J=3.0 Hz, 1H), 6.65 (d, J=15.9 Hz, 1H), 4.55-4.47 (m, 1H), 3.78 (s, 3H), 3.33-3.13 (m, 6H), 2.57 (s, 3H). Mol. Wt: 612.87, MS (ES+): m/z: 613.35 [MH⁺], HPLC purity:—81% (Max plot).

### Synthesis of (3′-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)-[1,1′-biphenyl]-3-yl)boronic acid (BRD-E-31)

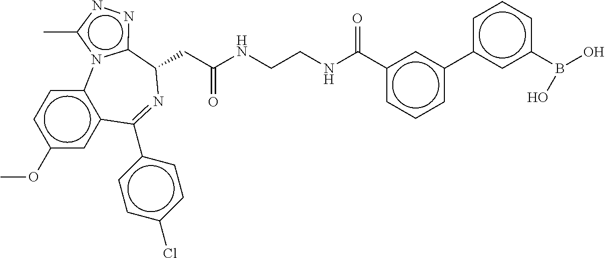

A solution of 3′-(4,4,5,5-tetramethyl-1,3,2-dioxaborolan-2-yl)-[1,1′-biphenyl]-3-carboxylic acid (36.9 mg, 0.11 mmol) in DCM (10 mL) was charged with EDCI (32 mg, 0.17 mmol), DMAP (16 mg, 0.13 mmol), and HOBt (23 mg, 0.17 mmol) and stirred at rt for 10 min. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and purified using the same conditions as above general procedure resulting in 17 mg, 20% yield of the title compound as a white solid as mixture of boronic acid and ester. ¹H NMR (400 MHz, Methanol-d₄) δ 8.02 (s, 1H), 7.93 (s, 1H), 7.85-7.65 (m, 7H), 7.61-7.35 (m, 3H), 7.30 (t, J=7.0 Hz, 3H), 7.18 (dd, J=8.7, 3.0 Hz, 1H), 6.54 (d, J=3.1 Hz, 1H), 4.80-4.78 (m, 1H), 3.76-3.58 (m, 2H), 3.53-3.41 (m, 4H), 3.29 (s, 3H), 2.64 (s, 3H), 1.32 (s, 2H). Mol. Wt: 662.93, boronic acid and 745.07, boronate ester, MS (ES+): m/z: 663.30 [MH⁺] (boronic acid), 745.20 [MH⁺] (boronate ester), HPLC purity: 32.6 & 64.9% (mixture of boronic acid & ester) at Max plot.

### Synthesis of (3-(2-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-2-oxoethyl)phenyl)boronic acid (BRD-E-38)

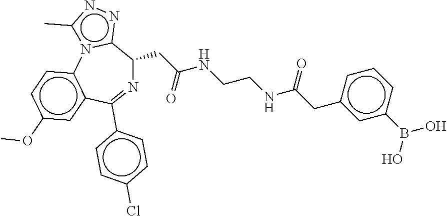

A solution of 2-(3-(4,4,5,5-tetramethyl-1,3,2-dioxaborolan-2-yl)phenyl)acetic acid (29.8 mg, 0.11 mmol) in DCM (10 mL) was charged with EDCI (32 mg, 0.17 mmol), DMAP (16 mg, 0.13 mmol), and HOBt (23 mg, 0.17 mmol) and stirred at rt for 10 min. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and was purified using the same conditions as above general procedure resulting in 12 mg, 15.5% yield of the title compound as a white solid as mixture of boronic acid and ester. ¹H NMR (400 MHz, DMSO-d₆) δ 7.68-7.54 (m, 9H), 7.37 (s, 2H), 2.81 (s, 3H), 4.57-4.37 (m, 1H), 3.40-3.07 (m, 4H), 2.51 (s, 3H), 2.43-2.41 (m, 1H), 2.29-2.21 (m, 3H). Mol. Wt: 600.86; boronic acid & 683.00; boronate ester; MS (ES+): m/z: 623.20 [M+Na], (boronic acid), 683.00 [MH⁺] (boronate ester), HPLC purity: 60.8 and 35.1% (mixture of boronic acid & ester) at Max plot.

### Synthesis of 2-((4S)-6-(4-chlorophenyl)-8-methoxy-1-methyl-4H-benzo[f][1,2,4]triazolo[4,3-a][1,4]diazepin-4-yl)-N-(2-(2-(3,4-dihydroxyphenyl)acetamido)ethyl)acetamide (BRD-N-30)

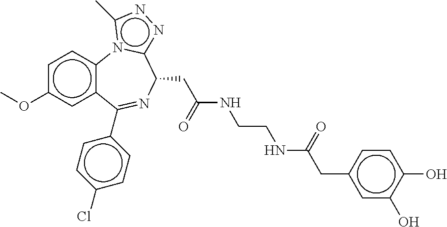

A solution of 2-(3,4-dihydroxyphenyl)acetic acid (22.9 mg, 0.13 mmol) in DCM (10 mL) was charged with HATU (77.9 mg, 0.20 mmol) and DIPEA (17.5 mg, 0.20 mmol) and stirred at rt for 10 minutes. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and stirred at room temperature overnight. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 10 mg, 12.5% yield of the title compound as a white solid. ¹H NMR (400 MHz, DMSO-d₆) δ 8.26 (s, 1H), 7.91 (s, 1H), 7.79 (d, J=8.9 Hz, 1H), 7.50 (q, J=8.2 Hz, 1H), 7.39 (d, J=9.0 Hz, 1H), 6.88 (s, 1H), 6.68-6.58 (m, 5H), 6.47 (d, J=8.1 Hz, 1H), 4.50 (t, J=8.1 Hz, 1H), 3.79 (s, 3H), 3.40-3.30 (m, 6H), 3.18 (q, J=16.4, 13.9 Hz, 2H), 2.56 (s, 3H). Mol. Wt: 589.04; MS (ES+): m/z: 589.25 [MH⁺], HPLC purity:—98.67% (Max plot).

Synthesis of (S,E)-N-(2-(2-(6-(4-chlorophenyl)-8-methoxy-1-methyl-4H-benzo[f][1,2,4]triazolo[4,3-a][1,4]diazepin-4-yl)acetamido)ethyl)-3-(3,4-dihydroxyphenyl)acrylamide (BRD-N-32):

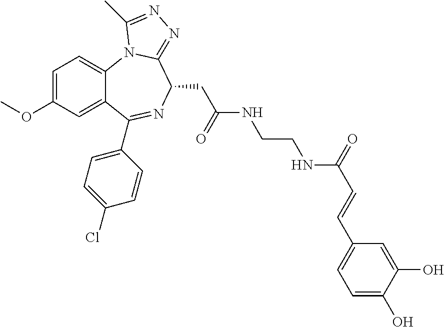 [SMILES: COc1ccc2c(c1)C(c1ccc(Cl)cc1)=N[C@@H](CC(=O)NCCNC(=O)/C=C/c1ccc(O)c(O)c1)c1nnc(C)n1-2]

A solution of (S)—N-(2-aminoethyl)-2-(6-(4-chlorophenyl)-8-methoxy-1-methyl-4H-benzo[f][1,2,4]triazolo[4,3-a][1,4]diazepin-4-yl)acetamide (100 mg, 0.227 mmol.) and 3,4-dihydroxy cinnamic acid (74 mg, 0.271 mmol.) in DMF (3 ml) was charged with EDCI (52 mg, 0.273 mmol), HOBt (32 mg, 0.273 mmol) and triethyl amine (34 mg, 0.340 mmol) and stirred at room temperature overnight. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 20 mg, 14.7% yield of the title compound as white Solid. ¹H NMR (400 MHz, DMSO-d₆) δ 8.33 (t, J=5.0 Hz, 1H), 8.01 (t, J=5.4 Hz, 1H), 7.81 (dd, J=15.1, 8.6 Hz, 1H), 7.56-7.43 (m, 2H), 7.38 (dd, J=8.9, 2.9 Hz, 1H), 7.25 (d, J=15.7 Hz, 1H), 6.95 (d, J=2.1 Hz, 1H), 6.89-6.80 (m, 2H), 6.74 (d, J=8.2 Hz, 1H), 6.34 (d, J=15.7 Hz, 1H), 4.49 (dd, J=8.3, 5.7 Hz, 1H), 3.78 (s, 3H), 3.32-3.12 (m, 4H), 2.54 (s, 3H). Mol Wt:—600.19, MS (ES+): m/z: 623.0 [M+Na], HPLC purity: 94.80% (Max plot).

### Synthesis of (S)—N-(2-(2-(6-(4-chlorophenyl)-8-methoxy-1-methyl-4H-benzo[f][1,2,4]triazolo[4,3-a][1,4]diazepin-4-yl)acetamido)ethyl)-3-(3,4-dihydroxyphenyl)propanamide (BRD-N-33)

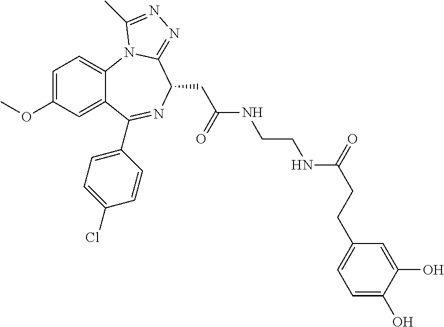 [SMILES: COc1ccc2c(c1)C(c1ccc(Cl)cc1)=N[C@@H](CC(=O)NCCNC(=O)CCc1ccc(O)c(O)c1)c1nnc(C)n1-2]

A solution of (S)—N-(2-aminoethyl)-2-(6-(4-chlorophenyl)-8-methoxy-1-methyl-4H-benzo[f][1,2,4]triazolo[4,3-a][1,4]diazepin-4-yl)acetamide (100 mg, 0.227 mmol.) and 3-(3,4-dihydroxyphenyl)propanoic acid (49.8 mg, 0.277 mmol) in DMF (3 ml) was charged with EDCI (52 mg, 0.273 mmol), HOBt (37 mg, 0.273 mmol.), and triethylamine (34 mg, 0.340 mmol) and stirred at room temperature overnight. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 27 mg, 20% t of the title compound as white Solid. ¹H NMR (400 MHz, DMSO-d6) δ 7.85-7.64 (m, 4H), 7.56-7.44 (m, 3H), 6.64-6.52 (m, 3H), 4.13 (dd, J=5.7, 3.3 Hz, 1H), 3.78 (s, 3H), 3.18-3.09 (m, 8H), 2.59 (q, J=14.4, 11.1 Hz, 2H), 2.25 (s, 3H). Mol Wt: 602.80, MS (ES+): m/z: 602.85 [MH⁺], HPLC purity: 99.41% (Max plot).

### ((S)—N-(2-aminoethyl)-2-(6-(4-chlorophenyl)-8-methoxy-1-methyl-4H-benzo[f][1,2,4]triazolo[4,3-a][1,4]diazepin-4-yl)acetamide

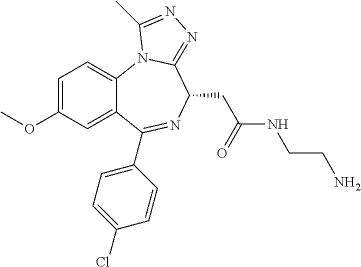 [SMILES: COc1ccc2c(c1)C(c1ccc(Cl)cc1)=N[C@@H](CC(=O)NCCN)c1nnc(C)n1-2]

A solution of (S)-tert-butyl(2-(2-(6-(4-chlorophenyl)-8-methoxy-1-methyl-4H-benzo[f][1,2,4]triazolo[4,3-a][1,4]diazepin-4-yl)acetamido)ethyl)carbamate (2.8 g, 0.52 mmol) in DCM (50 mL) was charged with TFA (5 mL) and stirred at rt for 12 h. The reaction mixture was evaporated under reduced pressure to obtain a residue which was redissolved in DCM (10 mL) and charged with powdered KOH to adjust solution to pH˜8-9 and filtered through a pad of celite and the filtrate was concentrated in vacuo resulting in a crude product which was purified by column chromatography on silica gel (100-200 mesh), eluting with 4% methanol in chloroform to afford 1.7 g, 74.59% yield of title compound as a yellow solid. Mol Wt: 438.09, MS (ES+): m/z: 439.15 [MH⁺].

### (S)-tert-butyl(2-(2-(6-(4-chlorophenyl)-8-methoxy-1-methyl-4H-benzo[f][1,2,4]triazolo[4,3-a][1,4]diazepin-4-yl)acetamido)ethyl)carbamate

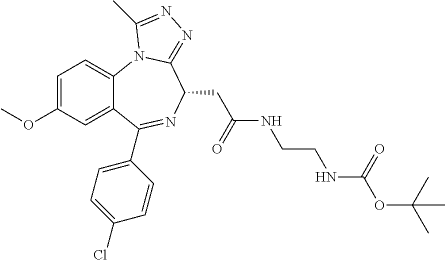 [SMILES: COc1ccc2c(c1)C(c1ccc(Cl)cc1)=N[C@@H](CC(=O)NCCNC(=O)OC(C)(C)C)c1nnc(C)n1-2]

A suspension of (S)-2-(6-(4-chlorophenyl)-8-methoxy-1-methyl-4H-benzo[f][1,2,4]triazolo[4,3-a][1,4]diazepin-4-yl)acetic acid (2.5 g, 6.3 mmol) in DCM (6 mL) was charged with EDCI (1.79 g, 9.4 mmol), 4-DMAP (1.1 g, 9.4 mmol), and HOBt (1.2 g, 9.4 mmol) and stirred at rt for 10 minutes. This solution was charged with tert-butyl(2-aminoethyl) carbamate (1.5 g, 9.4 mmol) and stirred at room temperature overnight. The reaction mixture was partitioned between DCM and H₂O and separated and the aqueous layer was re-extracted with DCM (3×10 mL) and the combined organic fractions were dried over anhydrous Na₂SO₄, filtered and concentrated in vacuo resulting in a crude product which was purified by column chromatography on silica gel (100-200 mesh), eluting with 1% methanol in chloroform to afford 3.30 g, 85.5% yield of the title compound as an off white solid. Mol Wt: 539.03, MS (ES+): m/z: 539.15 [MH⁺].

### Example 356

Monomers were synthesized according to the procedures described below.

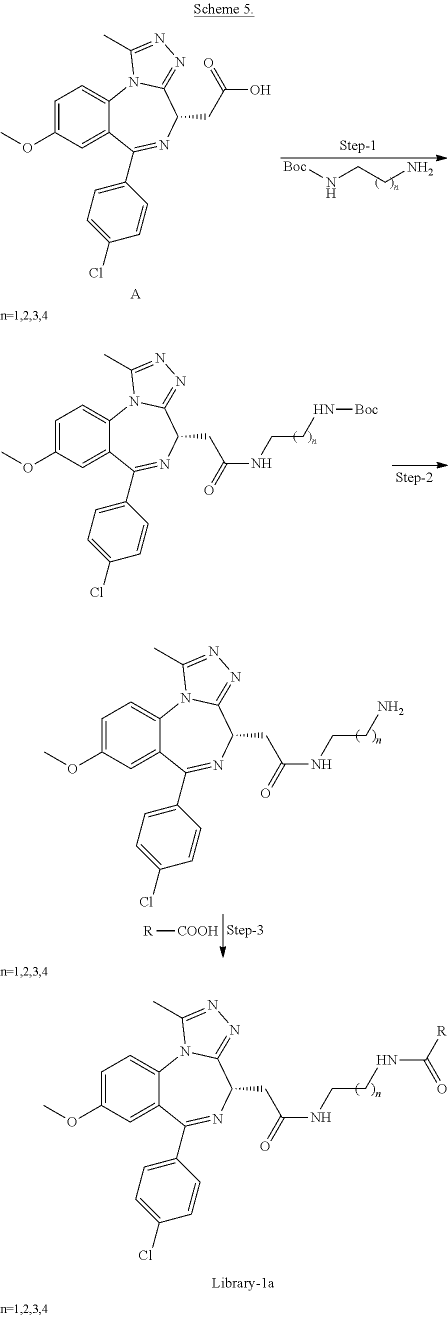

### Synthesis of (4-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)boronic acid (BRD-E-01)

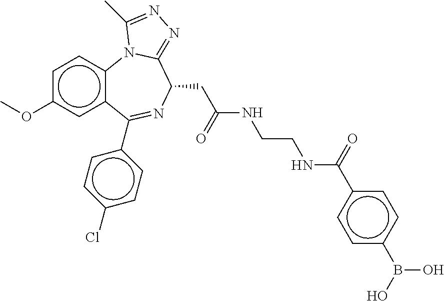

A solution of 4-boronobenzoic acid (9 mg, 0.05 mmol) in DCM (10 mL) was charged with EDCI (13 mg, 0.06 mmol), DMAP (3.3 mg, 0.02 mmol), and stirred at rt for 10 minutes. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (20 mg, 0.12 mmol) and stirred at room temperature overnight. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford in 10 mg, 38% yield of the title compound as a white solid. ¹H NMR (400 MHz, CD₃CN) δ 7.85-7.67 (m, 4H), 7.60 (d, J=8.9 Hz, 1H), 7.50 (d, J=7.9 Hz, 2H), 7.38-7.30 (m, 3H), 6.82 (d, J=2.8 Hz, 1H), 4.62 (t, J=8.0 Hz, 1H), 3.80 (s, 3H), 3.52-3.40 (m, 4H), 3.33-3.23 (m, 2H), 3.03 (s, 3H). Mol. Wt: 586.83; MS (ES+): m/z 587.30 0 [MH⁺], HPLC purity: 91.5% at 254 nm.

Following the general procedure for synthesis of (4-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)boronic acid, the compounds below were synthesized.

### Synthesis of (3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)boronic acid (BRD-E-02)

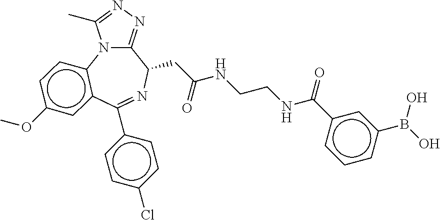

A solution of 3-boronobenzoic acid (9 mg, 0.05 mmol) in DCM (10 mL) were charged with EDCI (13 mg, 0.06 mmol), DMAP (3.3 mg, 0.02 mmol), and stirred at rt for 10 minutes and charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (20 mg, 0.12 mmol) and purified according to the same conditions to purify (4-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 6 mg, 23% yield of the title compound as a white solid. ¹H NMR (400 MHz, Methanol-d₄) δ 8.06 (s, 1H), 7.91-7.67 (m, 3H), 7.62-7.50 (m, 1H), 7.52-7.30 (m, 6H), 6.78 (d, J=2.8 Hz, 1H), 4.68 (t, J=9.0, Hz, 1H), 3.82 (s, 3H), 3.63-3.36 (m, 6H), 2.67 (s, 3H). Mol. Wt: 586.30; MS (ES+): m/z 587.30 [MH⁺], HPLC purity: 91.27% (220 nm).

### Synthesis of (4-((3-(2-((4S)-6-(4-chlorophenyl)-8-methoxy-1-methyl-4H-benzo[f][1,2,4]triazolo[4,3-a][1,4]diazepin-4-yl)acetamido)propyl)carbamoyl)phenyl)-boronic acid (BRD-E-03)

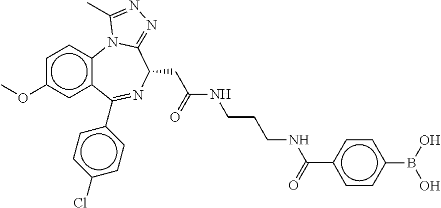

A solution of 4-boronobenzoic acid (50 mg, 0.11 mmol) in DCM (10 mL) were charged with EDCI (32 mg, 0.16 mmol), DMAP (7 mg, 0.05 mmol), and stirred at rt for 10 minutes and charged with N-(3-aminopropyl)-2-((4S)-6-(4-chlorophenyl)-8-methoxy-1-methyl-4H-benzo[f][1,2,4]triazolo[4,3-a][1,4]diazepin-4-yl)acetamide (21.9 mg, 0.13 mmol) and purified according to the same conditions used to purify (4-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 13 mg, 19.6% yield of the title compound as off white solid. ¹H NMR (400 MHz, DMSO-d₆) δ 8.43 (s, 1H), 8.26 (s, 1H), 7.88-7.72 (m, 4H), 7.55-7.33 (m, 6H), 6.87 (d, J=2.7 Hz, 1H), 4.49 (dd, J=8.7, 4.9 Hz, 1H), 3.78 (s, 3H), 3.35-3.10 (m, 4H), 2.67 (s, 3H), 1.74-1.69 (m, 2H), 1.53-1.45 (m, 2H). Mol. Wt: 600.86; MS (ES+): m/z 601.40 [MH⁺], HPLC purity: 98.26% (Max plot).

### Synthesis of (3-((3-(2-((4S)-6-(4-chlorophenyl)-8-methoxy-1-methyl-4H-benzo[f][1,2,4]triazolo[4,3-a][1,4]diazepin-4-yl)acetamido)propyl)carbamoyl)phenyl)-boronic acid (BRD-E-04)

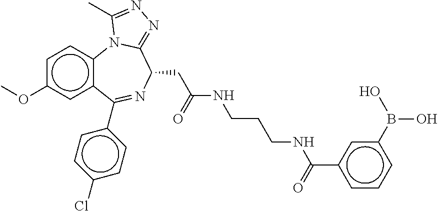

A solution of 3-boronobenzoic acid (50 mg, 0.11 mmol) in DCM (10 mL) was charged with EDCI (32 mg, 0.16 mmol) and DMAP (7 mg, 0.05 mmol) and stirred at rt for 10 minutes then charged with N-(3-aminopropyl)-2-((4S)-6-(4-chlorophenyl)-8-methoxy-1-methyl-4H-benzo[f][1,2,4]triazolo[4,3-a][1,4]diazepin-4-yl)acetamide (21.9 mg, 0.13 mmol) and purified according to the same conditions used to purify (4-((2-((2(4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 10 mg, 15.1% yield of the title compound as off white solid. ¹H NMR (400 MHz, CD₃CN) δ 8.20 (s, 1H), 7.92-7.78 (m, 2H), 7.62 (d, J=9.0 Hz, 1H), 7.58-7.51 (m, 2H), 7.50-7.29 (m, 4H), 7.15 (t, J=6.1 Hz, 1H), 6.94 (d, J=2.9 Hz, 1H), 4.65 (t, J=7.1 Hz, 1H), 3.79 (s, 3H), 3.48-3.22 (m, 4H), 2.68 (s, 3H), 1.70-1.65 (m, 2H), 1.38-1.14 (m, 2H). Mol. Wt: 600.86; MS (ES+): m/z 601.30 [MH⁺], HPLC purity: 96.42% (Max plot).

### Synthesis of (4-((4-(2-((4S)-6-(4-chlorophenyl)-8-methoxy-1-methyl-4H-benzo[f][1,2,4]triazolo[4,3-a][1,4]diazepin-4-yl)acetamido)butyl)carbamoyl)phenyl)boronic acid (BRD-E-05)

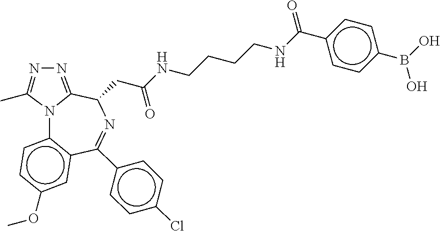

A solution of 4-boronobenzoic acid (50 mg, 0.10 mmol) in DCM (10 mL) was charged with EDCI (31 mg, 0.16 mmol) and DMAP (6.5 mg, 0.05 mmol) and stirred at rt for 10 minutes then charged with (S)—N-(4-aminobutyl)-2-(6-(4-chlorophenyl)-8-methoxy-1-methyl-4H-benzo[f][1,2,4]triazolo[4,3-a][1,4]diazepin-4-yl)acetamide (21 mg, 0.12 mmol) and purified according to the same conditions used to purify (4-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 16 mg, 24.6% yield of the title compound as off white solid. ¹H NMR (400 MHz, DMSO-d₆) δ 8.49 (s, 1H), 8.19 (s, 1H), 7.88-7.74 (m, 5H), 7.54-7.33 (m, 5H), 6.86 (s, 1H), 4.48 (dd, J=8.2, 5.1 Hz, 1H), 3.78 (s, 3H), 3.28-3.03 (m, 5H), 2.60 (s, 3H), 1.67-1.54 (m, 4H), 1.24-1.120 (m, 1H). Mol. Wt: 614.89; MS (ES+): m/z 615.50 [MH⁺], HPLC purity:—97.38% (254 nm).

### Synthesis of (3-((4-(2-((4S)-6-(4-chlorophenyl)-8-methoxy-1-methyl-4H-benzo[f][1,2,4]triazolo[4,3-a][1,4]diazepin-4-yl)acetamido)butyl)carbamoyl)phenyl)boronic acid (BRD-E-06)

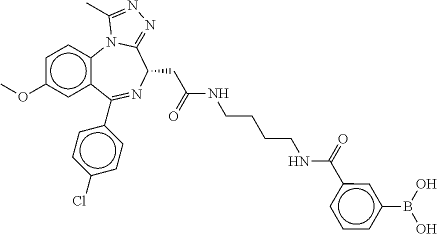

A solution of 3-boronobenzoic acid (50 mg, 0.10 mmol) in DCM (10 mL) was charged with EDCI (31 mg, 0.16 mmol) and DMAP (6.5 mg, 0.05 mmol) and stirred at rt for 10 minutes then charged with (S)—N-(4-aminobutyl)-2-(6-(4-chlorophenyl)-8-methoxy-1-methyl-4H-benzo[f][1,2,4]triazolo[4,3-a][1,4]diazepin-4-yl)acetamide (21 mg, 0.12 mmol). and purified according to the same conditions used to purify (4-((2-((2(4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 10 mg, 15.3% yield of the title compound as off white solid. ¹H NMR (400 MHz, CD₃CN) δ 8.22 (s, 1H), 7.84 (dd, J=8.9, 2.9 Hz, 2H), 7.54-7.33 (m, 5H), 6.90-6.85 (m, 3H), 4.56 (t, J=9.0, Hz, 1H), 3.79 (s, 3H), 3.40-3.22 (m, 5H), 2.56 (s, 3H), 1.70-1.56 (m, 4H), 1.26-1.20 (m, 1H). Mol. Wt: 614.89; MS (ES+): m/z 615.35 [MH⁺], HPLC purity: 89.07% (220 nm).

### Synthesis of (4-((5-(2-((4S)-6-(4-chlorophenyl)-8-methoxy-1-methyl-4H-benzo[f][1,2,4]triazolo[4,3-a][1,4]diazepin-4-yl)acetamido)pentyl)carbamoyl)phenyl)-boronic acid (BRD-E-07)

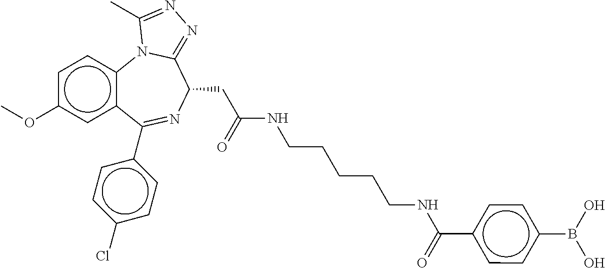

A solution of 4-boronobenzoic acid (50 mg, 0.10 mmol) in DCM (10 mL) was charged with EDCI (30 mg, 0.15 mmol) and DMAP (6.2 mg, 0.05 mmol) stirred at rt for 10 minutes then charged with N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (21 mg, 0.12 mmol) and purified according to the same conditions used to purify (4-((2-((2-(4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 16 mg, 24.6% yield of the title compound as off white solid. ¹H NMR (400 MHz, DMSO-d₆) δ 8.43 (s, H), 8.19 (s, 1H), 7.84-7.75 (m, 5H), 7.53-7.48 (m, 4H), 7.37 (dd, J=9.2, 3.3 Hz, 1H), 6.87 (d, J=2.6 Hz, 1H), 4.48 (dd, J=8.5, 5.3 Hz, 1H), 3.79 (s, 3H), 3.25-3.05 (m, 5H), 2.57 (s, 3H), 1.52-1.35 (m, 5H), 1.26-1.21 (m, 2H). Mol. Wt: 628.91; MS (ES+): m/z 629.70 [MH⁺], HPLC purity: 95.06% (254 nm).

### Synthesis of (3-((5-(2-((4S)-6-(4-chlorophenyl)-8-methoxy-1-methyl-4H-benzo[f][1,2,4]triazolo[4,3-a][1,4]diazepin-4-yl)acetamido)pentyl)carbamoyl)phenyl)-boronic acid (BRD-E-08)

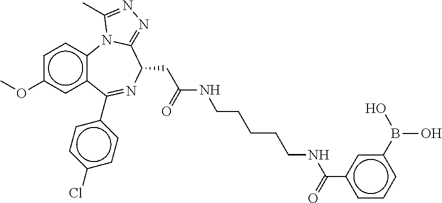

A solution of 3-boronobenzoic acid (50 mg, 0.10 mmol) in DCM (10 mL) were charged with EDCI (30 mg, 0.15 mmol) and DMAP (6.2 mg, 0.05 mmol) and stirred at rt for 10 minutes then charged with N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (21 mg, 0.12 mmol) and purified according to the same conditions used to purify (4-((2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamoyl)phenyl)-boronic acid resulting in 20 mg, 30.7% yield of the title compound as off white solid. ¹H NMR (400 MHz, DMSO-d₆) δ 8.38 (s, 1H), 8.22 (s, 1H), 7.92-7.76 (m, 7H), 7.50-7.40 (m, H), 7.38 (d, J=8.5 Hz, 1H), 6.87 (d, J=2.6 Hz, 1H), 4.49 (d, J=8.9 Hz, 1H), 3.79 (s, 3H), 3.31-3.22 (m, 5H), 3.18-3.09 (m, 1H), 2.63 (s, 3H), 1.55-1.48 (m, 4H), 1.39-1.32 (m, 2H). Mol. Wt: 628.91; MS (ES+): m/z 629.25 [MH⁺], HPLC purity:—94.82% (220 nm).

### Synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-3,4-dihydroxybenzamide (BRD-N-01)

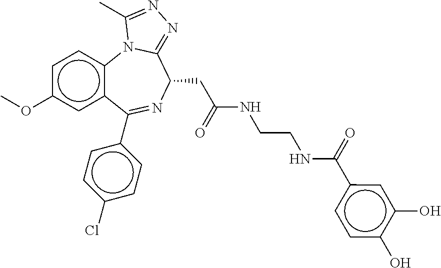

A solution of 3,4-dihydroxybenzoic acid (43 mg, 0.28 mmol) in DMF (5 mL) was charged with EDCI (32 mg, 0.17 mmol), DIPEA (29 mg, 0.22 mmol), and HOBt (23 mg, 0.17 mmol) and stirred at rt for 10 minutes then charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and stirred overnight at room temperature. The reaction mixture was partitioned between DCM and H₂O and separated and the aqueous layer was re-extracted with DCM (3×10 mL) and the combined organic fractions were dried over anhydrous Na₂SO₄, filtered and concentrated in vacuo resulting in a crude product which was purified by preparative HPLC to afford 10 mg, 15.3% yield of the corresponding title compound as an off white solid. ¹H NMR (400 MHz, Methanol-d4) δ 7.68 (d, J=9.1 Hz, 1H), 7.47 (d, J=8.3 Hz, 2H), 7.42-7.30 (m, 3H), 7.25-7.12 (m, 2H), 6.84-6.65 (m, 2H), 4.68-4.58 (m, 1H), 3.81 (s, 3H), 3.58-3.32 (m, 6H), 2.61 (s, 3H). Mol. Wt: 575.01; MS (ES+): m/z 575.45 [MH⁺], HPLC purity: 96.57% (254 nm).

The following compounds have been synthesized and purified using the general procedure described above for the synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-3,4-dihydroxybenzamide.

### Synthesis of N-(3-(2-((4S)-6-(4-chlorophenyl)-8-methoxy-1-methyl-4H-benzo[f][1,2,4]triazolo[4,3-a][1,4]diazepin-4-yl)acetamido)propyl)-3,4-dihydroxybenzamide (BRD-N-03)

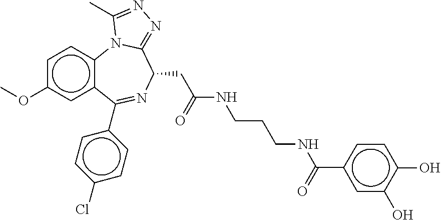

A solution of 3,4-dihydroxybenzoic acid (42 mg, 0.27 mmol) in DMF (5 mL) were charged with EDCI (31 mg, 0.16 mmol), DIPEA (28 mg, 0.21 mmol), and HOBt (22 mg, 0.16 mmol) and stirred at rt for 10 minutes then charged with N-(3-aminopropyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) resulting in 9 mg, 13.8% yield of the title compound as off white solid. ¹H NMR (400 MHz, Methanol-d₄) δ 7.74 (d, J=9.1 Hz, 1H), 7.59-7.46 (m, 2H), 7.43-7.32 (m, 3H), 7.29-7.21 (m, 1H), 7.23-7.16 (m, 1H), 6.93 (d, J=3.0 Hz, 1H), 6.78 (dd, J=7.8, 4.4 Hz, 1H), 4.68 (dd, J=8.7, 5.6 Hz, 1H), 3.82 (s, 3H), 3.49-3.22 (m, 6H), 2.69 (s, 3H), 1.83-1.78 (m, 2H). Mol. Wt: 589.04; MS (ES+): m/z 589.55 [MH⁺], HPLC purity: 94.63% (Max plot).

### Synthesis of N-(4-(2-((4S)-6-(4-chlorophenyl)-8-methoxy-1-methyl-4H-benzo[f][1,2,4]triazolo[4,3-a][1,4]diazepin-4-yl)acetamido)butyl)-3,4-dihydroxybenzamide (BRD-N-05)

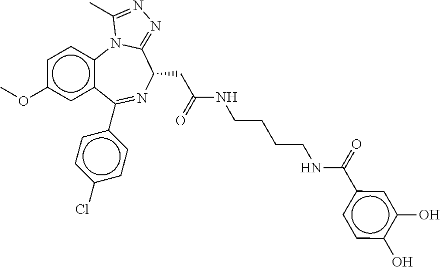

A solution of 3,4-dihydroxybenzoic acid (41 mg, 0.26 mmol) in DMF (5 mL) was charged with EDCI (30 mg, 0.15 mmol), DIPEA (27 mg, 0.20 mmol), and HOBt (21 mg, 0.15 mmol) and stirred at rt for 10 minutes then charged with N-(4-aminobutyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) resulting in 20 mg, 31% yield of the title compound as off white solid. ¹H NMR (400 MHz, Methanol-d₄) δ 8.16 (s, 1H), 7.71 (d, J=8.9 Hz, 1H), 7.53 (d, J=8.2 Hz, 2H), 7.42-7.33 (m, 3H), 7.28 (d, J=2.3 Hz, 1H), 7.23-7.16 (m, 1H), 6.91 (d, J=2.9 Hz, 1H), 6.78 (d, J=8.3 Hz, 1H), 4.63 (dd, J=8.9, 5.3 Hz, 1H), 3.82 (s, 3H), 3.41 (dd, J=14.6, 8.3 Hz, 3H), 3.27 (dd, J=15.0, 5.4 Hz, 1H), 2.63 (s, 3H), 1.72-1.60 (m, 4H), 1.31-1.25 (m, 2H). Mol. Wt: 603.07; MS (ES+): m/z 603.50 [MH⁺], HPLC purity: 95.78% (254 nm).

### N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)-3,4-dihydroxybenzamide (BRD-N-07)

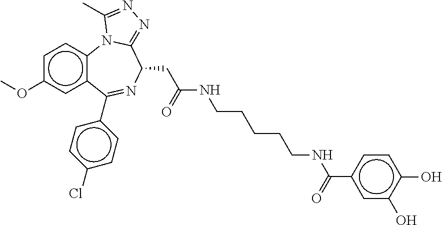

A solution of 3,4-dihydroxybenzoic acid (40 mg, 0.25 mmol) in DMF (5 mL) was charged with EDCI (29 mg, 0.14 mmol), DIPEA (26 mg, 0.19 mmol), and HOBt (20 mg, 0.14 mmol) and stirred at rt for 10 minutes then charged with N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.10 mmol) resulting in 10 mg, 15.5% yield of the title compound as off white solid. ¹H NMR (400 MHz, Methanol-d₄) δ 7.70 (d, J=8.8 Hz, 1H), 7.57-7.43 (m, 2H), 7.44-7.32 (m, 3H), 7.30-7.15 (m, 2H), 6.90 (s, 1H), 6.75 (d, J=8.0 Hz, 1H), 4.61 (dd, J=8.6, 5.1 Hz, 1H), 4.00-3.91 (m, 1H), 3.84-3.79 (m, 3H), 3.49-3.32 (m, 2H), 2.60 (s, 3H), 1.63-1.43 (m, 3H), 1.29-1.22 (m, 2H). Mol. Wt: 617.09; MS (ES+): m/z 618.65 [MH⁺], HPLC purity: 93.54% (254 nm).

### Synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-2,3-dihydroxybenzamide (BRD-N-02)

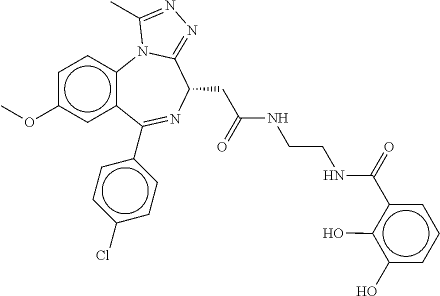

A solution of 2,3-dihydroxybenzoic acid (52 mg, 0.34 mmol) in DCM (10 mL) was charged with TEA (138 mg, 1.3 mmol) and stirred at 0° C. for 5 minutes. This solution was dropwise charged with TMS-Cl (110 mg, 1.0 mmol) at 0° C. then allowed to reach room temperature and stirred for 6 h. The reaction mixture was then charged with EDCI (32 mg, 0.17 mmol) and DMAP (20 mg, 0.17 mmol) and stirred at rt for 10 minutes then charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and stirred overnight at room temperature. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 23 mg, 35.3% yield of the corresponding title compound as an off white solid. ¹H NMR (400 MHz, Methanol-d₄) δ 7.73-7.66 (m, 1H), 7.52-7.45 (m, 2H), 7.43-7.28 (m, 3H), 7.11 (dd, J=7.8, 1.9 Hz, 1H), 6.93-6.82 (m, 2H), 6.63-655 (m, 1H), 4.67 (dd, J=8.4, 5.6 Hz, 1H), 3.83 (s, 3H), 3.57-3.26 (m, 6H), 2.69 (s, 3H). Mol. Wt: 575.01; MS (ES+): m/z 575.35 [MH⁺], HPLC purity: 95.43% (254 nm).

The following compounds have been synthesized and purified using the general procedure described above for the synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-2,3-dihydroxybenzamide.

### Synthesis of N-(3-(2-((4S)-6-(4-chlorophenyl)-8-methoxy-1-methyl-4H-benzo[f][1,2,4]triazolo[4,3-a][1,4]diazepin-4-yl)acetamido)propyl)-2,3-dihydroxybenzamide (BRD-N-04)

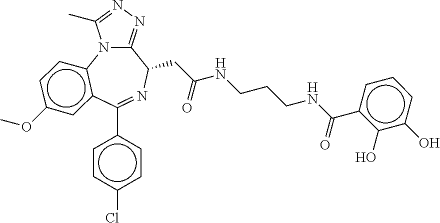

A solution of 2,3-dihydroxybenzoic acid (49 mg, 0.32 mmol) in DCM (10 mL) was charged with TEA (136 mg, 1.28 mmol) and stirred at 0° C. for 5 minutes then dropwise charged with TMS-Cl (104 mg, 0.96 mmol) and stirred at room temperature for 6 h. The reaction mixture was then charged with EDCI (30 mg, 0.17 mmol) and DMAP (19 mg, 0.16 mmol) and stirred at rt for 10 minutes then charged with N-(3-aminopropyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) resulting in 25 mg, 38.4% yield of the title compound as off white solid. ¹H NMR (400 MHz, Methanol-d₄) δ 7.70 (dd, J=9.1, 1.8 Hz, 1H), 7.57-7.50 (m, 2H), 7.40-7.32 (m, 3H), 7.19 (dd, J=8.2, 1.4 Hz, 1H), 6.91 (dt, J=5.1, 2.1 Hz, 2H), 6.69 (t, J=7.9 Hz, 1H), 4.64 (dd, J=8.8, 5.5 Hz, 1H), 3.81 (s, 3H), 3.49-3.31 (m, 6H), 2.63 (s, 3H), 1.88-1.70 (m, 2H). Mol. Wt: 589.04; MS (ES+): m/z 589.45 HPLC purity: 97.37% (Max plot).

### Synthesis of N-(4-(2-((4S)-6-(4-chlorophenyl)-8-methoxy-1-methyl-4H-benzo[f][1,2,4]triazolo[4,3-a][1,4]diazepin-4-yl)acetamido)butyl)-2,3-dihydroxybenzamide (BRD-N-06)

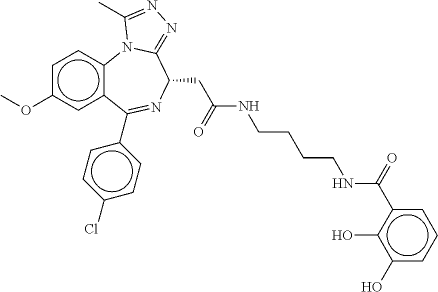

A solution of 2,3-dihydroxybenzoic acid (49 mg, 0.39 mmol) in DCM (10 mL) was charged with TEA (129 mg, 1.2 mmol) and stirred at 0° C. for 5 minutes then dropwise charged with TMS-Cl (104 mg, 0.96 mmol) and stirred at room temperature for 6 h. The reaction was then charged with EDCI (30 mg, 0.16 mmol) and DMAP (19 mg, 0.16 mmol) and stirred at room temperature for 10 minutes and then charged with N-(4-aminobutyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) resulting in 25 mg, 38.7% yield of the title compound as off white solid. ¹H NMR (400 MHz, Methanol-d₄) δ 7.67 (d, J=9.0 Hz, 1H), 7.51 (d, J=8.1 Hz, 2H), 7.34 (dd, J=9.8, 5.2 Hz, 3H), 7.19 (d, J=8.1 Hz, 1H), 6.94-6.86 (m, 2H), 6.67 (t, J=7.9 Hz, 1H), 4.62 (dd, J=8.9, 5.3 Hz, 1H), 3.79 (s, 3H), 3.45-3.20 (m, 6H), 2.61 (s, 3H), 1.65-1.57 (m, 2H), 1.28-1.20 (m, 2H). Mol. Wt: 603.04; MS (ES+): m/z MS (ES+): m/z 603.40 [MH⁺], HPLC purity: 98.04% (Max plot).

### Synthesis of N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)-2,3-dihydroxybenzamide (BRD-N-08)

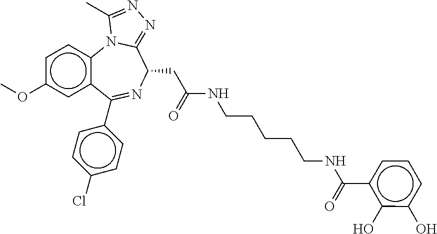

A solution of 2,3-dihydroxybenzoic acid (57 mg, 0.37 mmol) in DCM (10 mL) was charged with TEA (151 mg, 1.4 mmol) and stirred at 0° C. for 5 minutes then dropwise charged with TMS-Cl (121 mg, 1.1 mmol) and stirred at room temperature for 6 h. This reaction was charged with EDCI (35 mg, 0.18 mmol), and DMAP (22 mg, 0.18 mmol) and stirred at room temperature for 10 minutes then charged with N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (60 mg, 0.12 mmol) resulting in 25 mg, 32.7% yield of the title compound as an off white solid. ¹H NMR (400 MHz, Methanol-d₄) δ 7.70 (d, J=8.8 Hz, 1H), 7.54 (d, J=8.2 Hz, 2H), 7.45-7.33 (m, 3H), 7.21 (d, J=8.1 Hz, 1H), 6.91 (dd, J=9.6, 5.5 Hz, 2H), 6.67 (dd, J=9.7, 6.3 Hz, 1H), 4.62 (dd, J=9.3, 5.5 Hz, 1H), 3.82-3.38 (m, 4H), 3.49-3.32 (m, 2H), 2.63 (s, 3H), 1.64-1.47 (m, 4H), 1.29-0.120 (m, 2H). Mol. Wt: 617.09; MS (ES+): m/z 617.60 [MH⁺], HPLC purity: 96.51% (Max plot).

### Synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-4-(hydroxydimethylsilyl)benzamide (BRD-S-01)

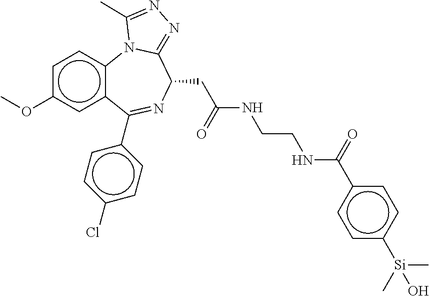

A solution of 4-(hydroxydimethylsilyl)benzoic acid (5 mg, 0.025 mmol) in DMF (2.5 mL) was charged with EDCI (6 mg, 0.033 mmol), DIPEA (6 mg, 0.044 mmol), and HOBt (4 mg, 0.033 mmol) and stirred at room temperature for 10 minutes. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (10 mg, 0.22 mmol) and stirred overnight at room temperature. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 3.46 mg, 24.02% yield of the corresponding title compound as a white solid. ¹H NMR (400 MHz, Methanol-d₄) δ 7.84-7.57 (m, 5H), 7.48 (dd, J=8.3, 5.1 Hz, 2H), 7.36-7.00 (m, 3H), 6.89-6.80 (m, 1H), 4.69-4.57 (m, 1H), 3.82 (s, 3H), 3.54 (dt, J=17.2, 6.4 Hz, 2H), 3.37-3.25 (m, 2H), 2.62 (s, 3H), 1.29-120 (m, 2H), 0.36 (s, 6H). Mol. Wt: 617.17; MS (ES+): m/z 617.20 [MH⁺], HPLC purity: 91.06 at Max plot.

The following compounds have been synthesized and purified using the general procedure described above for the synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-4-(hydroxydimethylsilyl)benzamide.

### Synthesis of N-(3-(2-((4S)-6-(4-chlorophenyl)-8-methoxy-1-methyl-4H-benzo[f][1,2,4]triazolo[4,3-a][1,4]diazepin-4-yl)acetamido)propyl)-4-(hydroxydimethylsilyl)benzamide (BRD-S-03)

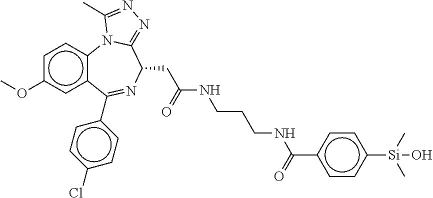

A solution of 4-(hydroxydimethylsilyl)benzoic acid (24 mg, 0.12 mmol) in DMF (2.5 mL) was charged with EDCI (32 mg, 0.16 mmol), DIPEA (28 mg, 0.22 mmol), and HOBt (12 mg, 0.16 mmol) and stirred at room temperature for 10 minutes. The solution was charged with N-(3-aminopropyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) resulting in 2.92 mg, 4.2% yield of the title compound as off white solid. ¹H NMR (400 MHz, Methanol-d₄) δ 7.82-7.49 (m, 6H), 7.46-7.25 (m, 4H), 6.91 (dd, J=7.7, 3.3 Hz, 1H), 4.69-4.60 (m, 1H), 3.84 (s, 3H), 3.49-3.34 (m, 3H), 2.63 (s, 3H), 1.87-1.54 (m, 3H), 1.30-1.66 (m, 2H), 0.37 (s, 6H). Mol. Wt: monomer: 631.20; MS (ES+): m/z 631.25 [MH⁺], Mol. Wt: dimer: 1244.4; 623.10 (MH⁺/2), HPLC purity: monomer: 52.59%, dimer: 23.13% at Max plot.

### Synthesis of N-(4-(2-((4S)-6-(4-chlorophenyl)-8-methoxy-1-methyl-4H-benzo[f][1,2,4]triazolo[4,3-a][1,4]diazepin-4-yl)acetamido)butyl)-4-(hydroxydimethylsilyl)benzamide (BRD-S-05)

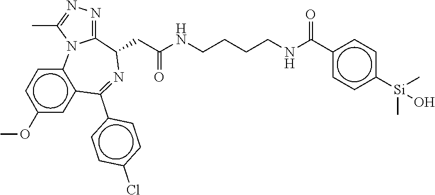

A solution of 4-(hydroxydimethylsilyl)benzoic acid (5 mg, 0.023 mmol) in DMF (2.5 mL) was charged with EDCI (6 mg, 0.031 mmol), DIPEA (5 mg, 0.04 mmol), and HOBt (4 mg, 0.031 mmol) and stirred at rt for 10 minutes and charged with N-(4-aminobutyl)-2-((4S)-6-(4-chlorophenyl)-8-methoxy-1-methyl-4H-benzo[f][1,2,4]triazolo[4,3-a][1,4]diazepin-4-yl)acetamide (10 mg, 0.021 mmol) resulting in 4.09 mg, 29.06% yield of the title compound as off white solid. ¹H NMR (400 MHz, Methanol-d₄) δ 8.49 (s, 1H), 8.39 (s, 1H), 7.84-7.48 (m, 7H), 7.37-7.10 (m, 3H), 6.90 (dd, J=6.6, 3.0 Hz, 1H), 4.62 (dd, J=8.8, 4.3 Hz, 1H), 3.81 (s, 3H), 3.48-3.32 (m, 3H), 3.25 (dd, J=14.8, 5.3 Hz, 1H), 2.62 (s, 3H), 1.75-1.61 (m, 4H), 1.30-1.23 (m, 3H), 0.35 (s, 6H). Mol. Wt: Monomer: 645.22, dimer: 1272.4; MS (ES+): m/z 645.3 [MH⁺], 637.10 [MH⁺/2], HPLC purity: monomer: 51.16%, dimer: 35.17% at Max plot.

### Synthesis of N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)-4-(hydroxydimethylsilyl)benzamide (BRD-S-07)

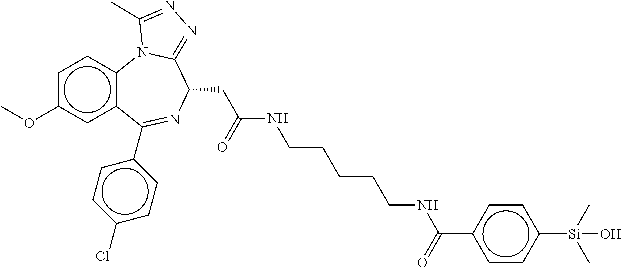

A solution of 4-(hydroxydimethylsilyl)benzoic acid (22 mg, 0.11 mmol) in DMF (2.5 mL) was charged with EDCI (29 mg, 0.15 mmol), DIPEA (39 mg, 0.3 mmol), and HOBt (20 mg, 0.15 mmol) and stirred at rt for 10 minutes then charged with N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.10 mmol) resulting in 4.08 mg, 6% yield of the title compound as off white solid. ¹H NMR (400 MHz, Methanol-d₄) δ 7.82-7.60 (m, 5H), 7.54 (d, J=8.4 Hz, 2H), 7.44-7.32 (m, 3H), 6.91 (d, J=2.9 Hz, 1H), 4.66-4.55 (m, 1H), 3.82 (s, 3H), 3.46-3.34 (m, 3H), 3.33-3.19 (m, 3H), 2.63 (s, 3H), 1.65-1.59 (m, 4H), 1.57-1.41 (m, 2H), 0.34 (s, 6H). Mol. Wt: monomer: 659.25, dimer: 1300.4, MS (ES+): m/z 659.2 [MH⁺], dimer 650.65 [MH⁺/2], HPLC purity: monomer: 81.03%, dimer: 4.80% at Max plot.

### Synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-3-(hydroxydimethylsilyl)benzamide (BRD-S-02)

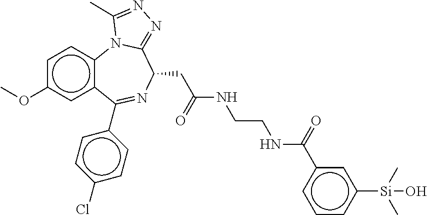

A solution of 3-(hydroxydimethylsilyl)benzoic acid (20 mg, 0.13 mmol) in DCM (5 mL) was charged with EDCI (32 mg, 0.17 mmol) and DMAP (6 mg, 0.05 mmol) and stirred at room temperature for 10 minutes. This solution was charged with N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.11 mmol) and stirred overnight at room temperature. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 10 mg, 14.2% yield of the title compound as a white solid. ¹H NMR (400 MHz, Methanol-d₄) δ 7.97-7.71 (m, 4H), 7.52-7.41 (m, 2H), 7.44-7.28 (m, 4H), 6.79 (d, J=2.7 Hz, 1H), 4.67 (dd, J=9.0, 5.5 Hz, 1H), 3.81 (s, 3H), 3.60-3.51 (m, 3H), 3.52-3.37 (m, 1H), 3.28 (d, J=9.3 Hz, 2H), 2.62 (s, 3H), 0.36 (s, 6H). Mol. Wt: monomer: 617.17, dimer: 1216.34, MS (ES+): m/z 617.60 [MH⁺], dimer 1216.34 [MH⁺] 609.75 [MH⁺/2], HPLC purity: monomer: 14.46%, dimer: 80.73% at Max plot.

The following compounds have been synthesized and purified using the general procedure described above for the synthesis of N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-3-(hydroxydimethylsilyl)benzamide.

### Synthesis of N-(3-(2-((4S)-6-(4-chlorophenyl)-8-methoxy-1-methyl-4H-benzo[f][1,2,4]triazolo[4,3-a][1,4]diazepin-4-yflacetarnido)propyl)-3-(hydroxydirnethylsilyflbenzarnide (BRD-S-04)

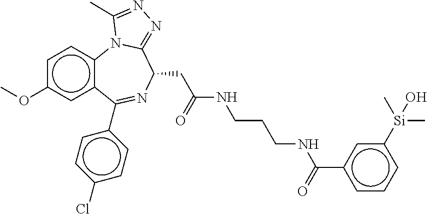

A solution of 3-(hydroxydimethylsilyl)benzoic acid (30 mg, 0.15 mmol) in DCM (5 mL) was charged with EDCI (38 mg, 0.19 mmol), DMAP (8 mg, 0.06 mmol), and stirred at rt for 10 minutes. This solution was charged with N-(3-aminoproyl)-2-((4S)-6-(4-chlorophenyl)-8-methoxy-1-methyl-4H-benzo[f][1,2,4]triazolo[4,3-a][1,4]diazepin-4-yl)acetamide (60 mg, 0.13 mmol) resulting in 8 mg, 9.6% yield of the title compound as white solid. ¹H NMR (400 MHz, Methanol-d₄) δ 7.97 (s, 1H), 7.88-7.62 (m, 3H), 7.59-7.50 (m, 2H), 7.38-7.00 (m, 4H), 6.92 (dd, J=14.9, 2.9 Hz, 1H), 4.70 (dd, J=8.3, 5.5 Hz, 1H), 3.81 (s, 3H), 3.51-3.32 (m, 4H), 2.70 (s, 3H), 1.85-160 (m, 2H), 1.33-1.55 (m, 2H), 0.35 (s, 6H). Mol. Wt: monomer: 631.20, dimer 1214.44, MS (ES+): m/z 631.50 [MH¹], 623.35 (MH⁺/2), HPLC purity: monomer: 22.21%, dimer: 69.66% at 220 nm.

### Synthesis of N-(4-(2-((4S)-6-(4-chlorophenyl)-8-methoxy-1-methyl-4H-benzo[f][1,2,4]triazolo[4,3-a][1,4]diazepin-4-yflacetarnido)butyl)-3-(hydroxydimethylsilyflbenzamide (BRD-S-06)

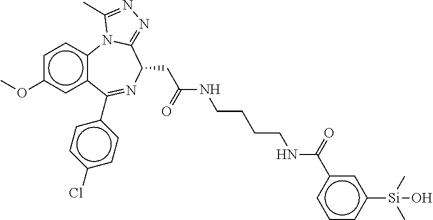

A solution of 3-(hydroxydimethylsilyl)benzoic acid (25 mg, 0.12 mmol) in DCM (5 mL) was charged with EDCI (30 mg, 0.16 mmol), DMAP (6.5 mg, 0.05 mmol), and stirred at rt for 10 minutes. This solution was charged with N-(4-aminobutyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.10 mmol) resulting in 10 mg, 14.7% yield of the title compound as white solid. ¹H NMR (400 MHz, Methanol-d₄) δ 7.85-7.65 (m, 3H), 7.56-7.31 (m, 7H), 6.88 (dd, J=7.6, 2.9 Hz, 1H), 4.67-4.58 (m, 1H), 3.81 (s, 3H), 3.47-3.31 (m, 3H), 3.31-3.20 (m, 3H), 2.62 (s, 3H), 1.75-1.59 (m, 4H), 0.35 (s, 6H). Mol. Wt: monomer: 645.22, dimer: 1272.44, MS (ES+): m/z 645.55 [MH⁺], dimer: 637.40 (MH⁺/2). HPLC purity: monomer: 91.9%, dimer: 3.45% at 254 nm.

### Synthesis of N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)-3-(hydroxydimethylsilyl)benzamide (BRD-S-08)

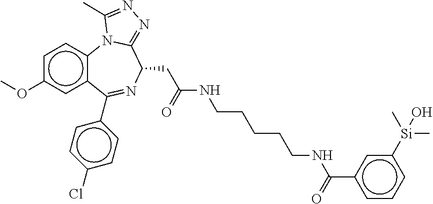

A solution of 3-(hydroxydimethylsilyl)benzoic acid (24.3 mg, 0.12 mmol) in DCM (5 mL) was charged with EDCI (29 mg, 0.15 mmol), DMAP (6.3 mg, 0.05 mmol), and stirred at rt for 10 minutes. This solution was charged with N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (50 mg, 0.10 mmol) resulting in 15 mg, 22% yield of the title compound as white solid. ¹H NMR (400 MHz, Methanol-d₄) δ 8.04 (s, 1H), 7.84-7.66 (m, 3H), 7.58-7.49 (m, 3H), 7.48-7.31 (m, 4H), 6.91 (q, J=4.1, 3.0 Hz, 1H), 4.62 (dd, J=8.4, 5.0 Hz, 1H), 3.82 (s, 3H), 3.46-3.17 (m, 7H), 2.63 (s, 3H), 1.74-1.41 (m, 5H), 0.35 (s, 6H). Mol. Wt: monomer: 659.25, dimer: 1300.25; MS (ES+): m/z 659.20 [MH⁺], dimer: 651.40 (MH⁺/2), HPLC purity: monomer: 95.94%, dimer: 2.90% at 254 nm.

### Synthesis of ((S)—N-(2-aminoethyl)-2-(6-(4-chlorophenyl)-8-methoxy-1-methyl-4H-benzo[f][1,2,4]triazolo[4,3-a][1,4]diazepin-4-yl)acetamide (BRD-C-34)

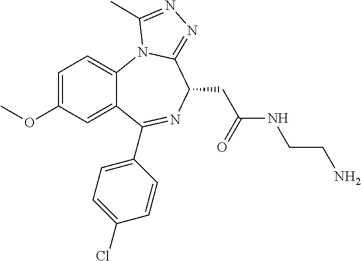 [SMILES: COc1ccc2c(c1)C(c1ccc(Cl)cc1)=N[C@@H](CC(=O)NCCN)c1nnc(C)n1-2]

A solution (S)-tert-butyl(2-(2-(6-(4-chlorophenyl)-8-methoxy-1-methyl-4H-benzo[f][1,2,4]triazolo[4,3-a][1,4]diazepin-4-yl)acetamido)ethyl)carbamate (2.8 g, 0.52 mmol) in DCM (50 mL) was charged with TFA (5 mL) and stirred for at room temperatrue for 12 h. The reaction mixture was then evaporated under reduced pressure to obtain a residue which was redissolved in DCM (10 mL) and charged with powdered KOH to adjust the pH to −8-9 then filtered through a pad of celite. The resulting filtrate was concentrated in vacuo resulting in a crude product which was purified by column chromatography on silica gel (100-200 mesh), eluting with 5% methanol in chloroform resulting in 1.7 g, 74.59% yield of the title compound as a yellow solid. ¹H NMR (400 MHz, CDCl₃) δ 7.59 (t, J=5.9 Hz, 1H), 7.54-7.46 (m, 2H), 7.43-7.30 (m, 2H), 7.20 (dd, J=9.0, 2.9 Hz, 1H), 6.86 (d, J=2.8 Hz, 1H), 4.65 (dd, J=8.4, 6.1 Hz, 1H), 3.80 (s, 3H), 3.62-3.45 (m, 2H), 3.33 (dt, J=13.3, 6.7 Hz, 2H), 3.01-2.86 (m, 2H), 2.60 (s, 3H). Mol Wt:—438.09, MS (ES+): m/z 439.15 [MH¹], HPLC purity:—98.92% (254 nm).

The following compounds have been synthesized and purified using the general procedure described above for the synthesis of ((S)—N-(2-aminoethyl)-2-(6-(4-chlorophenyl)-8-methoxy-1-methyl-4H-benzo[f][1,2,4]triazolo[4,3-a][1,4]diazepin-4-yl)acetamide.

### Synthesis of (S)—N-(3-aminopropyl)-2-(6-(4-chlorophenyl)-8-methoxy-1-methyl-4H-benzo[f][1,2,4]triazolo[4,3-a][1,4]diazepin-4-yl)acetamide (BRD-C-35)

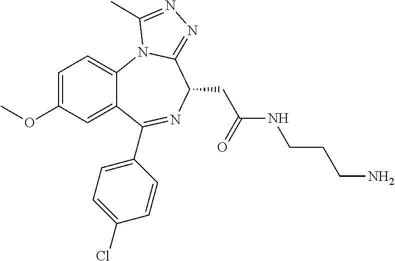 [SMILES: COc1ccc2c(c1)C(c1ccc(Cl)cc1)=N[C@@H](CC(=O)NCCCN)c1nnc(C)n1-2]

A solution of (S)-tert-butyl(3-(2-(6-(4-chlorophenyl)-8-methoxy-1-methyl-4H-benzo[f][1,2,4]triazolo[4,3-a][1,4]diazepin-4-yl)acetamido)propyl)carbamate (1.0 g, 20 mmol) in DCM (20 mL) was charged with TFA (10 mL) resulting in 700 mg, 77.7% yield of the title compound as a yellow solid. ¹H NMR (400 MHz, DMSO-d₆) δ 8.51 (s, 1H), 8.12 (s, 2H), 7.87 (d, J=8.4 Hz, 1H), 7.57-7.39 (m, 5H), 6.89 (d, J=2.9 Hz, 1H), 4.61-4.53 (m, 1H), 3.81 (s, 3H), 3.52-3.35 (m, 1H), 3.35-3.11 (m, 5H), 2.90-2.79 (m, 2H), 2.67 (s, 3H). Mol Wt: 452.94, MS (ES+): m/z 453.15 [MH¹], HPLC purity: 96.37% (220 nm).

### Synthesis of (S)—N-(4-aminobutyl)-2-(6-(4-chlorophenyl)-8-methoxy-1-methyl-4H-benzo[f][1,2,4]triazolo[4,3-a][1,4]diazepin-4-yl)acetamide (BRD-C-36)

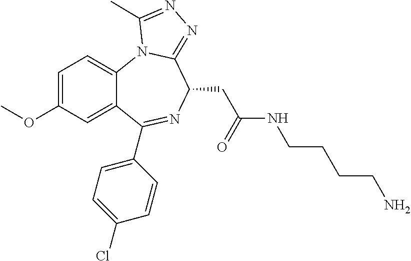 [SMILES: COc1ccc2c(c1)C(c1ccc(Cl)cc1)=N[C@@H](CC(=O)NCCCCN)c1nnc(C)n1-2]

A solution of (S)-tert-butyl(4-(2-(6-(4-chlorophenyl)-8-methoxy-1-methyl-4H-benzo[f][1,2,4]triazolo[4,3-a][1,4]diazepin-4-yl)acetamido)butyl)carbamate (1.1 g, 0.19 mmol) in DCM (25 mL) was charged with TFA (2.5 mL) resulting in 550 mg, 60.77% yield of the title compound as a yellow solid. ¹H NMR (400 MHz, CDCl₃) δ 7.53-7.45 (m, 2H), 7.42-7.29 (m, 3H), 7.19 (dd, J=8.9, 2.9 Hz, 1H), 6.88 (d, J=2.9 Hz, 1H), 4.61 (t, J=7.1 Hz, 1H), 3.80 (s, 3H), 3.52 (dd, J=14.1, 7.6 Hz, 1H), 3.34-3.21 (m, 4H), 2.72 (t, J=6.7 Hz, 2H), 2.61 (s, 3H), 1.64-1.53 (m, 1H), 1.55-1.43 (m, 2H). Mol Wt: 466.96, MS (ES+): m/z 467.35 [MH⁺], HPLC purity: 99.09% (254 nm).

### Synthesis of (S)—N-(5-aminopentyl)-2-(6-(4-chlorophenyl)-8-methoxy-1-methyl-4H-benzo[f][1,2,4]triazolo[4,3-a][1,4]diazepin-4-yl)acetamide (BRD-C-37)

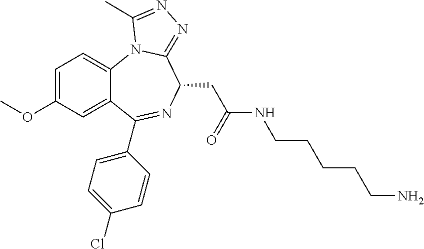 [SMILES: COc1ccc2c(c1)C(c1ccc(Cl)cc1)=N[C@@H](CC(=O)NCCCCCN)c1nnc(C)n1-2]

A solution of (S)-tert-butyl(5-(2-(6-(4-chlorophenyl)-8-methoxy-1-methyl-4H-benzo[f][1,2,4]triazolo[4,3-a][1,4]diazepin-4-acetamido)pentyl)carbamate (1.2 g, 0.20 mmol) in DCM (50 mL) was charged with TFA (2.5 mL) resulting in 600 mg, 60.42% yield of the title compound as a yellow solid. ¹H NMR (400 MHz, CDCl₃) δ 7.53-7.43 (m, 2H), 7.42-7.29 (m, 2H), 7.20 (dd, J=8.9, 2.9 Hz, 1H), 6.85 (d, J=2.9 Hz, 1H), 6.76 (t, J=5.8 Hz, 1H), 4.62 (dd, J=7.7, 6.4 Hz, 1H), 3.80 (s, 3H), 3.55-3.48 (m, 2H), 3.35-3.20 (m, 3H), 2.70 (t, J=6.7 Hz, 2H), 2.61 (s, 3H), 1.62-1.31 (m, 3H), 1.33-1.23 (m, 2H). Mol Wt: 480.99, MS (ES+): m/z 481.25 [MH⁺], HPLC purity: 99.02% (254 nm).

### Synthesis of (S)-tert-butyl(2-(2-(6-(4-chlorophenyl)-8-methoxy-1-methyl-4H-benzo[f][1,2,4]triazolo[4,3-a][1,4]diazepin-4-yl)acetamido)ethyl)carbamate (BRD-C-01)

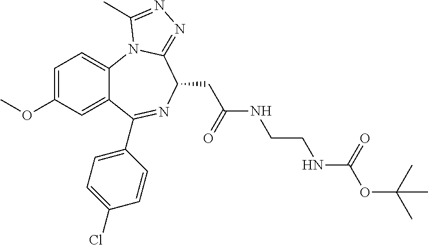 [SMILES: COc1ccc2c(c1)C(c1ccc(Cl)cc1)=N[C@@H](CC(=O)NCCNC(=O)OC(C)(C)C)c1nnc(C)n1-2]

A suspension of (S)-2-(6-(4-chlorophenyl)-8-methoxy-1-methyl-4H-benzo[f][1,2,4]triazolo[4,3-a][1,4]diazepin-4-yl)acetic acid (2.5 g, 6.3 mmol) in DCM (6 mL) was charged with EDCI (1.79 g, 9.4 mmol), 4-DMAP (1.1 g, 9.4 mmol), and HoBt (1.2 g, 9.4 mmol) and stirred at room temperaturet for 10 minutes. This solution was charged with tert-butyl(2-aminoethyl) carbamate (1.50 g, 9.4 mmol) and stirred overnight at room temperature. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by column chromatography on silica gel (100-200 mesh), eluting with 2% methanol in chloroform to afford 3.30 g, 85.5% of the title compound as an off white solid. ¹H NMR (400 MHz, CDCl₃) δ 7.53-7.45 (m, 2H), 7.42-7.29 (m, 2H), 7.20 (dd, J=8.9, 2.9 Hz, 1H), 7.03 (d, J=7.3 Hz, 1H), 6.86 (d, J=2.9 Hz, 1H), 4.63 (t, J=7.0 Hz, 1H), 3.80 (s, 3H), 3.51 (dd, J=14.5, 7.3 Hz, 1H), 3.45-3.21 (m, 5H), 2.62 (s, 3H), 1.44 (s, 9H). Mol Wt:—539.03, MS (ES+): m/z 539.15 [MH⁺], HPLC purity: 99.63% (254 nm).

The following compounds have been synthesized and purified using the general procedure described above for the synthesis of (S)-tert-butyl(2-(2-(6-(4-chlorophenyl)-8-methoxy-1-methyl-4H-benzo[f][1,2,4]triazolo[4,3-a][1,4]diazepin-4-yl)acetamido)ethyl)carbamate.

### Synthesis of (S)-tert-butyl(3-(2-(6-(4-chlorophenyl)-8-methoxy-1-methyl-4H-benzo[f][1,2,4]triazolo[4,3-a][1,4]diazepin-4-yl)acetamido)propyl)carbamate (BRD-C-04)

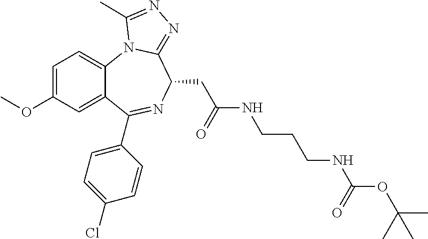 [SMILES: COc1ccc2c(c1)C(c1ccc(Cl)cc1)=N[C@@H](CC(=O)NCCCNC(=O)OC(C)(C)C)c1nnc(C)n1-2]

A suspension of (S)-2-(6-(4-chlorophenyl)-8-methoxy-1-methyl-4H-benzo[f][1,2,4]triazolo[4,3-a][1,4]diazepin-4-yl)acetic acid (50 mg, 0.12 mmol) in DCM (6 mL) was charged with EDCI (34.4 mg, 0.18 mmol), 4-DMAP (21.9 mg, 0.18 mmol), and HoBt (24.3 mg, 0.18 mmol) and stirred at room temperatrue for 10 minutes. This solution was charged with tert-butyl(3-aminopropyl) carbamate (32.95 mg, 0.18 mmol) resulting in 280 mg, 80.0% yield of the title compound as colorless oil. ¹H NMR (400 MHz, CDCl₃) δ 7.48 (d, J=8.4 Hz, 2H), 7.40-7.28 (m, 2H), 7.20 (dd, J=9.0, 2.9 Hz, 1H), 6.96 (t, J=6.3 Hz, 1H), 6.86 (d, J=2.9 Hz, 1H), 4.63 (t, J=7.0 Hz, 1H), 3.80 (s, 3H), 3.50 (dd, J=14.4, 7.1 Hz, 1H), 3.44-3.27 (m, 5H), 3.15 (q, J=6.4 Hz, 2H), 2.62 (s, 3H), 1.43 (s, 9H). Mol Wt: 553.05, MS (ES+): m/z 553.35 [MH⁺], HPLC purity: 99.57% (254 nm).

### Synthesis of (S)-tert-butyl(4-(2-(6-(4-chlorophenyl)-8-methoxy-1-methyl-4H-benzo[f][1,2,4]triazolo[4,3-a][1,4]diazepin-4-yl)acetamido)butyl)carbamate (BRD-C-07)

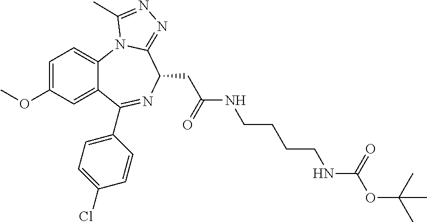 [SMILES: COc1ccc2c(c1)C(c1ccc(Cl)cc1)=N[C@@H](CC(=O)NCCCCNC(=O)OC(C)(C)C)c1nnc(C)n1-2]

A suspension of (S)-2-(6-(4-chlorophenyl)-8-methoxy-1-methyl-4H-benzo[f][1,2,4]triazolo[4,3-a][1,4]diazepin-4-yl)acetic acid (1.0 g, 0.25 mmol) in DCM (6 mL) was charged with EDCI (0.716 g, 0.37 mmol), 4-DMAP (0.457 mg, 0.37 mmol), and HoBt (0.506 g, 0.37 mmol) and stirred at room temperature for 10 minutes. This solution was charged with tert-butyl(4-aminobutyl) carbamate (0.712 g, 0.37 mmol) resulting in 1.1 g, 78.5% yield of the title compound as white solid. ¹H NMR (400 MHz, CDCl₃) δ 7.49 (d, J=8.5 Hz, 2H), 7.43-7.31 (m, 2H), 7.20 (dd, J=9.0, 2.9 Hz, 1H), 6.86 (d, J=2.9 Hz, 1H), 6.61 (t, J=6.0 Hz, 1H), 4.60 (t, J=7.1 Hz, 1H), 3.80 (s, 3H), 3.53 (dd, J=14.2, 7.8 Hz, 1H), 3.30 (dt, J=13.8, 7.0 Hz, 4H), 3.12 (d, J=8.5 Hz, 3H), 2.61 (s, 3H), 1.60-1.47 (m, 2H), 1.44 (s, 9H). Mol Wt: 567.08, MS (ES+): m/z 567.35 [MH⁺], HPLC purity: 99.77% (254 nm).

### Synthesis of (S)-tert-butyl(5-(2-(6-(4-chlorophenyl)-8-methoxy-1-methyl-4H-benzo[f][1,2,4]triazolo[4,3-a][1,4]diazepin-4-acetamido)pentyl)carbamate (BRD-C-10)

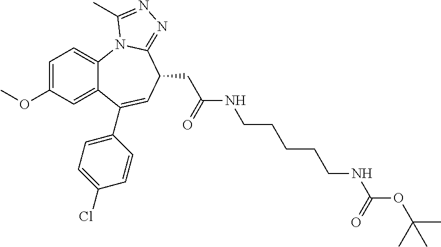 [SMILES: COc1ccc2c(c1)C(c1ccc(Cl)cc1)=C[C@@H](CC(=O)NCCCCCNC(=O)OC(C)(C)C)c1nnc(C)n1-2]

A suspension of (S)-2-(6-(4-chlorophenyl)-8-methoxy-1-methyl-4H-benzo[f][1,2,4]triazolo[4,3-a][1,4]diazepin-4-yl)acetic acid (1 g, 0.25 mmol) in DCM (6 mL) were charged with EDCI (0.706 g, 0.37 mmol), 4-DMAP (0.451 g, 0.37 mmol), and HoBt (0.499 g, 0.37 mmol) and stirred at room temperature for 10 minutes. This solution was charged with tert-butyl(5-aminopentyl) carbamate (0.765 g, 0.37 mmol) resulting in 1.2 g, 82.1% yield of the title compound as a white solid. ¹H NMR (400 MHz, CDCl₃) δ 7.44 (dd, J=8.6, 2.9 Hz, 2H), 7.37-7.26 (m, 3H), 7.23-7.16 (m, 1H), 6.88-6.82 (m, 1H), 4.59 (t, J=7.1 Hz, 1H), 3.79 (s, 3H), 3.53 (dd, J=14.0, 7.9 Hz, 1H), 3.36-3.18 (m, 3H), 3.07 (q, J=7.5 Hz, 2H), 2.61 (s, 3H), 1.71 (s, 9H), 1.62-1.45 (m, 4H), 1.39-1.22 (m, 3H). Mol Wt: 581, MS (ES+): m/z 581.35 [MH⁺], HPLC purity: 99.20% (254 nm).

### Example 357

Monomers were synthesized according to the procedures described below.

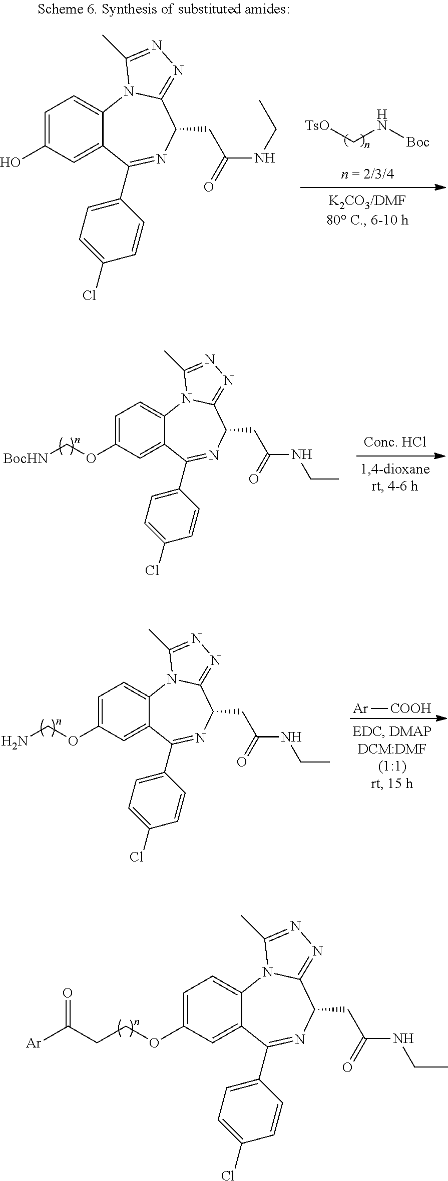

### Synthesis of (S)—N-(2-((6-(4-chlorophenyl)-4-(2-(ethylamino)-2-oxoethyl)-1-methyl-4H-benzo[f][1,2,4]triazolo[4,3-a]diazepin-8-yl)oxy)ethyl)benzamide (BRD-C-28)

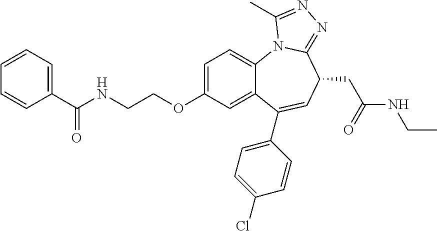 [SMILES: CCNC(=O)C[C@@H]1C=C(c2ccc(Cl)cc2)c2cc(OCCNC(=O)c3ccccc3)ccc2-n2c(C)nnc21]

General Procedure:

A solution of benzoic acid (18 mg, 0.154 mmol) in DCM:DMF (15 mL/g) was charged with EDCI (44 mg, 0.231 mmol) and stirred at room temperature for 10 minutes. This solution was charged with (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde (70 mg, 0.154 mmol) and DMAP (28 mg, 0.231 mmol) and stirred at room temperature overnight. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to resulting in 29 mg, 34% yield of the title compound as a white solid. ¹H NMR (400 MHz, CD₃OD): 7.81 (d, J=8.0 Hz, 2H), 7.75 (d, J=8.8 Hz, 1H), 7.58-7.51 (m, 3H), 7.49-7.43 (m, 3H), 7.39 (d, J=8.0 Hz, 2H), 7.02 (d, J=2.8 Hz, 1H), 4.67 (dd, J=8.0, 5.6 Hz, 1H), 4.30-4.16 (m, 2H), 3.82-3.72 (m, 2H), 3.44-3.35 (m, 1H), 3.28-3.19 (m, 3H), 2.72 (s, 3H), 1.18 (t, J=7.2 Hz, 3H). Mol. Wt: 557.04; MS (ES+): m/z 557.20[MH⁺].

The following compounds have been synthesized and purified using the general procedure described above for the synthesis of (S)—N-(2-((6-(4-chlorophenyl)-4-(2-(ethylamino)-2-oxoethyl)-1-methyl-4H-benzo[f][1,2,4]triazolo[4,3-a]diazepin-8-yl)oxy)ethyl)benzamide.

### Synthesis of (S)-(3-((2-((6-(4-chlorophenyl)-4-(2-(ethylamino)-2-oxoethyl)-1-methyl-4H-benzo[f][1,2,4]triazolo[4,3-a]diazepin-8-yl)oxy)ethyl)carbamoyl)phenyl)boronic acid (BRD-E-21)

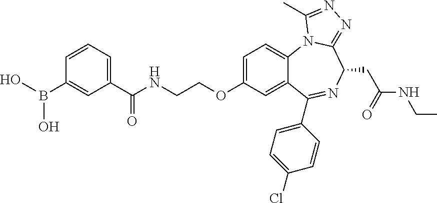 [SMILES: CCNC(=O)C[C@@H]1N=C(c2ccc(Cl)cc2)c2cc(OCCNC(=O)c3cccc(B(O)O)c3)ccc2-n2c(C)nnc21]

A solution of 3-boronobenzoic acid (22 mg, 0.132 mmol) in DCM:DMF (15 mL/g) was charged with EDCI (37 mg, 0.198 mmol) and stirred at rt for 10 minutes. This solution was charged with (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde (60 mg, 0.132 mmol) and DMAP (24 mg, 0.198 mmol) resulting in 15 mg, 19% yield of the title compound as a white solid. ¹H NMR (400 MHz, CD₃OD): δ 8.05 (s, 1H), 7.85-7.76 (m, 2H), 7.73 (d, J=8.8 Hz, 1H), 7.54 (d, J=8.2 Hz, 2H), 7.50-7.42 (m, 2H), 7.39 (d, J=8.2 Hz, 2H), 7.00 (s, 1H), 4.65 (dd, J=8.4, 5.6 Hz, 1H), 4.30-4.15 (m, 2H), 3.82-3.70 (m, 2H), 3.44-3.36 (m, 2H), 3.29-3.20 (m, 3H), 2.68 (s, 3H), 1.18 (t, J=7.2 Hz, 3H). Mol. Wt: 600.86; MS (ES+): m/z 601.20 [MH⁺].

### Synthesis of (S)-(4-((2-((6-(4-chlorophenyl)-4-(2-(ethylamino)-2-oxoethyl)-1-methyl-4H-benzo[f][1,2,4]triazolo[4,3-a]diazepin-8-yl)oxy)ethyl)carbamoyl)phenyl)boronic acid (BRD-E-22)

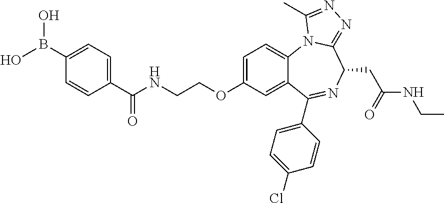 [SMILES: CCNC(=O)C[C@@H]1N=C(c2ccc(Cl)cc2)c2cc(OCCNC(=O)c3ccc(B(O)O)cc3)ccc2-n2c(C)nnc21]

A solution of 4-boronobenzoic acid (25 mg, 0.154 mmol) in DCM:DMF (15 mL/g) was charged with EDCI (44 mg, 0.232 mmol) and stirred at room temperature for 10 minutes. This solution was charged with (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde (70 mg, 0.154 mmol) and DMAP (28 mg, 0.232 mmol) resulting in 15 mg, 16% yield of the title compound as a white solid. ¹H NMR (400 MHz, CD₃OD): −δ 7.80-7.67 (m, 5H), 7.54 (d, J=8.6 Hz, 2H), 7.46 (dd, J=8.8, 2.8 Hz, 1H), 7.39 (d, J=8.6 Hz, 2H), 7.01 (d, J=2.8 Hz, 1H), 4.66 (dd, J=8.40, 5.6 Hz, 1H), 4.30-4.18 (m, 2H), 3.77 (t, J=5.6 Hz, 2H), 3.43-3.35 (m, 1H), 3.30-3.20 (m, 3H), 2.70 (s, 3H), 1.18 (t, J=7.2 Hz, 3H). Mol. Wt: 600.86; MS (ES+): m/z 601.20 [MH⁺].

### Synthesis of (S)—N-(2-((6-(4-chlorophenyl)-4-(2-(ethylamino)-2-oxoethyl)-1-methyl-4H-benzo[f][1,2,4]triazolo[4,3-a]diazepin-8-yl)oxy)ethyl-3,4-dihydroxybenzamide (BRD-N-24)

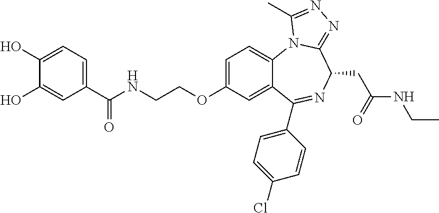 [SMILES: CCNC(=O)C[C@@H]1N=C(c2ccc(Cl)cc2)c2cc(OCCNC(=O)c3ccc(O)c(O)c3)ccc2-n2c(C)nnc21]

A solution of 3,4-dihydroxybenzoic acid (32 mg, 0.20 mmol) in DCM:DMF (15 mL/g) was charged with EDCI (48 mg, 0.25 mmol) and stirred at room temperature for 10 minutes. This solution was charged with (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde (80 mg, 0.176 mmol) and HOBt (32 mg, 0.24 mmol), DIPEA (45 mg, 0.35 mmol) resulting in 90 mg, 77% yield of the title compound as a white solid. ¹H NMR (400 MHz, CD₃OD): δ 7.71 (d, J=8.8 Hz, 1H), 7.53 (d, J=8.0 Hz, 2H), 7.46-7.36 (m, 3H), 7.25 (s, 1H), 7.17 (d, J=8.4, 1H), 6.97 (s, 1H), 6.78 (d, J=8.4 Hz, 1H), 4.63 (dd, J=8.0, 5.2 Hz, 1H), 4.26-4.12 (m, 2H), 3.76-3.64 (m, 2H), 3.44-3.35 (m, 1H), 3.20-3.09 (m, 1H), 2.67 (s, 3H), 1.18 (t, J=7.2 Hz, 3H). Mol. Wt: 589.04; MS (ES+): m/z 589.05 [MH⁺].

### Synthesis of (S)-(3-((3-((6-(4-chlorophenyl)-4-(2-(ethylamino)-2-oxoethyl)-1-methyl-4H-benzo[f][1,2,4]triazolo[4,3-a][1,4]diazepin-8-yl)oxy)propyl)carbamoyl)phenyl)boronic acid (BRD-E-23)

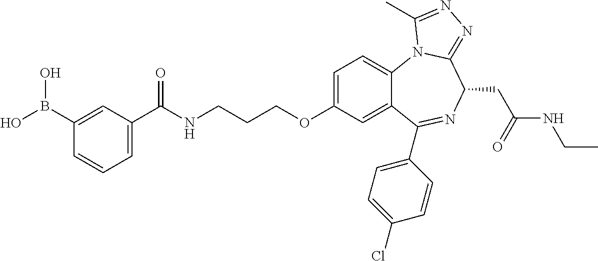 [SMILES: CCNC(=O)C[C@@H]1N=C(c2ccc(Cl)cc2)c2cc(OCCCNC(=O)c3cccc(B(O)O)c3)ccc2-n2c(C)nnc21]

A solution of 3-boronobenzoic acid (30 mg, 0.18 mmol) in DCM:DMF (15 mL/g) was charged with EDCI (51 mg, 0.27 mmol) and stirred at room temperature for 10 minutes. This solution was charged with (S)-2-(8-(3-aminopropoxy)-6-(4-chlorophenyl)-1-methyl-4H-benzo[f][1,2,4]triazolo[4,3-a][1,4]diazepin-4-yl)-N-ethylacetaminde (100 mg, 0.216 mmol) and DMAP (32 mg, 0.27 mmol) resulting in 11 mg, 9% yield of the title compound as an off white solid. ¹H NMR (400 MHz, CD₃OD): δ 8.05 (s, 1H), 7.85-7.68 (m, 3H), 7.53 (d, J=8.4 Hz, 2H), 7.49-7.30 (m, 4H), 6.94 (s, 1H), 4.72-4.60 (m, 1H), 4.20-4.02 (m, 2H), 3.64-3.50 (m, 2H), 2.70 (s, 3H), 2.16-2.04 (m, 2H), 1.84 (t, J=7.2 Hz, 3H). Mol. Wt: 614.89; MS (ES+): m/z 615.20 [MH⁺].

### Synthesis of (S)-(4-((3-((6-(4-chlorophenyl)-4-(2-(ethylamino)-2-oxoethyl)-1-methyl-4H-benzo[f][1,2,4]triazolo[4,3-a][1,4]diazepin-8-yl)oxy)propyl)carbamoyl)phenyl)-boronic acid (BRD-E-24)

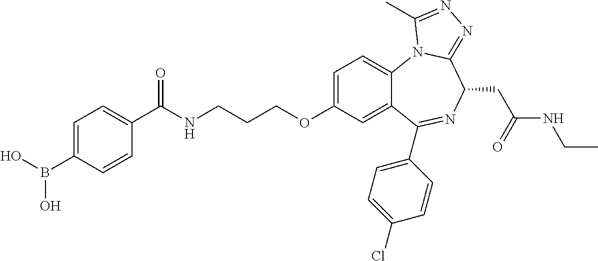 [SMILES: CCNC(=O)C[C@@H]1N=C(c2ccc(Cl)cc2)c2cc(OCCCNC(=O)c3ccc(B(O)O)cc3)ccc2-n2c(C)nnc21]

A solution of 4-boronobenzoic acid (30 mg, 0.18 mmol) in DCM:DMF (15 mL/g) was charged with EDCI (51 mg, 0.27 mmol) and stirred at room temperature for 10 minutes. This solution was charged with (S)-2-(8-(3-aminopropoxy)-6-(4-chlorophenyl)-1-methyl-4H-benzo[f][1,2,4]triazolo[4,3-a][1,4]diazepin-4-yl)-N-ethylacetaminde (100 mg, 0.216 mmol) and DMAP (32 mg, 0.27 mmol) resulting in 6.5 mg, 6% yield of the title compound as an off white solid. ¹H NMR (400 MHz, CD₃OD): δ 7.81-7.65 (m, 5H), 7.53 (d, J=8.4 Hz, 2H), 7.43-7.33 (m, 3H), 6.92 (s, 1H), 4.64 (dd, J=8.8, 5.6 Hz, 1H), 4.18-4.04 (m, 2H), 3.60-3.52 (m, 2H), 3.44-3.36 (m, 1H), 3.28-3.20 (m, 3H), 2.67 (s, 3H), 2.15-2.05 (m, 2H), 1.82 (t, J=7.2 Hz, 3H). Mol. Wt: 614.89; MS (ES+): m/z 615.20[MH⁺].

### Synthesis of (S)—N-(3-((6-(4-chlorophenyl)-4-(2-(ethylamino)-2-oxoethyl)-1-methyl-4H-benzo[f][1,2,4]triazolo[4,3-a][1,4]diazepin-8-yl)oxy)propyl)-3,4-dihydroxybenzaamide (BRD-N-26)

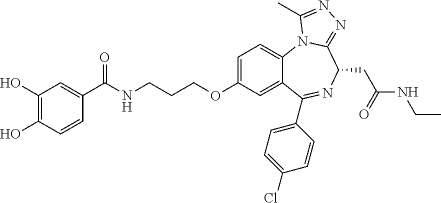 [SMILES: CCNC(=O)C[C@@H]1N=C(c2ccc(Cl)cc2)c2cc(OCCCNC(=O)c3ccc(O)c(O)c3)ccc2-n2c(C)nnc21]

A solution of 3,4-dihydroxybenzoic acid (33 mg, 0.214 mmol) in DCM:DMF (15 mL/g) was charged with EDCI (61 mg, 0.321 mmol) and stirred at room temperature for 10 minutes. This solution was charged with (S)-2-(8-(3-aminopropoxy)-6-(4-chlorophenyl)-1-methyl-4H-benzo[f][1,2,4]triazolo[4,3-a][1,4]diazepin-4-yl)-N-ethylacetaminde (100 mg, 0.214 mmol), HOBt (43 mg, 0.321 mmol), and DIPEA (0.1 ml, 0.535 mmol) resulting in 6.5 mg, 5% yield of the title compound as an off white solid. ¹H NMR (400 MHz, CD₃OD): δ 7.51 (d, J=8.4 Hz, 1H), 7.32-7.18 (m, 3H), 7.18-7.10 (m, 3H), 7.99 (d, J=8.4 Hz, 2H), 6.79 (d, J=8.4 Hz, 1H), 4.23 (t, J=6.0 Hz, 2H), 3.58 (t, J=6.4 Hz, 2H), 3.28-3.20 (m, 2H), 2.33 (s, 3H), 2.20-2.10 (m, 2H), 1.14 (t, J=7.2 Hz, 3H). Mol. Wt: 603.07; MS (ES+): m/z 603.20[MH⁺].

### Synthesis of (S)-(3-((4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butyl)carbamoyl)phenyl)boronic acid (BRD-E-25)

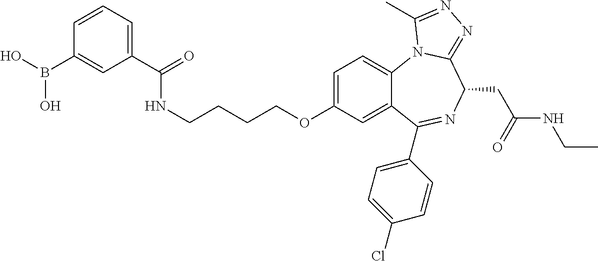 [SMILES: CCNC(=O)C[C@@H]1N=C(c2ccc(Cl)cc2)c2cc(OCCCCNC(=O)c3cccc(B(O)O)c3)ccc2-n2c(C)nnc21]

A solution of 3-boronobenzoic acid (30 mg, 0.18 mmol) in DCM:DMF (15 mL/g) was charged with EDCI (50 mg, 0.26 mmol) and stirred at room temperature for 10 minutes. This solution was charged with (S)-2-(8-(4-aminobutoxy)-6-(4-chlorophenyl)-1-methyl-4H-benzo[f][1,2,4]triazolo[4,3-a][1,4]diazepin-4-yl)-N-ethylacetaminde (100 mg, 0.21 mmol) and DMAP (32 mg, 0.26 mmol) resulting in 10 mg, 8% yield of the title compound as a white solid. ¹H NMR (400 MHz, CD₃OD): δ 8.05 (s, 1H), 7.86-7.74 (m, 1H), 7.70 (d, J=8.8 Hz, 1H), 7.54 (d, J=8.0 Hz, 2H), 7.50-7.34 (m, 4H), 6.93 (s, 1H), 4.68-4.60 (m, 1H), 4.13-4.00 (m, 2H), 3.52-3.35 (m, 4H), 3.28-3.20 (m, 2H), 2.67 (s, 3H), 1.90-1.74 (m, 4H), 1.80 (t, J=7.2 Hz, 3H. Mol. Wt: 628.91; MS (ES+): m/z 629.25[MH⁺].

### Synthesis of (S)-(4-((4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butyl)carbamoyl)phenyl)boronic acid (BRD-E-26)

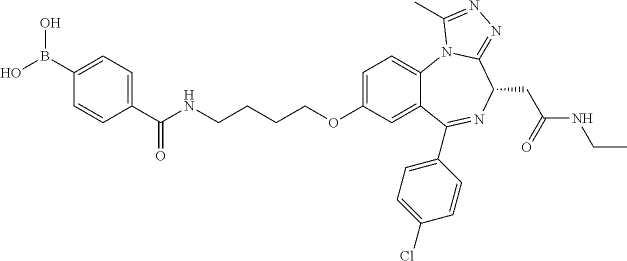 [SMILES: CCNC(=O)C[C@@H]1N=C(c2ccc(Cl)cc2)c2cc(OCCCCNC(=O)c3ccc(B(O)O)cc3)ccc2-n2c(C)nnc21]

A solution of 4-boronobenzoic acid (30 mg, 0.18 mmol) in DCM:DMF (15 mL/g) was charged with EDCI (50 mg, 0.26 mmol) and stirred at room temperature for 10 minutes. This solution was charged with (S)-2-(8-(4-aminobutoxy)-6-(4-chlorophenyl)-1-methyl-4H-benzo[f][1,2,4]triazolo[4,3-a][1,4]diazepin-4-yl)-N-ethylacetaminde (100 mg, 0.21 mmol) and DMAP (32 mg, 0.26 mmol) resulting in 13 mg, 10% yield of the title compound as a white solid. ¹H NMR (400 MHz, CD₃OD): δ 7.80-7.65 (m, 5H), 7.54 (d, J=8.4 Hz, 2H), 7.44-7.36 (m, 3H), 6.94 (d, J=2.4 Hz, 1H), 4.65 (dd, J=5.6, 2.8 Hz, 1H), 4.14-4.02 (m, 2H), 3.50-3.34 (m, 4H), 3.28-3.20 (m, 2H), 2.69 (s, 3H), 1.90-1.75 (m, 4H), 1.18 (t, J=7.2 Hz, 3H). Mol. Wt: 628.91; MS (ES+): m/z 629.20[MH⁺].

### Synthesis of (S)—N-(4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butyl)-3,4-dihydroxybenzamide (BRD-N-28)

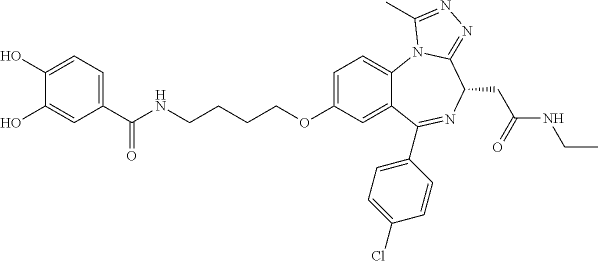 [SMILES: CCNC(=O)C[C@@H]1N=C(c2ccc(Cl)cc2)c2cc(OCCCCNC(=O)c3ccc(O)c(O)c3)ccc2-n2c(C)nnc21]

A solution of 3,4 dihydroxybenzoic acid (28 mg, 0.18 mmol) in DCM:DMF (15 mL/g) was charged with EDCI (50 mg, 0.26 mmol) and stirred at room temperature for 10 minutes. This solution was charged with (S)-2-(8-(4-aminobutoxy)-6-(4-chlorophenyl)-1-methyl-4H-benzo[f][1,2,4]triazolo[4,3-a][1,4]diazepin-4-yl)-N-ethylacetaminde (100 mg, 0.21 mmol) and DMAP (32 mg, 0.26 mmol) resulting in 6 mg, 5% yield of the title compound as an off white solid. ¹H NMR (400 MHz, CD₃OD): δ 7.69 (d, J=8.4 Hz, 1H), 7.53 (d, J=8.4 Hz, 2H), 7.44-7.34 (m, 3H), 7.27-7.23 (m, 1H), 7.19-7.14 (m, 1H), 6.92-6.88 (m, 1H), 6.77 (d, J=8.4 Hz, 1H), 4.64 (dd, J=8.8, 5.6 Hz, 1H), 4.12-4.00 (m, 2H), 3.45-3.35 (m, 4H), 3.28-3.20 (m, 2H), 2.65 (s, 3H), 1.90-1.71 (m, 4H), 1.18 (t, J=7.2 Hz, 3H). Mol. Wt: 617.09; MS (ES+): m/z 617.20[MH⁺].

### Synthesis of (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde (BRD-C-27)

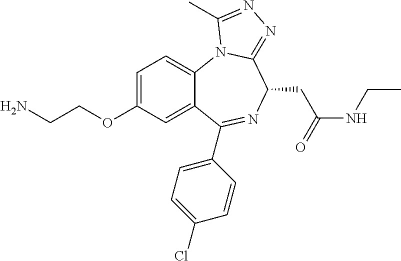 [SMILES: CCNC(=O)C[C@@H]1N=C(c2ccc(Cl)cc2)c2cc(OCCN)ccc2-n2c(C)nnc21]

General Procedure for Boc-Deprotection:

A solution of (S)-tert-butyl (2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate (500 mg, 0.852 mmol) in 1,4-dioxane (10 mL) was charged with conc. HCl (0.5 mL) and stirred for at room temperature for 4-6 h. The reaction mixture was concentrated in vacuo to obtain a residue which was triturated with diethyl ether and filtered through a fritted funnel resulting in 400 mg, 98% yield of the title compound as an off white solid as a hydrochloride salt. ¹H NMR (400 MHz, CD₃OD): δ 7.78 (d, J=8.8 Hz, 1H), 7.54 (d, J=8.8 Hz, 2H), 7.47 (dd, J=8.8, 2.8 Hz, 1H), 7.41 (d, J=8.8 Hz, 2H), 7.04 (d, J=2.8 Hz, 1H), 4.64 (dd, J=8.8, 5.2 Hz, 1H), 4.32-4.20 (m, 2H), 3.45-3.35 (m, 3H), 3.30-3.20 (m, 2H), 2.67 (s, 3H), 1.19 (t, J=7.2 Hz, 3H). Mol. Wt: 452.94; MS (ES+): m/z 453.15[MH⁺].

The following compounds have been synthesized and purified using the general procedure described above for the synthesis of (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde.

### Synthesis of (S)-2-(8-(3-aminopropoxy)-6-(4-chlorophenyl)-1-methyl-4H-benzo[f][1,2,4]triazolo[4,3-a][1,4]diazepin-4-yl)-N-ethylacetaminde (BRD-C-30)

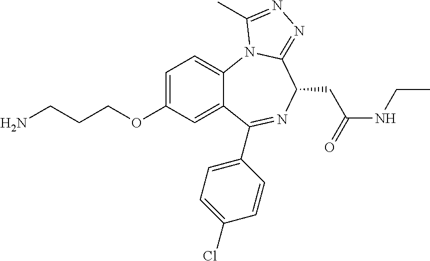 [SMILES: CCNC(=O)C[C@@H]1N=C(c2ccc(Cl)cc2)c2cc(OCCCN)ccc2-n2c(C)nnc21]

Procedure is the same as described for the synthesis of (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde except replacing (S)-tert-butyl(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate with (S)-tert-butyl(3-((6-(4-chlorophenyl)-4-(2-(ethylamino)-2-oxoethyl)-1-methyl-4H-benzo[f][1,2,4]triazolo[4,3-a][1,4]diazepin-8-yl)oxy)propyl)carbamate resulting in 500 mg, 94% yield of the title compound as a yellow solid (hydrochloride salt). ¹H NMR (400 MHz, CD₃OD): δ 7.74 (d, J=8.8 Hz, 1H), 7.54 (d, J=8.8 Hz, 2H), 7.46-7.36 (m, 3H), 6.96 (d, J=2.8, 1H), 4.61 (dd, J=9.2, 5.2 Hz, 1H), 4.20-4.18 (m, 2H), 3.13 (t, J=7.2 Hz, 2H), 2.63 (s, 3H), 2.18-2.08 (m, 2H), 1.19 (t, J=7.2 Hz, 3H). Mol. Wt: 466.96; MS (ES+): m/z 467.20[MH⁺].

### Synthesis of (S)-2-(8-(4-aminobutoxy)-6-(4-chlorophenyl)-1-methyl-4H-benzo[f][1,2,4]triazolo[4,3-a][1,4]diazepin-4-yl)-N-ethylacetaminde (BRD-C-32)

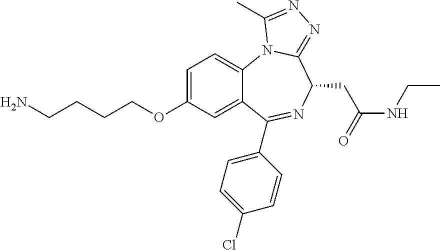 [SMILES: CCNC(=O)C[C@@H]1N=C(c2ccc(Cl)cc2)c2cc(OCCCCN)ccc2-n2c(C)nnc21]

Procedure is the same as described for the synthesis of (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde except replacing (S)-tert-butyl(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate with (S)-tert-butyl(4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butyl)carbamate resulting in 450 mg, 96% yield of the title compound as a white solid (hydrochloride salt). ¹H NMR (400 MHz, CD₃OD): δ 7.72 (d, J=8.8 Hz, 1H), 7.53 (d, J=8.4 Hz, 2H), 7.46-7.34 (m, 3H), 6.19 (d, J=2.8 Hz, 1H), 4.62 (dd, J=9.2, 5.2 Hz, 1H), 4.14-3.98 (m, 2H), 3.02-2.94 (m, 2H), 2.63 (s, 3H), 1.94-1.78 (m, 4H), 1.19 (t, J=7.2 Hz, 3H). Mol. Wt: 480.99; MS (ES+): m/z 481.30[MH⁺].

### Synthesis of (S)-tert-butyl(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate (BRD-C-51)

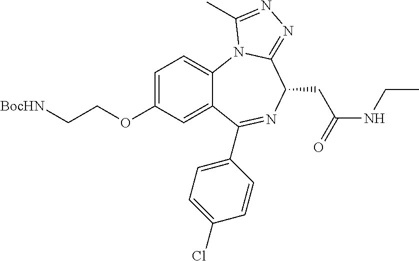 [SMILES: CCNC(=O)C[C@@H]1N=C(c2ccc(Cl)cc2)c2cc(OCCNC(=O)OC(C)(C)C)ccc2-n2c(C)nnc21]

General Procedure for O-Alkylation:

A solution of (S)-2-(6-(4-chlorophenyl)-8-hydroxy-1-methyl-4H-benzo[f][1,2,4]triazolo[4,3-a][1,4]diazepin-4-yl1)-N-ethylacetamide (500 mg, 1.21 mmol) in DMF was charged with potassium carbonate (337 mg, 2.43 mmol) and 2-((tert-butoxycarbonyl)amino)ethyl 4-methylbenzenesulfonate (576 mg, 1.82 mmol) under inert atmosphere and heated at 80° C. for 6-10 h. The reaction mixture was cooled to room temperature and partitioned between water (25 mL) and ethyl acetate and separated. The aqueous was re-extracted with ethyl acetate (2×20 mL) and the combined organic fractions were dried over anhydrous Na₂SO₄, filtered and concentrated in vacuo resulting in a crude product which was purified by column chromatography on silica gel (230-400 mesh), eluting with 5% methanol in chloroform to afford 70, yield: 10% as a white solid. ¹H NMR (400 MHz, CD₃OD): δ 7.71 (d, J=8.8 Hz, 1H), 7.54 (d, J=8.8 Hz, 2H), 7.44-7.35 (m, 3H), 6.96 (s, 1H), 4.63 (dd, J=9.2, 5.2 Hz, 1H), 4.43 (t, J=8.0 Hz, 1H), 4.08-3.98 (m, 2H), 3.59 (t, J=8.0 Hz, 1H), 3.46-3.36 (m, 2H), 3.26-3.18 (m, 2H), 2.64 (s, 3H), 1.42 (s, 9H), 1.19 (t, J=7.2 Hz, 3H). Mol. Wt: 553.05, MS (ES+): m/z 553.35[MH⁺].

The following compounds have been synthesized and purified using the general procedure described above for the synthesis of (S)-tert-butyl(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate.

### Synthesis of (S)-tert-butyl(3-((6-(4-chlorophenyl)-4-(2-(ethylamino)-2-oxoethyl)-1-methyl-4H-benzo[f][1,2,4]triazolo[4,3-a][1,4]diazepin-8-yl)oxy)propyl)carbamate (BRD-C-52)

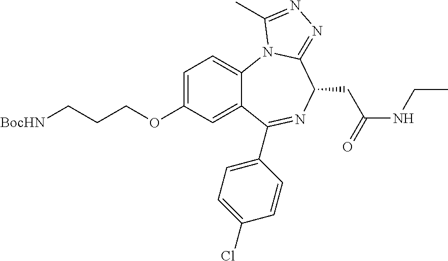 [SMILES: CCNC(=O)C[C@@H]1N=C(c2ccc(Cl)cc2)c2cc(OCCCNC(=O)OC(C)(C)C)ccc2-n2c(C)nnc21]

Procedure was the same as the synthesis of (S)-tert-butyl(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate except using 3-((tert-butoxycarbonyl)amino)propyl benzenesulfonate in place of 2-((tert-butoxycarbonyl)amino)ethyl 4-methylbenzenesulfonate resulting in 130 mg, yield: 94% as an off white solid. ¹H NMR (400 MHz, CDCl₃): δ 7.48 (d, J=8.0 Hz, 2H), 7.40-7.30 (m, 3H), 7.18 (d, J=6.8 Hz, 1H), 6.84 (s, 1H), 6.36 (bs, 1H), 4.61 (t, J=6.4 Hz, 1H), 4.02-3.94 (m, 2H), 3.55-3.44 (m, 1H), 3.41-3.24 (m, 5H), 2.61 (s, 3H), 2.02-1.92 (m, 2H), 1.43 (s, 9H), 1.80 (t, J=7.2 Hz, 3H). Mol. Wt: 567.08; MS (ES+): m/z 567.20[MH⁺].

### Synthesis of (S)-tert-butyl(4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butyl)carbamate (BRD-C-53)

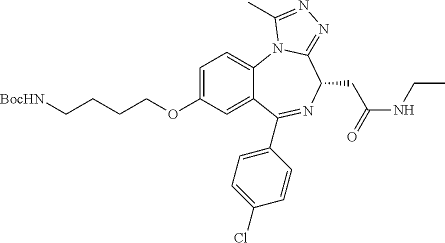 [SMILES: CCNC(=O)C[C@@H]1N=C(c2ccc(Cl)cc2)c2cc(OCCCCNC(=O)OC(C)(C)C)ccc2-n2c(C)nnc21]

Procedure was the same as for the synthesis of (S)-tert-butyl(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate except using 4-((tert-butoxycarbonyl)amino)butyl benzenesulfonate in place of 2-((tert-butoxycarbonyl)amino)ethyl 4-methylbenzenesulfonate resulting in 20 mg, yield: 95% as a white solid. ¹H NMR (400 MHz, CDCl₃): δ 7.48 (d, J=8.4 Hz, 2H), 7.40-7.30 (m, 3H), 7.17 (dd, J=8.4, 2.4 Hz, 1H), 6.83 (d, J=2.4 Hz, 1H), 6.35 (bs, 1H), 4.60 (t, J=7.2 Hz, 2H), 4.00-3.88 (m, 2H), 3.55-3.46 (m, 1H), 3.42-3.24 (m, 3H), 3.22-3.14 (m, 2H), 2.61 (s, 3H), 1.85-1.75 (m, 2H), 1.70-1.61 (m, 2H), 1.44 (s, 9H), 1.18 (t, J=7.2 Hz, 3H). Mol. Wt: 581.11; MS (ES+): m/z 581.20[MH⁺].

### Synthesis of (S)—N-(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)-2,3-dihydroxybenzamide (BRD-N-25)

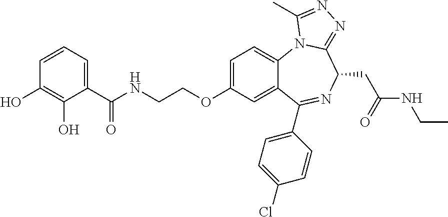 [SMILES: CCNC(=O)C[C@@H]1N=C(c2ccc(Cl)cc2)c2cc(OCCNC(=O)c3cccc(O)c3O)ccc2-n2c(C)nnc21]

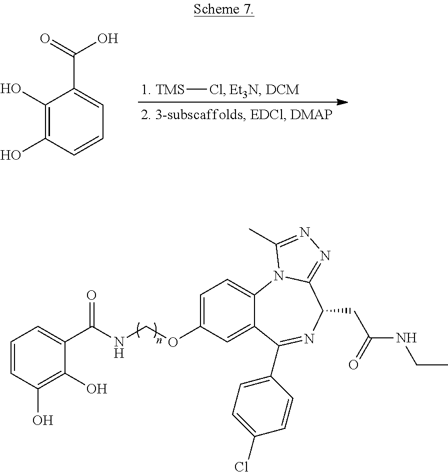

A solution of 2,3-dihydroxy benzoic acid (71 mg, 0.464 mmol) in anhydrous DCM (15 mL) was charged with triethylamine (0.25 mL, 1.85 mmol) and trimethylchlorosilane (0.17 mL, 1.38 mmol) and stirred at room temperature overnight. This solution was charged with EDCI (44 mg, 0.231 mmol) and DMAP (28 mg, 0.231 mmol) and stirred for an additional for 10 minutes then charged with (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde (70 mg, 0.154 mmol) and stirred at room temperature for 10-12 h. The reaction mixture was partitioned between DCM and water and separated. The aqueous was re-extracted with DCM (3×20 mL) and the combined organic fractions were dried over anhydrous Na₂SO₄, filtered and concentrated in vacuo resulting in a crude product which was purified by preparative HPLC to afford 15 mg, 16% yield of the title compound as an off white solid. ¹H NMR (400 MHz, CD₃OD): δ 7.72 (d, J=9.2 Hz, 1H), 7.53 (d, J=8.4 Hz, 2H), 7.45 (dd, J=9.2, 2.8 Hz, 1H), 7.39 (d, J=8.4 Hz, 2H), 7.18 (d, J=8.0 Hz, 1H), 7.02 (d, J=2.8 Hz, 1H), 6.94 (d, J=8.0 Hz, 1H), 6.71 (t, J=8.0 Hz, 1H) 4.64 (dd, J=8.4, 5.4 Hz, 1H), 4.30-4.18 (m, 2H), 3.76 (t, J=5.4 Hz, 2H), 3.43-3.35 (m, 1H), 3.29-3.19 (m, 3H), 2.69 (s, 3H), 1.18 (t, J=7.2 Hz, 3H). Mol. Wt: 589.04, MS (ES+): m/z 589.15[MH⁺].

The following compounds have been synthesized and purified using the general procedure described above for the synthesis of (S)—N-(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)-2,3-dihydroxybenzamide.

### Synthesis of (S)—N-(3-((6-(4-chlorophenyl)-4-(2-(ethylamino)-2-oxoethyl)-1-methyl-4H-benzo[f][1,2,4]triazolo[4,3-a][1,4]diazepin-8-yl)oxy)propyl)-2,3-dihydroxybenzamide (BRD-N-27)

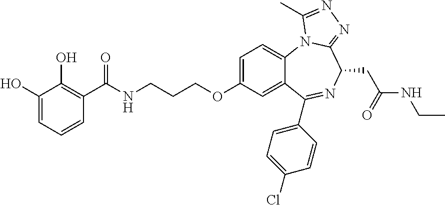 [SMILES: CCNC(=O)C[C@@H]1N=C(c2ccc(Cl)cc2)c2cc(OCCCNC(=O)c3cccc(O)c3O)ccc2-n2c(C)nnc21]

The procedure is the same as that used for the synthesis of (S)—N-(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)-2,3-dihydroxybenzamide except using (S)-2-(8-(3-aminopropoxy)-6-(4-chlorophenyl)-1-methyl-4H-benzo[f][1,2,4]triazolo[4,3-a][1,4]diazepin-4-yl)-N-ethylacetaminde in place of (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde resulting in 15 mg, 17% yield of the title compound as a brown solid. ¹H NMR (400 MHz, CD₃OD): δ 7.70 (d, J=8.8 Hz, 1H), 7.52 (d, J=8.0 Hz, 2H), 7.42-7.30 (m, 3H), 7.16 (d, J=8.0 Hz, 1H), 6.95-6.85 (m, 2H), 6.69 (t, J=8.0 Hz, 1H), 4.68 (dd, J=8.4, 6.2 Hz, 1H), 4.20-4.12 (m, 2H), 3.58 (t, J=6.2 Hz, 2H), 2.70 (s, 3H), 2.12-2.03 (m, 2H), 1.18 (t, J=7.2 Hz, 3H). Mol. Wt: 603.07; MS (ES+): m/z 603.15[MH⁺].

### Synthesis of (S)—N-(4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butyl)-2,3-dihydroxybenzamide (BRD-N-29)

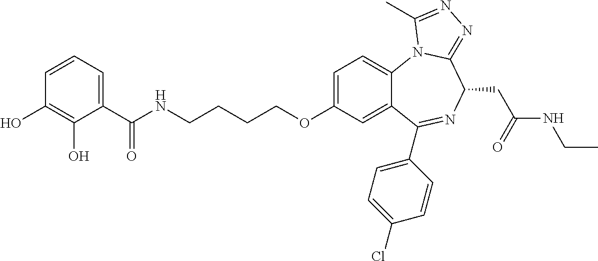 [SMILES: CCNC(=O)C[C@@H]1N=C(c2ccc(Cl)cc2)c2cc(OCCCCNC(=O)c3cccc(O)c3O)ccc2-n2c(C)nnc21]

Procedure is the same as used for the synthesis of (S)—N-(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)-2,3-dihydroxybenzamide except using (S)-2-(8-(4-aminobutoxy)-6-(4-chlorophenyl)-1-methyl-4H-benzo[f][1,2,4]triazolo[4,3-a][1,4]diazepin-4-yl)-N-ethylacetaminde in place of (S)-2-(8-(2-aminoethoxy)-6-(4-chlorophenyl)-1-methyl-4H-benzo[f][1,2,4]triazolo[4,3-a][1,4]diazepin-4-yl)-N-ethylacetaminde resulting in 6 mg, 12% yield of the title compound as an off white solid. ¹H NMR (400 MHz, CD₃OD): δ 7.68 (d, J=8.8 Hz, 1H), 7.53 (d, J=8.4 Hz, 2H), 7.47-7.32 (m, 3H), 7.21-7.08 (m, 2H), 6.95-6.88 (m, 1H), 6.72-6.66 (m, 1H), 4.65 (dd, J=8.8, 5.6 Hz, 1H), 4.14-4.00 (m, 2H), 3.50-3.34 (m, 4H), 2.67 (s, 3H), 1.90-1.72 (m, 4H), 1.18 (t, J=7.2 Hz, 3H). Mol. Wt: 617.09; MS (ES+): m/z 617.25[MH⁺].

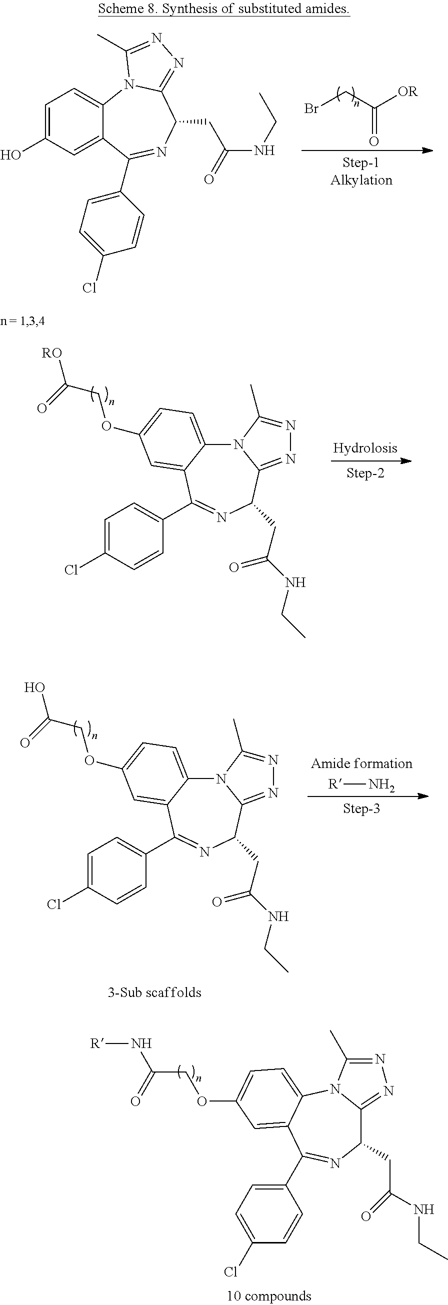

General Procedure for Amide Formation:

A solution of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid (80 mg, 0.17 mmol) in DCM (10 mL) was charged with EDCI (50 mg, 0.25 mmol) and stirred at room temperature for 10 minutes. This solution was charged with aniline (19 mg, 0.205 mmol) and DMAP (31 mg, 0.25 mmol) and stirred at room temperature for 15 h. The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 11 mg, 12% yield of the title compound as off white solid. ¹H NMR (400 MHz, CDCl₃): δ 8.11 (s, 1H), 7.56 (d, J=7.6 Hz, 2H), 7.46 (d, J=8.0 Hz, 2H), 7.42-7.35 (m, 2H), 7.31 (d, J=8.4 Hz, 2H), 7.22-7.16 (m, 1H), 7.00 (s, 1H), 6.36 (s, 1H), 4.70-4.55 (m, 3H), 3.56-3.22 (m, 5H), 2.63 (s, 3H), 1.18 (t, J=7.2 Hz, 3H). Mol. Wt: 543.02; MS (ES+): m/z 543.20[MH⁺].

The following compounds have been synthesized and purified using the general procedure described above for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide.

### Synthesis of (S)-(3-(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetamido)phenyl)boronic acid (BRD-E-15)

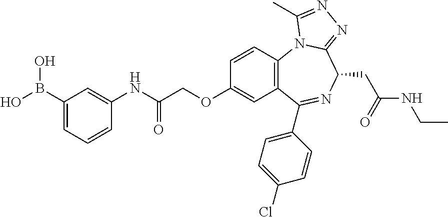 [SMILES: CCNC(=O)C[C@@H]1N=C(c2ccc(Cl)cc2)c2cc(OCC(=O)Nc3cccc(B(O)O)c3)ccc2-n2c(C)nnc21]

Procedure was the same used for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide except using (3-aminophenyl)boronic acid in place of aniline resulting in 80 mg, 80% yield of the title compound as off white solid. ¹H NMR (400 MHz, CD₃OD): δ 7.79 (d, J=8.4 Hz, 2H), 7.61 (d, J=7.6 Hz, 1H), 7.56-7.49 (m, 3H), 7.45-7.32 (m, 2H), 7.26 (d, J=8.4 Hz, 2H), 7.08 (d, J=2.8 Hz, 1H), 4.77 (ABq, J=15.4 Hz, 2H), 4.68 (dd, J=8.8, 5.2 Hz, 1H), 3.45-3.36 (m, 1H), 3.29-3.20 (m, 3H), 2.72 (s, 3H), 1.18 (t, J=7.2 Hz, 3H). Mol. Wt: 586.83; MS (ES+): m/z 587.15[MH⁺].

### Synthesis of (S)-(4-(2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetamido)phenyl)boronic acid (BRD-E-16)

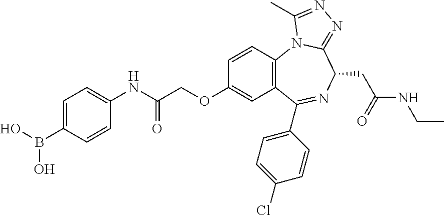 [SMILES: CCNC(=O)C[C@@H]1N=C(c2ccc(Cl)cc2)c2cc(OCC(=O)Nc3ccc(B(O)O)cc3)ccc2-n2c(C)nnc21]

Procedure was the same used for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide using (4-aminophenyl)boronic acid in place of aniline resulting in 5 mg, 14% yield of the title compound as off white solid. ¹H NMR (400 MHz, CD₃OD): δ 7.77 (d, J=9.2 Hz, 2H), 7.63 (d, J=7.6 Hz, 2H), 7.60-7.45 (m, 4H), 7.25 (d, J=8.4 Hz, 2H), 7.05 (s, 1H), 4.82-4.60 (m, 3H), 2.66 (s, 3H), 1.18 (t, J=7.2 Hz, 3H). Mol. Wt: 586.83; MS (ES+): m/z 587.05[MH⁺].

### Synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(2,3-dihydroxyphenyl)acetamide (BRD-N-17)

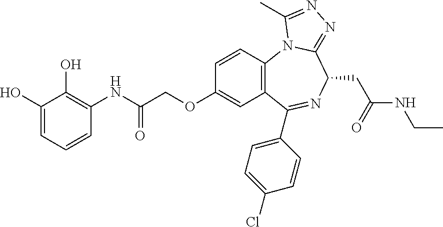 [SMILES: CCNC(=O)C[C@@H]1N=C(c2ccc(Cl)cc2)c2cc(OCC(=O)Nc3cccc(O)c3O)ccc2-n2c(C)nnc21]

Procedure was the same used for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide using 3-aminobenzene-1,2-diol in place of aniline resulting in 13 mg, 15% yield of the title compound as brown solid. ¹H NMR (400 MHz, CD₃OD): δ 7.79 (d, J=9.2 Hz, 1H), 7.56-7.49 (m, 3H), 7.31 (d, J=6.8 Hz, 1H), 7.27 (d, J=8.4 Hz, 2H), 7.08 (d, J=2.4 Hz, 1H), 6.72-6.62 (m, 2H), 4.80-4.72 (m, 2H), 4.67 (dd, J=8.4, 5.6 Hz, 1H), 3.45-3.35 (m, 1H), 3.28-3.20 (m, 3H), 2.69 (s, 3H), 1.18 (t, J=7.2 Hz, 3H). Mol. Wt: 575.01; MS (ES+): m/z 575.15[MH⁺].

### Synthesis of (S)-2-((6-(4-chlorophenyl)-4-(2-(ethylamino)-2-oxoethyl)-1-methyl-4H—(S)-(3-(4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanamido)phenyl)boronic acid (BRD-E-19)

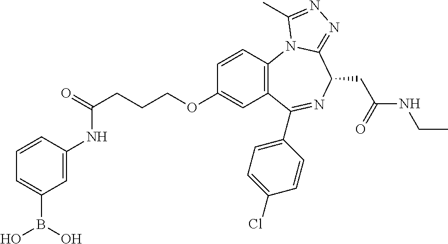 [SMILES: CCNC(=O)C[C@@H]1N=C(c2ccc(Cl)cc2)c2cc(OCCCC(=O)Nc3cccc(B(O)O)c3)ccc2-n2c(C)nnc21]

Procedure was the same as for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide except using (S)-4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanoic acid in place of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid and using (3-aminophenyl)boronic acid in place of aniline using resulting in 60 mg, 96% yield of the title compound as white solid. ¹H NMR (400 MHz, CD₃OD): δ 7.77-7.70 (m, 2H), 7.60-7.50 (m, 3H), 7.42-7.26 (m, 5H), 6.93 (d, J=2.4 Hz, 1H), 4.67 (dd, J=8.4, 5.6 Hz, 1H), 4.20-4.06 (m, 2H), 3.44-3.34 (m, 1H), 3.30-3.20 (m, 3H), 2.71 (s, 3H), 2.55 (t, J=6.8 Hz, 2H), 2.21-2.11 (m, 2H), 1.18 (t, J=7.2 Hz, 3H). Mol. Wt: 614.89; MS (ES+): m/z 615.20[MH¹].

### Synthesis of (S)-4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(2,3-dihydroxyphenyl)butanamide (BRD-N-21)

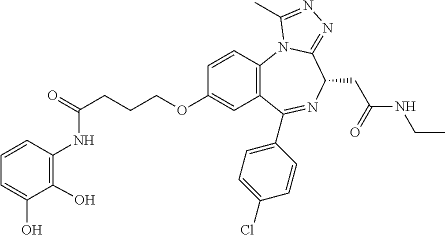 [SMILES: CCNC(=O)C[C@@H]1N=C(c2ccc(Cl)cc2)c2cc(OCCCC(=O)Nc3cccc(O)c3O)ccc2-n2c(C)nnc21]

Procedure was the same as for the synthesis of (S)-(3-(4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanamido)phenyl)boronic acid except using 3-aminobenzene-1,2-diol in place of (3-aminophenyl)boronic acid resulting in 13 mg, 15% yield of the title compound as brown solid. ¹H NMR (400 MHz, CD₃OD): δ 7.70 (d, J=8.8 Hz, 1H), 7.51 (d, J=8.4 Hz, 2H), 7.42-7.32 (m, 3H), 6.96-6.82 (m, 2H), 6.62 (d, J=3.6 Hz, 2H), 4.66 (dd, J=8.0, 5.6 Hz, 1H), 4.20-4.06 (m, 2H), 3.42-3.34 (m, 1H), 3.28-3.20 (m, 3H), 2.68 (s, 3H), 2.65-2.58 (m, 2H), 2.22-2.12 (m, 2H), 1.18 (t, J=6.8 Hz, 3H). Mol. Wt: 603.07; MS (ES+): m/z 603.20[MH⁺].

### Synthesis of (S)-(3-(5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanamido)phenyl)boronic acid (BRD-E-20)

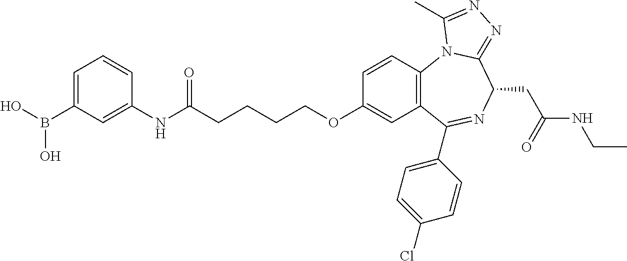 [SMILES: CCNC(=O)C[C@@H]1N=C(c2ccc(Cl)cc2)c2cc(OCCCCC(=O)Nc3cccc(B(O)O)c3)ccc2-n2c(C)nnc21]

Procedure is the same as for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide except using (S)-5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid in place of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid and using (3-aminophenyl)boronic acid in place of aniline resulting in 15 mg, 15% yield of the title compound as white solid. ¹H NMR (400 MHz, CD₃OD): δ 7.75 (s, 1H), 7.68 (d, J=9.2 Hz, 1H), 7.62-7.50 (m, 3H), 7.44-7.26 (m, 5H), 6.91 (s, 1H), 4.66-4.56 (m, 1H), 4.12-3.90 (m, 2H), 3.44-3.36 (m, 1H), 3.26-3.16 (m, 3H), 2.62 (s, 3H), 2.50-2.36 (m, 2H), 1.94-1.78 (m, 4H), 1.18 (t, J=7.2 Hz, 3H). Mol. Wt: 628.91; MS (ES+): m/z 629.25[MH⁺].

### Synthesis of (S)-5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(2,3-dihydroxyphenyl)pentanamide (BRD-N-23)

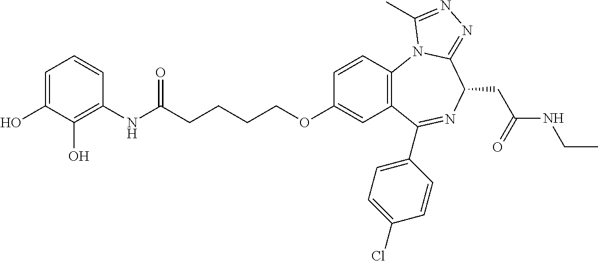 [SMILES: CCNC(=O)C[C@@H]1N=C(c2ccc(Cl)cc2)c2cc(OCCCCC(=O)Nc3cccc(O)c3O)ccc2-n2c(C)nnc21]

Procedure is the same as for the synthesis of (S)-2-(6-(4-chlorophenyl)-1-methyl-8-(2-oxo-2-(phenylamino)ethoxy)-4H-benzo[f][1,2,4]triazolo[4,3-a][1,4]diazepin-4-yl)-N-ethylacetamide except using (S)-5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid in place of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid and using 3-aminobenzene-1,2-diol in place of aniline resulting in 15 mg, 25% yield of the title compound as off white solid. ¹H NMR (400 MHz, CD₃OD): δ 7.71 (d, J=9.2 Hz, 1H), 7.54 (d, J=8.0 Hz, 2H), 7.44-7.34 (m, 3H), 6.94 (d, J=2.4 Hz, 1H), 6.92-6.84 (m, 1H), 6.68-6.58 (m, 2H), 4.68 (dd, J=6.0, 2.8 Hz, 1H), 4.14-4.02 (m, 2H), 3.44-3.34 (m, 1H), 3.29-3.22 (m, 3H), 2.71 (s, 3H), 2.56-2.48 (m, 2H), 1.91-1.85 (m, 4H), 1.18 (t, J=7.2 Hz, 3H). Mol. Wt: 617.09; MS (ES+): m/z 617.20[MH⁺].

### Synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid (BRD-C-44)

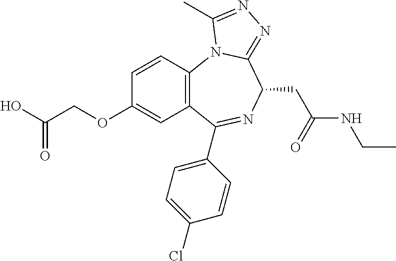 [SMILES: CCNC(=O)C[C@@H]1N=C(c2ccc(Cl)cc2)c2cc(OCC(=O)O)ccc2-n2c(C)nnc21]

General Procedure for Hydrolysis:

A solution of (S)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate (200 mg, 0.403 mmol) in THF/H₂O (20 mL) was charged with LiOH (67 mg, 1.61 mmol) and stirred at room temperature for 4 to 6 h. The solvent was removed under reduced pressure to afford a residue which was acidified with 10% citric acid solution (10 mL) and extracted with ethyl acetate (3×10 mL) and the combined organic fractions were dried over anhydrous Na₂SO₄, filtered and concentrated in vacuo resulting in a crude product which was purified by preparative HPLC to afford 150 mg, 80% yield of the title compound as white solid. ¹H NMR (400 MHz, DMSO-d₆): δ 13.00 (bs, 1H), 8.24-8.14 (m, 1H), 7.78 (d, J=8.8 Hz, 1H), 7.49 (AB q, J=9.0 Hz, 4H), 7.36 (dd, J=8.8, 2.4 Hz, 1H), 6.87 (d, J=2.4 Hz, 1H), 4.77 (s, 2H), 4.51-4.43 (m, 1H), 3.29-3.05 (m, 4H), 2.53 (s, 3H), 1.06 (t, J=7.2 Hz, 3H). Mol. Wt: 467.90; MS (ES+): m/z 468.15[MH⁺].

The following compounds have been synthesized and purified using the general procedure described above for the synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid.

### Synthesis of (S)-4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanoic acid (BRD-C-48)

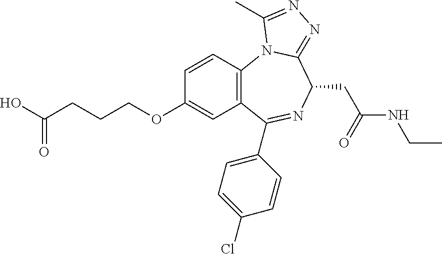 [SMILES: CCNC(=O)C[C@@H]1N=C(c2ccc(Cl)cc2)c2cc(OCCCC(=O)O)ccc2-n2c(C)nnc21]

Procedure was the same as for the synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid except using (S)-ethyl 4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanoate in place of (5)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate resulting in 80 mg, 84% yield of the title compound as white solid. ¹H NMR (400 MHz, CD₃OD): δ 7.71 (d, J=8.8 Hz, 1H), 7.54 (d, J=8.4 Hz, 2H), 7.45-7.35 (m, 3H), 6.92 (d, J=2.4 Hz, 1H), 4.63 (dd, J=8.8, 5.2 Hz, 1H), 4.12-4.00 (m, 2H), 2.63 (s, 3H), 2.46 (t, J=6.8 Hz, 2H), 2.12-2.00 (m, 2H), 1.18 (t, J=7.2 Hz, 3H). Mol. Wt: 495.96; MS (ES+): m/z 496.25[MH⁺].

### Synthesis of (S)-5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid (BRD-C-50)

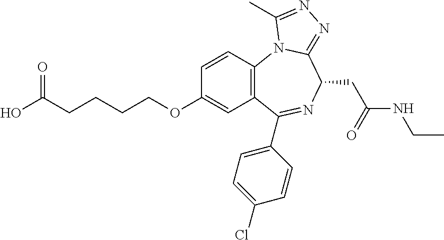 [SMILES: CCNC(=O)C[C@@H]1N=C(c2ccc(Cl)cc2)c2cc(OCCCCC(=O)O)ccc2-n2c(C)nnc21]

Procedure was the same as used in the synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid except using (S)-ethyl 5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoate in place of (S)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate resulting in 120 mg, 70% yield of the title compound as white solid. ¹H NMR (400 MHz, CD₃OD): δ 7.70 (d, J=8.8 Hz, 1H), 7.54 (d, J=8.4 Hz, 2H), 7.41 (d, J=8.4 Hz, 2H), 7.37 (dd, J=8.8, 2.4 Hz, 1H), 6.91 (d, J=2.4 Hz, 1H), 4.62 (dd, J=8.8, 4.8 Hz, 1H), 4.10-3.96 (m, 2H), 3.49-3.34 (m, 2H), 3.28-3.18 (m, 2H), 2.63 (s, 3H), 2.35 (t, J=6.8 Hz, 2H), 1.87-1.70 (m, 4H), 1.18 (t, J=7.2 Hz, 3H). Mol. Wt: 509.98; MS (ES+): m/z 510.40[MH⁺].

### Synthesis of (S)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate (BRD-C-43)

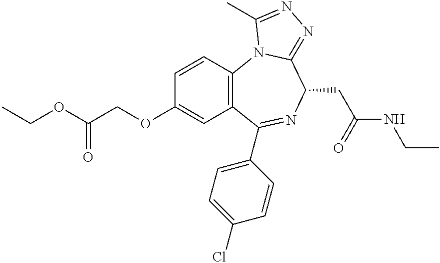 [SMILES: CCNC(=O)C[C@@H]1N=C(c2ccc(Cl)cc2)c2cc(OCC(=O)OCC)ccc2-n2c(C)nnc21]

General Procedure for Ester Formation:

A solution of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid (300 mg, 0.731 mmol), in DMF/acetone/acetonitrile) was charged with base (K₂CO₃/Cs₂CO₃) (303 mg, 2.19 mmol) and ethyl 2-bromoacetate (244 mg, 1.46 mmol) and 18-crown-6 (9 mg, 0.036 mmol) under nitrogen atmosphere and stirred and heated at 50-90° C. for 6-15 h. The reaction mixture was cooled to room temperature, diluted with water (25 mL), extracted with ethyl acetate (3×10 mL) and the combined organic fractions were dried over anhydrous Na₂SO₄, filtered and concentrated in vacuo resulting in a crude product which was purified by silica gel column chromatography eluting with 5% methanol in chloroform to afford 200 mg, 55% yield of the title compound as off white solid. ¹H NMR (400 MHz, CDCl₃): δ 7.46 (d, J=8.4 Hz, 2H), 7.39 (d, J=9.2 Hz, 1H), 7.33 (d, J=8.4 Hz, 2H), 7.25-7.18 (m, 1H), 6.81 (d, J=2.0 Hz, 1H), 6.37 (bs, 1H), 4.68-4.53 (m, 3H), 4.22 (q, J=7.0 Hz, 2H), 3.55-3.46 (m, 1H), 3.41-3.22 (m, 3H), 2.61 (s, 3H), 1.26 (t, J=7.0 Hz, 3H), 1.18 (t, J=7.2 Hz, 3H). Mol. Wt: 495.96; MS (ES+): m/z 496.25 [MH⁺].

The following compounds have been synthesized and purified using the general procedure described above for the synthesis of (S)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate.

### Synthesis of (S)-ethyl 4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanoate (BRD-C-47)

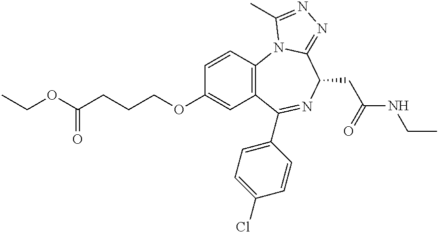 [SMILES: CCNC(=O)C[C@@H]1N=C(c2ccc(Cl)cc2)c2cc(OCCCC(=O)OCC)ccc2-n2c(C)nnc21]

Procedure is the same as for the synthesis of (S)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate except using ethyl 4-bromobutanoate in place of ethyl 2-bromoacetate resulting in 200 mg, 87% yield of the title compound as white solid. ¹H NMR (400 MHz, CDCl₃): δ 7.48 (d, J=8.0 Hz, 2H), 7.40-7.30 (m, 3H), 7.18 (d, J=8.8 Hz, 1H), 6.85 (s, 1H), 6.41 (bs, 1H), 4.61 (t, J=6.8 Hz, 1H), 4.13 (q, J=7.2 Hz, 2H), 4.06-m 3.92 (m, 2H), 3.56-3.46 (m, 1H), 3.41-3.20 (m, 3H), 2.60 (s, 3H), 2.49 (t, J=6.8 Hz, 2H), 2.16-2.04 (m, 2H), 1.18 (t, J=6.8 Hz, 3H). Mol. Wt: 524.01; MS (ES+): m/z 524.15[MH⁺].

### Synthesis of (S)-methyl 5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoate (BRD-C-49)

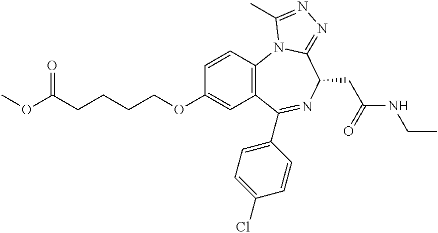 [SMILES: CCNC(=O)C[C@@H]1N=C(c2ccc(Cl)cc2)c2cc(OCCCCC(=O)OC)ccc2-n2c(C)nnc21]

Procedure is the same as used for the synthesis of (5)-ethyl 2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetate except for using methyl 5-bromopentanoate in place of ethyl 2-bromoacetate resulting in 320 mg, 83% yield of the title compound as white solid. ¹H NMR (400 MHz, CDCl₃): δ 7.48 (d, J=8.0 Hz, 2H), 7.40-7.30 (m, 3H), 7.17 (d, J=7.2 Hz, 1H), 6.83 (s, 1H), 6.38 (bs, 1H), 4.61 (t, J=7.2 Hz, 1H), 3.98-3.86 (m, 2H), 3.68 (s, 3H), 3.55-3.46 (m, 1H), 3.42-3.19 (m, 3H), 2.61 (s, 3H), 2.44-2.34 (m, 2H), 1.88-1.74 (m, 4H), 1.18 (t, J=6.8 Hz, 3H). Mol. Wt: 524.01; MS (ES+): m/z 524.20[MH⁺].

Synthesis of Monomers:

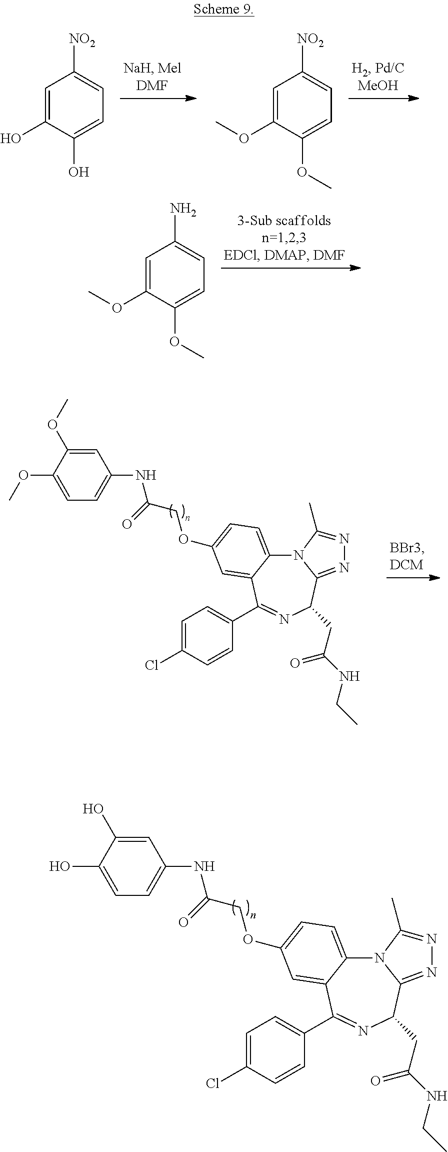

### Synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)acetamide (BRD-N-16)

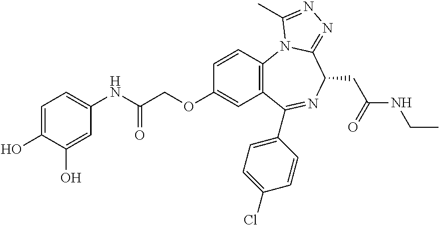 [SMILES: CCNC(=O)C[C@@H]1N=C(c2ccc(Cl)cc2)c2cc(OCC(=O)Nc3ccc(O)c(O)c3)ccc2-n2c(C)nnc21]

General Procedure:

An ice-cooled solution of 3,4-dihydroxy nitrobenzene (100 mg, 0.63 mmol) in anhydrous DMF (15 mL) was charged with sodium hydride (60% dispersion in oil, 101 mg, 2.5 mmol) and methyl iodide (269 mg, 1.89 mmol) and stirred at room temperature for 2 h. The reaction mixture was partitioned between ethyl acetate and water and separated. The aqueous layer was further extracted with ethyl acetate and the combined organic layer was dried over anhydrous sodium sulfate, filtered, and concentrated in vacuo to afford a crude product which was purified by silica gel column chromatography eluting with 5% methanol in chloroform resulting in 78 mg, 68% yield of 3,4-dimethoxy nitrobenzene.

A solution of 3,4-dimethoxy nitrobenzene (100 mg, 0.54 mmol) in methanol (15 mL) was charged with 10% Pd/C (25 mg) under inert atmosphere then charged with H₂ gas at atmospheric pressure (balloon pressure) for 3 h. The reaction mixture was filtered through a pad of celite and the pad was washed with methanol and the filtrate was concentrated in vacuo to afford 3,4-dimethoxy aniline (81 mg, Yield: 97%).

The procedure for amide formation was followed as written in step-3 of the General Procedure for Amide Formation (above) to afford corresponding dimethoxy amide derivative (Yield: 65-85%).

A solution of dimethoxy amide derivative (120 mg, 0.199 mmol) in dichloromethane (15 mL/g) was charged with BBr₃ (149 mg, 0.597 mmol) at 0° C. and allowed to warm to room temperature and stirred for 1 h. The reaction mixture was partitioned between dichloromethane and water and separated the aqueous layer was further extracted with dichloromethane and the combined organic layer was dried over anhydrous sodium sulfate and concentrated in vacuo to afford a crude product which was purified by preparative HPLC to afford 10 mg, 9% yield of the title compound as a white solid. ¹H NMR (400 MHz, CD₃OD): δ 7.77 (d, J=9.2 Hz, 1H), 7.55-7.46 (m, 3H), 7.29 (d, J=8.4 Hz, 2H), 7.11 (s, 1H), 7.05 (s, 1H), 6.80-6.68 (m, 2H), 5.28-5.10 (m, 3H), 3.45-3.35 (m, 1H), 2.67 (s, 3H), 1.18 (t, J=7.2 Hz, 3H). Mol. Wt: 575.01; MS (ES+): m/z 575.05[MH⁺].

The following compounds have been synthesized and purified using the general procedure described above for the synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)acetamide.

### Synthesis of (S)-4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)butanamide (BRD-N-20)

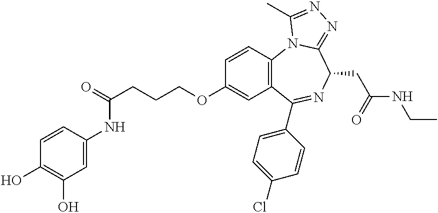 [SMILES: CCNC(=O)C[C@@H]1N=C(c2ccc(Cl)cc2)c2cc(OCCCC(=O)Nc3ccc(O)c(O)c3)ccc2-n2c(C)nnc21]

Procedure is the same as used for the synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)acetamide except using (S)-4-((6-(4-chlorophenyl)-4-(2-(ethylamino)-2-oxoethyl)-1-methyl-4H-benzo[f][1,2,4]triazolo[4,3-a][1,4]diazepin-8-yl)oxy)butanoic acid in place of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid resulting in 16 mg, 13% yield of the title compound as white solid. ¹H NMR (400 MHz, CD₃OD): δ 7.71 (d, J=8.8 Hz, 1H), 7.51 (d, J=7.6 Hz, 2H), 7.45-7.34 (m, 3H), 7.05 (s, 1H), 6.90 (s, 1H), 6.71 (d, J=8.0 Hz, 1H), 6.66 (d, J=8.8 Hz, 1H), 4.74-4.60 (m, 1H), 4.18-4.02 (m, 2H), 3.45-3.36 (m, 1H), 2.71 (s, 3H), 2.54-2.42 (m, 2H), 2.20-2.08 (m, 2H), 1.18 (t, J=7.2 Hz, 3H). Mol. Wt: 603.07; MS (ES+): m/z 603.10[MH⁺].

### Synthesis of (S)-5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)pentanamide (BRD-N-22)

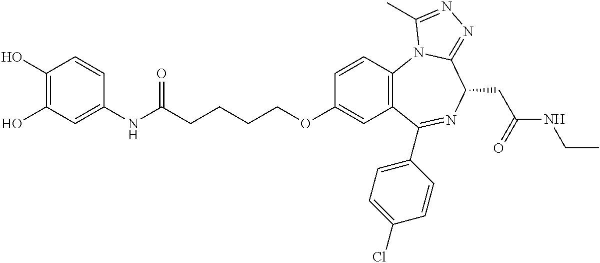 [SMILES: CCNC(=O)C[C@@H]1N=C(c2ccc(Cl)cc2)c2cc(OCCCCC(=O)Nc3ccc(O)c(O)c3)ccc2-n2c(C)nnc21]

Procedure is the same as used for the synthesis of (S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)acetamide except for using (S)-5-((6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid in place of S)-2-((6-(4-chlorophenyl)-4-(2-(ethylamino)-2-oxoethyl)-1-methyl-4H-benzo[f][1,2,4]triazolo[4,3-a][1,4]diazepin-8-yl)oxy)acetic acid resulting in 10 mg, 11% yield of the title compound as brown solid. ¹H NMR (400 MHz, CD₃OD): δ 7.69 (d, J=8.8 Hz, 1H), 7.52 (d, J=8.0 Hz, 2H), 7.43-7.34 (m, 3H), 7.02 (s, 1H), 6.91 (s, 1H), 6.73 (d, J=8.4 Hz, 1H), 6.66 (d, J=8.0 Hz, 1H), 4.64 (dd, J=8.4, 5.6 Hz, 1H), 4.10-4.00 (m, 2H), 3.44-3.34 (m, 1H), 3.29-3.20 (m, 3H) 2.66 (s, 3H), 2.42-2.32 (m, 2H), 1.90-1.78 (m, 4H), 1.18 (t, J=7.2 Hz, 3H). Mol. Wt: 617.09; MS (ES+): m/z 617.25[MH⁺].

### Example 358

This example describes the preparation of monomers.

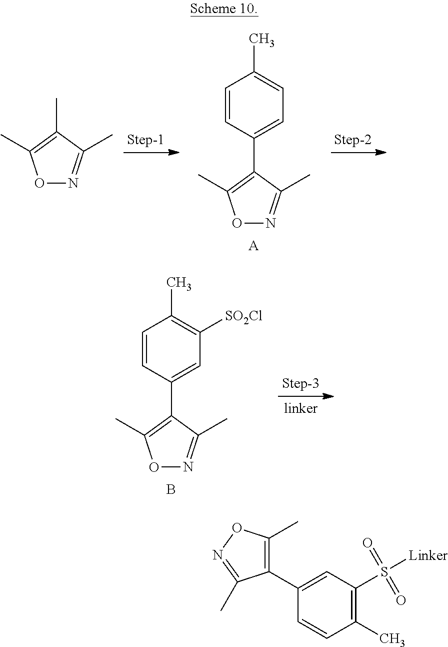

### Synthesis of (3-(5-(3,5-dimethylisoxazol-4-yl)-2-methylphenylsulfonamido)phenyl) boronic acid (BRD-E-57)

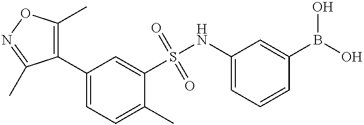 [SMILES: Cc1ccc(-c2c(C)noc2C)cc1S(=O)(=O)Nc1cccc(B(O)O)c1]

A solution of 5-(3,5-dimethylisoxazol-4-yl)-2-methylbenzene-1-sulfonyl chloride (200 mg, 0.70 mmol) in pyridine (10 mL) was charged with (3-aminophenyl)boronic acid (96.1 mg, 0.70 mmol) and stirred at rt for 2 h. The solvent was concentrated under reduced pressure and the reaction mixture was diluted with water (50 mL) and the aqueous was extracted with ethyl acetate (2 x 50 mL). The combined organic extracts were washed with dil HCl solution (10 mL), dried over Na₂SO₄, filtered and concentrated in vacuo resulting in a crude product which was purified by preparative HPLC resulting in 30 mg, 11.11% yield of the title compound as an orange solid. ¹H NMR (400 MHz, DMSO-d₆) δ 7.99 (s, 2H), 7.69 (d, J=1.8 Hz, 1H), 7.53-7.38 (m, 4H), 7.25-7.11 (m, 2H), 2.63 (s, 3H), 2.26 (s, 3H), 2.08 (s, 3H). Mol. Wt: 386.23; MS (ES+): m/z 386.80 [MH⁺], HPLC purity: 95.41% (220 nm).

### Synthesis of (S)-(3-((2-(2-(2-(2-(6-(4-chlorophenyl)-8-methoxy-1-methyl-4H-benzo[f][1,2,4]triazolo[4,3-a][1,4]diazepin-4-yl)acetamido)ethoxy)ethoxy)ethyl)-carbamoyl)-phenyl)-boronic acid (BRD-E-46)

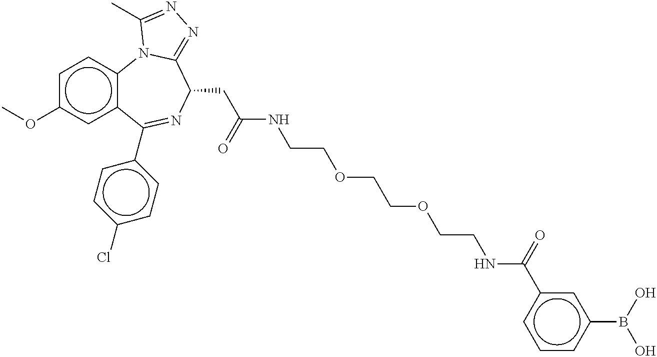

A solution of 3-boronobenzoic acid (23.6 mg, 0.14 mmol) in DCM (5 mL) and DMF (5 mL) were charged with EDCI (40 mg, 0.21 mmol), DMAP (34.7 mg, 0.28 mmol) and stirred at rt for 10 minutes. To this solution, (S)—N-(2-(2-(2-aminoethoxy)ethoxy)ethyl)-2-(6-(4-chlorophenyl)-8-methoxy-1-methyl-4H-benzo[f][1,2,4]triazolo[4,3-a][1,4]diazepin-4-yl)acetamide (76 mg, 0.14 mmol) was added. Weight: 30 mg of the title compound, yield 31.5% as an off white solid. ¹H NMR (400 MHz, DMSO-d₆) δ 7.95-7.73 (m, 3H), 7.62-7.59 (m, 1H), 7.56-7.34 (m, 5H), 6.87 (d, J=2.8 Hz, 2H), 4.50 (dd, J=8.3, 5.7 Hz, 1H), 3.79 (s, 3H), 3.58-3.33 (m, 10H), 3.35-3.09 (m, 4H), 2.55 (s, 3H). Mol. Wt: 674.94; LCMS (m/z): 696.90 [M+Na]. HPLC purity: 87.45% (Max plot).

### Synthesis of (S)—N-(2-(2-(2-(2-(6-(4-chlorophenyl)-8-methoxy-1-methyl-4H-benzo[f][1,2,4]triazolo[4,3-a][1,4]diazepin-4-yl)acetamido)ethoxy)ethoxy)ethyl)-2,3-dihydroxybenzamide (BRD-N-43)

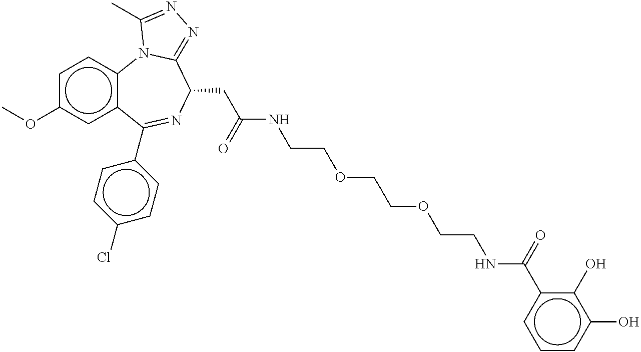

A solution of 2,3-dihydroxybenzoic acid (21.9 mg, 0.14 mmol) in DCM (5 mL) and DMF (5 mL) were charged with EDCI (40 mg, 0.20 mmol), DMAP (34.7 mg, 0.28 mmol) and stirred at rt for 10 minutes. To this solution, (S)—N-(2-(2-(2-aminoethoxy)ethoxy)ethyl)-2-(6-(4-chlorophenyl)-8-methoxy-1-methyl-4H-benzo[f][1,2,4]triazolo[4,3-a][1,4]diazepin-4-yl)acetamide (76 mg, 0.14 mmol) was added. Weight: 7 mg of the title compound, yield 7.4% as an off white solid. ¹H NMR (400 MHz, DMSO-d₆) δ 7.82-7.76 (m, 1H), 7.53-7.45 (m, 6H), 7.38 (dd, J=8.9, 2.9 Hz, 1H), 7.32-7.18 (m, 1H), 6.94-6.84 (m, 2H), 4.54-4.45 (m, 1H), 3.78 (s, 3H), 3.60-3.39 (m, 9H), 3.36-3.09 (m, 5H), 2.55 (s, 3H). Mol. Wt: 663.12; LCMS (m/z): 662.95 [M+]. HPLC purity: 93.25% (Max plot).

### Example 359

This example describes the preparation of monomers.

### Synthesis of (S)-(3-((2-(2-(2-(2-(6-(4-chlorophenyl)-8-methoxy-1-methyl-4H-benzo[f][1,2,4]triazolo[4,3-a][1,4]diazepin-4-yl)acetamido)ethoxy)-ethoxy)ethyl)-carbamoyl)phenyl)-boronic acid (BRD-E-46)

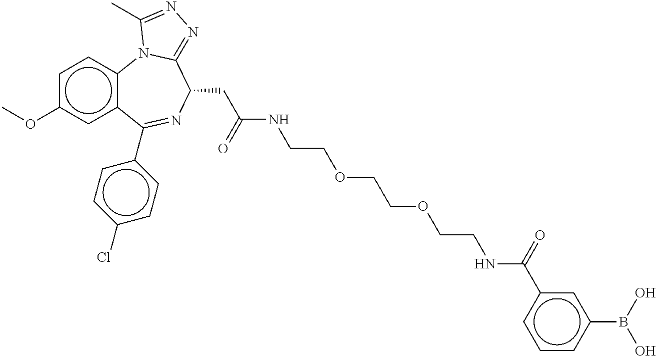

A solution of 3-boronobenzoic acid (23.6 mg, 0.14 mmol) in DCM (5 mL) and DMF (5 mL) was charged with EDCI (40 mg, 0.21 mmol), DMAP (34.7 mg, 0.28 mmol) and stirred at rt for 10 minutes. This solution was charged with (S)—N-(2-(2-(2-aminoethoxy)ethoxy)ethyl)-2-(6-(4-chlorophenyl)-8-methoxy-1-methyl-4H-benzo[f][1,2,4]triazolo[4,3-a][1,4]diazepin-4-yl)acetamide (76 mg, 0.14 mmol). The reaction mixture was partitioned between DCM and H₂O and separated. The aqueous layer was re-extracted with DCM (3×10 mL) and the combined organic fractions were dried over anhydrous Na₂SO₄, filtered, and concentrated in vacuo resulting in a crude product which was purified by preparative HPLC to afford 30 mg, 31.5% yield of the title compound as an off white solid. ¹H NMR (400 MHz, DMSO-d₆) δ 7.95-7.73 (m, 3H), 7.62-7.59 (m, 1H), 7.56-7.34 (m, 5H), 6.87 (d, J=2.8 Hz, 2H), 4.50 (dd, J=8.3, 5.7 Hz, 1H), 3.79 (s, 3H), 3.58-3.33 (m, 10H), 3.35-3.09 (m, 4H), 2.55 (s, 3H). Mol. Wt: 674.94; MS (ES+): m/z: 696.90 [M+Na], HPLC purity: 87.45% (Max plot).

### Synthesis of (S)—N-(2-(2-(2-(2-(6-(4-chlorophenyl)-8-methoxy-1-methyl-4H-benzo[f][1,2,4]triazolo[4,3-a][1,4]diazepin-4-yl)acetamido)ethoxy)ethoxy)ethyl)-2,3-dihydroxybenzamide (BRD-N-43)

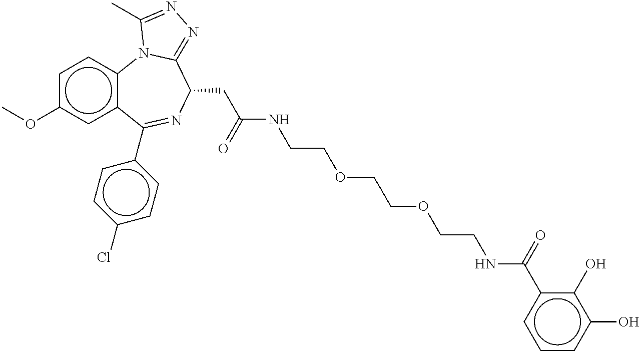

A solution of 2,3-dihydroxybenzoic acid (21.9 mg, 0.14 mmol) in DCM (5 mL) and DMF (5 mL) were charged with EDCI (40 mg, 0.20 mmol), DMAP (34.7 mg, 0.28 mmol) and stirred at rt for 10 minutes. This solution was charged with (S)—N-(2-(2-(2-aminoethoxy)ethoxy)ethyl)-2-(6-(4-chlorophenyl)-8-methoxy-1-methyl-4H-benzo[f][1,2,4]triazolo[4,3-a][1,4]diazepin-4-yl)-acetamide (76 mg, 0.14 mmol). The reaction mixture was partitioned between DCM and H₂O and separated. The aqueous layer was re-extracted with DCM (3×10 mL) and the combined organic fractions were dried over anhydrous Na₂SO₄, filtered and concentrated in vacuo resulting in a crude product which was purified by preparative HPLC to afford 7 mg, 7.4% yield of the title compound as an off white solid. ¹H NMR (400 MHz, DMSO-d₆) δ 7.82-7.76 (m, 1H), 7.53-7.45 (m, 6H), 7.38 (dd, J=8.9, 2.9 Hz, 1H), 7.32-7.18 (m, 1H), 6.94-6.84 (m, 2H), 4.54-4.45 (m, 1H), 3.78 (s, 3H), 3.60-3.39 (m, 9H), 3.36-3.09 (m, 5H), 2.55 (s, 3H). Mol. Wt: 663.12; MS (ES+): m/z: 662.95 [MH⁺], HPLC purity: 93.25% (Max plot).

### Example 360

This example describes the preparation of N-ethyl-2-((4S)-6-(4-mercaptophenyl)-8-methoxy-1-methyl-4H-benzo[f][1,2,4]triazolo[4,3-a][1,4]diazepin-4-yl)acetamide (thio-IBET).

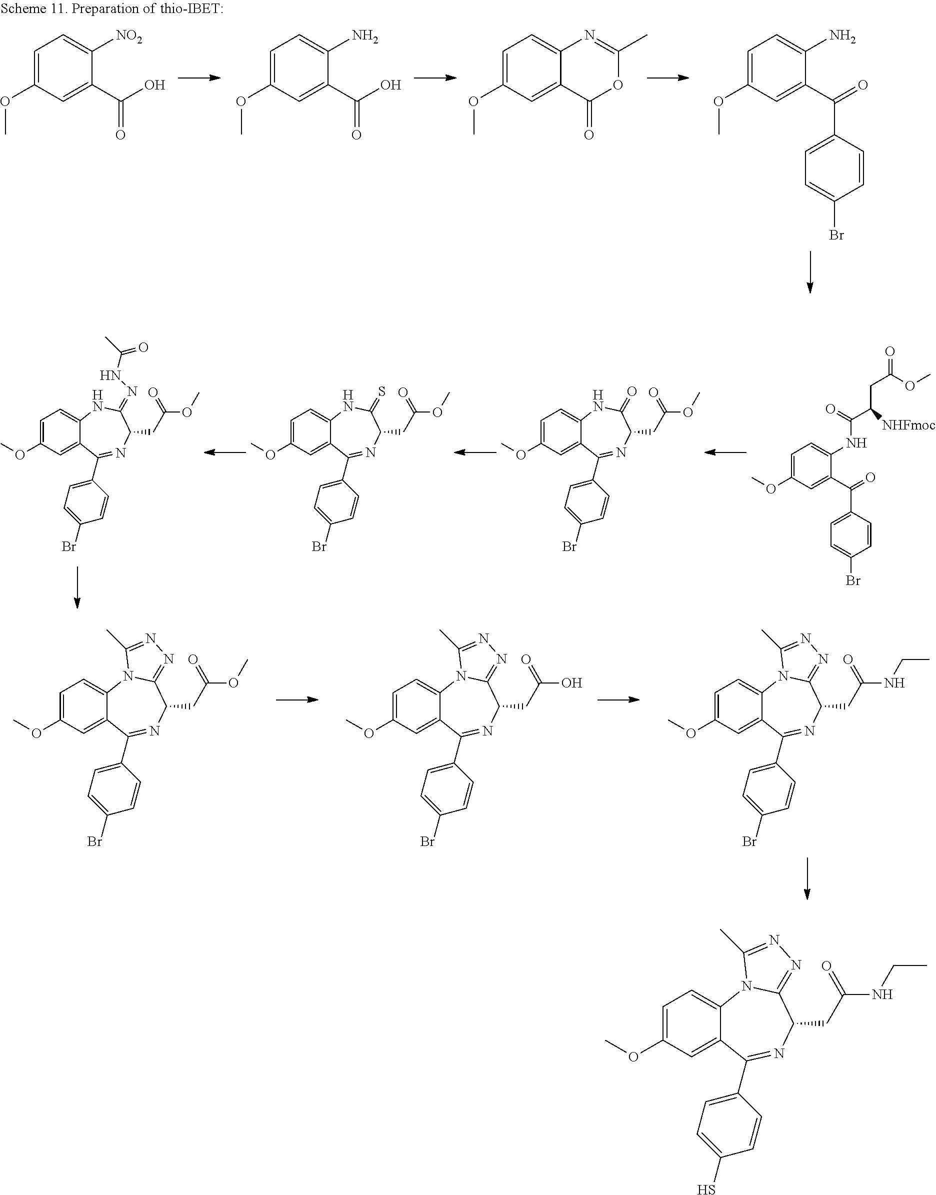

### Synthesis of N-ethyl-2-((4S)-6-(4-mercaptophenyl)-8-methoxy-1-methyl-4H-benzo[f][1,2,4]triazolo[4,3-a][1,4]diazepin-4-yl)acetamide (thio-IBET)

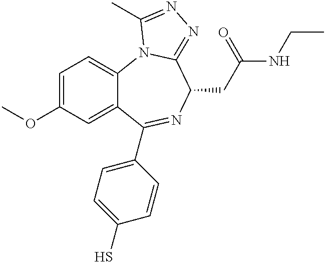 [SMILES: CCNC(=O)C[C@@H]1N=C(c2ccc(S)cc2)c2cc(OC)ccc2-n2c(C)nnc21]

A solution of 2-((4S)-6-(4-bromophenyl)-8-methoxy-1-methyl-4H-benzo[f][1,2,4]triazolo[4,3-a][1,4]diazepin-4-yl)-N-ethylacetamide (100 mg, 0.231 mmol.) in toluene (5 mL), isopropyl alcohol (2 mL) and water (0.5 mL) was charged with sodium-tert butoxide (33 mg, 0.346 mmol)) and stirred at rt for 10 minutes. This solution was charged with a pre-prepared solution of palladium acetate (20 mg, 20% w/w.) and Josiphos (10 mg, 10% w/w.) in toluene (5 mL) then charged with sodium thiosulphate (67 mg, 0.427 mmol) and was heated at 90° C. for 5 h. The reaction mixture was poured over a suspension of zinc powder (100 mg) and (10 mL) 1N HCl solution at 0° C. and stirred for 1 h at 0-10° C. The reaction mixture was partitioned between DCM and H₂O and the aqueous layer was re-extracted with DCM (3 x 10 mL) and the combined organic fractions were dried over anhydrous Na₂SO₄, filtered and concentrated in vacuo resulting in a crude product which was purified by preparative TLC resulting in 25 mg, 27.7% yield of the title compound as a light yellow solid. ¹H NMR (400 MHz, DMSO-d₆) δ 8.20 (s, 1H), 7.78 (d, J=8.9 Hz, 1H), 7.55-7.40 (m, 4H), 7.38 (dd, J=9.0, 2.9 Hz, 1H), 6.87 (d, J=3.0 Hz, 1H), 4.48 (dd, J=8.3, 5.6 Hz, 1H), 3.79 (s, 3H), 3.45-3.40 (m, 1H), 3.30-3.03 (m, 3H), 2.53 (s, 3H), 1.06 (t, J=7.2, Hz, 3H). Mol. Wt: 421.52; MS (ES+): m/z: 421.10 [MH⁺]. HPLC purity: 93.59% (Max plot).

### 2-((4S)-6-(4-bromophenyl)-8-methoxy-1-methyl-4H-benzo[f][1,2,4]triazolo[4,3-a][1,4]diazepin-4-yl)-N-ethylacetamide

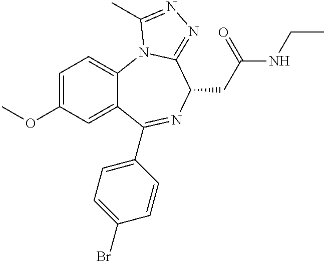 [SMILES: CCNC(=O)C[C@@H]1N=C(c2ccc(Br)cc2)c2cc(OC)ccc2-n2c(C)nnc21]

A solution of 2-((4S)-6-(4-bromophenyl)-8-methoxy-1-methyl-4H-benzo[f][1,2,4]triazolo[4,3-a][1,4]diazepin-4-yl)acetic acid (180 mg, 0.407 mmol) in DCM (18 mL) and DMF (0.1 mL) was cooled to 0° C. and dropwise charged with oxalyl chloride (77 mg, 0.611 mmol) and stirred for 30 min. The resulting suspension was concentrated under reduced pressure resulting in a white solid which was dissolved in THF (5 mL) and cooled at 0° C. then charged with a 2 M solution of ethyl amine (73.5 mg, 1.62 mmol) in THF and stirred at rt for 30 min. The reaction mixture was poured over a cool solution of 1N acetic acid solution at 0° C. then partitioned between DCM and H₂O. The aqueous layer was re-extracted with DCM (3 x 10 mL) and the combined organic fractions were dried over anhydrous Na₂SO₄, filtered and concentrated in vacuo resulting in a crude product which was purified by crystallization in ether resulting in 100 mg, 52.3% yield of the title compound as a white solid. Mol. Wt: 468.35; MS (ES+): m/z: 467.20 [MH⁺], 469.20 [M+2].

### 2-((4S)-6-(4-bromophenyl)-8-methoxy-1-methyl-4H-benzo[f][1,2,4]triazolo[4,3-a][1,4]diazepin-4-yl)acetic acid

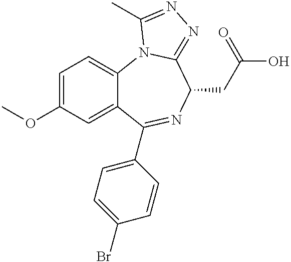 [SMILES: COc1ccc2c(c1)C(c1ccc(Br)cc1)=N[C@@H](CC(=O)O)c1nnc(C)n1-2]

A solution of methyl 2-((4S)-6-(4-bromophenyl)-8-methoxy-1-methyl-4H-benzo[f][1,2,4]triazolo[4,3-a][1,4]diazepin-4-yl)acetate (250 mg, 0.549 mmol) in methanol (10 mL) was charged with lithium hydroxide (65.74 mg, 2.75 mmol) at rt and the reaction mixture was heated at 50° C. for 1 h. The reaction mixture was concentrated in vacuo resulting in a crude product which dissolved in water and acidified with acetic acid resulting in a precipitate which was filtered and washed with water to afford 180 mg, 74.38% yield of the title compound as a white solid. Mol. Wt: 441.28; MS (ES+): m/z: 440.85 [MH⁺], 442.85 [M+2].

### Methyl 2-((4S)-6-(4-bromophenyl)-8-methoxy-1-methyl-4H-benzo[f][1,2,4]triazolo[4,3-a][1,4]diazepin-4-yl)acetate

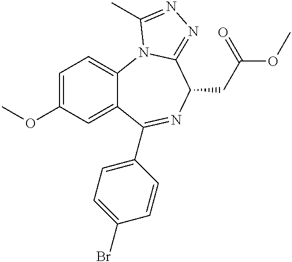

A solution of (S,Z)-methyl 2-(2-(2-acetylhydrazono)-5-(4-bromophenyl)-7-methoxy-2,3-dihydro-1H-benzo[e][1,4]diazepin-3-yl)acetate (500 mg, 1.05 mmol) in THF (5 mL) was charged with acetic acid (5 mL) and the reaction mixture was stirred at rt for 24 h. The reaction mixture was concentrate to dryness under reduced pressure and re-dissolved in DCM followed by the addition of saturated sodium bicarbonate and separated. The aqueous layer was re-extracted with DCM (3×10 mL) and the combined organic fractions were dried over anhydrous Na₂SO₄, filtered and concentrated in vacuo resulting in a crude product which was purified by column chromatography to afford 350 mg, 72.9% yield of the title compound as a white solid. Mol. Wt: 455.30; MS (ES+): m/z: 456.90 [MH⁺], 458.90 [M+2].

### (S,Z)-methyl 2-(2-(2-acetylhydrazono)-5-(4-bromophenyl)-7-methoxy-2,3-dihydro-1H-benzo[e][1,4]diazepin-3-yl)acetate

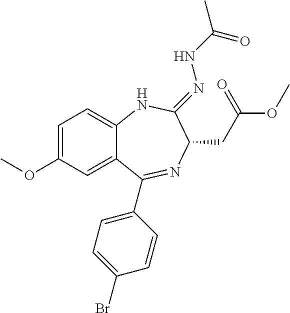

A solution of (S)-methyl 2-(5-(4-bromophenyl)-7-methoxy-2-thioxo-2,3-dihydro-1H-benzo[e][1,4]diazepin-3-yl)acetate (700 mg, 1.61 mmol) in THF (14 mL) was charged with hydrazine hydrate (24.1 mg, 4.83 mmol) and stirred at 10-15° C. for 3 h. This solution was charged with TEA (57 mg, 5.63 mmol) and the reaction mixture was cooled to 0° C. then charged with acetyl chloride (38 mg, 4.83 mmol) and stirred at 0° C. for an additional 30 min. The reaction mixture was diluted with water and DCM and separated. The aqueous layer was re-extracted with DCM (3×10 mL) and the combined organic fractions were dried over anhydrous Na₂SO₄, filtered and concentrated in vacuo resulting in a crude product which was purified by column chromatography resulting in 500 mg, 65.44% yield of the title compound as a white solid. Mol. Wt: 473.32; MS (ES+): m/z: 471.90 [MH⁺], 473.90 [M+2].

### (S)-methyl 2-(5-(4-bromophenyl)-7-methoxy-2-thioxo-2,3-dihydro-1H-benzo[f][1,4]diazepin-3-yl)acetate

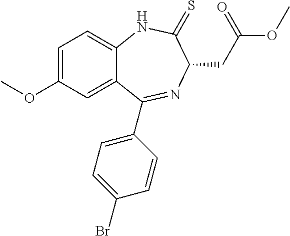

A solution of (5)-methyl 2-(5-(4-bromophenyl)-7-methoxy-2-oxo-2,3-dihydro-1H-benzo[e][1,4]diazepin-3-yl)acetate (1.10 g, 2.63 mmol.) in 1,2-dichloroethane (20 mL) was charged with a suspension of sodium bicarbonate (398 mg, 4.74 mmol) and phosphorus pentasulphite (1.05 g, 4.74 mmol) at rt and the reaction mixture was heated to 60° C. for 5 h. The reaction mixture was filtered through a pad of celite and the filtrate was washed with saturated sodium bicarbonate. The aqueous layer was re-extracted with DCM (3×10 mL) and the combined organic fractions were dried over anhydrous Na₂SO₄, filtered and concentrated in vacuo resulting in a crude product which was purified by column chromatography on silica gel resulting in 900 mg, 78.90% yield of the title compound as a pale yellow solid. Mol. Wt: 433.32; MS (ES+): m/z: 432.80 [MH⁺], 434.80 [M+2].

### (S)-methyl 2-(5-(4-bromophenyl)-7-methoxy-2-oxo-2,3-dihydro-1H-benzo[f][1,4]diazepin-3-yl)acetate

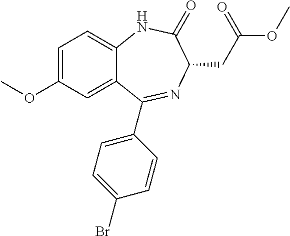

A solution of (5)-methyl 3-((((9H-fluoren-9-yl)methoxy)carbonyl)amino)-4-((2-(4-bromobenzoyl)-4-methoxyphenyl)amino)-4-oxobutanoate (3.20 g, 4.86 mmol) in methanol (48 mL) was charged with TEA (48 mL) and stirred at rt for 48 h. The reaction mixture was concentrated in vacuo to dryness and redissolved in DCM and purified by column chromatography on silica gel resulting in 1.50 g, 73.8% yield of the title compound as a white solid. Mol. Wt: 417.25; MS (ES+): m/z: 416.85 [MH⁺], 418.85 [M+2].

### (S)-methyl 3-((((9H-fluoren-9-yl)methoxy)carbonyl)amino)-4-((2-(4-bromobenzoyl)-4-methoxyphenyl)amino)-4-oxobutanoate

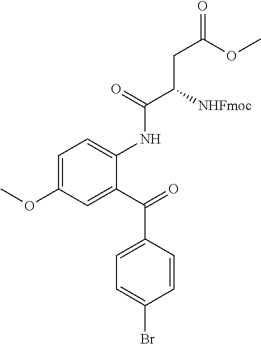

A solution of (2-amino-5-methoxyphenyl)(4-bromophenyl)methanone (2.0 g, 6.53 mmol) in DCM (20 mL) was cooled to 0° C. and charged with sodium bicarbonate (548 mg, 6.53 mmol) followed by addition of N{[(9H-fluoren-9-yl methyl)oxy]carbonyl}-L-alfa aspartyl chloride (2.52 g, 6.53 mmol). The reaction mixture was stirred for 30 minutes at 0° C. then partitioned between water and DCM and H₂O and separated. The aqueous layer was re-extracted with DCM (3×10 mL) and the combined organic fractions were dried over anhydrous Na₂SO₄, filtered and concentrated in vacuo resulting in 4.20 g of title compound as a yellow solid and in the next step without further purification. Mol. Wt: 657.51; MS (ES+): m/z: 657.80 [MH⁺], 657.80 [M+2].

### (2-amino-5-methoxyphenyl)(4-bromophenyl)methanone

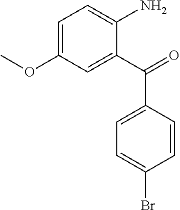 [SMILES: COc1ccc(N)c(C(=O)c2ccc(Br)cc2)c1]

A solution of 6-methoxy-2-methyl-4H-benzo[d][1,3]oxazin-4-one (5 g, 26.15 mmol) in toluene (50 mL) and diethyl ether (25 mL) was charged with a solution of 4-bromophenyl magnesium bromide (5.44 g, 20.92 mmol) at 0° C. then allowed to warm to rt and stirred at rt for 2 h. The reaction mixture was diluted with dil HCl and product was extracted with toluene (3×30 mL). The combined organic fractions were concenrtated under reduced presuure to get a residue which was dissolved in ethanol (20 mL) and con. HCl (20 mL) solution and heated to reflux for 5 h. The reaction mixture was cooled to rt and concentrated in vacuo then partitioned between DCM and 4 N NaOH. The aqueous layer was re-extracted with DCM (3×10 mL) and the combined organic fractions were dried over anhydrous Na₂SO₄, filtered and concentrated in vacuo resulting in a crude product which was purified by column chromatography on silica gel resulting in 4 g, 50% yield of the title compound as a yellow solid. Mol. Wt: 306.15; MS (ES+): m/z: 305.75 [MH⁺], 307.75 [M+2].

### 6-methoxy-2-methyl-4H-benzo[d][1,3]oxazin-4-one

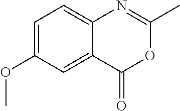

A solution of 2-amino-5-methoxybenzoic acid (10 g, 59.82 mmol.) in acetic anhydride (100 mL) was heated to reflux for 6 h. then concentrated in vacuo. The residue was triturated with diethyl ether and filtered to afford 8 g, 70% yield of the titile compound as a light brown solid. Mol. Wt: 191.18; MS (ES+): m/z: 191.90 [MH⁺].

### 2-amino-5-methoxybenzoic acid

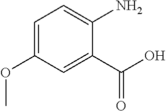

A solution of 2-nitro-5-methoxy benzoic acid (15 g, 76.08 mmol) in ethyl acetate (150 mL) was charged with a suspension of 10% Pd—C (150 mg) and stirred at rt under hydrogen atmosphere for 3 h. The reaction mixture was filtered through a pad of celite and the resulting filtrate concentrated in vacuo to afford 1.14 g, 90% yield of the title compund as an off white solid. Mol. Wt: 167.16; MS (ES+): m/z: 167.90 [MH⁺].

### Example 361

Monomers were purified and characterized according to the procedures described below.

### LIST OF ABBREVIATIONS

HPLC: High performance liquid chromatography
LCMS: Liquid chromatography mass spectrometry
Mm: millimeter
Mm: micron
ml: milliliter
Min: minute
mM: milli molar

Preparative purification of the compounds was performed on Shimadzu preparative HPLC system composed of the following: CBM-20A system controller, LC-8A binary gradient pump, SPD-M20A photodiode array detector, FRC-10A fraction collector, YMC ODS A 500×30 mm×10 μm preparative column using 0.05% (v/v) Trifluoroacetic acid in HPLC grade water (A) and 0.05% (v/v) Trifluoroacetic acid in HPLC grade acetonitrile (B) at a flow rate of 30.0 ml/min and a run time of 40 mins. For basic medium purification, the same instrument was utilized with YMC Triart C18, 500×30 mm×10 μm preparative column using 10 mM Ammonium formate and 0.1%(v/v) liquid ammonia in HPLC grade water (A) and HPLC grade acetonitrile adding 5% (v/v) of mobile phase (A) and 0.1% (v/v) liquid ammonia (B). For both the methods, linear gradient profiles were used depending upon the chromatographic retention and separation of different compounds.

LCMS data was collected on Shimadzu LCMS system equipped with CBM-20A system controller, LC-20AD binary gradient pump, SPD-M20A photodiode array detector, SIL-20AC autosampler, CTO-20AC column oven, LCMS-2010EV single quadrapole mass spectrometer, YMC ODS A 50×4.6 mm×3.0 μm column using 0.05% (v/v) Trifluoroacetic acid in HPLC grade water (A) and 0.05% (v/v) Trifluoroacetic acid in HPLC grade acetonitrile (B) at a flow rate of 1.2 ml/min and a run time of 5.0 mins. The gradient profiles are 20% B to 100% B in 3.0 minute, Hold For 0.5 min, at 3.51 min 20% B Hold till 5.0 min.

All Shimadzu LCMS-2010EV instruments utilized electrospray ionization in positive (ES+) or negative (ES−) ionization mode. The Shimadzu LCMS-2010EV instruments can also be utilized with Atmospheric pressure chemical ionization in positive (AP+) or negative (AP−) ionization mode.

HPLC data was collected on Shimadzu HPLC system equipped with LC-2010 CHT module, SPD-M20A photodiode array detector, YMC ODS A 150×4.6 mm×5.0 μm column using 0.05% (v/v) Trifluoroacetic acid HPLC grade in water (A) and 0.05% (v/v) Trifluoroacetic acid in HPLC grade acetonitrile (B) at a flow rate of 1.4 ml/min and a run time of 15.0 mins. The gradient profiles are 5% B to 95% B in 8.0 min, hold till 9.5 minute, 5% at 11.0 min, and hold till 15.0 mins. For basic medium HPLC, the same instrument was utilized with YMC Triart C18, 150×4.6 mm×5.0 μm column using 10 mM Ammonium formate and 0.1%(v/v) liquid ammonia in HPLC grade water (A) and HPLC grade acetonitrile adding 5% (v/v) of mobile phase (A) and 0.1% (v/v) liquid ammonia (B) at a flow rate of 1.0 ml/min and a run time of 15.0 mins. The gradient profile for basic medium method was 15% B to 95% B in 8.0 min, hold till 9.5 minute, 15% at 13.0 min, and hold till 15.0 mins.

### Example 362

This example demonstrates binding properties of disclosed compounds using a FRET assay.

TR-FRET Assays.

TR-FRET assays were performed as described in Chung et al, J. Med Chem., 2011 54(11): 3827-38 except that FRET was measured on a Spectramax M5 plate reader.

IC₅₀ values determined using the FRET assay:

### BRD-E Monomers:

| IC₅₀ range | Group | Number of monomers |
|---|---|---|
| 1 nM-100 nM | A | BRD-E-01, BRD-E-02, BRD-E-03, |
| | | BRD-E-04, BRD-E-05, BRD-E-06, |
| | | BRD-E-07, BRD-E-07, BRD-E-08, |
| | | BRD-E-09, BRD-E-09, BRD-E-15, |
| | | BRD-E-15, BRD-E-16, BRD-E-16, |
| | | BRD-E-19, BRD-E-19, BRD-E-20, |
| | | BRD-E-21, BRD-E-22 |
| 100 nM-1 μM | B | BRD-E-08, BRD-E-23, BRD-E-24, |
| | | BRD-E-25, BRD-E-26 |

### BRD-N Monomers

| IC₅₀ range | Group | Number of monomers |
|---|---|---|
| 1 nM-100 nM | A | BRD-N-01, BRD-N-02, BRD-N-03, |
| | | BRD-N-05, BRD-N-06, BRD-N-07, |
| | | BRD-N-10, BRD-N-17, BRD-N-21, |
| | | BRD-N-21, BRD-N-23, BRD-N-23, |
| | | BRD-N-25, BRD-N-27, BRD-N-27, |
| | | BRD-N-28, BRD-N-29 |
| 100 nM-1 μM | B | BRD-N-02, BRD-N-03, BRD-N-05, |
| | | BRD-N-07, BRD-N-26, |
| | | BRD-N-28, BRD-N-29 |

### BRD-S Monomers:

| IC₅₀ range (based on | | |
|---|---|---|
| monomer concentration) | Group | Number of monomers |
| 1 nM-100 nM | A | BRD-S-01, BRD-S-02, |
| | | BRD-S-04, BRD-S-05, |
| | | BRD-S-06, BRD-S-07, |
| | | BRD-S-08 |
| 100 nM-300 nM | B | BRD-S-10 |

### Example 363

This example demonstrates binding properties of disclosed compounds using a fluorescence anisotropy assay.

Fluorescence Anisotropy (FA) Binding Assays.

FA assays were performed as described in Chung et al., J. Med Chem., 2011 54(11): 3827-38 except that protein concentrations were 125 nM and plates were read on a Spectramax M5 plate reader.

IC₅₀ values determined using the FA assay:

### BRD-E Monomers:

| | | Number of monomers | | |
|---|---|---|---|---|
| IC₅₀ range | Group | BRD2 | BRD3 | BRD4 |
| 50 nM-1 μM | A | BRD-E-02, BRD-E-01, | BRD-E-10, BRD-E-01, | BRD-E-04, BRD-E-10, |
| | | BRD-E-10, BRD-E-04, | BRD-E-29, BRD-E-02, | BRD-E-01, BRD-E-02, |
| | | BRD-E-29, BRD-E-26, | BRD-E-19, BRD-E-05, | BRD-E-19, BRD-E-29, |
| | | BRD-E-05, BRD-E-20, | BRD-E-22, BRD-E-09, | BRD-E-15, BRD-E-05, |
| | | BRD-E-03, BRD-E-09, | BRD-E-04, BRD-E-20, | BRD-E-22, BRD-E-06, |
| | | BRD-E-22, BRD-E-06, | BRD-E-26, BRD-E-24, | BRD-E-20, BRD-E-09, |
| | | BRD-E-19, BRD-E-31, | BRD-E-08, BRD-E-06, | BRD-E-26, BRD-E-03, |
| | | BRD-E-08, BRD-E-07, | BRD-E-15, BRD-E-38, | BRD-E-08, BRD-E-21, |
| | | BRD-E-14, BRD-E-21, | BRD-E-31, BRD-E-14, | BRD-E-24, BRD-E-38, |
| | | and BRD-E-38 | BRD-E-07, BRD-E-03, | BRD-E-07, BRD-E-31, |
| | | | BRD-E-16, BRD-E-13, | BRD-E-16, BRD-E-14, |
| | | | BRD-E-21 | BRD-E-13 |
| 1 μM-30 μM | B | BRD-E-24, BRD-E-27, | BRD-E-25, BRD-E-23, | BRD-E-25, BRD-E-23, |
| | | BRD-E-15, BRD-E-16, | BRD3-E-27 | BRD-E-27 |
| | | BRD-E-23, BRD-E-25, | | |
| | | BRD-E-13 | | |

### BRD-N Monomers:

| | | Number of monomers | | |
|---|---|---|---|---|
| IC50 range | Group | BRD2 | BRD3 | BRD4 |
| 50 nM-1 μM | A | BRD-N-20, BRD-N-01, | BRD-N-09, BRD-N- | BRD-N-07, BRD-N-01, |
| | | BRD-N-03, BRD-N-07, | 01, BRD-N-07, BRD- | BRD-N-03, BRD-N-09, |
| | | BRD-N-22, BRD-N-24, | N-04, BRD-N-03, | BRD-N-02, BRD-N-20, |
| | | BRD-N-09, BRD-N-04, | BRD-N-02, BRD-N- | BRD-N-10, BRD-N-04, |
| | | BRD-N-30, BRD-N-02, | 30, BRD-N-10, BRD- | BRD-N-22, BRD-N-11, |
| | | BRD-N-23, BRD-N-05, | N-22, BRD-N-24, | BRD-N-30, BRD-N-16, |
| | | BRD-N-08, BRD-N-17, | BRD-N-20, BRD-N- | BRD-N-25, BRD-N-27, |
| | | BRD-N-16, BRD-N-11, | 11, BRD-N-21, BRD- | BRD-N-23, BRD-N-08, |
| | | BRD-N-12, BRD-N-10, | N-27, BRD-N-05, | BRD-N-05, BRD-N-12, |
| | | BRD-N-29, BRD-N-06 | BRD-N-25, BRD-N- | BRD-N-06, BRD-N-21, |
| | | | 08, BRD-N-12, BRD- | BRD-N-17 |
| | | | N-23, BRD-N-28, | |
| | | | BRD-N-16 | |
| 1 μM-30 μM | B | BRD-N-28, BRD-N-27, | BRD-N-06, BRD-N- | BRD-N-28, BRD-N-26, |
| | | BRD-N-26, BRD-N-25, | 17, BRD-N-29, BRD- | BRD-N-29, BRD-N-24 |
| | | BRD-N-21 | N-26 | |

### EQUIVALENTS

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A first monomer capable of forming a biologically useful multimer capable of modulating a protein having a first bromodomain when in contact with a second monomer in an aqueous media, wherein the first monomer is represented by the formula:
X¹—Y¹—Z¹ (Formula I) and pharmaceutically acceptable salts, stereoisomers, metabolites, and hydrates thereof, wherein
X¹ is a first ligand moiety capable of modulating the first bromodomain on said protein;
Y¹ is absent or is a connector moiety covalently bound to X¹ and Z¹;
Z¹ is a first linker capable of binding to the second monomer; and
the second monomer is represented by the formula:
X²—Y²—Z² (Formula II) and pharmaceutically acceptable salts, stereoisomers, metabolites, and hydrates thereof, wherein
X² is a second ligand moiety capable of modulating a second domain on said protein;
Y² is absent or is a connector moiety covalently bound to X² and Z²; and
Z² is a second linker capable of binding to the first monomer through Z¹.

2. The first monomer of claim 1, wherein the protein is independently selected from the group consisting of BRD2, BRD3, BRD4 and BRD-t.

3. The first monomer of claim 1, wherein the protein is a fusion gene product selected from BRD4-NUT or BRD3-NUT.

4. The first monomer of claim 1, wherein the second domain is a second bromodomain.

5. The first monomer of claim 1, wherein the second bromodomain is within 50 Å of the first bromodomain.

6. The first monomer of claim 1, wherein X¹ and X² are independently selected from the group consisting of: 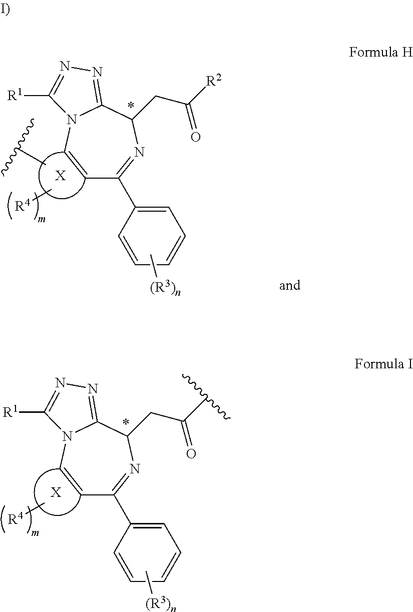
wherein:
X is phenyl, naphthyl, or heteroaryl;
R¹ is C₁₋₃ alkyl, C₁₋₃alkoxy or —S—C₁₋₃ alkyl;
R² is —NR^{2a}R^{2a′} or —OR^{2b}; wherein one of R^{2a} or R^{2a′} is hydrogen, and R^{2b} or the other of R^{2a} or R^{2a′} is selected from the group consisting of C₁₋₆alkyl, haloC₁₋₆alkyl, R^{2c}R^{2c′}N—C₂₋₆ alkyl, carbocyclyl, carbocyclyloC₁₋₄alkyl, heterocyclyl and heterocyclylC₁₋₄alkyl, wherein any of the carbocyclyl or heterocyclyl groups are optionally substituted by one or more substituents selected from the group consisting of halogen, C₁₋₆alkyl, haloC₁₋₆alkyl, C₁₋₆alkoxy, haloC₁₋₆alkoxy, carbonyl, —CO-carbocyclyl, azido, amino, hydroxyl, nitro and cyano, wherein the —CO-carbocyclyl group may be optionally substituted by one or more substituents selected from the group consisting of halogen, C₁₋₆alkyl, haloC₁₋₆alkyl, C₁₋₆alkoxy, haloC₁₋₆alkoxy, azido, nitro and cyano; or
two adjacent groups on any of the carbocyclyl or heterocyclyl groups together with the interconnecting atoms form a 5- or 6-membered ring which ring may contain 1 or 2 heteroatoms independently selected from the group consisting of O, S and N; or R²a and R^{2a′} together with the N atom to which they are attached form a 4-, 5-, 6- or 7-membered ring which optionally contains 1 or 2 heteroatoms independently selected from the group consisting of O, S and N; wherein the 4-, 5-, 6 or 7-membered ring is optionally substituted by C₁₋₆alkyl, hydroxyl or amino;
R^{2c} and R^{2c′} are independently hydrogen or C₁₋₆alkyl;
each R³ is independently selected from the group consisting of hydrogen, hydroxyl, thiol, sulfinyl, amino, halo, C₁₋₆ alkyl, haloC₁₋₆ alkyl, C₁₋₆alkoxy, haloC₁₋₆ alkoxy, nitro, cyano, CF₃, —OCF₃, —COOR⁵, —C₁₋₄alkylamino, phenoxy, benzoxy, and C₁₋₄alkylOH;
each R⁴ is hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆alkyl, nitro, cyano, CF₃, —OCF₃, —COOR⁵; —OS(O)₂C₁₋₄alkyl, phenyl, naphthyl, phenyloxy, benzyloxy or phenylmethoxy, wherein C₁₋₆alkyl, phenyl, and naphthyl are optionally substituted by one two or three substituents selected from the group consisting of hydroxyl, halogen, amino, nitro;
R⁵ is C₁₋₃ alkyl;
* denotes a chiral center;
m is an integer 1 to 3; and
n is an integer 1 to 5; 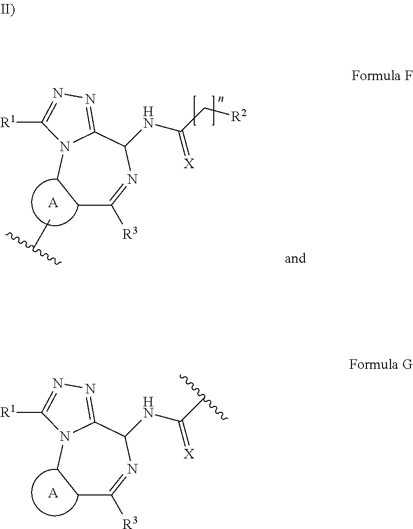
wherein:
X is O or S;
R¹ is C₁₋₆alkyl, haloC₁₋₆alkyl, —(CH₂)—OR^{1a}, or —(CH₂)ₘNR^{1b}R^{1c}; wherein R¹a is hydrogen, C₁₋₆alkyl or haloC₁₋₆alkyl; R^{1b} and R^{1c}, which may be the same or different, are hydrogen, C₁₋₆alkyl or haloC₁₋₆alkyl; and m and n, which may be the same or different, are 1, 2 or 3;
R² is R²a, —OR^{2b}, or —NR^{2c}R^{2d}; wherein R²a and R^{2b} are carbocyclyl, carbocyclylC₁₋₄alkyl, heterocyclyl or heterocyclylC₁₋₄alkyl, or R^{2a} is carbocyclylethenyl or heterocyclylethenyl, wherein any of the carbocyclyl or heterocyclyl groups defined for R^{2a} or R^{2b} are optionally substituted by one or more groups independently selected from the group consisting of halogen, C₁₋₆alkyl, haloC₁₋₆alkyl, C₁₋₆alkoxy, haloC₁₋₆alkoxy, nitro, cyano, dimethylamino, benzoyl and azido; or two adjacent groups on any of the carbocyclyl or heterocyclyl groups defined for R^{2a} or R^{2b} together with the interconnecting atoms form a 5 or 6-membered ring which ring may contain 1 or 2 heteroatoms independently selected from the group consisting of O, S and N; or
R²a and R^{2b} are C₁₋₆alkyl or haloC₁₋₆alkyl; and R^{2c} and R^{2d}, which may be the same or different, are carbocyclyl, carbocyclylC₁₋₄alkyl, heterocyclyl or heterocyclylC₁₋₄alkyl, wherein any of the carbocyclyl or heterocyclyl groups defined for R^{2c} or R^{2d} are optionally substituted by one or more groups independently selected from the group consisting of halogen, C₁₋₆alkyl, haloC₁₋₆alkyl, C₁₋₆alkoxy, haloC₁₋₆alkoxy, nitro, cyano and —CO₂C₁₋₄alkyl; or two adjacent groups on any of the carbocyclyl or heterocyclyl groups defined for R^{2c} and R^{2d} together with the interconnecting atoms form a 5 or 6-membered ring which ring may contain 1 or 2 heteroatoms independently selected from the group consisting of O, S and N; or
R^{2c} and R^{2d} are independently hydrogen, C₁₋₆alkyl or haloC₁₋₆alkyl;
R³ is C₁₋₆alkyl, phenyl, naphthyl, heteroaryl carbocyclyl or heterocyclyl, optionally substituted independently by one or more substitutents selected from the group consisting of halogen, —SR, —S(O)R′, —NHR′, —OR′, C₁₋₆ alkyl, haloC₁₋₆ alkyl, C₁₋₆ alkoxy, haloC₁₋₆ alkoxy, nitro and cyano;
R′ is H or C₁₋₆alkyl;
A is a benzene or aromatic heterocyclic ring, each of which is optionally substituted; and
n is 0, 1 or 2; 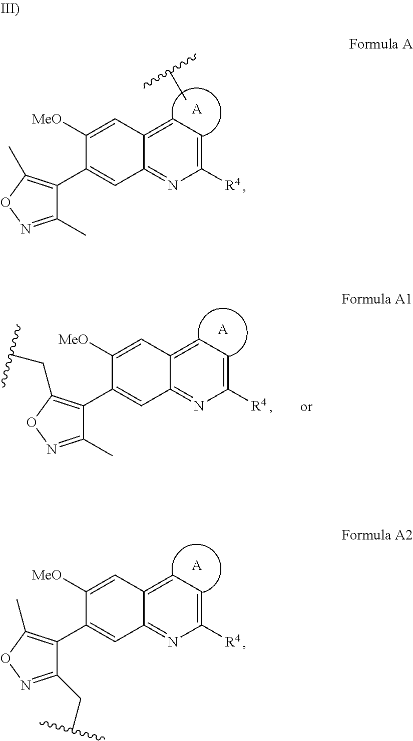
wherein:
R⁴ is hydrogen, cyano or C₁₋₆ alkyl;
A is selected from the group consisting of: 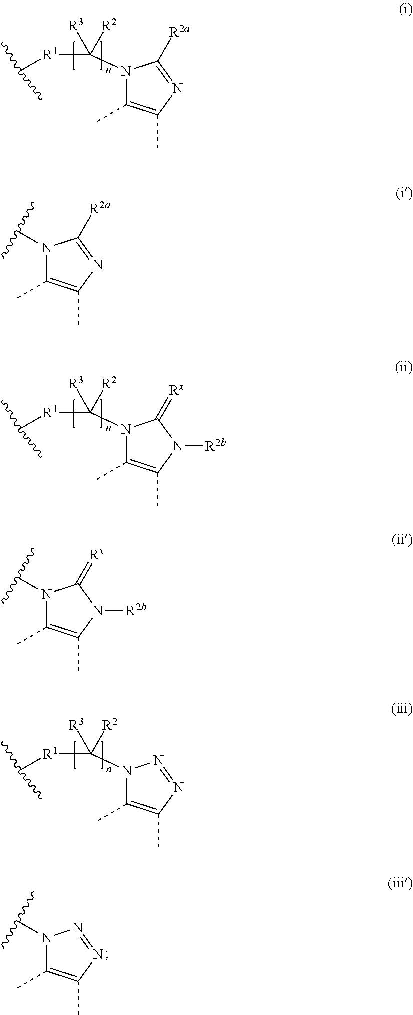
R^{x} is O, NR^{2a}, or S;
R¹ is C₁₋₆alkyl, C₃₋₆cycloalkyl, a 5 or 6 membered heterocyclyl, an aromatic group or a heteroaromatic group, wherein the aromatic group or the heteroaromatic group is optionally substituted by one to three groups selected from the group consisting of halogen, hydroxy, cyano, nitro, C₁₋₆ alkyl, C₁₋₄ alkoxy, haloC₁₋₄ alkyl, haloC₁₋₄ alkoxy, hydroxyC₁₋₄ alkyl, C₁₋₄ alkoxy C₁₋₄ alkyl, C₁₋₄ alkoxycarbonyl, C₁₋₄ alkylsulfonyl, C₁₋₄ alkylsulfonyloxy, C₁₋₄ alkylsulfonyl C₁₋₄ alkyl and C₁₋₄alkylsulfonamido;
R² is hydrogen or C₁₋₆alkyl;
R^{2a} is selected from the group consisting of H, C₁₋₆alkyl, C₁₋₆haloalkyl, (CH₂)ₘcyano, (CH₂)ₘOH, (CH₂)ₘC₁₋₆alkoxy, (CH₂)ₘC₁₋₆haloalkoxy, (CH₂)ₘC₁₋₆haloalkyl, (CH₂)ₘC(O)NR^{a}R^{b}, (CH₂)ₘNR^{a}R^{b} and (CH₂)ₘ C(O)CH₃, (CHR⁶)ₚphenyl optionally substituted by C₁₋₆alkyl, C₁₋₆alkoxy, cyano, halo C₁₋₄alkoxy, haloC₁₋₄alkyl, (CHR⁶)ₚheteroaromatic, (CHR⁶)ₚheterocyclyl; wherein R^{a} is H, C₁₋₆alkyl, or heterocyclyl; wherein R^{b} is H or C₁₋₆alkyl, or
R^{a} and R^{b} together with the N to which they are attached form a 5 or 6 membered heterocyclyl;
R^{2b} is H, C₁₋₆alkyl, (CH₂)₂C₁₋₆alkoxy, (CH₂)₂cyano, (CH₂)ₘphenyl or (CH₂)₂heterocyclyl;
R³ is hydrogen;
R⁶ is hydrogen or C₁₋₆alkyl;
m is 0, 1, 2 or 3;
n is 0, 1 or 2; and
p is 0, 1 or 2; 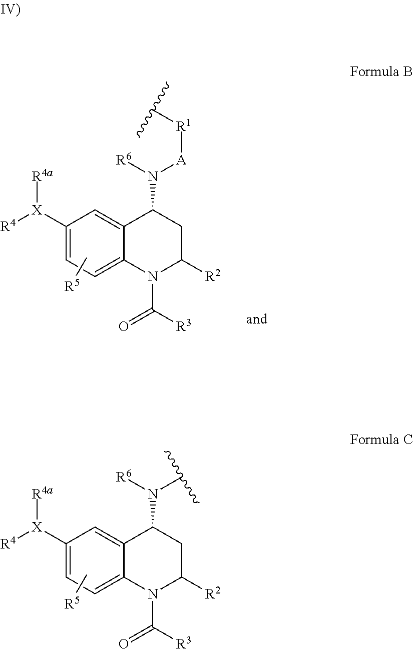
wherein:
A is a bond, C₁₋₄alkyl or —C(O)—;
X is:
i) a 6 to 10 membered aromatic group, or
ii) a 5 to 10 membered heteroaromatic comprising 1, 2 or 3 heteroatoms selected from the group consisting of O, N and S;
R¹ is:
i) phenyl optionally substituted by 1 or 2 substituents independently selected from the group consisting of halogen, cyano, C₁₋₆alkyl, C₁₋₆haloalkyl, C₁₋₆alkoxy, —SO₂C₁₋₆alkyl and —COR⁷,
ii) a 5 to 10 membered heteroaromatic comprising 1, 2 or 3 heteroatoms selected from the group consisting of O, N and S optionally substituted by 1 or 2 substituents independently selected from the group consisting of halogen, cyano, C₁₋₆alkyl, C₁₋₆haloalkyl, C₁₋₆alkoxy and —COR⁷, or
iii) C₁₋₆alkyl, C₀₋₆alkylcyano, C₀₋₆alkylC₁₋₆alkoxy, C₀₋₂alkylC(O)R⁷ or cyclohexyl;
R² is C₁₋₆alkyl;
R³ is C₁₋₆alkyl;
R⁴ is:
i) H, halogen, cyano, C₁₋₆alkyl, C₁₋₆haloalkyl, C₁₋₆alkoxy, C₀₋₆hydroxyalkyl, —SO₂C₁₋₆alkyl, —C(O)NR⁸R⁹, —C(O)R¹°, —C₀₋₆alkyl-NR¹¹R¹², or
ii) —OₘC₁₋₆alkyl substituted by a 5 or 6 membered heterocyclyl or heteroaromatic each comprising 1, 2, 3 or 4 heteroatoms independently selected from the group consisting of N, O and S and wherein said heterocyclyl or heteroaromatic is optionally substituted by 1, 2 or 3 groups independently selected from the group consisting of halogen, cyano, C₁₋₆alkyl, C₁₋₆haloalkyl and C₁₋₆alkoxy, wherein m is 0, 1 or 2, wherein when the heterocyclyl or heteroatomic is linked through a heteroatom and m is 1, then the heteroatom and O are not directly linked if the resultant arrangement would be unstable;
R^{4a} is H, halogen, C₁₋₆alkyl, C₁₋₆haloalkyl, C₁₋₆alkoxy or C₀₋₆hydroxyalkyl;
R⁵ is H, halogen, C₁₋₆alkyl or C₁₋₆alkoxy;
R⁶ is H, C₁₋₆alkyl, C₀₋₆alkylcyano, C₀₋₆alkylC₁₋₆alkoxy or C₀₋₂alkylC(O)R⁷;
R⁷ is hydroxyl, C₁₋₆alkoxy, —NH₂, —NHC₁₋₆alkyl or N(C₁₋₆alkyl)₂;
R⁸ and R⁹ independently are:
i) H, C₁₋₆alkyl, C₀₋₆alkylphenyl, C₀₋₆alkylheteroaromatic, C₃₋₆cycloalkyl, or
ii) R⁸ and R⁹ together with the N to which they are attached form a 5 or 6 membered heterocyclyl or heteroaromatic wherein said heterocyclyl or heteroaromatic may comprise 1, 2 or 3 further heteroatoms independently selected from the group consisting of O, N and S;
R¹⁰ is hydroxyl, C₁₋₆alkoxy or a 5 or 6 membered heterocyclyl or heteroaromatic comprising 1, 2, 3 or 4 heteroatoms selected from the group consisting of O, N and S;
R¹¹ and R¹² independently are:
i) H, C₁₋₆alkyl; or
ii) R¹¹ and R¹² together with the N to which they are attached form a 5 or 6 membered heterocyclyl or heteroaromatic wherein said heterocyclyl or heteroaromatic may comprise 1, 2 or 3 further heteroatoms independently selected from the group consisting of 0, N and S; 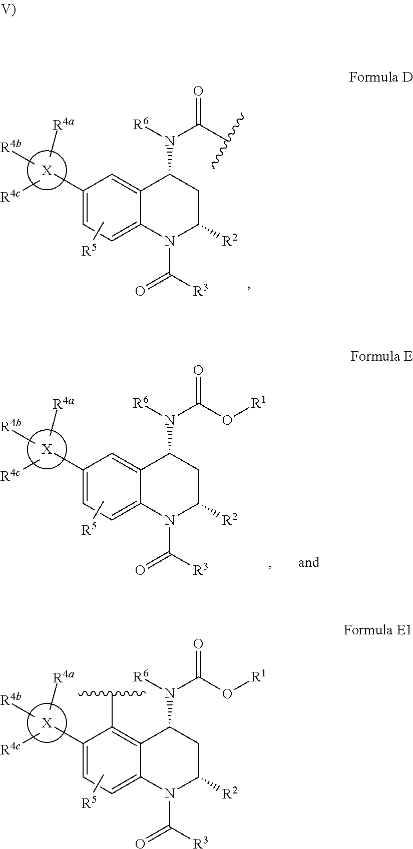
wherein:
R¹ is C₁₋₆alkyl, C₃₋₇cycloalkyl or benzyl;
R² is C₁₋₄alkyl;
R³ is C₁₋₄alkyl;
X is phenyl, naphthyl, or heteroaryl;
R^{4a} is hydrogen, C₁₋₄alkyl or is a group L-Y in which L is a single bond or a C₁₋₆alkylene group and Y is OH, OMe, CO₂H, CO₂C₁₋₆alkyl, CN, or NR⁷R⁸;
R⁷ and R⁸ are independently hydrogen, a heterocyclyl ring, C₁₋₆alkyl optionally substituted by hydroxyl, or a heterocyclyl ring; or
R⁷ and R⁸ combine together to form a heterocyclyl ring optionally substituted by C₁₋₆alkyl, CO₂C₁₋₆alkyl, NH₂, or oxo;
R^{4b} and R^{4c} are independently hydrogen, halogen, C₁₋₆alkyl, or C₁₋₆alkoxy;
R^{4d} is C₁₋₄alkyl or is a group -L-Y— in which L is a single bond or a C₁₋₆alkylene group and Y is —O—, —OCH₂—, —CO₂—, —CO₂C₁₋₆alkyl-, or —N(R⁷)—;
R⁵ is hydrogen, halogen, C₁₋₆alkyl, or C₁₋₆alkoxy;
R⁶ is hydrogen or C₁₋₄alkyl; 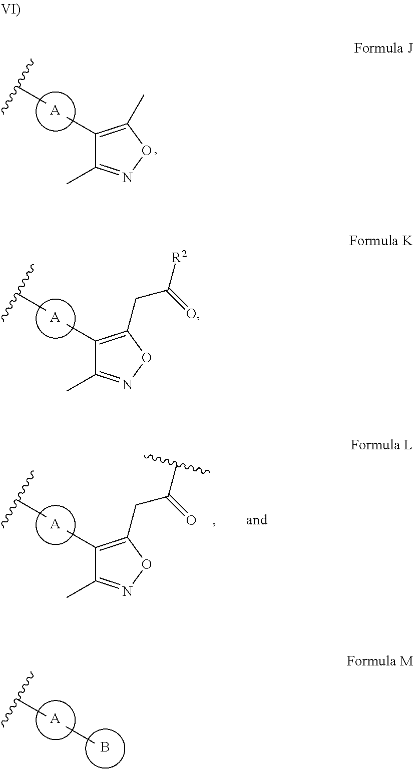
wherein:
A is independently, for each occurrence, a 4-8 membered cycloalkyl, heterocyclic, phenyl, naphthyl, or heteroaryl moiety, each optionally substituted with one, two, three or more R¹ substituents;
R¹ is selected from the group consisting of hydroxy, halogen, oxo, amino, imino, thiol, sulfanylidene, C₁₋₆alkyl, hydroxyC₁₋₆alkyl, —O—C₁₋₆alkyl, —NH—C₁₋₆alkyl, —CO₂H, —C(O)C₁₋₆alkyl, —C(O)O—C₁₋₆alkyl, aminoC₁₋₆ alkyl, haloC₁₋₆ alkyl, —C₁₋₆alkylC(O)R², —O—C(O)R², —NH—C(O)R², —O—C₁₋₆ alkyl-C(O)R², —NHC₁₋₆alkyl-C(O)R², acylaminoC₁₋₆alkyl, nitro, cyano, CF₃, —OCF₃, —OS(O)₂C₁₋₆alkyl, phenyl, naphthyl, phenyloxy, —NH-phenyl, benzyloxy, and phenylmethoxy halogen; wherein C₁₋₆alkyl, phenyl, and naphthyl are optionally substituted by one two or three substituents selected from the group consisting of hydroxyl, halogen, amino, nitro, phenyl and C₁₋₆alkyl; or two R¹ substitutents may be taken together with the atoms to which they are attached to form a fused aliphatic or heterocyclic bicyclic ring system;
R² is —NR^{2a}R^{2a′} or —OR^{2b}; wherein one of R^{2a} or R^{2a′} is hydrogen, and R^{2b} or the other of R^{2a} or R^{2a′} is selected from the group consisting of C₁₋₆alkyl, haloC₁₋₆alkyl, R^{2c}R^{2c′}N—C₂₋₆ alkyl, carbocyclyl, carbocyclyloC₁₋₄alkyl, heterocyclyl and heterocyclylC₁₋₄alkyl, wherein any of the carbocyclyl or heterocyclyl groups are optionally substituted by one or more substituents selected from the group consisting of halogen, C₁₋₆alkyl, haloC₁₋₆alkyl, C₁₋₆alkoxy, haloC₁₋₆alkoxy, carbonyl, —CO-carbocyclyl, azido, amino, hydroxyl, nitro and cyano, wherein the —CO-carbocyclyl group may be optionally substituted by one or more substituents selected from the group consisting of halogen, C₁₋₆alkyl, haloC₁₋₆alkyl, C₁₋₆alkoxy, haloC₁₋₆alkoxy, azido, nitro and cyano; or
two adjacent groups on any of the carbocyclyl or heterocyclyl groups together with the interconnecting atoms form a 5- or 6-membered ring which ring may contain 1 or 2 heteroatoms independently selected from the group consisting of O, S and N; or R^{2a} and R^{2a′} together with the N atom to which they are attached form a 4-, 5-, 6- or 7-membered ring which optionally contains 1 or 2 heteroatoms independently selected from the group consisting of O, S and N; wherein the 4-, 5-, 6 or 7-membered ring is optionally substituted by C₁₋₆alkyl, hydroxyl or amino;
R^{2c} and R^{2c′} are independently hydrogen or C₁₋₆alkyl;
B is selected from the group consisting of 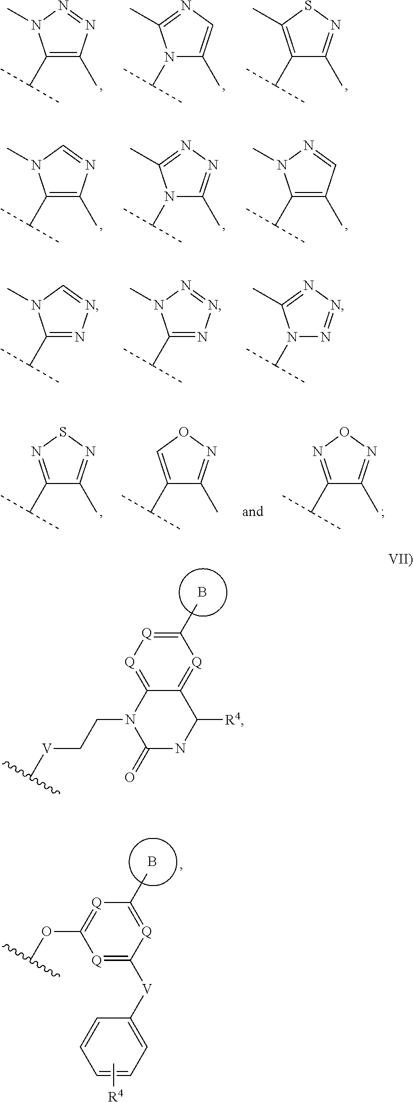 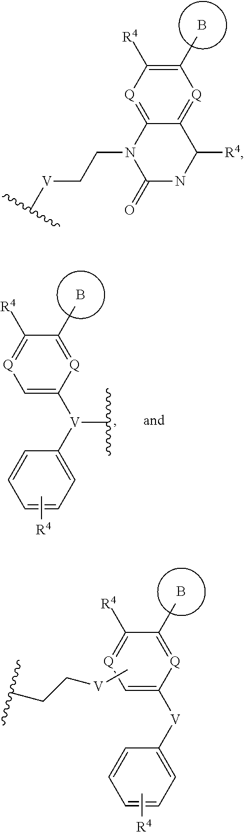
wherein:
B is selected from the group consisting of: 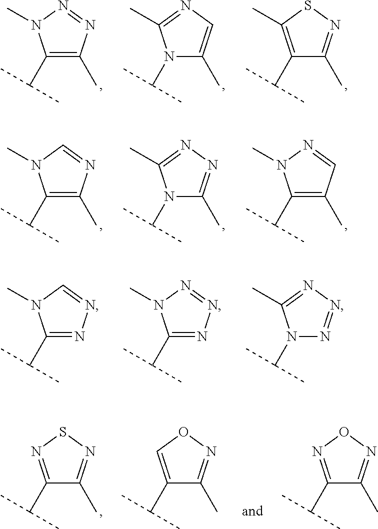
Q is independently, for each occurrence, N or CH;
V is independently, for each occurrence, O, S, NR⁴, or a bond; and
R⁴ is independently selected from the group consisting of hydrogen, hydroxyl, halo, amino, thiol, C₁₋₆ alkyl, haloC₁₋₆ alkyl, C₁₋₆ alkoxy, —NH—C₁₋₆ alkyl, —S—C₁₋₆ alkyl, haloC₁₋₆ alkoxy, nitro, cyano, —CF₃, —OCF₃, —C₁₋₄alkylamino, phenoxy, benzoxy, and C₁₋₄alkylOH; 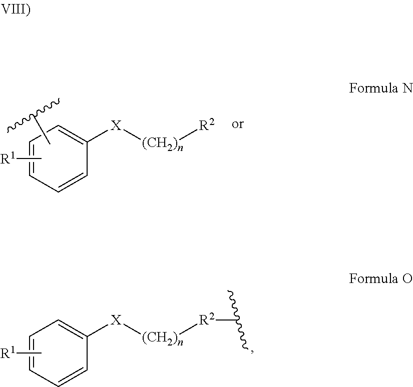
wherein:
R¹ is selected from the group consisting of hydrogen, lower alkyl, phenyl, naphthyl, aralkyl, heteroalkyl, SO₂, NH₂, NO₂, CH₃, CH₂CH₃, OCH₃, OCOCH₃, CH₂COCH₃, OH, CN, and halogen;
R² is selected from the group consisting of hydrogen, lower alkyl, aralkyl, heteroalkyl, phenyl, naphthyl, SO₂, NH₂, NH₃⁺, NO₂, CH₃, CH₂CH₃, OCH₃, OCOCH₃, CH₂COCH₃, OH, halogen, carboxy, and alkoxy;
X is selected from the group consisting of lower alkyl, SO₂, NH, NO₂, CH₃, CH₂CH₃, OCH₃, OCOCH₃, CH₂COCH₃, OH, carboxy, and alkoxy; and
n is an integer from 0 to 10; 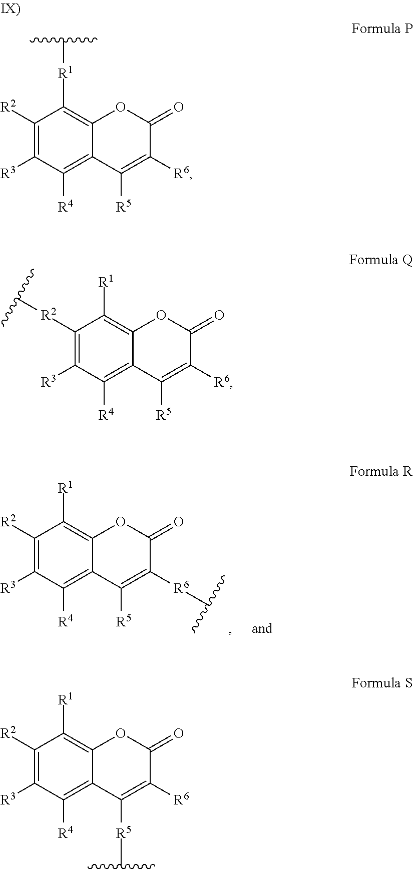
wherein:
R¹, R², R³, R⁴, R⁵, and R⁶ are independently selected from the group consisting of hydrogen, lower alkyl, phenyl, naphthyl, aralkyl, heteroaryl, SO₂, NH₂, NH₃⁺, NO², SO², CH³, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂OH, NH(CH₂)₃CH₃, NHCH₃, SH, halogen, carboxy, and alkoxy; 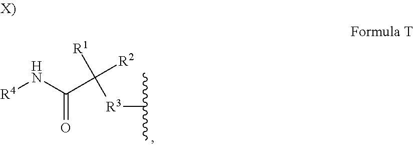
wherein:
R¹, R², and R³ are independently selected from the group consisting of hydrogen, lower alkyl, phenyl, naphthyl, aralkyl, heteroaryl, SO₂, NH₂, NH₃⁺, NO₂, SO₂, CH₃, CH₂CH₃, OCH₃, OCOCH₃, CH₂COCH₃, OH, SH, halogen, carboxy, and alkoxy; R⁴ is selected from the group consisting of lower alkyl, phenyl, naphthyl, SO₂, NH, NO₂, CH₃, CH₂CH₃, OCH₃, OCOCH₃, CH₂COCH₃, OH, carboxy, and alkoxy; 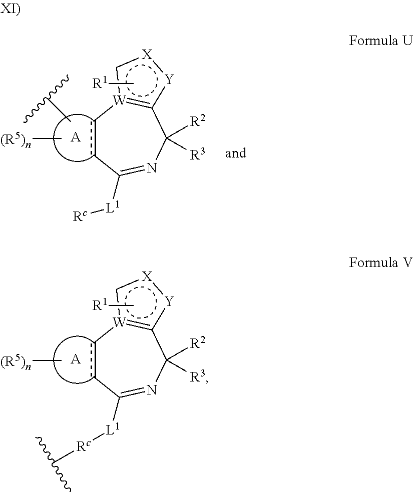
or a pharmaceutically acceptable salt thereof,
wherein:
X is O or N;
Y is O or N; wherein at least one of X or Y is O;
W is C or N;
R¹ is H, alkyl, alkenyl, alkynyl, aralkyl, phenyl, naphthyl, heteroaryl, halo, CN, OR^{A}, NR^{A}R^{B}, N(R^{A})S(O)_{q}R^{A}R^{B}, N(R^{A})C(O)R^{B}, N(R^{A})C(O)NR^{A}R^{B}, N(R^{A})C(O)OR^{A}, N(R^{A})C(S)NR^{A}R^{B}, S(O)_{q}R^{A}, C(O)R^{A}, C(O)OR^{A}, OC(O)R^{A}, or C(O)NR^{A}R^{B};
each R^{A} is independently alkyl, alkenyl, or alkynyl, each containing 0, 1, 2, or 3 heteroatoms selected from O, S, or N; phenyl; naphthyl, heteroaryl; heterocyclic; carbocyclic; or hydrogen;
each R^{B} is independently alkyl, alkenyl, or alkynyl, each containing 0, 1, 2, or 3 heteroatoms selected from O, S, or N; phenyl; naphthyl; heteroaryl; heterocyclic; carbocyclic; or hydrogen; or
R^{A} and R^{B}, together with the atoms to which each is attached, can form a heterocycloalkyl or a heteroaryl; each of which is optionally substituted;
Ring A is cycloalkyl, phenyl, naphthyl, heterocycloalkyl, or heteroaryl;
R^{C} is alkyl, alkenyl, alkynyl, cycloalkyl, phenyl, naphthyl, heterocycloalkyl, or heteroaryl, each optionally substituted with 1-5 independently selected R⁴, and when L¹ is other than a covalent bond, R^{C} is additionally selected from H;
R² and R³ are each independently H, halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″), or —(CH₂)ₚR^{x}; or
R₂ and R₃ together with the atoms to which each is attached, form an optionally substituted 3-7 membered saturated or unsaturated spiro-fused ring having 0-3 heteroatoms independently selected from nitrogen, oxygen, or sulfur;
each R^{x} is independently halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R)SO₂R, —N(R)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″);
L¹ is a covalent bond or an optionally substituted bivalent C₁₋₆ hydrocarbon chain wherein one or two methylene units is optionally replaced by —NR′—, —N(R′)C(O)—, —C(O)N(R′)—, —N(R′)SO₂—, —SO₂N(R′)— —O—, —C(O)—, —OC(O)—, —C(O)O—, —S—, —SO— or —SO₂—;
each R is independently hydrogen, alkyl, alkenyl, alkynyl, phenyl, naphthyl, aralkyl, cycloalkyl, heteroaryl, or heterocycloalkyl;
each R′ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an heteroaryl or heterocycloalkyl group; each R″ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an heteroaryl or heterocycloalkyl group; or
R′ and R″, together with the atoms to which each is attached, can form cycloalkyl, heterocycloalkyl, phenyl, naphthyl, or heteroaryl; each of which is optionally substituted;
each R⁴ is independently alkyl, alkenyl, alkynyl, phenyl, naphthyl, aralkyl, cycloalkyl, heteroaryl, or heterocycloalkyl, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R″))N(R′)(R″), —C═NN(R′)(R″), —C═NOR, —C(═N(R′))N(R′)(R″), —OC(O)R, or —OC(O)N(R′)(R″);
each R⁵ is independently —R, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);
n is 0-5;
each q is independently 0, 1, or 2; and
p is 1-6; 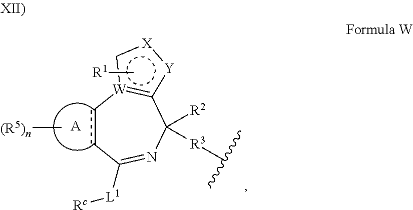
wherein:
X is O or N;
Y is O or N; wherein at least one of X or Y is O;
W is C or N;
R¹ is H, alkyl, alkenyl, alkynyl, aralkyl, phenyl, naphthyl, heteroaryl, halo, CN, OR^{A}, NR^{A}R^{B}, N(R^{A})S(O)_{q}R^{A}R^{B}, N(R^{A})C(O)R^{B}, N(R^{A})C(O)NR^{A}R^{B}, N(R^{A})C(O)OR^{A}, N(R^{A})C(S)NR^{A}R^{B}, S(O)_{q}R^{A}, C(O)R^{A}, C(O)OR^{A}, OC(O)R^{A}, or C(O)NR^{A}R^{B};
each R^{A} is independently optionally substituted alkyl, optionally substituted alkenyl or optionally substituted alkynyl, each containing 0, 1, 2, or 3 heteroatoms selected from O, S, or N; phenyl; naphthyl; heteroaryl; heterocyclic; carbocyclic; or hydrogen;
each R^{B} is independently alkyl, alkenyl, or alkynyl, each containing 0, 1, 2, or 3 heteroatoms selected from O, S, or N; phenyl; naphthyl; heteroaryl; heterocyclic; carbocyclic; or hydrogen; or
R^{A} and R^{B}, together with the atoms to which each is attached, can form a heterocycloalkyl or a heteroaryl; each of which is optionally substituted;
Ring A is cycloalkyl, phenyl, naphthyl, heterocycloalkyl, or heteroaryl;
R^{C} is alkyl, alkenyl, alkynyl, cycloalkyl, phenyl, naphthyl, heterocycloalkyl, or heteroaryl, each optionally substituted with 1-5 independently selected R⁴, and when L¹ is other than a covalent bond, R^{C} is additionally selected from H;
R² is H, halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″), or —(CH₂)ₚR^{x};
R³ is a bond or optionally substituted alkyl; or
R₂ and R₃ together with the atoms to which each is attached, form an optionally substituted 3-7 membered saturated or unsaturated spiro-fused ring having 0-3 heteroatoms independently selected from nitrogen, oxygen, or sulfur;
each R^{x} is independently halogen, alkyl, alkenyl, alkynyl, phenyl, naphthyl, aralkyl, cycloalkyl, heteroaryl, heterocycloalkyl, —OR, —SR, —CN, —N(R′)(R″), —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R)SO₂R, —N(R)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, —OC(O)N(R′)(R″);
L¹ is a covalent bond or an optionally substituted bivalent C₁₋₆ hydrocarbon chain wherein one or two methylene units is optionally replaced by —NR′—, —N(R′)C(O)—, —C(O)N(R′)—, —N(R′)SO₂—, —SO₂N(R′)—, —O—, —C(O)—, —OC(O)—, —C(O)O—, —S—, —SO—, or —SO₂—;
each R is independently hydrogen, alkyl, alkenyl, alkynyl, phenyl, naphthyl, aralkyl, cycloalkyl, heteroaryl, or heterocycloalkyl;
each R′ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an heteroaryl or heterocycloalkyl group; each R″ is independently —R, —C(O)R, —C(S)R, —CO₂R, —C(O)N(R)₂, —C(S)N(R)₂, —S(O)R, —SO₂R, —SO₂N(R)₂, or two R groups on the same nitrogen are taken together with their intervening atoms to form an optionally substituted heteroaryl or heterocycloalkyl group; or
R′ and R″, together with the atoms to which each is attached, can form cycloalkyl, heterocycloalkyl, phenyl, naphthyl, or heteroaryl; each of which is optionally substituted;
each R⁴ is independently alkyl, alkenyl, alkynyl, phenyl, naphthyl, aralkyl, cycloalkyl, heteroaryl, or heterocycloalkyl, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);
each R⁵ is independently —R, halogen, —OR, —SR, —N(R′)(R″), —CN, —NO₂, —C(O)R, —C(S)R, —CO₂R, —C(O)N(R′)(R″), —C(O)SR, —C(O)C(O)R, —C(O)CH₂C(O)R, —C(S)N(R′)(R″), —C(S)OR, —S(O)R, —SO₂R, —SO₂N(R′)(R″), —N(R′)C(O)R, —N(R′)C(O)N(R′)(R″), —N(R′)C(S)N(R′)(R″), —N(R′)SO₂R, —N(R′)SO₂N(R′)(R″), —N(R′)N(R′)(R″), —N(R′)C(═N(R′))N(R′)(R″), —C═NN(R′)(R″), —C═NOR, —C(═N(R′))N(R′)(R″), —OC(O)R, or —OC(O)N(R′)(R″);
n is 0-5;
each q is independently 0, 1, or 2; and
p is 1-6; 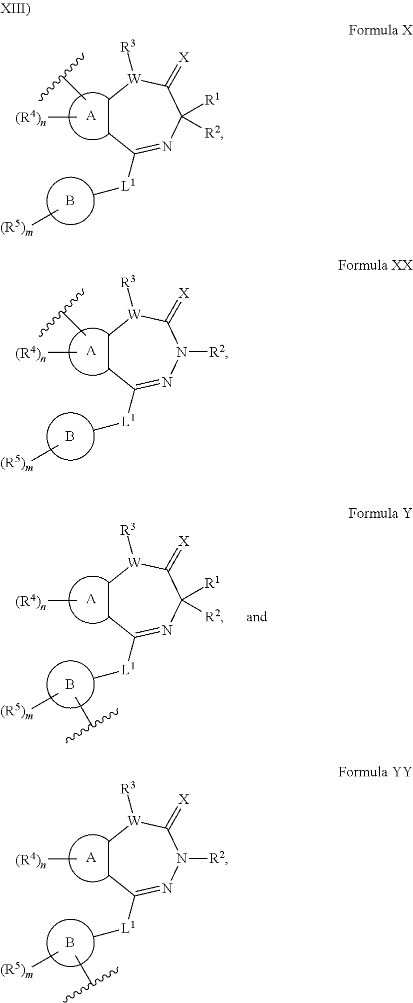
wherein:
Ring A is benzo, or a 5-6 membered fused heteroaryl ring having 1-3 heteroatoms independently selected from nitrogen, oxygen, or sulfur;
Ring B is a 3-7 membered saturated or partially unsaturated carbocyclic ring, phenyl, an 8-10 membered bicyclic saturated, partially unsaturated, phenyl or naphthyl ring, a 4-7 membered saturated or partially unsaturated heterocyclic ring having 1-2 heteroatoms independently selected from nitrogen, oxygen, and sulfur, a 5-6 membered monocyclic heteroaryl ring having 1-3 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
L¹ is a covalent bond or an optionally substituted bivalent C₁₋₆ hydrocarbon chain wherein one or two methylene units is optionally replaced by —NR′—, —N(R′)C(O)—, —C(O)N(R′), —N(R′)SO₂—, —SO₂N(R′), —O—, —C(O)—, —OC(O)—, —C(O)O—, —S—, —SO— or —SO₂—;
R¹ is hydrogen, halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂, or —(CH₂)ₚR^{x};
p is 0-3;
R^{x} is halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R′))N(R′)₂, —OC(O)R, —OC(O)N(R′)₂;
R² is hydrogen, halogen, —CN, —SR, or optionally substituted C₁₋₆ aliphatic, or:
R¹ and R² are taken together with their intervening atoms to form an optionally substituted 3-7 membered saturated or partially unsaturated spiro-fused ring having 0-2 heteroatoms independently selected from nitrogen, oxygen, or sulfur;
each R is independently hydrogen or an optionally substituted group selected from C₁₋₆ aliphatic, phenyl, a 3-7 membered saturated or partially unsaturated carbocyclic ring, a 7-10 membered bicyclic saturated, partially unsaturated, phenyl or naphthyl ring, a 5-6 membered monocyclic heteroaryl ring having 1-3 heteroatoms independently selected from nitrogen, oxygen, and sulfur, a 4-7 membered saturated or partially unsaturated heterocyclic ring having 1-2 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
each R′ is independently —R, —C(O)R, —C(S)R, —CO₂R, —C(O)N(R)₂, —C(S)N(R)₂, —S(O)R, —SO₂R, —SO₂N(R)₂, or two R′ on the same nitrogen are taken together with their intervening atoms to form an optionally substituted group selected from a 4-7 membered monocyclic saturated or partially unsaturated ring having 1-2 heteroatoms independently selected from nitrogen, oxygen, and sulfur, or a 7-12 membered bicyclic saturated, partially unsaturated, or aromatic fused ring having 1-3 heteroatoms independently selected from nitrogen, oxygen, and sulfur;
W is 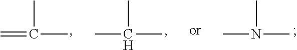
R³ is optionally substituted C₁₋₆ aliphatic;
X is oxygen or sulfur, or:
R³ and X are taken together with their intervening atoms to form an optionally substituted 5-membered heteroaryl ring having 1-4 heteroatoms independently selected from nitrogen, oxygen, or sulfur;
each of m and n is independently 0-4, as valency permits; and
each of R⁴ and R⁵ is independently —R, halogen, —OR, —SR, —N(R′)₂, —CN, —NO₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, or —OC(O)N(R′)₂; 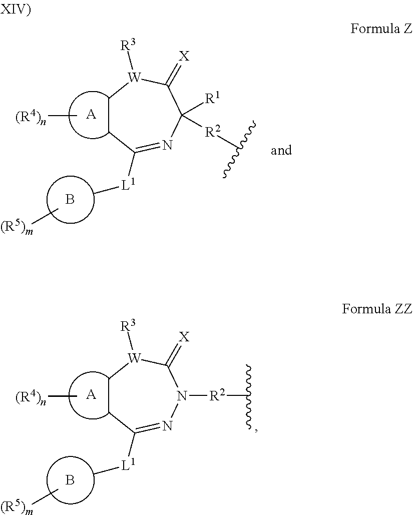
wherein:
Ring A is benzo, or a 5-6 membered fused heteroaryl ring having 1-3 heteroatoms independently selected from nitrogen, oxygen, or sulfur;
Ring B is a 3-7 membered saturated or partially unsaturated carbocyclic ring, phenyl, an 8-10 membered bicyclic saturated, partially unsaturated, phenyl or naphthyl ring, a 4-7 membered saturated or partially unsaturated heterocyclic ring having 1-2 heteroatoms independently selected from nitrogen, oxygen, and sulfur, a 5-6 membered monocyclic heteroaryl ring having 1-3 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
L¹ is a covalent bond or an optionally substituted bivalent C₁₋₆ hydrocarbon chain wherein one or two methylene units is optionally replaced by —NR′—, —N(R′)C(O)—, —C(O)N(R′), —N(R′)SO₂—, —SO₂N(R′), —O—, —C(O)—, —OC(O)—, —C(O)O—, —S—, —SO— or —SO₂—;
R¹ is hydrogen, halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂, or —(CH₂)ₚR^{x};
p is 0-3;
R^{x} is halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂;
R² is a bond or optionally substituted C₁₋₆ aliphatic, or:
R¹ and R² are taken together with their intervening atoms to form an optionally substituted 3-7 membered saturated or partially unsaturated spiro-fused ring having 0-2 heteroatoms independently selected from nitrogen, oxygen, or sulfur;
each R is independently hydrogen or an optionally substituted group selected from C₁₋₆ aliphatic, phenyl, a 3-7 membered saturated or partially unsaturated carbocyclic ring, a 7-10 membered bicyclic saturated, partially unsaturated, phenyl, or naphthyl ring, a 5-6 membered monocyclic heteroaryl ring having 1-3 heteroatoms independently selected from nitrogen, oxygen, and sulfur, a 4-7 membered saturated or partially unsaturated heterocyclic ring having 1-2 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
each R′ is independently —R, —C(O)R, —C(S)R, —CO₂R, —C(O)N(R)₂, —C(S)N(R)₂, —S(O)R, —SO₂R, —SO₂N(R)₂, or two R′ on the same nitrogen are taken together with their intervening atoms to form an optionally substituted group selected from a 4-7 membered monocyclic saturated or partially unsaturated ring having 1-2 heteroatoms independently selected from nitrogen, oxygen, and sulfur, or a 7-12 membered bicyclic saturated, partially unsaturated, or aromatic fused ring having 1-3 heteroatoms independently selected from nitrogen, oxygen, and sulfur;
W is 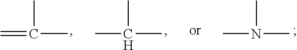
R³ is optionally substituted C₁₋₆ aliphatic;
X is oxygen or sulfur, or:
R³ and X are taken together with their intervening atoms to form an optionally substituted 5-membered heteroaryl ring having 1-4 heteroatoms independently selected from nitrogen, oxygen, or sulfur;
each of m and n is independently 0-4, as valency permits; and
each of R⁴ and R⁵ is independently —R, halogen, —OR, —SR, —N(R′)₂, —CN, —NO₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, or —OC(O)N(R′)₂; 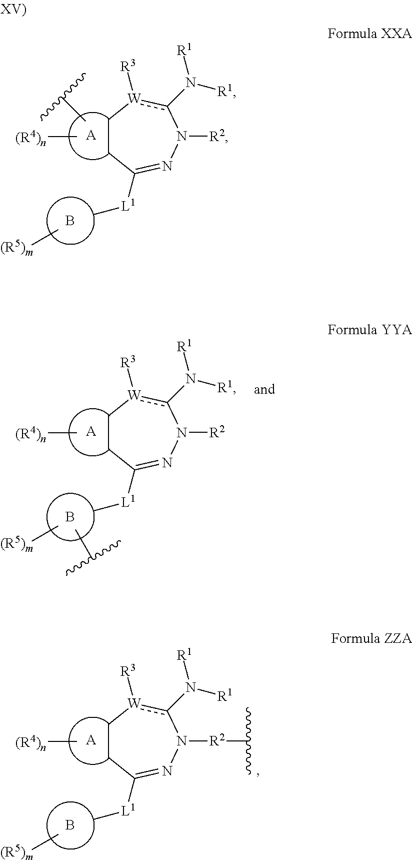
wherein:
Ring A is benzo, or a 5-6 membered fused heteroaryl ring having 1-3 heteroatoms independently selected from nitrogen, oxygen, or sulfur;
Ring B is a 3-7 membered saturated or partially unsaturated carbocyclic ring, phenyl, an 8-10 membered bicyclic saturated, partially unsaturated, phenyl, or naphthyl ring, a 4-7 membered saturated or partially unsaturated heterocyclic ring having 1-2 heteroatoms independently selected from nitrogen, oxygen, and sulfur, a 5-6 membered monocyclic heteroaryl ring having 1-3 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
L¹ is a covalent bond or an optionally substituted bivalent C₁₋₆ hydrocarbon chain wherein one or two methylene units is optionally replaced by —NR′—, —N(R′)C(O)—, —C(O)N(R′), —N(R′)SO₂—, —SO₂N(R′), —O—, —C(O)—, —OC(O)—, —C(O)O—, —S—, —SO— or —SO₂—;
R¹ is independently hydrogen, halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R)SO₂N(R)₂, —N(R′)C(═N(R′))N(R′)₂, —C═NN(R′)₂, —C═NOR, —C(═N(R′))N(R′)₂, —OC(O)R, —OC(O)N(R′)₂, or —(CH₂)ₚR^{x};
p is 0-3;
R^{x} is halogen, optionally substituted C₁₋₆ aliphatic, —OR, —SR, —CN, —N(R′)₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, —OC(O)N(R′)₂;
R² is a bond, hydrogen, or optionally substituted C₁₋₆ aliphatic;
each R is independently hydrogen or an optionally substituted group selected from C₁₋₆ aliphatic, phenyl, a 3-7 membered saturated or partially unsaturated carbocyclic ring, a 7-10 membered bicyclic saturated, partially unsaturated, phenyl, or naphthyl ring, a 5-6 membered monocyclic heteroaryl ring having 1-3 heteroatoms independently selected from nitrogen, oxygen, and sulfur, a 4-7 membered saturated or partially unsaturated heterocyclic ring having 1-2 heteroatoms independently selected from nitrogen, oxygen, and sulfur, a 7-10 membered bicyclic saturated or partially unsaturated heterocyclic ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
each R′ is independently —R, —C(O)R, —C(S)R, —CO₂R, —C(O)N(R)₂, —C(S)N(R)₂, —S(O)R, —SO₂R, —SO₂N(R)₂, or two R′ on the same nitrogen are taken together with their intervening atoms to form an optionally substituted group selected from a 4-7 membered monocyclic saturated or partially unsaturated ring having 1-2 heteroatoms independently selected from nitrogen, oxygen, and sulfur, or a 7-12 membered bicyclic saturated, partially unsaturated, or aromatic fused ring having 1-3 heteroatoms independently selected from nitrogen, oxygen, and sulfur;
W is C or N;
R³ is optionally substituted C₁₋₆ aliphatic; [symbol]
is a single or double bond;
each of m and n is independently 0-4, as valency permits; and
each of R⁴ and R⁵ is independently —R, halogen, —OR, —SR, —N(R′)₂, —CN, —NO₂, —C(O)R, —C(S)R, —CO₂R, —C(O)N(R′)₂, —C(O)SR, —C(O)C(O)R, —C(O)CH₂C(O)R, —C(S)N(R′)₂, —C(S)OR, —S(O)R, —SO₂R, —SO₂N(R′)₂, —N(R′)C(O)R, —N(R′)C(O)N(R′)₂, —N(R′)C(S)N(R′)₂, —N(R′)SO₂R, —N(R′)SO₂N(R′)₂, —N(R′)N(R′)₂, —N(R′)C(═N(R′))N(R′)₂, —C═NN(R)₂, —C═NOR, —C(═N(R))N(R′)₂, —OC(O)R, or —OC(O)N(R′)₂; 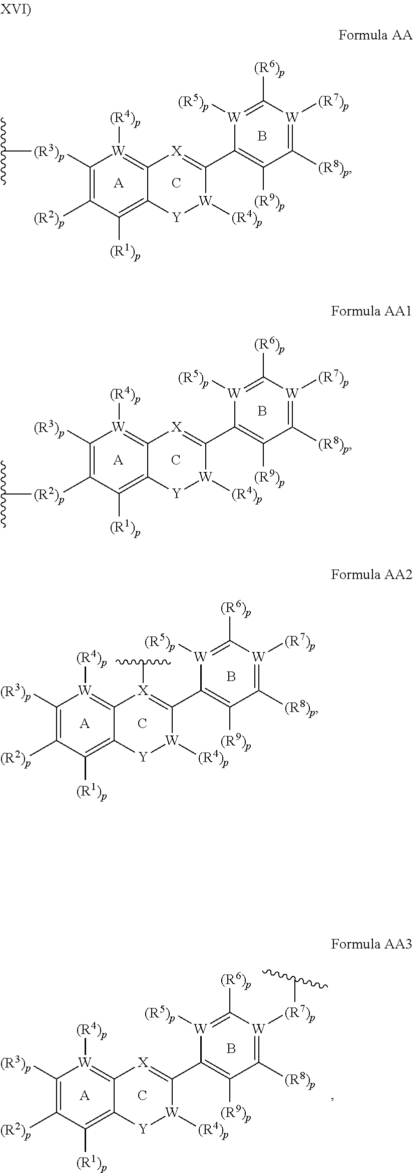
wherein:
X is selected from N and CH;
Y is CO;
R¹ and R³ are each independently selected from alkoxy and hydrogen;
R² is selected from alkoxy, alkyl, and hydrogen;
R⁶ and R⁸ are each independently selected from alkyl, alkoxy, chloride, and hydrogen;
R⁵ and R⁹ are each hydrogen;
R⁷ is selected from amino, hydroxyl, alkoxy, and alkyl substituted with a heterocyclyl;
R¹⁰ is hydrogen; or
two adjacent substituents selected from R⁶, R⁷, and R⁸ are connected to form a heterocyclyl;
each W is independently selected from C and N, wherein if W is N, then p is 0 or 1, and if W is C, then p is 1;
for W—(R¹⁰)ₚ, W is N and p is 1; and
for W—(R⁴)ₚ, W is C, p is 1 and R⁴ is H, or W is N and p is 0; 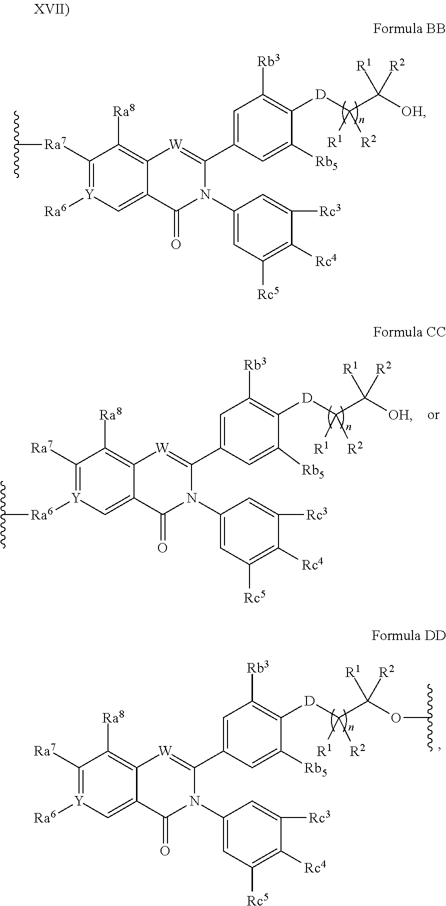
wherein:
Y and W are each independently selected from carbon and nitrogen;
Ra⁶ is selected from fluoride, hydrogen, C₁-C₃ alkoxy, cyclopropyloxy, SO₂R₃, SOR₃, and SR₃, wherein if Y is nitrogen then Ra⁶ is absent;
Ra⁷ is selected from hydrogen, fluoride, SO₂R₃, SOR₃, and SR₃;
Ra⁸ is selected from hydrogen, C₁-C₃ alkoxy, cyclopropyloxy, chloride, and bromide;
n is selected from 1, 2, or 3;
D is selected from O, NH, NR₁, S, or C;
Rb³ and Rb⁵ are independently selected from hydrogen and C₁-C₃ alkyl;
R_{C}³ and R_{C}⁵ are independently selected from hydrogen, C₁-C₃ alkyl, and cyclopropyl;
R_{C}⁴ is selected from F, Cl, Br, I, CF₃, C₁-C₆ alkyl, C₃-C₆ cycloalkyl, NHC(O)R⁴, NHSO₂R⁴, C(O)OR⁴, and 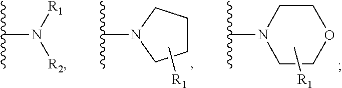
R¹, R′¹, R² and R′² are independently selected from hydrogen, fluoride, C₁-C₃ alkyl, and cyclopropyl, wherein R¹ and R² and/or R′¹ and R′² may be connected to form a 3-6 membered ring;
R³ is selected from C₁-C₃ alkyl and cyclopropyl; and
R⁴ is selected from hydrogen, C₁-C₄ alkyl, C₃-C₅ cycloalkyl, phenyl, and naphthyl, provided that if Ra⁷ or Ra⁶ is fluoride, then R_{C}⁴ is not bromide; 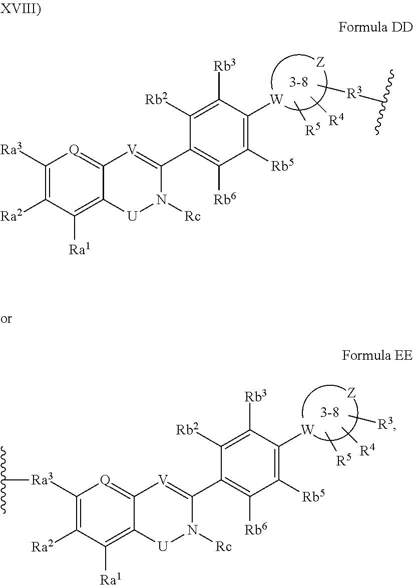
wherein:
Q and V are independently selected from CH and nitrogen;
U is selected from C═O, C═S, SO₂, S═O, SR′, CR′R², CR¹OR², CR¹SR²;
R¹ and R² are independently selected from hydrogen and C₁-C₆ alkyl;
Rc is selected from hydrogen, C₁-C₆ alkyl, and C₃-C₆ cycloalkyl;
Ra¹, Ra², and Ra³ are independently selected from hydrogen, C₁-C₆ alkyl, C₁-C₆ alkenyl, C₁-C₆ alkynyl, C₁-C₆ alkoxy, halogen, amino, amide, hydroxyl, heterocycle, and C₃-C₆ cycloalkyl, wherein Ra¹ and Ra² and/or Ra² and Ra³ may be connected to form a cycloalkyl or a heterocycle;
Rb² and Rb⁶ are independently selected from hydrogen, halogen, C₁-C₆ alkyl, C₁-C₆ alkenyl, C₃-C₆ cycloalkyl, hydroxyl, and amino;
Rb³ and Rb⁵ are independently selected from hydrogen, halogen, C₁-C₆ alkyl, C₁-C₆ alkoxy, C₃-C₆ cycloalkyl, hydroxyl, and amino, wherein Rb² and Rb³ and/or Rb⁵ and Rb⁶ may be connected to form a cycloalkyl or a heterocycle; 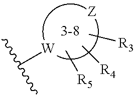
represents a 3-8 membered ring system wherein: W is selected from carbon and nitrogen; Z is selected from CR⁶R⁷, NR⁸, oxygen, sulfur, —S(O)—, and —SO₂—;
said ring system being optionally fused to another ring selected from cycloalkyl, heterocycle, and phenyl, and wherein said ring system is optionally selected from rings having the structures: 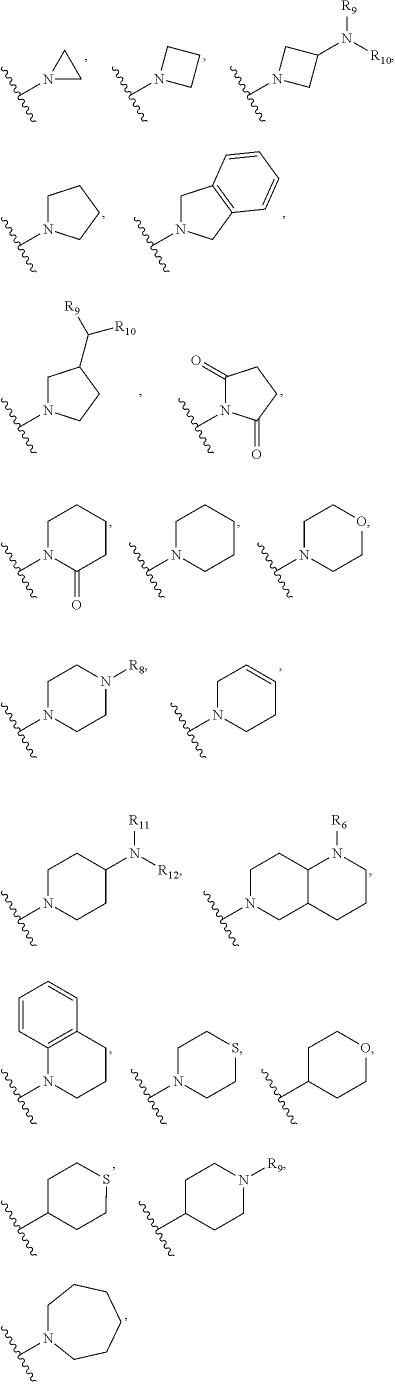 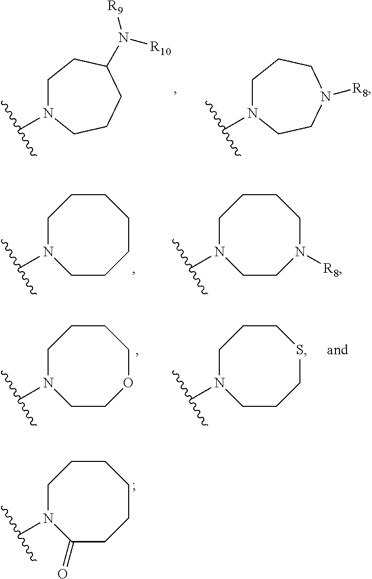
R³, R⁴, and R⁵ are independently selected from hydrogen, C₁-C₆ alkyl, C₁-C₆ alkenyl, C₁-C₆ alkynyl, C₁-C₆ alkoxy, C₃-C₆ cycloalkyl, phenyl, naphthyl, phenoxy, hydroxyl, amino, amide, oxo, —CN, and sulfonamide;
R⁶ and R⁷ are independently selected from hydrogen, C₁-C₆ alkyl, C₁-C₆ alkenyl, C₁-C₆ alkynyl, C₃-C₆ cycloalkyl, phenyl, naphthyl, halogen, hydroxyl, —CN, amino, and amido; and
R⁸ is selected from hydrogen, C₁-C₆ alkyl, C₁-C₆ alkenyl, C₁-C₆ alkynyl, acyl, and C₃-C₆ cycloalkyl; and
R⁹, R¹⁰, R¹¹, and R¹² are independently selected from hydrogen, C₁-C₆ alkyl, C₁-C₆ alkenyl, C₁-C₆ alkynyl, C₃-C₆ cycloalkyl, phenyl, naphthyl, heterocycle, hydroxyl, sulfonyl, and acyl; 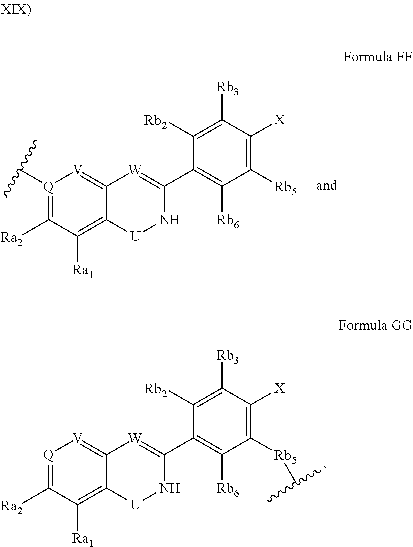
wherein:
Q is selected from N and CRa³;
V is selected from N and CRa⁴;
W is selected from N and CH;
U is selected from C═O, C═S, SO₂, S═O, and SR¹;
X is selected from OH, SH, NH₂, S(O)H, S(O)₂H, S(O)₂NH₂, S(O)NH₂, NHAc, and NHSO₂Me;
Ra¹, Ra³, and Ra³ are independently selected from hydrogen, C₁-C₆ alkyl, C₁-C₆ alkoxy, C₃-C₆ cycloalkyl, and halogen;
Ra² is selected from hydrogen, C₁-C₆ alkyl, C₁-C₆ alkoxy, C₃-C₆ cycloalkyl, amino, amide, and halogen;
Rb² and Rb⁶ are independently selected from hydrogen, methyl and fluorine;
Rb³ and Rb⁵ are independently selected from hydrogen, halogen, C₁-C₆ alkyl, C₃-C₆ cycloalkyl, and C₁-C₆ alkoxy; and
Rb² and Rb³ and/or Rb⁵ and Rb⁶ may be connected to form a cycloalkyl or a heterocycle, provided that at least one of Ra¹, Ra², Ra³, and Ra⁴ is not hydrogen; 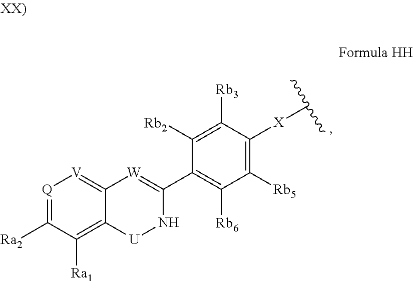
wherein:
Q is selected from N and CRa³;
V is selected from N and CRa⁴;
W is selected from N and CH;
U is selected from C═O, C═S, SO₂, S═O, and SR¹;
X is selected from OH, SH, NH₂, S(O)H, S(O)₂H, S(O)₂NH₂, S(O)NH₂, NHAc, and NHSO₂Me;
Ra¹, Ra³, and Ra³ are independently selected from hydrogen, C₁-C₆ alkyl, C₁-C₆ alkoxy, C₃-C₆ cycloalkyl, and halogen;
Ra² is selected from hydrogen, C₁-C₆ alkyl, C₁-C₆ alkoxy, C₃-C₆ cycloalkyl, amino, amide, and halogen;
Rb² and Rb⁶ are independently selected from hydrogen, methyl and fluorine;
Rb³ and Rb⁵ are independently selected from hydrogen, halogen, C₁-C₆ alkyl, C₃-C₆ cycloalkyl, and C₁-C₆ alkoxy; and
Rb² and Rb³ and/or Rb⁵ and Rb⁶ may be connected to form a cycloalkyl or a heterocycle, provided that at least one of Ra¹, Ra², Ra³, and Ra^{o} is not hydrogen; 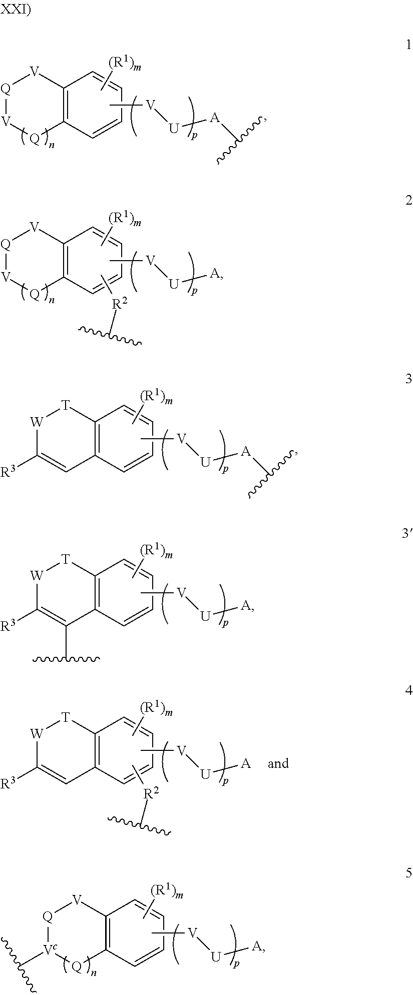
wherein:
V is independently selected, for each occurrence, from the group consisting of NH, S, N(C₁₋₆alkyl), O, or CR⁴R⁴;
Q is independently selected, for each occurrence, from the group consisting of C(O), C(S), C(N), SO₂, or CR⁴R⁴;
U is independently selected from the group consisting of a bond, C(O), C(S), C(N), SO₂, or CR⁴R⁴;
W and T are independently selected from the group consisting of NH, N(C₁₋₆alkyl), O, or Q;
V^{C} is selected from the group consisting of N, SH or CR⁴;
A is selected from the group consisting of aliphatic, cycloalkyl, heterocyclic, phenyl, naphthyl, heteroaryl or bicyclic moiety, wherein the cycloalkyl, heterocyclic, phenyl, naphthyl, heteroaryl, or bicyclic moiety is optionally substituted with one, two, three, four or more groups represented by R⁴;
R¹ is independently selected, for each occurrence, from the group consisting of hydroxyl, halo, C₁₋₆ alkyl, hydroxyC₁₋₆ alkyl, aminoC₁₋₆ alkyl, haloC₁₋₆ alkyl, C₁₋₆ alkoxy, haloC₁₋₆alkoxy, acylaminoC₁₋₆alkyl, nitro, cyano, CF₃, —OCF₃, —C(O)OC₁₋₆alkyl, —OS(O)₂C₁₋₄alkyl, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;
R² is selected from the group consisting of —O—, amino, C₁₋₆alkyl, —O—C₁₋₆alkyl-, hydroxylC₁₋₆ alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆alkyl-, —OS(O)₂C₁₋₄alkyl-, —OS(O)₂—, —S—C₁₋₆alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;
R³ is selected from the group consisting of hydrogen or C₁₋₆alkyl;
R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋₆alkyl, —NH—C₁₋₆alkyl, —N(C₁₋₆alkyl)C₁₋₆alkyl, nitro, cyano, CF₃, —OCF₃, —C(O)OC₁₋₆alkyl, —C(O)NHC₁₋₆alkyl, —C(O)NH₂ or —OS(O)₂C₁₋₄alkyl;
m is selected from the group consisting of 0, 1, 2, or 3;
n is selected from the group consisting of 0, 1, or 2; and
p is selected from the group consisting of 0 or 1; 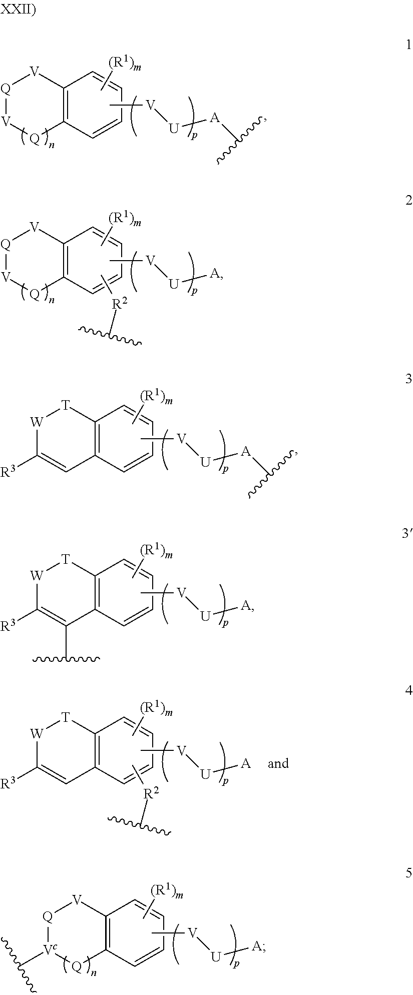
wherein:
V is independently selected, for each occurrence, from the group consisting of NH, S, N(C₁₋₆alkyl), O, or CR⁴R⁴;
Q is independently selected, for each occurrence, from the group consisting of C(O), C(S), C(N), SO₂, or CR⁴R⁴;
U is independently selected from the group consisting of a bond, C(O), C(S), C(N), SO₂, or CR⁴R⁴;
W and T are independently selected from the group consisting of NH, N(C₁₋₆alkyl), O, or Q;
V^{C} is selected from the group consisting of N, SH or CR⁴;
A is selected from the group consisting of aliphatic, cycloalkyl, heterocyclic, phenyl, naphthyl, heteroaryl or bicyclic moiety, wherein the cycloalkyl, heterocyclic, phenyl, naphthyl, heteroaryl, or bicyclic moiety is optionally substituted with one, two, three, four or more groups represented by R⁴;
R¹ is independently selected, for each occurrence, from the group consisting of hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆ alkyl, nitro, cyano, CF₃, —OCF₃, —C(O)OC₁₋₆ alkyl, —OS(O)₂C₁₋₄ alkyl, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆ alkyl, amino, or nitro;
R² is selected from the group consisting of —O—, amino, C₁₋₆alkyl, —O—C₁₋₆alkyl-, hydroxylC₁₋₆alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆ alkyl-, —OS(O)₂C₁₋₄ alkyl-, —OS(O)₂—, —S—C₁₋₆ alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;
R³ is selected from the group consisting of hydrogen or C₁₋₆alkyl;
R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋6alkyl, —NH—C₁₋₆ alkyl, —N(C₁₋₆ alkyl)C₁₋₆ alkyl, nitro, cyano, CF₃, —OCF₃, —C(O)OC₁₋₆ alkyl, —C(O)NHC₁₋₆alkyl, —C(O)NH₂ or —OS(O)₂C₁₋₄alkyl;
m is selected from the group consisting of 0, 1, 2, or 3;
n is selected from the group consisting of 0, 1, or 2; and
p is selected from the group consisting of 0 or 1; 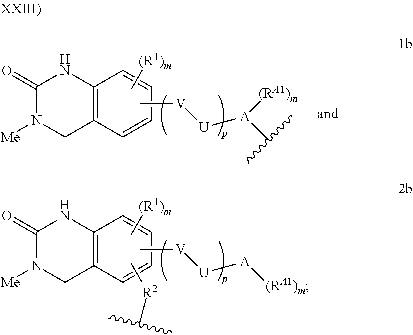
wherein:
V is selected from the group consisting of a NH, S, N(C₁₋₆alkyl), O, or CR⁴R⁴;
Q is selected from the group consisting of a bond, C(O), C(S), C(N), SO₂, or CR⁴R⁴;
A is a ring selected from the group consisting of: phenyl, a 5-6 membered cycloalkyl, a 5-6 membered heteroaryl having 1, 2 or 3 heteroatoms each selected from S, N or O, and a 4-7 membered heterocycle having 1, 2 or 3 heteroatoms each selected from N or O;
R^{A1} is R¹; or two R^{A1} substituents may be taken together with the atoms to which they are attached to form phenyl, a 5-6 membered heteroaryl having 1, 2 or 3 heteroatoms each selected from S, N or O, and a 4-7 membered heterocycle having 1, 2 or 3 heteroatoms each selected from N or O;
R¹ is independently selected, for each occurrence, from the group consisting of hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆alkyl, nitro, cyano, CF₃, —OCF₃, —C(O)OC₁₋₆alkyl, —OS(O)₂C₁₋₄alkyl, —S(C₁₋₄alkyl)C(O)R′, phenyl, naphthyl, phenyloxy, benzyloxy, or phenylmethoxy, wherein C₁₋₆alkyl, phenyl, and napththyl are optionally substituted by one two or three substituents selected from the group consisting of hydroxyl, halogen, oxo, C₁₋₆alkyl, amino, or nitro;
R² is selected from the group consisting of —O—, amino, C₁₋₆alkyl, —O—C₁₋₆alkyl-, hydroxylC₁₋₆alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆alkyl-, —OS(O)₂C₁₋₄alkyl-, —OS(O)₂—S(C₁₋₄alkyl)C(O)R″—, —S—C₁₋₆alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;
R³ is selected from the group consisting of hydrogen or C₁₋₆alkyl;
R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋₆alkyl, —NH—C₁₋₆alkyl, —N(C₁₋₆ alkyl)C₁₋₆ alkyl, nitro, cyano, CF₃, —OCF₃, —C(O)OC₁₋₆ alkyl, —C(O)NHC₁₋₆alkyl, —C(O)NH₂ or —OS(O)₂C₁₋₄alkyl;
R′ is independently selected, for each occurrence, from the group consisting of hydroxyl, amino, thio, phenyl, naphthyl, or C₁₋₆alkyl, wherein C₁₋₆alkyl, phenyl, and naphthyl are optionally substituted by one two or three substituents selected from the group consisting of hydroxyl, halogen, oxo, C₁₋₆ alkyl, amino, or nitro;
R″ is independently selected, for each occurrence, from the group consisting of —O—, amino, thio, phenyl, naphthyl, or C₁₋₆alkyl, wherein C₁₋₆alkyl, phenyl, and naphthyl are optionally substituted by one two or three substituents selected from the group consisting of hydroxyl, halogen, oxo, C₁₋₆ alkyl, amino, or nitro;
m is independently selected, for each occurrence, from the group consisting of 0, 1, 2, or 3;
n is selected from the group consisting of 0, 1, or 2; and
p is selected from the group consisting of 0 or 1; and 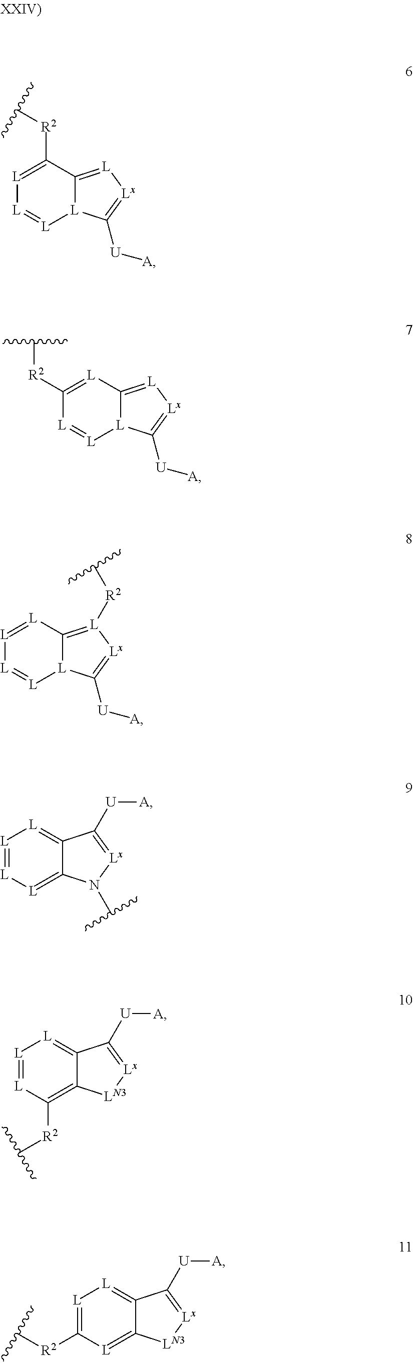 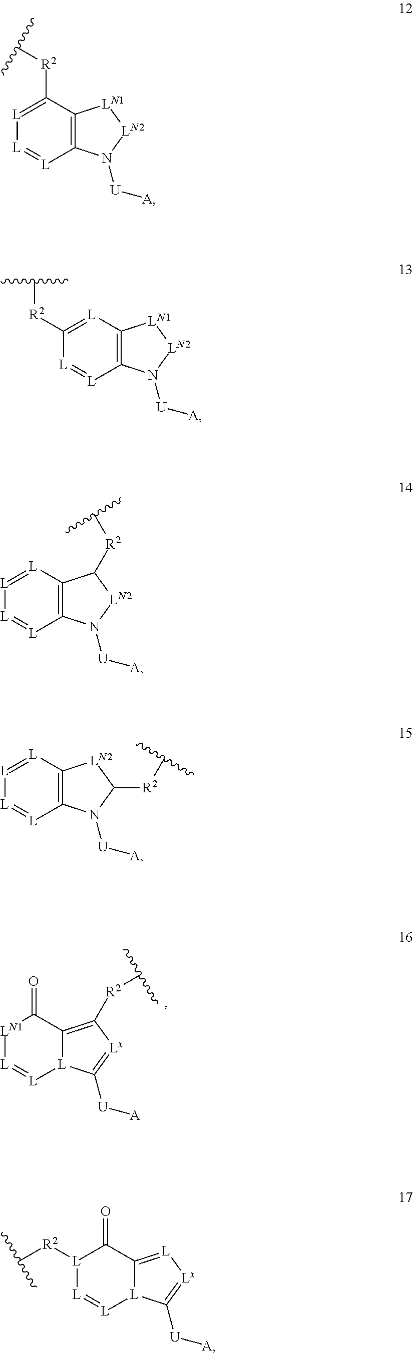 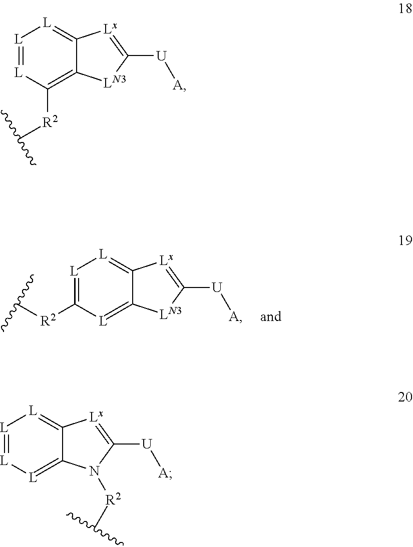
wherein:
L and L^{X} are independently selected, for each occurrence, from the group consisting of N, CH, and CR¹;
L^{N1} and L^{N2} are independently selected from the group consisting of CH₂, CHR¹, CR¹R¹, NH, and N(C₁₋₆alkyl); wherein C₁₋₆alkyl is optionally substituted by one two or three substituents selected from the group consisting of hydroxyl, halogen, oxo, C₁₋₆alkyl, amino, or nitro;
L^{N3} is selected from the group consisting of O, S, NH, and N(C₁₋₆alkyl); wherein C₁₋₆alkyl is optionally substituted by one two or three substituents selected from the group consisting of hydroxyl, halogen, oxo, C₁₋₆alkyl, amino, or nitro;
U is independently selected from the group consisting of a bond, C(O), C(S), C(N), SO₂, or CR⁴R⁴;
A is selected from the group consisting of aliphatic, cycloalkyl, heterocyclic, phenyl, naphthyl, heteroaryl, or bicyclic moiety, wherein the cycloalkyl, heterocyclic, phenyl, naphthyl, heteroaryl, or bicyclic moiety is optionally substituted with one, two, three, four or more groups represented by R⁴;
R¹ is independently selected, for each occurrence, from the group consisting of hydroxyl, halo, C₁₋₆alkyl, hydroxyC₁₋₆alkyl, aminoC₁₋₆alkyl, haloC₁₋₆alkyl, C₁₋₆alkoxy, haloC₁₋₆alkoxy, acylaminoC₁₋₆ alkyl, nitro, cyano, CF₃, —OCF₃, —C(O)OC₁₋₆ alkyl, —OS(O)₂C₁₋₄ alkyl, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆ alkyl, amino, or nitro;
R² is selected from the group consisting of —O—, amino, C₁₋₆alkyl, —O—C₁₋₆alkyl-, hydroxylC₁₋₆alkyl, aminoC₁₋₆ alkyl, haloC₁₋₆ alkyl, haloC₁₋₆ alkoxy, acylaminoC₁₋₆ alkyl, —C(O)—, —C(O)O—, —C(O)NC₁₋₆ alkyl-, —OS(O)₂C₁₋₄ alkyl-, —OS(O)₂—, —S—C₁₋₆ alkyl-, phenyl, naphthyl, phenyloxy, benzyloxy, or phenylmethoxy, wherein C₁₋₆alkyl, phenyl, and naphthyl are optionally substituted by one two or three substituents selected from the group consisting of hydroxyl, halogen, oxo, C₁₋₆alkyl, amino, or nitro;
R³ is selected from the group consisting of hydrogen or C₁₋₆alkyl; and
R⁴ is independently selected, for each occurrence, from the group consisting of hydrogen, hydroxyl, oxo, imino, amino, halo, C₁₋₆alkyl, cycloalkyl, phenyl, naphthyl, heterocyclyl, —O—C₁₋₆alkyl, —NH—C₁₋₆alkyl, —N(C₁₋₆ alkyl)C₁₋₆ alkyl, nitro, cyano, CF₃, —OCF₃, —C(O)OC₁₋₆ alkyl, —C(O)NHC₁₋₆alkyl, —C(O)NH₂ or —OS(O)₂C₁₋₄alkyl.

7. The first monomer of claim 1, wherein X¹ and X² are each independently selected from the group consisting of:

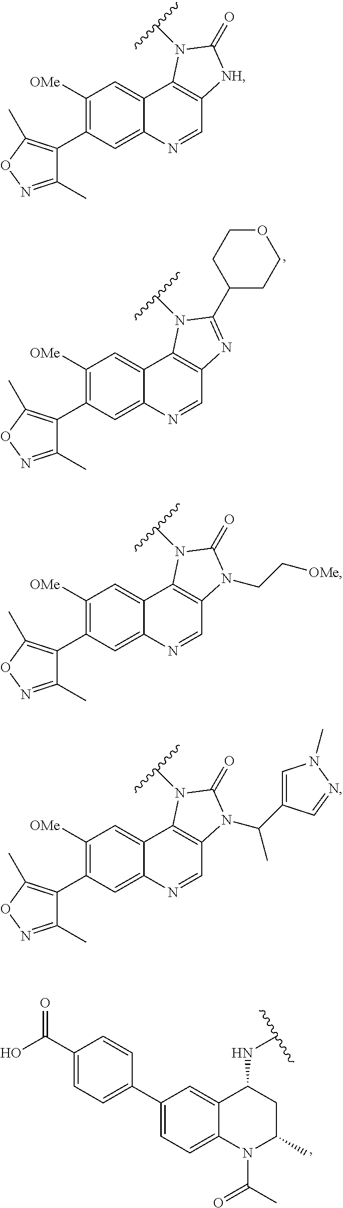 [SMILES: *n1c(=O)[nH]c2cnc3cc(-c4c(C)noc4C)c(OC)cc3c21 ; *n1c(C2CCOCC2)nc2cnc3cc(-c4c(C)noc4C)c(OC)cc3c21 ; *n1c(=O)n(CCOC)c2cnc3cc(-c4c(C)noc4C)c(OC)cc3c21 ; *n1c(=O)n(C(C)c2cnn(C)c2)c2cnc3cc(-c4c(C)noc4C)c(OC)cc3c21 ; *N[C@@H]1C[C@H](C)N(C(C)=O)c2ccc(-c3ccc(C(=O)O)cc3)cc21]

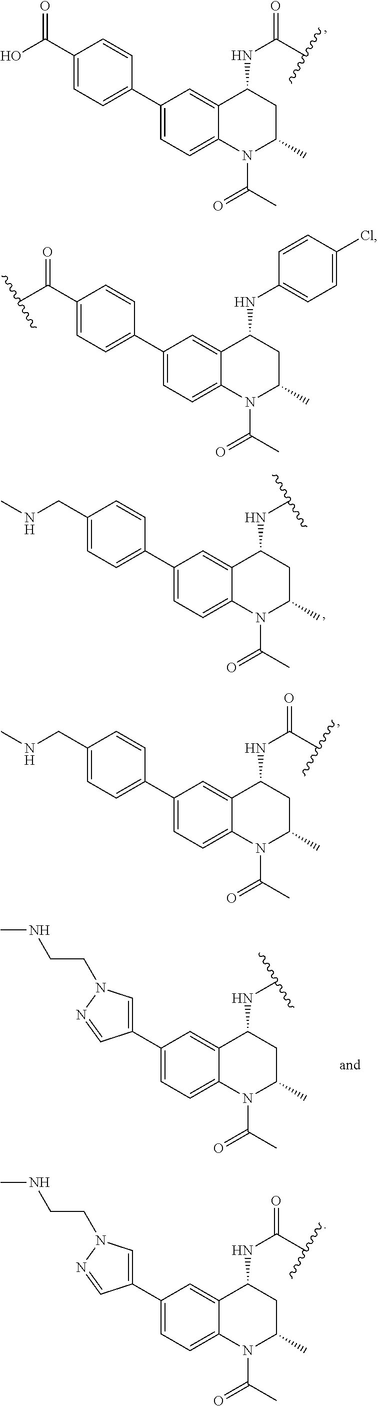

8. The first monomer of claim 1, wherein X¹ and X² are each selected independently from the group consisting of:

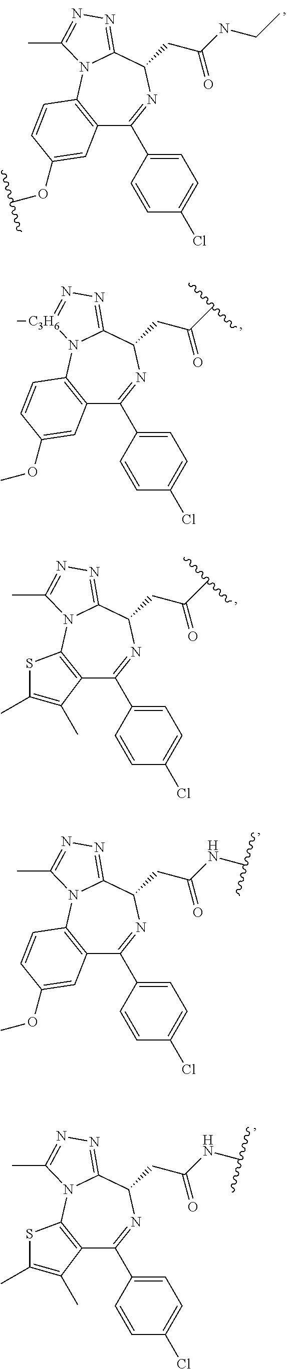

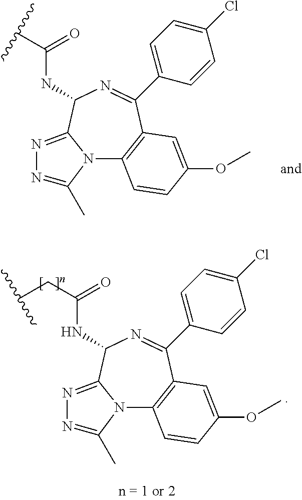

9. The first monomer of claim 1, wherein X¹ and X² are each selected independently from the group consisting of:

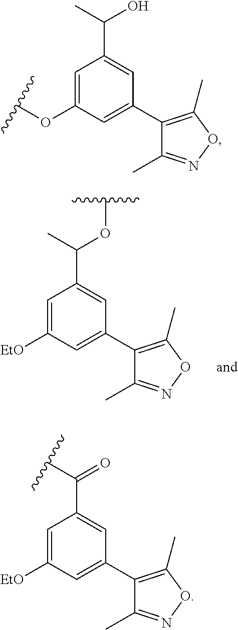

10. The first monomer of claim 1, wherein X¹ and X² are the same.

11. The first monomer of claim 1, wherein X¹ and X² are different.

12. The first monomer of claim 1, wherein the first monomer forms a biologically useful dimer with a second monomer in vivo.

13. The first monomer of claim 1, wherein Z₁ is selected from the group consisting of: 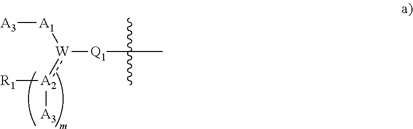
wherein
A₁ is (a) absent; or (b) selected from the group consisting of acyl, substituted or unsubstituted aliphatic, or substituted or unsubstituted heteroaliphatic;
A₂, independently for each occurrence, is (a) absent; or (b) selected from the group consisting of —N—, acyl, substituted or unsubstituted aliphatic, or substituted or unsubstituted heteroaliphatic, provided that at least one of A₁ and A₂ is present; or
A₁ and A₂, together with the atoms to which they are attached, form a substituted or unsubstituted 4-8 membered cycloalkyl or heterocyclic ring;
A₃ is selected from the group consisting of —NHR′, —SH, or —OH;
W is CR′ or N;
R′ is selected from the group consisting of hydrogen, halogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH;
m is 1-6; [symbol]
represents a single or double bond; and
R₁ is (a) absent; or (b) selected from the group consisting of hydrogen, halogen, substituted or unsubstituted aliphatic, or substituted or unsubstituted heteroaliphatic, substituted or unsubstituted phenyl or naphthyl, substituted or unsubstituted heteroaryl, —NH₂, —NO₂, —SH, or —OH;
Q₁ is (a) absent; or (b) selected from the group consisting of substituted or unsubstituted aliphatic or substituted or unsubstituted heteroaliphatic; or
R₁ and Q₁ together with the atoms to which they are attached form a substituted or unsubstituted 4-8 membered cycloalkyl or heterocyclic ring; 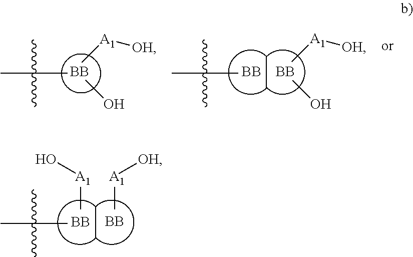
wherein
BB, independently for each occurrence, is a 4-8 membered cycloalkyl, heterocyclic, phenyl, naphthyl, or heteroaryl moiety, wherein the cycloalkyl, heterocyclic, phenyl, naphthyl, or heteroaryl moiety is optionally substituted with one or more groups represented by R₂, wherein the two substituents comprising —OH have a 1,2 or 1,3 configuration;
each R₂ is independently selected from hydrogen, halogen, oxo, sulfonate, —NO₂, —CN, —OH, —NH₂, —SH, —COOH, —CONHR′, substituted or unsubstituted aliphatic, substituted or unsubstituted heteroaliphatic, or two R₂ together with the atoms to which they are attached form a fused substituted or unsubstituted 4-6 membered cycloalkyl or heterocyclic bicyclic ring system;
A₁, independently for each occurrence, is (a) absent; or (b) selected from the group consisting of acyl, substituted or unsubstituted aliphatic, or substituted or unsubstituted heteroaliphatic;
R′ is selected from the group consisting of hydrogen, halogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH; 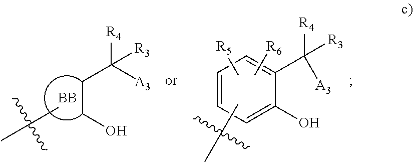
wherein
BB is a substituted or unsubstituted 5- or 6-membered cycloalkyl, heterocyclic, phenyl or naphthyl, or heteroaryl moiety;
A₃, independently for each occurrence, is selected from the group consisting of —NHR′ or —OH;
R₃ and R₄ are independently selected from the group consisting of H, C₁₋₄alkyl, phenyl, or R₃ and R₄ taken together from a 3-6 membered ring;
R₅ and R₆ are independently selected from the group consisting of H, C₁₋₄alkyl optionally substituted by hydroxyl, amino, halogen, or thio; C₁₋₄alkoxy; halogen; —OH; —CN; —COOH; —CONHR′; or R₅ and R₆ taken together form phenyl or a 4-6 membered heterocycle; and
R′ is selected from the group consisting of hydrogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH; 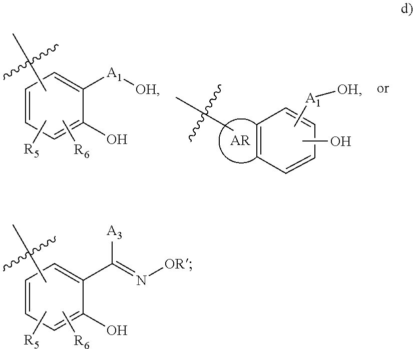
wherein
A₁ is (a) absent; or (b) selected from the group consisting of acyl, substituted or unsubstituted aliphatic, or substituted or unsubstituted heteroaliphatic;
A₃, independently for each occurrence, is selected from the group consisting of —NHR′ or —OH;
AR is a fused phenyl or 4-7 membered aromatic or partially aromatic heterocyclic ring, wherein AR is optionally substituted by oxo, C₁₋₄alkyl optionally substituted by hydroxyl, amino, halo, or thio; C₁₋₄alkoxy; —S—C₁₋₄alkyl; halogen; —OH; —CN; —COOH; —CONHR′; wherein the two substituents comprising —OH are ortho to each other;
R₅ and R₆ are independently selected from the group consisting of H, C₁₋₄alkyl optionally substituted by hydroxyl, amino, halo, or thio; C₁₋₄alkoxy; halogen; —OH; —CN; —COOH; CONHR′; and
R′ is selected from the group consisting of hydrogen, halogen, substituted or unsubstituted aliphatic, substituted or unsubstituted heteroaliphatic, substituted or unsubstituted phenyl or naphthyl, substituted or unsubstituted heteroaryl, —NH₂, —NO₂, —SH, or —OH; 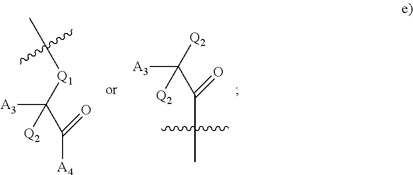
wherein
Q₁ is selected from the group consisting of C₁₋₄alkyl, alkylene, or a bond; C₁₋6cycloalkyl; a 5-6 membered heterocyclic ring; or phenyl;
Q₂, independently for each occurrence, is selected from the group consisting of H, C₁₋₄alkyl, alkylene, or a bond; C₁₋₆cycloalkyl; a 5-6 membered heterocyclic ring; substituted or unsubstituted aliphatic; substituted or unsubstituted heteroaliphatic; substituted or unsubstituted phenyl or naphthyl; or substituted or unsubstituted heteroaryl;
A₃, independently for each occurrence, is selected from the group consisting of —NH₂ or —OH;
A₄, independently for each occurrence, is selected from the group consisting of —NH—NH₂; —NHOH, —NH—OR″, or —OH;
R″ is selected from the group consisting of H or C₁₋₄alkyl; and 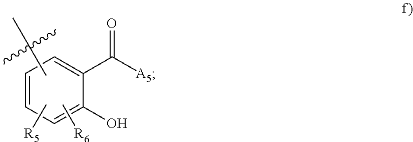
wherein
A₅ is selected from the group consisting of —OH, —NH₂, —SH, —NHR′″;
R′″ is selected from —NH₂; —OH; phenoxy; and C₁₋₄alkoxy;
R₅ and R₆ are independently selected from the group consisting of H, C₁₋₄alkyl optionally substituted by hydroxyl, amino, halo, or thio; C₁₋₄alkoxy; halogen; —OH; —CN; —COOH; —CONHR′; or R₅ and R₆ taken together may form a 5-6 membered ring;
R′ is selected from the group consisting of hydrogen, substituted or unsubstituted aliphatic, substituted or unsubstituted heteroaliphatic, substituted or unsubstituted phenyl or naphthyl, substituted or unsubstituted heteroaryl, —NH₂, —SH, or —OH; and
the second monomer has a boronic acid or oxaborole moiety capable of binding with the Z₁ moiety of Formula I to form the multimer.

14. The first monomer of claim 1, wherein the aqueous fluid has a physiologically acceptable pH.

15. The first monomer of claim 13, wherein Z₂ of the second monomer is selected from the group consisting of: 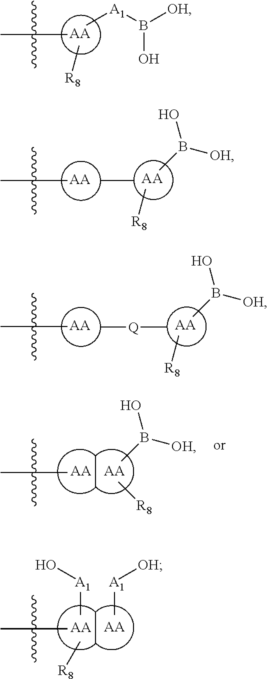
wherein
R₈ is selected from the group consisting of H, halogen, oxo, C₁₋₄alkyl optionally substituted by hydroxyl, amino, halo or thio; C₂₋₄alkenyl, C₁₋₄alkoxy; —S—C₁₋₄alkyl; —CN; —COOH; or —CONHR′;
A₁ is (a) absent; or (b) selected from the group consisting of acyl, substituted or unsubstituted aliphatic, or substituted or unsubstituted heteroaliphatic;
Q is selected from the group consisting of substituted or unsubstituted aliphatic, or substituted or unsubstituted heteroaliphatic;
AA, independently for each occurrence, is phenyl, naphthyl, or a 5-7 membered heterocyclic or heteroaryl ring having one, two, or three heteroatoms, wherein AA is optionally substituted by one, two, or three substituents selected from the group consisting of halogen, C₁₋₄alkyl optionally substituted by hydroxyl, amino, halogen, or thio; C₂₋₄alkenyl, C₁₋₄alkoxy; —S—C₁₋₄alkyl; —CN; —COOH; —CONHR′; or two substituents together with the atoms to which they are attached form a fused 4-6 membered cycloalkyl or heterocyclic bicyclic ring system; and
R′ is H or C₁₋₄alkyl.

16. A therapeutic multimer compound formed from the multimerization in an aqueous media of a first monomer represented by:
X¹—Y¹—Z¹ (Formula I)
and a second monomer represented by
X²—Y²—Z² (Formula II),
wherein
X¹ is a first ligand moiety capable of modulating a first bromodomain;
Y¹ is absent or is a connector moiety covalently bound to X¹ and Z¹;
Z¹ is a first linker capable of binding to Z² to form the multimer;
X² is a second ligand moiety capable of modulating a second protein domain;
Y² is absent or is a connector moiety covalently bound to X² and Z²; and
Z² is a boronic acid or oxaborale moiety capable of binding with the Z¹ moiety of Formula I to form the multimer; and
pharmaceutically acceptable salts, stereoisomers, metabolites and hydrates thereof.

17. The therapeutic multimer compound of claim 16, wherein X¹ and X² are each independently selected from the group consisting of:

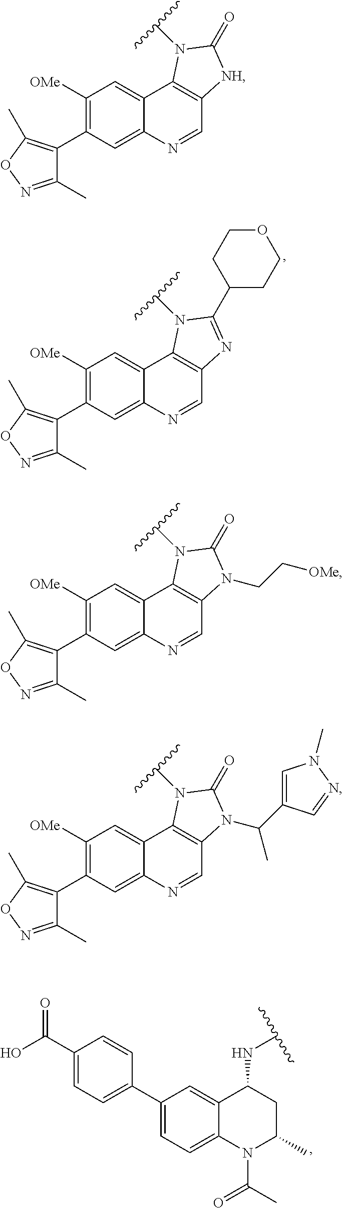 [SMILES: *n1c(=O)[nH]c2cnc3cc(-c4c(C)noc4C)c(OC)cc3c21 ; *n1c(C2CCOCC2)nc2cnc3cc(-c4c(C)noc4C)c(OC)cc3c21 ; *n1c(=O)n(CCOC)c2cnc3cc(-c4c(C)noc4C)c(OC)cc3c21 ; *n1c(=O)n(C(C)c2cnn(C)c2)c2cnc3cc(-c4c(C)noc4C)c(OC)cc3c21 ; *N[C@@H]1C[C@H](C)N(C(C)=O)c2ccc(-c3ccc(C(=O)O)cc3)cc21]

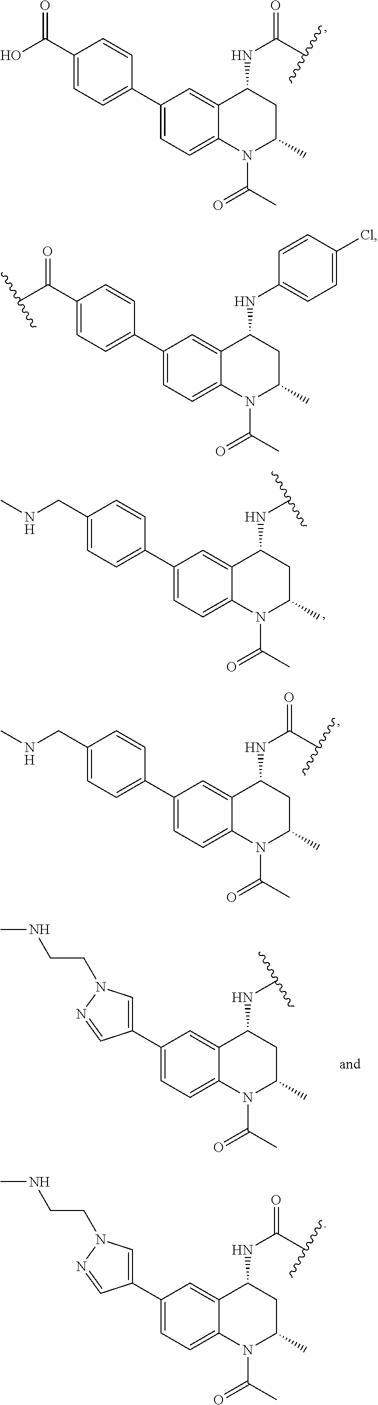

18. The therapeutic multimer compound of claim 16, wherein X¹ and X² are each independently selected from the group consisting of:

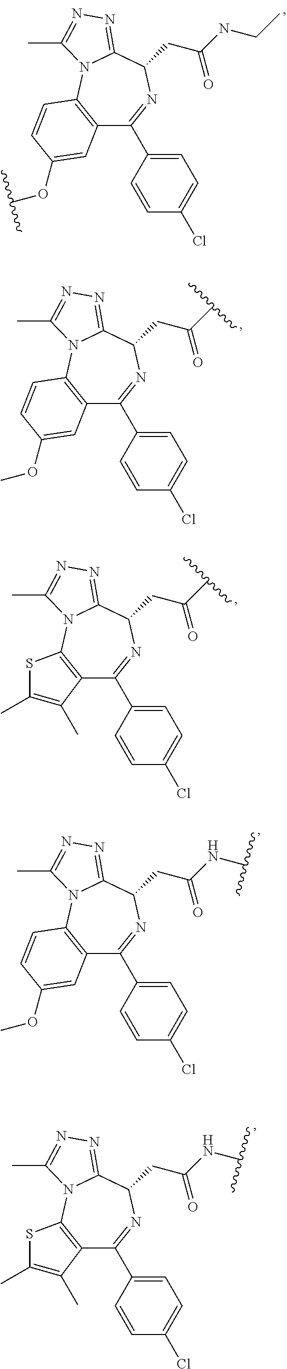

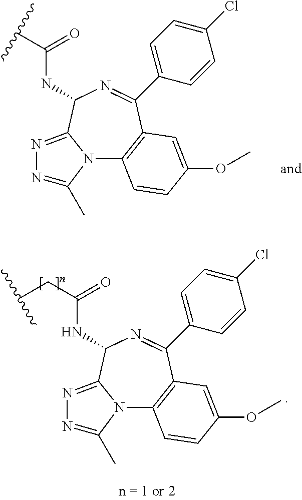

19. The therapeutic multimer compound of claim 16, wherein X¹ and X² are each independently selected from the group consisting of:

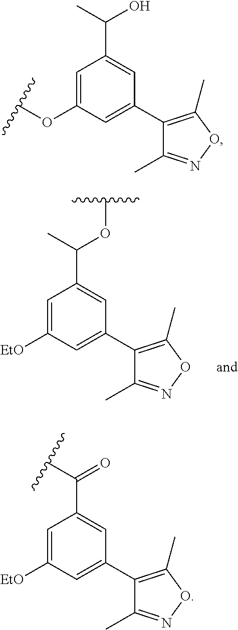

20.-28. (canceled)

29. A method of treating a disease associated with a protein having tandem bromodomains in a patient in need thereof comprising:
administering to said patient a first monomer represented by:
X¹—Y¹—Z¹ (Formula I)
and pharmaceutically acceptable salts, stereoisomers, metabolites and hydrates thereof, wherein
X¹ is a first ligand moiety capable of modulating a first bromodomain; and
administering to said patient a second monomer represented by:
X²—Y²—Z² (Formula II),
wherein
X² is a second ligand moiety capable of modulating a second bromodomain, wherein upon administration, said first monomer and said second monomer forms a multimer in vivo that binds to the first and the second bromodomain.

30. (canceled)
